(19) United States
(12) Patent Application Publication
(10) Pub. No.: US 2013/0190289 A1
(43) Pub. Date: Jul. 25, 2013

(54) COMPOUNDS AND METHODS FOR THE TREATMENT OR PREVENTION OF FLAVIVIRUS INFECTIONS

(71) Applicant: Vertex Pharmaceuticals Incorporated, Cambridge, MA (US)

(72) Inventors: Jeremy Green, Waltham, MA (US); Dean M. Wilson, Bedford, MA (US); Laval Chan Chun Kong, Kirkland (CA); Sanjoy Kumar Das, Pierrefonds (CA); Carl Poisson, Montreal (CA); John J. Court, Littleton, MA (US); Qing Tang, Acton, MA (US); Pan Li, Lexington, MA (US); Philip N. Collier, Arlington, MA (US); Nathan Waal, Cambridge, MA (US); David J. Lauffer, Stow, MA (US); Warren Dorsch, Providence, RI (US)

(73) Assignee: Vertex Pharmaceuticals Incorporated, Cambridge, MA (US)

(21) Appl. No.: 13/723,668

(22) Filed: Dec. 21, 2012

Related U.S. Application Data
(63) Continuation of application No. PCT/US2011/042110, filed on Jun. 28, 2011.
(60) Provisional application No. 61/467,653, filed on Mar. 25, 2011.
(60) Provisional application No. 61/359,169, filed on Jun. 28, 2010.

(51) Int. Cl. C07D 413/12; C07D 409/12; C07D 333/38

(52) U.S. Cl. CPC C07D 413/12; C07D 333/38; C07D 409/12
USPC 514/210.18; 549/480; 514/447; 548/527; 514/422; 544/146; 514/231.5; 548/952; 540/524; 514/212.08; 540/488; 514/211.03; 435/184

(57) ABSTRACT

A compound is represented by Structural Formula (I):

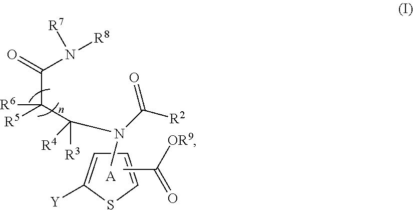

or a pharmaceutically acceptable salt thereof, wherein the variables of Structural Formula (I) are as described in the specification and the claims. A pharmaceutical composition comprises a compound represented by Structural Formula (I) or a pharmaceutically acceptable salt thereof, and a pharmaceutically acceptable carrier of excipient. A method of treating a HCV infection in a subject comprises administering to the subject a therapeutically effective amount of a compound represented by Structural Formula (I) or a pharmaceutically acceptable salt thereof. A method of inhibiting or reducing the activity of HCV polymerase in a subject or in a biological in vitro sample comprises administering to the subject or to the sample a therapeutically effective amount of a compound represented by Structural Formula (I) or a pharmaceutically acceptable salt thereof.

### RELATED APPLICATIONS

This application is a continuation of PCT Application Number PCT/US2011/042110, filed Jun. 28, 2011, which claims priority to U.S. Provisional Applications, U.S. Ser. No. 61/359,169 filed on Jun. 28, 2010 and to U.S. Ser. No. 61/467,653 filed on Mar. 25, 2011. The entire teachings of these applications are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co- and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

There is therefore a great need for the development of anti-viral agents for use in treating or preventing Flavivirus infections.

### SUMMARY OF THE INVENTION

The present invention generally relates to compounds useful for treating or preventing Flavivirus infections, such as HCV infections.

In one embodiment, the invention is directed to a compound represented by Structural Formula (I) or (II):

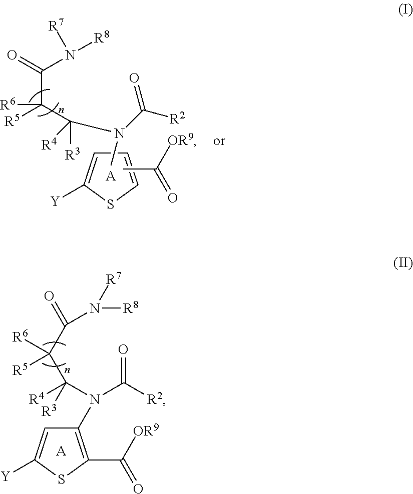

or a pharmaceutically acceptable salt thereof, wherein the variables are as described below:

Ring A is optionally further substituted with one or more substituents selected from the group consisting of -D (deuterium), halogen, —CN, C₁₋₆ alkyl, and C₁₋₆ haloalkyl.

Y is C₃₋₈ carbocycle, 5-8 membered heterocycle, —(C₂ aliphatic group)-R¹, C₆₋₁₀ aryl, or 5-10 membered heteroaryl, wherein each of said carbocycle, heterocycle, aryl and heteroaryl is optionally and independently substituted with one or more instances of J^{Y} independently selected from the group consisting of halogen, —CN, nitro, azido, R^{a}, —SO₂R^{a}, —OR^{a}, —COR^{a}, —NRR^{a}, —C(O)OR^{a}, —OC(O)R^{a}, —NRC(O)R^{a}, —C(O)NRR^{a}, —NRC(O)NRR^{a}, —NRC(O)OR^{a}, —OCONRR^{a}, —SO₂NRR^{a}, —NRSO₂R^{a}, —NRSO₂NRR^{a}, and —NRC(═NR)NRR^{a}, and wherein said C₂ aliphatic group is optionally substituted with one or more substitutents selected from the group consisting of halogen, —CN, C₁₋₂ alkyl, C₁₋₂ haloalkyl, hydroxy, and methoxy.

R¹ is —H or a C₁₋₆ alkyl, C₃₋₁₀ carbocyclic, 4-10 membered heterocyclic, C₆₋₁₀ aryl, or 5-10 membered heteroaryl group, wherein said alkyl group is optionally substituted with one or more instances of J^{1A}, and wherein each of said carbocyclic and heterocyclic groups is optionally and independently substituted with one or more instances of J^{1B}, and wherein each of said aryl and heteroaryl groups is optionally and independently substituted with one or more instances of J^{1C}.

R² is a C₁₋₆ aliphatic, C₃₋₁₀ carbocyclic, 4-10 membered heterocyclic, C₆₋₁₀ aryl, or 5-10 membered heteroaryl group, wherein said aliphatic group is optionally substituted with one or more instances of J^{2A}, each of said carbocyclic and heterocyclic groups is independently and optionally substituted with one or more instances of J^{2B}, and each of said aryl and heteroaryl groups is independently and optionally substituted with one or more instances of J^{2C}.

Each of R³, R⁴, R⁵ and R⁶ independently is —H, -D, or a C₁₋₆ aliphatic group optionally substituted with one or more instances of J^{D}.

Each of R⁷ and R⁸ independently is —H or a C₁₋₆ aliphatic, C₃₋₄₀ carbocyclic, 4-10 membered heterocyclic, C₆₋₁₀ aryl, or 5-10 membered heteroaryl group, wherein said aliphatic group is optionally substituted with one or more instances of J^{7A}, and wherein each of said carbocyclic and heterocyclic groups is independently and optionally substituted with one or more instances of J^{7B}, and wherein each of said aryl and heteroaryl groups is independently and optionally substituted with one or more instances of J^{7C}; or

R⁷ and R⁸, together with the nitrogen atom to which they are attached, form a 4-10 membered heterocyclic ring (e.g., monocyclic, bicyclic, or tricyclic ring, fused ring, bridged ring, or spiro ring), such as 4-8 membered heterocyclic ring, that is optionally substituted with one or more instances of J^{E}.

Optionally, when Y is —(C₂ aliphatic group)-R¹, R³ and R⁷, together with the atoms to which they are attached, form a 4-10 membered heterocyclic ring optionally substituted with one or more instances of J^{E}.

R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}.

Each of J^{1A}, J^{2A}, J^{7A} and J^{9A} independently is oxo, Q; or two J^{1A}, two J^{2A}, two J^{7A}, and two J^{9A}, respectively, together with the atom(s) to which they are attached, optionally and independently form a 3-8-membered non-aromatic ring that is optionally substituted with one or more instances of J^{E}.

Each of J^{1B}, J^{2B}, J^{7B}, and independently is oxo or Q, or a C₁₋₆ aliphatic group optionally substituted with one or more instances of Q; or two J^{1B}, two J^{2B}, two J^{3B}, two J^{7B}, and two J^{9B}, respectively, together with the atom(s) to which they are attached, optionally and independently form a 3-8-membered non-aromatic ring that is optionally substituted with one or more instances of J^{E}.

Each of J^{1C}, J^{2C}, J^{7C} and J^{9C} independently is Q or a C₁₋₆ aliphatic group optionally substituted with one or more instances of Q; or two J^{1C}, two J^{2C}, two J^{7C}, and two J^{9C} respectively, together with the atom(s) to which they are attached, optionally and independently form a 3-8-membered non-aromatic ring that is optionally substituted with one or more instances of J^{E}.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}.

Each R^{a} independently is: i) —H; ii) a C₁₋₆ aliphatic group optionally substituted with one or more substituents independently selected from the group consisting of halogen, oxo, —CN, —OR, —NRR′, —OCOR, —COR″, —CO₂R, —CONRR′, —NRC(O)R, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or

R^{a}, together with R and the nitrogen atom to which it is attached, optionally forms a 4-8 membered heterocycle optionally substituted with one or more instances of J^{E}.

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or R′, together with R and the nitrogen atom to which it is attached, optionally forms a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.

Each J^{E} is independently selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1.

Exemplary heterocyclic rings formed with R³ and R⁷, together with the nitrogen atom to which they are attached, include:

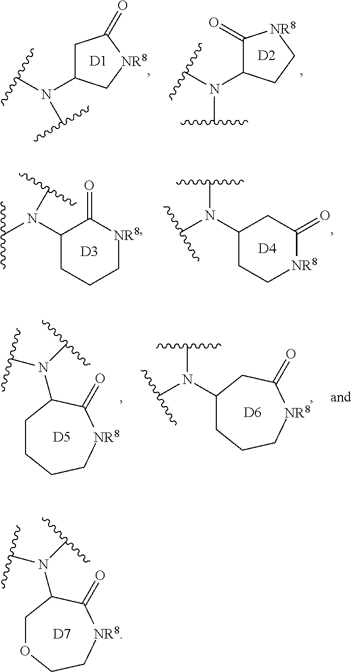

In some embodiments, R⁸ in rings D1-D7 are each independently —H or optionally substituted C₁₋₆alkyl. In other embodiments, R⁸ in rings D1-D7 are each independently —H or C₁₋₆ alkyl.

In another embodiment, the invention is directed to a compound represented by Structural Formula (III) or (IV):

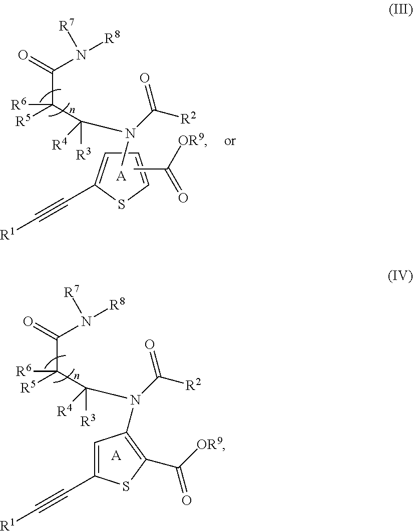

or a pharmaceutically acceptable salt thereof, wherein the values of the variables of Structural Formulae (III) and (IV) are independently as described below:

Ring A is optionally further substituted with one or more substituents selected from the group consisting of -D (deuterium), halogen, —CN, C₁₋₆alkyl, and C₁₋₆ haloalkyl.

R¹ is —H or a C₁₋₆alkyl, C₃₋₁₀ carbocyclic, 4-10 membered heterocyclic, C₆₋₁₀ aryl, or 5-10 membered heteroaryl group, wherein said aliphatic group is optionally substituted with one or more instances of J^{1A}, and wherein each of said carbocyclic and heterocyclic groups is optionally and independently substituted with one or more instances of J^{1B}, and wherein each of said aryl and heteroaryl groups is optionally and independently substituted with one or more instances of J^{1C}.

R² is a C₁₋₆ aliphatic, C₃₋₁₀ carbocyclic, 4-10 membered heterocyclic, C₆₋₁₀ aryl, or 5-10 membered heteroaryl group, wherein said aliphatic group is optionally substituted with one or more instances of J^{2A}, each of said carbocyclic and heterocyclic groups is independently and optionally substituted with one or more instances of J^{2B}, and each of said aryl and heteroaryl groups is independently and optionally substituted with one or more instances of J^{2C}.

Each of R³, R⁴, R⁵ and R⁶ independently is —H, -D (deuterium), or a C₁₋₆ aliphatic group optionally substituted with one or more instances of J^{D}.

Optionally, R³ and R⁷, together with the atoms to which they are attached, form a 4-10 membered heterocyclic ring (such as 4-8 membered heterocyclic ring) optionally substituted with one or more instances of J^{E}.

Each of R⁷ and R⁸ independently is —H or a C₁₋₆ aliphatic, C₃₋₁₀ carbocyclic, 4-10 membered heterocyclic, C₆₋₁₀ aryl, or 5-10 membered heteroaryl group, wherein said aliphatic group is optionally substituted with one or more instances of J^{7A}, and wherein each of said carbocyclic and heterocyclic groups is independently and optionally substituted with one or more instances of J^{7B}, and wherein each of said aryl and heteroaryl groups is independently and optionally substituted with one or more instances of J^{7C}; or

Optionally, R⁷ and R⁸, together with the nitrogen atom to which they are attached, form a 4-10 membered heterocyclic ring (e.g., monocyclic, bicyclic, or tricyclic ring, fused ring, bridged ring, or spiro ring), such as 4-8 membered heterocyclic ring, that is optionally substituted with one or more instances of J^{E}.

R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}.

Each of J^{1A}, J^{2A}, J^{7A}, and J^{9A} independently is oxo or Q; or two J^{1A}, two J^{2A}, two J^{7A}, and two J^{9A}, respectively, together with the atom(s) to which they are attached, optionally and independently form a 3-8-membered non-aromatic (carbocyclic or heterocyclic) ring that is optionally substituted with one or more instances of J^{E}.

Each of J¹, J^{2B}, J^{7B}, and J^{9B} and independently is oxo or Q, or a C₁₋₆ aliphatic group optionally substituted with one or more instances of Q; or two J^{1B}, two J^{2B}, two J^{3B}, two J^{7B}, and two J^{9B}, respectively, together with the atom(s) to which they are attached, optionally and independently form a 3-8-membered non-aromatic (carbocyclic or heterocyclic) ring that is optionally substituted with one or more instances of J^{E}.

Each of J^{1C}, J^{2C}, J^{7C} and J^{9C} independently is Q or a C₁₋₆ aliphatic group optionally substituted with one or more instances of Q; or two J^{1C}, two J^{2C}, two J^{7C}, and two J^{9C} respectively, together with the atom(s) to which they are attached, optionally and independently form a 3-8-membered non-aromatic (carbocyclic or heterocyclic) ring that is optionally substituted with one or more instances of J^{E}.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}.

Each R^{a} independently is: i) —H; ii) a C₁₋₆ aliphatic group optionally substituted with one or more substituents independently selected from the group consisting of halogen, oxo, —CN, —OR, —NRR′, —OCOR, —COR″, —CO₂R, —CONRR′, —NRC(O)R, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or

R^{a}, together with R and the nitrogen atom to which it is attached, optionally forms a 4-8 membered heterocycle optionally substituted with one or more instances of J^{E}.

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or R′, together with R and the nitrogen atom to which it is attached, optionally forms a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.

Each J^{E} is independently selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1.

In yet another embodiment, the invention is directed to a pharmaceutical composition comprising a compound of the invention described herein (e.g., a compound selected from the compounds described in the claims and FIGS. 1-3, such as a compound represented by any one of Structural Formulae (I)-(XIII), (XX), and (XXI), or a pharmaceutically acceptable salt thereof) and a pharmaceutically acceptable carrier or excipient.

In yet another embodiment, the invention provides methods of treating a HCV infection in a subject, comprising administering to the subject a therapeutically effective amount of a compound of the invention described herein a compound of the invention described herein (e.g., a compound selected from the compounds described in the claims and FIGS. 1-3, such as a compound represented by any one of Structural Formulae (I)-(XIII), (XX), and (XXI), or a pharmaceutically acceptable salt thereof).

In yet another embodiment, the invention is directed to a method of inhibiting or reducing the activity of HCV polymerase in a subject, comprising administering to the subject a therapeutically effective amount of a compound of the invention described herein (e.g., a compound selected from the compounds described in the claims and FIGS. 1-3, such as a compound represented by any one of Structural Formulae (I)-(XIII), (XX), and (XXI), or a pharmaceutically acceptable salt thereof).

In yet another embodiment, the invention is directed to a method of inhibiting or reducing the activity of HCV polymerase in a biological in vitro sample, comprising administering to the sample an effective amount of a compound of the invention described herein (e.g., a compound selected from the compounds described in the claims and FIGS. 1-3, such as a compound represented by any one of Structural Formulae (I)-(XIII), (XX), and (XXI), or a pharmaceutically acceptable salt thereof).

The present invention also provides use of the compounds of the invention described herein (e.g., the compounds described in the claims and FIGS. 1-3, such as the compounds represented by Structural Formulae (I)-(XIII), (XX), and (XXI), or pharmaceutically acceptable salts thereof), for the manufacture of the medicament for treating a HCV infection in a subject, or for inhibiting or reducing the activity of HCV polymerase in a subject.

Also provided herein is use of the compounds of the invention described herein (e.g., the compounds described in the claims and FIGS. 1-3, such as the compounds represented by Structural Formulae (I)-(XIII)), (XX), and (XXI), or pharmaceutically acceptable salts thereof), for treating a HCV infection in a subject, or for inhibiting or reducing the activity of HCV polymerase in a subject. Without being bound to a particular theory, the compounds of the invention are generally NS5B inhibitors.

### DESCRIPTION OF THE DRAWINGS

FIGS. 1-3 show tables depicting certain compounds of the invention.

### DETAILED DESCRIPTION OF THE INVENTION

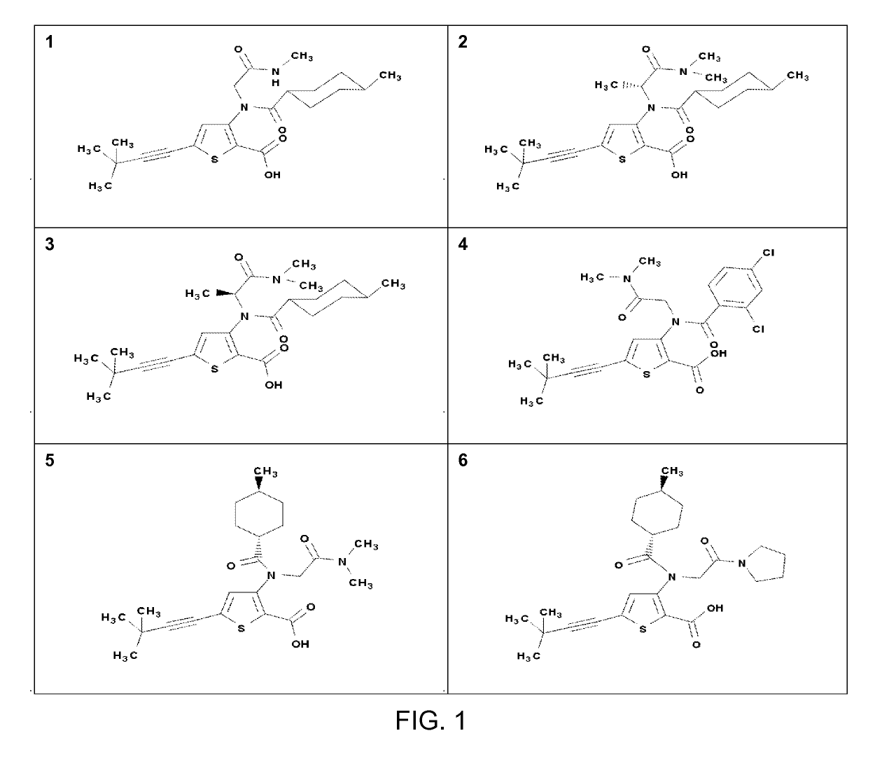

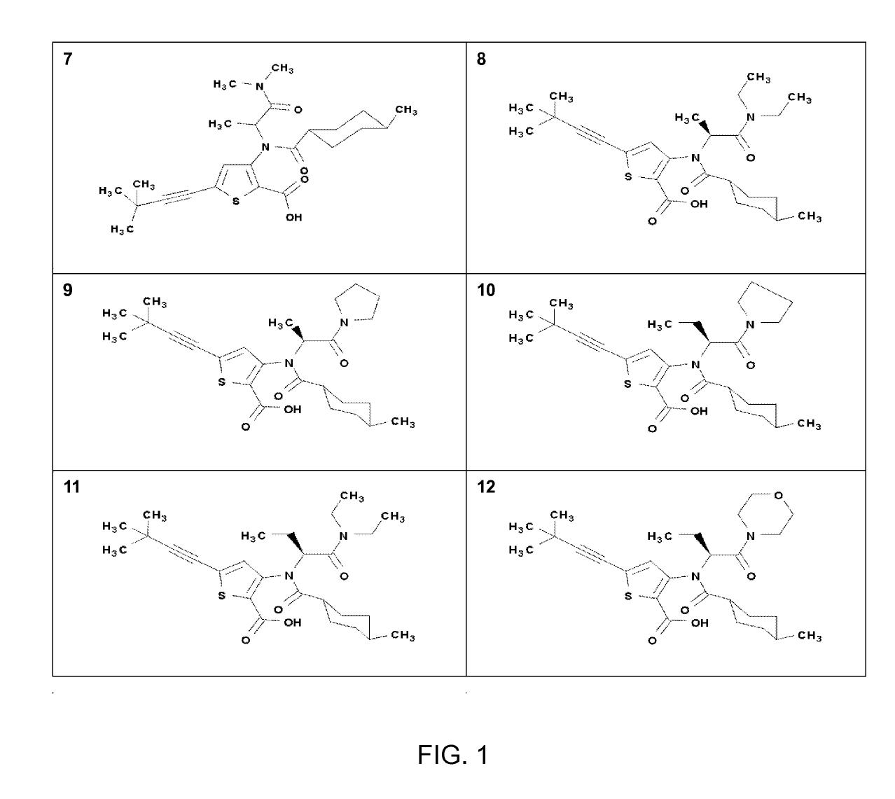

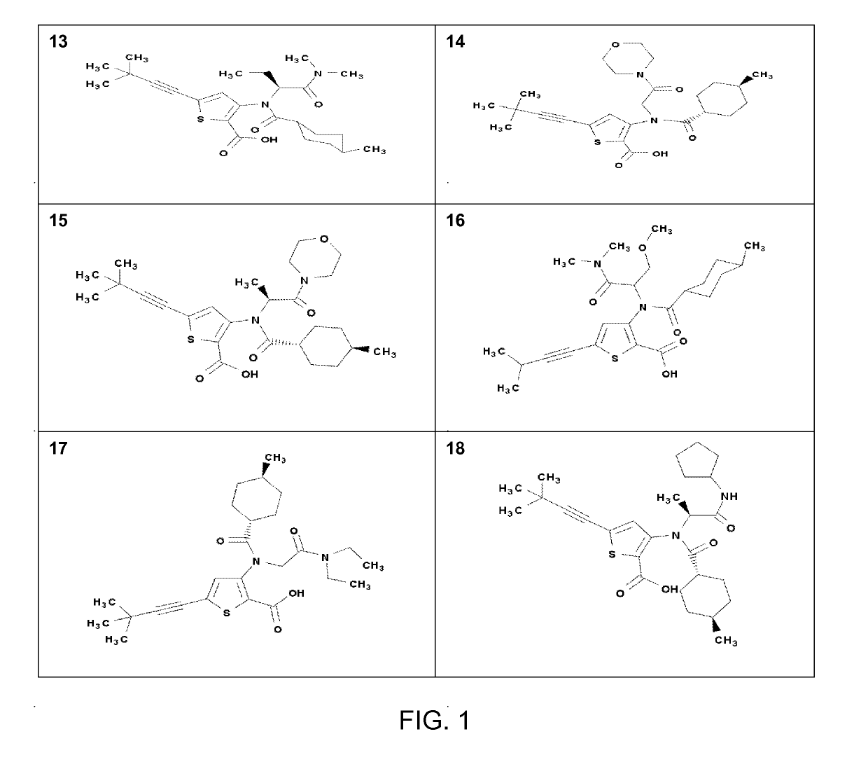

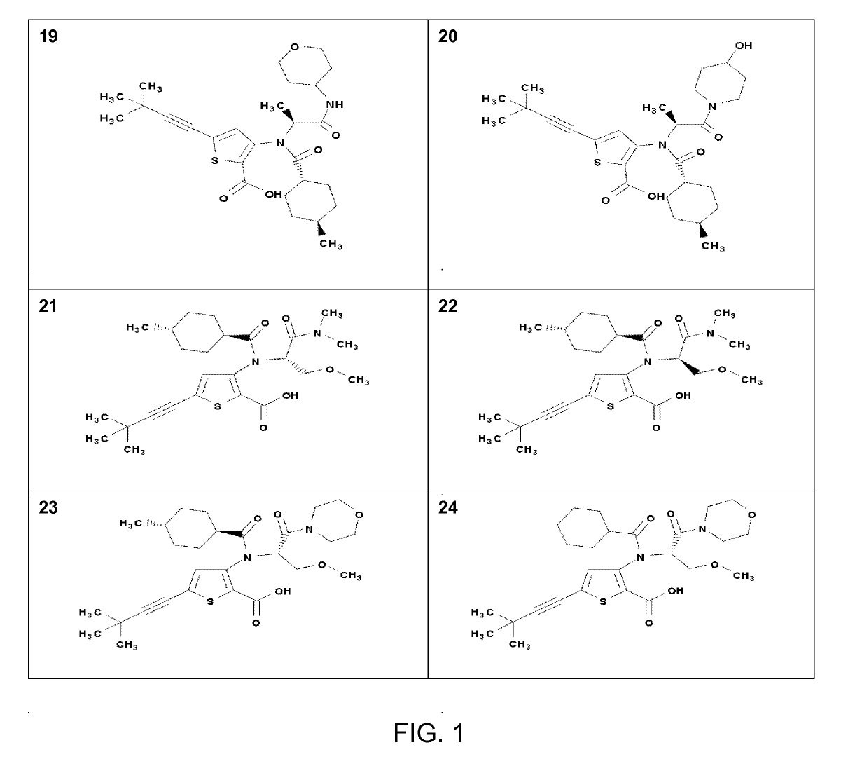

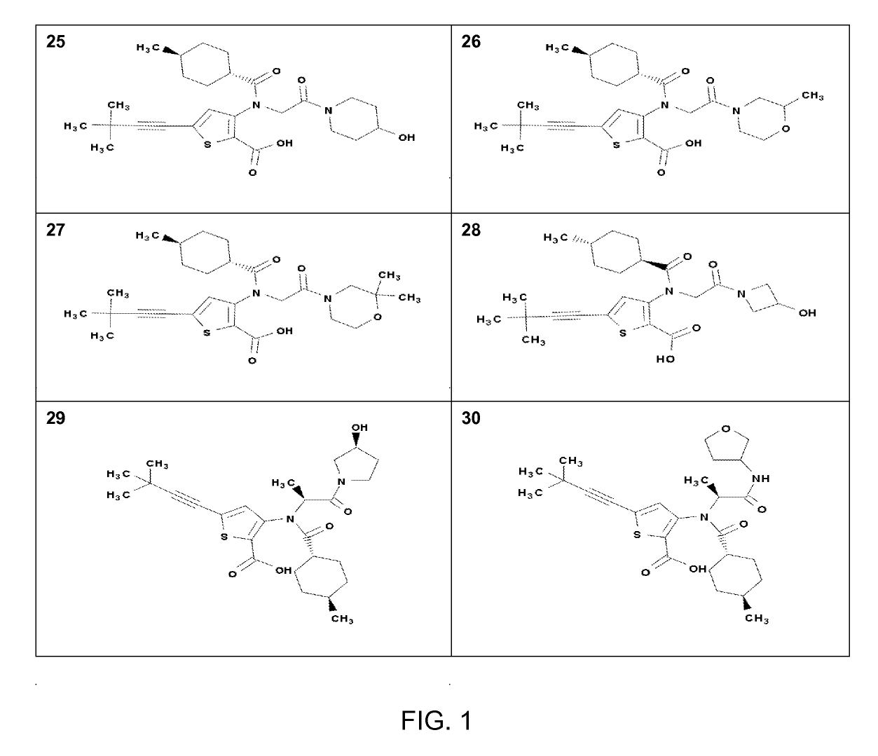

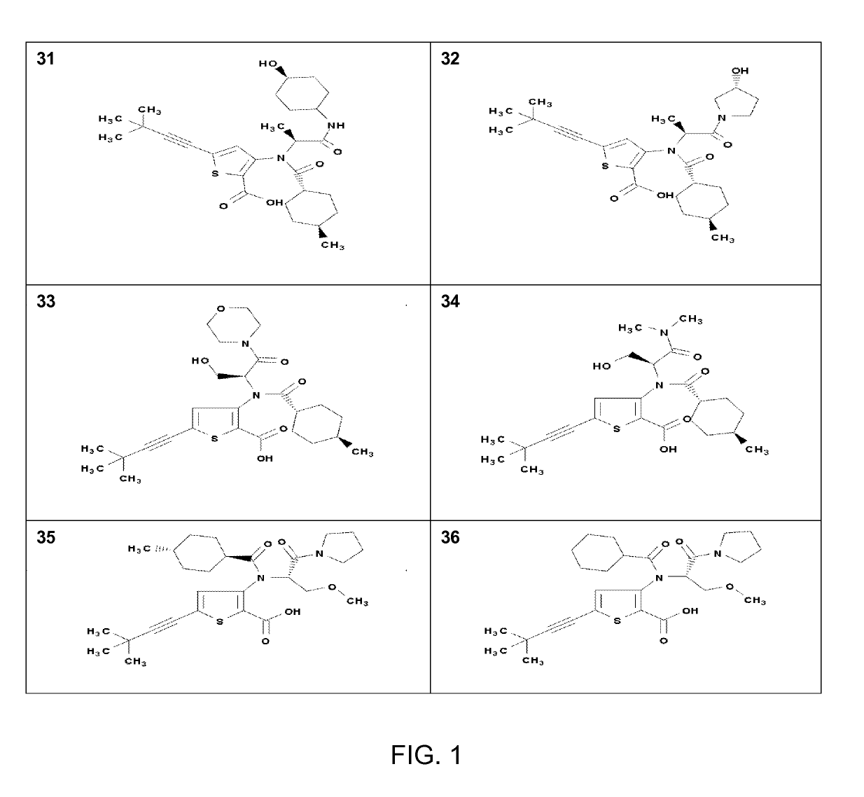

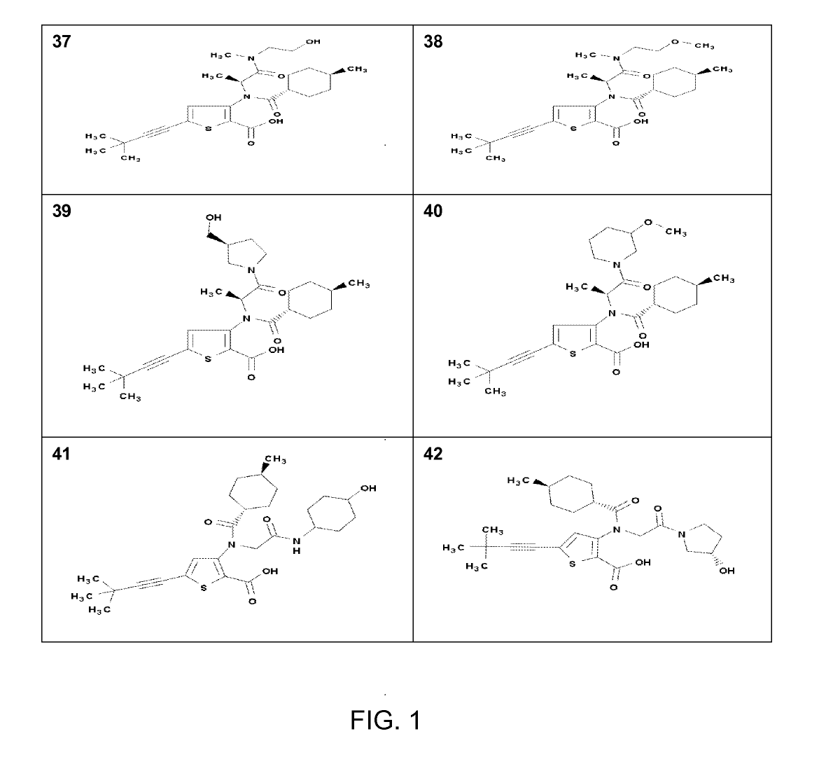

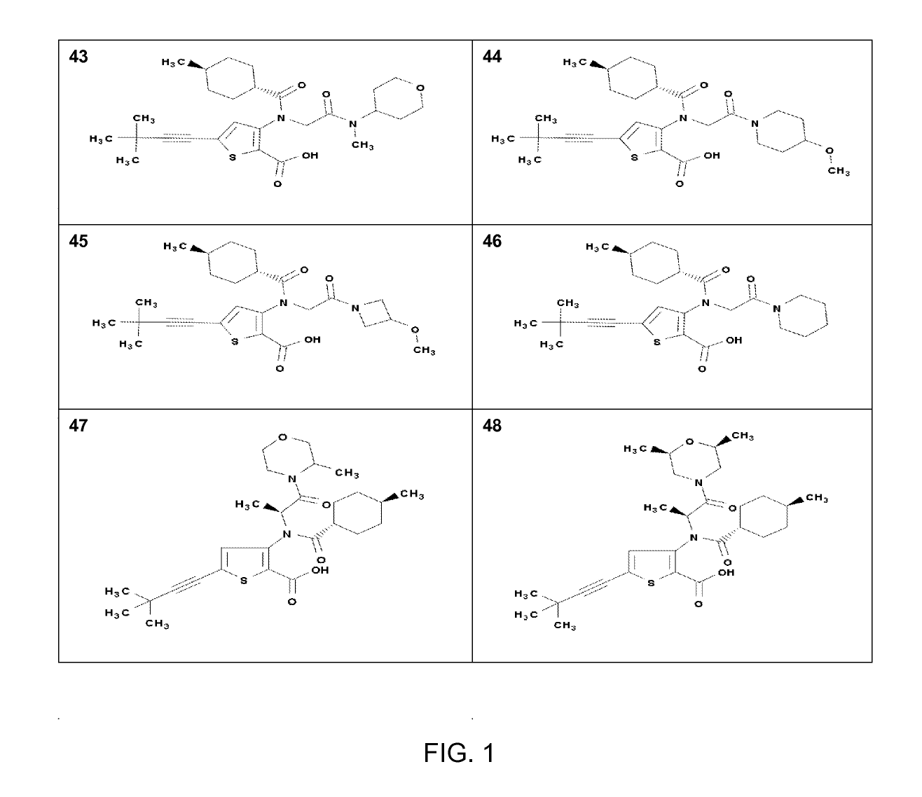

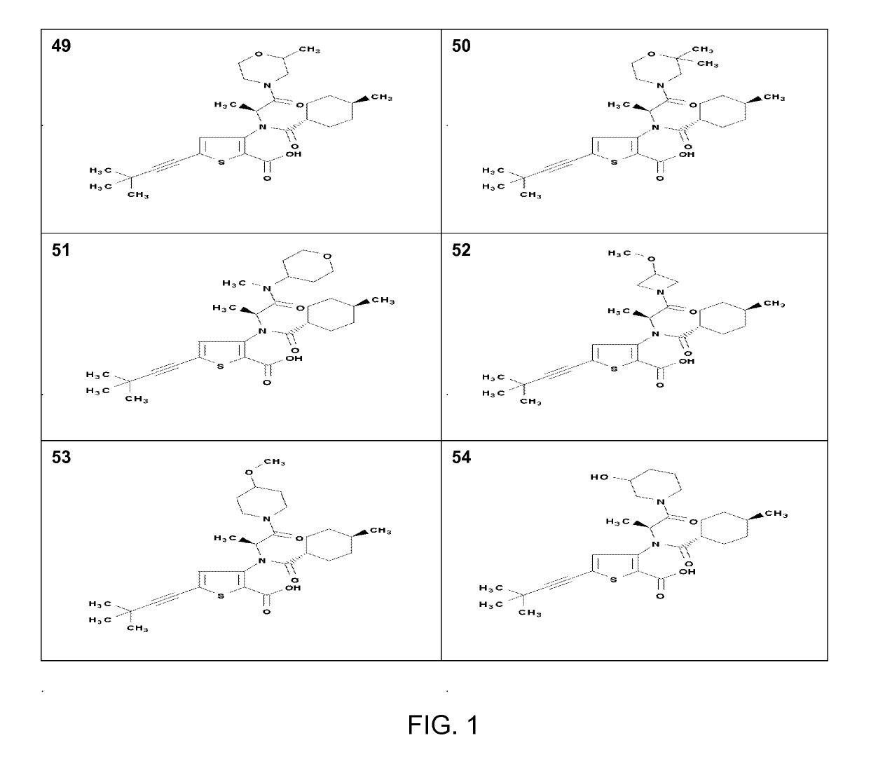

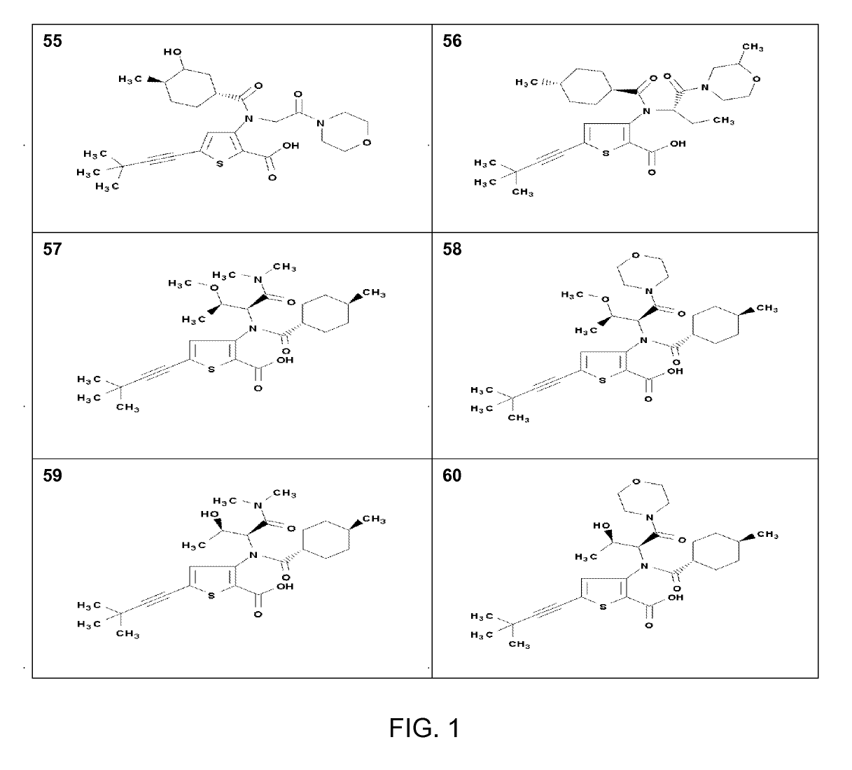

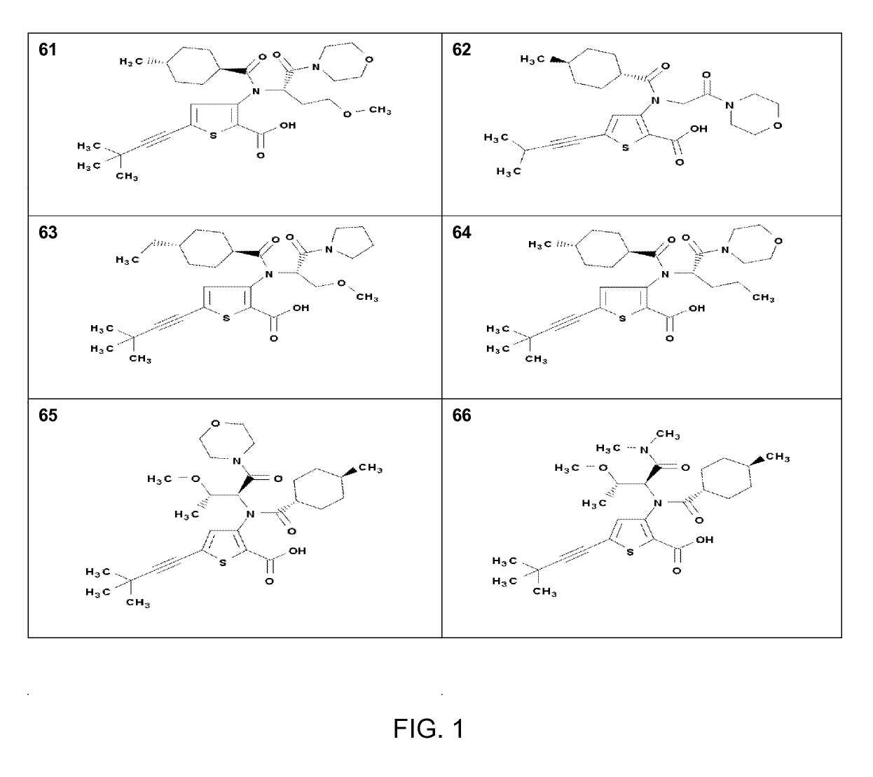

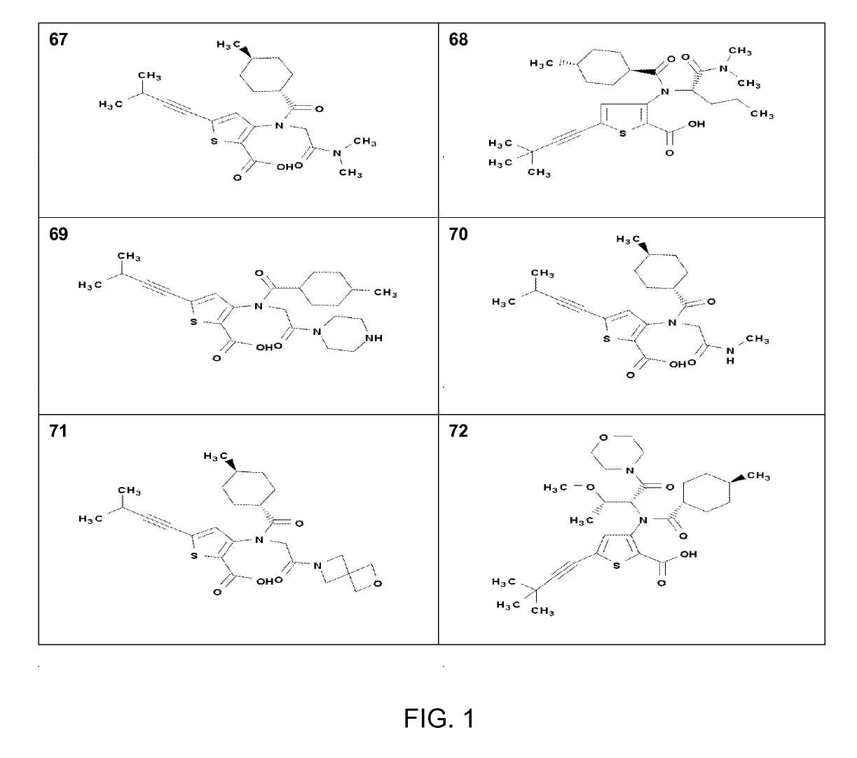

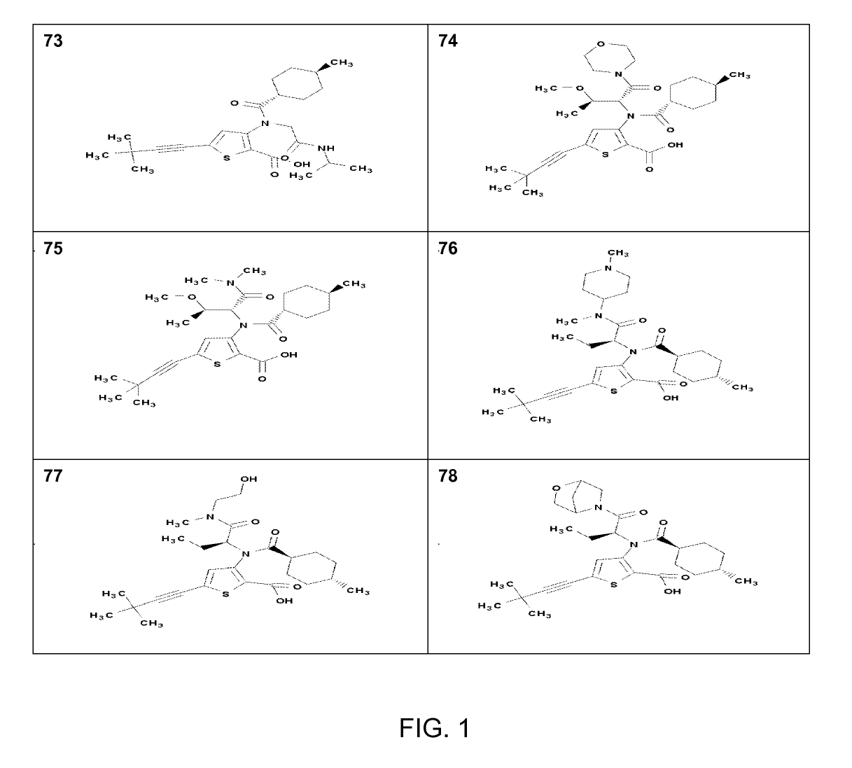

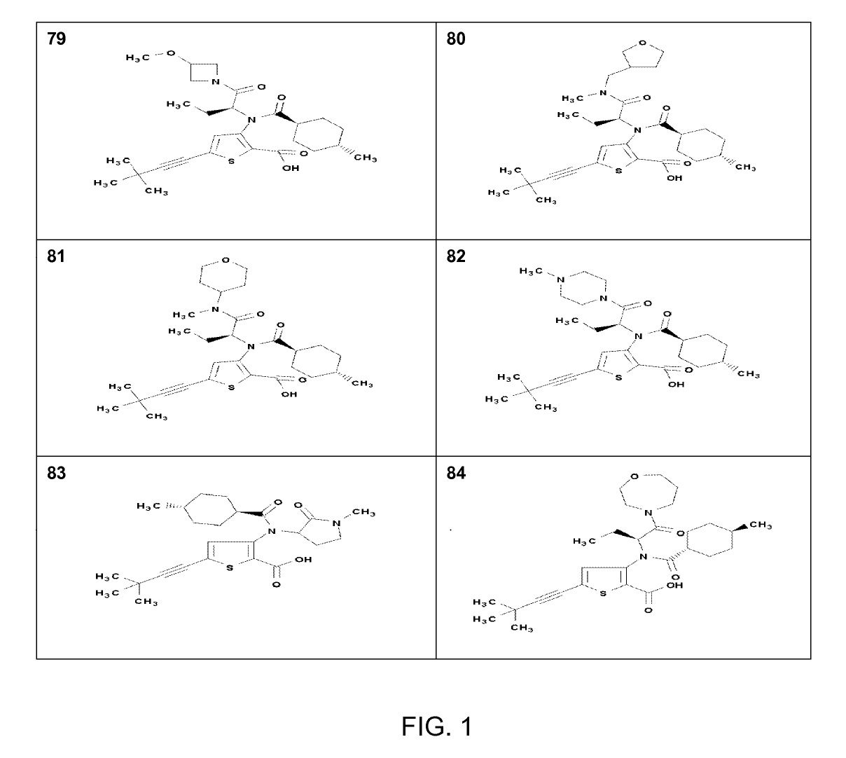

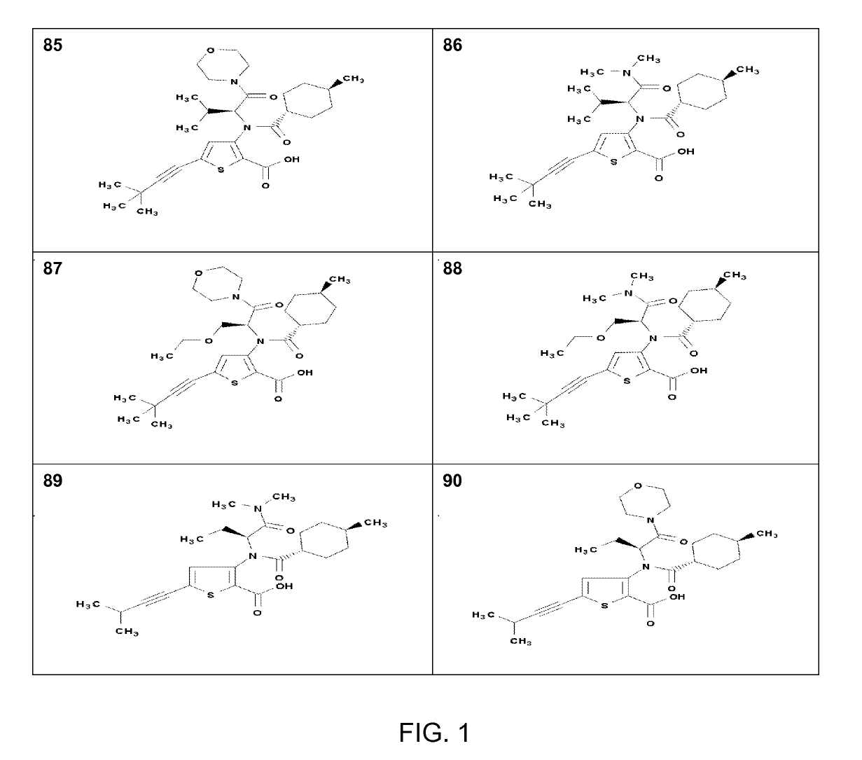

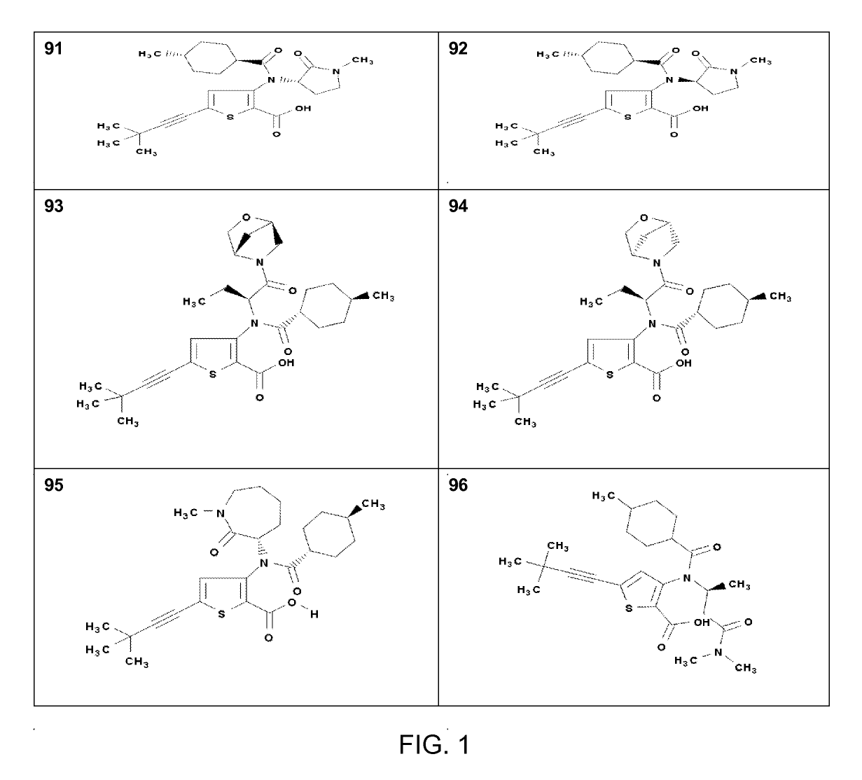

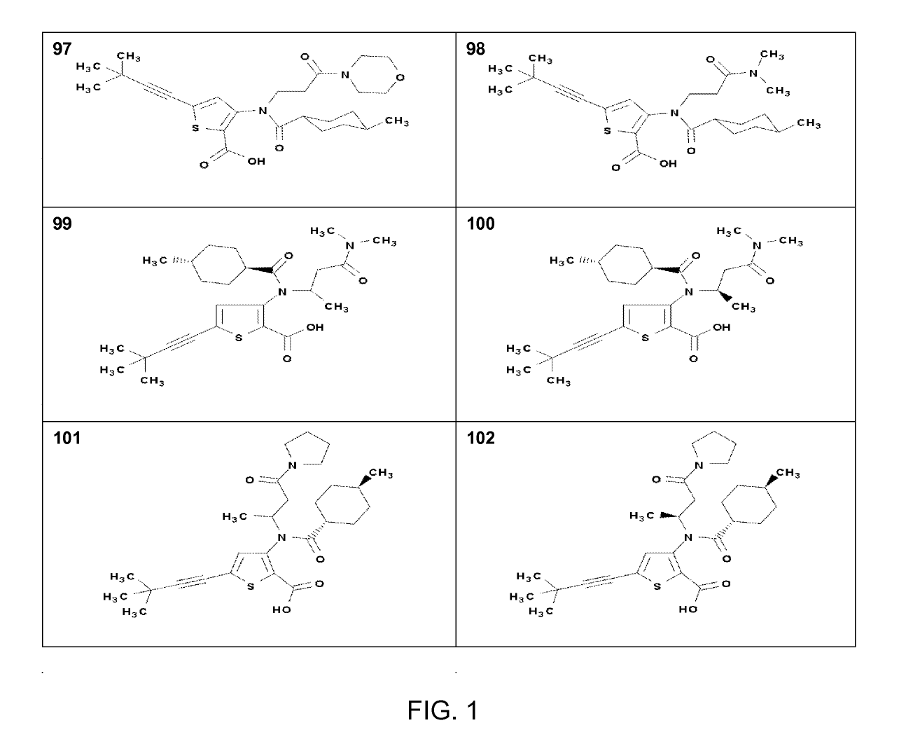

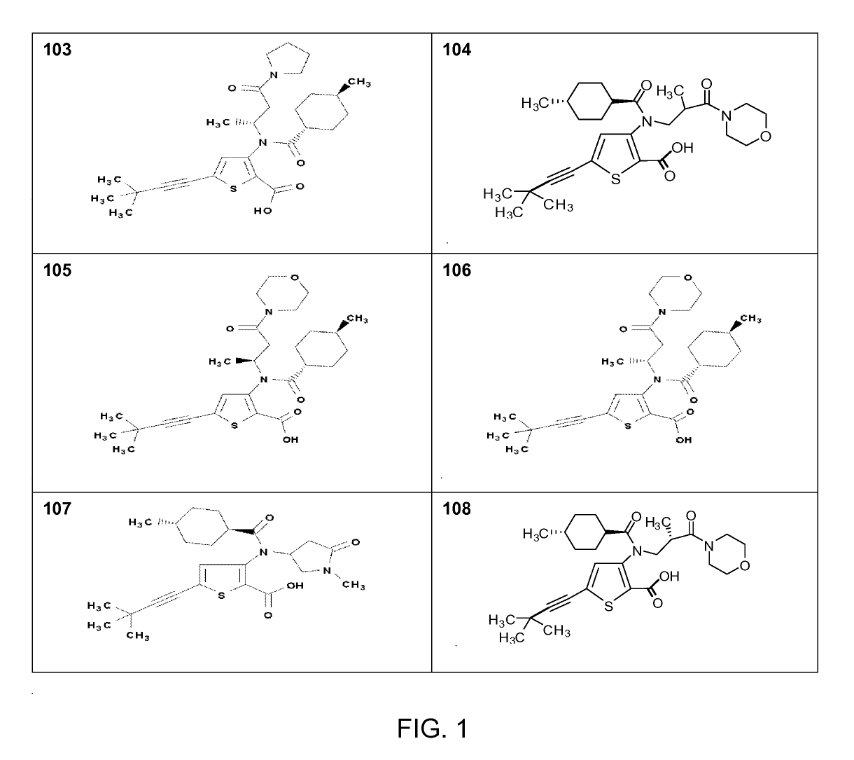

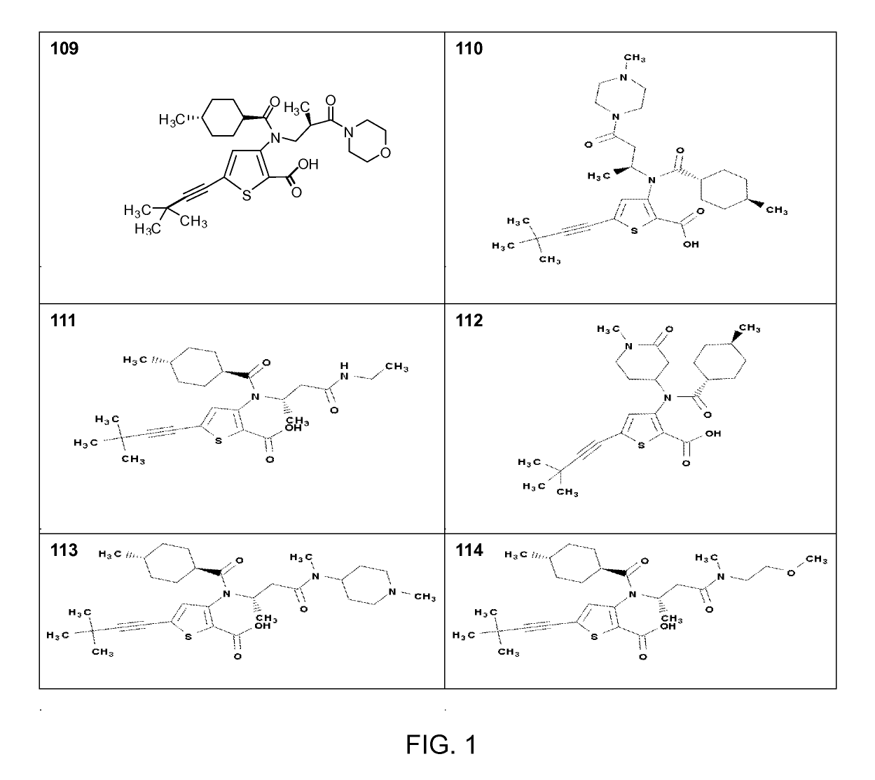

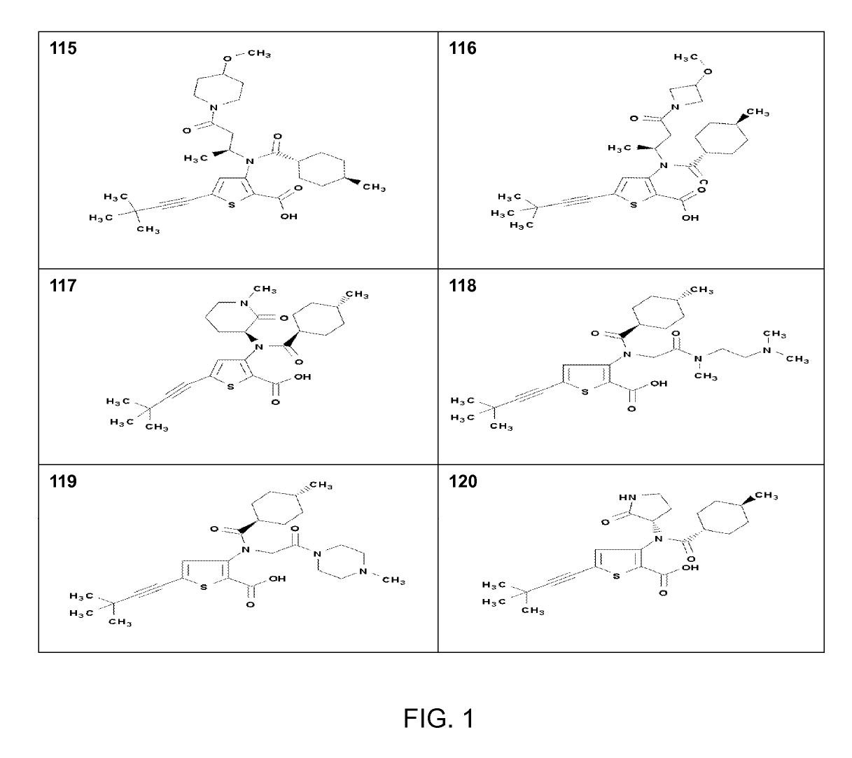

The compounds of the invention are as described in the claims. In some embodiments, the compounds of the invention are represented by any one of Structural Formulae (I)-(XIII) (XX), and (XXI), or pharmaceutically acceptable salts thereof, wherein the variables are each and independently as described in any one of the claims. In some embodiments, the compounds of the invention are represented by any chemical formulae depicted in FIGS. 1, 2, and 3, or pharmaceutically acceptable salts thereof. In some embodiments, the compounds of the invention are presented by any one of Structural Formulae (I)-(XIII), (XX), and (XXI), or pharmaceutically acceptable salts thereof, wherein the variables are each and independently as depicted in the chemical formulae in FIGS. 1, 2, and 3.

In one embodiment, the compounds of the invention are represented by Structural Formula (I):

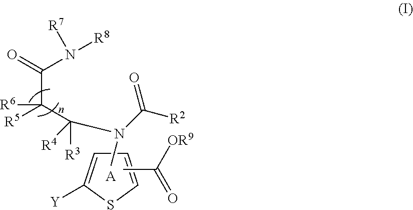

or pharmaceutically acceptable salts thereof. A first set of values of the variables of Structural Formula (I) is as follows:

Ring A is optionally further substituted with one or more substituents selected from the group consisting of -D, halogen, —CN, C₁₋₆ alkyl, and C₁₋₆ haloalkyl. In one aspect, ring A is optionally further substituted with —F. Alternatively, ring A is not further substituted.

Y is C₃₋₈ carbocycle, 5-8 membered heterocycle, —(C₂ aliphatic group)-R¹, C₆₋₁₀ aryl, or 5-10 membered heteroaryl, wherein each of said carbocycle, heterocycle, aryl and heteroaryl is optionally and independently substituted with one or more instances of J^{Y} and wherein said C₂ aliphatic group is optionally substituted with one or more substituents selected from the group consisting of halogen, —CN, C₁₋₂ alkyl, C₁₋₂ haloalkyl, hydroxy, and methoxy. Generally, Y is optionally substituted C₃₋₆cycloalkyl, optionally substituted C₄₋₆cycloalkenyl, —(C₂ aliphatic group)-R¹, optionally substituted phenyl, or optionally substituted 5-6 membered heteroaryl, and wherein said C₂ aliphatic group is optionally substituted. In one aspect, Y is —(C₂ aliphatic group)-R¹ or optionally substituted phenyl, wherein said C₂ aliphatic group is optionally substituted. In another aspect, Y is optionally substituted phenyl, optionally substituted thienyl, or optionally substituted pyridyl. Specifically Y is phenyl. In yet another aspect, Y is optionally substituted C₃₋₆cycloalkyl or optionally substituted C₄₋₆cycloalkenyl. In yet another aspect, Y is optionally substituted C₄₋₆cycloalkenyl. In yet another aspect, Y is optionally substituted cyclohexenyl. In yet another aspect, Y is —(C₂ aliphatic group)-R¹, and wherein said C₂ aliphatic group is optionally substituted. In yet another aspect, Y is —CH₂—CH₂—R¹, —CH═CH—R¹, or —C≡CR¹.

R¹ is i) —H; ii) a C₁₋₆ alkyl group optionally substituted with one or more instances of J^{1A}; iii) a C₃₋₁₀ carbocycle or 4-10 membered heterocycle, each of which is optionally and independently substituted with one or more instances of J^{1B}; or iv) a C₆₋₁₀ aryl or 5-10 membered heteroaryl group, each of which is optionally and independently substituted with one or more instances of J^{1C}. In one aspect, R¹ is optionally substituted C₁₋₆ alkyl, optionally substituted C₃₋₈ carbocyclic, optionally substituted phenyl, or optionally substituted 5-6 membered heteroaryl. In another aspect, R¹ is an optionally substituted C₁₋₆alkyl or optionally substituted C₃₋₈ carbocyclic group. In yet another aspect, R¹ is an optionally substituted C₁₋₆ alkyl or C₃₋₈cycloalkyl. In yet another aspect, R¹ is an optionally substituted C₁₋₆ alkyl. In yet another aspect, R¹ is optionally substituted t-butyl or isopropyl. In yet another aspect, R¹ is optionally substituted cyclopropyl.

R² is i) a C₁₋₆ aliphatic group optionally substituted with one or more instances of J^{2A}; ii) a C₃₋₁₀ carbocycle or 4-10 membered heterocycle, each of which is optionally and independently substituted with one or more instances of J^{2B}; or iii) a C₆₋₁₀ aryl or 5-10 membered heteroaryl group, each of which is optionally and independently substituted with one or more instances of J^{2C}. In one aspect, R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group. In another aspect, R² is optionally substituted C₅-C₈ cycloalkyl or optionally substituted phenyl. In yet another aspect, R² is optionally substituted C₅-C₈ cycloalkyl. In yet another aspect, R² is optionally substituted cyclohexyl.

Each of R³, R⁴, R⁵ and R⁶ independently is —H, -D, or a C₁₋₆ aliphatic group optionally substituted with one or more instances of J^{D}. Optionally, when Y is —(C₂ aliphatic group)-R¹, R³ and R⁷, together with the atoms to which they are attached, form a 4-10 membered heterocyclic ring optionally substituted with one or more instances of J^{E}.

In one aspect, each of R³, R⁴, R⁵ and R⁶ independently is —H or an optionally substituted C₁₋₆ alkyl group; or optionally, when Y is —(C₂ aliphatic group)-R¹, R³, together with R⁷ and the atom to which it is attached, forms an optionally substituted, 4-membered heterocyclic ring. In another aspect, each of R³, R⁴, R⁵ and R⁶ independently is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl; or optionally, when Y is —(C₂ aliphatic group)-R¹, R³, together with R⁷ and the atom to which it is attached, forms a 4-10 membered heterocyclic ring optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In yet another aspect, each of R³, R⁴, R⁵ and R⁶ independently is —H or C₁₋₆alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆ haloalkyl); or optionally, when Y is —(C₂ aliphatic group)-R¹, R³, together with R⁷ and the atom to which it is attached, forms a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆ haloalkyl).

Examples of the heterocyclic ring formed with R³ and R⁷ include:

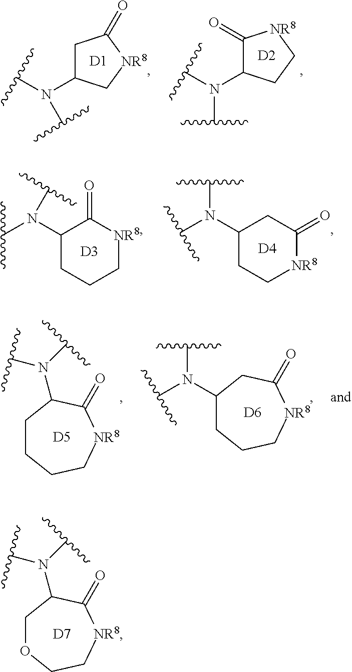

wherein each of rings D1-D7 is independently and optionally further substituted.

Each of R⁷ and R⁸ independently is i) —H; ii) a C₁₋₆ aliphatic group optionally substituted with one or more instances of J^{7A}; iii) a C₃₋₁₀ carbocycle or 4-10 membered heterocycle, each of which is optionally and independently substituted with one or more instances of J^{7B}; or iv) a C₆₋₁₀ aryl or 5-10 membered heteroaryl group, each of which is optionally and independently substituted with one or more instances of J^{7C}; or R⁷ and R⁸, together with the nitrogen atom to which they are attached, form a 4-10 membered heterocyclic ring optionally substituted with one or more instances of J^{E}.

In one aspect, each of R⁷ and R⁸ independently is an optionally substituted C₁₋₆ aliphatic, optionally substituted C₃₋₈ carbocyclic, or optionally substituted, 4-8 membered heterocyclic group, or R⁷ and R⁸, together with the nitrogen atom to which they are attached, form an optionally substituted, 4-10 membered heterocyclic ring. In another aspect, each of R⁷ and R⁸ independently is —H or an optionally substituted C₁₋₆alkyl, optionally substituted C₃₋₈ carbocyclic, or optionally substituted 4-8 membered heterocyclic group; or R⁷ and R⁸, together with the atom to which they are attached, optionally form an optionally substituted, 4-10 membered heterocyclic ring. In yet another aspect, each of R⁷ and R⁸ independently is —H; C₁₋₆alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; or a C₃₋₈ carbocyclic or 4-8 membered heterocyclic group each of which optionally and independently substituted with one or more substituents selected from the group consisting of halogen,
oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl; or R⁷ and R⁸, together with the atom to which they are attached, optionally form a 4-10 membered heterocyclic ring optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen,
oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl. In yet another aspect, each of R⁷ and R⁸ independently is —H or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl); or R⁷ and R⁸, together with the atom to which they are attached, optionally form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆ haloalkyl). In yet another aspect, R⁷ and R⁸, together with the atom to which they are attached, form an optionally substituted heterocyclic ring. The heterocyclic ring formed with R⁷ and R⁸ can be a bridged or spiro ring.

R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}. In one aspect, R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In another aspect, R⁹ is —H or C₁₋₆alkyl. In yet another aspect, R⁹ is —H.

Each J^{Y} is independently selected from the group consisting of halogen, —CN, nitro, azido, R^{a}, —SO₂R^{a}, —OR^{a}, —COR^{a}, —NRR^{a}, —C(O)OR^{a}, —OC(O)R^{a}, —NRC(O)R^{a}, —C(O)NRR^{a}, —NRC(O)NRR^{a}, —NRC(O)OR^{a}, —OCONRR^{a}, —SO₂NRR^{a}, —NRSO₂R^{a}, —NRSO₂NRR^{a}, and —NRC(═NR)NRR^{a}. Typical examples of J^{Y} include halogen, —CN, nitro, R^{a}, —OR^{a}, —COR^{a}, and —NRR^{a}. More typical examples of J^{Y} include halogen, —CN, nitro, C₁₋₆alkyl, C₁₋₆haloalkyl, —OH, —O(C₁₋₆ alkyl), —O(phenyl), —O(5-6 membered heteroaryl), —NH₂, —NH(C₁₋₆ alkyl), —N(C₁₋₆ alkyl)₂, and —C(O)(C₁₋₆alkyl). More typical examples of J^{Y} include halogen, —CN, nitro, C₁₋₆ alkyl, C₁₋₆haloalkyl, —OH, —O(C₁₋₆alkyl), —NH₂, —NH(C₁₋₆ alkyl), —N(C₁₋₆ alkyl)₂, and —C(O)(C₁₋₆ alkyl). More typical examples of J^{Y} include halogen, —CN, nitro, methyl, ethyl, —CF₃, —OH, —OMe, —NH₂, and —C(O)Me.

Each of J^{1A}, J^{2A}, J^{7A}, and J^{9A} independently is oxo or Q; or two J^{1A}, two J^{2A}, two J^{7A}, and two J^{9A}, respectively, together with the atom(s) to which they are attached, optionally and independently form a 3-8-membered non-aromatic ring that is optionally substituted with one or more instances of J^{E}. Typical examples of J^{1A}, J^{2A}, and J^{7A} independently include halogen,

oxo, —CN, —OR^{a}, —NRR^{a}, —OC(O)R^{a}, —OC(O)OR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈ cyclo(haloalkyl), 5-6 membered optionally substituted heterocyclyl, and optionally substituted phenyl. More typical examples of J^{1A}, J^{2A}, and J^{7A} independently include halogen, oxo, —CN, —OR^{a}, —NRR^{a}, —OC(O)R^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl. Typical examples of J^{9A} include halogen,
oxo, —CN, —OR^{a}, —NRR^{a}, —OC(O)R^{a}, —OC(O)OR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈ cyclo(haloalkyl), phenyl, and 5-6 membered heterocyle optionally substituted with one or more substituents selected from oxo and C₁₋₆alkyl. More typical examples of J^{9A} include —OC(O)R^{a}, —OC(O)OR^{a}, phenyl, and 5-6 membered heterocyle optionally substituted with one or more substituents selected from oxo and C₁₋₆ alkyl. More typical examples of J^{9A} include include halogen,
oxo, —CN, —OR^{a}, —NRR^{a}, —OC(O)R^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl.

Each of J^{1B}, J^{2B}, J^{7B}, and J^{9B} and independently is oxo, Q, or a C₁₋₆ aliphatic group optionally substituted with one or more instances of Q; or two J^{1B}, two J^{2B}, two J^{3B}, two J^{1B}, and two J^{9B}, respectively, together with the atom(s) to which they are attached, optionally and independently form a 3-8-membered non-aromatic ring that is optionally substituted with one or more instances of J^{E}. Typical examples of J^{1B}, J^{2B}, J^{7B}, and J^{9B} independently include halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, and a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl. More typical examples of J^{1B}, J^{2B}, J^{7B}, and J^{9B} independently include halogen, —CN, —OH, —NH₂, —NH(C₁₋₆ alkyl), —N(C₁₋₆ alkyl)₂, —O(C₁₋₆ alkyl), and C₁₋₆ alkyl optionally substituted with one or more substituents selected from the group consisting of halogen, —CN, —OH, —NH₂, —NH(C₁₋₆ alkyl), —N(C₁₋₆alkyl)₂, —O(C₁₋₆ alkyl), and —O(C₁₋₆haloalkyl).

Each of J^{1C}, J^{2C}, J^{7C} and J^{9C} independently is Q or a C₁₋₆ aliphatic group optionally substituted with one or more instances of Q; or two J^{1C}, two J^{2C}, two J^{7C}, and two J^{9C}, respectively, together with the atom(s) to which they are attached, optionally and independently form a 3-8-membered non-aromatic ring that is optionally substituted with one or more instances of J^{E}. Typical examples of J^{1C}, J^{2C}, J^{7C}, and J^{9C} independently include halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, and a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Typical examples of Q include halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —OC(O)OR^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Alternatively, typical examples of Q include halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl.

Each R^{a} independently is: i) —H; ii) a C₁₋₆ aliphatic group optionally substituted with one or more substituents independently selected from the group consisting of halogen, oxo, —CN, —OR, —NRR′, —OCOR, —COR″, —CO₂R, —CONRR′, —NRC(O)R, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or R^{a}, together with R and the nitrogen atom to which it is attached, optionally forms a 4-8 membered heterocycle optionally substituted with one or more instances of J^{E}. In one aspect, R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring. In another aspect, R^{a} is —H, optionally substituted C₁₋₆alkyl, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring.

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or R′, together with R and the nitrogen atom to which it is attached, optionally forms a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.

Each J^{E} is independently selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of

halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

The value of n is 0 or 1. Typically n is 0.

A second set of values of the variables of Structural Formula (I) is as follows:

Ring A is optionally further substituted with —F.

The remaining variables of Structural Formula (I) are each and independently as described above in the first set of values of the variables of Structural Formula (I).

A third set of values of the variables of Structural Formula (I) is as follows:

R¹ is an optionally substituted C₁₋₆ alkyl or optionally substituted C₃₋₈ carbocyclic group. Suitable substituents for R¹ are as described above in the first set of variables of Structural Formula (I). In one aspect, R¹ is an optionally substituted C₁₋₆alkyl or C₃₋₈ cycloalkyl, each of which is optionally and independently substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.
In another aspect, R¹ is C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In yet another aspect, R¹ is C₁₋₆ alkyl optionally substituted with one or more substituents selected from the group consisting of halogen, —CN, —OH, and —O(C₁-C₆ alkyl).

The remaining variables of Structural Formula (I) are each and independently as described above in the first set of variables of Structural Formula (I).

A fourth set of values of the variables of Structural Formula (I) is as follows:

R¹ is as described above in any one of the first through third sets of values of the variables of Structural Formula (I).

Each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —OC(O)R^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Alternatively, Each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Suitable substituents for the C₃₋₈ carbocyclic, heterocyclyl, phenyl, and heteroaryl groups are each and independently as described above in the first set of variables of Structural Formula (I).

The remaining variables of Structural Formula (I) are each and independently as described above in the first set of values of the variables of Structural Formula (I).

A fifth set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring.

The remaining variables of Structural Formula (I) are each and independently as described above in the first set of values of the variables of Structural Formula (I).

A sixth set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, and J^{9A} independently is halogen,

oxo, —CN, —OR^{a}, —NRR^{a}, —OC(O)R^{a}, —OC(O)OR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈ cyclo(haloalkyl), 5-6 membered optionally substituted heterocyclyl, or optionally substituted phenyl. Alternatively, each of J^{1A}, J^{2A}, J^{7A}, and J^{9A} independently is halogen, oxo, —CN, —OR^{a}, —NRR^{a}, —OC(O)R^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), or phenyl.

Each of J^{1B}, J^{2B}, J^{7B}, and J^{9B} independently is halogen,

oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl.

Each of J^{1C}, J^{2C}, J^{7C}, and J^{9C} independently is halogen,

oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl.

The remaining variables of Structural Formula (I) are each and independently as described above in the first set of values of the variables of Structural Formula (I).

A seventh set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, J^{9A}, J^{1B}, J^{2B}, J^{7B}, J^{9B}, J^{1C}, J^{2C}, J^{7C}, and J^{9C} independently is as described above in any one of the first through sixth sets of values of the variables of Structural Formula (I).

R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group. In one aspect, R² is optionally substituted C₅-C₈ cycloalkyl or optionally substituted phenyl. Suitable substituents for the C₁₋₆ aliphatic, C₃₋₈ carbocyclic, heterocyclyl, phenyl, and heteroaryl groups are each and independently as described above in the first set of variables of Structural Formula (I). In another aspect, R² is C₅-C₈ cycloalkyl optionally substituted with one or more substituents selected from the group consisting of halogen;

oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In yet another aspect, R² is cyclohexyl optionally substituted with one or more substituents selected from the group consisting of halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl. In yet another aspect, R² is cyclohexyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and C₁₋₆alkyl optionally substituted with one or more substituents selected from the group consisting of halogen, —CN, —OH, —NH₂, —NH(C₁₋₆alkyl), —N(C₁₋₆alkyl)₂, —O(C₁₋₆alkyl), and —O(C₁₋₆ haloalkyl).

The remaining variables of Structural Formula (I) are each and independently as described above in the first set of values of the variables of Structural Formula (I).

An eighth set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, J^{9A}, J^{1B}, J^{2B}, J^{7B}, J^{9B}, J^{1C}, J^{2C}, J^{7C}, and J^{9C} independently is as described above in any one of the first through sixth sets of values of the variables of Structural Formula (I).

R² is as described above in any one of the first through seventh sets of values of the variables of Structural Formula (I).

Each J^{Y} is independently selected from the group consisting of halogen, —CN, nitro, R^{a}, —OR^{a}, —COR^{a}, and —NRR^{a}. In one aspect, each J^{Y} is independently selected from the group consisting of halogen, —CN, nitro, C₁₋₆alkyl, C₁₋₆haloalkyl, —OH, —O(C₁₋₆ alkyl), —O(phenyl), —O(5-6 membered heteroaryl), —NH₂, —NH(C₁₋₆ alkyl), —N(C₁₋₆ alkyl)₂, and —C(O)(C₁₋₆ alkyl). In another aspect, each J^{Y} is independently selected from the group consisting of halogen, —CN, nitro, C₁₋₆alkyl, C₁₋₆haloalkyl, —OH, —O(C₁₋₆ alkyl), —NH₂, —NH(C₁₋₆ alkyl), —N(C₁₋₆ alkyl)₂, and —C(O)(C₁₋₆alkyl). In yet another aspect, each J^{Y} is independently selected from the group consisting of halogen, —CN, nitro, methyl, ethyl, —CF₃, —OH, —OMe, —NH₂, and —C(O)Me.

The remaining variables of Structural Formula (I) are each and independently as described above in the first set of values of the variables of Structural Formula (I).

A ninth set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, J^{9A}, J^{1B}, J^{2B}, J^{7B}, J^{9B}, J^{1C}, J^{2C}, J^{7C}, and J^{9C} independently is as described above in any one of the first through sixth sets of values of the variables of Structural Formula (I).

R² is as described above in any one of the first through seventh sets of values of the variables of Structural Formula (I).

Each J^{Y} is independently as described above in any one of the first through eighth sets of values of the variables of Structural Formula (I).

Y is optionally substituted C₃₋₆cycloalkyl, optionally substituted C₄₋₆ cycloalkenyl, —(C₂ aliphatic group)-R¹, optionally substituted phenyl, or optionally substituted 5-6 membered heteroaryl, and wherein said C₂ aliphatic group is optionally substituted. In one aspect, Y is —(C₂ aliphatic group)-R¹ or optionally substituted phenyl, wherein said C₂ aliphatic group is optionally substituted. In another aspect, Y is optionally substituted phenyl, optionally substituted thienyl, or optionally substituted pyridyl. In yet another aspect, Y is optionally substituted phenyl. Suitable substituents for the values of Y are as described above in the first set of variables of Structural Formula (I).

The remaining variables of Structural Formula (I) are each and independently as described above in the first set of values of the variables of Structural Formula (I).

A tenth set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, J^{9A}, J^{1B}, J^{2B}, J^{7B}, J^{9B}, J^{2C}, J^{7C}, and J^{9C} independently is as described above in any one of the first through sixth sets of values of the variables of Structural Formula (I).

R² is as described above in any one of the first through seventh sets of values of the variables of Structural Formula (I).

Each J^{Y} is independently as described above in any one of the first through eighth sets of values of the variables of Structural Formula (I).

Y is optionally substituted C₃₋₆cycloalkyl or optionally substituted C₄₋₆ cycloalkenyl. In one aspect, Y is optionally substituted C₄₋₆cycloalkenyl. In another aspect, Y is optionally substituted cyclohexenyl. Suitable substituents for the values of Y are as described above in the first set of variables of Structural Formula (I).

The remaining variables of Structural Formula (I) are each and independently as described above in the first set of values of the variables of Structural Formula (I).

An eleventh set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, J^{9A}, J^{1B}, J^{2B}, J^{7B}, J^{9B}, J^{2C}, J^{7C}, and J^{9C} independently is as described above in any one of the first through sixth sets of values of the variables of Structural Formula (I).

R² is as described above in any one of the first through seventh sets of values of the variables of Structural Formula (I).

Y is as described above in any one of the first, ninth and tenth sets of values of the variables of Structural Formula (I).

Each J^{Y} is independently selected from the group consisting of halogen, —CN, nitro, R^{a}, —OR^{a}, —COR^{a}, and —NRR^{a}. In one aspect, each J^{Y} is independently selected from the group consisting of halogen, —CN, nitro, C₁₋₆alkyl, C₁₋₆haloalkyl, —OH, —O(C₁₋₆ alkyl), —O(phenyl), —O(5-6 membered heteroaryl), —NH₂, —NH(C₁₋₆ alkyl), —N(C₁₋₆ alkyl)₂, and —C(O)(C₁₋₆ alkyl). In another aspect, each J^{Y} is independently selected from the group consisting of halogen, —CN, nitro, C₁₋₆alkyl, C₁₋₆haloalkyl, —OH, —O(C₁₋₆ alkyl), —NH₂, —NH(C₁₋₆ alkyl), —N(C₁₋₆alkyl)₂, and —C(O)(C₁₋₆alkyl). In yet another aspect, each J^{Y} is independently selected from the group consisting of halogen, —CN, nitro, methyl, ethyl, —CF₃, —OH, —OMe, —NH₂, and —C(O)Me.

The remaining variables of Structural Formula (I) are each and independently as described above in the first set of values of the variables of Structural Formula (I).

A twelfth set of values of the variables of Structural Formula (I) is as follows:

R¹ is as described above in any one of the first through third sets of values of the variables of Structural Formula (I).

Each Q is independently as described above in any one of the first through fourth sets of values of the variables of Structural Formula (I).

R^{a} is as described above in any one of the first through fifth sets of values of the variables of Structural Formula (I).

Each of J^{1A}, J^{2A}, J^{7A}, J^{9A}, J^{1B}, J^{2B}, J^{7B}, J^{9B}, J^{1C}, J^{2C}, J^{7C}, and J^{9C} independently is as described above in any one of the first through sixth sets of values of the variables of Structural Formula (I).

R² is as described above in any one of the first through seventh sets of values of the variables of Structural Formula (I).

Y is —(C₂ aliphatic group)-R¹, and wherein said C₂ aliphatic group is optionally substituted. In one aspect, Y is —CH₂—CH₂—R¹, —CH═CH—R¹, or —C≡CR¹. Typical examples of substituents suitable for the C₂ aliphatic group of —(C₂ aliphatic group)-R¹ include halogen, —CN, C₁₋₂alkyl, C₁₋₂haloalkyl, hydroxy, and methoxy.

The remaining variables of Structural Formula (I) are each and independently as described above in the first set of values of the variables of Structural Formula (I).

An thirteenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

Each of R³, R⁴, R⁵ and R⁶ independently is —H or an optionally substituted C₁₋₆ alkyl group; or optionally R³, together with R⁷ and the atom to which it is attached, forms an optionally substituted, 4-10 membered heterocyclic ring. Suitable substituents for the values of R³, R⁴, R⁵ and R⁶ are each and independently as described above in the first set of values of the variables of Structural Formula (I).

The remaining variables of Structural Formula (I) are each and independently as described above in the first set of values of the variables of Structural Formula (I).

A fourteenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

Each of R³, R⁴, R⁵ and R⁶ independently is —H or an optionally substituted C₁₋₆ alkyl group; and each of R⁷ and R⁸ independently is an optionally substituted C₁₋₆ aliphatic, optionally substituted C₃₋₈ carbocyclic, or optionally substituted, 4-8 membered heterocyclic group, or R⁷ and R⁸, together with the nitrogen atom to which they are attached, form an optionally substituted, 4-10 membered heterocyclic ring. Suitable substituents for the values of R³, R⁴, R⁵ and R⁶ are each and independently as described above in the first set of values of the variables of Structural Formula (I).

The remaining variables of Structural Formula (I) are each and independently as described above in the first set of values of the variables of Structural Formula (I).

A fifteenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

Each of R³, R⁴, R⁵ and R⁶ independently is —H or C₁₋₆alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; and

each of R⁷ and R⁸ independently is —H; C₁₋₆alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; or a C₃₋₈ carbocyclic or 4-8 membered heterocyclic group each of which optionally and independently substituted with one or more substituents selected from the group consisting of halogen,
oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl; or

R⁷ and R⁸, together with the atom to which they are attached, optionally form a 4-10 membered heterocyclic ring optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; or

R³ and R⁷, together with the atoms to which they are attached, optionally form a 4-10 membered heterocyclic ring optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.

The remaining variables of Structural Formula (I) are each and independently as described above in the first set of values of the variables of Structural Formula (I).

An sixteenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

Each of R³, R⁴, R⁵ and R⁶ independently is —H or optionally substituted C₁₋₆alkyl; and

each of R⁷ and R⁸ independently is —H or an optionally substituted C₁₋₆ alkyl, optionally substituted C₃₋₈ carbocyclic, or optionally substituted 4-8 membered heterocyclic group; or R⁷ and R⁸, together with the atom to which they are attached, optionally form an optionally substituted, 4-10 membered heterocyclic ring. Suitable substituents for the values of R³, R⁴, R⁵, R⁶, R⁷, and R⁸ are each and independently as described above in the fifteenth set of values of the variables of Structural Formula (I).

The remaining variables of Structural Formula (I) are each and independently as described above in the first set of values of the variables of Structural Formula (I).

A seventeenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

Each of R³, R⁴, R⁵ and R⁶ independently is —H or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆ haloalkyl); and

R⁷ and R⁸ are each independently —H or C₁₋₆alkyl optionally substituted with one or more substituents selected from the group consisting of

halogen, —CN, —OH, —NH₂, —NH(C₁₋₆alkyl), —N(C₁₋₆alkyl)₂, —O(C₁₋₆alkyl), and —O(C₁₋₆ haloalkyl); or

R⁷ and R⁸, together with the atom to which they are attached, optionally form a 4-membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl).

The remaining variables of Structural Formula (I) are each and independently as described above in the first set of values of the variables of Structural Formula (I).

An eighteenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

R³, R⁴, R⁵, and R⁶ are each and independently as described above in any one of the thirteenth through seventeenth sets of values of the variables of Structural Formula (I).

R⁷ and R⁸, together with the atom to which they are attached, form a 4-10 membered heterocyclic ring, such as a bridged or spiro ring, which is optionally substituted with one or more substituents selected from the group consisting of halogen, —CN, —OH, —NH₂, —NH(C₁₋₆alkyl), —N(C₁₋₆alkyl)₂, —O(C₁₋₆alkyl), and —O(C₁₋₆ haloalkyl).

The remaining variables of Structural Formula (I) are each and independently as described above in the first set of values of the variables of Structural Formula (I).

A nineteenth set of values of the variables of Structural Formula (I) is as follows:

R¹, R², Q, Y, J^{Y}, R^{a}, J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in any one of the first through thirteenth sets of values of the variables of Structural Formula (I).

Each of R⁴, R⁵, R⁶, and R⁸ independently is —H or C₁₋₆ alkyl optionally substituted with one or more substituents selected from the group consisting of

halogen, —CN, —OH, —NH₂, —NH(C₁₋₆alkyl), —N(C₁₋₆alkyl)₂, —O(C₁₋₆alkyl), and —O(C₁₋₆ haloalkyl); and R³ and R⁷, together with the atom(s) to which they are attached, form a 4-membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl). Examples of the heterocyclic ring formed with R³ and R⁷ include:

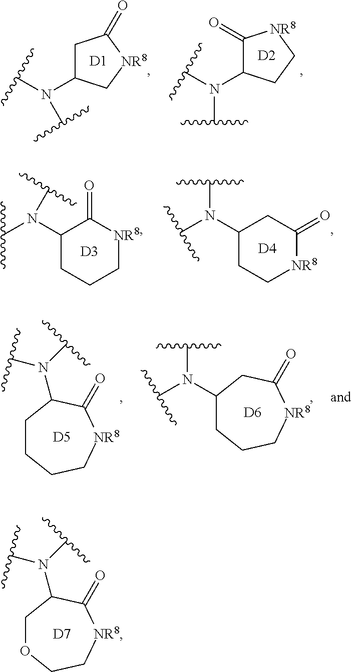

wherein each of rings D1-D7 is independently and optionally further substituted.

The remaining variables of Structural Formula (I) are each and independently as described above in the first set of values of the variables of Structural Formula (I).

A twentieth set of values of the variables of Structural Formula (I) is as follows:

R¹ is C₁₋₆ alkyl optionally substituted with one or more substituents selected from the group consisting of halogen, —CN, —OH, and —O(C₁-C₆ alkyl).

R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group.

Each of R³, R⁴, R⁵ and R⁶ independently is —H or an optionally substituted C₁₋₆ alkyl group; and each of R⁷ and R⁸ independently is —H, an optionally substituted C₁₋₆ aliphatic, optionally substituted C₃₋₈ carbocyclic; or optionally R³ and R⁷, together with the atoms to which they are attached, form an optionally substituted, 4-10 membered heterocyclic ring; or optionally R⁷ and R⁸, together with the nitrogen atom to which they are attached, form an optionally substituted, 4-10 membered heterocyclic ring.

Suitable substituents for the values of R², R³, R⁴, R⁵, R⁶, R⁷, and R⁸ are each and independently as described above in the first set of values of the variables of Structural Formula (I).

In a twenty first set of variables of Structural Formula (I), n is 0, and values of the remaining variables of Structural Formula (I) are each and independently as described above in any set of values of the variables of Structural Formula (I).

In another embodiment, the compounds of the invention are represented by Structural Formula (II):

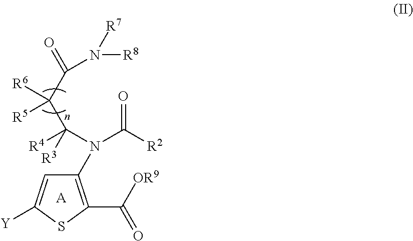

or pharmaceutically acceptable salts thereof. Values of the variables of Structural Formula (II) are each and independently as described above in any one of the first through twenty first sets of values of the variables of Structural Formula (I).

In a twenty second set of variables of Structural Formula (II), n is 0, and values of the remaining variables of Structural Formula (II) are each and independently as described above in any set of values of the variables of Structural Formula (I).

In a twenty third set of variables of Structural Formula (II), R³ and R⁷ does not optionally form a 4-10 membered heterocyclic ring optionally substituted with one or more instances of J^{E}, and values of the remaining variables of Structural Formula (II) are each and independently as described above in any set of values of the variables of Structural Formula (I).

In another embodiment, the compounds of the invention are represented by Structural Formulae (III) and (VIII):

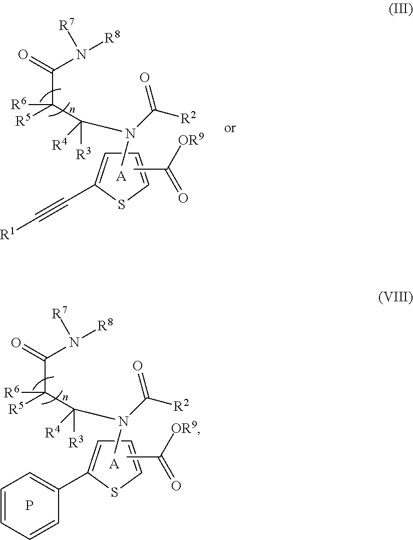

or pharmaceutically acceptable salts thereof. The first through twenty first sets of variables of Structural Formulae (III) and (VIII) are each and independently as described above in the first through eighteenth sets of values of the variables of Structural Formula (I), respectively, wherever applicable. Ring P is optionally substituted. Suitable substituents for ring P are as described above for J^{Y} in the first set of values of the variables of Structural Formula (I).

In a twenty second set of variables of Structural Formulae (III) and (VIII), n is 0, and values of the remaining variables of Structural Formulae (III) and (VIII) are each and independently as described above in any set of values of the variables of Structural Formula (I).

In a twenty third set of variables of Structural Formula (III), R³ and R⁷ does not optionally form a 4-10 membered heterocyclic ring optionally substituted with one or more instances of J^{E}, and values of the remaining variables of Structural Formula (III) are each and independently as described above in any set of variables of Structural Formula (I).

A twenty fourth set of values of the variables of Structural Formulae (III) and (VIII) is as follows:

R¹ is an optionally substituted C₁₋₆alkyl or optionally substituted C₃₋₈ carbocyclic group. Suitable substituents for the values of R¹ are each and independently as described above in the first set of values of the variables of Structural Formula (I). In one aspect, R¹ is C₁₋₆ alkyl or C₃₋₈ cycloalkyl, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl. In another aspect, R¹ is C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In yet another aspect, R¹ is C₁₋₆ alkyl optionally substituted with one or more substituents selected from the group consisting of halogen, —CN, —OH, and —O(C₁-C₆ alkyl). In yet another aspect, R¹ is t-butyl or isopropyl optionally substituted with one or more substituents selected from the group consisting of halogen, —CN, —OH, and —O(C₁-C₆ alkyl).

Each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —OC(O)OR^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Alternatively, each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Suitable substituents for the values of Q are each and independently as described above in the first set of values of the variables of Structural Formula (I).

The remaining variables of Structural Formula (I) are each and independently as described above in the first set of values of the variables of Structural Formula (I).

A twenty fifth set of values of the variables of Structural Formulae (III) and (VIII) is as follows:

R¹ and Q are each and independently as described above in the twenty fourth set of values of the variables of Structural Formulae (III) and (VIII).

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring. Suitable substituents for the values of R^{a} are each and independently as described above in the first set of values of the variables of Structural Formula (I).

The remaining variables of Structural Formula (I) are each and independently as described above in the first set of values of the variables of Structural Formula (I).

A twenty sixth set of values of the variables of Structural Formulae (III) and (VIII) is as follows:

R¹ and Q are each and independently as described above in the twenty fourth set of values of the variables of Structural Formulae (III) and (VIII).

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring. Suitable substituents for the values of R^{a} are each and independently as described above in the first set of values of the variables of Structural Formula (I).

Each of J^{1A}, J^{2A}, J^{7A}, and J^{9A} independently is halogen,

oxo, —CN, —OR^{a}, —NRR^{a}, —OC(O)R^{a}, —OC(O)OR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈ cyclo(haloalkyl), 5-6 membered optionally substituted heterocyclyl, or optionally substituted phenyl. Alternatively, Each of J^{1A}, J^{2A}, J^{7A}, and J^{9A} independently is halogen, oxo, —CN, —OR^{a}, —NRR^{a}, —OC(O)R^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), or phenyl.

Each of J^{1B}, J^{2B}, J^{7B}, and J^{9B} independently is halogen,

oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl.

Each of J^{1C}, J^{2C}, J^{7C}, and J^{9C} independently is halogen,

oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈cycloalkyl, C₃₋₈cyclo(haloalkyl), and phenyl.

The remaining variables of Structural Formula (I) are each and independently as described above in the first set of values of the variables of Structural Formula (I).

A twenty seventh set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group. In one aspect, R² is optionally substituted C₅-C₈ cycloalkyl or optionally substituted phenyl. Suitable substituents for the values of R² are each and independently as described above in the first set of values of the variables of Structural Formula (I).

The remaining variables of Structural Formula (I) are each and independently as described above in the first set of values of the variables of Structural Formula (I).

A twenty eighth set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is C₅-C₈ cycloalkyl optionally substituted with one or more substituents selected from the group consisting of halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In one aspect, R² is cyclohexyl optionally substituted with one or more substituents selected from the group consisting of halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In another aspect, R² is cyclohexyl optionally substituted with one or more instances of J^{2B} independently selected from the group consisting of halogen, —CN, —OH, —NH₂, —NH(C₁₋₆ alkyl), —N(C₁₋₆ alkyl)₂, —O(C₁₋₆alkyl), and C₁₋₆alkyl optionally substituted with one or more substituents selected from the group consisting of

halogen, —CN, —OH, —NH₂, —NH(C₁₋₆ alkyl), —N(C₁₋₆ alkyl)₂, —O(C₁₋₆alkyl), and —O(C₁₋₆ haloalkyl).

The remaining variables of Structural Formula (I) are each and independently as described above in the first set of values of the variables of Structural Formula (I).

A twenty ninth set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is cyclohexyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and C₁₋₆alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl).

Each of R³, R⁴, R⁵ and R⁶ independently is —H or an optionally substituted C₁₋₆ alkyl group; or optionally R³, together with R⁷ and the atom to which it is attached, forms an optionally substituted, 4-10 membered heterocyclic ring.

The remaining variables of Structural Formula (I) are each and independently as described above in the first set of values of the variables of Structural Formula (I).

A thirtieth set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R³, R⁴, R⁵ and R⁶ independently is —H or an optionally substituted C₁₋₆ alkyl group; and each of R⁷ and R⁸ independently is an optionally substituted C₁₋₆ aliphatic, optionally substituted C₃₋₈ carbocyclic, or optionally substituted, 4-8 membered heterocyclic group, or R⁷ and R⁸, together with the nitrogen atom to which they are attached, form an optionally substituted, 4-10 membered heterocyclic ring

The remaining variables of Structural Formula (I) are each and independently as described above in the first set of values of the variables of Structural Formula (I).

A thirty first set of variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R³, R⁴, R⁵ and R⁶ independently is —H or C₁₋₆ alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; and

each of R⁷ and R⁸ independently is —H; C₁₋₆ alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; or a C₃₋₈ carbocyclic or 4-8 membered heterocyclic group each of which optionally and independently substituted with one or more substituents selected from the group consisting of halogen,
oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl; or

R⁷ and R⁸, together with the atom to which they are attached, optionally form a 4-membered heterocyclic ring optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; or

for Structural Formula (III), R³ and R⁷, together with the atoms to which they are attached, optionally form a 4-10 membered heterocyclic ring optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl.

Examples of the heterocyclic ring formed with R³ and R⁷ are as described above.

The remaining variables of Structural Formula (I) are each and independently as described above in the first set of values of the variables of Structural Formula (I).

A thirty second set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R³, R⁴, R⁵ and R⁶ independently is —H or C₁₋₆alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.

Each of R⁷ and R⁸ independently is —H; C₁₋₆alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl; or a C₃₋₈ carbocyclic or 4-8 membered heterocyclic group each of which optionally and independently substituted with one or more substituents selected from the group consisting of halogen,
oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl.

The remaining variables of Structural Formula (I) are each and independently as described above in the first set of values of the variables of Structural Formula (I).

A thirty third set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R³, R⁴, R⁵ and R⁶ independently is —H or C₁₋₆alkyl optionally substituted with one or more substituents selected from the group consisting of

halogen, —CN, —OH, —NH₂, —NH(C₁₋₆alkyl), —N(C₁₋₆alkyl)₂, —O(C₁₋₆alkyl), and —O(C₁₋₆ haloalkyl).

R⁷, and R⁸ independently is —H or C₁₋₆alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆ haloalkyl); or R⁷ and R⁸, together with the atom to which they are attached, optionally form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl). The remaining variables of Structural Formula (I) are each and independently as described above in the first set of values of the variables of Structural Formula (I).

A thirty fourth set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R³, R⁴, R⁵ and R⁶ independently is as described above in the thirty third set of values of the variables of Structural Formulae (III) and (VIII).

R⁷ and R⁸ independently is —H or C₁₋₆ alkyl optionally substituted with one or more substituents selected from the group consisting of

halogen, —CN, —OH, —NH₂, —NH(C₁₋₆alkyl), —N(C₁₋₆ alkyl)₂, —O(C₁₋₆alkyl), and —O(C₁₋₆ haloalkyl); or R⁷ and R⁸, together with the atom to which they are attached, optionally form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl). The remaining variables of Structural Formula (I) are each and independently as described above in the first set of values of the variables of Structural Formula (I).

A thirty fifth set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R³, R⁴, R⁵ and R⁶ independently is as described above in the thirty third set of values of the variables of Structural Formulae (III) and (VIII).

R⁷ and R⁸, together with the atom to which they are attached, optionally form a 4-membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl). The heterocyclic ring formed with R⁷ and R⁸ can be a bridged or spiro ring. Examples of the heterocyclic ring formed with R³ and R⁷ are as described above.

The remaining variables of Structural Formula (I) are each and independently as described above in the first set of values of the variables of Structural Formula (I).

A thirty sixth set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R⁴, R⁵, R⁶, and R⁸ independently is —H or C₁₋₆ alkyl optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇cyclo(haloalkyl), and phenyl.

For Structural Formula (III), R³ and R⁷, together with the atom(s) to which they are attached, form a 4-10 membered heterocyclic ring optionally substituted with one or more substituents selected from the group consisting of halogen,

oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), and phenyl. In one aspect, R³ and R⁷, together with the atom(s) to which they are attached, form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl). Examples of the heterocyclic ring formed with R³ and R⁷ are as described above.

The remaining variables of Structural Formula (I) are each and independently as described above in the first set of values of the variables of Structural Formula (I).

A thirty seventh set of values of the variables of Structural Formulae (III) and (VIII) is as follows:

R¹ and Q are each and independently as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R² is as described above in the twenty ninth set of values of the variables of Structural Formulae (III) and (VIII).

Each of R⁴, R⁵, R⁶, and R⁸ independently is —H or C₁₋₆alkyl optionally substituted with one or more substituents selected from the group consisting of

halogen, —CN, —OH, —NH₂, —NH(C₁₋₆ alkyl), —N(C₁₋₆ alkyl)₂, —O(C₁₋₆alkyl), and —O(C₁₋₆ haloalkyl).

For Structural Formula (III), R³ and R⁷, together with the atom(s) to which they are attached, form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆ haloalkyl). Examples of the heterocyclic ring formed with R³ and R⁷ are as described above.

The remaining variables of Structural Formula (I) are each and independently as described above in the first set of values of the variables of Structural Formula (I).

A thirty eighth set of values of the variables of Structural Formulae (III) and (VIII) is as follows:

Q is as described above in the twenty fourth set of values of the variables of Structural Formulae (III) and (VIII).

R^{a} is as described above in the twenty fifth set of values of the variables of Structural Formulae (III) and (VIII).

J^{1A}, J^{2A}, J^{7A}, J^{9A}, J^{1B}, J^{2B}, J^{7B}, J^{9B}, J^{1C}, J^{2C}, J^{7C}, and J^{9C} are each and independently as described above in the twenty sixth set of values of the variables of Structural Formulae (III) and (VIII).

R¹ is C₁₋₆ alkyl optionally substituted with one or more substituents selected from the group consisting of halogen, —CN, —OH, and —O(C₁₋₆ alkyl).

R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group.

Each of R³, R⁴, R⁵ and R⁶ independently is —H or an optionally substituted C₁₋₆ alkyl group.

Each of R⁷ and R⁸ independently is —H, an optionally substituted C₁₋₆ aliphatic, optionally substituted C₃₋₈ carbocyclic; or, Structural Formula (III), optionally R³ and R⁷, together with the atoms to which they are attached, form an optionally substituted, 4-10 membered heterocyclic ring; or optionally R⁷ and R⁸, together with the nitrogen atom to which they are attached, form an optionally substituted, 4-10 membered heterocyclic ring. Substituents for the values of R², R³, R⁴, R⁵, R⁶, R⁷, and R⁸ are each and independently as described above in the first set of values of the variables of Structural Formula (I).

The remaining variables of Structural Formula (I) are each and independently as described above in the first set of values of the variables of Structural Formula (I).

In another embodiment, the compounds of the invention are represented by Structural Formulae (IV) and (IX):

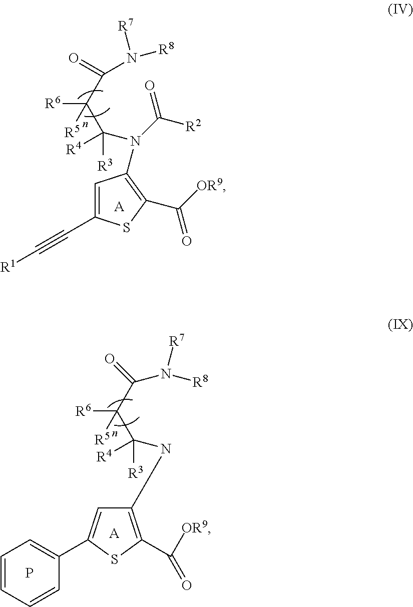

or pharmaceutically acceptable salts thereof. Values of the variables of Structural Formulae (IV) and (IX) are each and independently as described above in any one of the first through thirty eighth sets of values of the variables of Structural Formulae (III) and (VIII).

In another embodiment, the compounds of the invention are represented by Structural Formulae (V), (X), and (XI):

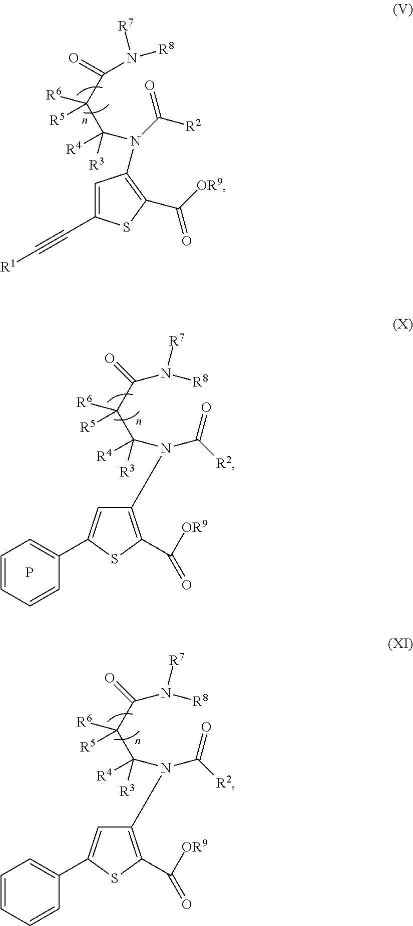

or pharmaceutically acceptable salts thereof. Values of the variables of Structural Formulae (V), (X), and (XI) are each and independently as described above in any one of the first through thirty eighth sets of values of the variables of Structural Formulae (III) and (VIII).

In another embodiment, the compounds of the invention are represented by Structural Formulae (VI) and (XII):

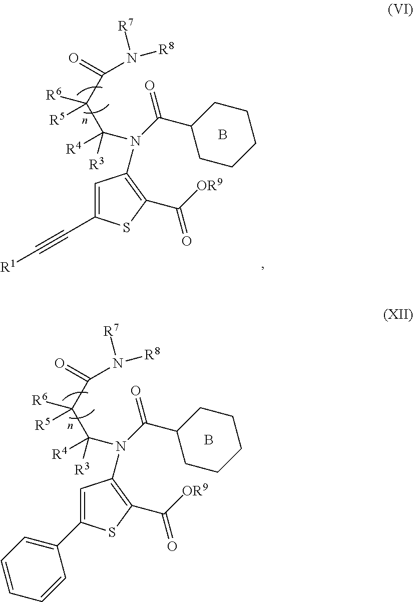

or pharmaceutically acceptable salts thereof, wherein ring B is optionally substituted with one or more instances of J^{2B}. Values of the variables of Structural Formulae (VI) and (XII) are each and independently as described above in any one of the first through thirty eighth sets of values of the variables of Structural Formulae (III) and (VIII).

In an additional set of value of the variables of Structural Formulae (VI) and (XII), J^{2B} is halogen, —CN, —OH, —NH₂, —NH(C₁₋₆ alkyl), —N(C₁₋₆ alkyl)₂, —O(C₁₋₆alkyl), or C₁₋₆alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆haloalkyl); and values of the remaining variables of Structural Formulae (VI) and (XII) are each and independently as described above in any one of the first through thirty eighth sets of values of the variables of Structural Formulae (III) and (VIII).

In yet an additional set of value of the variables of Structural Formulae (VI) and (XII), J^{2B} is C₁₋₆alkyl or —O(C₁₋₆alkyl); and values of the remaining variables of Structural Formulae (VI) and (XII) are each and independently as described above in any one of the first through thirty eighth sets of values of the variables of Structural Formulae (III) and (VIII).

In another embodiment, the compounds of the invention are represented by Structural Formulae (VII) and (XIII):

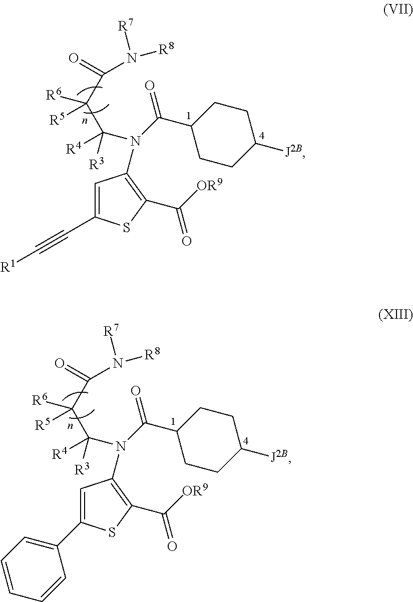

or pharmaceutically acceptable salts thereof. Values of the variables of Structural Formulae (VII) and (XIII) are each and independently as described above in any one of the first through thirty eighth sets of values of the variables of Structural Formulae (III) and (VIII).

In an additional set of value of the variables of Structural Formulae (VII) and (XIII), J^{2B} is halogen, —CN, —OH, —NH₂, —NH(C₁₋₆ alkyl), —N(C₁₋₆alkyl)₂, —O(C₁₋₆alkyl), or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆haloalkyl),; and values of the remaining variables of Structural Formulae (VII) and (XIII) are each and independently as described above in any one of the first through thirty eighth sets of values of the variables of Structural Formulae (III) and (VIII).

In yet an additional set of value of the variables of Structural Formulae (VII) and (XIII), J^{2B} is C₁₋₆alkyl or —O(C₁₋₆alkyl); and values of the remaining variables of Structural Formula (VI) are each and independently as described above in any one of the first through thirty eighth sets of values of the variables of Structural Formulae (III) and (VIII).

In yet an additional set of value of the variables of Structural Formulae (VII) and (XIII), J^{2B} is C₁₋₆alkyl, such as methyl; and values of the remaining variables of Structural Formulae (VII) and (XIII) are each and independently as described above in any one of the first through thirty eighth sets of values of the variables of Structural Formulae (III) and (VIII).

In yet an additional set of value of the variables of Structural Formulae (VII) and (XIII), J^{2B} is C₁₋₆alkyl or —O(C₁₋₆alkyl); R¹ is C₁₋₆ alkyl, such as t-butyl or isopropyl; and values of the remaining variables of Structural Formulae (VII) and (XIII) are each and independently as described above in any one of the first through thirty eighth sets of values of the variables of Structural Formulae (III) and (VIII).

In yet an additional set of value of the variables of Structural Formulae (VII) and (XIII),

J^{2B} is C₁₋₆alkyl; R¹ is C₁₋₆ alkyl, such as t-butyl or isopropyl;

each of R³, R⁴, R⁵ and R⁶ independently is —H or C₁₋₆ alkyl optionally substituted with one or more substituents selected from the group consisting of

halogen, —CN, —OH, —NH₂, —NH(C₁₋₆alkyl), —N(C₁₋₆ alkyl)₂, —O(C₁₋₆alkyl), and —O(C₁₋₆ haloalkyl); and values of the remaining variables of Structural Formulae (VII) and (XIII) are each and independently as described above in any one of the first through thirty eighth sets of values of the variables of Structural Formulae (III) and (VIII).

In yet an additional set of value of the variables of Structural Formulae (VII) and (XIII),

J^{2B} is C₁₋₆ alkyl; R¹ is C₁₋₆ alkyl, such as t-butyl or isopropyl;

each of R³, R⁴, R⁵ and R⁶ independently is —H or C₁₋₆ alkyl optionally substituted with one or more of —O(C₁₋₆haloalkyl); and

values of the remaining variables of Structural Formulae (VII) and (XIII) are each and independently as described above in any one of the first through thirty eighth sets of values of the variables of Structural Formulae (III) and (VIII).

In yet an additional set of value of the variables of Structural Formulae (VII) and (XIII),

J^{2B} is C₁₋₆ alkyl; R¹ is C₁₋₆alkyl, such as t-butyl or isopropyl;

each of R³, R⁴, R⁵ and R⁶ independently

is —H, —CH₃, —CH₂CH₃, —CH₂CH₂CH₃, —CH₂—OCH₃, —CH₂CH₂—OCH₃, —CH₂CH₂—OCH₂CH₃, or —CH₂CH₂—OCH₂CH₃; and

values of the remaining variables of Structural Formulae (VII) and (XIII) are each and independently as described above in any one of the first through thirty eighth sets of values of the variables of Structural Formulae (III) and (VIII).

In some embodiments, J^{2B} is trans to the carbonyl group at position 1 of the cyclohexyl ring to which J^{2B} is attached.

In some embodiments, the compounds of the invention are represented by any one of Structural Formulae (I)-(XIII), wherein:

R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —OC(O)O(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), phenyl, and 5-6 membered heterocycle optionally substituted with one or more substituents selected from the group consisting of oxo and C₁₋₆ alkyl; and

values of the remaining variables are each and independently as described above.

In some embodiments, the compounds of the invention are represented by any one of Structural Formulae (I)-(XIII), wherein:

R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; and

values of the remaining variables are each and independently as described above.

In some embodiments, the compounds of the invention are represented by any one of Structural Formulae (I)-(XIII), wherein:

R⁹ is —H or C₁₋₆alkyl optionally substituted with —OC(O)(C₁-C₆ alkyl); and

values of the remaining variables are each and independently as described above.

In some embodiments, the compounds of the invention are represented by any one of Structural Formulae (I)-(XIII), wherein:

R⁹ is —H or C₁₋₆ alkyl optionally substituted with —OC(O)O(C₁-C₆ alkyl); and

values of the remaining variables are each and independently as described above.

In some embodiments, the compounds of the invention are represented by any one of Structural Formulae (I)-(XIII), wherein:

R⁹ is —H or C₁₋₆ alkyl optionally substituted with 5-6 membered heterocycle optionally substituted with one or more substituents selected from the group consisting of oxo and C₁₋₆ alkyl; and

values of the remaining variables are each and independently as described above.

In some embodiments, the compounds of the invention are represented by any one of Structural Formulae (I)-(XIII) or pharmaceutically acceptable salts thereof, wherein:

R⁹ is —H or C₁₋₆alkyl; and

values of the remaining variables are each and independently as described above.

In some embodiments, the compounds of the invention are represented by any one of Structural Formulae (I)-(XIII) or pharmaceutically acceptable salts thereof, wherein:

R⁹ is —H; and

values of the remaining variables are each and independently as described above.

In some embodiments, the compounds of the invention are represented by Structural Formula (XX) or (XXI), or pharmaceutically acceptable salts thereof:

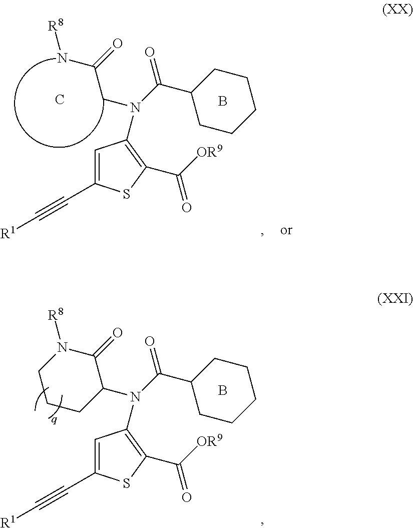

or a pharmaceutically acceptable salt thereof, wherein the values of:

R¹ is C₁₋₆alkyl optionally substituted with one or more substituents selected from the group consisting of halogen, —CN, —OH, and —O(C₁-C₆ alkyl);

R⁸ is —H or C₁₋₄ alkyl optionally substituted with one or substituents selected from the group consisting of halogen, hydroxyl, —O(C₁₋₄alkyl), —NH2, —NH(C₁₋₄ alkyl), and —N(C₁₋₄ alkyl)₂;

R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —OC(O)O(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), phenyl, and 5-6 membered heterocycle optionally substituted with one or more substituents selected from the group consisting of oxo and C₁₋₆ alkyl;

ring B is optionally substituted with one or more instances of J^{2B}; ring C is a 5-7 membered heterocycle optionally substituted with one or substituents selected from the group consisting of halogen, hydroxyl, —O(C₁₋₄alkyl), —NH2, —NH(C₁₋₄ alkyl), and —N(C₁₋₄alkyl)₂; and

q is 0, 1 or 2.

In some embodiments, the compounds of the invention are represented by Structural Formula (XX) or (XXI), or pharmaceutically acceptable salts thereof, wherein:

R¹ is t-butyl or isopropyl;

R⁸ is —H or C₁₋₄ alkyl optionally substituted with one or substituents selected from the group consisting of halogen, hydroxyl, —O(CH₃), —O(C₂H₅), —NH₂, —NH(CH₃), and —N(CH₃)₂;

J^{2B} is halogen, —CN, —OH, —NH₂, —NH(C₁₋₆ alkyl), —N(C₁₋₆alkyl)₂, —O(C₁₋₆alkyl), or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆haloalkyl); and

The other values are as described above for Structural Formulae (XX) and (XXI).

In some embodiments, the compounds of the invention are represented by Structural Formula (XX) or (XXI), or pharmaceutically acceptable salts thereof, wherein wherein R⁸ is —H or C₁₋₄alkyl.

In some embodiments, the compounds of the invention are represented by Structural Formula (XX) or (XXI), or pharmaceutically acceptable salts thereof, wherein R⁹ is —H.

In some embodiments, the compounds of the invention are represented by Structural Formula (XX) or (XXI), or pharmaceutically acceptable salts thereof, wherein J^{2B} is —CH₃ or —O(CH₃).

In some embodiments, the compounds of the invention are represented by Structural Formula (XX) or (XXI), or pharmaceutically acceptable salts thereof, wherein the values of the variables of Structural Formulae (XX) and (XXI) are each and independently as described above for any one of Structural Formulae (I)-(XIII).

In some embodiments, the compounds of the invention are pharmaceutically acceptable salts represented by any one of Structural Formulae (I)-(XIII), wherein values of the remaining variables are each and independently as described above.

In some embodiments, the compounds of the invention are pharmaceutically acceptable salts represented by any one of Structural Formulae (XX) and (XXI), wherein values of the remaining variables are each and independently as described above.

In some embodiments, the compounds of the invention are represented by any one of Structural Formulae (I)-(XIII) or pharmaceutically acceptable salts thereof, wherein:

n is 0; and

values of the remaining variables are each and independently as described above.

In some embodiments, the compounds of the invention are represented by Structural Formula (XX) or (XXI), or pharmaceutically acceptable salts thereof, wherein R⁹ is —H or C₁₋₆alkyl optionally substituted with —OC(O)(C₁-C₆ alkyl), —OC(O)O(C₁-C₆ alkyl), or 5-6 membered heterocycle optionally substituted with one or more substitutents selected from oxo and C₁₋₆alkyl.

In some embodiments, the compounds of the invention are represented by any one of Structural Formulae (I)-(V) or pharmaceutically acceptable salts thereof, wherein:

R² is optionally substituted C₅-C₈ cycloalkenyl. Suitable substitutents are as described above for Structural Formula (I).

Values of the other variables are as described above in any one of the embodiments for Structural Formulae (I)-(V).

In some embodiments, the compounds of the invention are represented by any one of the structural formulae depicted in FIG. 1, or pharmaceutically acceptable salts thereof. In other embodiments, the compounds of the invention are represented by any one of the structural formulae depicted in FIG. 2 or pharmaceutically acceptable salts thereof. In yet other embodiments, the compounds of the invention are represented by any one of the structural formulae depicted in FIG. 3 or pharmaceutically acceptable salts thereof. In yet other embodiments, the compounds of the invention are represented by any one of Compounds 1-120, 121-154, 156-173, and 174-191, or pharmaceutically acceptable salts thereof.

As used herein, a reference to compound(s) of the invention, for example compound(s) of Structural Formula (I), or compound(s) of claim 1, will include pharmaceutically acceptable salts thereof.

The compounds according to the invention described herein can be prepared by any suitable method known in the art. For example, the compounds can be prepared in accordance with procedures described in U.S. Pat. No. 6,881,741, US 2005/0009804, US 2006/0276533, WO 2002/100851, and WO 08/58393, the disclosures of which are hereby incorporated by reference.

The compounds of the invention (e.g., compounds of Structural Formulae (I)-(VII) can be prepared as depicted in General Schemes 1-7. For example, the compounds of Structural Formulae (I)-(V) can be prepared as shown in General Schemes 1-5, respectively; the compounds of Structural Formula (VI) as shown in General Schemes 6A and 6B; and the compounds of Structural Formula (VII) as shown in General Schemes 7A and 7B. The compounds of Structural Formulae (VIII)-(XIII) can be prepared as shown in General Scheme 1, and as shown in General Schemes 8-13. The compounds of Structural Formulae (XX) can be prepared as shown in General Scheme 1, and as shown in General Schemes 20A-20B. Compound depicted in these schemes (e.g., Compounds (1a)-(1p), (2a)-(2p), (3a)-(3p), (4a)-(4p), (5a)-(5p), (6a)-(6p), (7a)-(7p), (8a)-(8p), (9a)-(9p), (10a)-(10p), (11a)-(11p), (12a)-(12p), (13a)-(13p), and (20a)-(20p)) can be prepared by any suitable method known in the art. Any suitable condition(s) known in the art can be employed in the invention for each step depicted in the schemes.

In one embodiment, the present invention provides methods of preparing a compound represented by Structural Formula (I). In a specific embodiment, as shown in General Scheme 1, the methods comprise the step of reacting compound (1g) with compound (1h), (R⁸R⁷N)C(O)(CR⁵R⁶)ₙC(R³R⁴)—X (wherein X is a suitable leaving group as described above, such as F, Cl, Br, I, OMs, OTs, OTf, ONs, OBs, OR, OC(O)R, OC(═NR)NR, OBt, OAt, etc. (e.g. Han, et al., Tetrahedron 2004, 2447) to form compound (1i), a compound of Structural Formula (I) where R⁹ is -Me. In another specific embodiment, as shown in General Scheme 1, the methods comprise the step of reacting compound (1p) with compound (1f), X—C(O)R² (wherein X is a suitable leaving group as described above) to form compound (1i), a compound of Structural Formula (I) where R⁹ is -Me. Compound (1i), if desired, can then optionally further be hydrolyzed to from a compound of Structural Formula (I) where R⁹ is —H, i.e., the —C(O)OMe group at ring A being hydrolyzed to form —C(O)OH under a suitable condition(s) (e.g., a basic condition such as the presence of LiOH). Optionally, if desired, the —C(O)OH group can further be reacted with a suitable reagent(s) known in the art to form compounds of Structural Formula (I) having other than —H for R⁹. Any suitable condition known in the art can be employed for each step described in General Scheme 1. Specific exemplary conditions are described in the scheme. Exemplary detailed procedures are described below in the Exemplification section.

Compounds (1g) and (1p) can be prepared by any suitable method known in the art. In some specific embodiments, the methods further include the step of preparing compound (1g) or (1p), as shown in General Scheme 1. For example, compound (1g) can be prepared by reacting compound (1e) with compound (1f), X—C(O)R² (wherein X is a suitable leaving group as described above). Treatment of compound (1a) with trifluoroacetic anhydride can produce compound (1b); iodine can then be introduced into ring A by reacting compound (1b) with I₂ under a suitable condition(s); treatment of compound (1c) with a base, such as K₂CO₃, can then produce compound (1d); reaction of compound (1d) with Y—H for Y═(C₂ aliphatic group)-R¹, or with YB(OR^{k})₂ (wherein R^{k} is —H, C₁₋₆ alkyl, benzyl, etc.) for Y=aryl, heteroaryl, carbocycle, or heterocycle under suitable coupling condition(s) (for example, as indicated in General Scheme 1) can produce compound (1e). For example, compound (1p) can be prepared by reacting compound (1n) with compound (1o), (R⁸R⁷N)C(O)[(C(R⁵R⁶)]ₙC(R³R⁴)—NH₂. Compound (1n) can be prepared by reacting either compound (1l) or compound (1m) with Y—H for Y═(C₂ aliphatic group)-R¹, or with YB(OR^{k})₂ (wherein R^{k} is —H, C₁₋₆ alkyl, benzyl, etc.) for Y=aryl, heteroaryl, carbocycle, or heterocycle under suitable coupling condition(s) (for example, as indicated in General Scheme 1). Compound (1l) can be prepared by reacting compounds (1j) with I₂ under a suitable condition(s) (for example, as indicated in General Scheme 1). Compound (1m) can be prepared by carboxylating compound (1k) under a suitable condition(s) (for example, as indicated in General Scheme 1).

In another embodiment, the methods are as described in General Scheme 2. General Scheme 2 shows a general synthetic scheme for the compounds of Structural Formula (II). The synthetic details are each and independently as described above for General Scheme 1. For example, compounds (2a)-(2p) are each independently as described in General Scheme 1 for compounds (1a)-(1p).

In yet another embodiment, the methods are as described in General Scheme 3. General Scheme 3 shows a general synthetic scheme for the compounds of Structural Formula (III). The synthetic details are each and independently as described above for General Scheme 1. For example, compounds (3a)-(3p) are each independently as described in General Scheme 1 for compounds (1a)-(1p).

In yet another embodiment, the methods are as described in General Scheme 4. General Scheme 4 shows a general synthetic scheme for the compounds of Structural Formula (IV). The synthetic details are each and independently as described above for General Scheme 1. For example, compounds (4a)-(4p) are each independently as described in General Scheme 1 for compounds (1a)-(1p).

In yet another embodiment, the methods are as described in General Scheme 5. General Scheme 5 shows a general synthetic scheme for the compounds of Structural Formula (V). The synthetic details are each and independently as described above for General Scheme 1. For example, compounds (5a)-(5p) are each independently as described in General Scheme 1 for compounds (1a)-(1p).

In yet another embodiment, the methods are as described in General Scheme 6A or 6B. General Schemes 6A and 6B show general synthetic schemes for the compounds of Structural Formula (VI). The synthetic details are each and independently as described above for General Scheme 1. Compounds (6a)-(6p) are each independently as described in General Scheme 1 for compounds (1a)-(1p), respectively.

In yet another embodiment, the methods are as described in General Scheme 7A or 7B. General Schemes 7A and 7B show general synthetic schemes for the compounds of Structural Formula (VII). The synthetic details are each and independently as described above for General Scheme 1. Compounds (7a)-(7p) are each independently as described in General Scheme 1 for compounds (1a)-(1p), respectively.

In yet other embodiments, the methods are as described in General Schemes 8-13. General Schemes 8-13 show general synthetic schemes for the compounds of Structural Formulae (VIII)-(XIII), respectively. The synthetic details are each and independently as described above for General Scheme 1. Compounds (8a)-(8p), (9a)-(9p), (10a)-(10p), (11a)-(11p), (12a)-(12p), and (13a)-(13p) are each independently as described in General Scheme 1 for compounds (1a)-(1p), respectively.

In yet another embodiment, the methods are as described in General Schemes and 21. General Schemes 20 and 21 show general synthetic schemes for the compounds of Structural Formula (XX) — (XXI), respectively. The synthetic details are each and independently as described above for General Scheme 1. Compounds depicted in the schemes can be prepared by any suitable method known in the art. For example, reaction between Compounds (20n) and (20o) can generate Compound (20a), and subsequent reaction with Compound (20f) can produce Compound (20i). Compound (20i), if desired, can then optionally further be hydrolyzed to from a compound of Structural Formula (XX) where R⁹ is —H, i.e., the —C(O)OMe group is hydrolyzed to form —C(O)OH under a suitable condition(s) (e.g., a basic condition such as the presence of LiOH). Optionally, if desired, the —C(O)OH group can further be reacted with a suitable reagent(s) known in the art to form compounds of Structural Formula (XX) having other than —H for R⁹. Any suitable condition known in the art can be employed for each step described in General Scheme 20. The synthetic details for each step depicted in General Scheme 21 are each and independently as described above for General Scheme 20. Specific exemplary conditions are described in the schemes. Exemplary detailed procedures are described below in the Exemplification section.

Compounds (20j), (20k), (201), (20m), (20n), (20o), (20f), (21j), (21k), (21l), (21m), (21n), (21o), and (21f) can be prepared by any suitable method known in the art. For example, compound (20o) can be prepared as depicted in General Scheme 23, for example, from either Compound (23a) or (23e), as shown in the scheme. Similarly, compound (21o) can be prepared as depicted in General Scheme 22, for example, from either Compound (22a) or (22e). Any suitable condition known in the art can be employed for each step described in General Schemes 22 and 23. Specific exemplary conditions are described in the schemes. Exemplary detailed procedures are described below in the Exemplification section.

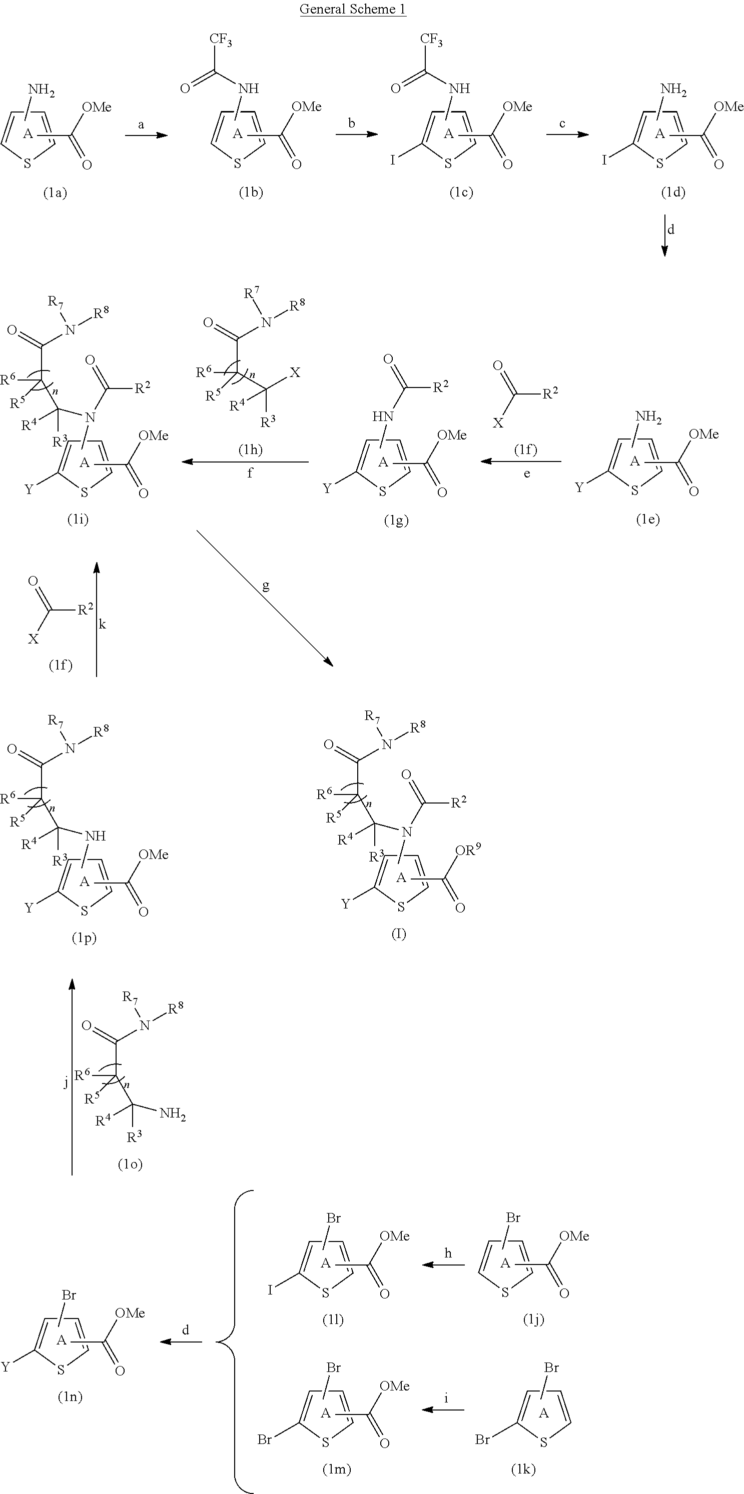

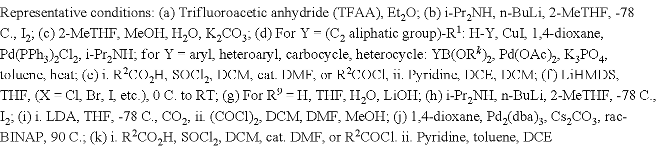

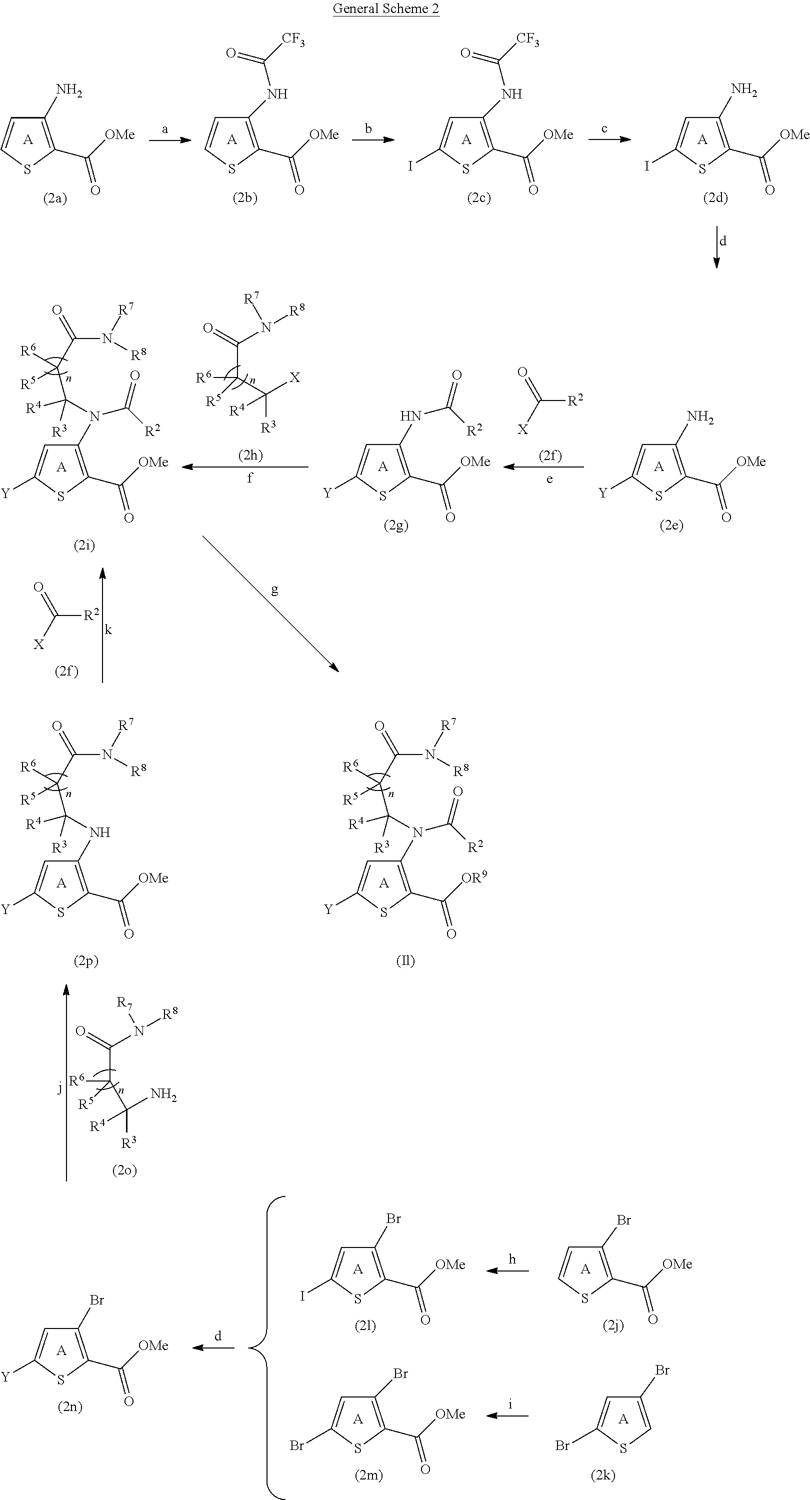

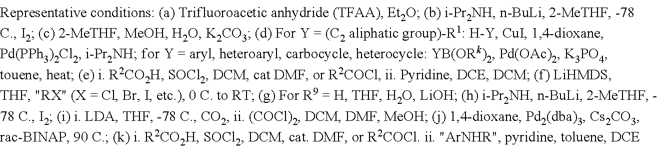

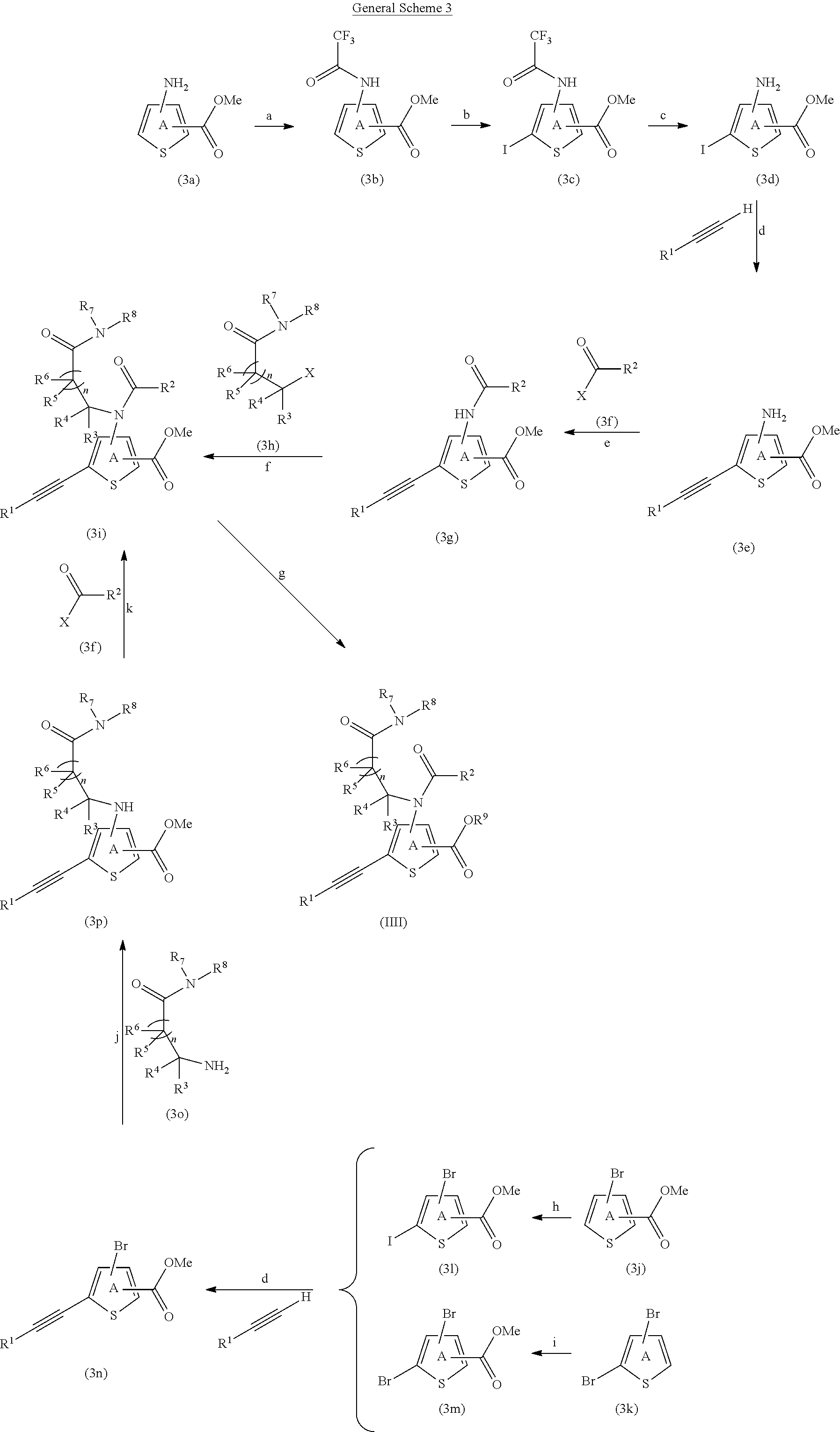

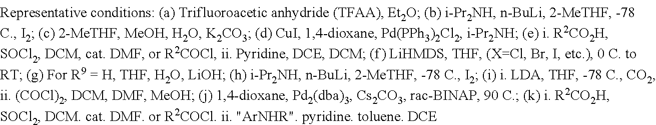

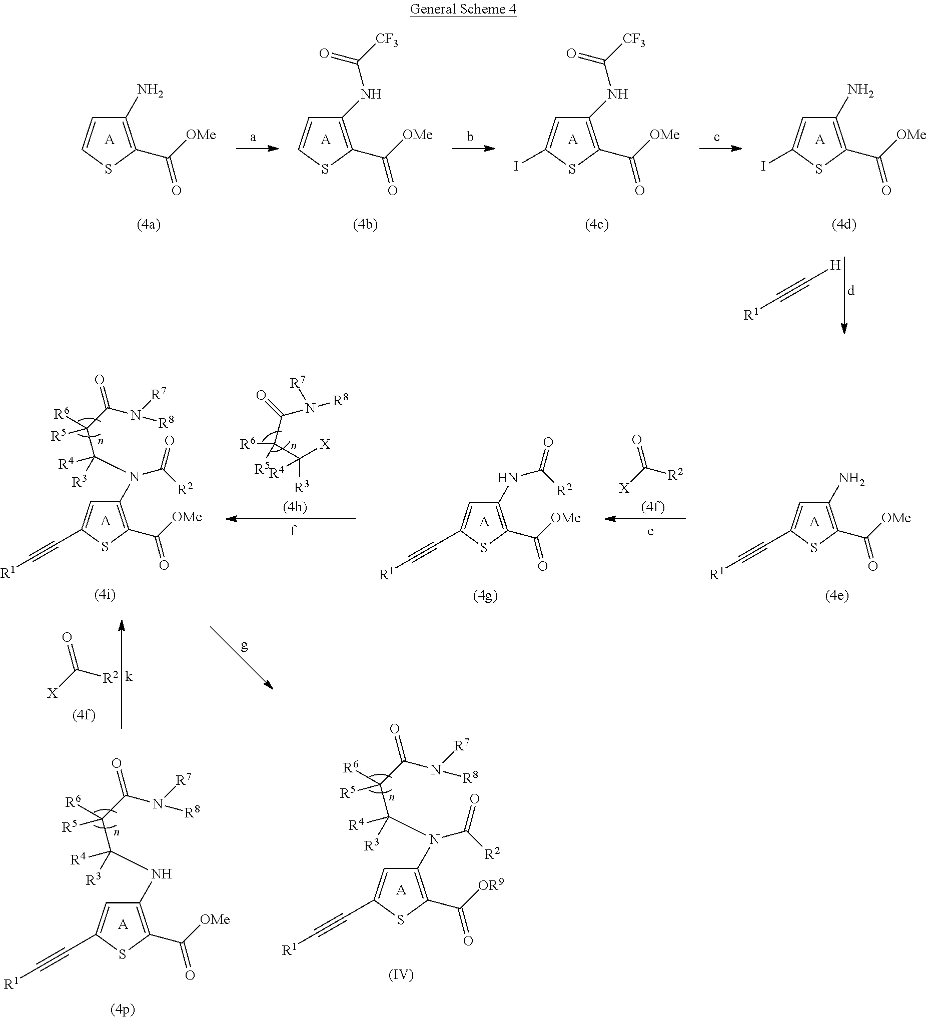

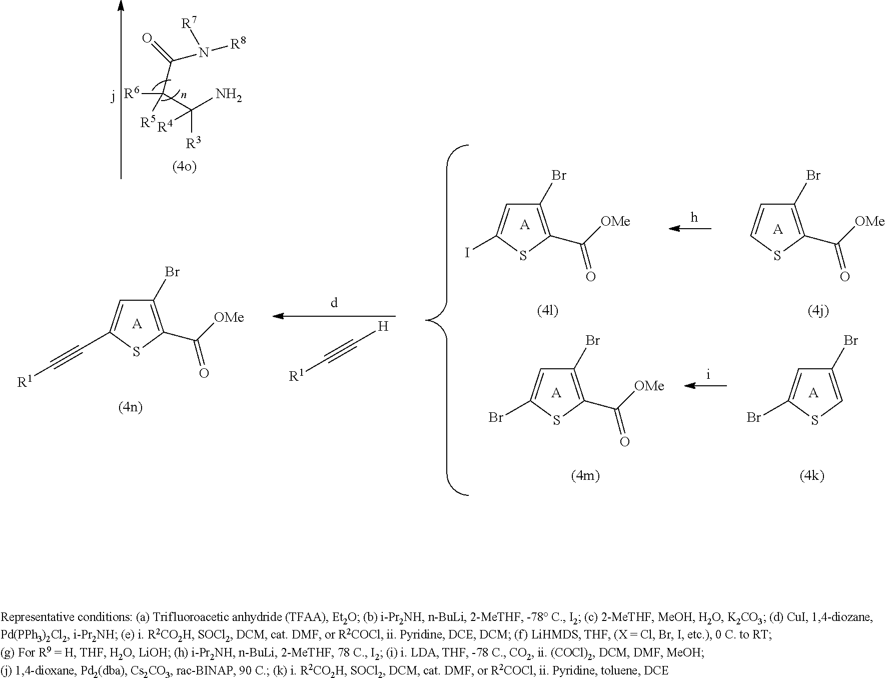

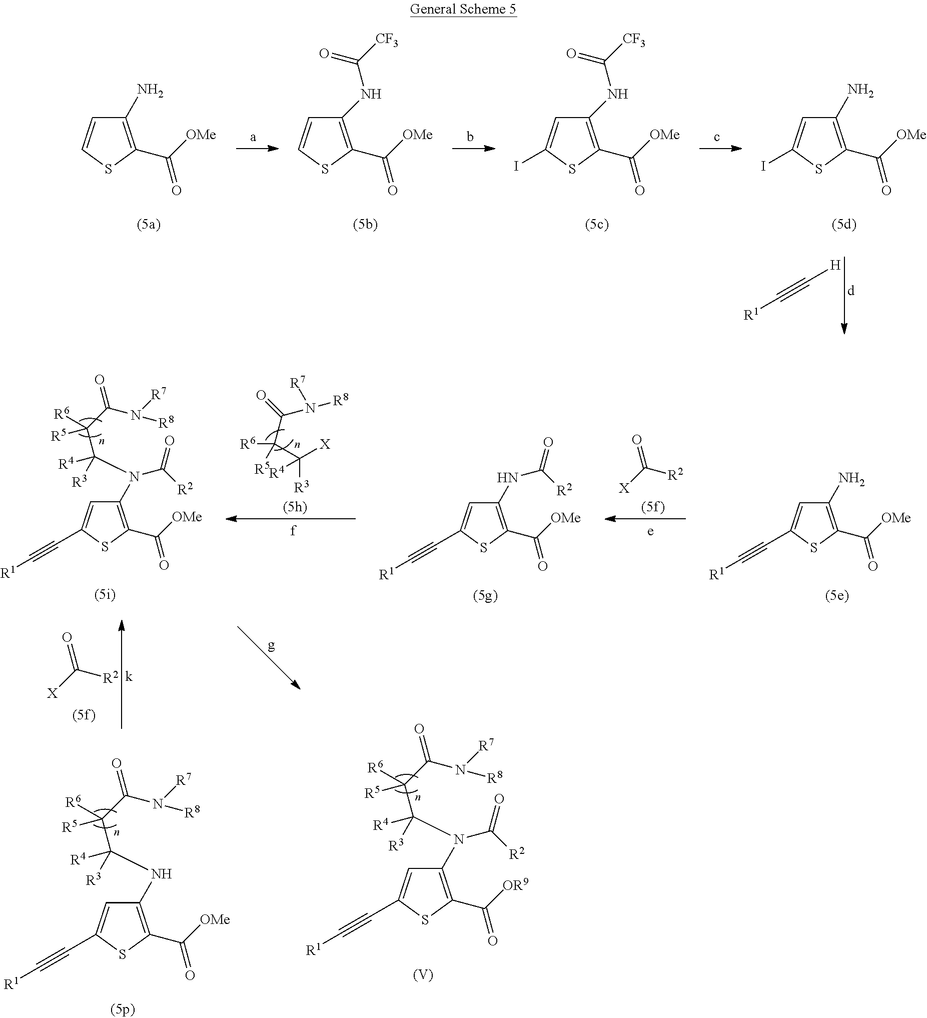

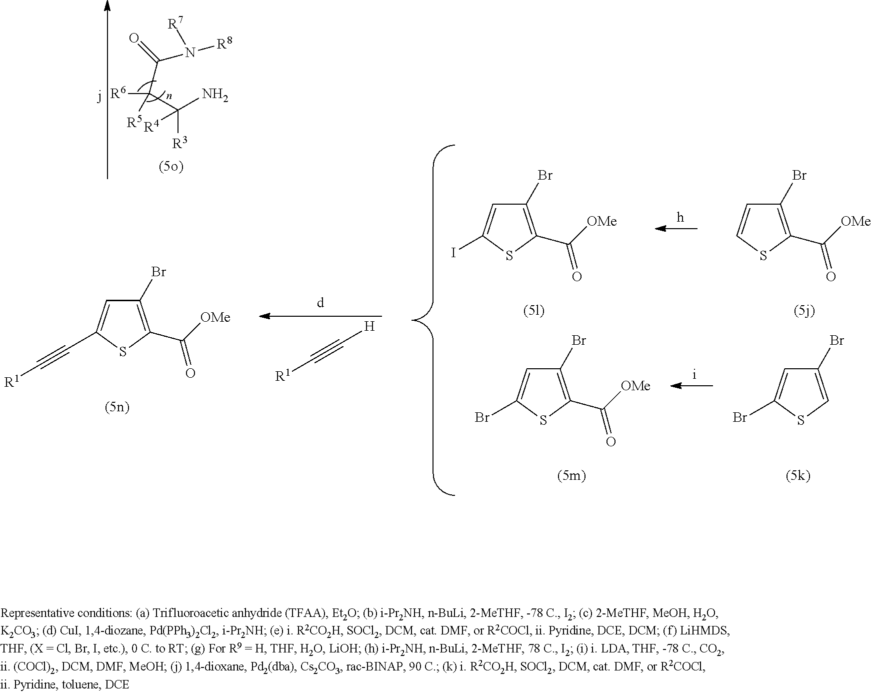

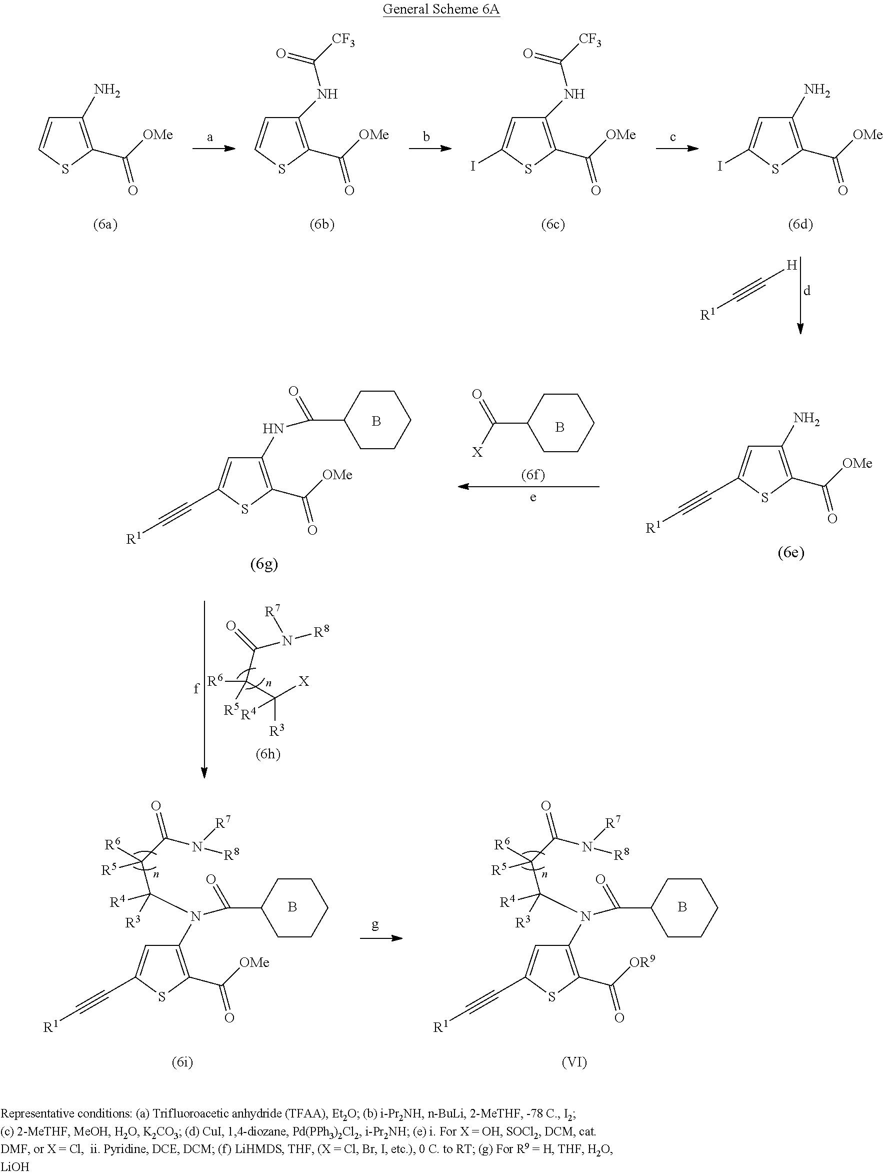

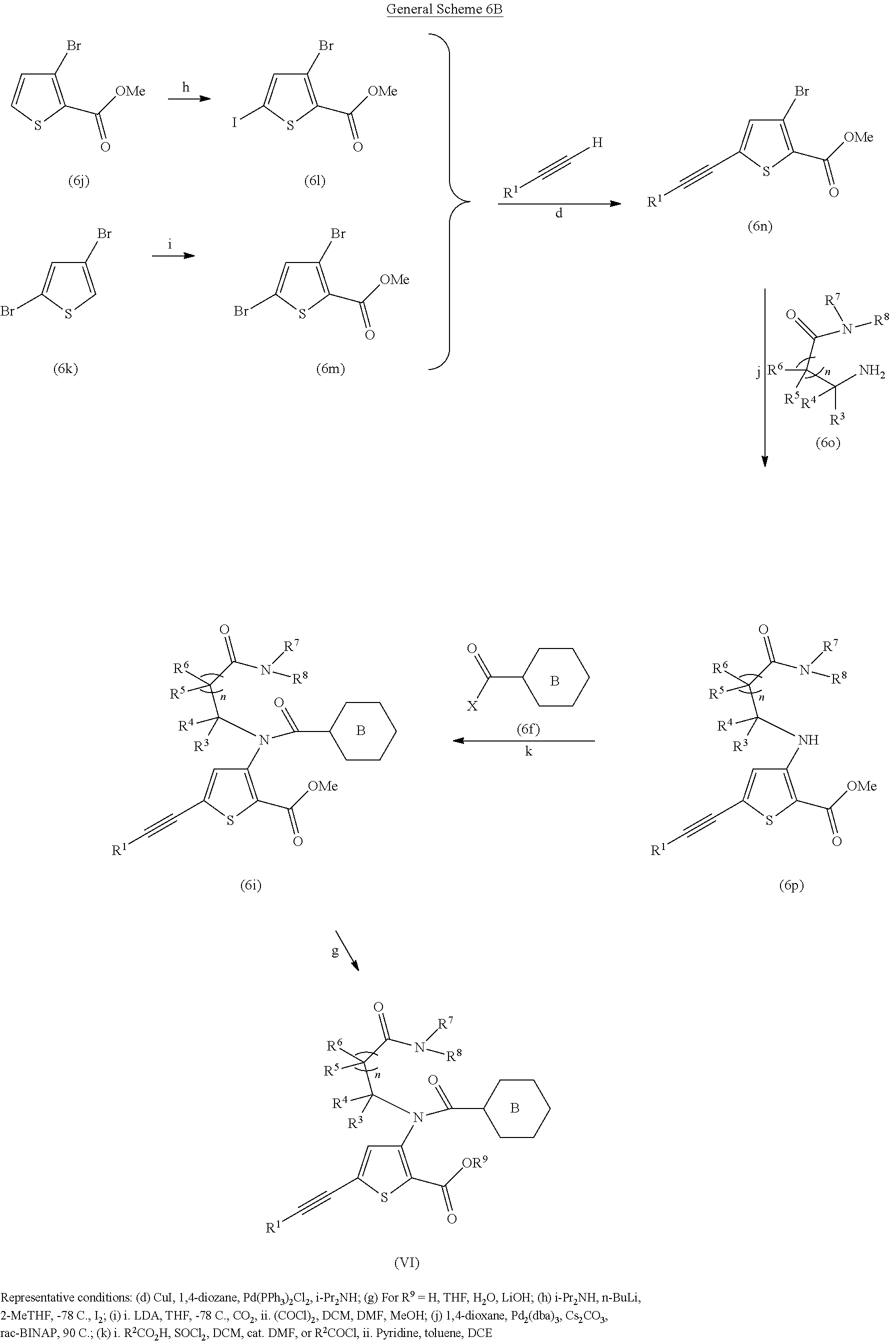

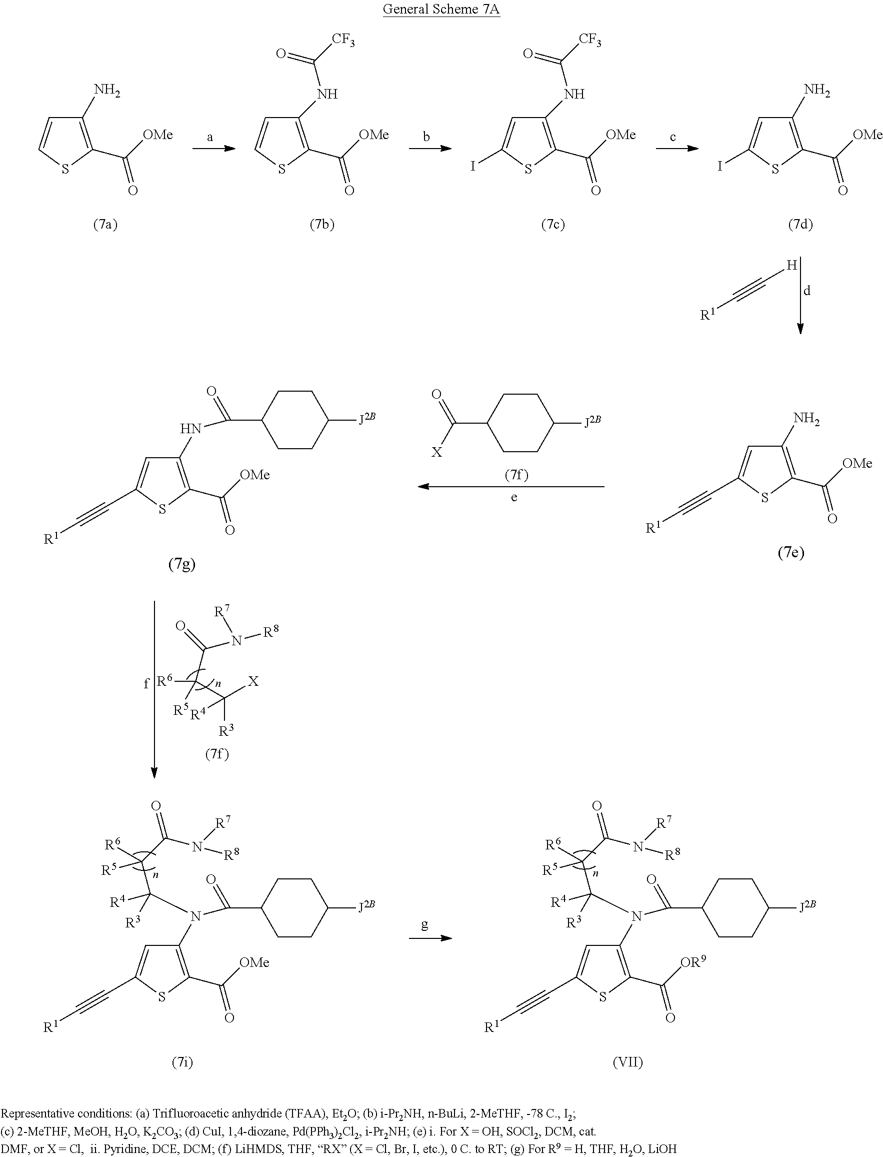

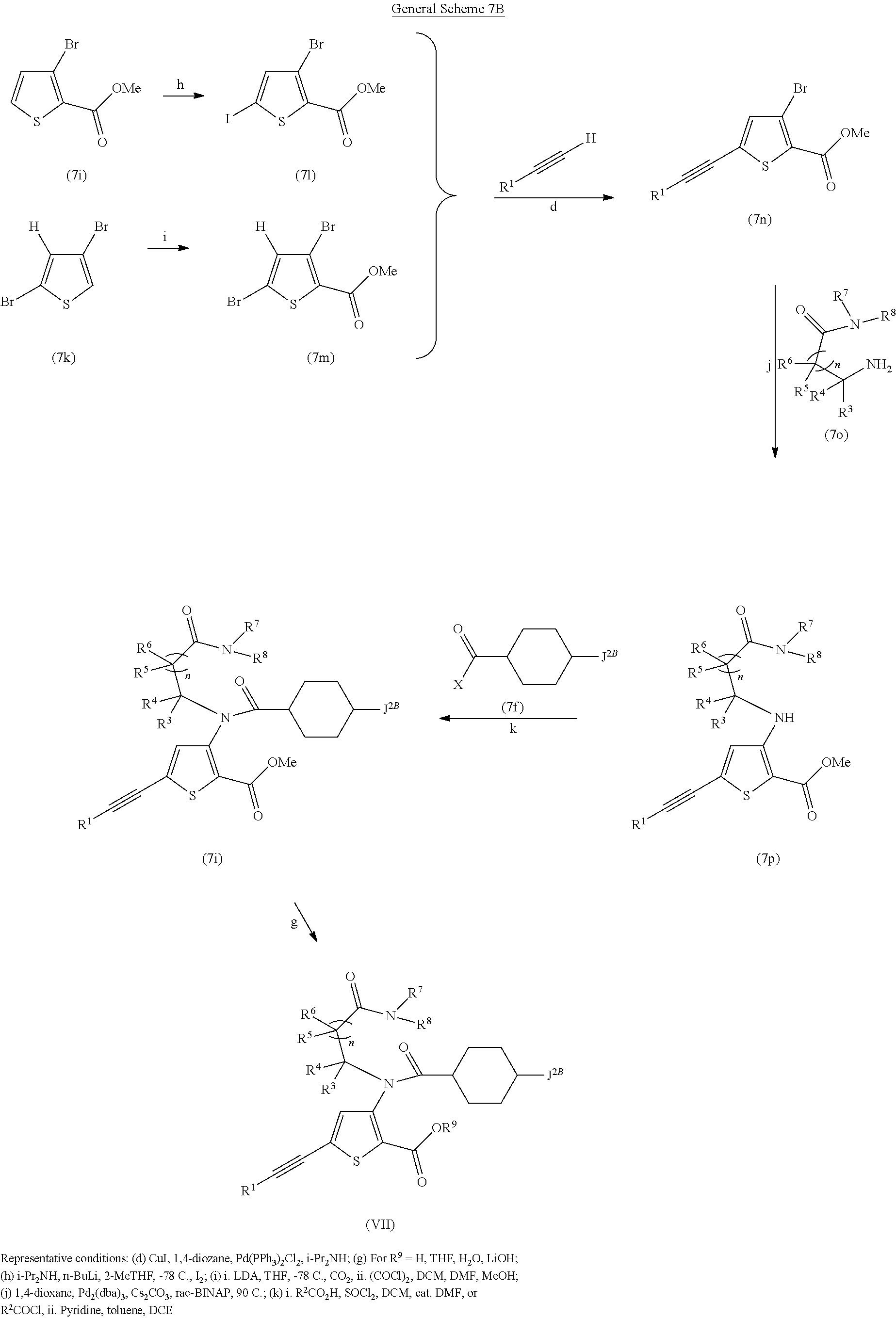

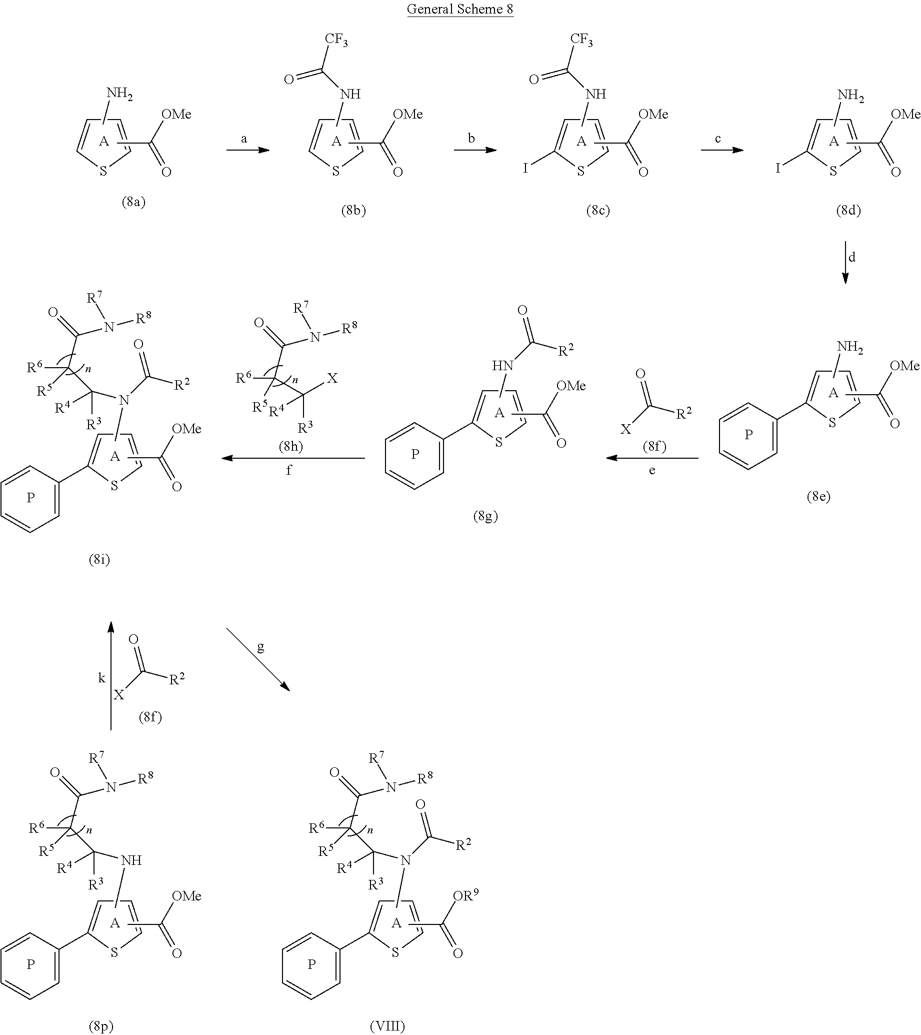

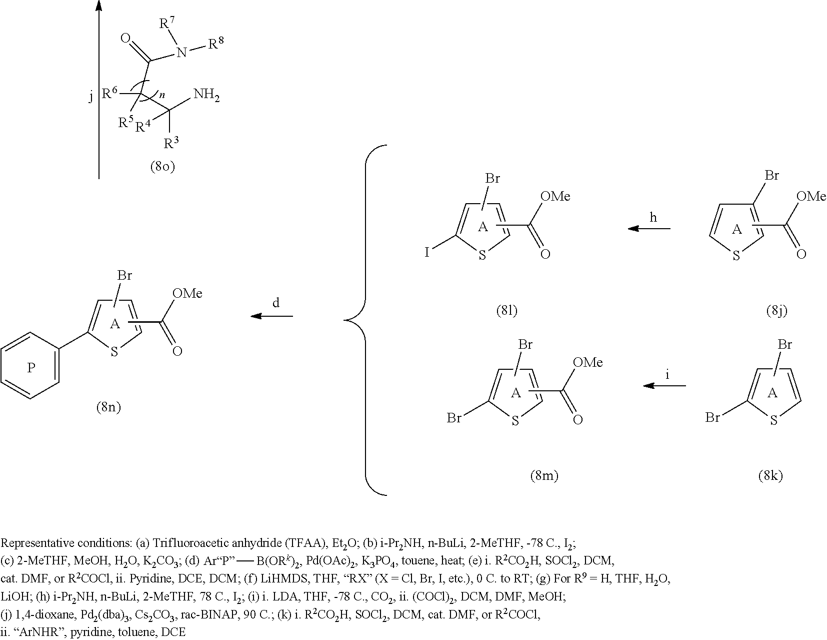

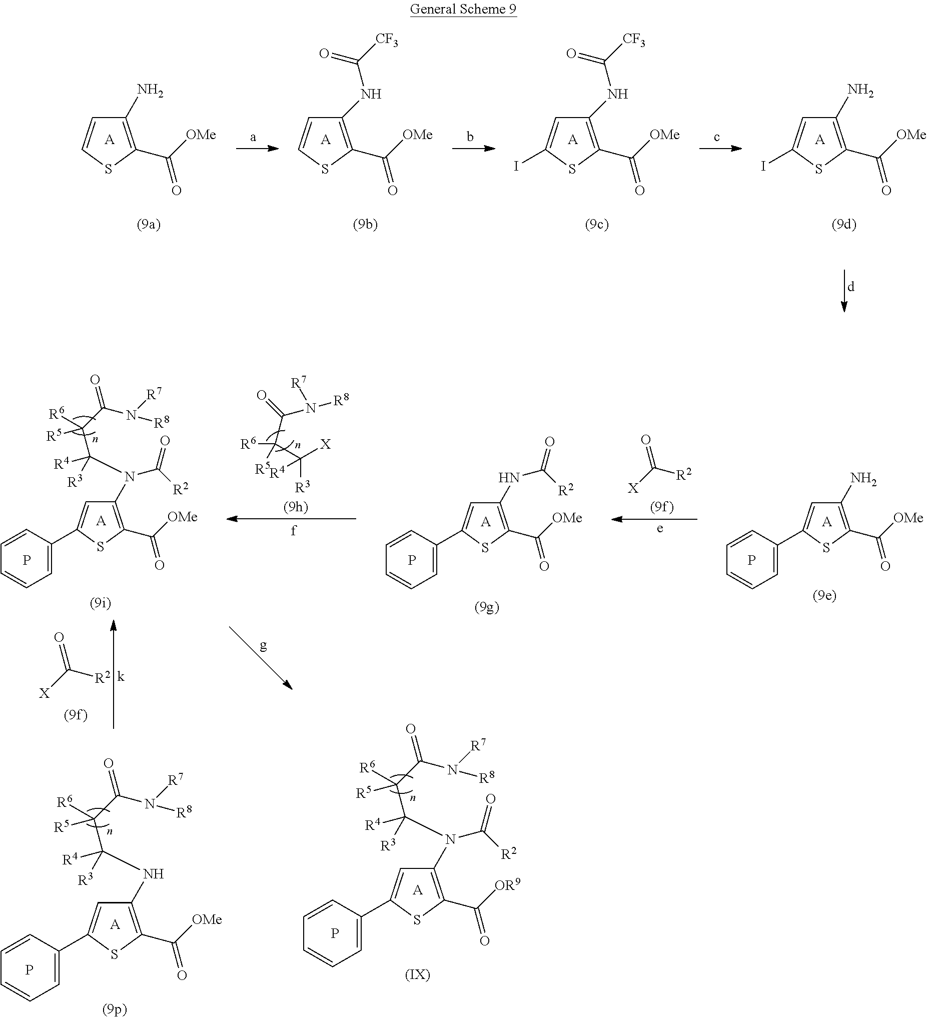

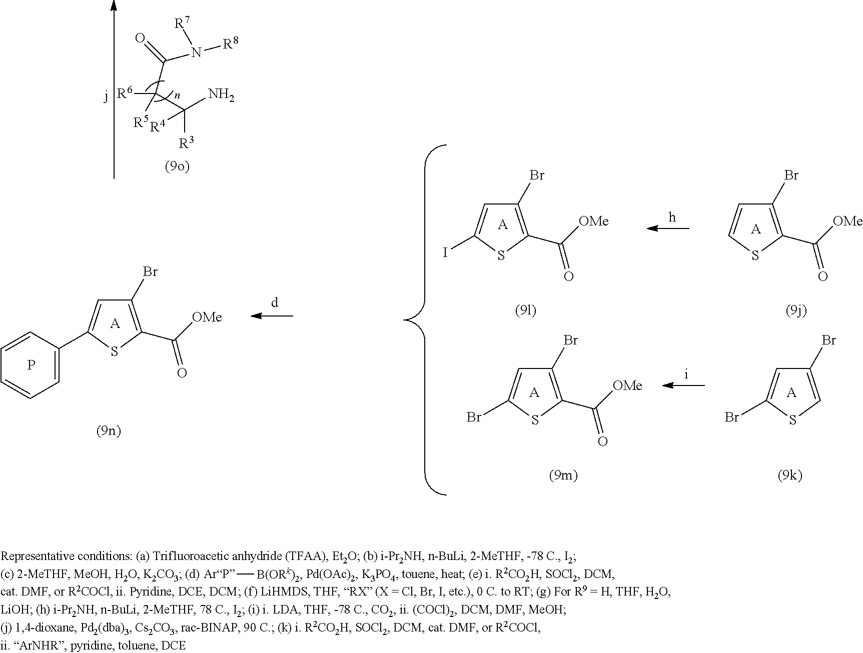

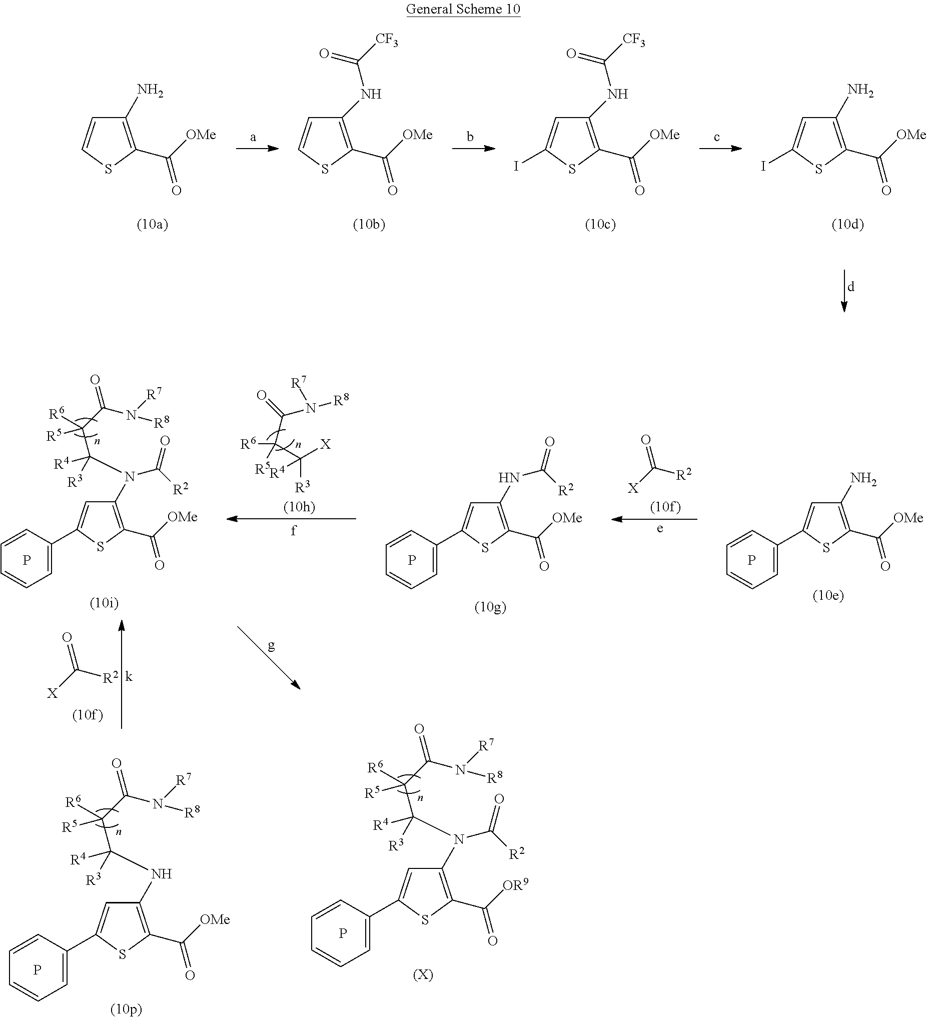

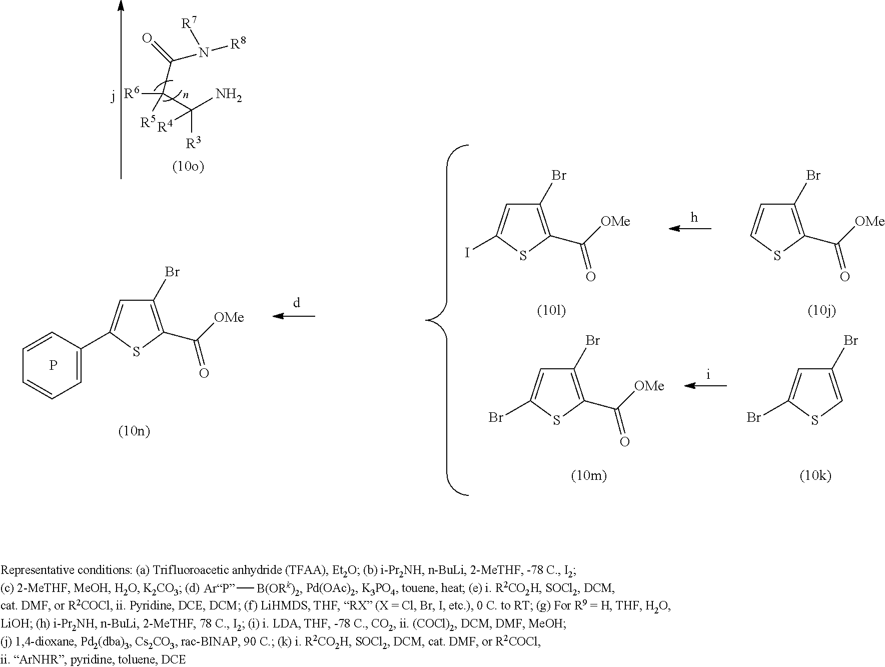

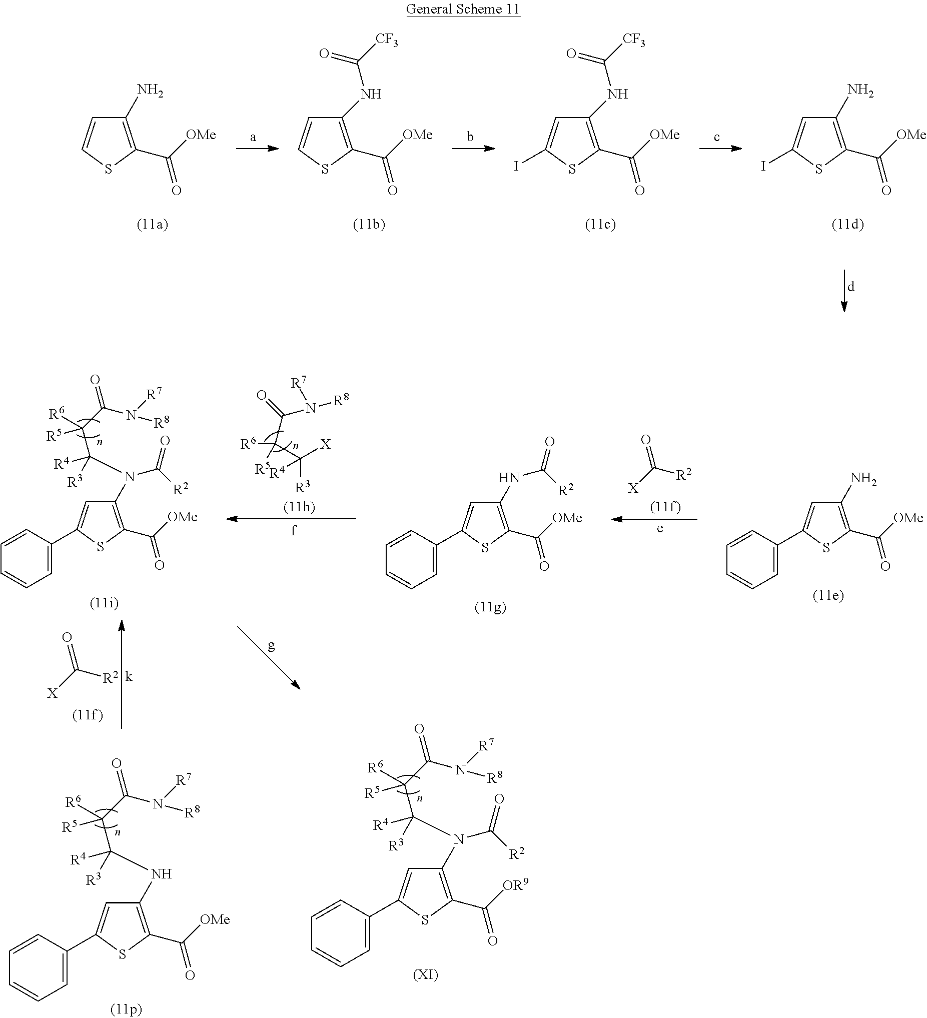

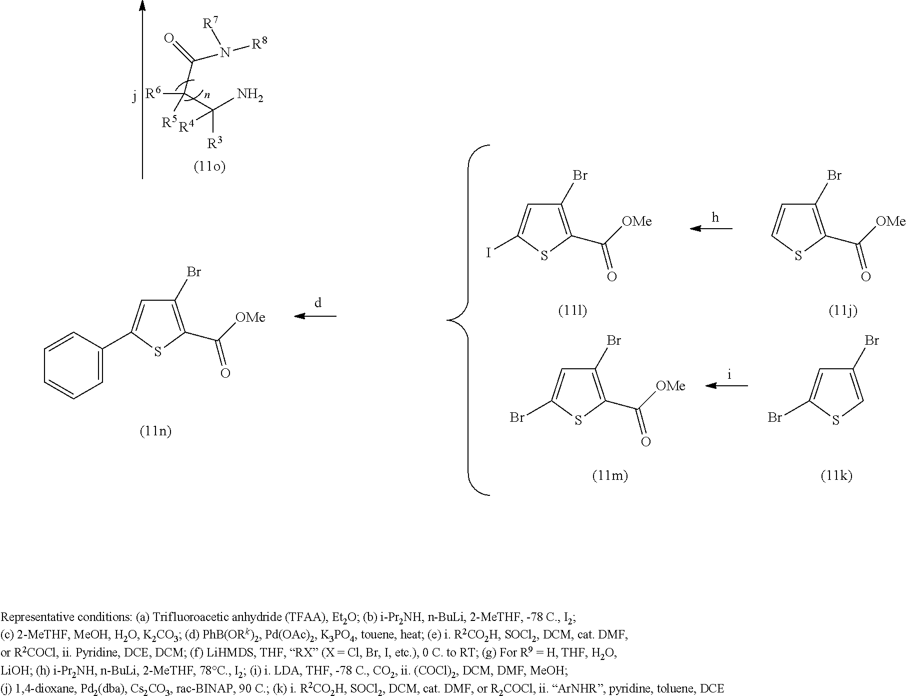

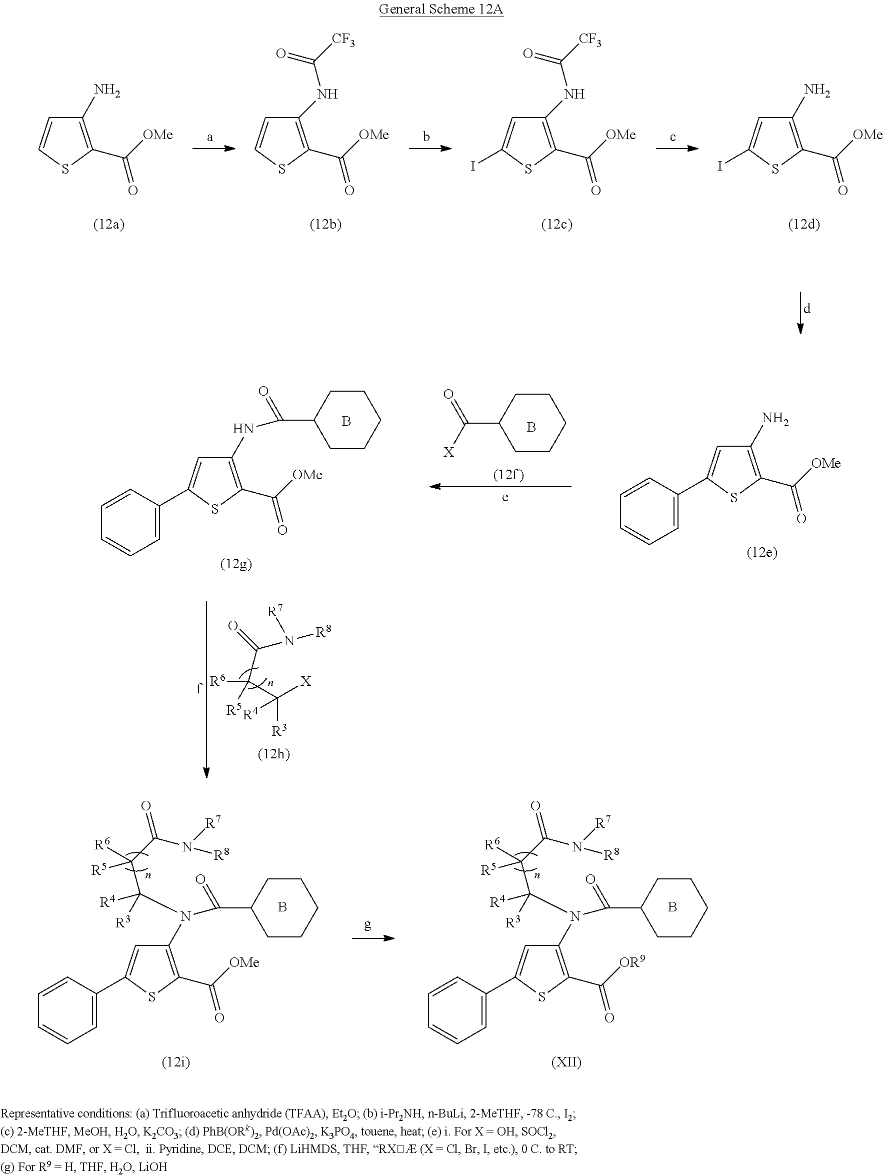

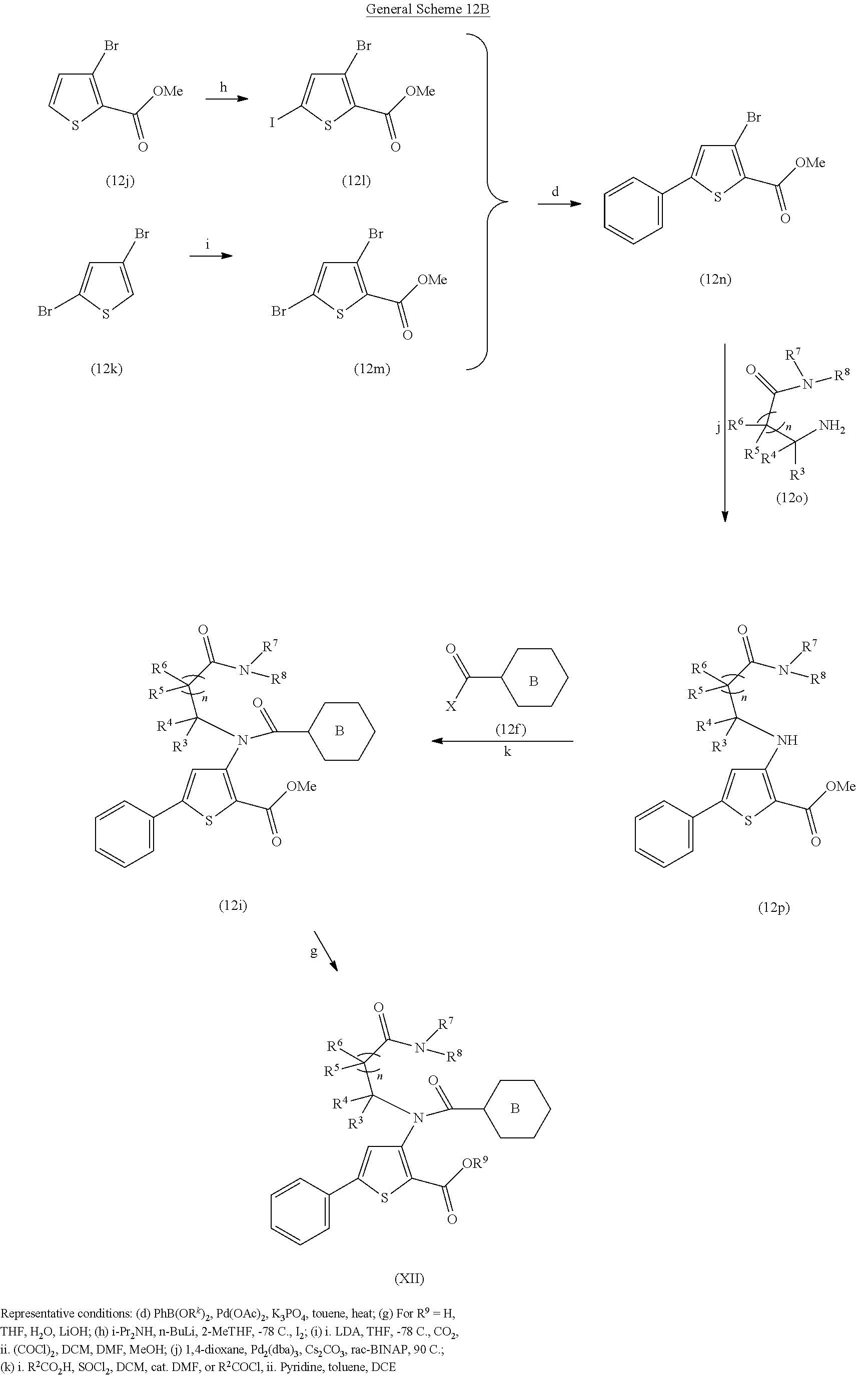

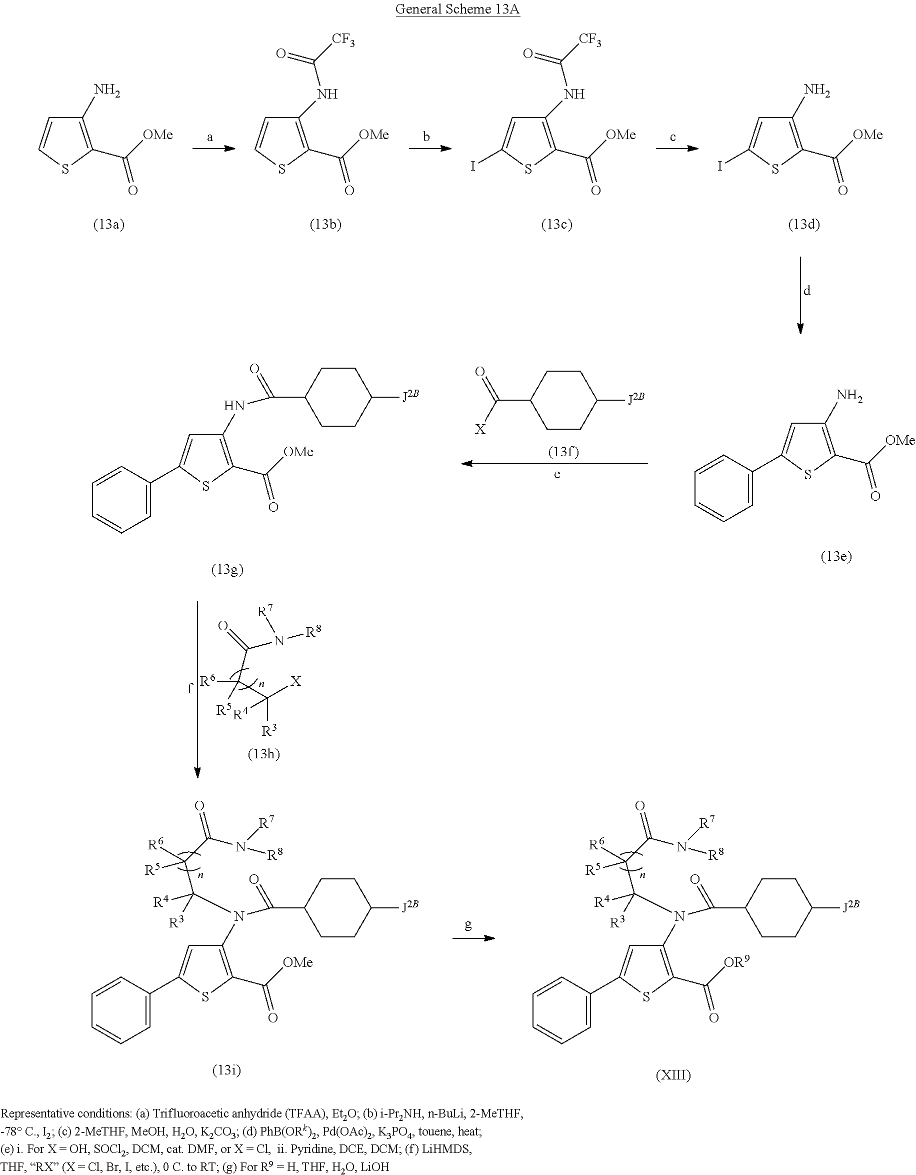

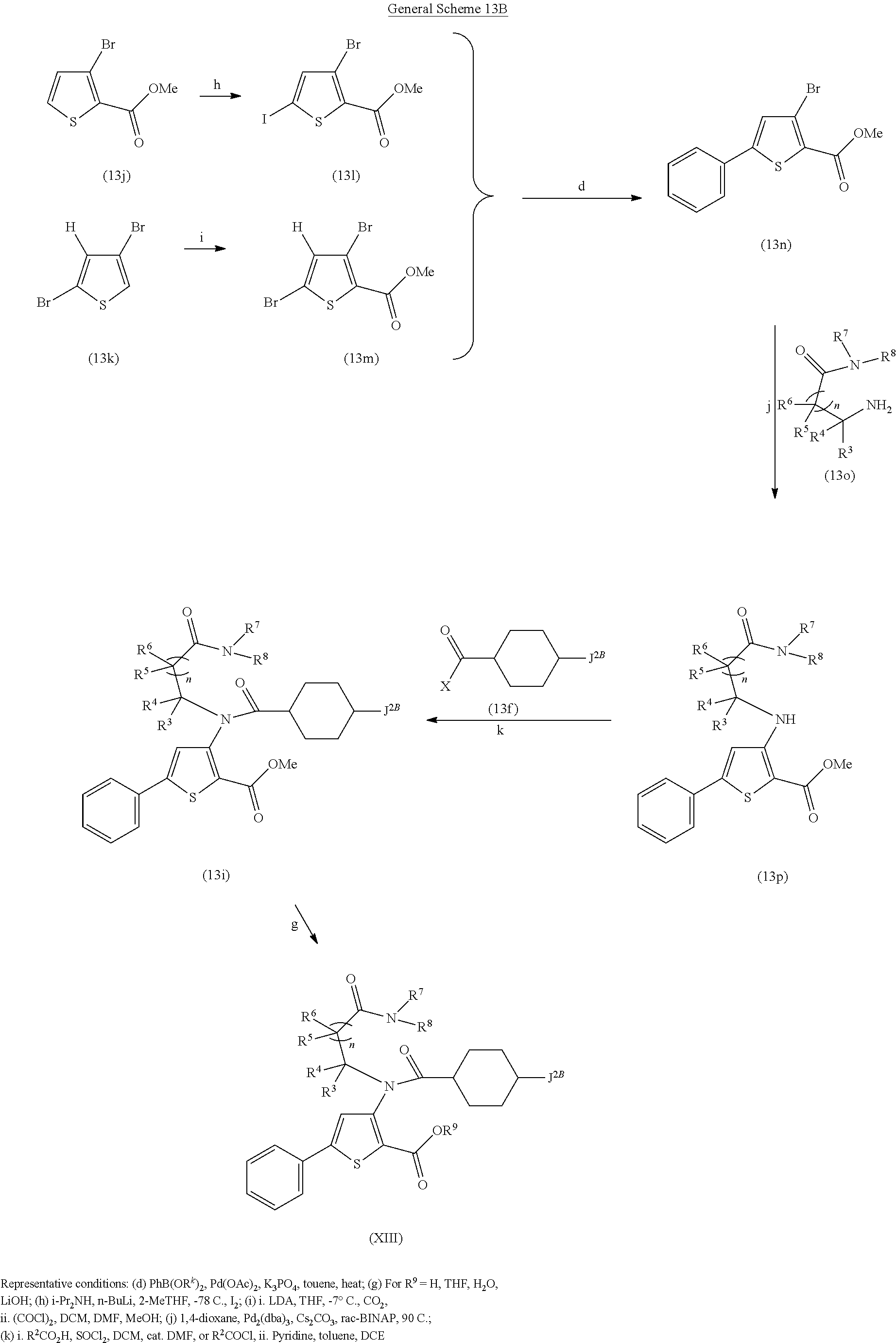

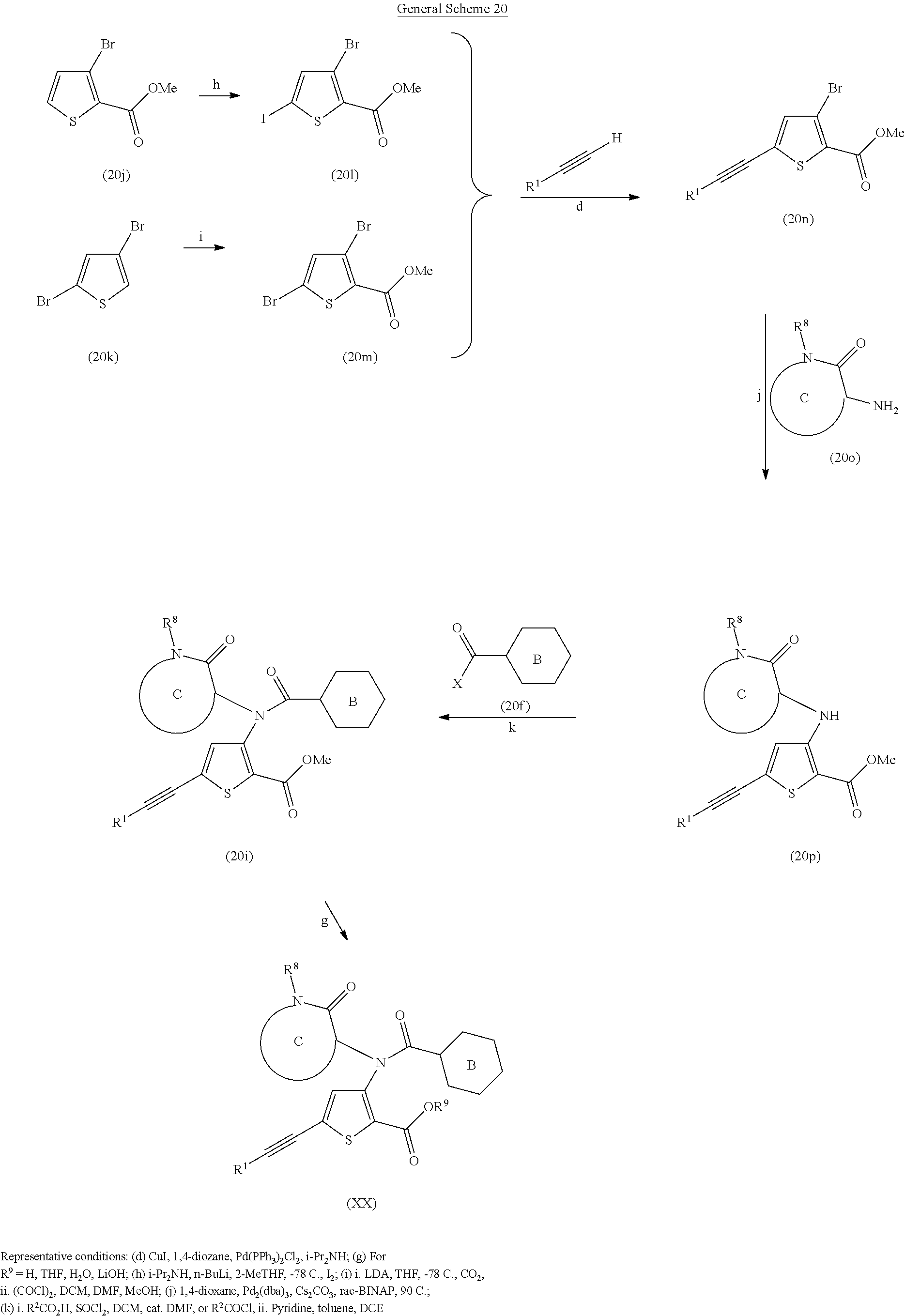

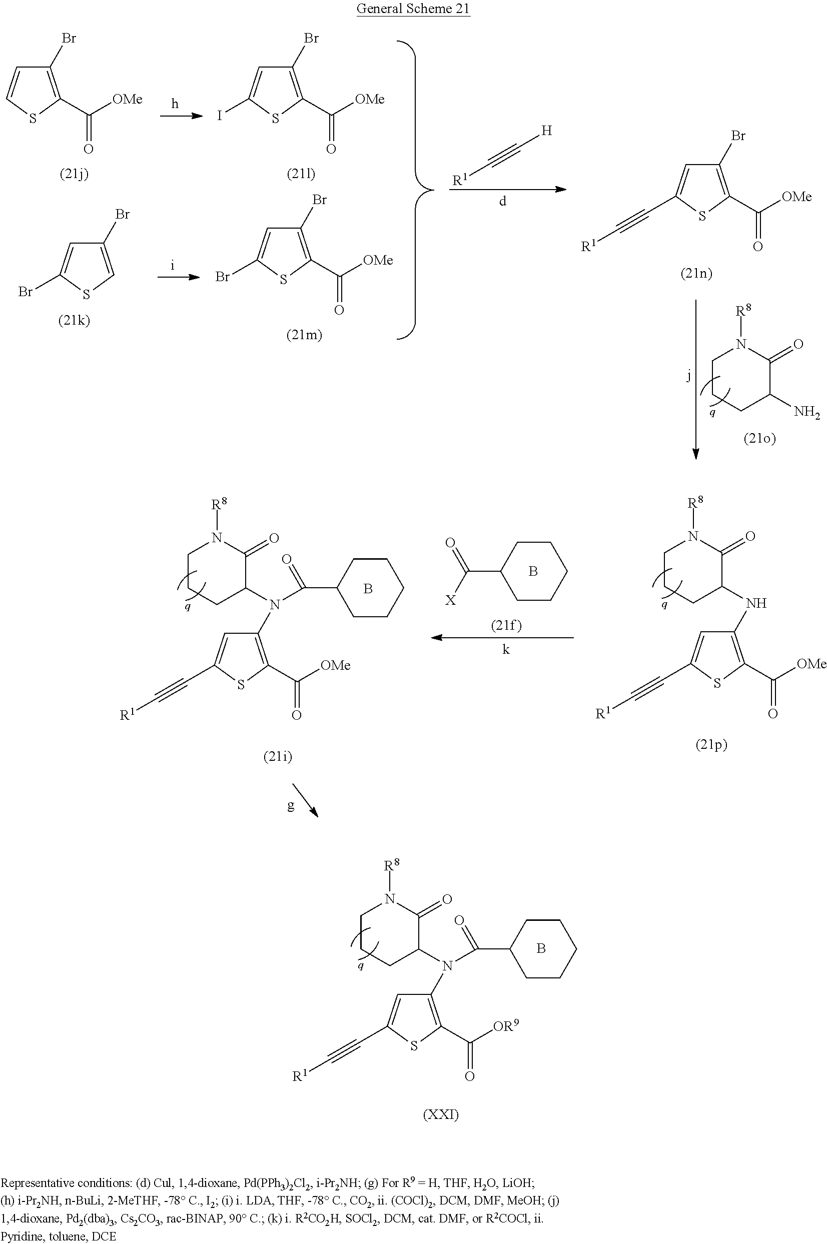

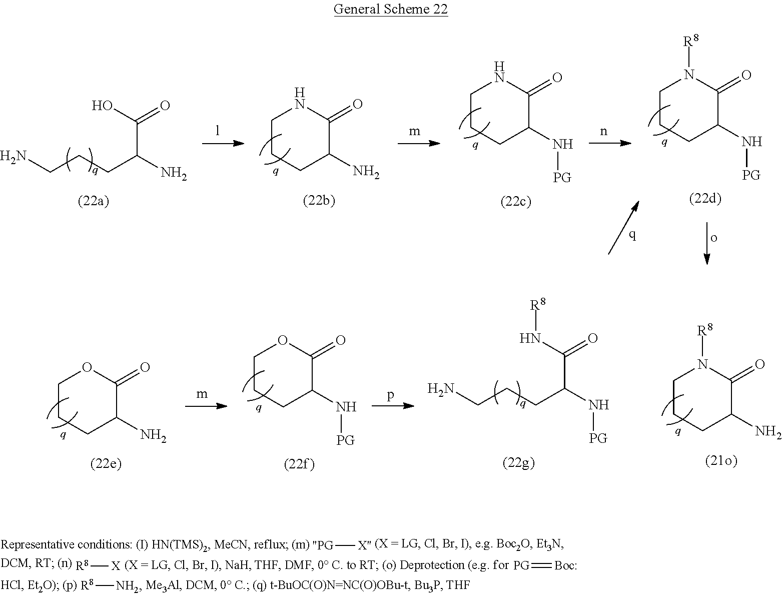

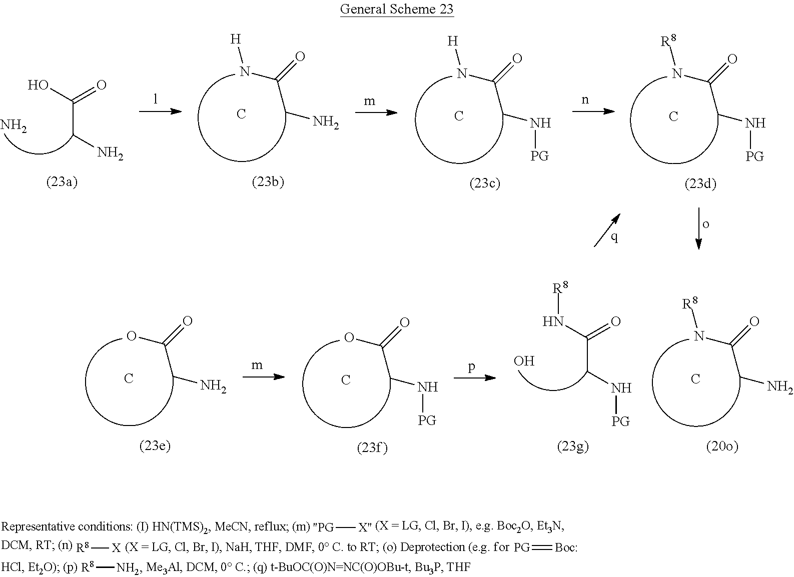

It will be appreciated by those skilled in the art that in the processes of the present invention certain functional groups such as hydroxyl or amino groups in the starting reagents or intermediate compounds may need to be protected by protecting groups. Thus, the preparation of the compounds described above may involve, at various stages, the addition and removal of one or more protecting groups. The protection and deprotection of functional groups is described in “Protective Groups in Organic Chemistry.” edited by J. W. F. McOmie, Plenum Press (1973) and “Protective Groups in Organic Synthesis,” 3rd edition, T. W. Greene and P. G. M. Wuts, Wiley Interscience, and “Protecting Groups,” 3rd edition, P. J. Kocienski, Thieme (2005)

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as illustrated generally below, or as exemplified by particular classes, subclasses, and species of the compounds described above. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. For example, if X is optionally substituted C₁-C₃alkyl or phenyl; X may be either optionally substituted C₁-C₃ alkyl or optionally substituted phenyl. Likewise, if the term “optionally substituted” follows a list, said term also refers to all of the substitutable groups in the prior list unless otherwise indicated. For example: if X is C₁-C₃alkyl or phenyl wherein X is optionally and independently substituted by J^{X}, then both C₁-C₃alkyl and phenyl may be optionally substituted by J^{X}. As is apparent to one having ordinary skill in the art, groups such as H, halogen, NO₂, CN, NH₂, OH, or OCF₃ would not be substitutable groups.

The phrase “up to”, as used herein, refers to zero or any integer number that is equal or less than the number following the phrase. For example, “up to 3” means any one of 0, 1, 2, and 3. As described herein, a specified number range of atoms includes any integer therein. For example, a group having from 1-4 atoms could have 1, 2, 3, or 4 atoms.

Selection of substituents and combinations of substituents envisioned by this invention are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “aliphatic” or “aliphatic group”, as used herein, means a straight-chain (i.e., unbranched), or branched, hydrocarbon chain that is completely saturated or that contains one or more units of unsaturation but is non-aromatic. Unless otherwise specified, aliphatic groups contain 1-10 aliphatic carbon atoms. In some embodiments, aliphatic groups contain 1-6 aliphatic carbon atoms. In other embodiments, aliphatic groups contain 1-4 aliphatic carbon atoms. Aliphatic groups may be linear or branched alkyl, alkenyl, or alkynyl groups. Specific examples include, but are not limited to, methyl, ethyl, isopropyl, n-propyl, sec-butyl, vinyl, n-butenyl, ethynyl, and tert-butyl and acetylene.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds. Each of the “alkyl”, “alkenyl” or “alkynyl” as used herein can be optionally substituted as set forth below. In some embodiments, the “alkyl” is C₁-C₆ alkyl or C₁-C₄ alkyl. In some embodiments, the “alkenyl” is C₂-C₆ alkenyl or C₂-C₄ alkenyl. In some embodiments, the “alkynyl” is C₂-C₆ alkynyl or C₂-C₄ alkynyl.

The term “cycloaliphatic” (or “carbocycle” or “carbocyclyl” or “carbocyclic”) refers to a non-aromatic carbon only containing ring system which can be saturated or contains one or more units of unsaturation, having three to fourteen ring carbon atoms. In some embodiments, the number of carbon atoms is 3 to 10. In other embodiments, the number of carbon atoms is 4 to 7. In yet other embodiments, the number of carbon atoms is 5 or 6. The term includes monocyclic, bicyclic or polycyclic, fused, spiro or bridged carbocyclic ring systems. The term also includes polycyclic ring systems in which the carbocyclic ring can be “fused” to one or more non-aromatic carbocyclic or heterocyclic rings or one or more aromatic rings or combination thereof, wherein the radical or point of attachment is on the carbocyclic ring. “Fused” bicyclic ring systems comprise two rings which share two adjoining ring atoms. Bridged bicyclic group comprise two rings which share three or four adjacent ring atoms. Spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle” (or “heterocyclyl,” or “heterocyclic” or “non-aromatic heterocycle”) as used herein refers to a non-aromatic ring system which can be saturated or contain one or more units of unsaturation, having three to fourteen ring atoms in which one or more ring carbons is replaced by a heteroatom such as, N, S, or O. In some embodiments, non-aromatic heterocyclic rings comprise up to three heteroatoms selected from N, S and O within the ring. In other embodiments, non-aromatic heterocyclic rings comprise up to two heteroatoms selected from N, S and O within the ring system. In yet other embodiments, non-aromatic heterocyclic rings comprise up to three heteroatoms selected from N and O within the ring system. In yet other embodiments, non-aromatic heterocyclic rings comprise up to two heteroatoms selected from N and O within the ring system. The term includes monocyclic, bicyclic or polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring. Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrofuranyl, tetrahydrothiophenyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl.

The term “aryl” (or “aryl ring” or “aryl group”) used alone or as part of a larger moiety as in “aralkyl”, “aralkoxy”, “aryloxyalkyl”, or “heteroaryl” refers to carbocyclic aromatic ring systems. The term “aryl” may be used interchangeably with the terms “aryl ring” or “aryl group”. “Carbocyclic aromatic ring” groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring” or “carbocyclic aromatic”, as it is used herein, is a group in which an aromatic ring is “fused” to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The terms “heteroaryl”, “heteroaromatic”, “heteroaryl ring”, “heteroaryl group”, “aromatic heterocycle” or “heteroaromatic group”, used alone or as part of a larger moiety as in “heteroaralkyl” or “heteroarylalkoxy”, refer to heteroaromatic ring groups having five to fourteen members, in which one or more ring carbons is replaced by a heteroatom such as, N, S, or O. In some embodiments, heteroaryl rings comprise up to three heteroatoms selected from N, S and O within the ring. In other embodiments, heteroaryl rings comprise up to two heteroatoms selected from N, S and O within the ring system. In yet other embodiments, heteroaryl rings comprise up to three heteroatoms selected from N and O within the ring system. In yet other embodiments, heteroaryl rings comprise up to two heteroatoms selected from N and O within the ring system. Heteroaryl rings include monocyclic heteroaromatic rings and polycyclic aromatic rings in which a monocyclic aromatic ring is fused to one or more other aromatic rings. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo”, “cyclic”, “cyclic group” or “cyclic moiety”, include mono-, bi-, and tri-cyclic ring systems including cycloaliphatic, heterocycloaliphatic, aryl, or heteroaryl, each of which has been previously defined.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “bridged bicyclic ring system” refers to a bicyclic heterocycloalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “bridge” refers to a bond or an atom or an unbranched chain of atoms connecting two different parts of a molecule. The two atoms that are connected through the bridge (usually but not always, two tertiary carbon atoms) are denotated as “bridgeheads”.

As used herein, the term “spiro” refers to ring systems having one atom (usually a quaternary carbon) as the only common atom between two rings.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein an optionally substituted aralkyl can be substituted on both the alkyl and the aryl portion. Unless otherwise indicated as used herein optionally substituted aralkyl is optionally substituted on the aryl portion.

In some embodiments, an aliphatic group and a heterocyclic ring may independently contain one or more substituents. Suitable substituents on the saturated carbon of an aliphatic group or of a non-aromatic heterocyclic ring are selected from those described above. Other suitable substitutents include those listed as suitable for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, wherein each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄ aliphatic groups of R* is unsubstituted.

In some embodiments, optional substituents on the nitrogen of a heterocyclic ring include those described above. Examples of such suitable substituents include —OH, —NH₂, —NH(C₁-C₄ alkyl), —N(C₁-C₄ alkyl)₂, —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇cycloalkyl, and C₃₋₇cyclo(haloalkyl). Other suitable substituents include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₂(Ph); optionally substituted —CH═CH(Ph); or an unsubstituted 5-6 membered heteroaryl or heterocyclic ring having one to four heteroatoms independently selected from oxygen, nitrogen, or sulfur, or, two independent occurrences of R⁺, on the same substituent or different substituents, taken together with the atom(s) to which each R⁺ group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group or the phenyl ring of R⁺are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄ aliphatic groups of R⁺ is unsubstituted.

In some embodiments, an aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from those described above. Specific examples include halogen, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen,

oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇cycloalkyl, and C₃₋₇ cyclo(haloalkyl). Other suitable substituents include: halogen; —R^{o}; —OR^{o}; —SR^{o}; 1,2-methylenedioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with Ro; —NO₂; —CN; —N(R^{o})₂; —NR^{o}C(O)R^{o}; —NR^{o}C(S)R^{o}; —NR^{o}C(O)N(R^{o})₂; —NR^{o}C(S)N(R^{o})₂; —NR^{o}CO₂R^{o}; —NR^{o}NR^{o}C(O)R^{o}; —NR^{o}NR^{o}C(O)N(R^{o})₂; —NR^{o}NR^{o}CO₂R^{o}; —C(O)C(O)Ro; —C(O)CH₂C(O)R; —CO₂R^{o}; —C(O)R^{o}; —C(S)R^{o}; —C(O)N(R^{o})₂; —C(S)N(R^{o})₂; —OC(O)N(R^{o})₂; —OC(O)R^{o}; —C(O)N(OR^{o})R^{o}; —C(NOR^{o}) R^{o}; —S(O)₂R^{o}; —S(O)₃R^{o}; —SO₂N(R^{o})₂; —S(O)R^{o}; —NR^{o}SO₂N(R^{o})₂; —NR^{o}SO₂R^{o}; —N(OR^{o})R^{o}; —C(═NH)—N(R^{o})₂; or —(CH₂)₀₋₂NHC(O)R^{o}; wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, two independent occurrences of R^{o}, on the same substituent or different substituents, taken together with the atom(s) to which each R^{o} group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group of R^{o} are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(haloC₁₋₄ aliphatic), or haloC₁₋₄ aliphatic, CHO, N(CO)(C₁₋₄ aliphatic), C(O)N(C₁₋₄ aliphatic), wherein each of the foregoing C₁₋₄ aliphatic groups of R^{o} is unsubstituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

The term “unsaturated”, as used herein, means that a moiety has one or more units of unsaturation.

As detailed above, in some embodiments, two independent occurrences of R^{o} (or R⁺, or any other variable similarly defined herein), may be taken together with the atom(s) to which each variable is bound to form a 5-8-membered heterocyclyl, aryl, or heteroaryl ring or a 3-8-membered cycloalkyl ring. Exemplary rings that are formed when two independent occurrences of R^{o} (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of OR^{o}

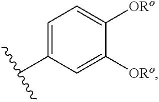

these two occurrences of R^{o} are taken together with the oxygen atoms to which they are bound to form a fused 6-membered oxygen containing ring:

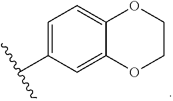 [SMILES: *c1ccc2c(c1)OCCO2]

It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

As used herein, an “amino” group refers to —NH₂.

The term “hydroxyl” or “hydroxy” or “alcohol moiety” refers to —OH.

As used herein, an “oxo” refers to ═O.

As used herein, the term “alkoxy”, or “alkylthio”, as used herein, refers to an alkyl group, as previously defined, attached to the molecule through an oxygen (“alkoxy” e.g., —O-alkyl) or sulfur (“alkylthio” e.g., —S-alkyl) atom.

As used herein, the terms “halogen”, “halo”, and “hal” mean F, Cl, Br, or I.

As used herein, the term “cyano” or “nitrile” refer to —CN or —C≡N.

The terms “alkoxyalkyl”, “alkoxyalkenyl”, “alkoxyaliphatic”, and “alkoxyalkoxy” mean alkyl, alkenyl, aliphatic or alkoxy, as the case may be, substituted with one or more alkoxy groups.

The terms “haloalkyl”, “haloalkenyl”, “haloaliphatic”, “haloalkoxy”, and “cyclo(haloalkyl)” mean alkyl, alkenyl, aliphatic, alkoxy, or cycloalkyl, as the case may be, substituted with one or more halogen atoms. This term includes perfluorinated alkyl groups, such as —CF₃ and —CF₂CF₃.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. In some embodiments, the cyanoalkyl is (NC)-alkyl-.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups, wherein the amino group is as defined above.

The terms “hydroxyalkyl”, “hydroxyaliphatic”, and “hydroxyalkoxy” mean alkyl, aliphatic or alkoxy, as the case may be, substituted with one or more —OH groups.

The terms “alkoxyalkyl”, “alkoxyaliphatic”, and “alkoxyalkoxy” mean alkyl, aliphatic or alkoxy, as the case may be, substituted with one or more alkoxy groups. For example, an “alkoxyalkyl” refers to an alkyl group such as (alkyl-O)-alkyl-, wherein alkyl has been defined above.

Unless otherwise specified, when the terms, for example, “aliphatic,” “alkyl” (including, for example, “alkoxyalkyl,” “hydroxyalkyl,” “haloalkyl,” “cyanoalkyl,” “aminoalkyl,”, etc.), “alkenyl,” “alkynyl,” “cycloaliphatic,” “heterocyclic,” “aryl,” “heteroaryl, “carbocyclic,” etc. are used without being specified by being optionally substituted, such terms mean unsubstituted.

The term “protecting group” and “protective group” as used herein, are interchangeable and refer to an agent used to temporarily block one or more desired functional groups in a compound with multiple reactive sites. In certain embodiments, a protecting group has one or more, or specifically all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

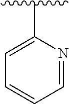 [SMILES: *c1ccccn1]

also represent

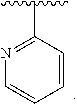 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays. Such compounds, especially deuterium (D) analogs, can also be therapeutically useful.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds described herein can exist in free form, or, where appropriate, as salts. Those salts that are pharmaceutically acceptable are of particular interest since they are useful in administering the compounds described above for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound, which are, within the scope of sound medical judgment, suitable for use in humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by, for example, 1) reacting the purified compound in its free-base form with a suitable organic or inorganic acid; and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by, for example, 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminium. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds described herein, the methods of the invention can be employed for preparing pharmaceutically acceptable solvates (e.g., hydrates) and clathrates of these compounds.

As used herein, the term “pharmaceutically acceptable solvate,” is a solvate formed from the association of one or more pharmaceutically acceptable solvent molecules to one of the compounds described herein. The term solvate includes hydrates (e.g., hemihydrate, monohydrate, dihydrate, trihydrate, tetrahydrate, and the like).

As used herein, the term “hydrate” means a compound described herein or a salt thereof that further includes a stoichiometric or non-stoichiometric amount of water bound by non-covalent intermolecular forces.

As used herein, the term “clathrate” means a compound described herein or a salt thereof in the form of a crystal lattice that contains spaces (e.g., channels) that have a guest molecule (e.g., a solvent or water) trapped within.

In addition to the compounds described herein, the methods of the invention can be employed for preparing pharmaceutically acceptable derivatives or prodrugs of these compounds.

A “pharmaceutically acceptable derivative or prodrug” includes any pharmaceutically acceptable ester, salt of an ester, or other derivative or salt thereof, of a compound described herein, which, upon administration to a recipient, is capable of providing, either directly or indirectly, a compound described herein or an inhibitorily active metabolite or residue thereof. Particularly favoured derivatives or prodrugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species.

As used herein and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds described herein that comprise —NO, —NO₂, —ONO, or —ONO₂ moieties. Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

Pharmaceutically acceptable prodrugs of the compounds described above include, without limitation, esters, amino acid esters, phosphate esters, metal salts and sulfonate esters.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds of the invention are provided in the form of a single enantiomer at least 95%, at least 97% and at least 99% free of the corresponding enantiomer.

In a further embodiment, the compounds of the invention are in the form of the (+) enantiomer at least 95% free of the corresponding (−) enantiomer.

In a further embodiment, the compounds of the invention are in the form of the (+) enantiomer at least 97% free of the corresponding (−) enantiomer.

In a further embodiment, the compounds of the invention are in the form of the (+) enantiomer at least 99% free of the corresponding (−) enantiomer.

In a further embodiment, the compounds of the invention are in the form of the (−) enantiomer at least 95% free of the corresponding (+) enantiomer.

In a further embodiment, the compounds of the invention are in the form of the (−) enantiomer at least 97% free of the corresponding (+) enantiomer.

In a further embodiment the compounds of the invention are in the form of the (−) enantiomer at least 99% free of the corresponding (+) enantiomer.

In some embodiments, the compounds of the invention are provided as pharmaceutically acceptable salts (e.g. Handbook of Pharmaceutical Salts Properties, Selection, and Use, Wiley, 2002, (P. Heinrich Stahl, Camille G. Wermuth, ed.)). As discussed above, such pharmaceutically acceptable salts can be derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium), alkaline earth metals (e.g. calcium, magnesium), ammonium, NR₄₊ (where R is C₁₋₄ alkyl) salts, choline and tromethamine.

In one embodiment of the invention, the pharmaceutically acceptable salt is a sodium salt.

In one embodiment of the invention, the pharmaceutically acceptable salt is a potassium salt.

In one embodiment of the invention, the pharmaceutically acceptable salt is a lithium salt.

In one embodiment of the invention, the pharmaceutically acceptable salt is a tromethamine salt.

In one embodiment of the invention, the pharmaceutically acceptable salt is an L-arginine salt.

In one embodiment of the invention, the pharmaceutically acceptable salt is a calcium salt.

It will be appreciated by those skilled in the art that the compounds of the invention described herein can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds of the invention described herein can exist in different solvate forms, for example hydrates. Solvates of the compounds of the invention may also form when solvent molecules are incorporated into the crystalline lattice structure of the compound molecule during the crystallization process.

The terms “subject,” “host,” or “patient” includes an animal and a human (e.g., male or female, for example, a child, an adolescent, or an adult). Preferably, the “subject,” “host,” or “patient” is a human.

In one embodiment, the present invention provides a method for treating or preventing a Flaviviridae viral infection in a host comprising administering to the host a therapeutically effective amount of at least one compound according to the invention described herein.

In one embodiment, the viral infection is chosen from Flavivirus infections. In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV).

In one embodiment, the methods of the invention are directed for treatment of HCV genotype 1 infection. In another embodiment, the HCV is genotype 1a or genotype 1b.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of a compound according to the invention described herein.

In one embodiment, there is provided a method for inhibiting or reducing the activity of viral polymerase in a host comprising administering a therapeutically effective amount of a compound according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In treating or preventing one or more conditions/diseases described above, the compounds described above can be formulated in pharmaceutically acceptable formulations that optionally further comprise a pharmaceutically acceptable carrier, adjuvant or vehicle.

In one embodiment, the present invention provides a pharmaceutical composition comprising at least one compound according to the invention described herein and at least one pharmaceutically acceptable carrier, adjuvant, or vehicle, which includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds described above, and pharmaceutically acceptable compositions thereof can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds (the compounds described above), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the active compounds administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the compounds described above and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds described above and pharmaceutically acceptable compositions thereof can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of a compound according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

In one embodiment, the present invention provides a pharmaceutical composition comprising at least one compound according to the invention described herein, and further comprising one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In another embodiment, there is provided a combination therapy of at least one compound according to the invention described herein in combination with one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

The additional agents for the compositions and combinations include, for example, ribavirin, amantadine, merimepodib, Levovirin, Viramidine, and maxamine.

In one combination embodiment, the compound and additional agent are administered sequentially.

In another combination embodiment, the compound and additional agent are administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851 A2, WO 01/85172 AI (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2 (Merck/Isis) and WO 02/057425 A2 (Merck/Isis).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190(Gilead), and PF-868,554 (Pfizer).

The term “viral NS5A inhibitor” as used herein means an agent that is effective to inhibit the function of the viral NS5A protease in a mammal. Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

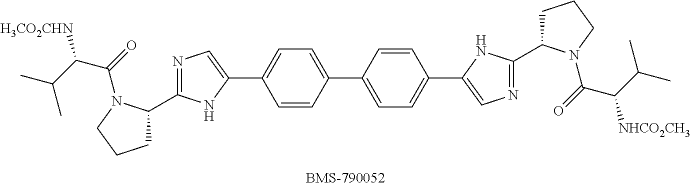

(Gao M. et al. Nature, 465, 96-100 (2010); nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class II interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir(Schering-Plough), VX-950/telaprevir(Vertex) and ITMN-B (InterMune), GS9132 (Gilead), TMC-435350(Tibotec/Medivir), ITMN-191 (InterMune), MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional agent is interferon alpha, ribavirin, silybum marianum, interleukine-12, amantadine, ribozyme, thymosin, N-acetyl cysteine or cyclosporin.

In one embodiment, the additional agent is interferon alpha 1A, interferon alpha 1 B, interferon alpha 2A, or interferon alpha 2B. Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-lntron™ regimen is 1.0 mg/kg/week subcutaneously for one year. The dose should be administered on the same day of the week.

When administered in combination with ribavirin, the recommended dose of PEG-lntron is 1.5 micrograms/kg/week.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments: viral serine protease inhibitor is HCV serine protease inhibitor; viral polymerase inhibitor is HCV polymerase inhibitor; viral helicase inhibitor is HCV helicase inhibitor.

In one embodiment, the present invention provides a pharmaceutical composition comprising at least one compound according to the invention described herein, one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, and at least one pharmaceutically acceptable carrier or excipient.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein for the manufacture of a medicament for treating or preventing a viral Flaviviridae infection in a host.

In one embodiment, the present invention provides the use of a compound according to the invention described herein for inhibiting or reducing the activity of viral polymerase in a host.

In a further embodiment, the composition or combination according to the invention further comprises at least one compound according to the invention described herein; one or more additional agents select from non-nucleoside HCV polymerase inhibitors (e.g., HCV-796), nucleoside HCV polymerase inhibitors (e.g., R7128, R1626, R1479), and HCV NS3 protease inhibitors (e.g., VX-950/telaprevir and ITMN-191); and interferon and/or ribavirin.

In one embodiment, the additional agent is interferon α 1A, interferon α 1B, interferon α 2A, or interferon α 2B, and optionally ribavirin.

In one embodiment, the present invention provides a method for treating or preventing a HCV viral infection in a host comprising administering to the host a combined therapeutically effective amounts of at least one compound according to the invention described herein, and one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin.

In one combination embodiment, the compound and additional agent are administered sequentially.

In another combination embodiment, the compound and additional agent are administered simultaneously.

In one embodiment, there is provided a method for inhibiting or reducing the activity of HCV viral polymerase in a host comprising administering to the host a combined therapeutically effective amounts of at least one compound of the invention, and one or more additional agents select from non-nucleoside HCV polymerase inhibitors (e.g., HCV-796) and nucleoside HCV polymerase inhibitors (e.g., R7128, R1626, R1479), interferon and ribavirin.

The combinations referred to above may conveniently be presented for use in the form of a pharmaceutical formulation and thus pharmaceutical formulations or compositions comprising a combination as defined above together with a pharmaceutically acceptable carrier therefore comprise a further aspect of the invention.

The individual components for use in the method of the present invention or combinations of the present invention may be administered either sequentially or simultaneously in separate or combined pharmaceutical formulations.

In one embodiment, the present invention provides the use of at least one compound of the invention, in combination with the use of one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, for the manufacture of a medicament for treating or preventing a HCV infection in a host.

When the compounds of the invention described herein are used in combination with at least one second therapeutic agent active against the same virus, the dose of each compound may be either the same as or differ from that when the compound is used alone. Appropriate doses will be readily appreciated by those skilled in the art.

The ratio of the amount of a compound according to the invention described herein administered relative to the amount of the additional agent (non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon or ribavirin) will vary dependent on the selection of the compound and additional agent.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXEMPLIFICATION

### Example 1

### Synthesis of Compounds of the Invention

The compounds according to the invention described herein can be prepared by any suitable method known in the art, for example, U.S. Pat. No. 6,881,741, US 2005/0009804, US 2006/0276533, WO 2002/100851, and WO 08/58393. Preparation details of some exemplary compounds are described below. Syntheses of certain exemplary compounds of the invention are described below. Generally, the compounds of the invention can be prepared as shown in those syntheses optionally with any desired appropriate modification.

### General Analytical Methods and Methodology for Synthesis and Characterization of Compounds

As used herein the term RT (min) refers to the LCMS retention time, in minutes, associated with the compound. NMR and Mass Spectroscopy data of certain specific compounds are summarized in Tables 1 and 2.

### Preparation of Compound 5

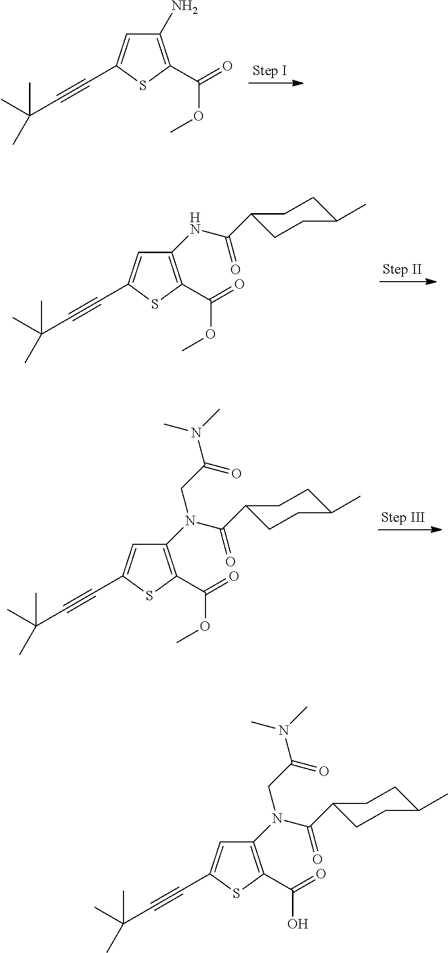

### Step I

### Trans-4-methylcyclohexyl carboxylic acid chloride

Oxalyl chloride (2M in dichloromethane, 17 mL) was added dropwise to a suspension of trans-4-methylcyclohexyl carboxylic acid (2.3 g, 16 mmol) in dichloromethane (5 mL) and DMF (0.1 mL). The reaction mixture was stirred for 3 h at room temperature. The volatiles were removed under reduced pressure to obtain the crude acid chloride which was used directly for the next reaction.

### 5-(3,3-dimethyl-but-1-ynyl)-3-[(trans-4-methyl-cyclohexanecarbonyl)-amino]-thiophene-2-carboxylic acid methyl ester

Trans-4-Methylcyclohexyl carboxylic acid chloride (2.54 g, 15.8 mmol) was added to a solution of 3-amino-5-(3,3-dimethyl-but-1-ynyl)-thiophene-2-carboxylic acid methyl ester (2.5 g, 10 mmol) in dichloroethane (20 mL). The resulting mixture was stirred for 16 h at 80° C. The reaction mixture was diluted with dichloromethane and then water was added. The organic layer was separated, dried (Na₂SO₄) and concentrated. The solid was purified by silica gel column chromatography using 20% EtOAc:hexane as eluent to obtain 5-(3,3-dimethyl-but-1-ynyl)-3-[(trans-4-methyl-cyclohexanecarbonyl)-amino]-thiophene-2-carboxylic acid methyl ester (3.37 g).

### Step II

### 5-(3,3-dimethyl-but-1-ynyl)-3-[dimethylcarbamoylmethyl-(trans-4-methyl-cyclohexanecarbonyl)-amino]-thiophene2-carboxylic acid methyl ester

In a 10 mL flask, 5-(3,3-dimethyl-but-1-ynyl)-3-[(trans-4-methyl-cyclohexanecarbonyl)-amino]thiophene-2-carboxylic acid methyl ester (100 mg, 0.277 mmol) was taken in DMF (1 mL) and cooled to 0° C., NaH was added (60%, 95 mg, 0.41 mmol) and stirred for 5 mins. 2-Chloro-N,N-dimethyl-acetamide (50 mg, 0.41 mmol) was added to the reaction mixture and stirred at rt overnight. The reaction mixture was cooled to 0° C. and added water (0.5 mL) and extracted between water and ethyl acetate. The organic layer was separated and dried (Na₂SO₄). The solvent was evaporated to obtain 5-(3,3-dimethyl-but-1-ynyl)-3-[dimethylcarbamoylmethyl-(trans-4-methyl-cyclohexanecarbonyl)-amino]-thiophene2-carboxylic acid methyl ester (49 mg, 42% yield).

### Step III

### 5-(3,3-Dimethyl-but-1-ynyl)-3-[dimethylcarbamoylmethyl-(trans-4-methyl-cyclohexanecarbonyl)-amino]-thiophene-2-carboxylic acid

To a solution of 5-(3,3-dimethyl-but-1-ynyl)-3-[dimethylcarbamoylmethyl-(trans-4-methyl-cyclohexanecarbonyl)-amino]-thiophene2-carboxylic acid methyl ester (52 mg, 0.12 mmol) in a 3:2:1 ratio of THF, methanol, water (1 mL) was added lithium hydroxide monohydrate (13 mg, 0.58 mmol). The resulting mixture was stirred at room temperature over night. Solvent was removed and the crude was purified by HPLC preparative using a Phenomenex AXIA Gemini 5u C18 110A 100 mm×30 mm column with a gradient of 39% to 69% acetonitrile: 3 mM aqueous HCl in 90 min and a flow rate of 12 mL/min. This afforded 5-(3,3-dimethyl-but-1-ynyl)-3-[dimethylcarbamoylmethyl-(trans-4-methyl-cyclohexanecarbonyl)-amino]-thiophene-2-carboxylic acid 5 (38 mg, 76% yield). LCMS [M+H]⁺: 433.05.

### Preparation of Compound 6

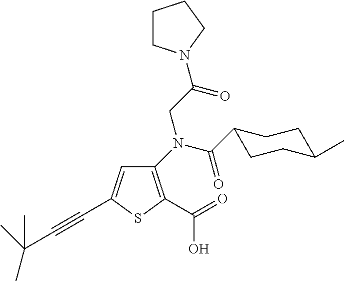 [SMILES: CC1CCC(C(=O)N(CC(=O)N2CCCC2)c2cc(C#CC(C)(C)C)sc2C(=O)O)CC1]

### 5-(3,3-Dimethyl-but-1-ynyl)-3-[(trans-4-methyl-cyclohexanecarbonyl)-(2-oxo-2-pyrrolidin-1-yl-ethyl)-amino]-thiophene-2-carboxylic acid

Compound 6 was prepared using a similar procedure as described above for the preparation of compound 5: LCMS [M+H]⁺: 459.01.

### Preparation of Compound 4

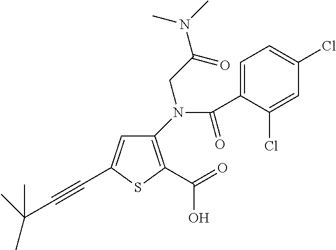 [SMILES: CN(C)C(=O)CN(C(=O)c1ccc(Cl)cc1Cl)c1cc(C#CC(C)(C)C)sc1C(=O)O]

### 3-[(2,4-Dichloro-benzoyl)-dimethylcarbamoylmethyl-amino]-5-(3,3-dimethyl-but-1-ynyl)-thiophene-2-carboxylic acid

Compound 4 was prepared using a similar procedure as described above for the preparation of compound 5: LCMS [M+H]⁺: 480.93.

### Preparation of Compound 1

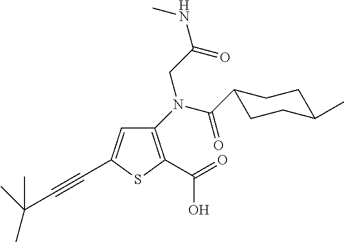 [SMILES: CNC(=O)CN(C(=O)C1CCC(C)CC1)c1cc(C#CC(C)(C)C)sc1C(=O)O]

### 5-(3,3-dimethylbut-1-ynyl)-3-[[2-(methylamino)-2-oxo-ethyl]-(trans-4-methylcyclohexanecarbonyl)amino]thiophene-2-carboxylic acid

Compound 1 was prepared using a similar procedure as described above for the preparation of compound 5: LCMS [M+H]⁺: 419.60.

### Preparation of Compound 2

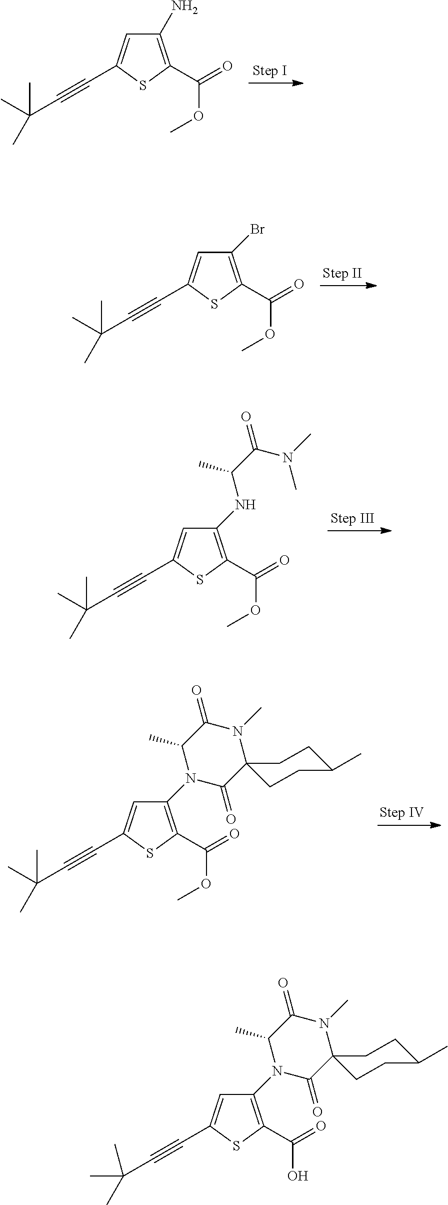

### Step I

A suspension of copper (II) bromide (1.74 g, 7.78 mmol) in acetonitrile was cooled to 0° C., then tert-butyl nitrite (1.24 mL, 10.4 mmol) was added, and the mixture was stirred for 15 min. 5-(3,3-Dimethyl-but-1-ynyl)-3-amino-thiophene-2-carboxylic acid methyl ester (1.76 g, 7.42 mmol) was added to the mixture in portions over 25 min. The mixture was allowed to warm up to room temperature, and was stirred at room temperature overnight. Then the mixture was evaporated to dryness, the residue was redissolved in 50 mL of CH₂Cl₂, 50 mL of HCl 1% were added to the mixture, and it was stirred at room temperature for 30 min. Organic fraction was separated, washed with brine, dried over Na₂SO₄ and evaporated to dryness to afford 2.905 g of 5-(3,3-dimethyl-but-1-ynyl)-3-bromo-thiophene-2-carboxylic acid methyl ester.

### Step II

To a solution of 5-(3,3-dimethyl-but-1-ynyl)-3-bromo-thiophene-2-carboxylic acid methyl ester (48 mg, 0.16 mmol) and (R)-2-amino-N,N-dimethyl-propionamide (37 mg, 0.32 mmol) in dry toluene (10 mL) were added cesium carbonate (230 mg, 0.048 mmol) and Pd(OAc)₂ (11 mg, 0.048 mmol). The mixture was deoxygenated by bubbling nitrogen through solution for 10 min. Then BINAP (40 mg, 0.064 mmol) was added to the mixture, and it was heated for 16 h at 90° C. under nitrogen. The mixture was diluted with ethyl acetate and filtered through celite washing with CH₂Cl₂. Filtrate was concentrated under reduced pressure, dried over Na₂SO₄ and purified by column chromatography on silica gel (methanol-EtOAc 0-10% gradient) to obtain 5-(3,3-dimethyl-but-1-ynyl)-3-((R)-1-dimethylcarbamoyl-ethylamino)-thiophene-2-carboxylic acid methyl ester (25 mg, 47%).

### Step III

5-(3,3-Dimethyl-but-1-ynyl)-3-((R)-1-dimethylcarbamoyl-ethylamino)-thiophene-2-carboxylic acid methyl ester (315 mg, 0.937 mmol) was acylated with trans-4-methylcyclohexyl carboxylic acid chloride as previously described (Example 3, step 1) to give desired product (418 mg, 97%).

### Step IV

### 5-(3,3-Dimethyl-but-1-ynyl)-3-[((R)-1-dimethylcarbamoyl-ethyl)-(trans-4-methyl-cyclohexanecarbonyl)-amino]-thiophene-2-carboxylic acid

The product from Step III (37 mg, 0.080 mmol) was hydrolysed with lithium hydroxide as described below to give compound 2 (14 mg, 41%): ¹H NMR (400 MHz, DMSO): δ 7.05 (s, 1H), 5.22 (q, 1H), 3.01 (s, 3H), 2.81 (s, 3H), 1.99 (t, 1H), 1.76-1.00 (m, 16H), 0.90 (d, 3H), 0.73 (d, 3H), 0.69-0.35 (m, 2H); LCMS [M+H]⁺: 447.2.

### Preparation of Compound 3

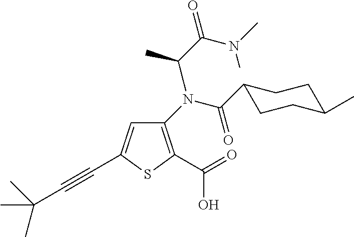 [SMILES: CC1CCC(C(=O)N(c2cc(C#CC(C)(C)C)sc2C(=O)O)[C@@H](C)C(=O)N(C)C)CC1]

### 5-(3,3-Dimethyl-but-1-ynyl)-3-[((S)-1-dimethylcarbamoyl-ethyl)-(trans-4-methyl-cyclohexanecarbonyl)-amino]-thiophene-2-carboxylic acid

Compound 3 was prepared using a similar procedure as described above for the preparation of compound 2: LCMS [M+H]⁺: 447.4.

### Preparation of Compound 7

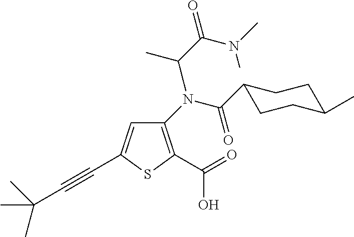 [SMILES: CC1CCC(C(=O)N(c2cc(C#CC(C)(C)C)sc2C(=O)O)C(C)C(=O)N(C)C)CC1]

### 5-(3,3-Dimethyl-but-1-ynyl)-3-[(1-dimethylcarbamoyl-ethyl)-(trans-4-methyl-cyclohexanecarbonyl)-amino]-thiophene-2-carboxylic acid

Compound 7 was prepared using a similar procedure as described above for the preparation of compound 2: LCMS [M+H]⁺: 446.99

### Alternative Preparation of Compound 6

### Step 1

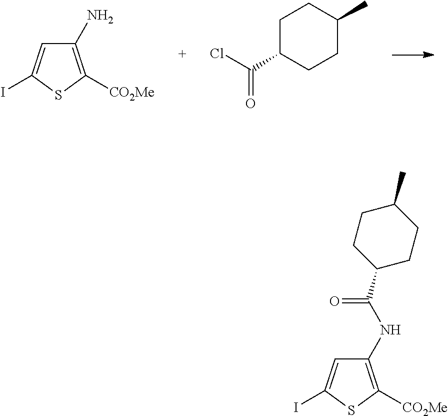

The acid chloride was prepared from the carboxylic acid by mixing with oxalyl chloride (1.1 eq.), in toluene (10 volume equivalents) at 0° C. with a catalytic amount (0.05 eq.) of DMF and slowly warming to RT (gas evolved). After stirring for 16 hours, the toluene was removed at 45° C. by rotary evaporation until the mixture was ca. two times the mass of the theoretical yield (50% by weight). The acid chloride could be stored at 0° C. under nitrogen for use as is in subsequent steps. Mixed commercially available methyl 3-amino-5-iodo-thiophene-2-carboxylate (5 g, 17.49 mmol) at 0° C. in 10% Pyridine/DCE/DCM (49.5 mL) and Pyridine (2.91 g, 2.97 mL, 36.7 mmol) as solvent, and then added the acid chloride (6.70 g, 21.0 mmol, 50% in toluene) dropwise. After 5 min, removed bath, and stirred as reaction came to RT for 1.25 hours. Worked up by adding brine, and then extraction with DCM (2×100 mL), combined and washed with 1N HCl (50 mL), washed with 1:1 1N NaOH (50 mL)/brine (50 mL); back extracted 1×, then dried over sodium sulfate, filtered and stripped to give a solid. Triturated the solid with hexanes, filtered and air dried to give desired product, 4.9 g (68%). Analysis by LCMS (60-98 MeOH/H₂O, formic acid modifier, 5/7 minutes, C18); RT=2.45 min, [M+H]=407.14.

### Step 2

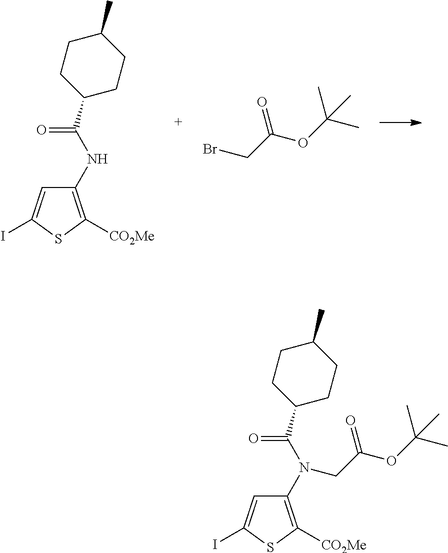

In a dry flask added starting amide (1 g, 2.3 mmol) and THF (9.5 mL), and placed under nitrogen. Cooled with an ice bath. Added (bis(trimethylsilyl)-amino)lithium (2.9 mL of 1M, 2.9 mmol), and stirred at 0° C. for 25 minutes. To the cold mixture was added tert-butyl 2-bromoacetate (546 mg, 2.80 mmol), and then the reaction was heated to 50° C., and stirred for 72 hours. Brine and EtOAc were added and the product was extracted in the organic layer, dried using sodium sulfate, filtered and dried to give 1.6 g. Purified by flash chromotography (SiO₂, ISCO instrument), using a gradient of EtOAc-Hexane, 0-20% over 10 column volumes (CV's), then at 20% for 2CV's. Obtained pure fractions, combined and stripped to give desired product (0.6 g, 48%) as a solid. Analysis by LCMS (60-98 MeOH/H₂O, formic acid modifier, 5/7 minutes, C18); RT=5.42 min, MH+=522.16.

### Step 3

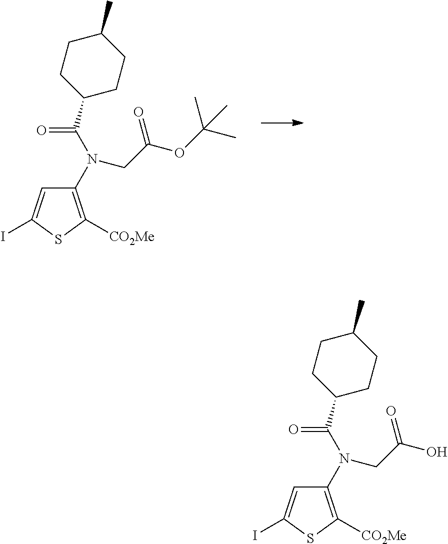 [SMILES: COC(=O)c1sc(I)cc1N(CC(=O)OC(C)(C)C)C(=O)[C@H]1CC[C@H](C)CC1 ; COC(=O)c1sc(I)cc1N(CC(=O)O)C(=O)[C@H]1CC[C@H](C)CC1]

Cooled a solution of starting t-Butyl ester (415 mg, 0.756 mmol) in DCM (2 mL) to 0° C. and then added TFA (3.0 mL, 39 mmol) in DCM (7 mL); 30% (TFA/DCM solution), allowed to come to RT/ON, removed the solvent and TFA using a rotary evaporator. After drying at high vacuum, obtained desired carboxylic acid as a reddish brown glass (435 mg, quantitative yield). Analysis by LCMS (60-98 MeOH/H₂O, formic acid modifier, 5/7 minutes, C18); RT=3.50 min, MH+=466.1.

### Step 4

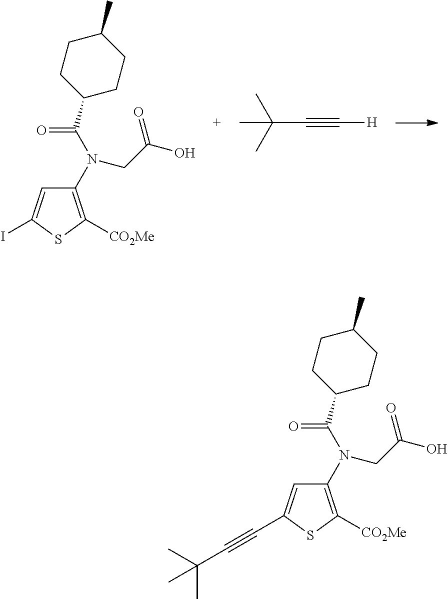

In a dry 100 mL flask under nitrogen atmosphere, mixed starting iodide (2.5 g, 4.3 mmol), in dioxane (54 mL), and cooled with an external ice bath and blew nitrogen over the reaction. Added iodocopper (41.2 mg, 0.216 mmol), followed by 3,3-dimethylbut-1-yne (800 μL, 6.70 mmol) and bubbled nitrogen for 5 min. Added dichloro-bis(triphenylphosphoranyl)palladium (165 mg, 0.234 mmol), and then the bath was removed and the reaction was stirred at RT overnight. Reaction was homogeneous. After about 16 hours, the reaction was checked and determined to be complete by tlc (20% MeOH/DCM). Added EtOAC (50 mL), and filter through fluoracil, rinse with EtOAc (3×25 mL), combined. Then eluted with 20% MeOH/EtOAc until product spot was very light. Combined fractions and stripped, product was a black solid glass. Added decolorizing carbon and stirred with ˜10% MeOH/EtOAc. Filter through celite, and removed solvent to obtain 2.9 g. Dissolved in DCM (250 mL), and washed with 1N HCl (100 mL), back-extract with DCM (100 mL), dried over sodium sulfate, filter and strip to give desired product (1.6 g, 88%). Analysis by LCMS (60-98 MeOH/H₂O, formic acid modifier, 5/7 minutes, C18), RT=4.63 min, MH+=420.3.

### Step 5

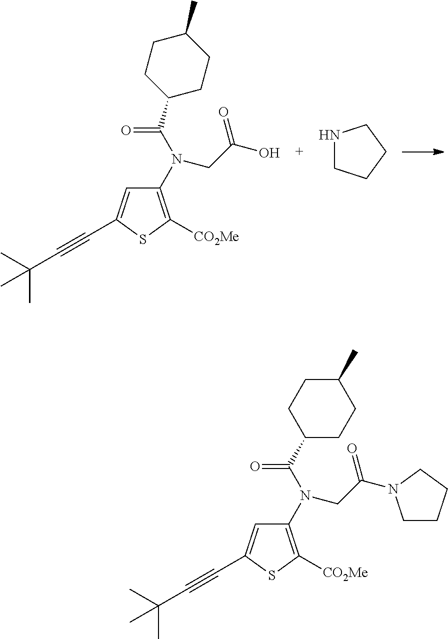 [SMILES: COC(=O)c1sc(C#CC(C)(C)C)cc1N(CC(=O)O)C(=O)[C@H]1CC[C@H](C)CC1 ; C1CCNC1 ; COC(=O)c1sc(C#CC(C)(C)C)cc1N(CC(=O)N1CCCC1)C(=O)[C@H]1CC[C@H](C)CC1]

Placed starting carboxylic acid (192 mg, 0.408 mmol) in a vial along with HBTU (1.66mL of 0.5 M, 0.830 mmol) (0.5 M solution in DMF prepared ahead of time and stored in a freezer). Added DIEA (134 mg, 181 μL, 1.04 mmol) followed by pyrrolidine (61.4 mg, 72.1 μL, 0.864 mmol), and the reaction stirred at RT o/n. Monitored the reaction by HPLC and found to be done. Aqueous workup by adding brine (60 mL), extracted with iPrOAc (2×60 mL); dried over sodium sulfate, filter and obtained crude product (240 mg). Purified by flash chromotography, (SiO₂, ISCO instrument, gold column, 12g); elute with 0-50% EtOAc/Hexanes, and isolated desired amide product (130 mg, 68%). Analysis by LCMS (60-98 MeOH/H₂O, formic acid modifier, 5/7 minutes, C18); RT=2.33 min, MH+=473.5.

### Step 6

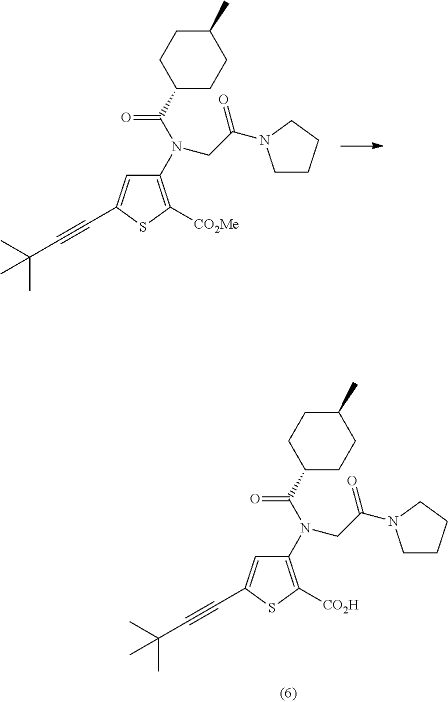 [SMILES: COC(=O)c1sc(C#CC(C)(C)C)cc1N(CC(=O)N1CCCC1)C(=O)[C@H]1CC[C@H](C)CC1 ; CC(C)(C)C#Cc1cc(N(CC(=O)N2CCCC2)C(=O)[C@H]2CC[C@H](C)CC2)c(C(=O)O)s1]

### Compound 6

Mixed starting methyl ester (130 mg, 0.275 mmol) dissolved in MeOH (5 mL), with NaOH (2N, 5 mL, 10 mmol), and the reaction was stirred at ambient temperature over-night. Checked the reaction by HPLC—and found no SM remaining. Stripped off MeOH, added 1N HCl (aq), 15 mL, and brine 15 mL. Extract 2×30 mL 1:1 diethyl ether-EtOAC (30 mL), dried over sodium sulfate, filtered and stripped to give a crude product. Purified on semi-prep HPLC (40-95% CH3CN/H₂O; 0.1% TFA); Pooled the homogeneous fractions with correct LCMS data to give desired compound 6 (54.5 mg, 42%). Analysis by LCMS (60-98 MeOH/H₂O, formic acid modifier, 5/7 minutes, C18); RT=1.62 min, MH+=459.46.

### Preparation of Compound 12

### Step 1

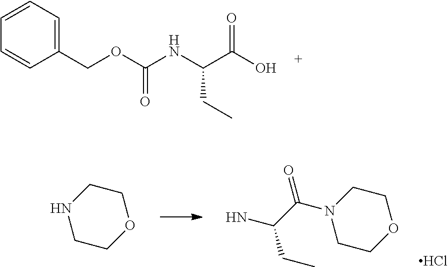 [SMILES: CC[C@H](NC(=O)OCc1ccccc1)C(=O)O ; C1COCCN1 ; CC[C@H](N)C(=O)N1CCOCC1 ; Cl]

### (S)-2-amino-1-morpholinobutan-1-one hydrochloride

(S)-2-(((benzyloxy)carbonyl)amino)butanoic acid (250 mg, 1.05 mmol) was mixed with HBTU (0.5M in DMF, 2.13 mL, 1.16 mmol), and DIEA (550 uL, 3.16 mmol), and morpholine (115 uL, 1.32 mmol), and stirred at RT for two hours, after which HPLC shows the reaction complete. Added brine (10 mL), and extract with EtOAc (2×10 mL). Pass organic extract through a plug of silica gel (2 g, bond-elute), and wash with excess EtOAc until no additional product elutes. Combined homogeneous fractions, and removed EtOAc under vacuum. Dissolved in MeOH (3 mL) and stirred under 1 atmosphere of hydrogen using a balloon in the presence of 10% Pd/C for a period of 16 hours. Filtered the reaction through a pad of celite, and wash with additional MeOH (3×5 mL). Starting material was consumed by tlc and a more polar spot that stained with ninhydrin confirmed product formation. Used as is without further purification or characterization in the next step.

### Step 2

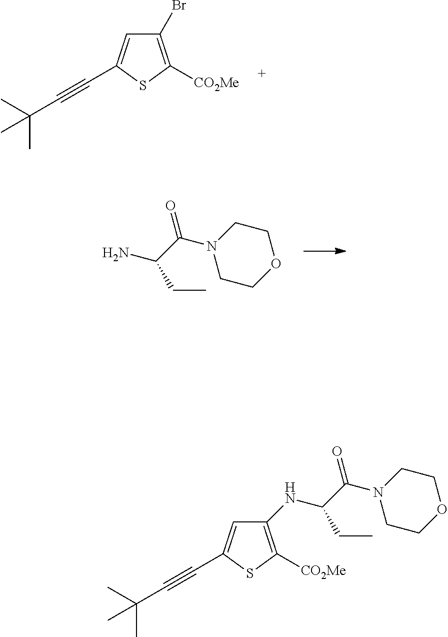

### (S)-methyl 5-(3,3-dimethylbut-1-yn-1-yl)-3-((1-morpholino-1-oxobutan-2-yl)amino)thiophene-2-carboxylate

Prepared as in compound 14, starting with methyl 3-bromo-5-(3,3-dimethylbut-1-ynyl)thiophene-2-carboxylate (100 mg, 0.329 mmol), purification in the same manner, gave the title compound as a solid (84 mg, 64%). Analysis by LCMS (60-98 MeOH/H₂O, formic acid modifier, 5/7 minutes, C18); RT=4.67 min, MH+=393.43. Used as is in the next step.

### Step 3

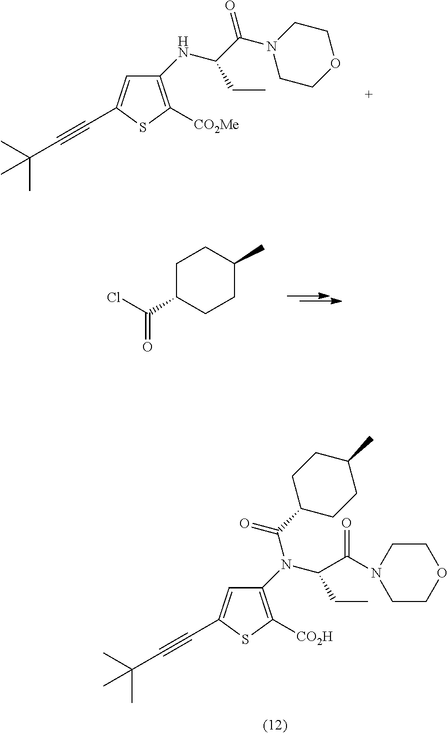

### Compound 12

Starting with (S)-methyl 5-(3,3-dimethylbut-1-yn-1-yl)-3-((1-morpholino-1-oxobutan-2-yl)amino)thiophene-2-carboxylate (84 mg, 0.21 mmol), prepared as in compound 14 to give compound 12 (14.7 mg, 13%). Analysis by LCMS (60-98 MeOH/H₂O, formic acid modifier, 5/7 minutes, C18); RT=5.60 min, MH+=416.46.

### Preparation of Compound 14

### Step 1

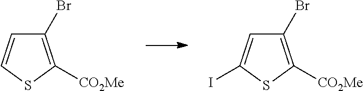

### Methyl 3-bromo-5-iodothiophene-2-carboxylate

n-BuLi (228 mL of 2.5 M, 570 mmol) was added drop wise to di-isopropylamine, and stirred for 30 minutes. The reaction mixture was cooled to −70° C., commercially available methyl 3-bromothiophene-2-carboxylate (105 g, 475.0 mmol) in 2-MeTHF (500 mL) was added drop wise over 1 h, after the addition the reaction mixture was stirred for 20 minutes. The reaction mixture warmed to −60° C., Iodine (132.6 g, 26.90 mL, 522.5 mmol) in 2-MeTHF (500 mL) was added drop wise over 1 h, maintained the internal temperature −60° C. and stirred at this temperature for 45 minutes, at which point HPLC and LCMS-analysis revealed consumption of the starting material. The reaction mixture was quenched with 1 M Na₂S₂O₃ solution (600 mL), and diluted with ethyl acetate (2 L), organic layer was separated, washed with 1 M Na₂S₂O₃ solution (500 mL), H₂O (500 mL), brine (500 mL), dried over Na₂SO₄, filter and concentrated under reduced pressure. The crude product was purified by silica-gel plug using 5-10% ethyl acetate in hexanes as eluent to afford methyl 3-bromo-5-iodo-thiophene-2-carboxylate 2 (75 g, 46% yield). This material was recrystallized from methanol to afford desired product (53 g, 32% yield) as a light yellow colored solid. Analysis by LCMS (10-90% CH₃CN/H₂O, formic acid modifier, 5 minutes, C18); RT=4.01 min, MH+=346.46, ¹H NMR (300 MHz, CDCl₃, ppm) 7.25 (s, 1H), 3.88 (s, 3H).

### Step 2

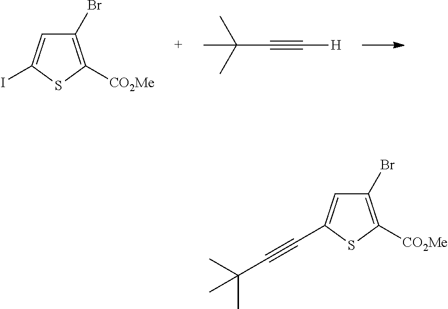

### Methyl 3-bromo-5-(3,3-dimethylbut-1-yn-1-yl)thiophene-2-carboxylate

In a dry flask under nitrogen atmosphere, were mixed in dimethylformamide (50 mL) methyl 3-bromo-5-iodothiophene-2-carboxylate (3.0 g, 8.6 mmol), 3,3-dimethylbut-1-yne (853 mg, 1.24 mL, 10.4 mmol), iodocopper (200 mg, 1.05 mmol), N,N-diethylethanamine (2.62 g, 3.62 mL, 25.9 mmol), and Tris(dba)Pd2 (86 mg, 0.094 mmol) at 0° C., and then stirred as reaction came to RT 12 hours. Monitor the reaction by HPLC for disappearance of starting material. Work up reaction by adding water-brine (1:1, 100 mL), and extraction with isopropyl-acetate (2×100 mL). Washed the combined extracts with saturated brine (1×100 mL); Dried over sodium sulfate, filtered and stripped to give 3.2 g of a yellow-brown oil. Purified by flash chromotography and obtained 2.46 g product as a solid (85%). Analysis by LCMS (10-90 CH₃CN/H₂O, formic acid modifier, 5 minutes, C18), R_{T}=5.60 min, MH+=301.09, ¹H NMR (300 MHz, CDCl₃, ppm) 7.05 (s, 1H), 3.89 (s, 3H), 1.32 (s, 9H).

### Step 3

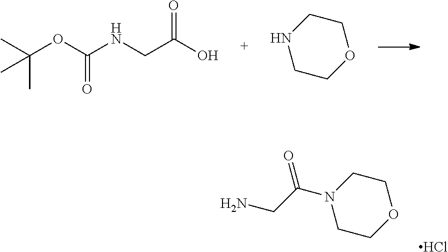

### 2-Amino-1-morpholinoethanone hydrochloride

Mixed 2-(tert-butoxycarbonylamino)acetic acid (175 mg, 1.0 mmol), morpholine (113 mg, 113 μL, 1.30 mmol), N-ethyl-N-isopropyl-propan-2-amine (388 mg, 522 μL, 3.00 mmol), (benzotriazol-1-yloxy-dimethylamino-methylene)-dimethyl-ammonium hexafluorophosphate (2.5 mL of 0.5 M, 1.25 mmol) (As a solution in DMF), in a reaction vial, and stirred at RT for 48 hours, and then judged complete after checking that the starting material was consumed as shown by LCMS. Added water (2 mL), and 2N NaOH (2 mL); Extract with 1:1 EtOAc-iPrOAc (3×4 mL); Remove solvent by passing nitrogen over the solution while heating to ˜35-50° C. over several hours. LCMS confirmed that the desired product was formed. MH+=245, MNa+=267. This material was treated with 6N HCl in isoproylalcohol at RT until all starting material was consumed. Removal of the solvent and excess HCl was carried out under vacuum, the material dried, and the product was used as is in the next step without further purification.

### Step 4

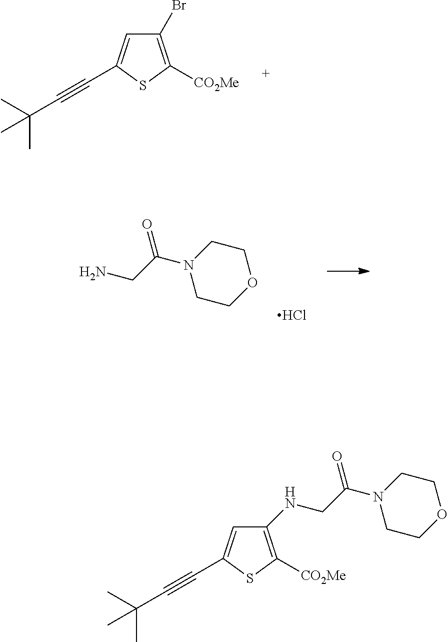

### Methyl 5-(3,3-dimethylbut-1-yn-1-yl)-3-((2-morpholino-2-oxoethyl)-amino)thiophene-2-carboxylate

Mixed methyl 3-bromo-5-(3,3-dimethylbut-1-ynyl)thiophene-2-carboxylate (2.00 g, 6.64 mmol), dicesium carbonate (5.19 g, 15.9 mmol), [1-(2-diphenylphosphanyl-1-naphthyl)-2-naphthyl]-diphenyl-phosphane (414 mg, 0.664 mmol), diacetoxypalladium (149 mg, 0.664 mmol), and a slight excess of amine-hydrochloride salt in toluene (60 mL) at RT. Degassed by bubbling in nitrogen gas for 10 minutes, then left standing at 80° C. for 16 hours. Reaction was monitored by HPLC, and found to have no starting material remaining. Poured the reaction mixture directly onto a plug of silca gel (˜25 g) that had been pre-wetted with hexanes. Elute and wash with hexanes (250 mL), then a step gradient; 50% EtOAc/Hexanes (3×250 mL); 60% EtOAc/Hexanes. Combined homogeneous fractions to give Methyl 5-(3,3-dimethylbut-1-yn-1-yl)-3-((2-morpholino-2-oxoethyl)-amino)thiophene-2-carboxylate as a glass (880 mg, 36%). Analysis carried out by LCMS (60-98%, MeOH/H₂O, formic acid modifier, 7 minutes, C18); RT=4.09 min, MH+=365.34.

### Step 5

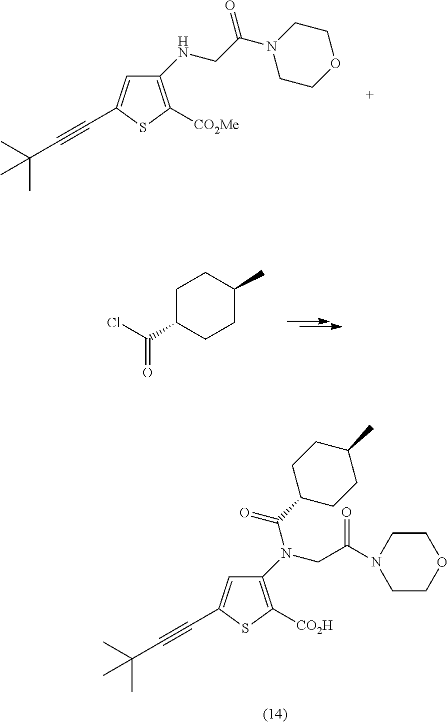

### Compound 14

Mixed 4-methylcyclohexanecarbonyl chloride (137 mg, 0.686 mmol), methyl 5-(3,3-dimethylbut-1-yn-1-yl)-3-((2-morpholino-2-oxoethyl)-amino)thiophene-2-carboxylate (50 mg, 0.14 mmol), and a catalytic amount of DMAP (1.6 mg, 0.014 mmol) in a mixture of 3% pyridine (110 uL, 1.37 mmol) in dichloro-ethane (3 mL). The mixture was placed in a microwave vial, and heated at 150° C. for 30 minutes. After the reaction was complete as judged by HPLC, added MeOH (2 mL), and 2N NaOH (2 mL), and heated the mixture at 70-80° C. for 2 hours followed by aqueous workup. To the mixture was added brine (10 mL), dilute HCl (2 mL, J^{2N}, diluted to about 10 mL), and extracted with EtOAC (2×50 mL). Dried over sodium sulfate, filtered and stripped. Purified by reverse phase HPLC (30-100%; MeOH/water/5 mMHCl). Obtained compound 14 (13.1 mg, 19%) as a dry solid. Analysis carried out by LCMS (60-98%, MeOH/H₂O, formic acid modifier, 7 minutes, C18); RT=5.21 min, MH+=475.45. ¹H NMR (300 MHz, CDCl₃) δ 6.93 (s, 1H), 5.01 (d, J=16.3 Hz, 1H), 4.03 (d, J=16.3 Hz, 1H), 3.80-3.60 (m, 5H), 3.51 (dd, J=13.5, 9.1 Hz, 2H), 2.32 (t, J=11.5 Hz, 1H), 1.86 (d, J=13.2 Hz, 1H), 1.64 (dd, J=31.7, 15.2 Hz, 4H), 1.34 (s, 9H), 0.78 (dd, J=28.1, 9.9 Hz, 5H).

### Preparation of Compound 13

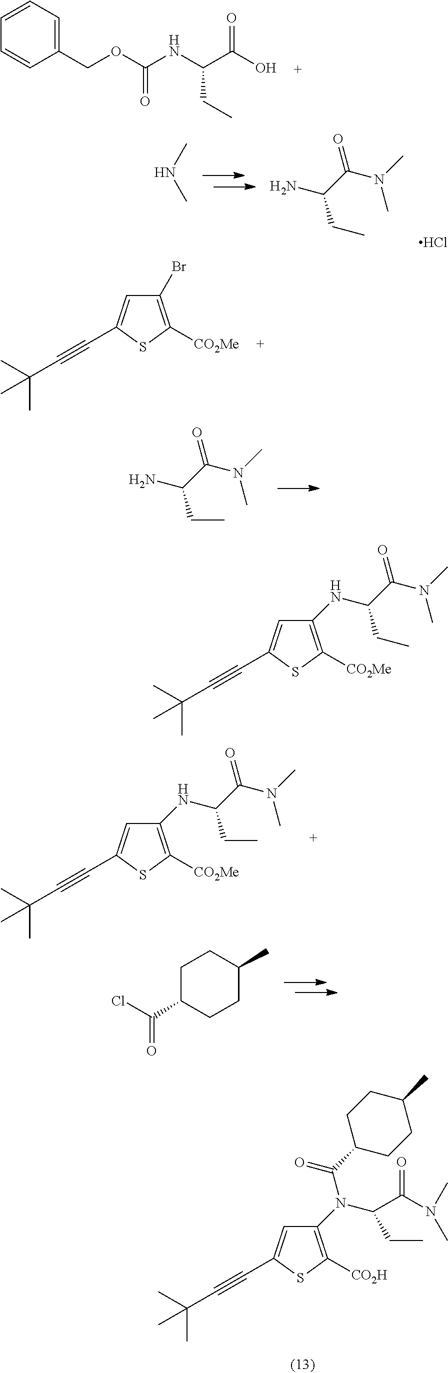

### Compound 13

Compound 13 was prepared starting with (S)-methyl 3-((1-(dimethylamino)-1-oxobutan-2-yl)amino)-5-(3,3-dimethylbut-1-yn-1-yl)thiophene-2-carboxylate (136 mg, 0.848 mmol), as described for compound 14 (29.6 mg, 30%). Analysis by LCMS (60-98 MeOH/H₂O, formic acid modifier, 5/7 minutes, C18); RT=5.28 min, MH+=461.

### Preparation of Compound 15

### Step 1

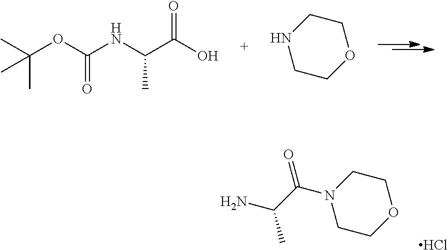

### (S)-2-amino-1-morpholinopropan-1-one hydrochloride

Prepared as in Compound 14 from commercially available (S)-2-((tert-butoxycarbonyl)-amino)propanoic acid (400 mg, 2.11 mmol), and obtained (S)-2-amino-1-morpholinopropan-1-one hydrochloride (446 mg, quantitative yield) as a solid, and used without further purification.

### Step 2

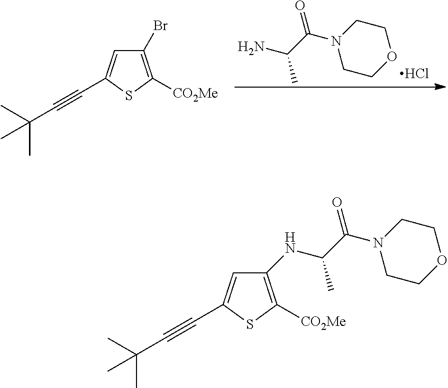 [SMILES: COC(=O)c1sc(C#CC(C)(C)C)cc1Br ; C[C@H](N)C(=O)N1CCOCC1 ; Cl ; COC(=O)c1sc(C#CC(C)(C)C)cc1N[C@@H](C)C(=O)N1CCOCC1]

### (S)-methyl 5-(3,3-dimethylbut-1-yn-1-yl)-3-((1-morpholino-1-oxopropan-2-yl)amino)thiophene-2-carboxylate

Prepared as in compound 14 from methyl 3-bromo-5-(3,3-dimethylbut-1-ynyl)thiophene-2-carboxylate (500 mg, 1.64 mmol), and purification in the same manner resulted in (S)-methyl 5-(3,3-dimethylbut-1-yn-1-yl)-3-((1-morpholino-1-oxopropan-2-yl)amino)thiophene-2-carboxylate as a solid (500 mg, 80%). Analysis by LCMS (60-98% MeOH/H₂O, formic acid modifier, 5/7 minutes, (C18); RT=4.33 min, MH+=379.35.

### Step 3

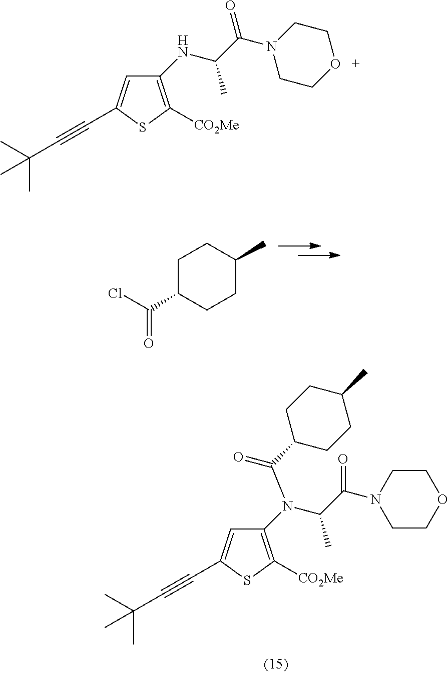

### Compound 15

Starting with (S)-methyl 5-(3,3-dimethylbut-1-yn-1-yl)-3-((1-morpholino-1-oxopropan-2-yl)amino)thiophene-2-carboxylate (461 mg, 0.977 mmol), prepared as described for compound 14 to give compound 15 (84.0 mg, 18%). Analysis by LCMS (40-80% CH₃CN/H₂O, formic acid modifier, 7 min, C18); RT=4.61 min, MH+=489.1.

### Preparation of Compound 16

### Step 1

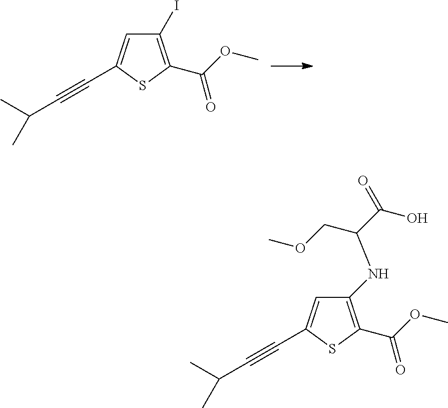 [SMILES: COC(=O)c1sc(C#CC(C)C)cc1I ; COCC(Nc1cc(C#CC(C)C)sc1C(=O)OC)C(=O)O]

### 3-Methoxy-2-((2-(methoxycarbonyl)-5-(3-methylbut-1-yn-1-yl)thiophen-3-yl)amino)propanoic acid

A mixture of methyl 3-iodo-5-(3-methylbut-1-yn-1-yl)thiophene-2-carboxylate (5 mmol), 2-amino-3-methoxypropanoic acid (7.5 mmol), K₂CO₃ (10 mmol), CuI (0.5 mmol) and L-proline (1 mmol) in 3 mL of DMSO was heated at 60° C. for 14 hrs. The cooled mixture was partitioned between water and ethyl acetate. The organic layer was separated, and the aqueous layer was extracted with ethyl acetate. The combined organic layers were washed with brine, dried over Na₂SO₄, and concentrated in vacuo. The residual oil was loaded on a silica gel column and eluted with 1/10 to 1/8 ethyl acetate/petroleum ether to give the desire product (26%). LCMS: 326.19 (MH+)

### Step 2

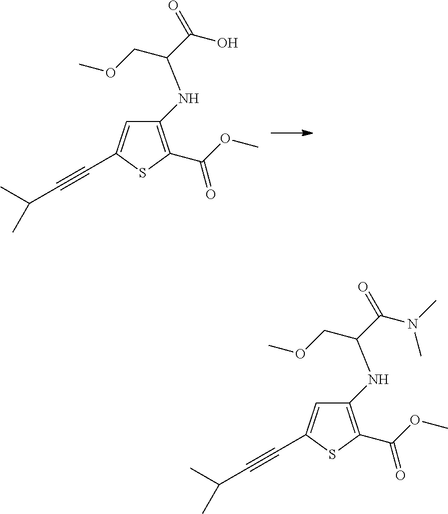 [SMILES: COCC(Nc1cc(C#CC(C)C)sc1C(=O)OC)C(=O)O ; COCC(Nc1cc(C#CC(C)C)sc1C(=O)OC)C(=O)N(C)C]

### Methyl 3-((1-(dimethylamino)-3-methoxy-1-oxopropan-2-yl)amino)-5-(3-methylbut-1-yn-1-yl)thiophene-2-carboxylate

To a solution of the starting acid (100 mg, 0.31 mmol) in dry DMF (2.000 mL) was added N-methylmethanamine (75 mg, 80 μL, 0.92 mmol), DIEA (238 mg, 321 μL, 1.84 mmol) and then HBTU (190 mg, 0.49 mmol). The reaction mixture was stirred at room temperature for 3 hrs, and then was diluted with EtOAc, the organic layer was washed with water and sat NaHCO₃ solution, dried over MgSO4, filtered, and evaporated under reduced pressure. The crude material was purified by silica gel chromatography (ISCO instrument) eluting with 5% EtOAc/Hex to 65% EtOAc/hex in 35 min to obtain the desired amide (95%). LCMS: 353.3 (MH+)

### Step 3

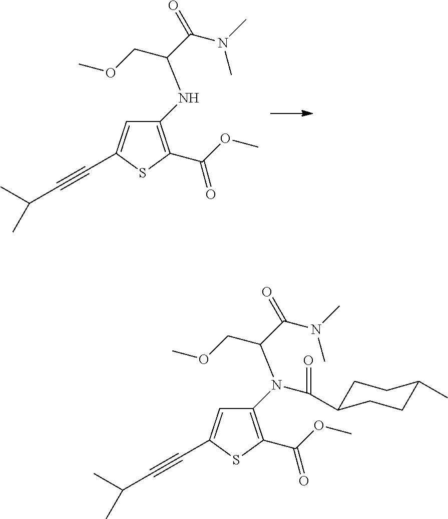 [SMILES: COCC(Nc1cc(C#CC(C)C)sc1C(=O)OC)C(=O)N(C)C ; COCC(C(=O)N(C)C)N(C(=O)C1CCC(C)CC1)c1cc(C#CC(C)C)sc1C(=O)OC]

A mixture of methyl 3-((1-(dimethylamino)-3-methoxy-1-oxopropan-2-yl)amino)-5-(3-methylbut-1-yn-1-yl)thiophene-2-carboxylate (60 mg, 0.17 mmol), (trans)-4-methylcyclohexanecarbonyl chloride (82 mg, 0.51 mmol), and DCE (3 mL) was heated in a sealed tube at 90° C. for 5 hrs. The crude material was evaporated with silica gel and loaded onto a silica column (ISCO instrument), eluting with 5% EtOAc/Hex to 65% EtOAc/hex in 30 min to afford an oil product (65%).

### Step 4

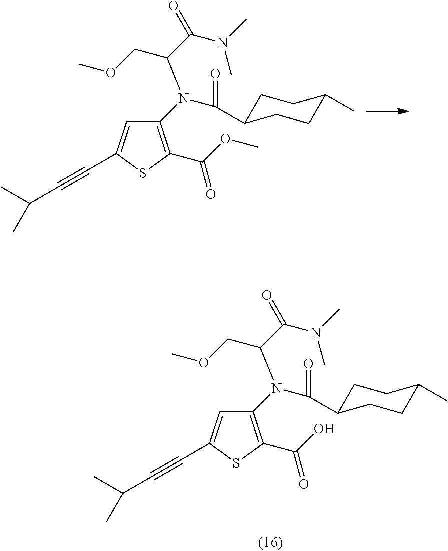

Compound 16

To a solution of starting ester (40 mg, 0.084 mmol) in THF (2 mL) was added water (2 mL) and LiOH (34 mg, 0.82 mmol). The mixture was stirred at RT until HPLC detected no more SM remained (about 6 hrs). The solution was acidified by adding 2N HCl until pH=2. The THF was removed by evaporation, the residue was taken up to 2N HCl and cooled in an ice bath. The resulting precipitated compound 16 was collected by filtration and washed with water (95%). LCMS: 463.36 (MH+).

### Preparation of Compound 21

### Step 1

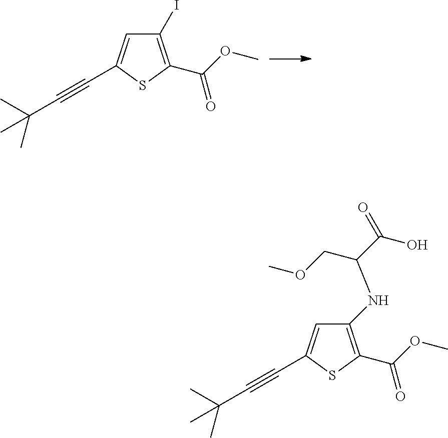 [SMILES: COC(=O)c1sc(C#CC(C)(C)C)cc1I ; COCC(Nc1cc(C#CC(C)(C)C)sc1C(=O)OC)C(=O)O]

### 2-((5-(3,3-dimethylbut-1-yn-1-yl)-2-(methoxycarbonyl)thiophen-3-yl)amino)-3-methoxypropanoic acid

A mixture of methyl 5-(3,3-dimethylbut-1-ynyl)-3-iodo-thiophene-2-carboxylate (1000 mg, 2.872 mmol), 2-amino-mixture of methyl 5-(3,3-dimethylbut-1-ynyl)-3-iodo-thiophene-2-carboxylate (1000 mg, 2.872 mmol), 2-amino-3-methoxy-propanoic acid (1.03 g, 8.62 mmol), K₂CO₃ (794 mg, 5.74 mmol), CuI (54.7 mg, 0.287 mmol) and (2S)-pyrrolidine-2-carboxylic acid (66.1 mg, 0.574 mmol) in DMSO (5.0 mL) was heated at 60° C. for 14 hrs. The cooled mixture was partitioned between water and ethyl acetate. The organic layer was separated, and the aqueous layer was extracted with ethyl acetate. The combined organic layers were washed with brine, dried over Na2SO4, and concentrated in vacuo. The residual oil was loaded on a silica gel column and eluted with 1/10 to 1/8 ethyl acetate/hexane to afford the desired product as an oil (46%). LCMS: 340.19 (MH+)

### Step 2

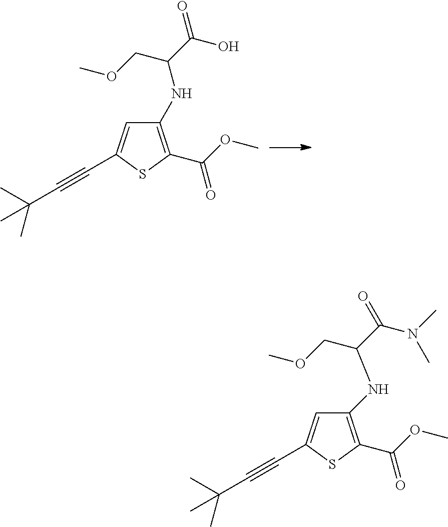 [SMILES: COCC(Nc1cc(C#CC(C)(C)C)sc1C(=O)OC)C(=O)O ; COCC(Nc1cc(C#CC(C)(C)C)sc1C(=O)OC)C(=O)N(C)C]

### Methyl 3-((1-(dimethylamino)-3-methoxy-1-oxopropan-2-yl)amino)-5-(3,3-dimethylbut-1-yn-1-yl)thiophene-2-carboxylate

To a solution of 2-[[5-(3,3-dimethylbut-1-ynyl)-2-methoxycarbonyl-3-thienyl]amino]-3-methoxy-propanoic acid (200 mg, 0.589 mmol) in dry DMF (4.0 mL) was added N-methylmethanamine (144 mg, 154 μL, 1.77 mmol), DIEA (457 mg, 616 μL, 3.54 mmol) and then HBTU (358 mg, 0.943 mmol). The mixture was stirred at RT for 3 hrs, then was diluted with EtOAc, the organic was washed with water, and sat NaHCO3 solution, dried over MgSO₄, evaporated with silica gel and purified by silica gel chromatography (ISCO instrument) eluting with 5% EtOAc/Hex to 65% EtOAc/hex in 35 min to afford the desired amide product as an oil (92%). LCMS: 367.32 (MH+); ¹H NMR (300 MHz, CDCl₃) δ 7.22 (d, J=8.2 Hz, 1H), 6.54 (s, 1H), 4.39 (dd, J=14.0, 6.9 Hz, 1H), 3.71 (s, 3H), 3.53 (ddd, J=15.1, 9.4, 6.4 Hz, 2H), 3.27 (s, 3H), 3.01 (s, 3H), 2.91 (s, 3H), 1.22 (s, 9H).

### Step 3

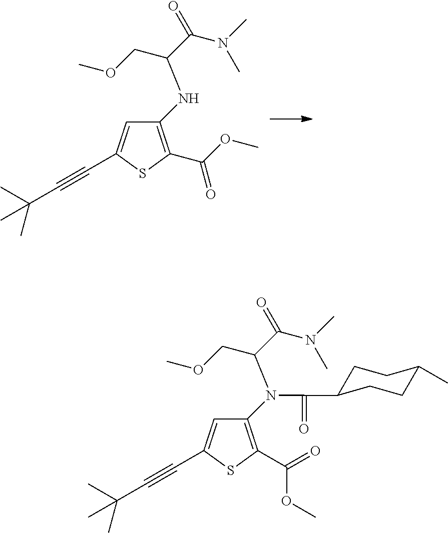 [SMILES: COCC(Nc1cc(C#CC(C)(C)C)sc1C(=O)OC)C(=O)N(C)C ; COCC(C(=O)N(C)C)N(C(=O)C1CCC(C)CC1)c1cc(C#CC(C)(C)C)sc1C(=O)OC]

To a solution of methyl 3-[[2-(dimethylamino)-1-(methoxymethyl)-2-oxo-ethyl]amino]-5-(3,3-dimethylbut-1-ynyl)thiophene-2-carboxylate (100 mg, 0.273 mmol) in DCE (2.000 mL) was added pyridine (25.9 mg, 26.5 μL, 0.328 mmol) and followed by adding 4-methylcyclohexanecarbonyl chloride (65.8 mg, 0.410 mmol); the mixture was heated at 130° C. fin microwave for 1200 seconds. The crude product was purified by silica gel chromatography (ISCO instrument) eluting with 5% EtOAc/Hex to 65% EtOAc/hex in 30 min to afford an oil product (70%); LCMS: 491.39 (MH+).

### Step 4

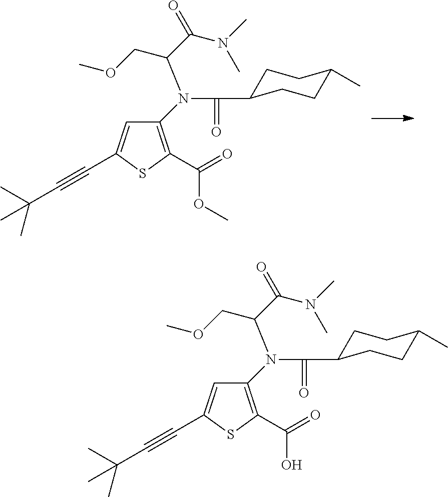

Racemic 21

To a solution of starting ester (120 mg, 0.24 mmol) in water (2.3 mL) and THF (2.3 mL) was added lithium hydroxide (58 mg, 2.4 mmol), the mixture was stirred at RT for 12 hrs, and then was acidified with 6N HCl to pH=2; the THF was removed by evaporation, the precipitated racemic 21 was collected by filtration and was purified by Gilson HPLC. LCMS: 477.39 (MH+).

### Step 5

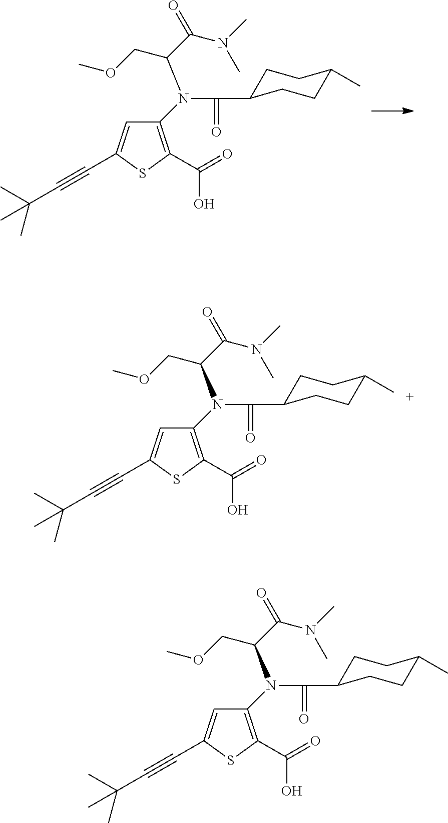

### 3-[[(1S)-2-(dimethylamino)-1-(methoxymethyl)-2-oxo-ethyl]-(trans-4-methylcyclohexanecarbonyl)amino]-5-(3,3-dimethylbut-1-ynyl)thiophene-2-carboxylic acid

The two enantiomers were separated using SFC to give 21: Whelk-O column (10×250); Mobile phase: 15% EtOH, 85% CO₂; Flow rate: 10 mL/min; pressure: 100 bar.

### Preparation of Compound 23

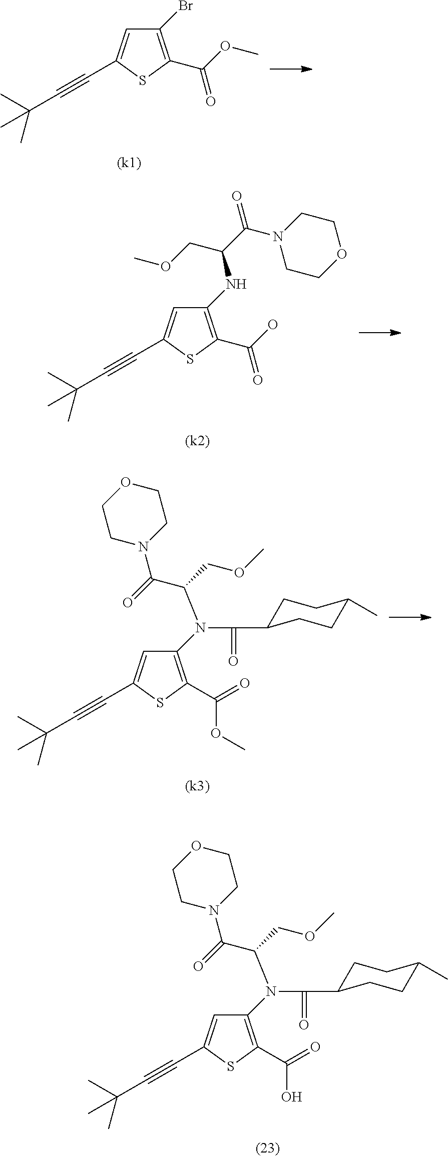

### (S)-Methyl 5-(3,3-dimethylbut-1-yn-1-yl)-3-((3-methoxy-1-morpholino-1-oxopropan-2-yl)amino)thiophene-2-carboxylate (Compound (k2))

Anhydrous 1,4-dioxane used in the reaction was deoxygenated by bubbling nitrogen for 30 mins. A mixture of methyl 3-bromo-5-(3,3-dimethylbut-1-ynyl)thiophene-2-carboxylate (compound (k1), 515.7 mg, 1.71 mmol), (S)-2-amino-3-methoxy-1-morpholinopropan-1-one (500 mg, 2.23 mmol), dicyclohexyl-[2-(2,6-dimethoxyphenyl)phenyl]phosphane (S-phos, 70.3 mg, 0.171 mmol) and cesium carbonate (1.67 g, 5.14 mmol) were taken into 15 mL of 1,4-dioxane and the mixture was further degassed with argon for 10 minutes. The catalyst Pd₂(dba)₃ (78 mg, 0.086 mmol) was added to the mixture and the reaction was sealed and heated at 90° C. for 18 hrs. The reaction was cooled to room temperature, diluted with EtOAc, and washed with water and brine. The organic layer was dried over anhydrous MgSO4, filtered, evaporated. The crude material was absorbed onto silica gel and purified by silica gel chromatography (ISCO instrument) eluting with 0% EtOAc/Hex to 35% EtOAc/Hex in min (57%). LCMS: 409.48 (MH+).

### Compound (k3)

A mixture of (S)-methyl 5-(3,3-dimethylbut-1-yn-1-yl)-3-((3-methoxy-1-morpholino-1-oxopropan-2-yl)amino)thiophene-2-carboxylate (150 mg, 0.37 mmol), 4-methylcyclohexanecarbonyl chloride (177 mg, 1.10 mmol), and pyridine (58.1 mg, 59.4 μL, 0.734 mmol) was taken into 1,2-dichloroethane (7.5 mL) and heated in a sealed tube at 90° C. for 14 hrs. Methanol was added to the reaction followed by the evaporation of solvent. The resulting residue was purified by silica gel chromatography (ISCO instrument) eluting with 1% EtOAc/Hex to 70% in 35 min to give an oil product (82%). LCMS: 533.36 (MH+).

### Compound 23

To a solution of (k3) (160 mg, 0.30 mmol) in water (3 mL) and THF (3 mL) was added lithium hydroxide (72 mg, 3.0 mmol). The mixture was stirred at RT for 12 hrs. The reaction was acidified with 6N HCl and the THF was removed by evaporation. The resulting precipitate was filtered, washed with water and dried to afford compound 23 (95%). LCMS: 519.41 (MH+). ¹H NMR (300 MHz, DMSO) δ 13.43 (s, 1H), 7.28 (s, 0.5H), 7.07 (s, 0.5H), 5.83 (t, J=7.2 Hz, 0.5H), 5.41 (t, J=6.7 Hz, 0.5H), 3.79-2.89 (m, 14H), 2.11-1.00 (m, 17H), 0.87-0.39 (m, 4H).

### Preparation of Compound 26

### Step 1

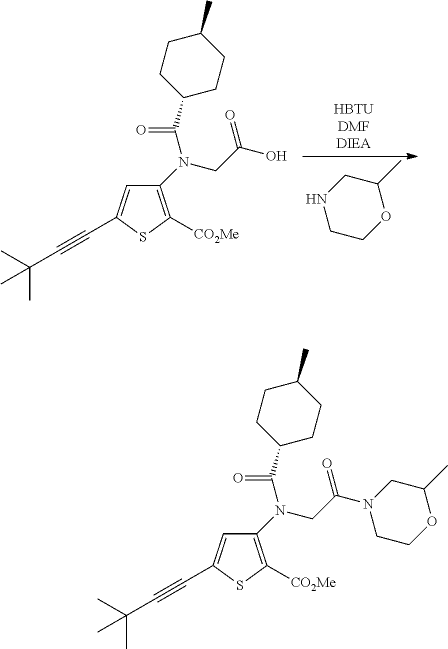

Prepared according to the amide forming procedure of compound 6 to give desired ester product, which was isolated and used as is in the next step without further purification (119 mg, 99%). Analysis by LCMS (60-98 MeOH/H₂O, formic acid modifier, 5/7 minutes, C18); RT=4.25 min, MH+=503.04.

### Step 2

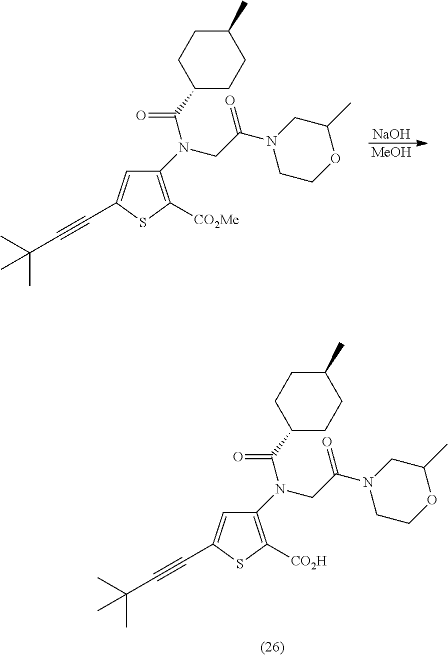

### Compound 26

Prepared according to the hydrolysis procedure of compound 6 to give compound 26, (62 mg, 52%). Analysis by LCMS (40-80% CH₃CN/H₂O, formic acid modifier, 7 min, C18); RT=4.61 min, MH+=489.1.

### Preparation of Compound 35

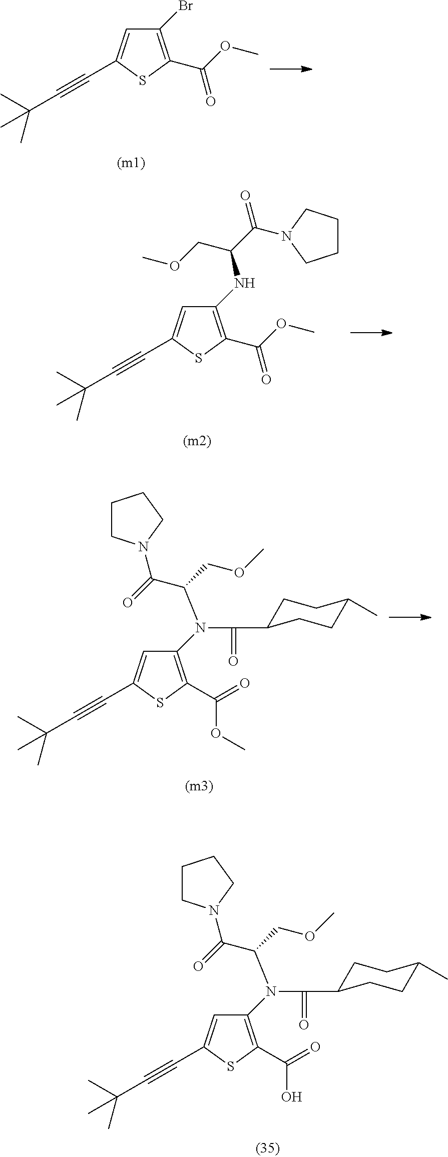

### Compound 35

Prepared as in compound 23. LCMS: 503.35 (MH+). ¹H NMR (300 MHz, DMSO) δ 13.35 (brs, 1H), 7.34 (s, 0.4H), 7.02 (s, 0.6H), 5.66 (t, J=7.0 Hz, 0.4H), 5.17 (t, J=6.8 Hz, 0.6H), 3.77-2.81 (m, 10H), 2.13-0.98 (m, 21H), 0.96-0.43 (m, 4H).

### Preparation of Compound 56

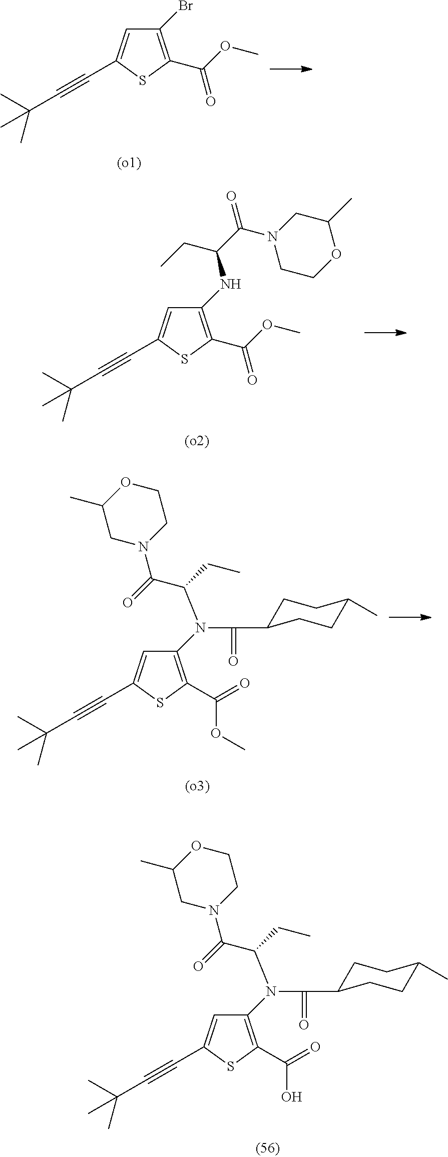

### Compound 56

Prepared as in compound 23. LCMS: 517.36 (MH+). ¹H NMR (300 MHz, DMSO) δ 13.55 (br, 1H), 7.44-6.97 (m, 1H), 5.80-5.06 (m, 1H), 4.36-3.08 (m, 8H), 2.15-0.95 (m, 20H), 0.89-0.45 (m, 9H).

### Preparation of Compound 61

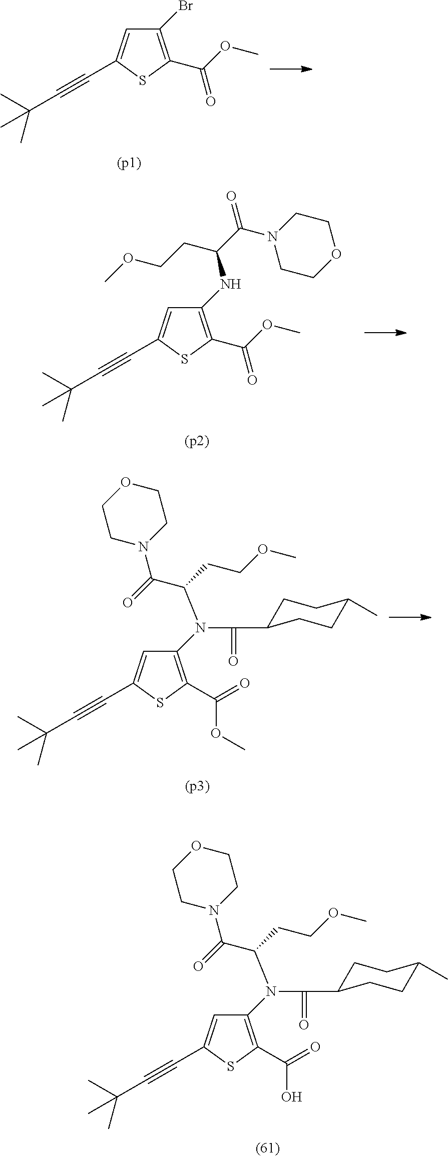

### Compound 61

Prepared as in compound 23. LCMS: 533.36 (MH+). ¹H NMR (300 MHz, CDCl₃) δ 7.50 (s, 0.4H), 6.76 (s, 0.6H), 5.52 (dd, J=9.4, 5.7 Hz, 0.4H), 5.27 (dd, J=10.7, 4.9 Hz, 0.6H), 4.09-3.54 (m, 11H), 3.42-3.18 (m, 3H), 2.14-1.22 (m, 20H), 0.91-0.59 (m, 4H).

### Preparation of Compound 64

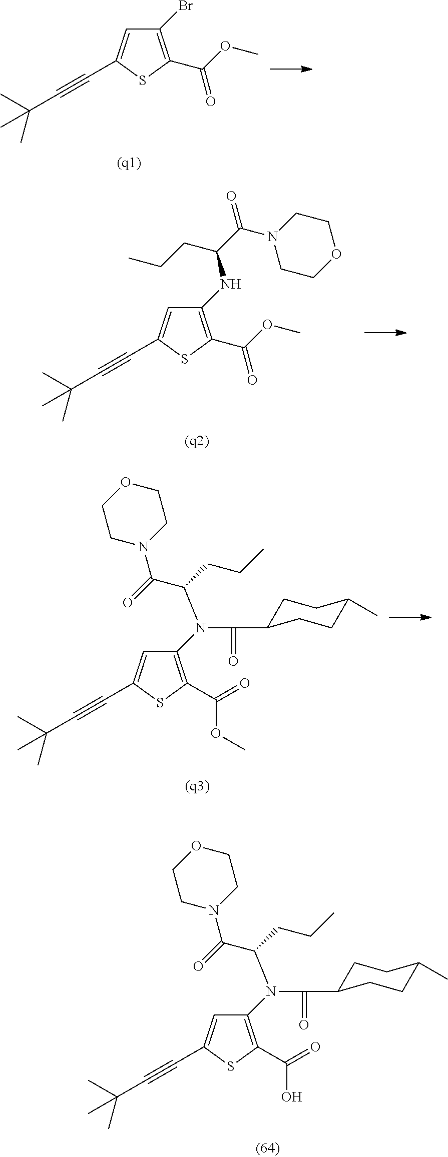

### Compound 64

Prepared as in compound 23. LCMS: 517.43 (MH+). ¹H NMR (300 MHz, CDCl₃) δ 7.43 (s, 0.5H), 6.68 (s, 0.5H), 5.37-5.00 (m, 1H), 3.96-3.33 (m, 9H), 2.05-1.07 (m, 20H), 0.96-0.50 (m, 8H).

### Preparation of Compound 78

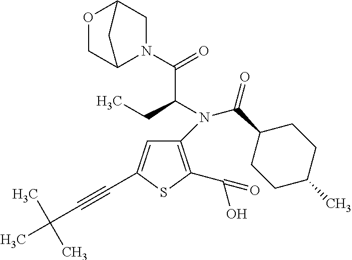 [SMILES: CC[C@@H](C(=O)N1CC2CC1CO2)N(c1cc(C#CC(C)(C)C)sc1C(=O)O)C(=O)[C@H]1CC[C@H](C)CC1]

Compound 78 was prepared as described for compound 82: LC-MS: 515 as M+1 peak at 1.45 min. LC-MS: 515.2 as MH+peak at 3.2 min; NMR (300 MHz, CDCl₃) δ 7.67-7.49 (d, 1H), 6.79-6.67 (d, 1H); 5.23-4.98 (m, 1H), 4.85-4.61 (m, 2H), 4.42 (ddd, J=38.6, 20.9, 6.5 Hz, 1H), 4.11 (dd, J=36.5, 8.5 Hz, 1H), 3.88-3.74 (m, 2H); 3.49 (dd, J=59.9, 11 Hz, 2H); 2.14-1.82 (m, 3H); 1.77-1.39 (m, 7H), 1.39-1.27 (m, 9H); 1.04-0.54 (m, 9H).

### Preparation of Compound 79

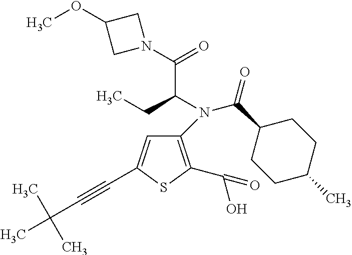 [SMILES: CC[C@@H](C(=O)N1CC(OC)C1)N(c1cc(C#CC(C)(C)C)sc1C(=O)O)C(=O)[C@H]1CC[C@H](C)CC1]

Compound 79 was prepared as described for compound 82: LC-MS: 503 as MH+ peak at 1.68 min.

### Preparation of Compound 83

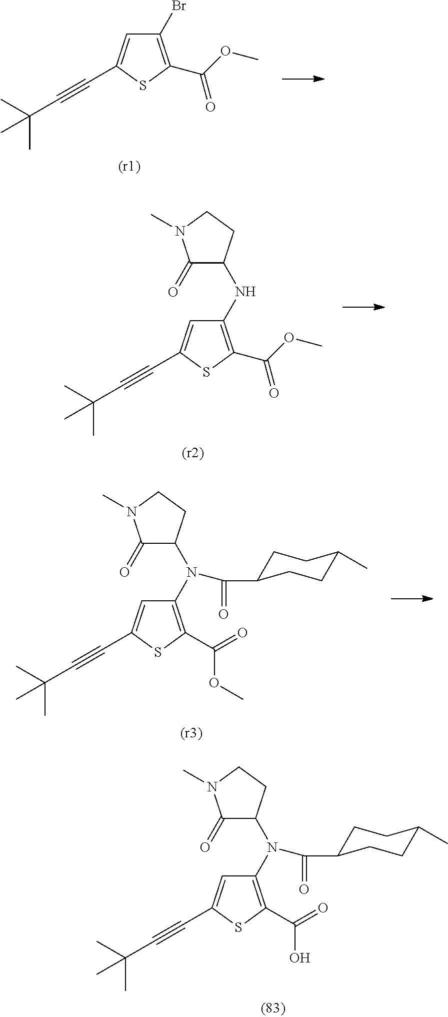

### Compound 83

Prepared as in compound 82. LCMS: 445.0 (MH+). ¹H NMR (300 MHz, CDCl₃) δ 6.94 (s, 0.5H), 6.91 (s, 0.5H), 5.15 (t, J=9.5 Hz, 0.5H), 4.34 (t, J=9.4 Hz, 0.5H), 3.84-3.32 (m, 2H), 2.94 (s, 1.5H), 2.88 (s, 1.5H), 2.60-2.26 (m, 1H), 2.23-1.97 (m, 1H), 1.94-1.16 (m, 17H), 0.95-0.57 (m, 5H).

### Preparation of Compounds 91 and 92

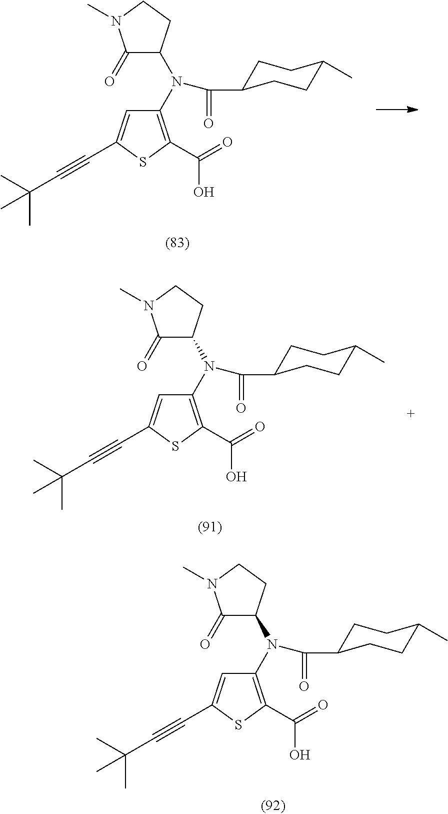

The two enantiomers of Compound 83 were separated using SFC under following conditions: Column: Whelk-O (10×250); Mobile phase: 15% EtOH, 85% CO₂; Flow rate: mL/min; pressure: 100 bar.

### Preparation of Compound 94

Step 1

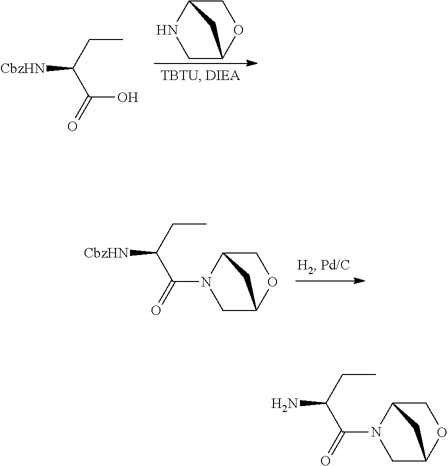 [SMILES: CC[C@H](NC(=O)OCc1ccccc1)C(=O)O ; C1O[C@@H]2CN[C@H]1C2 ; CC[C@H](NC(=O)OCc1ccccc1)C(=O)N1C[C@@H]2C[C@H]1CO2 ; CC[C@H](N)C(=O)N1C[C@@H]2C[C@H]1CO2]

To a solution of (2S)-2-(benzyloxycarbonylamino)butanoic acid (1.2 g, 4.9 mmol), (1S,4S)-6-oxa-3-azabicyclo[2.2.1]heptane hydrochloride (800 mg, 5.9 mmol), TBTU (1.9 g, 5.9 mmol) in DMF (5 mL) was added DIEA (1.9 g, 2.6 mL, 15 mmol). The mixture was stirred for 4 h and then diluted with EtOAc and H₂O. Organic layer was washed with H₂O and brine, dried over Na₂SO₄ and concentrated in vacuum. The crude was purified from column with 20-70% EtOAc/hex. This afforded desired product as colorless oil (1.2 g, 76%). LC-MS: 319 as MH+ peak at 2.34 min.

To a solution of benzyl N-[(1S)-1-[(1S,4S)-2-oxa-5-azabicyclo[2.2.1]heptane-5-carbonyl]propyl]carbamate (1.2 g, 3.8 mmol) in MeOH (20 mL) was added Pd(OH)₂ (120 mg, 0.85 mmol). The mixture was stirred for 2 h under H₂ balloon. The crude was filtered though a layer of Celite. The filtrate was concentrated. The residue was carried to next step. LC-MS: 184 as MH+ peak at 1.0 min.

### Step 2

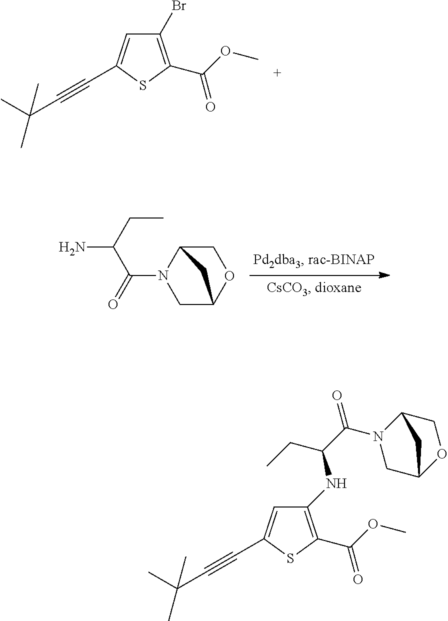 [SMILES: COC(=O)c1sc(C#CC(C)(C)C)cc1Br ; CCC(N)C(=O)N1C[C@@H]2C[C@H]1CO2 ; CC[C@H](Nc1cc(C#CC(C)(C)C)sc1C(=O)OC)C(=O)N1C[C@@H]2C[C@H]1CO2]

A mixture of methyl 3-bromo-5-(3,3-dimethylbut-1-ynyl)thiophene-2-carboxylate (750 mg, 2.5 mmol), (2S)-2-amino-1-[(1S,4S)-2-oxa-5-azabicyclo[2.2.1]heptan-5-yl]butan-1-one (600 mg, 3.26 mmol), Pd₂(dba)₃ (230 mg, 0.25 mmol), rac-BINAP (312 mg, 0.5 mmol) and cesium carbonate (2.4 g, 7.5 mmol) was degassed and filled with N₂. To the mixture was added dioxane (10 mL) and bubbled with N₂ for 20 min. The mixture was stirred for 18 h at 90° C. and diluted with EtOAc. The crude reaction mixture was filtered though a layer of Celite and the filtrate was concentrated. The crude was purified from column 0-60% EtOAc/hex to afford desired product (530 mg, yield 53%) as white solid. LC-MS: 405 as MH+ peak at 3.28 min.

### Step 3

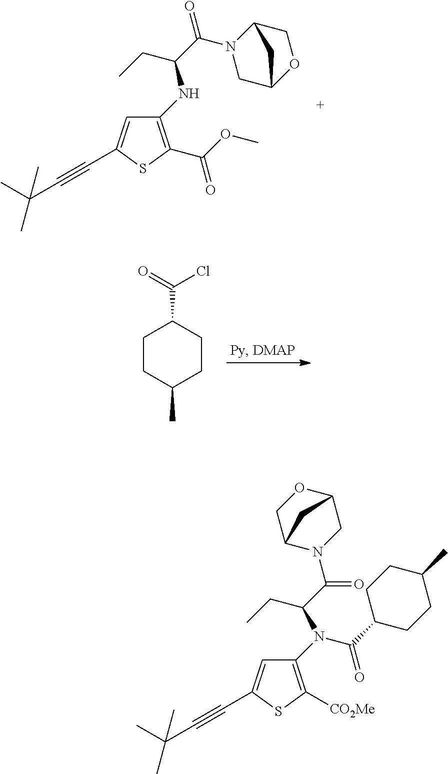

A mixture of methyl 5-(3,3-dimethylbut-1-ynyl)-3-[[(1S)-1-[(1S,4S)-2-oxa-5-azabicyclo[2.2.1]heptane-5-carbonyl]propyl]amino]thiophene-2-carboxylate (500 mg, 1.24 mmol), 4-methylcyclohexanecarbonyl chloride (993 mg, 6.18 mmol), Py (980 mg, 1.0 mL, 12 mmol) and DMAP (151 mg, 1.24 mmol) in DCE (20 mL) was reflux for 24 h. The mixture was diluted with EtOAc, washed with sat. NaHCO₃ solution, dried over Na₂SO₄ and concentrated in vacuum. The crude was purified from column 0-60% EtOAc/hex. This afforded desired product (600 mg, yield 92%). LC-MS: 529.52 as MH+ peak at 4.5 min

### Step 4

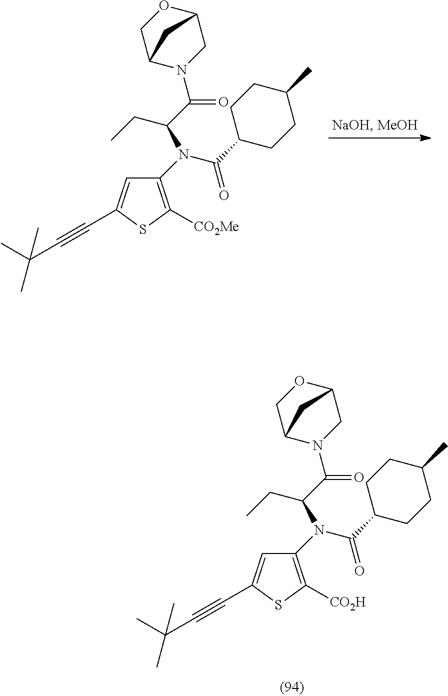

### Compound 94

A solution of methyl 5-(3,3-dimethylbut-1-ynyl)-3-[(trans-4-methylcyclohexanecarbonyl)-[(1S)-1-[(1S,4S)-3-oxa-6-azabicyclo[2.2.1]heptane-6-carbonyl]propyl]amino]thiophene-2-carboxylate (600 mg, 1.14 mmol) in MeOH (10 mL) was added NaOH (5.7 mL of 1 M, 5.67 mmol). The mixture was stirred overnight and then neutralized with HCl to PH=1. The mixture was concentrated in vacuum, extracted with DCM. The organic extracts was dried over Na₂SO₄ and concentrated in vacuo. The crude was purified from column 0-4% MeOH/DCM to afford compound 94 (430 mg, yield 73%). LC-MS: 515.2 as MH+ peak at 3.2 min. NMR (300 MHz, CDCl₃) δ 7.67-7.49 (d, 1H), 6.79-6.67 (d, 1H); 5.23-4.98 (m, 1H), 4.85-4.61 (m, 2H), 4.42 (ddd, J=38.6, 20.9, 6.5 Hz, 1H), 4.11 (dd, J=36.5, 8.5 Hz, 1H), 3.88-3.74 (m, 2H); 3.49 (dd, J=59.9, 11 Hz, 2H); 2.14-1.82 (m, 3H); 1.77-1.39 (m, 7H), 1.39-1.27 (m, 9H); 1.04-0.54 (m, 9H).

### Preparation of Compound 95

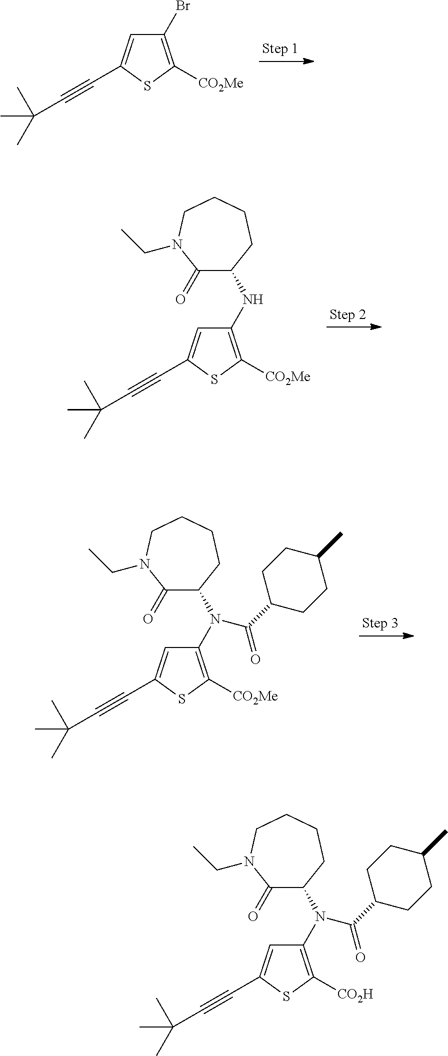

### Step 1

### (S)-methyl 5-(3,3-dimethylbut-1-yn-1-yl)-3-((1-methyl-2-oxoazepan-3-yl)amino)thiophene-2-carboxylate

To a degassed suspension of methyl 3-bromo-5-(3,3-dimethylbut-1-yn-1-yl)thiophene-2-carboxylate (1.4 g, 4.66 mmol), cesium carbonate (3.0 g, 9.33 mmol), and (S)-3-amino-1-methylazepan-2-one HCl (1.0 g, 5.60 mmol) in toluene (14 mL) was added palladium acetate (105 mg, 0.47 mmol) followed by (±)-BINAP (581 mg, 0.93 mmol). The reaction mixture was heated to 90° C. and stirred until the starting material was consumed (monitored by HPLC and LCMS). The reaction mixture was cooled to room temperature, filtered through Celite, and the Celite pad rinsed with dichloromethane. The solvent was removed under reduced pressure and the crude product purified by column chromatography, eluting with 0-90% ethyl acetate/hexanes to afford the desired product, (S)-methyl 5-(3,3-dimethylbut-1-yn-1-yl)-3-((1-methyl-2-oxoazepan-3-yl)amino)thiophene-2-carboxylate (1.1 g, 65%). MS: m/z (obs.) 363.24 [M+H]⁺. ¹H NMR (300 MHz, MeOD) δ 6.60 (s, 1H), 4.45 (d, J=10.8 Hz, 1H), 3.84-3.73 (m, 4H), 3.02 (s, 3H), 2.04-1.75 (m, 4H), 1.60-1.40 (m, 2H), 1.30 (s, 9H). Chiral SFC: 99:1 S/R ratio (Chiralpak IC 30% (40:60:0.2% MeOH/IPA/ESA)/CO₂)

### Step 2

### Methyl 5-(3,3-dimethylbut-1-yn-1-yl)-3-(4-methyl-N—((S)-1-methyl-2-oxoazepan-3-yl)cyclohexanecarboxamido)thiophene-2-carboxylate

(S)-methyl 5-(3,3-dimethylbut-1-yn-1-yl)-3-((1-methyl-2-oxoazepan-3-yl)amino)thiophene-2-carboxylate (1.1 g, 3.04 mmol) was dissolved in DCE (11 mL). Pyridine (245 μL, 3.04 mmol), DMAP (37 mg, 0.30 mmol), and trans-4-methylcyclohexanecarbonyl chloride (1.22 g, 7.59 mmol) were added and the reaction mixture heated to 90° C. until the starting material was consumed (monitored by TLC and HPLC). The reaction mixture was cooled to RT and washed with 2N HCl, sat. NaHCO₃, and brine. The organic layer was dried (Na₂SO₄), filtered and the solvent removed under reduced pressure. The crude product was purified by column chromatography, eluting with 0-90% ethyl acetate/hexanes, to afford the desired product, methyl 5-(3,3-dimethylbut-1-yn-1-yl)-3-(4-methyl-N—((S)-1-methyl-2-oxoazepan-3-yl)cyclohexanecarboxamido)thiophene-2-carboxylate (1.35 g, 91%). MS: m/z (obs.) 487.28 [M+H]⁺. 1H NMR (300 MHz, MeOD) δ 7.47 (s, 1H), 5.29 (d, J=11.7 Hz, 1H), 3.83 (s, 4H), 3.01 (s, 3H), 2.14-1.98 (m, 1H), 1.9-1.4 (m, 10H), 1.37-1.19 (m, 12H), 1.06 (ddd, J=15.6, 12.7, 3.4 Hz, 1H), 0.81 (d, J=6.5 Hz, 3H), 0.76-0.55 (m, 2H).

### Step 3

### 5-(3,3-dimethylbut-1-yn-1-yl)-3-(4-methyl-N—((S)-1-methyl-2-oxoazepan-3-yl)cyclohexanecarboxamido)thiophene-2-carboxylic acid

Methyl 5-(3,3-dimethylbut-1-yn-1-yl)-3-(4-methyl-N—((S)-1-methyl-2-oxoazepan-3-yl)cyclohexanecarboxamido)thiophene-2-carboxylate (1.3 g, 2.67 mmol) was dissolved in THF (6.5 mL) and water (6.5 mL) and lithium hydroxide (192 mg, 8.01 mmol) added. The reaction mixture was stirred at room temperature until the starting material was consumed (monitored by TLC). The reaction mixture was acidified with 3N HCl and washed with ethyl acetate and brine. The organic layer was dried (Na₂SO₄), filtered, and the solvent removed under reduced pressure to give the crude product as a pale yellow foam which was crystallized from acetonitrile to afford compound 95 (1.1 g, 87%). MS: m/z (obs.) 473.26 [M+H]⁺, 471.47 [M−H]⁻. ¹H NMR (300 MHz, MeOD) δ 7.45 (s, 1H), 5.28 (d, J=10.8 Hz, 1H), 3.78 (dd, J=15.4, 11.3 Hz, 1H), 3.01 (s, 3H), 2.19-2.02 (m, 1H), 1.97-1.38 (m, 10H), 1.33 (s, 9H), 1.27-1.19 (m, 1H), 1.17-0.98 (m, 1H), 0.81 (d, J=6.5 Hz, 3H), 0.67 (ddd, J=21.7, 14.4, 10.6 Hz, 2H). Chiral HPLC: 99:1 S/R (Chiralpak IC, 100% ACN/0.1% TFA).

### Preparation of Compound 143

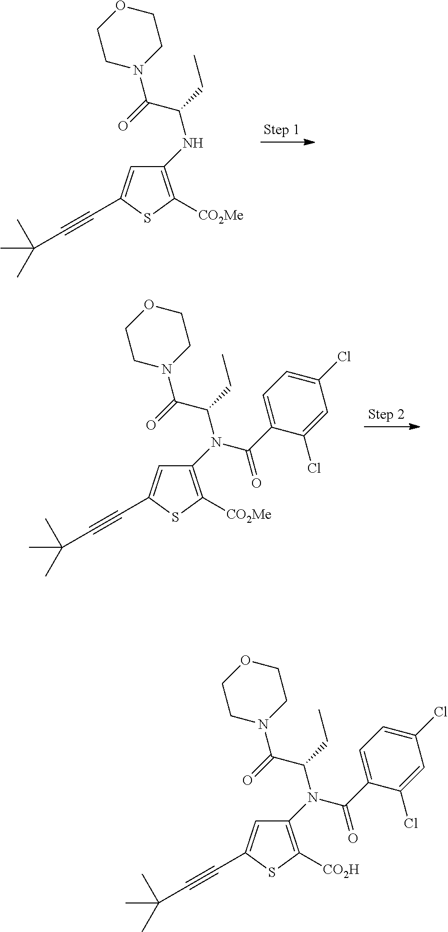 [SMILES: CC[C@H](Nc1cc(C#CC(C)(C)C)sc1C(=O)OC)C(=O)N1CCOCC1 ; CC[C@@H](C(=O)N1CCOCC1)N(C(=O)c1ccc(Cl)cc1Cl)c1cc(C#CC(C)(C)C)sc1C(=O)OC ; CC[C@@H](C(=O)N1CCOCC1)N(C(=O)c1ccc(Cl)cc1Cl)c1cc(C#CC(C)(C)C)sc1C(=O)O]

### Step 1

### (S)-Methyl 5-(3,3-dimethylbut-1-ynyl)-3-[(2,4-dichlorobenzoyl)-[1-(morpholine-4-carbonyl)propyl]amino]-thiophene-2-carboxylate

A solution of (S)-methyl 5-(3,3-dimethylbut-1-ynyl)-3-((1-morpholino-1-oxobutan-2-yl)amino)thiophene-2-carboxylate (450 mg, 1.15 mmol), 2,4-dichlorobenzoyl chloride (1.20 g, 800 μL, 5.73 mmol), and pyridine (185 μL, 2.29 mmol) in 1,2-dichloroethane (9 mL) was heated in oil bath at 90° C. for 14 hrs, then diluted with MeOH and evaporated. The residue was purified by silica gel chromatography eluting with a gradient of 1% to 70% EtOAc in hexane over 35 min. Yield 620 mg, 96%. MS: m/z (obs.) 565.2 [M+H]⁺.

### Step 2

### (S)-5-(3,3-Dimethylbut-1-ynyl)-3-[(2,4-dichlorobenzoyl)-[1-(morpholine-4-carbonyl)propyl]amino]-thiophene-2-carboxylic acid

To a solution of (S)-methyl 5-(3,3-dimethylbut-1-ynyl)-3-[(2,4-dichlorobenzoyl)-[1-(morpholine-4-carbonyl)propyl]amino]-thiophene-2-carboxylate (620 mg, 1.1 mmol) in THF (10 mL) was added a solution of LiOH (263 mg, 11 mmol) in water (10 mL). The mixture was stirred at ambient temperature for 15 hrs, acidified with 6N HCl to pH=1, and the THF solvent evaporated. The resulting acidic solution was cooled to 0-10° C., and the gummy precipitate was collected via filtration, washed with water and dried to afford solid 143. Yield: 556 mg, 87%. MS: m/z (obs.) 551.1 [M+H]⁺. 1H NMR (400 MHz, CDCl₃) δ 7.68 (s, 0.5H), 7.29 (m, 2H), 7.22-7.04 (m, 1H), 6.96 (s, 0.5H), 5.49 (m, 1H), 4.26-3.42 (m, 8H), 1.96-1.45 (m, 2H), 1.31 (s, 9H), 0.96 (m, 3H).

### Preparation of Compound 140

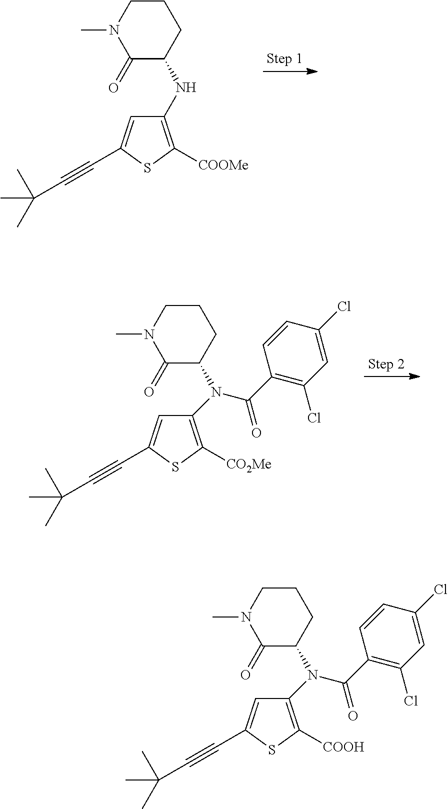 [SMILES: COC(=O)c1sc(C#CC(C)(C)C)cc1N[C@H]1CCCN(C)C1=O ; COC(=O)c1sc(C#CC(C)(C)C)cc1N(C(=O)c1ccc(Cl)cc1Cl)[C@H]1CCCN(C)C1=O ; CN1CCC[C@H](N(C(=O)c2ccc(Cl)cc2Cl)c2cc(C#CC(C)(C)C)sc2C(=O)O)C1=O]

### Step 1

### (S)-Methyl 5-(3,3-dimethylbut-1-ynyl)-3-[(2,4-dichlorobenzoyl)-N-(1-methyl-2-oxopiperidin-3-yl)amino]-thiophene-2-carboxylate

(S)-Methyl 5-(3,3-dimethylbut-1-ynyl)-3-[N-(1-methyl-2-oxopiperidin-3-yl)amino]thiophene-2-carboxylate (470 mg, 1.349 mmol), pyridine (196 μL, 2.43 mmol), DMAP (16.5 mg, 0.13 mmol), and 2,4-dichlorobenzoyl chloride (380 μL, 2.7 mmol) were dissolved in DCE (4.7 mL) and the mixture heated to 90° C. overnight. The reaction was cooled to and quenched by addition of 2N HCl to pH3. The mixture was extracted with CH₂Cl₂ (2×5 vol) and the combined organics were washed with saturated aqueous NaHCO₃ (10 vol) and brine, and dried (MgSO4). The solution was evaporated and the residue purified by silica gel chromatography (40 g ISCO column, eluted with a gradient of 5% to 40% EtOAc in heptane over 4 column volumes; hold at 40% for 3 column volumes and continued gradient from 40%-90% EtOAc over 5 column volumes). Yield: 614 mg, 87.29%. MS: m/z (obs.) 521.1 [M+H]⁺.

### Step 2

### (S)-5-(3,3-Dimethylbut-1-ynyl)-3-[(2,4-dichlorobenzoyl)-N-(1-methyl-2-oxopiperidin-3-yl)amino]-thiophene-2-carboxylic acid

(S)-Methyl 5-(3,3-dimethylbut-1-ynyl)-3-[(2,4-dichlorobenzoyl)-N-(1-methyl-2-oxopiperidin-3-yl)amino]-thiophene-2-carboxylate (614 mg, 1.18 mmol) was dissolved in THF (3 mL) and water (3 mL) and LiOH (113 mg, 4.71 mmol) was added. The mixture was stirred at ambient temperature overnight. pH was adjusted to 7 (1N NaOH) and the solution extracted with EtOAc (2×6 vol). The combined extracts were washed with water, brine (8 vol. each) and evaporated to dryness. The residue was taken into MeCN and water and lyophilized to give 140. MS: m/z (obs.) 507.0 [M+H]⁺. 1H NMR (300 MHz, d₆-DMSO) δ 7.46 (d, J=8.2 Hz, 1H), 7.39 (s, 1H), 7.27 (d, 1H), 7.15 (s, 1H), 4.65 (s, 1H), 3.28 (s, 2H), 2.90 (s, 3H), 2.35 (s, 1H), 2.20-2.03 (m, 1H), 1.88 (s, 2H), 1.30 (d, J=3.5 Hz, 9H).

### Preparation of Compound 148

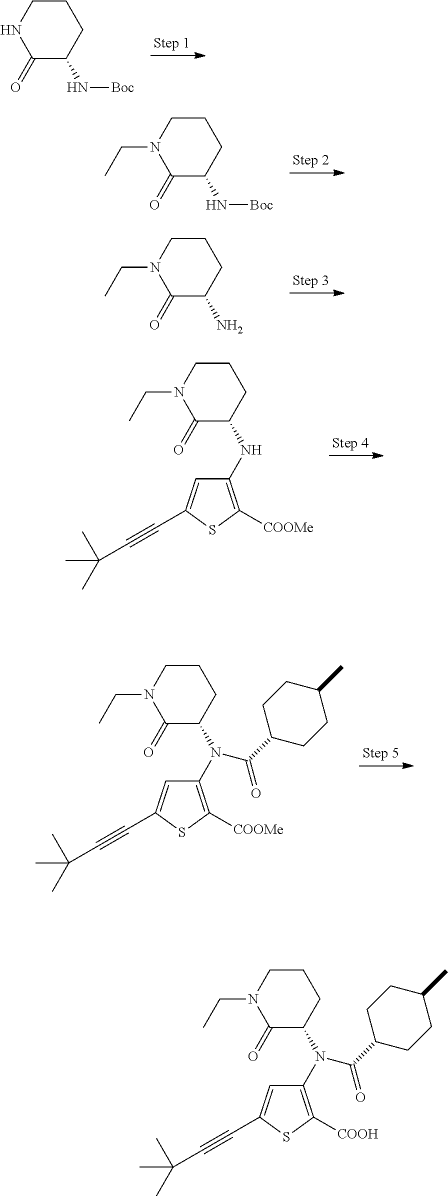

### Step 1

### (S)-tert-Butyl (1-ethyl-2-oxopiperidin-3-yl)carbamate

(S)-tert-butyl (2-oxopiperidin-3-yl)carbamate (1 g, 4.67 mmol) was dissolved in THF (10 mL) and the solution cooled to 0° C. A 1M solution of LiHMDS (5.4 mL) in THF was added and the mixture stirred for 1 h. Iodoethane (410 μL, 5.1 mmol) was added and the reaction removed from the cooling bath and allowed to warm to room temperature and stirred overnight. The reaction was quenched with saturated aqueous NH₄Cl (8 vol) and extracted with EtOAc (2×4 vol). The combined extracts were washed with brine, dried, filtered and evaporated. The desired product was isolated by silica gel chromatography to afford tert-butyl N-[(3S)-1-ethyl-2-oxo-3-piperidyl]carbamate. Yield: (190 mg, 16.8%). MS: m/z (obs.) 507.0 [M+H]⁺. 1H NMR (400 MHz, CDCl₃) δ 6.86 (d, J=7.8 Hz, 1H), 3.85 (s, 1H), 3.30-3.15 (m, 5H), 1.84 (ddd, J=26.1, 16.9, 12.3 Hz, 5H), 1.38 (s, 10H), 1.01 (t, J=7.1 Hz, 3H).

### Step 2

### (S)-3-Amino-1-ethylpiperidin-2-one hydrochloride

Tert-Butyl N-[(3S)-1-ethyl-2-oxo-3-piperidyl]carbamate was dissolved in diethyl ether (1.9 mL) and a solution of 4N HCl in dioxane (0.4 mL) was added and the mixture stirred overnight at room temperature. Addition 4 N HCl in dioxane was added and the reaction stirred for 5 h. The solvent was evaporated and the product dissolved in MeCN and H₂O and lyophilized. The product was used in the next step without further purification or characterization.

### Step 3

### (S)-Methyl 5-(3,3-dimethylbut-1-ynyl)-3-[N-(1-ethyl-2-oxopiperidin-3-yl)amino]thiophene-2-carboxylate

(S)-3-Amino-1-ethyl-piperidin-2-one hydrochloride (140 mg, 0.78 mmol) and cesium carbonate (638 mg, 1.96 mmol) were dissolved in 1,4-dioxane (4 mL). Methyl 3-bromo-5-(3,3-dimethylbut-1-ynyl)thiophene-2-carboxylate (197 mg, 0.65 mmol) was added and the mixture was degassed for 2 hour by N₂ purge. Pd(OAc)₂ (14.7 mg, 0.065 mmol) and rac-BINAP (81.3 mg, 0.13 mmol) were added and the mixture further purged with N2 for 30 min. then heated to 100° C. overnight. After cooling to RT the mixture was diluted with EtOAc (10 vol), filtered through Celite and evaporated to dryness. The product was purified by silica gel chromatography (120 g ISCO column, eluted with a gradient of 10% to 50% EtOAc in heptane over 4 column volumes; hold at 50% for 3 column volumes and continued gradient from 50%-90% EtOAc over 7 column volumes). The desired product eluted at about 10 CV. Yield 100 mg, 42.3%). MS: m/z (obs.) 363.4 [M+H]⁺.

### Step 4

### Methyl(S)-5-(3,3-dimethylbut-1-ynyl)-3-((trans)-4-methyl-N-(1-ethyl-2-oxopiperidin-3-yl)cyclohexanecarboxamido)thiophene-2-carboxylate

Acylation of (S)-methyl 5-(3,3-dimethylbut-1-ynyl)-3-[N-(1-ethyl-2-oxopiperidin-3-yl)amino]thiophene-2-carboxylate was performed as described for other examples. Yield 58 mg, 43%. MS: m/z (obs.) 487.2 [M+H]⁺.

### Step 5

### (S)-5-(3,3-Dimethylbut-1-ynyl)-3-((trans)-4-methyl-N-(1-ethyl-2-oxopiperidin-3-yl)cyclohexanecarboxamido)thiophene-2-carboxylic acid

Hydrolysis of methyl(S)-5-(3,3-dimethylbut-1-ynyl)-3-((trans)-4-methyl-N-(1-ethyl-2-oxopiperidin-3-yl)cyclohexanecarboxamido)thiophene-2-carboxylate (58 mg), was carried out as described for other examples to give 148. Yield 54 mg, 93%. MS: m/z (obs.) 473.2 [M+H]⁺. 1H NMR (400 MHz, CDCl₃) δ 6.76 (s, 1H), 3.68-3.58 (m, 1H), 3.51 (m, 1H), 3.41 (m, 2H), 3.18 (m, 1H), 2.37 (dd, J=23.5, 12.2 Hz, 1H), 2.08 (m, 2H), 1.95 (d, J=13.6 Hz, 1H), 1.73 (m, 2H), 1.62-1.41 (m, 4H), 1.26 (s, 9H), 1.12 (m, 5H), 0.73 (d, J=6.5 Hz, 3H), 0.69-0.57 (m, 2H).

### Preparation of Compound 149

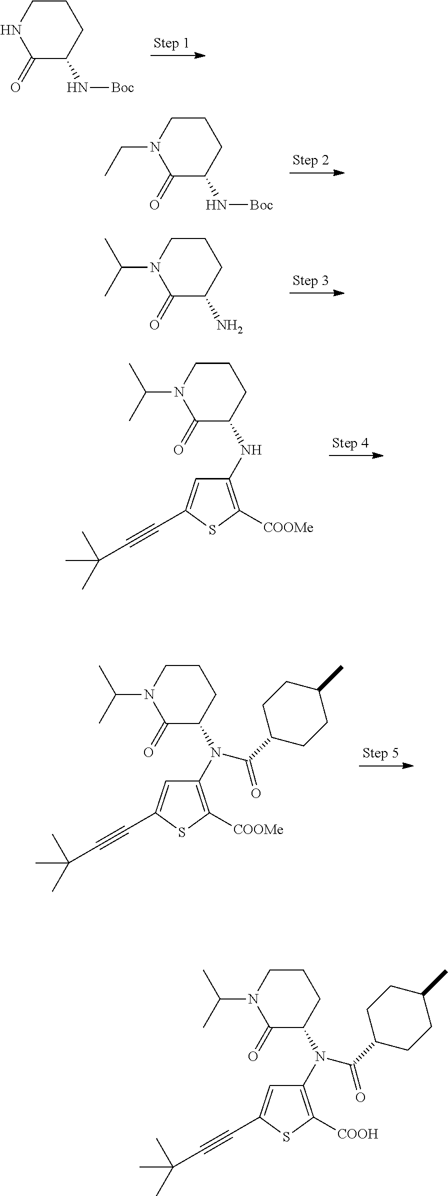

### Step 1

### (S)-tert-Butyl (1-isopropyl-2-oxopiperidin-3-yl)carbamate

(S)-tert-butyl (2-oxopiperidin-3-yl)carbamate (1.21 g, 5.69 mmol) was dissolved in DMSO (12 mL). KOH (415 mg, 7.39 mmol) was added, followed by 2-iodopropane (740 μL, 7.4 mmol) and the mixture stirred for 72 h. The reaction was quenched by addition of saturated aqueous NH₄Cl (8 vol) and extracted with EtOAc (2×4 vol). The combined extracts were washed with brine, dried, filtered and evaporated. The desired product was isolated by silica gel chromatography to afford tert-butyl N-[(3S)-1-isopropyl-2-oxo-3-piperidyl]carbamate. Yield: (430 mg, 29.5%). MS: m/z (obs.) 279.1 [M+Na]⁺. 1H NMR (400 MHz, d6-DMSO) δ 6.83 (d, J=8.1 Hz, 1H), 4.68-4.52 (m, 1H), 3.88 (d, J=6.8 Hz, 1H), 3.12 (dd, J=13.0, 7.2 Hz, 2H), 1.98-1.85 (m, 1H), 1.75 (dd, J=12.3, 6.8 Hz, 2H), 1.56 (d, J=17.8 Hz, 1H), 1.38 (s, 11H), 1.03 (dd, J=6.7, 2.0 Hz, 7H).

### Step 2

### (S)-3-Amino-1-isopropylpiperidin-2-one hydrochloride

Tert-Butyl N-[(3S)-1-isopropyl-2-oxo-3-piperidyl]carbamate was deprotected as described for Compound 148 and used without purification.

### Step 3

### (S)-Methyl 5-(3,3-dimethylbut-1-ynyl)-3-[N-(1-isopropyl-2-oxopiperidin-3-yl)amino]thiophene-2-carboxylate

(S)-3-Amino-1-isopropyl-piperidin-2-one hydrochloride (323 mg, 1.68 mmol) was reacted with methyl 3-bromo-5-(3,3-dimethylbut-1-ynyl)thiophene-2-carboxylate (421 mg, 1.40 mmol) in the presence of cesium carbonate (1.37 g, 4.2 mmol), Pd(OAc)₂ (31 mg, 0.14 mmol) and rac-BINAP (174 mg, 0.28 mmol) in 1,4-dioxane (4 mL) as described for Compound 148. Yield 314 mg, 59%). MS: m/z (obs.) 399.2 [M+Na]⁺.

### Step 4

### Methyl(S)-5-(3,3-dimethylbut-1-ynyl)-3-((trans)-4-methyl-N-(1-isopropyl-2-oxopiperidin-3-yl)cyclohexanecarboxamido)thiophene-2-carboxylate

Acylation of (S)-methyl 5-(3,3-dimethylbut-1-ynyl)-3-[N-(1-isopropyl-2-oxopiperidin-3-yl)amino]thiophene-2-carboxylate (314 mg) was performed as described for other examples. The product was purified by silica gel chromatography (80 g ISCO column, eluted with a gradient of 5% to 40% EtOAc in heptane over 4 column volumes; hold at 40% for 3 column volumes and continued gradient from 40%-90% EtOAc over 5 column volumes). MS: m/z (obs.) 501.2 [M+H]⁺.

### Step 5

### (S)-5-(3,3-Dimethylbut-1-ynyl)-3-((trans)-4-methyl-N-(1-isopropyl-2-oxopiperidin-3-yl)cyclohexanecarboxamido)thiophene-2-carboxylic acid

Hydrolysis of methyl(S)-5-(3,3-dimethylbut-1-ynyl)-3-((trans)-4-methyl-N-(1-isopropyl-2-oxopiperidin-3-yl)cyclohexanecarboxamido)thiophene-2-carboxylate (400 mg. 0.80 mmol), was carried out as described for other examples. The product was isolated following lyophilization from MeCN/H₂O to give 149. Yield 279 mg, 69%. MS: m/z (obs.) 487.2 [M+H]⁺. 1H NMR (400 MHz, CDCl₃) δ 6.63 (s, 1H), 4.78-4.64 (m, 1H), 3.66-3.47 (m, 1H), 3.21-3.05 (m, 2H), 2.16 (m, 1H), 2.02-1.80 (m, 4H), 1.61 (m, 1H), 1.36 (m, 3H), 1.16-1.08 (s, 9H), 1.09-0.99 (m, 6H), 0.96-0.90 (m, 3H), 0.62 (d, J=6.5 Hz, 3H), 0.54 (m, 2H).

### Preparation of Compound 150

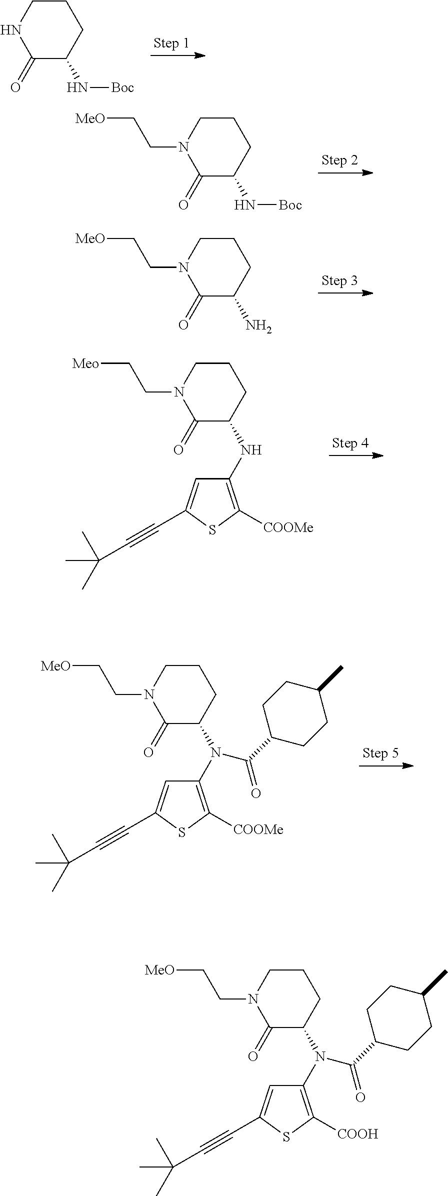

### Step 1

### (S)-tert-Butyl (1-(2-methoxyethyl)-2-oxopiperidin-3-yl)carbamate

(S)-tert-Butyl (2-oxopiperidin-3-yl)carbamate (1.16 g, 5.42 mmol) was dissolved in DMSO (12 mL). KOH (395 mg, 7.05 mmol) was added, followed by 1-bromo-2-methoxyethane (660 μL, 7.0 mmol) and the mixture stirred overnight. The reaction was quenched by addition of saturated aqueous NH₄Cl (8 vol) and extracted with EtOAc (2×4 vol). The combined extracts were washed with brine, dried, filtered and evaporated. The desired product was isolated by silica gel chromatography to afford (S)-tert-butyl (1-(2-methoxyethyl)-2-oxopiperidin-3-yl)carbamate. Yield: (1.04 g, 70.4%).

### Step 2

### (S)-3-Amino-1-(2-methoxyethyl)-piperidin-2-one hydrochloride

(S)-tert-Butyl (1-(2-methoxyethyl)-2-oxopiperidin-3-yl)carbamate was dissolved in isopropyl alcohol) 10 mL) and a solution of 4N HCl in dioxane (5 mL) was added. The mixture was stirred at ambient temperature for 16 h, then evaporated to dryness. The residue was re-dissolved in MeCN/H2O and lyophilized, and used without further purification or characterization. Yield 767 mg, 100%.

### Step 3

### (S)-Methyl 5-(3,3-dimethylbut-1-ynyl)-3-[N-(1-(2-methoxyethyl)-2-oxopiperidin-3-yl)amino]thiophene-2-carboxylate

(S)-3-Amino-1-(2-methoxyethyl)-piperidin-2-one hydrochloride (796 mg, 3.81 mmol) was reacted with methyl 3-bromo-5-(3,3-dimethylbut-1-ynyl)thiophene-2-carboxylate (957 mg, 3.18 mmol) in the presence of cesium carbonate (3.11 g, 9.5 mmol), Pd(OAc)₂ (71 mg, 0.32 mmol) and rac-BINAP (395 mg, 0.64 mmol) in 1,4-dioxane (20 mL) as described for Compound 148.

Yield 430 mg, 34.5%;

MS: m/z (obs.) 393.2 [M+H]⁺;

### Step 4

### Methyl(S)-5-(3,3-dimethylbut-1-ynyl)-3-((trans)-4-methyl-N-(1-(2-methoxyethyl)-2-oxopiperidin-3-yl)cyclohexanecarboxamido)thiophene-2-carboxylate

Acylation of (S)-methyl 5-(3,3-dimethylbut-1-ynyl)-3-[N-(1-(2-methoxyethyl)-2-oxopiperidin-3-yl)amino]thiophene-2-carboxylate (430 mg) was performed as described for other examples. MS: m/z (obs.) 517.2 [M+H]⁺;

### Step 5

### (S)-5-(3,3-Dimethylbut-1-ynyl)-3-((trans)-4-methyl-N-(1-(2-methoxyethyl)-2-oxopiperidin-3-yl)cyclohexanecarboxamido)thiophene-2-carboxylic acid

Hydrolysis of methyl(S)-5-(3,3-dimethylbut-1-ynyl)-3-((trans)-4-methyl-N-(1-(2-methoxyethyl)-2-oxopiperidin-3-yl)cyclohexanecarboxamido)thiophene-2-carboxylate (400 mg. 0.80 mmol), was carried out as described for other examples. Desired 150 was isolated by lyophilization from MeCN/H₂O. Yield 158 mg, 27.8%. MS: m/z (obs.) 503.1 [M+H]⁺. 1H NMR (400 MHz, CDCl₃) δ 6.58 (m, 1H), 3.53-3.39 (m, 2H), 3.35 (m, 2H), 3.26 (m, 3H), 3.12 (s, 3H), 2.23 (m, 1H), 1.91 (m, 2H), 1.76 (m, 1H), 1.55 (m, 1H), 1.46-1.24 (m, 4H), 1.10 (s, 6H), 0.98 (s, 5H), 0.57 (d, J=6.4 Hz, 3H), 0.51 (m, 3H).

### Preparation of Compound 134

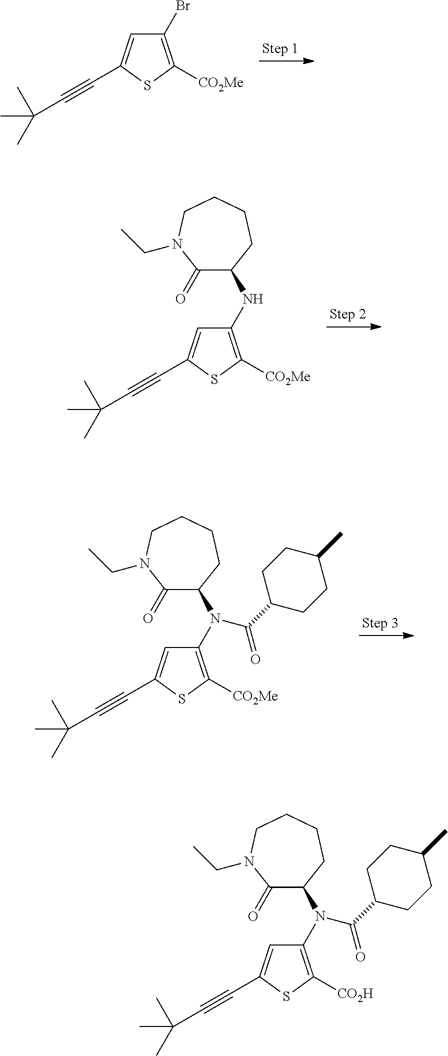

### Step 1

### (S)-Methyl 5-(3,3-dimethylbut-1-ynyl)-3-[N-(1-ethyl-2-oxoazepan-3-yl)amino]thiophene-2-carboxylate

(S)-3-Amino-1-ethylazepan-2-one hydrochloride (384 mg, 1.99 mmol) and cesium carbonate (1.62 g, 5 mmol) were dissolved in 1,4-dioxane (8 mL). Methyl 3-bromo-5-(3,3-dimethylbut-1-ynyl)thiophene-2-carboxylate (500 mg, 1.66 mmol) was added and the mixture was degassed for 2 hour by N₂ purge. Pd(OAc)₂ (37 mg, 0.17 mmol) and rac-BINAP (207 mg, 0.33 mmol) were added and the mixture further purged with N₂ for 30 min. then heated to 100° C. overnight. After cooling to RT the mixture was diluted with EtOAc (10 vol), filtered through Celite and evaporated to dryness. The product was purified by silica gel chromatography (gradient of 0% to 90% EtOAc in heptane). Yield 530 mg, 96%). MS: m/z (obs.) 377.6 [M+H]⁺. 1H NMR (300 MHz, CDCl₃) δ 7.85 (d, 1H), 6.43 (s, 1H), 3.84 (s, 3H), 3.52 (dd, 4H), 1.86 (d, 6H), 1.33 (s, 8H), 1.16 (t, 3H).

### Step 2

### Methyl(S)-5-(3,3-dimethylbut-1-ynyl)-3-((trans)-4-methyl-N-(1-ethyl-2-oxoazepan-3-yl)cyclohexanecarboxamido)thiophene-2-carboxylate

Acylation of (S)-Methyl 5-(3,3-dimethylbut-1-ynyl)-3-[N-(1-ethyl-2-oxoazepan-3-yl)amino]thiophene-2-carboxylate (300 mg) was performed as described for other examples. Yield 250 mg. MS: m/z (obs.) 501.7 [M+H]⁺.

### Step 3

### (S)-5-(3,3-Dimethylbut-1-ynyl)-3-((trans)-4-methyl-N-(1-ethyl-2-oxoazepan-3-yl)cyclohexanecarboxamido)thiophene-2-carboxylic acid

Methyl(S)-5-(3,3-dimethylbut-1-ynyl)-3-((trans)-4-methyl-N-(1-ethyl-2-oxoazepan-3-yl)cyclohexanecarboxamido)thiophene-2-carboxylate (150 mg. 0.30 mmol), was dissolved in a mixture of THF (15 mL) and H2O (3 mL)/LiOH (14 mg, 0.6 mmol was added and the reaction stirred at room temperature overnight. The solution was concentrated to remove THF, then diluted with EtOAc and H₂O. The aqueous phase was separated and acidified with 1N HCl, then extracted with EtOAc. The extract was dried and the product 134 isolated by precipitation from MeCN with diethyl ether. Yield 100 mg, 62%. MS: m/z (obs.) 487.7 [M+H]⁺. 1H NMR (300 MHz, CDCl₃) δ 6.74 (s, 1H), 5.07 (dd, 1H), 3.81 (dd, 1H), 3.71-3.20 (m, 6H), 2.19-1.37 (m, 7H), 1.34 (d, J=3.9 Hz, 9H), 1.17 (q, J=7.2 Hz, 4H), 0.85-0.62 (m, 5H).

### Preparation of Compound 135

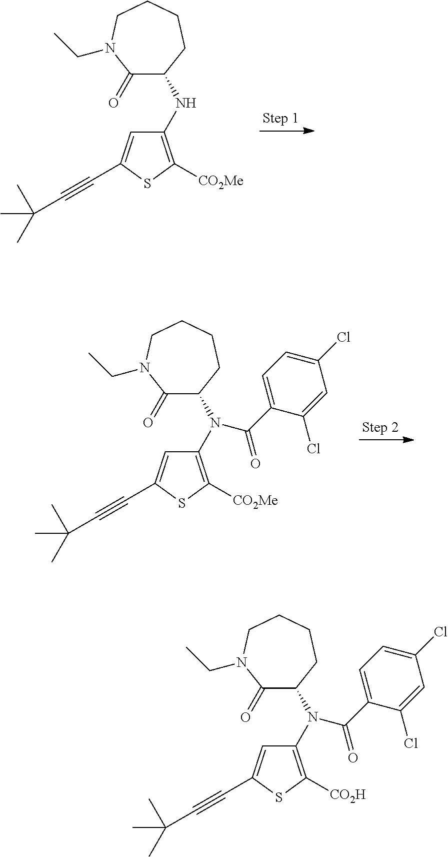 [SMILES: CCN1CCCC[C@H](Nc2cc(C#CC(C)(C)C)sc2C(=O)OC)C1=O ; CCN1CCCC[C@H](N(C(=O)c2ccc(Cl)cc2Cl)c2cc(C#CC(C)(C)C)sc2C(=O)OC)C1=O ; CCN1CCCC[C@H](N(C(=O)c2ccc(Cl)cc2Cl)c2cc(C#CC(C)(C)C)sc2C(=O)O)C1=O]

### Step 1

### (S)-Methyl 5-(3,3-dimethylbut-1-ynyl)-3-[(2,4-dichlorobenzoyl)-N-(1-ethyl-2-oxoazepan-3-yl)amino]-thiophene-2-carboxylate

(S)-Methyl 5-(3,3-dimethylbut-1-ynyl)-3-[N-(1-ethyl-2-oxoazepan-3-yl)amino]thiophene-2-carboxylate (200 mg, 0.50 mmol), pyridine (196 μL, 2.43 mmol), DMAP (6 mg, 0.05 mmol), and 2,4-dichlorobenzoyl chloride (1.0 mmol) were dissolved in DCE (14 mL) and the mixture heated to 90° C. overnight. The reaction was cooled to and diluted with 2N HCl to pH3. The organic phase was washed with brine, and dried (MgSO₄). The solution was evaporated and the residue purified by silica gel chromatography. Yield: 150 mg. MS: m/z (obs.) 549.6 [M+H]⁺.

### Step 2

### (S)-5-(3,3-dimethylbut-1-ynyl)-3-[(2,4-dichlorobenzoyl)-N-(1-ethyl-2-oxoazepan-3-yl)amino]-thiophene-2-carboxylic acid

(S)-Methyl 5-(3,3-dimethylbut-1-ynyl)-3-[(2,4-dichlorobenzoyl)-N-(1-ethyl-2-oxoazepan-3-yl)amino]-thiophene-2-carboxylate (30 mg, 0.06 mmol) was hydrolyzed as described for Compound 134 to give desired 135. Yield 25 mg, 90%. MS: m/z (obs.) 535.5 [M+H]⁺. 1H NMR (300 MHz, CDCl₃) δ 7.42 (d, J=8.3 Hz, 1H), 7.25-7.17 (m, 1H), 6.94 (s), 5.39 (d, J=11.4 Hz, 1H), 3.93-3.73 (m, 1H), 3.64-3.45 (m, 2H), 3.38 (d, 1H), 2.22-1.38 (m, 6H), 1.30 (d, 6H), 1.23-1.11 (m, 2H).

### Preparation of Compound 136

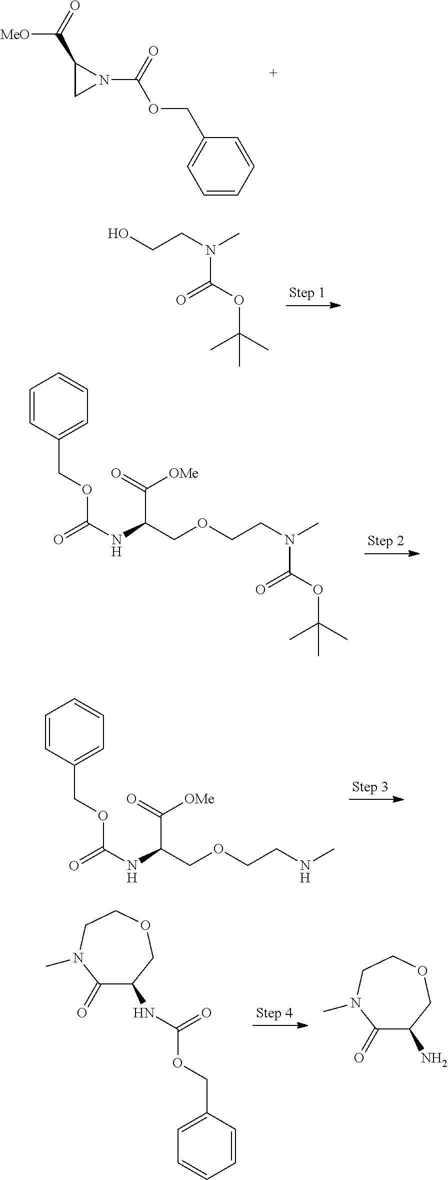

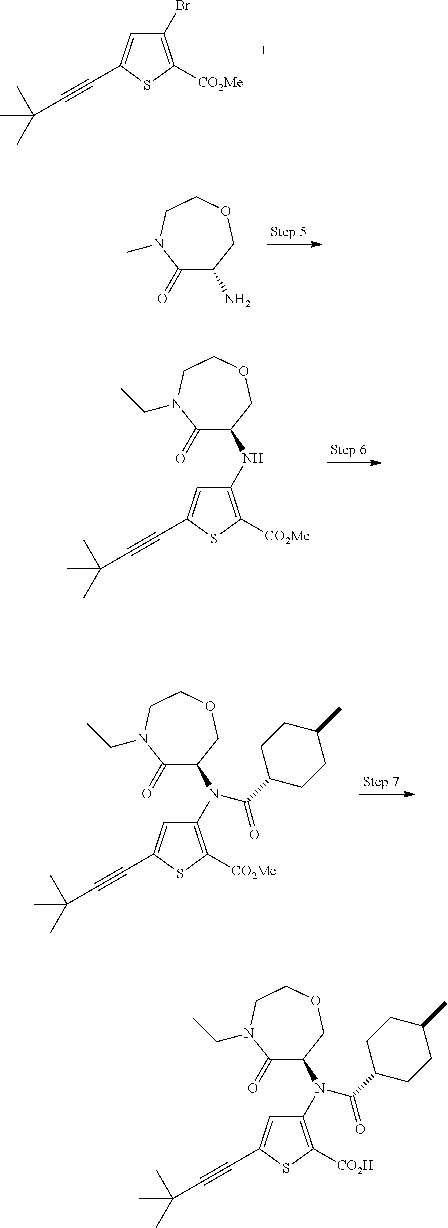

### Step 1

### (S)-Methyl 2-(((benzyloxy)carbonyl)amino)-3-(2-((tert-butoxycarbonyl)-(methyl)amino)ethoxy)propanoate

Boron trifluoride diethyl etherate (1.26 g, 8.95 mmol) was added to a stirred solution of (S)-1-benzyl 2-methyl aziridine-1,2-dicarboxylate (Org. Biomol. Chem. 2005, 3, 3357) (5.02 g, 17.9 mmol) in chloroform (50 mL) and tert-butyl 2-hydroxyethyl-(methyl)carbamate (15.6 g, 89.5 mmol) at −30° C. under an atmosphere of nitrogen. The solution was stirred at room temperature overnight, then diluted with dichloromethane (20 mL) and washed with water (3×10 mL), with backwashing. The combined organic extracts were dried and evaporated in vacuo to leave the crude product which was purified by chromatography on silica using 50% diethyl ether in light petroleum as eluent to give (S)-methyl 2-(benzyloxycarbonylamino)-3-(2-(tert-butoxycarbonyl(methyl)-amino)ethoxy)propanoate as a colorless oil. Yield 3.5 g, 47%. ¹H NMR (400 MHz, CDCl₃) δ 1.36 (s, 9H), 2.77 (s, 3H), 3.23-3.47 (m, 4H), 3.60-3.68 (m, 4H), 3.81 (s, 1H), 4.17 (s, 1H), 5.05 (s, 2H), 7.24-7.30 (m, 5H).

### Step 2

### (S)-Methyl 2-(((benzyloxy)carbonyl)amino)-3-(2-(methylamino)-ethoxy)-propanoate

A mixture of (S)-methyl 2-(benzyloxycarbonylamino)-3-(2-(tert-butoxycarbonyl(methyl)amino)ethoxy)propanoate (410 mg, 1 mmol) in CH₂Cl₂ (5 mL) and TFA (5 mL) was stirred at room temperature for 1 h. Following evaporation of solvent, the product was purified by reversed phase HPLC, obtained (S)-methyl 2-(benzyloxycarbonylamino)-3-(2-(methylamino)ethoxy)propanoate. Yield 310 mg, 100%.

### Step 3

### (S)-Benzyl (4-methyl-5-oxo-1,4-oxazepan-6-yl)carbamate

To a solution of (S)-methyl 2-(benzyloxycarbonylamino)-3-(2-(methylamino)ethoxy)propanoate (310 mg, 1 mmol), ethyl acetate (88 mg, 1 mmol, 1 equiv.) in 5 mL of anhydrous dichloromethane under an atmosphere of nitrogen was added dropwise trimethyl aluminium (1 mL, 2 mmol, 2 M in hexane, 2 equiv.) at −30° C. The solution was stirred for 30 min at ambient temperature. The reaction was quenched with 5 mL 1N hydrochloric acid at −30° C., diluted with 5 mL water. The phases was separated and the aqueous extracted with dichloromethane (3×30 mL), dried over sodium sulfate, and evaporated to give a brown solid which was purified by silica gel flash chromatography eluting with 50% diethyl ether in light petroleum as eluent to give (S)-benzyl 4-methyl-5-oxo-1,4-oxazepan-6-ylcarbamate as a white solid. Yield 120 mg, 43%. ¹H NMR (400 MHz, CDCl₃) δ 3.07 (s, 3H), 3.11-3.38 (m, 1H), 3.33-3.36 (m, 1H), 3.44-3.49 (m, 1H), 3.88-4.04 (m, 3H), 4.61 (s, 1H), 4.17 (s, 1H), 5.11 (s, 2H), 6.10 (s, 1H), 7.26-7.37 (m, 5H).

### Step 4

### (S)-6-Amino-4-methyl-1,4-oxazepan-5-one

(S)-Benzyl 4-methyl-5-oxo-1,4-oxazepan-6-ylcarbamate (1000 mg, 3.59 mmol) was dissolved in MeOH (25 mL) degassed for 10 min by N₂ purge. 10% Palladium on charcoal (380 mg) was added and the mixture stirred under hydrogen for 12 hours. Filtered the reaction mixture through celite and concentrated to give a colorless oil, which was used directly in the next step. MS: m/z (obs.) 144.9 [M+H]⁺.

### Step 5

### (S)-Methyl 5-(3,3-dimethylbut-1-ynyl)-3-[N-(4-methyl-5-oxo-1,4-oxazepan-6-yl)amino]thiophene-2-carboxylate

(S)-6-Amino-4-methyl-1,4-oxazepan-5-one (0.5 g, 3.5 mmol) and cesium carbonate (3.39 g, 10.4 mmol) were dissolved in 1,4-dioxane (7.5 mL). Methyl 3-bromo-5-(3,3-dimethylbut-1-ynyl)thiophene-2-carboxylate (1.04 g, 3.48 mmol) was added and the mixture was degassed for 2 hour by N₂ purge. Pd(OAc)₂ (76 mg, 0.35 mmol) and rac-BINAP (431 mg, 0.69 mmol) were added and the mixture further purged with N₂ for 30 min. then heated to 100° C. overnight. After cooling to RT the mixture was diluted with EtOAc (10 vol), filtered through Celite and evaporated to dryness. The product was purified by silica gel chromatography (gradient of 0% to 90% EtOAc in heptane). MS: m/z (obs.) 365.5 [M+H]⁺. 1H NMR (300 MHz, CDCl₃) δ 7.77 (d, 1H), 6.70 (s, 1H), 4.28 (s, 1H), 3.95-3.79 (m, 5H), 3.78-3.42 (m, 4H), 3.10 (s, 3H), 1.32 (s, 9H).

### Step 6

### (S)-Methyl 5-(3,3-dimethylbut-1-ynyl)-3-[(trans)-4-methyl-N-(4-methyl-5-oxo-1,4-oxazepan-6-yl)cyclohexanecarboxamido]thiophene-2-carboxylate

(S)-Methyl 5-(3,3-dimethylbut-1-ynyl)-3-[N-(4-methyl-5-oxo-1,4-oxazepan-6-yl)amino]thiophene-2-carboxylate (250 mg, 0.65 mmol) was acylated with trans-4-methylcyclohexane carbonyl chloride (1.3 mmol) in the manner described for Compound 134. Yield 80 mg. MS: m/z (obs.) 489.7 [M+H]⁺. 1H NMR (300 MHz, CDCl₃) δ 7.54 (s, 1H), 7.20 (s, 1H), 5.64 (dd, 1H), 4.51 (dd, 1H), 4.36 (dd, 1H), 4.00 (d, 3H), 3.87 (d, 3H), 3.71 (dd, 1H), 3.59 (s, 1H), 3.48-3.37 (m, 1H), 3.16 (d, 2H), 3.09 (d, 3H), 1.63 (dd, 3H), 1.58 (s, 3H), 1.57-1.35 (m, 2H), 1.33 (d, 9H), 0.82 (dd, 3H), 0.70 (s, 2H).

### Step 7

### (S)-5-(3,3-Dimethylbut-1-ynyl)-3-[(trans)-4-methyl-N-(4-methyl-5-oxo-1,4-oxazepan-6-yl)cyclohexanecarboxamido]thiophene-2-carboxylic acid

(S)-Methyl 5-(3,3-dimethylbut-1-ynyl)-3-[(trans)-4-methyl-N-(4-methyl-5-oxo-1,4-oxazepan-6-yl)cyclohexanecarboxamido]thiophene-2-carboxylate (80 mg, 0.11 mmol) was hydrolyzed as described for Compound 134 to give desired 136. Yield 52 mg, 98%. MS: m/z (obs.) 475.7 [M+H]⁺. 1H NMR (300 MHz, d₆-DMSO) δ 7.41 (s, 1H), 5.40 (d, 1H), 3.99 (d, 6H), 3.27-3.02 (m, 1H), 2.92 (d, 3H), 1.56 (d, 9H), 1.29 (d, 6H), 1.23-0.97 (m, 2H), 0.77 (d, 3H), 0.69-0.43 (m, 2H).

### Preparation of Compound 137

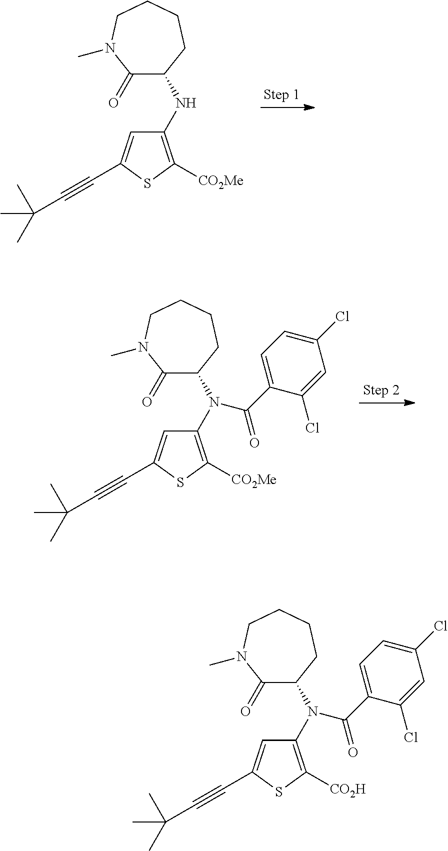 [SMILES: COC(=O)c1sc(C#CC(C)(C)C)cc1N[C@H]1CCCCN(C)C1=O ; COC(=O)c1sc(C#CC(C)(C)C)cc1N(C(=O)c1ccc(Cl)cc1Cl)[C@H]1CCCCN(C)C1=O ; CN1CCCC[C@H](N(C(=O)c2ccc(Cl)cc2Cl)c2cc(C#CC(C)(C)C)sc2C(=O)O)C1=O]

### Step 1

### (S)-Methyl 5-(3,3-dimethylbut-1-ynyl)-3-[(2,4-dichlorobenzoyl)-N-(1-methyl-2-oxoazepan-3-yl)amino]-thiophene-2-carboxylate

(S)-Methyl 5-(3,3-dimethylbut-1-ynyl)-3-[N-(1-methyl-2-oxoazepan-3-yl)amino]thiophene-2-carboxylate (160 mg, 0.42 mmol), pyridine (340 μL, 4.2 mmol), DMAP (5 mg, 0.04 mmol), and 2,4-dichlorobenzoyl chloride (176 mg, 0.84 mmol) were dissolved in DCE (5 mL) and the mixture heated to 90° C. overnight. The reaction was cooled to and diluted with 2N HCl to pH3. The organic phase was washed with brine, and dried (MgSO₄). The solution was evaporated and the residue purified by silica gel chromatography (0 to 90% EtOAc in heptane gradient). Yield: 100 mg. MS: m/z (obs.) 535.5 [M+H]⁺. 1H NMR (300 MHz, CDCl₃) δ 7.72 (s, 1H), 7.35-7.34 (m, 1H), 7.25 (t, 2H), 7.06 (dd, 1H), 5.62 (d, 1H), 3.95-3.78 (m, 4H), 3.35-3.14 (m, 1H), 3.09 (d, 3H), 1.71 (d, 4H), 1.44 (dd, 1H), 1.31 (s, 9H).

### Step 2

### (S)-5-(3,3-Dimethylbut-1-ynyl)-3-[(2,4-dichlorobenzoyl)-N-(1-methyl-2-oxoazepan-3-yl)amino]-thiophene-2-carboxylic acid

(S)-Methyl 5-(3,3-dimethylbut-1-ynyl)-3-[(2,4-dichlorobenzoyl)-N-(1-methyl-2-oxoazepan-3-yl)amino]-thiophene-2-carboxylate (100 mg, 0.19 mmol) was hydrolyzed as described for Compound 134 to give desired 137. Yield 20 mg, 19%. MS: m/z (obs.) 521.5 [M+H]⁺. 1H NMR (300 MHz, CDCl₃) δ 7.43 (d, 1H), 7.35 (d, 1H), 7.28-7.25 (m, 1H), 7.22 (s, 1H), 7.13 (d, 1H), 6.97 (s, 1H), 5.37 (d, 1H), 3.98-3.68 (m, 2H), 3.33 (t, 1H), 3.13 (d, 3H), 1.86 (s, 6H), 1.33 (m, 9H).

### Preparation of Compound 138

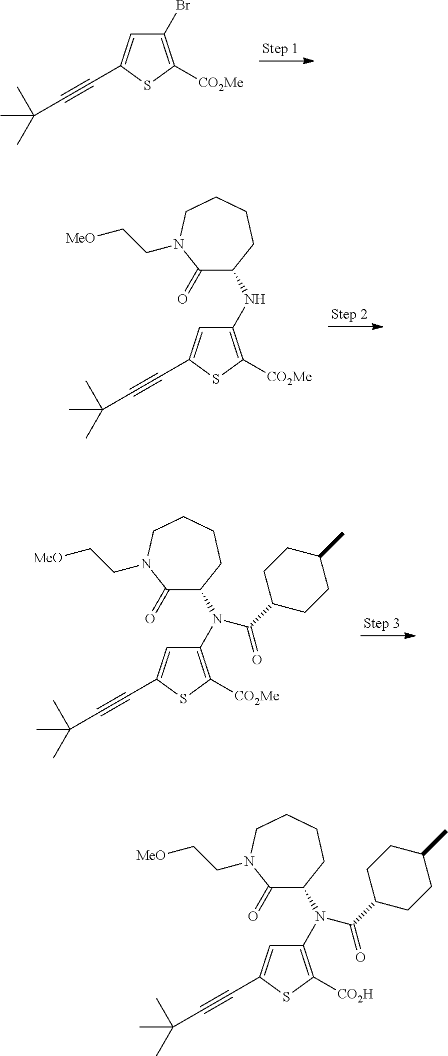

### Step 1

### (S)-Methyl 5-(3,3-dimethylbut-1-ynyl)-3-[N-(1-(2-methoxyethyl)-2-oxoazepan-3-yl)amino]thiophene-2-carboxylate

(S)-3-Amino-1-(2-methoxyethyl)-azepan-2-one hydrochloride (1.0 g, 4.5 mmol) and cesium carbonate (3.66 g, 11.2 mmol) were dissolved in 1,4-dioxane (15 mL). Methyl 3-bromo-5-(3,3-dimethylbut-1-ynyl)thiophene-2-carboxylate (1.13 g, 3.74 mmol) was added and the mixture was degassed for 2 hour by N₂ purge. Pd(OAc)₂ (84 mg, 0.37 mmol) and rac-BINAP (465 mg, 0.75 mmol) were added and the mixture further purged with N₂ for 30 min. then heated to 100° C. overnight. After cooling to RT the mixture was diluted with EtOAc (10 vol), filtered through Celite and evaporated to dryness. The product was purified by silica gel chromatography (gradient of 0% to 90% EtOAc in heptane). Yield 600 mg, 40%). MS: m/z (obs.) 407.6 [M+H]⁺. 1H NMR (300 MHz, CDCl₃) δ 6.42 (s, 1H), 4.16 (dd, 1H), 3.82 (s, 3H), 3.57 (ddd, 6H), 3.33 (s, 3H), 1.62 (s, 6H), 1.32-1.28 (m, 9H).

### Step 2

### Methyl(S)-5-(3,3-dimethylbut-1-ynyl)-3-((trans)-4-methyl-N-(1-(2-methoxyethyl)-2-oxoazepan-3-yl)cyclohexanecarboxamido)thiophene-2-carboxylate

Acylation of (S)-Methyl 5-(3,3-dimethylbut-1-ynyl)-3-[N-(1-(2-methoxyethyl)-2-oxoazepan-3-yl)amino]thiophene-2-carboxylate (300 mg) was performed as described for other examples. Yield 80 mg. MS: m/z (obs.) 531.7 [M+H]⁺. 1H NMR (300 MHz, CDCl₃) δ 7.61 (s, 1H), 5.40 (d, 1H), 3.89-3.63 (m, 5H), 3.59-3.23 (m, 8H), 2.17-1.37 (m, 14H), 1.33 (s, 9H), 0.76 (dd, 4H).

### Step 3

### (S)-5-(3,3-Dimethylbut-1-ynyl)-3-((trans)-4-methyl-N-(1-(2-methoxyethyl)-2-oxoazepan-3-yl)cyclohexanecarboxamido)thiophene-2-carboxylic acid

Methyl(S)-5-(3,3-dimethylbut-1-ynyl)-3-((trans)-4-methyl-N-(1-(2-methoxyethyl)-2-oxoazepan-3-yl)cyclohexanecarboxamido)thiophene-2-carboxylate (80 mg, 0.15 mmol) was hydrolyzed as described for Compound 134 to give desired 138. Yield 40 mg, 42%. MS: m/z (obs.) 517.7 [M+H]⁺. 1H NMR (300 MHz, CDCl₃) δ 6.74 (s, 1H), 5.09 (dd, 1H), 3.93-3.41 (m, 6H), 3.35 (d, 3H), 2.22-1.41 (m, 14H), 1.35 (d, 9H), 0.82 (dd, 3H), 0.74 (s, 1H).

### Preparation of Compound 145

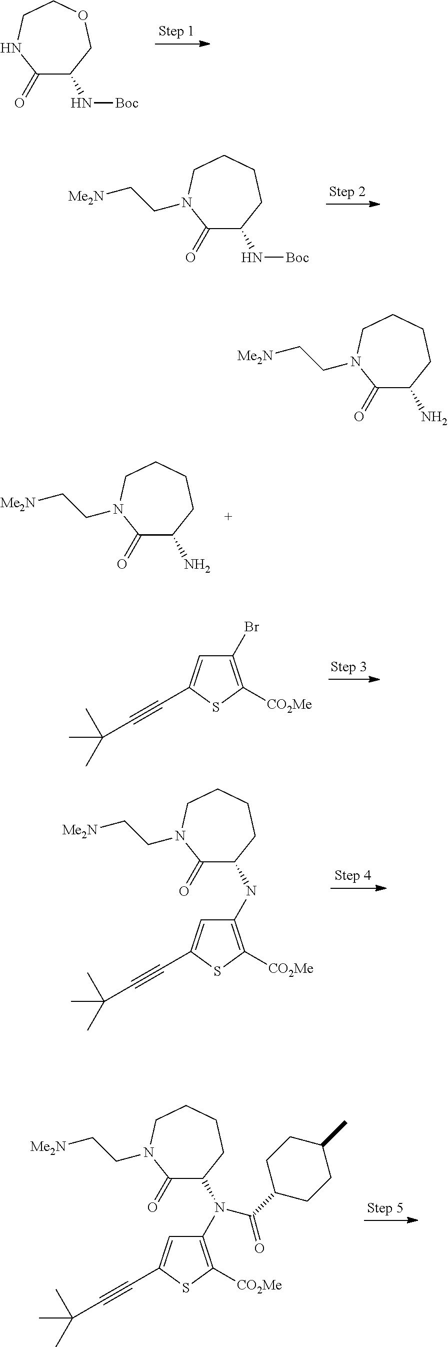

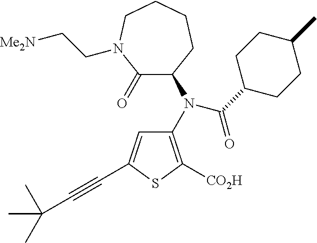 [SMILES: CN(C)CCN1CCCC[C@@H](N(c2cc(C#CC(C)(C)C)sc2C(=O)O)C(=O)[C@H]2CC[C@H](C)CC2)C1=O]

### Step 1 and 2

### (S)-3-Amino-1-(2-(dimethylamino)ethyl)azepan-2-one

Tert-butyl N-[(3S)-2-oxoazepan-3-yl]carbamate (1.0 g, 4.38 mmol) was dissolved in DMF (25 mL) and NaH (210 mg, 5.26 mmol) added at 0° C. The mixture was stirred for 1 h, then 2-bromo-N,N-dimethyl-ethanamine (1.0 g, 6.57 mmol) was added. Stirred for 24 h, then quenched by addition of aqueous NH₄Cl, diluted with EtOAc. The organic layer was washed with brine and dried with Na₂SO₄ dried and taken to the next step. The crude was taken in 4N HCl (10 mL) in dioxane and stirred for 3 hours. The clear reaction mixture became cloudy and formed white precipitate. The reaction mixture was concentrated, then redissolved in MeCN and H₂O and lyophilized. The product was used as obtained in the next step. MS: m/z (obs.) 200.2 [M+H]⁺.

### Step 3

### (S)-Methyl 5-(3,3-dimethylbut-1-ynyl)-3-[N-(1-(2-dimethylaminoethyl)-2-oxoazepan-3-yl)amino]thiophene-2-carboxylate

(S)-3-Amino-1-(2-dimethylaminoethyl)-azepan-2-one dihydrochloride (500 mg, 2.1 mmol) and cesium carbonate (1.73 g, 5.3 mmol) were dissolved in 1,4-dioxane (10 mL). Methyl 3-bromo-5-(3,3-dimethylbut-1-ynyl)thiophene-2-carboxylate (532 mg, 1.77 mmol) was added and the mixture was degassed for 2 hour by N₂ purge. Pd(OAc)₂ (40 mg, 0.18 mmol) and rac-BINAP (220 mg, 0.35 mmol) were added and the mixture further purged with N₂ for 30 min. then heated to 100° C. overnight. After cooling to RT the mixture was diluted with EtOAc and washed with water, and brine, then dried (Na₂SO₄) and evaporated to dryness. The product was purified by silica gel chromatography (gradient of 0% to 100% EtOAc in heptane, followed by 90% EtOAc, 3% Et₃N, 7% MeOH). The product eluted in the final eluent. Yield 400 mg, 35%). MS: m/z (obs.) 420.4 [M+H]⁺. 1H NMR (300 MHz, CDCl₃) δ 7.83 (d, 1H), 6.43 (s, 1H), 4.14 (d, 1H), 3.83 (s, 3H), 3.74-3.25 (m, 4H), 2.46 (d, 2H), 2.29 (d, 6H), 2.05 (d, 2H), 1.76 (d, 4H), 1.40-1.08 (m, 9H).

### Step 4

### Methyl(S)-5-(3,3-dimethylbut-1-ynyl)-3-((trans)-4-methyl-N-(1-(2-dimethylaminoethyl)-2-oxoazepan-3-yl)cyclohexanecarboxamido)thiophene-2-carboxylate

(S)-Methyl 5-(3,3-dimethylbut-1-ynyl)-3-[N-(1-(2-dimethylaminoethyl)-2-oxoazepan-3-yl)amino]thiophene-2-carboxylate (200 mg, 0.3 mmol) was dissolved in 1,2-dichloroethane (10 mL). Pyridine (40 μL, 0.49 mmol), DMAP ((4 mg, 0.03 mmol) and trans-4-methylcyclohexane carbonyl chloride (100 mg, 0.62 mmol) added. The mixture was heated to 90° C. for 48 h, then diluted with 1N HCl, EtOAC and water. The organic layer was washed with brine and dried (Na2SO4). The solvent was evaporated and the residual oil purified by silica gel chromatography, eluting with EtOAc/MeOH (90/10) followed by 3% Et3N in EtOAc/MeOH (90/10) to isolate the desired product. MS: m/z (obs.) 544.7 [M+H]⁺. 1H NMR (300 MHz, CDCl₃) δ 7.58 (s, 1H), 5.33 (d, 1H), 3.82 (s, 3H), 3.68 (dt, 1H), 3.60-3.33 (m, 2H), 3.25 (dd, 1H), 2.62 (s, 1H), 2.42 (d, 3H), 2.25 (d, 7H), 2.00 (s, 12H), 1.68 (ddd, 15H), 1.32-1.27 (m, 12H), 1.15-0.85 (m, 4H), 0.78 (d, J=6.5 Hz, 3H), 0.71-0.56 (m, 2H).

### Step 5

### (S)-5-(3,3-Dimethylbut-1-ynyl)-3-((trans)-4-methyl-N-(1-(2-dimethylaminoethyl)-2-oxoazepan-3-yl)cyclohexanecarboxamido)thiophene-2-carboxylic acid

Methyl(S)-5-(3,3-dimethylbut-1-ynyl)-3-((trans)-4-methyl-N-(1-(2-dimethylaminoethyl)-2-oxoazepan-3-yl)cyclohexanecarboxamido)thiophene-2-carboxylate (90 mg) was taken in THF (6 mL) and H₂O (2 mL) and LiOH (15 mg, 0.62 mmol) added. The mixture was stirred at ambient temperature overnight and then concentrated. The residue was diluted with EtOAc and water, and the aqueous layer separated and acidified with 1N HCl, then extracted with EtOAc. The organic layer was triturated with diethyl ether and MeCN to give 145 as a white solid. MS: m/z (obs.) 530.4 [M+H]⁺. 1H NMR (300 MHz, CD₃OD) δ 7.38 (s, 1H), 5.21 (d, 1H), 4.41-4.15 (m, 1H), 3.86-3.63 (m, 1H), 3.59-3.35 (m, 3H), 3.00 (d, 6H), 2.22-2.20 (m, 1H), 1.74 (dt, 6H), 1.33 (s, 9H), 1.17 (s, 2H), 0.82 (d, 3H), 0.77-0.48 (m, 2H).

### Preparation of Compound 146

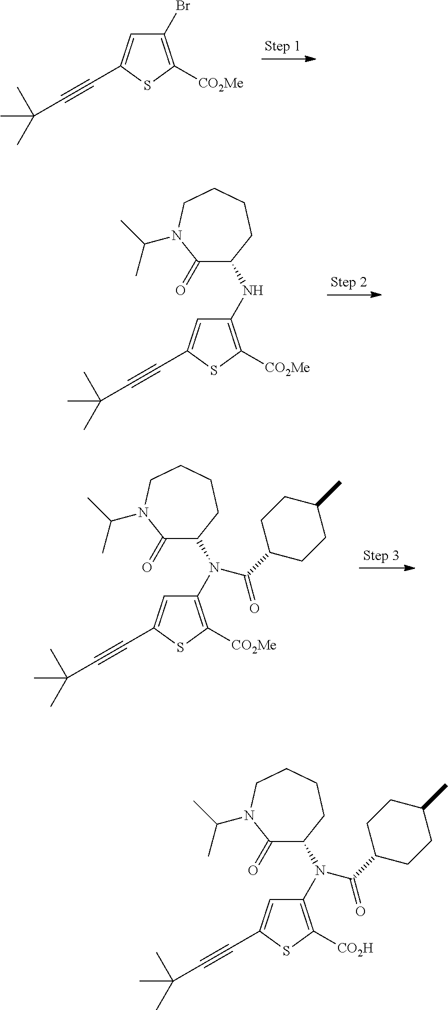

### Step 1

### (S)-Methyl 5-(3,3-dimethylbut-1-ynyl)-3-[N-(1-isopropyl-2-oxoazepan-3-yl)amino]thiophene-2-carboxylate

(S)-3-Amino-1-isopropyl-azepan-2-one hydrochloride (700 mg, 2.8 mmol) was reacted with methyl 3-bromo-5-(3,3-dimethylbut-1-ynyl)thiophene-2-carboxylate (850 mg, 3.4 mmol) as described for Compound 149. MS: m/z (obs.) 391.4 [M+H]⁺. 1H NMR (300 MHz, CDCl₃) δ 7.89 (d, 1H), 6.41 (s, 1H), 4.96 (s, 1H), 4.20-4.00 (m, 2H), 3.83 (s, 3H), 3.45-3.04 (m, 2H), 2.06-1.48 (m, 5H), 1.31 (s, 9H), 1.14 (d, 3H), 1.08 (d, 3H).

### Step 2

### Methyl(S)-5-(3,3-dimethylbut-1-ynyl)-3-((trans)-4-methyl-N-(1-isopropyl-2-oxoazepan-3-yl)cyclohexanecarboxamido)thiophene-2-carboxylate

Acylation of (S)-methyl 5-(3,3-dimethylbut-1-ynyl)-3-[N-(1-isopropyl-2-oxoazepan-3-yl)amino]thiophene-2-carboxylate (170 mg, 0.45 mmol) with trans-4-methylcyclohexane carbonyl chloride (140 mg, 0.87 mmol) was performed as described for Compound 149. The product was purified by silica gel chromatography (ISCO column, eluted with a gradient of 0% to 90% EtOAc in heptane). MS: m/z (obs.) 515.4 [M+H]⁺. 1H NMR (300 MHz, CDCl₃) δ 7.61 (s, 1H), 5.34 (d, 1H), 4.88 (s, 1H), 3.82 (s, 3H), 3.45-3.20 (m, 2H), 2.30-2.12 (m, 1H), 2.01-1.32 (m, 17H), 1.30 (d, 9H), 1.09 (d, 6H), 0.94 (d, 2H), 0.88 (t, 3H), 0.79 (s, 1H), 0.72-0.55 (m, 1H).

### Step 3

### (S)-5-(3,3-Dimethylbut-1-ynyl)-3-((trans)-4-methyl-N-(1-isopropyl-2-oxoazepan-3-yl)cyclohexanecarboxamido)thiophene-2-carboxylic acid

Methyl(S)-5-(3,3-dimethylbut-1-ynyl)-3-((trans)-4-methyl-N-(1-isopropyl-2-oxoazepan-3-yl)cyclohexanecarboxamido)thiophene-2-carboxylate was hydrolyzed as described for Compound 149 to give desired 146. MS: m/z (obs.) 501.4 [M+H]⁺. 1H NMR (300 MHz, CDCl₃) 6.65 (s, 1H), 5.00-4.92 (m, 1H), 4.84 (s, 2H), 3.51-3.27 (m, 2H), 2.28-2.11 (m, 1H), 2.07-1.87 (m, 3H), 1.86-1.69 (m, 4H), 1.67-1.44 (m, 4H), 1.49-1.31 (m, 3H), 1.26 (d, 9H), 1.06 (dt, 6H), 0.81 (s, 1H), 0.75-0.69 (m, 3H), 0.66 (s, 2H).

### Preparation of Compound 147

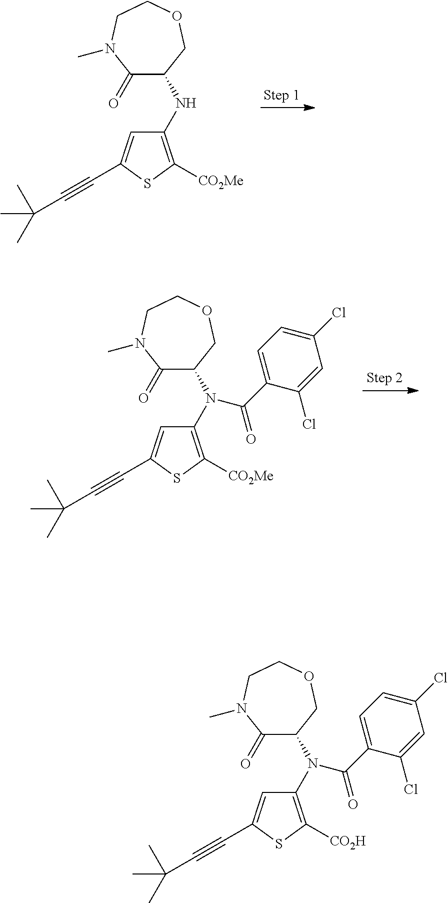 [SMILES: COC(=O)c1sc(C#CC(C)(C)C)cc1N[C@H]1COCCN(C)C1=O ; COC(=O)c1sc(C#CC(C)(C)C)cc1N(C(=O)c1ccc(Cl)cc1Cl)[C@H]1COCCN(C)C1=O ; CN1CCOC[C@H](N(C(=O)c2ccc(Cl)cc2Cl)c2cc(C#CC(C)(C)C)sc2C(=O)O)C1=O]

### Step 1

### (S)-Methyl 5-(3,3-dimethylbut-1-ynyl)-3-[(2,4-dichlorobenzoyl)-N-(4-methyl-5-oxo-1,4-oxazepan-6-yl)amino]thiophene-2-carboxylate

(S)-Methyl 5-(3,3-dimethylbut-1-ynyl)-3-[N-(1-ethyl-2-oxoazepan-3-yl)amino]thiophene-2-carboxylate (100 mg, 0.26 mmol), pyridine (34 μL, 0.42 mmol), DMAP (3 mg, 0.03 mmol), and 2,4-dichlorobenzoyl chloride (0.5 mmol) were dissolved in DCE (10 mL) and the mixture heated to 90° C. overnight. The reaction was worked up as described for Compound 137. Yield 100 mg. MS: m/z (obs.) 537.1 [M+H]⁺.

### Step 2

### (S)-5-(3,3-Dimethylbut-1-ynyl)-3-[(2,4-dichlorobenzoyl)-N-(4-methyl-5-oxo-1,4-oxazepan-6-yl)amino]thiophene-2-carboxylic acid

(S)-Methyl 5-(3,3-dimethylbut-1-ynyl)-3-[(2,4-dichlorobenzoyl)-N-(4-methyl-5-oxo-1,4-oxazepan-6-yl)amino]thiophene-2-carboxylate (0.19 mmol) was hydrolyzed as described for Compound 137 to give desired 147. Yield 97 mg. MS: m/z (obs.) 523.2 [M+H]⁺. 1H NMR (300 MHz, CDCl₃) δ 7.64-7.54 (m, 1H), 7.58 (d, 1H), 7.33 (s, 1H), 7.27-7.20 (m, 1H), 7.17 (dd, 5H), 7.04 (s, 0.5H), 4.63 (d, 1H), 4.38 (s, 2H), 4.06 (s, 1H), 3.90 (dd, 1H), 3.69 (s, 1H), 3.31 (m, 1H), 3.18-3.08 (m, 3H), 1.34-1.24 (m, 9H).

### Preparation of Compound 117

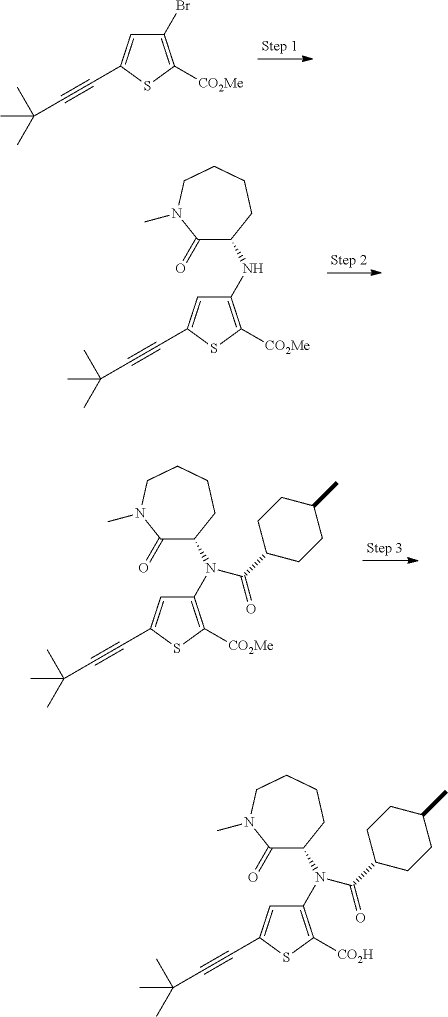

### Step 1

### (S)-Methyl 5-(3,3-dimethylbut-1-yn-1-yl)-3-((1-methyl-2-oxopiperidin-3-yl)amino)thiophene-2-carboxylate

To a degassed suspension of methyl 3-bromo-5-(3,3-dimethylbut-1-yn-1-yl)thiophene-2-carboxylate (1.18 g, 3.90 mmol), cesium carbonate (1.27 g, 3.90 mmol), and (S)-3-amino-1-methylpiperidin-2-one (0.60 g, 4.7 mmol) in toluene (12 mL) was added palladium acetate (88 mg, 0.39 mmol) followed by (±)-BINAP (243 mg, 0.39 mmol). The reaction mixture was heated to 90° C. and stirred until the starting material was consumed (monitored by HPLC and LCMS). The reaction mixture was cooled to room temperature, filtered through Celite, and the Celite pad rinsed with dichloromethane. The solvent was removed under reduced pressure and the crude product purified by column chromatography, eluting with 0-90% ethyl acetate/hexanes to afford the product as a 90:10 mixture of the S and R isomers (1.03 g). Crystallization from toluene provided desired (S)-methyl 5-(3,3-dimethylbut-1-yn-1-yl)-3-((1-methyl-2-oxopiperidin-3-yl)amino)thiophene-2-carboxylate (453 mg, 45%). MS: m/z (obs.) 349.13 [M+H]⁺. ¹H NMR (300 MHz, MeOD) δ 6.80 (s, 1H), 4.10 (dd, J=10.3, 5.6 Hz, 1H), 3.76 (s, 3H), 3.47-3.35 (m, 2H), 2.94 (s, 3H), 2.28 (dt, J=9.5, 5.1 Hz, 1H), 2.05-1.90 (m, 2H), 1.86-1.68 (m, 1H), 1.30 (s, 9H). Chiral HPLC: >99:1 S/R ratio (Chiralpak AD-H, 40% EtOH/Hex).

### Step 2

### Methyl 5-(3,3-dimethylbut-1-yn-1-yl)-3-(trans-4-methyl-N—((S)-1-methyl-2-oxopiperidin-3-yl)cyclohexanecarboxamido)thiophene-2-carboxylate

(S)-methyl 5-(3,3-dimethylbut-1-yn-1-yl)-3-((1-methyl-2-oxopiperidin-3-yl)amino)thiophene-2-carboxylate (670 mg, 1.92 mmol) was dissolved in DCE (7 mL). Pyridine (156 μL, 1.92 mmol), DMAP (23 mg, 0.19 mmol), and trans-4-methylcyclohexanecarbonyl chloride (772 g, 4.80 mmol) were added and the reaction mixture heated to 90° C. until the starting material was consumed (monitored by TLC and HPLC). The reaction mixture was cooled to RT and washed with 2N HCl, sat. NaHCO₃, and brine. The organic layer was dried (Na₂SO₄), filtered, and the solvent removed under reduced pressure. The crude product was purified by column chromatography, eluting with 0-90% ethyl acetate/hexanes, to afford methyl 5-(3,3-dimethylbut-1-yn-1-yl)-3-(trans-4-methyl-N—((S)-1-methyl-2-oxopiperidin-3-yl)cyclohexanecarboxamido)thiophene-2-carboxylate (850 mg, 93%). MS: m/z (obs.) 473.47 [M+H]⁺. 1H NMR (300 MHz, MeOD) δ 7.16 (d, J=82.0 Hz, 1H), 5.41 (dd, J=12.3, 5.6 Hz, 1H), 3.82 (d, J=1.6 Hz, 3H), 3.79-3.72 (m, 1H), 3.50 (td, J=12.0, 3.6 Hz, 1H), 3.26 (dd, J=8.6, 4.3 Hz, 1H), 2.94 (d, J=6.2 Hz, 3H), 2.46 (ddd, J=16.6, 14.1, 3.7 Hz, 1H), 2.18-1.36 (m, 8H), 1.33 (s, 9H), 0.81 (d, J=6.5 Hz, 3H), 0.68 (dd, J=20.7, 11.0 Hz, 2H).

### Step 3

### 5-(3,3-dimethylbut-1-yn-1-yl)-3-(trans-4-methyl-N—((S)-1-methyl-2-oxopiperidin-3-yl)cyclohexanecarboxamido)thiophene-2-carboxylic acid

Methyl 5-(3,3-dimethylbut-1-yn-1-yl)-3-(trans-4-methyl-N—((S)-1-methyl-2-oxopiperidin-3-yl)cyclohexanecarboxamido)thiophene-2-carboxylate (850 mg, 1.80 mmol) was dissolved in THF (4.3 mL) and water (4.3 mL) and lithium hydroxide (129 mg, 5.39 mmol) added. The reaction mixture was stirred at room temperature until the starting material was consumed (monitored by TLC). The reaction mixture was acidified with 3N HCl and washed with ethyl acetate and brine. The organic layer was dried (Na₂SO₄), filtered, and the solvent removed under reduced pressure to give the crude product as a pale yellow foam which was crystallized from MTBE/heptane to afford compound 117 (695 mg, 84%). MS: m/z (obs.) 459.24 [M+H]⁺, 457.19 [M−H]⁻. ¹H NMR (300 MHz, MeOD) δ 7.15 (d, J=78.0 Hz, 1H), 5.28 (dd, J=12.1, 5.6 Hz, 1H), 3.76 (dd, J=11.1, 6.7 Hz, 1H), 3.49 (td, J=12.1, 3.9 Hz, 1H), 2.95 (d, J=2.7 Hz, 3H), 2.46 (ddd, J=16.7, 14.1, 3.6 Hz, 1H), 2.23-2.01 (m, 2H), 1.99-1.84 (m, 1H), 1.83-1.38 (m, 6H), 1.33 (s, 9H), 0.81 (d, J=6.5 Hz, 3H), 0.77-0.58 (m, 2H). Chiral HPLC: 99:1 S/R (Chiralpak IC, 100% ACN/0.1% TFA).

### Preparation of Compound 122

### Step 1

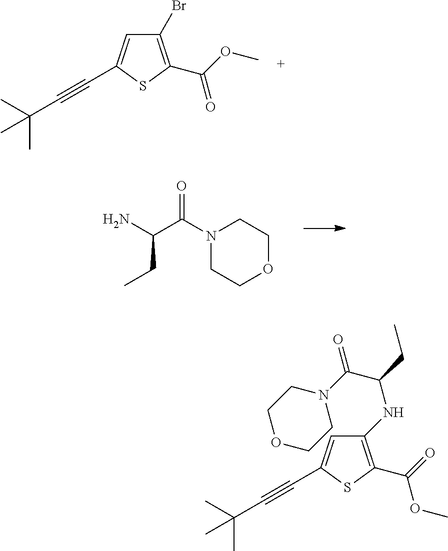 [SMILES: COC(=O)c1sc(C#CC(C)(C)C)cc1Br ; CC[C@@H](N)C(=O)N1CCOCC1 ; CC[C@@H](Nc1cc(C#CC(C)(C)C)sc1C(=O)OC)C(=O)N1CCOCC1]

### Methyl 5-(3,3-dimethylbut-1-ynyl)-3-[[(1R)-1-(morpholine-4-carbonyl)propyl]amino]thiophene-2-carboxylate

A suspension of methyl-3-bromo-5-(3,3-dimethylbut-1-ynyl)thiophene-2-carboxylate (2 g, 6.64 mmol), (R)-2-amino-1-morpholinobutan-1-one (2.06 g, 9.96 mmol), Cs₂CO₃ (6.5 g, 20 mmol) in toluene (40 mL) was deoxygenated by purging a stream of argon for 60 min, and Pd(OAc)₂ (150 mg, 0.664 mmol) and (±)BINAP (413 mg, 0.664 mmol) were added. The purging was continued for another 30 min and then the mixture was stirred at 100° C. for 16 h. Reaction progress was monitored by TLC. Upon completion the reaction mixture was cooled to RT, diluted with EtOAc (100 mL) and filtered through celite. The filtrate was washed with water (2×35 mL), brine (30 mL), dried over Na₂SO₄, and concentrated. The resulting crude product was purified by column chromatography (100-200 mesh silica gel, 20% EtOAc in petroleum ether) to afford methyl 5-(3,3-dimethylbut-1-ynyl)-3-[[(1R)-1-(morpholine-4-carbonyl)propyl]amino]thiophene-2-carboxylate (1.6 g, 61%, yellow solid). TLC: 40% EtOAc in petroleum ether, R_{f}: 0.41. Analysis by LCMS: MH⁺=393.2; ¹H NMR (400 MHz, CDCl₃): 7.33 (d, J=8 Hz; exchanged with D₂O; 1H), 6.53 (s, 1H), 4.21-4.20 (m, 1H), 3.81 (s, 3H), 3.67-3.54 (m, 8H), 1.89-1.86 (m, 1H), 1.76-1.71 (m, 1H), 1.30 (s, 9H), 0.99 (t, J=7.6 Hz; 3H).

### Step 2

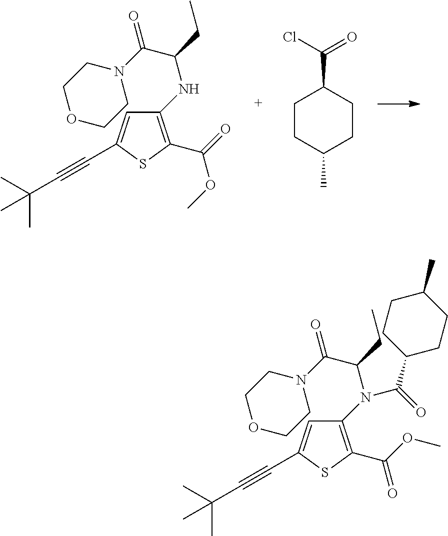

### Methyl 5-(3,3-dimethylbut-1-ynyl)-3-[(trans-4-methylcyclohexanecarbonyl)-[(1R)-1-(morpholine-4-carbonyl)propyl]amino]thiophene-2-carboxylate

A solution of methyl 5-(3,3-dimethylbut-1-ynyl)-3-[[(1R)-1-(morpholine-4-carbonyl)propyl]amino]thiophene-2-carboxylate (1 g, 2.6 mmol) in dichloroethane (50 mL) at 0° C. was stirred with pyridine (15 mL, 15 vol) and DMAP (156 mg, 1.275 mmol). To this solution trans 4-methyl cylohexane carbonyl chloride (6.14 g, 38.3 mmol) in dichloroethane (20 mL) was added dropwise. After the addition was complete, the reaction mixture was stirred at 100° C. for 16 h. Reaction progress was monitored by TLC. Upon completion the reaction mixture was diluted with dichloromethane (100 mL), washed with 2N aq. HCl (2×30 mL), water (30 mL), 10% NaHCO₃ solution (2×30 mL), brine (30 mL), dried over Na₂SO₄, concentrated and the residue was purified by column chromatography (100-200 mesh silica gel, 10% EtOAc in petroleum ether) to afford methyl 5-(3,3-dimethylbut-1-ynyl)-3-[(trans-4-methylcyclohexanecarbonyl)-[(1R)-1-(morpholine-4-carbonyl)propyl]amino]thiophene-2-carboxylate (1 g, 76%, yellow solid). TLC: 40% EtOAc in petroleum ether, R_{f}: 0.45. Analysis by LCMS (column: BEH C-18 (2.1×50 mm), mobile phase: A: 0.01 M aq. NH₄HCO₃, B: MeCN, time (min)/% B gradient: 0 min/40%, 2/95, 4/95, 4.1/20, flow: 0.4 mL/min, sample diluent: MeCN): MH⁺=517.2, t_{R}=2.68 min. ¹H NMR (400 MHz, CDCl₃): 7.42 (s, 1H), 5.25-5.19 (m, 1H), 3.83-3.58 (m, 12H), 2.0 (bs, 1H), 1.66-1.59 (m, 6H), 1.42-1.31 (m, 9H), 0.91-0.78 (m, 8H), 0.69-0.66 (m, 3H).

### Step 3

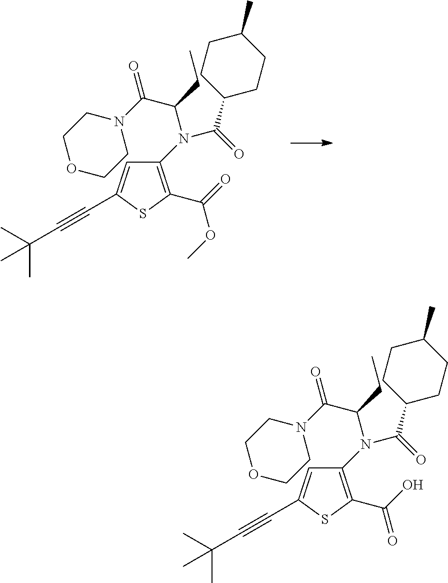 [SMILES: CC[C@H](C(=O)N1CCOCC1)N(c1cc(C#CC(C)(C)C)sc1C(=O)OC)C(=O)[C@H]1CC[C@H](C)CC1 ; CC[C@H](C(=O)N1CCOCC1)N(c1cc(C#CC(C)(C)C)sc1C(=O)O)C(=O)[C@H]1CC[C@H](C)CC1]

### 5-(3,3-dimethylbut-1-ynyl)-3-[(trans-4-methylcyclohexanecarbonyl)-[(1R)-1-(morpholine-4-carbonyl)propyl]amino]thiophene-2-carboxylic acid

To a stirred solution of methyl 5-(3,3-dimethylbut-1-ynyl)-3-[(trans-4-methylcyclohexanecarbonyl)-[(1R)-1-(morpholine-4-carbonyl)propyl]amino]thiophene-2-carboxylate (70 mg, 0.14 mmol) in a mixture of THF and water (1:1) (4 mL), LiOH.H₂O (28.3 mg, 0.675 mmol) was added at RT and stirred for 3 h. Reaction progress was monitored by TLC. Upon completion the reaction mixture was acidified (pH˜1) with 2N aq. HCl and extracted with EtOAc (30 mL). The combined EtOAc layer was washed with water (3×10 mL), brine (10 mL), dried over Na₂SO₄ and concentrated. The residue was purified by preparative TLC (5% MeOH—CHCl₃) to afford 122, 5-(3,3-dimethylbut-1-ynyl)-3-[(trans-4-methylcyclohexanecarbonyl)-[(1R)-1-(morpholine-4-carbonyl)propyl]amino]thiophene-2-carboxylic acid (25 mg, 37%, white solid). TLC: 10% MeOH in CHCl₃, R_{f}: 0.38. Analysis by LCMS (column: Zorbax SB-CN (250×4.6 mm, 5 μm), mobile phase: A: 0.01 M aq. HCOONH₄, B: MeCN, time (min)/% B gradient: 0 min/30%, 8/90, 15/90,15.1/30, flow: 1.04 mL/min, sample diluent: MeCN): MH⁺=503.2, t_{R}=5.36 min. ¹H NMR (400 MHz, DMSO-d6): 13.65 (bs, exchanged with D₂O; 1H), 7.06 (bs, 1H), 5.15 (bs, 1H), 3.69-3.44 (m, 8H), 2.08-2.04 (m, 1H), 1.68-1.38 (m, 6H), 1.35-0.87 (m, 12H), 0.85-0.53 (m, 8H).

### Preparation of Compound 123

### Step 1

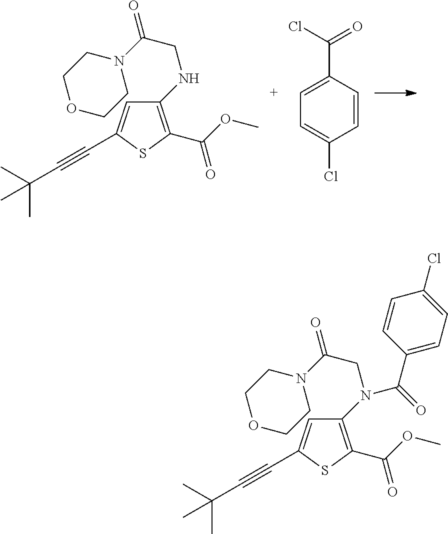

### Methyl 3-[(4-chlorobenzoyl)-(2-morpholino-2-oxo-ethyl)amino]-5-(3,3-dimethylbut-1-ynyl)thiophene-2-carboxylate

To a stirred solution of methyl 5-(3,3-dimethylbut-1-ynyl)-3-(2-morpholino-2-oxoethylamino)thiophene-2-carboxylate (200 mg, 0.55 mmol) in dichloroethane (10 mL) at 0° C. was added pyridine (2 mL, 10 vol) and DMAP (33.6 mg, 0.275 mmol). To this solution, 4-Chlorobenzoyl chloride (0.7 mL, 5.5 mmol) was added dropwise. After addition the reaction mixture was stirred at 100° C. for 16 h. Reaction progress was monitored by TLC. Upon completion, the reaction mixture was diluted with EtOAc (80 mL), washed with 2N aq. HCl (2×30 mL), water (30 mL), 10% NaHCO₃ solution (2×30 mL), brine (30 mL), dried over Na₂SO₄ and concentrated. The residue was purified by column chromatography (100-200 mesh silica gel, 50% EtOAc in petroleum ether) to afford methyl 3-[(4-chlorobenzoyl)-(2-morpholino-2-oxo-ethyl)amino]-5-(3,3-dimethylbut-1-ynyl)thiophene-2-carboxylate (190 mg, 67%, white solid). TLC: 50% EtOAc in petroleum ether, R_{f}: 0.31. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=503.2, t_{R}=4.65 min. ¹H NMR (400 MHz, DMSO-d6): 7.38-7.26 (m, 4H), 7.11 (s, 1H), 5.11 (d, J=16.8 Hz; 1H), 4.23 (d, J=16.8 Hz; 1H) 3.70 (s, 3H), 3.60-3.46 (m, 8H), 1.27 (s, 9H).

### Step 2

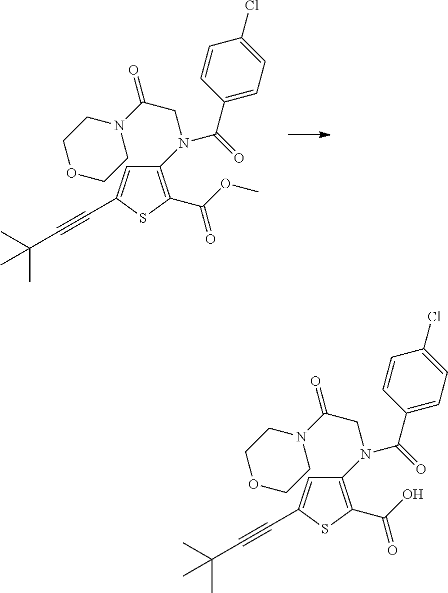 [SMILES: COC(=O)c1sc(C#CC(C)(C)C)cc1N(CC(=O)N1CCOCC1)C(=O)c1ccc(Cl)cc1 ; CC(C)(C)C#Cc1cc(N(CC(=O)N2CCOCC2)C(=O)c2ccc(Cl)cc2)c(C(=O)O)s1]

### 3-[(4-chlorobenzoyl)-(2-morpholino-2-oxo-ethyl)amino]-5-(3,3-dimethylbut-1-ynyl)thiophene-2-carboxylic acid

To a stirred solution of methyl 3-[(4-chlorobenzoyl)-(2-morpholino-2-oxo-ethyl)amino]-5-(3,3-dimethylbut-1-ynyl)thiophene-2-carboxylate (190 mg, 0.377 mmol) in a mixture (1:1) of THF and water (4 mL), LiOH.H₂O (47.5 mg, 1.13 mmol) was added at RT and the mixture stirred for 16 h. Reaction progress was monitored by TLC. Upon completion, the reaction mixture was acidified (pH˜1) with 1M aq. HCl and extracted with EtOAc (60 mL). The organic layer was washed with water (3×20 mL), brine (20 mL), dried over Na₂SO₄ and concentrated. The residue was purified by trituration with Et₂O (2×50 mL) to afford 123, 3-[(4-chlorobenzoyl)-(2-morpholino-2-oxo-ethyl)amino]-5-(3,3-dimethylbut-1-ynyl)thiophene-2-carboxylic acid (139.3 mg, 75%, green solid). TLC: 10% MeOH in CHCl₃, R_{f}: 0.38. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=489.0, t_{R}=4.21 min. ¹H NMR (400 MHz, DMSO-d6): 13.47 (br s, exchanged with D₂O; 1H), 7.37 (d, J=8.4 Hz; 2H), 7.26 (d, J=8.4 Hz; 2H), 7.12 (s, 1H), 5.12-5.08 (m, 1H), 4.24-4.20 (m, 1H), 3.59-3.46 (m, 8H), 1.27 (s, 9H).

### Preparation of Compound 124

### Step 1

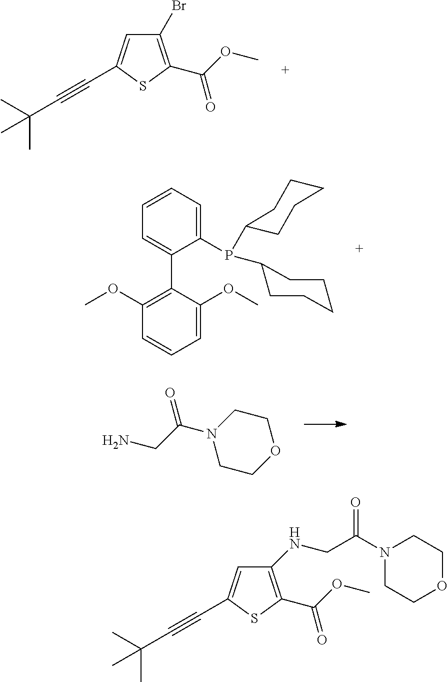

### Methyl 5-(3,3-dimethylbut-1-ynyl)-3-[(2-morpholino-2-oxo-ethyl)amino]thiophene-2-carboxylate

A 100 mL 2-neck round bottomed flask was charged with 2-amino-1-morpholino-ethanone hydrochloride (620 mg, 3.43 mmol), methyl 3-bromo-5-(3,3-dimethylbut-1-ynyl)thiophene-2-carboxylate (1.03 g, 3.43 mmol) and Cs₂CO₃ (3.35 g, 10.3 mmol). Dioxane (30 mL) was added and nitrogen bubbled through the suspension for 30 min. Pd₂(dba)₃ was then added (56.5 mg, 0.0617 mmol) followed by dicyclohexyl-[2-(2,6-dimethoxyphenyl)phenyl]phosphane (“sPHOS”, 102 mg, 0.248 mmol). The reaction was heated to 105° C. overnight; based on partial conversion by LCMS, 200 mg sPHOS, 112 mg Pd₂(dba)₃, and 10 mL dioxane were added and the reaction run an additional 3 days at 100° C., after which LCMS analysis showed complete conversion. The mixture was cooled, filtered through Celite, and concentrated. Chromatography on silica followed by concentration in vacuo gave the desired product as a white solid which precipitated during concentration. Analysis by LCMS (column: C4, mobile phase: A: 0.1% aq. HCOOH, B: MeCN, time (min)/% B gradient: 0 min/60%, 7/98, flow rate: 1.0 mL/min): MH⁺=365.2.0, t_{R}=1.32 min. ¹H NMR (300 MHz, CDCl₃): 7.38 (br s, 1H), 6.58 (s, H), 4.03 (d, 2H), 3.85 (s, 3H), 3.70 (br s, 6H), 3.45 (br s, 2H), 1.30 (s, 9H).

### Step 2

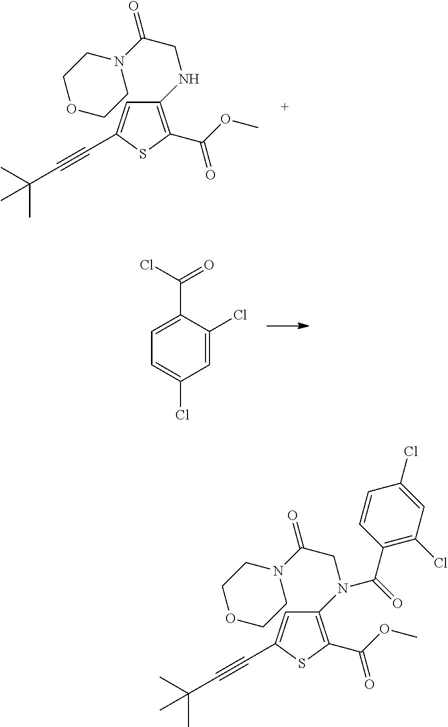 [SMILES: COC(=O)c1sc(C#CC(C)(C)C)cc1NCC(=O)N1CCOCC1 ; O=C(Cl)c1ccc(Cl)cc1Cl ; COC(=O)c1sc(C#CC(C)(C)C)cc1N(CC(=O)N1CCOCC1)C(=O)c1ccc(Cl)cc1Cl]

### Methyl 3-[(2,4-dichlorobenzoyl)-(2-morpholino-2-oxo-ethyl)amino]-5-(3,3-dimethylbut-1-ynyl)thiophene-2-carboxylate

To a stirred solution of methyl 5-(3,3-dimethylbut-1-ynyl)-3-(2-morpholino-2-oxoethylamino)thiophene-2-carboxylate (200 mg, 0.55 mmol) in dichloroethane (10 mL) at 0° C. was stirred with pyridine (2 mL, 10 vol) and DMAP (33.6 mg, 0.275 mmol). To this solution, 2,4-dichlorobenzoyl chloride (0.77 mL, 5.5 mmol) was added dropwise. After addition the reaction mixture was stirred at 100° C. for 16 h. Reaction progress was monitored by TLC. Upon completion, the reaction mixture was diluted with EtOAc (80 mL), washed with 2N aq. HCl (2×30 mL), water (30 mL), 10% NaHCO₃ solution (2×30 mL), brine (30 mL), dried over Na₂SO₄ and concentrated. The residue was purified by column chromatography (100-200 mesh silica gel, 50% EtOAc in petroleum ether) to afford methyl 3-[(2,4-dichlorobenzoyl)-(2-morpholino-2-oxo-ethyl)amino]-5-(3,3-dimethylbut-1-ynyl)thiophene-2-carboxylate (180 mg, 60%, white solid). TLC: 50% EtOAc in petroleum ether, R_{f}: 0.38. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10%, flow rate: 1.0 mL/min, sample diluent: MeCN): MH⁺=537.2, t_{R}=4.78 min. ¹H NMR (400 MHz, DMSO-d6): 7.62 (s, 1H), 7.37-7.35 (m, 1H), 7.22-7.20 (m, 1H), 5.15 (d, J=16.4 Hz; 1H), 4.21 (d, J=16.8 Hz; 1H), 3.83 (s, 3H), 3.58-3.44 (m, 8H), 1.24 (s, 9H).

### Step 3

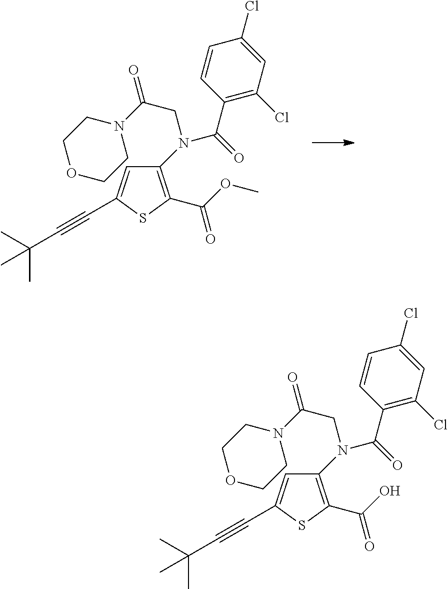 [SMILES: COC(=O)c1sc(C#CC(C)(C)C)cc1N(CC(=O)N1CCOCC1)C(=O)c1ccc(Cl)cc1Cl ; CC(C)(C)C#Cc1cc(N(CC(=O)N2CCOCC2)C(=O)c2ccc(Cl)cc2Cl)c(C(=O)O)s1]

### 3-[(2,4-dichlorobenzoyl)-(2-morpholino-2-oxo-ethyl)amino]-5-(3,3-dimethylbut-1-ynyl)thiophene-2-carboxylic acid

To a stirred solution of methyl 3-[(2,4-dichlorobenzoyl)-(2-morpholino-2-oxo-ethyl)amino]-5-(3,3-dimethylbut-1-ynyl)thiophene-2-carboxylate (180 mg, 0.335 mmol) in a mixture (1:1) of THF and water (4 mL), LiOH.H₂O (52.3 mg, 1.24 mmol) was added at RT, and the mixture stirred for 16 h. Reaction progress was monitored by TLC. Upon completion the reaction mixture was acidified (pH˜1) with 1M aq. HCl, extracted with EtOAc (60 mL). The organic layer was washed with water (3×20 mL), brine (20 mL), dried over Na₂SO₄ and concentrated. The crude residue purified by washing with Et₂O (2×5 mL) to afford 124, 3-[(2,4-dichlorobenzoyl)-(2-morpholino-2-oxo-ethyl)amino]-5-(3,3-dimethylbut-1-ynyl)thiophene-2-carboxylic acid (139 mg, 79%, white solid). TLC: 10% MeOH in CHCl₃, R_{f}: 0.39. Analysis by LCMS (column: Zodiac Sil 120-3-C-18-aq. (4.6×50 mm), mobile phase: A: 0.01M aq. HCOONH₄, B: MeOH, time (min)/% B gradient: 0 min/60%, 3/90, 8/90, 8.01/60, flow rate: 1.0 mL/min, sample diluent: MeOH/MeCN): MH⁺=523.0, t_{R}=2.73 min. ¹H NMR (400 MHz, DMSO-d6): 13.7 (br s, exchanged with D₂O; 1H), 7.62-7.61 (m, 1H), 7.40-7.38 (m, 1H), 7.23-7.20 (m, 1H), 7.12 (s, 1H), 5.15-5.11 (m, 1H), 4.23-4.19 (m, 1H), 3.58-3.44 (m, 8H), 1.24 (s, 9H).

### Preparation of Compound 125

### Step 1

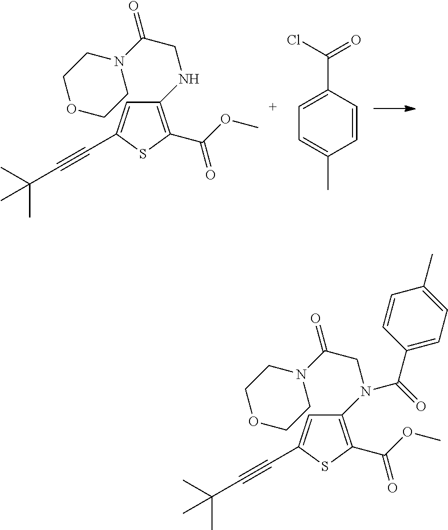

### Methyl 5-(3,3-dimethylbut-1-ynyl)-3-[(4-methylbenzoyl)-(2-morpholino-2-oxo-ethyl)amino]thiophene-2-carboxylate

A stirred solution of methyl 5-(3,3-dimethylbut-1-ynyl)-3-(2-morpholino-2-oxoethylamino)thiophene-2-carboxylate (200 mg, 0.55 mmol) in dichloroethane (10 mL) at 0° C. was stirred with pyridine (2 mL, 10 vol) and DMAP (33.6 mg, 0.275 mmol). To this solution 4-methylbenzoyl chloride (0.73 mL, 5.5 mmol) was added dropwise and the reaction mixture stirred at 100° C. for 16 h. Reaction progress was monitored by TLC. On completion, the reaction mixture was diluted with EtOAc (80 mL), washed with 2N aq. HCl (2×30 mL), water (30 mL), 10% NaHCO₃ solution (2×30 mL), brine (30 mL), dried over Na₂SO₄ and concentrated. The residue was purified by column chromatography (100-200 mesh silica gel, 50% EtOAc in petroleum ether) to afford methyl 5-(3,3-dimethylbut-1-ynyl)-3-[(4-methylbenzoyl)-(2-morpholino-2-oxo-ethyl)amino]thiophene-2-carboxylate (200 mg, 75%, white solid). TLC: 50% EtOAc in petroleum ether, R_{f}: 0.29. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=483.2, t_{R}=4.49 min. ¹H NMR (400 MHz, DMSO-d6): 7.16-7.06 (m, 5H), 5.10 (d, J=16.8 Hz; 1H), 4.18 (d, J=16.4 Hz; 1H), 3.71 (s, 3H), 3.58-3.57 (m, 4H), 3.45 (br s, 4H), 1.26 (s, 9H).

### Step 2

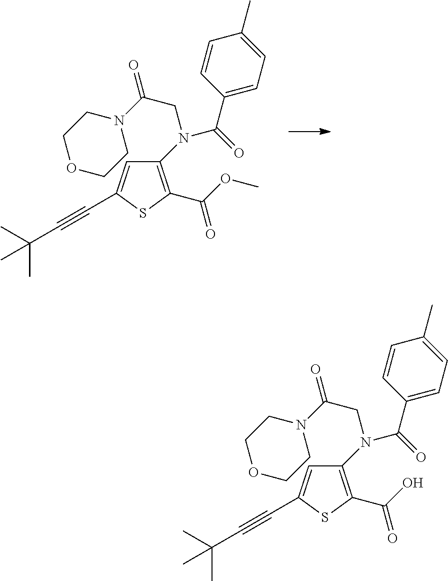 [SMILES: COC(=O)c1sc(C#CC(C)(C)C)cc1N(CC(=O)N1CCOCC1)C(=O)c1ccc(C)cc1 ; Cc1ccc(C(=O)N(CC(=O)N2CCOCC2)c2cc(C#CC(C)(C)C)sc2C(=O)O)cc1]

### 5-(3,3-dimethylbut-1-ynyl)-3-[(4-methylbenzoyl)-(2-morpholino-2-oxo-ethyl)amino]thiophene-2-carboxylic acid

To a stirred solution of methyl 5-(3,3-dimethylbut-1-ynyl)-3-[(4-methylbenzoyl)-(2-morpholino-2-oxo-ethyl)amino]thiophene-2-carboxylate (200 mg, 0.415 mmol) in a mixture (1:1) of THF and water (4 mL), LiOH.H₂O (52.3 mg, 1.24 mmol) was added at RT and the mixture stirred for 16 h. Reaction progress was monitored by TLC. On completion, the reaction mixture was acidified (pH˜1) with 1M aq. HCl, extracted with EtOAc (60 mL). The organic layer was washed with water (3×20 mL), brine (20 mL), dried over Na₂SO₄ and concentrated. The residue was purified by trituration with Et₂O (2×5 mL) to afford 125, 5-(3,3-dimethylbut-1-ynyl)-3-[(4-methylbenzoyl)-(2-morpholino-2-oxo-ethyl)amino]thiophene-2-carboxylic acid (140 mg, 72%, white solid). TLC: 10% MeOH in CHCl₃, R_{f}: 0.44. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH-=467.0, t_{R}=4.08 min. ¹H NMR (400 MHz, DMSO-d6): 13.4 (br s, exchanged with D₂O; 1H), 7.16-7.06 (m, 5H), 5.11-5.06 m, 1H), 4.21-4.17 (m, 1H), 3.59-3.46 (m, 8H), 2.25 (s, 3H), 1.26 (s, 9H).

### Preparation of Compound 126

### Step 1

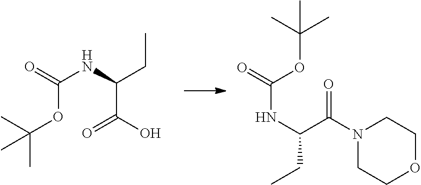 [SMILES: CC[C@H](NC(=O)OC(C)(C)C)C(=O)O ; CC[C@H](NC(=O)OC(C)(C)C)C(=O)N1CCOCC1]

### Tert-butyl N-[(1S)-1-(morpholine-4-carbonyl)propyl]carbamate

To a solution of (S)-2-(tert-butoxycarbonylamino)butanoic acid (18.5 g, 91.1 mmol), HBTU (43.4 g, 109 mmol), DIPEA (47.6 mL, 273 mmol) in CH₂Cl₂ (400 mL) at 0° C., morpholine (9.5 mL, 110 mmol) was added and the reaction stirred at RT for 16 h. Reaction progress was monitored by TLC. The mixture was diluted with CH₂Cl₂ (200 mL), washed with water (3×100 mL), brine (100 mL), dried over Na₂SO₄ and concentrated. The residue was purified by column chromatography (100-200 mesh silica gel, 50% EtOAc in petroleum ether) to afford tert-butyl N-[(1S)-1-(morpholine-4-carbonyl)propyl]carbamate (22 g, 89%, colorless gum). TLC system: 50% EtOAc: petroleum ether, Rf: 0.53. MH⁺=273.0. ¹H NMR (CDCl₃, 400 MHz): 5.38 (d, J=7.6 Hz; 1H), 4.55-4.50 (m, 1H), 3.71 (m, 8H), 1.77-1.71 (m, 1H), 1.56-1.51 (m, 1H), 1.43 (m, 9H).

### Step 2

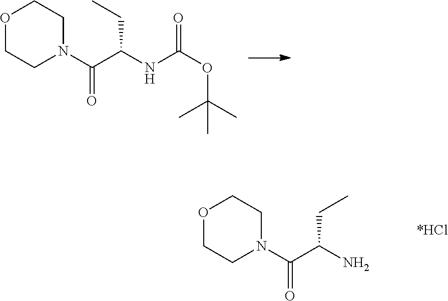 [SMILES: CC[C@H](NC(=O)OC(C)(C)C)C(=O)N1CCOCC1 ; CC[C@H](N)C(=O)N1CCOCC1]

### (2S)-2-amino-1-morpholino-butan-1-one hydrochloride

To a solution of tert-butyl N-[(1S)-1-(morpholine-4-carbonyl)propyl]carbamate (23 g, 84 mmol) in CH₂Cl₂ (100 mL) was added a solution of 2M HCl in Et₂O (100 mL) at 0° C. and stirred at RT for 16 h. Reaction progress was monitored by TLC. Upon completion, the reaction mixture was concentrated, the residue was washed with Et₂O (2×20 mL), filtered and dried to afford (2S)-2-amino-1-morpholino-butan-1-one hydrochloride. TLC: 50% EtOAc: petroleum ether, R_{f}: 0.05. MH⁺=173.2. ¹H NMR (DMSO-d6, 400 MHz): 8.37 (bs, exchanged with D₂O, 2H), 4.33 (d, J=5.2 Hz; 1H), 3.63-3.40 (m, 8H), 1.82-1.65 (m, 2H), 1.91 (t, J=7.47 Hz; 3H).

### Step 3

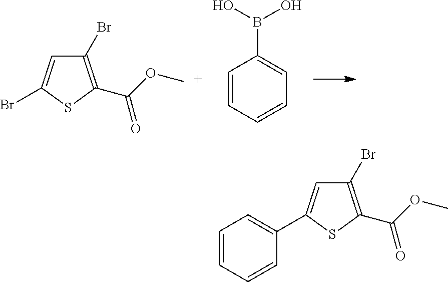

### Methyl 3-bromo-5-phenyl-thiophene-2-carboxylate

To a solution of methyl 3,5-dibromothiophene carboxylate (2.0 g, 6.7 mmol) and phenylboronic acid (812 mg, 6.66 mmol) in toluene (60 mL), 3M aq. K₂CO₃ solution (6.63 mL) was added and the reaction mixture was deoxygenated by bubbling with argon for 1 h. Pd(PPh₃)₄ was added, argon bubbling was continued for another 30 min, and the mixture stirred at 90° C. for 3 h. Reaction progress was monitored by TLC. Upon completion the reaction mixture was diluted with EtOAc (100 mL), washed with water (3×30 mL), brine solution (30 mL), dried over Na₂SO₄, filtered and concentrated. The residue was purified by column chromatography (100-200 mesh silica gel, 5% EtOAc/petroleum ether) to afford methyl 3-bromo-5-phenyl-thiophene-2-carboxylate (1.5 g, 75%, white solid. TLC: 5% EtOAc in petroleum ether, R_{f}: 0.42. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=297.0, t_{R}=4.86 min. ¹H NMR (400 MHz, CDCl₃): 7.61-7.59 (m, 2H), 7.45-7.39 (m, 3H), 7.29 (s, 1H), 3.91 (s, 3H).

### Step 4

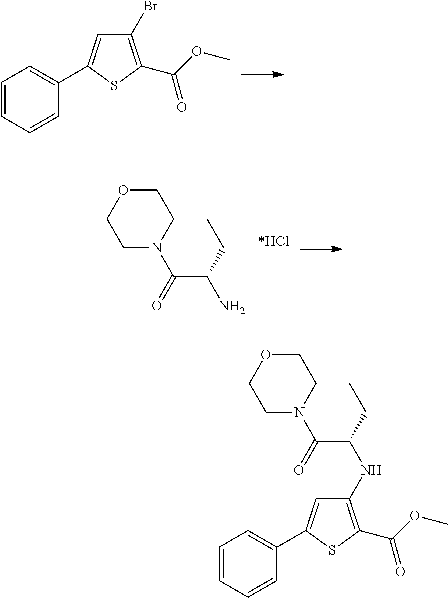 [SMILES: COC(=O)c1sc(-c2ccccc2)cc1Br ; CC[C@H](N)C(=O)N1CCOCC1 ; CC[C@H](Nc1cc(-c2ccccc2)sc1C(=O)OC)C(=O)N1CCOCC1]

### Methyl 3-[[(1S)-1-(morpholine-4-carbonyl)propyl]amino]-5-phenyl-thiophene-2-carboxylate

A suspension of methyl 3-bromo-5-phenyl-thiophene-2-carboxylate (500 mg, 1.70 mmol), (S)-2-amino-1-butan-1-one.HCl (530 mg, 2.54 mmol), Cs₂CO₃ (1.65 g, 5.08 mmol) in toluene (30 mL) was deoxygenated by purging a stream of argon for 60 min, and Pd(OAc)₂ (38 mg, 0.17 mmol), (±) BINAP (106 mg, 0.17 mmol) were added. The purging was continued for another 30 min and the mixture was stirred at 100° C. for 16 h. Reaction progress was monitored by TLC. Upon completion the reaction mixture was cooled to RT, diluted with EtOAc (100 mL) and filtered through celite. The filtrate was washed with water (2×35 mL), brine (30 mL), dried over Na₂SO₄, and concentrated. The residue was purified by column chromatography (100-200 mesh silica gel, 30% EtOAc in petroleum ether) to afford methyl 3-[[(1S)-1-(morpholine-4-carbonyl)propyl]amino]-5-phenyl-thiophene-2-carboxylate (400 mg, 40%, white solid). TLC: 50% EtOAc in petroleum ether. R_{f}: 0.36. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=389.2, t_{R}=4.33 min. ¹H NMR (400 MHz, DMSO-d6): 7.73-7.71 (m, 2H), 7.49-7.40 (m, 4H), 4.88-4.86 (m, 1H), 3.75 (s, 3H), 3.63-3.55 (m, 6H), 3.46-3.42 (m, 2H), 1.77-1.75 (m, 1H), 1.60-1.57 (m, 1H), 0.84 (t, J=7.6 Hz; 3H).

### Step 5

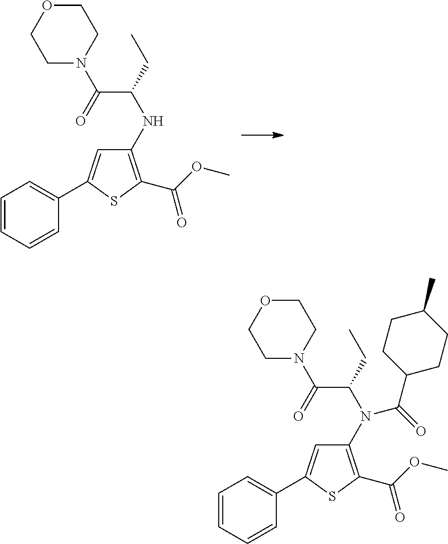

### Methyl 3-[(trans-4-methylcyclohexanecarbonyl)-[(1S)-1-(morpholine-4-carbonyl)propyl]amino]-5-phenyl-thiophene-2-carboxylate

To a stirred solution of DCE (20 mL) containing methyl 3-[[(1S)-1-(morpholine-4-carbonyl)propyl]amino]-5-phenyl-thiophene-2-carboxylate (300 mg, 0.773 mmol), pyridine (3 mL, 10 vol), and DMAP (47 mg, 0.39 mmol) were added followed by dropwise addition of a solution of trans-4-methylcyclohexylcarbonyl chloride (1.24 g, 7.73 mmol) in DCE (5 mL) at 0° C. After addition, the reaction mixture was stirred at 100° C. for 16 h. Reaction progress was monitored by TLC. Upon completion, the reaction mixture was diluted with EtOAc (80 mL), washed with 2N aq. HCl (2×30 mL), water (30 mL), 10% NaHCO₃ solution (2×30 mL), brine (30 mL), dried over Na₂SO₄ and concentrated. The residue was purified by column chromatography (100-200 mesh silica gel, 50% EtOAc in petroleum ether) to afford methyl 3-[(trans-4-methylcyclohexanecarbonyl)-[(1S)-1-(morpholine-4-carbonyl)propyl]amino]-5-phenyl-thiophene-2-carboxylate (220 mg, 56%, white solid). TLC: 40% EtOAc in petroleum ether. R_{f}: 0.38. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=513.1, t_{R}=4.89 min. ¹H NMR (400 MHz, DMSO-d6): 7.82-7.74 (m, 2H), 7.56-7.44 (m, 4H), 5.59 (br s, 1H), 5.30-5.26 (m, 1H), 3.80-3.44 (m, 11H), 2.04-1.91 (m, 2H), 1.70-1.16 (m, 8H), 0.95-0.52 (m, 8H).

### Step 6

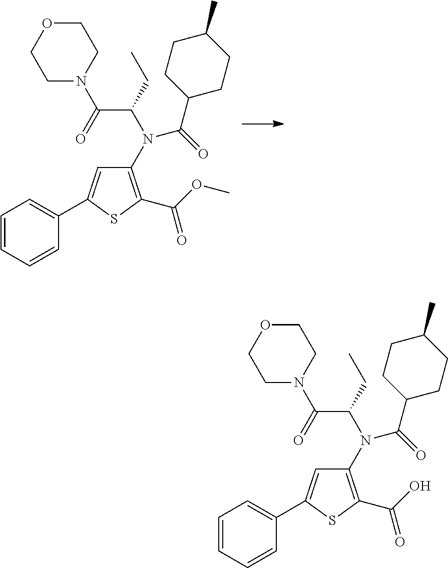

### 3-[(trans-4-methylcyclohexanecarbonyl)-[(1S)-1-(morpholine-4-carbonyl)propyl]amino]-5-phenyl-thiophene-2-carboxylic acid

To a stirred solution of methyl 3-[(trans-4-methylcyclohexanecarbonyl)-[(1S)-1-(morpholine-4-carbonyl)propyl]amino]-5-phenyl-thiophene-2-carboxylate (220 mg, 0.429 mmol) in 1:1 mixture of THF and water (4 mL), LiOH.H₂O (54 mg, 1.3 mmol) was added at RT and the mixture stirred for 16 h. Reaction progress was monitored by TLC. Upon completion, the reaction was acidified (pH˜1) with 1M aq. HCl and extracted with EtOAc (60 mL). The organic layer was washed with water (3×20 mL), brine (20 mL), dried over Na₂SO₄ and concentrated. The resulting residue was purified by trituration with Et₂O (2×5 mL) followed by filtration to afford 126, 3-[(trans-4-methylcyclohexanecarbonyl)-[(1S)-1-(morpholine-4-carbonyl)propyl]amino]-5-phenyl-thiophene-2-carboxylic acid (120 mg, 56%, white solid). TLC: 10% MeOH in CHCl₃. R_{f}: 0.33. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=499.0, MH⁻=497.0, t_{R}=4.29 min. ¹H NMR (400 MHz, DMSO-d6): 13.4 (br s, exchanged with D₂O; 2H), 7.80-7.71 (m, 2H), 7.50-7.43 (m, 4H), 5.61 (br s, 1H), 5.29-5.25 (m, 1H), 3.72-3.40 (m, 8H), 2.11-2.05 (m, 1H), 1.85-1.82 (m, 1H), 1.71-1.40 (m, 6H), 1.32-1.15 (m, 2H), 0.91-0.81 (m, 3H), 0.77-0.5 (m).

### Preparation of Compound 127

### Step 1

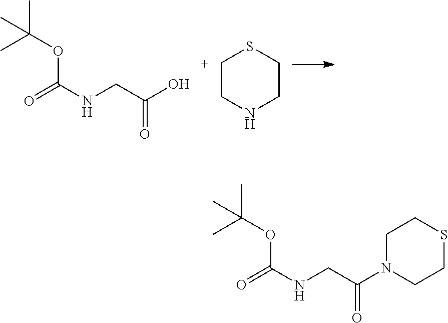

### Tert-butyl N-(2-oxo-2-thiomorpholino-ethyl)carbamate

To a solution of N-Boc glycine (2 g, 12 mmol), HBTU (9.1 g, 23 mmol), and DIPEA (6 mL, 34 mmol) in CH₂Cl₂ (50 mL) at 0° C., thiomorpholine (1.64 mL, 17.2 mmol) was added and the mixture stirred at RT for 16 h. Reaction progress was monitored by TLC. Upon completion the reaction mixture was diluted with CH₂Cl₂ (100 mL), washed with water (3×50 mL), brine (30 mL), dried over Na₂SO₄ and concentrated. The crude product was purified by column chromatography (100-200 mesh silica gel, 50% EtOAc in petroleum ether) to afford tert-butyl N-(2-oxo-2-thiomorpholino-ethyl)carbamate (2.9 g, 54%, white solid). TLC: 97% EtOAc: petroleum ether. R_{f}: 0.48. Analysis by LCMS (column: BEH C-18 (2.1×50 mm, 1.7 μm), mobile phase: A: 0.025% aq. TFA, B: 0.025% TFA/MeCN, time (min)/% B gradient: 0 min/15%, 3/95, 4/95, 4.1/15, flow rate: 0.4 mL/min, sample diluent: MeCN): MH⁺=261.2, t_{R}=1.62 min. ¹H NMR (400 MHz, CDCl₃): 5.51 (br s, 1H), 3.95-3.88 (m, 4H), 2.80 (s, 2H), 2.64-2.61 (m, 4H), 1.45 (s, 9H).

### Step 2

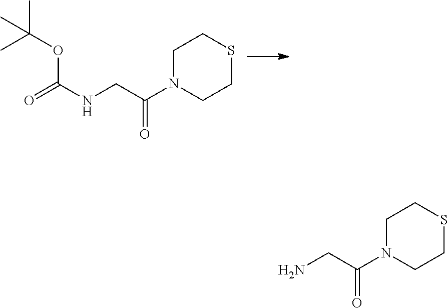 [SMILES: CC(C)(C)OC(=O)NCC(=O)N1CCSCC1 ; NCC(=O)N1CCSCC1]

### 2-amino-1-thiomorpholino-ethanone

To a solution of tert-butyl N-(2-oxo-2-thiomorpholino-ethyl)carbamate (2.9 g, 11 mmol) in CH₂Cl₂ (50 mL) at 0° C. was added 2M HCl in Et₂O (50 mL), and the mixture stirred at RT for 16 h. Reaction progress was monitored by TLC. Upon completion the reaction mixture was concentrated and the resulting residue triturated with Et₂O (3×10 mL), filtered and dried to afford 2-amino-1-thiomorpholino-ethanone (1.5 g, 68%, white solid). MH⁺=161.1. ¹H NMR (400 MHz, DMSO-d6): 8.33 (br s, exchanged with D₂O; 2H), 3.86-3.85 (m, 2H), 3.76-3.74 (m, 2H), 3.63-3.60 (m, 2H), 2.67-2.64 (m, 2H), 2.58-2.55 (m, 2H).

### Step 3

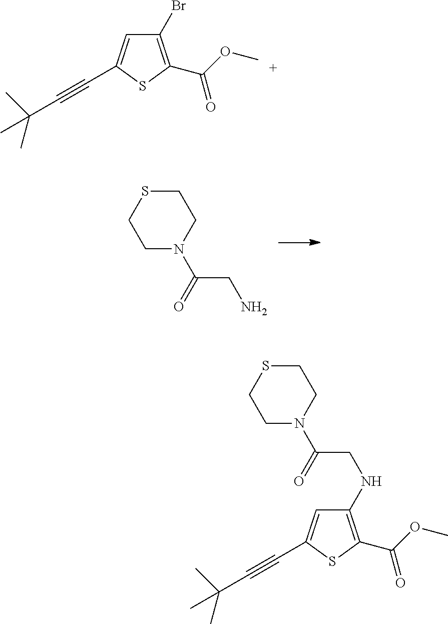 [SMILES: COC(=O)c1sc(C#CC(C)(C)C)cc1Br ; NCC(=O)N1CCSCC1 ; COC(=O)c1sc(C#CC(C)(C)C)cc1NCC(=O)N1CCSCC1]

### Methyl 5-(3,3-dimethylbut-1-ynyl)-3-[(2-oxo-2-thiomorpholino-ethyl)amino]thiophene-2-carboxylate

A suspension of methyl 3-bromo-5-(3,3-dimethylbut-1-ynyl)thiophene-2-carboxylate (500 mg, 1.66 mmol), 2-amino-1-thiomorpholinoethanone.HCl (487 mg, 2.49 mmol), and Cs₂CO₃ (1.62 g, 4.98 mmol) in toluene (30 mL) was deoxygenated by purging a stream of argon for 60 min, then Pd(OAc)₂ (37.3 mg, 0.160 mmol) and (±) BINAP (103 mg, 0.160 mmol) were added. Purging was continued for another 30 min, then the mixture stirred at 100° C. for 16 h. Reaction progress was monitored by TLC. The reaction mixture was cooled to RT, diluted with EtOAc (100 mL) and filtered through celite. The filtrate was washed with water (2×35 mL), brine (30 mL), dried over Na₂SO₄, and concentrated. The residue was purified by column chromatography (100-200 mesh silica gel, 30% EtOAc in petroleum ether) to afford methyl 5-(3,3-dimethylbut-1-ynyl)-3-[(2-oxo-2-thiomorpholino-ethyl)amino]thiophene-2-carboxylate (180 mg, 28%, white solid). TLC: 50% EtOAc in petroleum ether. R_{f}: 0.53. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=381.0, t_{R}=4.71 min. ¹H NMR (400 MHz, DMSO-d6): 7.26 (br s, exchanged with D₂O; 1H), 6.99 (s, 1H), 4.14 (d, J=4.8 Hz; 2H), 3.75-3.69 (m, 7H), 2.65-2.57 (m, 4H), 1.28 (s, 9H).

### Step 4

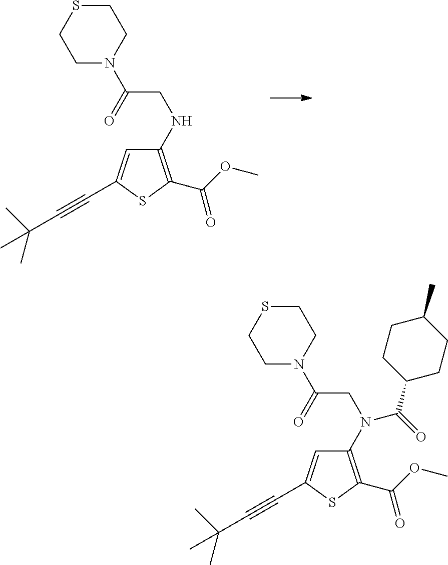 [SMILES: COC(=O)c1sc(C#CC(C)(C)C)cc1NCC(=O)N1CCSCC1 ; COC(=O)c1sc(C#CC(C)(C)C)cc1N(CC(=O)N1CCSCC1)C(=O)[C@H]1CC[C@H](C)CC1]

### Methyl 5-(3,3-dimethylbut-1-ynyl)-3-[(trans-4-methylcyclohexanecarbonyl)-(2-oxo-2-thiomorpholino-ethyl)amino]thiophene-2-carboxylate

A solution of methyl 5-(3,3-dimethylbut-1-ynyl)-3-[(2-oxo-2-thiomorpholino-ethyl)amino]thiophene-2-carboxylate (180 mg, 0.473 mmol) in DCE (10 mL) was stirred with pyridine (2 mL, 10 vol) and DMAP (29 mg, 0.24 mmol). A solution of trans-4-methylcyclohexylcarbonyl chloride (760 mg, 4.74 mmol) in DCE (5 mL) was then added dropwise at 0° C. After addition, the reaction mixture was stirred at 100° C. for 16 h. Reaction progress was monitored by TLC. Upon completion, the reaction mixture was diluted with EtOAc (80 mL), washed with 2N aq. HCl (2×30 mL), water (30 mL), 10% NaHCO₃ solution (2×30 mL), brine (30 mL), dried over Na₂SO₄ and concentrated. The residue was purified by column chromatography (100-200 mesh silica gel, 40% EtOAc in petroleum ether) to afford methyl 5-(3,3-dimethylbut-1-ynyl)-3-[(trans-4-methylcyclohexanecarbonyl)-(2-oxo-2-thiomorpholino-ethyl)amino]thiophene-2-carboxylate (200 mg, 84%, white solid). TLC: 40% EtOAc in petroleum ether. R_{f}: 0.53. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=505.1, t_{R}=5.38 min. ¹H NMR (400 MHz, DMSO-d6): 7.24 (s, 1H), 4.90 (d, J=16.8 Hz; 1H), 3.84 (d, J=16.4 Hz; 1H), 3.77 (s, 3H), 3.74-3.62 (m, 4H), 2.60-2.50 (m, 4H), 2.11-2.05 (m, 1H), 1.71-1.47 (m, 5H), 1.40-1.23 (m, 11H), 0.77 (d, J=6 Hz; 3H), 0.67-0.64 (m, 2H).

### Step 5

### 5-(3,3-dimethylbut-1-ynyl)-3-[(trans-4-methylcyclohexanecarbonyl)-(2-oxo-2-thiomorpholino-ethyl)amino]thiophene-2-carboxylic acid

To a stirred solution of methyl 5-(3,3-dimethylbut-1-ynyl)-3-[(trans-4-methylcyclohexanecarbonyl)-(2-oxo-2-thiomorpholino-ethyl)amino]thiophene-2-carboxylate (180 mg, 0.357 mmol) in 1:1 THF/water (4 mL) was added LiOH.H₂O (45 mg, 1.1 mmol) and the mixture stirred for 16 h. Reaction progress was monitored by TLC. Upon completion, the reaction mixture was acidified (pH˜1) with 1M aq. HCl and extracted with EtOAc (60 mL). The organic layer was washed with water (3×20 mL), brine (20 mL), dried over Na₂SO₄, and concentrated. The residue was purified by prep TLC (5% MeOH/CHCl₃) to afford 127, 5-(3,3-dimethylbut-1-ynyl)-3-[(trans-4-methylcyclohexanecarbonyl)-(2-oxo-2-thiomorpholino-ethyl)amino]thiophene-2-carboxylic acid (135 mg, 77%, white solid). TLC: 10% MeOH in CHCl₃, R_{f}: 0.35. Analysis by LCMS (column: Xterra MS-C-18 (4.6×50 mm, 3 μm), mobile phase: A: 0.1% aq. HCOOH, B: MeCN, time (min)/% B gradient: 0 min/10%, 4/90, 8/90, 8.01/10, flow rate: 1.0 mL/min, sample diluent: MeCN): MH⁺=491.2, t_{R}=4.85 min. ¹H NMR (400 MHz, DMSO-d6): 13.45 (br s, exchanged with D₂O; 1H), 7.21 (s, 1H), 4.90 (d, J=16.8 Hz; 1H), 3.84 (d, J=14.4 Hz; 1H), 3.72-3.63 (m, 4H), 2.64-2.59 (m, 4H), 2.12-2.06 (m, 2H), 1.69-1.53 (m, 4H), 1.40-1.33 (m, 11H), 0.77 (d, J=6.4 Hz; 3H), 0.70-0.61 (m, 2H).

### Preparation of Compound 128

### Step 1

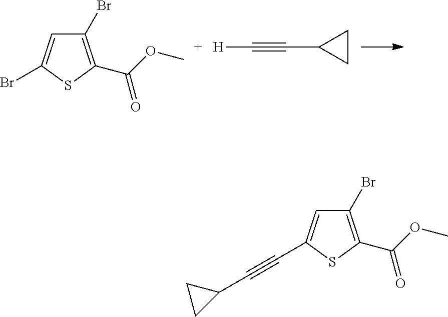

### Methyl 3-bromo-5-(2-cyclopropylethynyl)thiophene-2-carboxylate

A suspension of CuI (380 mg, 1.99 mmol) in 1, 4 dioxane (30 mL) was deoxygenated by purging with argon for 30 min at RT, then Pd(PPh₃)₂Cl₂ (1.4 g, 2.0 mmol) added. Purging was continued for 15 min, then diisopropylamine was (1.9 mL, 13 mmol) added followed by methyl-3,5-dibromothiophene-2-carboxylate (2.0 g, 6.7 mmol). The reaction mixture was stirred for 15 min at RT, then cooled in ice-water and cyclopropylacetylene (0.56 mL, 6.7 mmol) in dioxane (10 mL) added at 5-10° C. After addition the reaction mixture was stirred at RT for 1 h. Reaction progress was monitored by TLC. Upon completion, the reaction mixture was diluted with EtOAc (20 mL), filtered through celite, and the filter cake washed with EtOAc (2×10 mL). The combined filtrate was concentrated and the resulting crude product purified by column chromatography (100-200 mesh silica gel, 2% EtOAc in petroleum ether as eluent) to afford methyl 3-bromo-5-(2-cyclopropylethynyl)thiophene-2-carboxylate (1.45 g, 76%, yellow liquid). TLC: 2% EtOAc in petroleum ether, R_{f}: 0.5. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=285.1, t_{R}=2.05 min. ¹H NMR (400 MHz, CDCl₃): 7.02 (s, 1H), 3.86 (s, 3H), 1.49-1.46 (m, 1H), 0.96-0.83 (m, 4H).

### Step 2

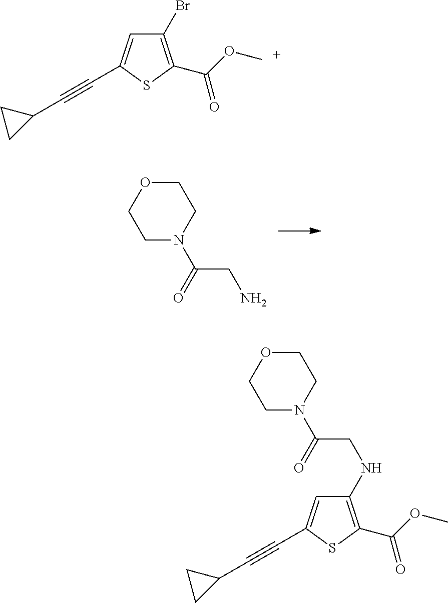

### Methyl 5-(2-cyclopropylethynyl)-3-[(2-morpholino-2-oxo-ethyl)amino]thiophene-2-carboxylate

A suspension of methyl 3-bromo-5-(2-cyclopropylethynyl)thiophene-2-carboxylate (350 mg, 1.23 mmol), 2-amino-1-morpholino ethanone.HCl (332 mg, 1.84 mmol), and Cs₂CO₃ (1.2 g, 3.7 mmol) in toluene (10 mL) was deoxygenated by bubbling with argon for 50 min, then Pd(OAc)₂ (28 mg, 0.12 mmol) and (±) BINAP (76 mg, 0.12 mmol) were added. The purging was continued for another 10 min and the mixture stirred at 100° C. for 16 h. Reaction progress was monitored by TLC. Upon completion, the mixture was cooled to RT, diluted with EtOAc (50 mL), and filtered through celite. The filtrate was washed with water (2×20 mL), brine (30 mL), dried over Na₂SO₄, and concentrated. The resulting residue was purified (column chromatography, 100-200 mesh silica gel, 40% EtOAc in petroleum ether as eluent) to afford methyl 5-(2-cyclopropylethynyl)-3-[(2-morpholino-2-oxo-ethyl)amino]thiophene-2-carboxylate (50 mg, 12%, yellow solid). TLC: 60% EtOAc in petroleum ether, R_{f}: 0.35. MH⁺=349.2. ¹H NMR (400 MHz, DMSO-d6): 7.32 (br s, exchanged with D₂O; 1H), 6.53 (s, 1H), 3.99 (d, J=4.8 Hz; 2H), 3.81 (s, 3H), 3.70-3.67 (m, 6H), 3.44-3.42 (m, 2H), 1.49-1.41 (m, 1H), 0.92-0.88 (m, 2H), 0.84-0.81 (m, 2H).

### Step 3

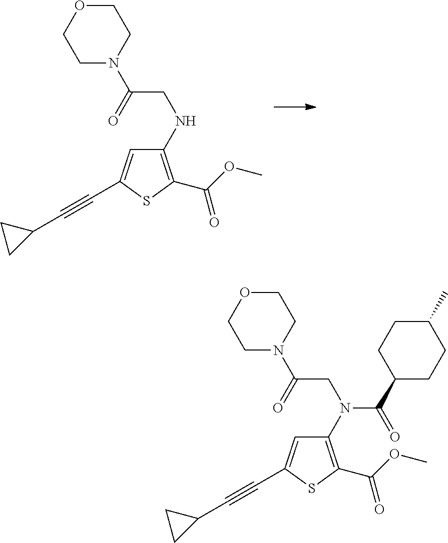 [SMILES: COC(=O)c1sc(C#CC2CC2)cc1NCC(=O)N1CCOCC1 ; COC(=O)c1sc(C#CC2CC2)cc1N(CC(=O)N1CCOCC1)C(=O)[C@H]1CC[C@H](C)CC1]

### Methyl 5-(2-cyclopropylethynyl)-3-[(trans-4-methylcyclohexanecarbonyl)-(2-morpholino-2-oxo-ethyl)amino]thiophene-2-carboxylate

To a 0° C. solution of methyl 5-(2-cyclopropylethynyl)-3-[(2-morpholino-2-oxo-ethyl)amino]thiophene-2-carboxylate (50 mg, 0.14 mmol) and Et₃N (0.2 mL) in CH₂Cl₂ (5.0 mL) was added dropwise a solution of trans 4-methylcyclohexyl carbonyl chloride (230 mg, 1.43 mmol) in CH₂Cl₂ (5.0 mL), then the reaction stirred at 90° C. for 16 h. Reaction progress was monitored by TLC. Upon completion, the reaction mixture was diluted with EtOAc (50 mL), washed with 2N aq. HCl (2×10 mL), water (10 mL), 10% NaHCO₃ solution (20 mL), brine (20 mL), dried over Na₂SO₄, and concentrated. The residue was purified by column chromatography (100-200 mesh silica gel, 50% EtOAc in petroleum ether as eluent) to afford methyl 5-(2-cyclopropylethynyl)-3-[(trans-4-methylcyclohexanecarbonyl)-(2-morpholino-2-oxo-ethyl)amino]thiophene-2-carboxylate (22 mg, 30%, yellow solid). TLC: 60% EtOAc in petroleum ether, R_{f}: 0.35. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 3/100, 4/100, flow rate: 0.4 mL/min, sample diluent: MeOH): MH⁺=473.4. t_{R}=2.02 min. ¹H NMR (400 MHz, DMSO-d6): 7.21 (s, 1H), 4.91 (d, J=16.8 Hz; 1H), 3.84 (d, J=16.8 Hz; 1H), 3.77 (s, 3H), 3.54 (br s, 4H), 3.43-3.31 (m, 4H), 2.09-2.03 (m, 1H), 1.66-1.46 (m, 5H), 1.40-1.23 (m, 4H), 0.97-0.92 (m, 2H), 0.87-0.80 (m, 2H), 0.77 (d, J=6.4 Hz; 3H), 0.6.

### Step 4

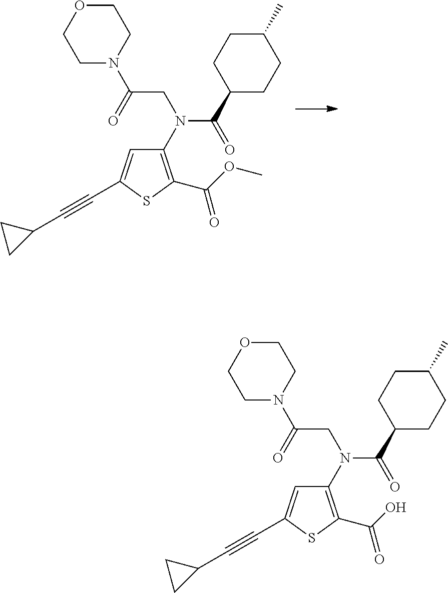 [SMILES: COC(=O)c1sc(C#CC2CC2)cc1N(CC(=O)N1CCOCC1)C(=O)[C@H]1CC[C@H](C)CC1 ; C[C@H]1CC[C@H](C(=O)N(CC(=O)N2CCOCC2)c2cc(C#CC3CC3)sc2C(=O)O)CC1]

### 5-(2-cyclopropylethynyl)-3-[(trans-4-methylcyclohexanecarb onyl)-(2-morpholino-2-oxo-ethyl)amino]thiophene-2-carboxylic acid

To a RT stirred solution of methyl 5-(2-cyclopropylethynyl)-3-[(trans-4-methylcyclohexanecarbonyl)-(2-morpholino-2-oxo-ethyl)amino]thiophene-2-carboxylate (20 mg, 0.042 mmol) in 1:1 THF/water (6 mL), LiOH.H₂O (5.3 mg, 0.62 mmol) was added and the reaction stirred for 16 h. Reaction progress was monitored by TLC. Upon completion, the mixture was acidified (pH˜1) with 1M aq. HCl and extracted with EtOAc (50 mL). The organic layer was washed with water (2×10 mL), brine (10 mL), dried over Na₂SO₄ and concentrated to afford 128, 5-(2-cyclopropylethynyl)-3-[(trans-4-methylcyclohexanecarbonyl)-(2-morpholino-2-oxo-ethyl)amino]thiophene-2-carboxylic acid (8 mg, 39%, white solid). TLC: 10% MeOH in CHCl₃, R_{f}: 0.3. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.8/50, flow rate: 0.4 mL/min, sample diluent: MeOH): MH⁺=457.5. t_{R}=1.34 min. ¹H NMR (400 MHz, DMSO-d6): 13.45 (br s, exchanged with D₂O; 1H), 4.91 (d, J=16.4 Hz; 1H), 3.84 (d, J=16.4 Hz; 1H), 3.54-3.53 (m, 4H), 3.39-3.31 (m, 4H), 2.10-2.05 (m, 1H), 1.68-1.48 (m, 5H), 1.40-1.23 (m, 4H), 0.97-0.92 (m, 2H), 0.88-0.79 (m, 2H), 0.77 (d, J=6.4).

### Preparation of Compound 129

### Step 1

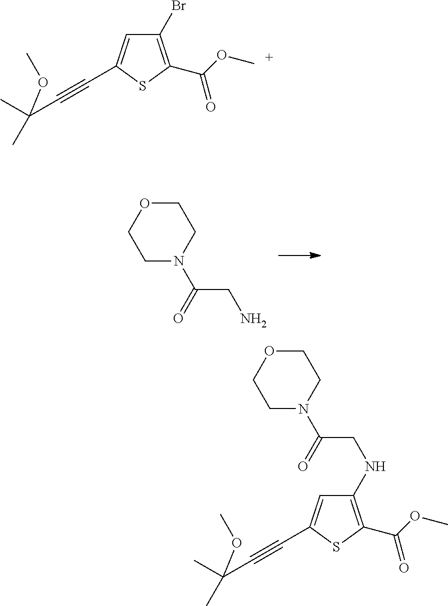

### Methyl 5-(3-methoxy-3-methyl-but-1-ynyl)-3-[(2-morpholino-2-oxo-ethyl)amino]thiophene-2-carboxylate

A suspension of methyl 3-bromo-5-(3-methoxy-3-methylbut-1-ynyl)thiophene-2-carboxylate (prepared as precedented, 1.6 g, 5.0 mmol), 2-amino-1-morpholinoethanone.HCl (1.48 g, 7.57 mmol), and Cs₂CO₃ (4.93 g, 15.1 mmol) in toluene (10 mL) was deoxygenated by bubbling with argon for 50 min, then Pd(OAc)₂ (113 mg, 0.504 mmol) and (±) BINAP (314 mg, 0.504 mmol) were added. Purging was continued for another 10 min and the mixture stirred at 100° C. for 16 h. Reaction progress was monitored by TLC. Upon completion, the reaction mixture was cooled to RT, diluted with EtOAc (100 mL) and filtered through celite. The filtrate was washed with water (2×50 mL), brine (30 mL), dried over Na₂SO₄, and concentrated. The residue was purified (column chromatography, 100-200 mesh silica gel, 60% EtOAc in petroleum ether as eluent) to afford methyl 5-(3-methoxy-3-methyl-but-1-ynyl)-3-[(2-morpholino-2-oxo-ethyl)amino]thiophene-2-carboxylate (700 mg, 37%, yellow solid). TLC: 50% EtOAc in petroleum ether, R_{f}: 0.35. Analysis by LCMS (column: Xterra RP-18 (4.6×100 mm, 5 μm), mobile phase: A: 0.1% aq. CH₃CO₂NH₄, B: MeCN, time (min)/% B gradient: 0 min/10%, 4/70, 6/95, 14/95, 15/10, flow rate: 0.8 mL/min, sample diluent: MeCN): MH⁺=379.2. ¹H NMR (400 MHz, DMSO-d6): 7.27-7.25 (m, exchanged with D₂O; 1H), 7.09 (s, 1H), 4.16 (d, J=4.8 Hz; 2H), 3.73 (s, 3H), 3.62-3.56 (m, 4H), 3.49-3.44 (m, 4H), 3.28 (s, 3H), 1.46 (s, 6H).

### Step 2

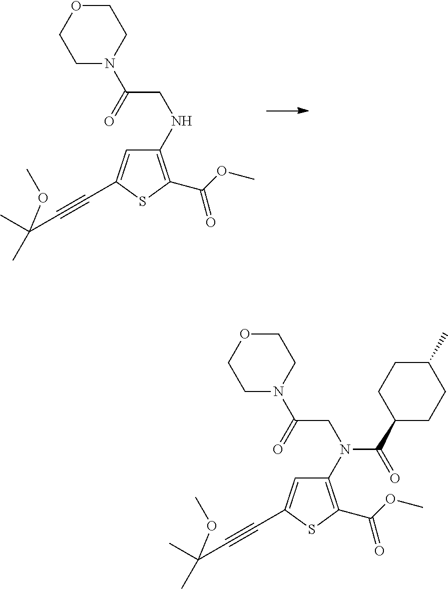 [SMILES: COC(=O)c1sc(C#CC(C)(C)OC)cc1NCC(=O)N1CCOCC1 ; COC(=O)c1sc(C#CC(C)(C)OC)cc1N(CC(=O)N1CCOCC1)C(=O)[C@H]1CC[C@H](C)CC1]

### Methyl 5-(3-methoxy-3-methyl-but-1-ynyl)-3-[(trans-4-methylcyclohexanecarbonyl)-(2-morpholino-2-oxo-ethyl)amino]thiophene-2-carboxylate

To a solution of methyl 5-(3-methoxy-3-methyl-but-1-ynyl)-3-[(2-morpholino-2-oxo-ethyl)amino]thiophene-2-carboxylate (300 mg, 0.79 mmol), pyridine, (4.0 mL), and DMAP (49 mg, 0.40 mmol) in DCE (5 mL) at 0° C. was added dropwise a solution of trans 4-methylcyclohexyl carbonyl chloride (1.3 g, 7.3 mmol) in DCE (5 mL). The solution was then stirred at 90° C. for 16 h. Reaction progress was monitored by TLC. Upon completion, the reaction mixture was diluted with EtOAc (80 mL), washed with 2N aq. HCl (2×10 mL), water (10 mL), 10% NaHCO₃ solution (2×20 mL), brine (20 mL), dried over Na₂SO₄, and concentrated. The residue was purified (column chromatography, 100-200 mesh silica gel, 50% EtOAc in petroleum ether as eluent) to afford methyl 5-(3-methoxy-3-methyl-but-1-ynyl)-3-[(trans-4-methylcyclohexanecarbonyl)-(2-morpholino-2-oxo-ethyl)amino]thiophene-2-carboxylate (301 mg, 75%, white solid). TLC: 50% EtOAc in petroleum ether, R_{f}: 0.3. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=505.5. t_{R}=1.92 min. ¹H NMR (400 MHz, DMSO-d6): 7.36 (s, 1H), 4.92 (d, J=16.8 Hz; 1H), 3.87 (d, J=16.8 Hz; 1H), 3.78 (s, 3H), 3.54 (m, 4H), 3.41-3.39 (m, 4H), 3.29 (s, 3H), 2.11 (m, 1H), 1.67-1.55 (m, 3H), 1.48 (m, 6H), 1.42-1.23 (m, 4H), 0.77 (d, J=6.4 Hz; 3H), 0.71-0.62 (m, 2H).

### Step 3

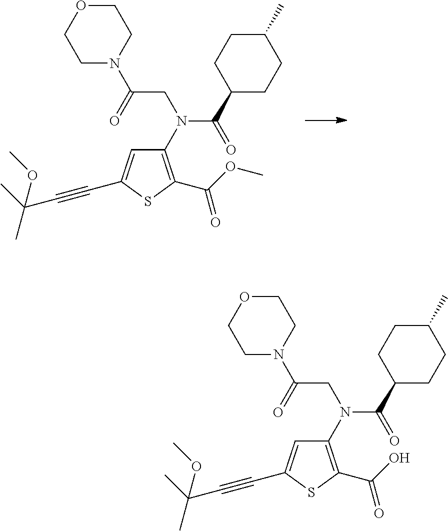

### 5-(3-methoxy-3-methyl-but-1-ynyl)-3-[(trans-4-methylcyclohexanecarbonyl)-(2-morpholino-2-oxo-ethyl)amino]thiophene-2-carboxylic acid

To a stirred RT solution of methyl 5-(3-methoxy-3-methyl-but-1-ynyl)-3-[(trans-4-methylcyclohexanecarbonyl)-(2-morpholino-2-oxo-ethyl)amino]thiophene-2-carboxylate (180 mg, 0.357 mmol) in 1:1 THF/water (4 mL) was added LiOH.H₂O (45 mg, 1.1 mmol); the mixture was then stirred for 16 h. Reaction progress was monitored by TLC. Upon completion, the reaction was acidified (pH˜1) with 1M aq. HCl and extracted with EtOAc (80 mL). The organic layer was washed with water (2×10 mL), brine (10 mL), dried over Na₂SO₄, and concentrated to afford 129, 5-(3-methoxy-3-methyl-but-1-ynyl)-3-[(trans-4-methylcyclohexanecarbonyl)-(2-morpholino-2-oxo-ethyl)amino]thiophene-2-carboxylic acid (125 mg, 72%, white solid). TLC: 10% MeOH in CHCl₃, R_{f}: 0.4. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=491.5. t_{R}=1.3 min. ¹H NMR (400 MHz, DMSO-d6): 13.62 (br s, exchanged with D₂O; 1H), 7.33 (s, 1H), 4.92 (d, J=16.4 Hz; 1H), 3.87 (d, J=16.8 Hz; 1H), 3.54-3.40 (m, 8H), 3.29 (s, 3H), 2.09 (m, 1H), 1.65-1.52 (m, 4H), 1.48 (s, 6H), 1.39-1.23 (m, 4H), 0.77 (d, J=6.4 Hz; 3H), 0.68-0.65.

### Preparation of Compound 130

### Step 1

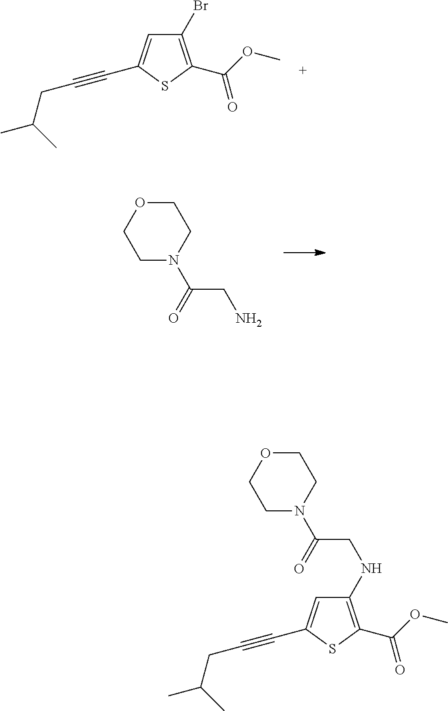

### Methyl 5-(4-methylpent-1-ynyl)-3-[(2-morpholino-2-oxo-ethyl)amino]thiophene-2-carboxylate

A suspension of crude methyl 3-bromo-5-(4-methylpent-1-ynyl)thiophene-2-carboxylate (prepared as precedented, 1.55 g, 5.16 mmol), 2-amino-1-butan-1-one.HCl (1.39 g, 7.75 mmol), and Cs₂CO₃ (5.05 g, 15.5 mmol) in toluene (35 mL) was deoxygenated by purging a stream of argon for 30 min, then Pd(OAc)₂ (116 mg, 0.510 mmol) and (±) BINAP (322 mg, 0.520 mmol) added. The purging was continued for another 30 min and the mixture was stirred at 100° C. for 16 h. Reaction progress was monitored by TLC. Upon completion the reaction mixture was cooled to RT, diluted with EtOAc (100 mL) and filtered through celite. The filtrate was washed with water (2×20 mL), brine (30 mL), dried over Na₂SO₄, and concentrated. The residue was purified (column chromatography, 100-200 mesh silica gel, 30% EtOAc in petroleum ether as eluent) to afford methyl 5-(4-methylpent-1-ynyl)-3-[(2-morpholino-2-oxo-ethyl)amino]thiophene-2-carboxylate (120 mg, white solid). TLC: 40% EtOAc in petroleum ether, R_{f}: 0.5. MH⁺=365.2. ¹H NMR (400 MHz, DMSO-d6): 7.2 (br s, exchanged with D₂O; 1H), 6.99 (s, 1H), 4.15 (d, J=4.4 Hz; 2H), 3.72 (s, 3H), 3.60-3.56 (m, 4H), 3.49-3.45 (m, 4H), 2.38 (d, J=6.4 Hz; 2H), 1.88-1.81 (m, 1H), 0.98 (d, J=6.8 Hz; 6H).

### Step 2

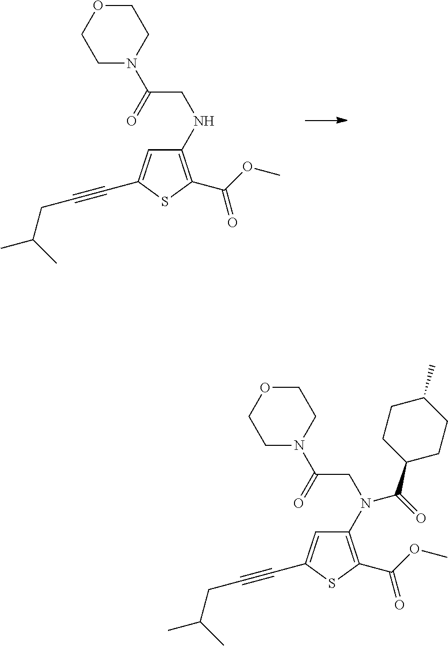

### Methyl 3-[(trans-4-methylcyclohexanecarbonyl)-(2-morpholino-2-oxo-ethyl)amino]-5-(4-methylpent-1-ynyl)thiophene-2-carboxylate

To a solution of methyl 5-(4-methylpent-1-ynyl)-3-[(2-morpholino-2-oxo-ethyl)amino]thiophene-2-carboxylate (120 mg, 0.329 mmol) and Et₃N (0.7 mL) in CH₂Cl₂ (5.0 mL) at 0° C. was added dropwise a solution of trans 4-methylcyclohexyl carbonyl chloride (530 mg, 3.27 mmol) in CH₂Cl₂ (5.0 mL), then the reaction stirred at 90° C. for 16 h. Reaction progress was monitored by TLC. Upon completion, the mixture was diluted with EtOAc (50 mL), washed with 2N aq. HCl (2×10 mL), water (10 mL), 10% NaHCO₃ solution (10 mL), brine (10 mL), dried over Na₂SO₄, and concentrated. The residue was purified (column chromatography, 100-200 mesh silica gel, 40% EtOAc in petroleum ether as eluent) to afford methyl 3-[(trans-4-methylcyclohexanecarbonyl)-(2-morpholino-2-oxo-ethyl)amino]-5-(4-methylpent-1-ynyl)thiophene-2-carboxylate (60 mg, 37%, yellow solid). TLC: 40% EtOAc in petroleum ether, R_{f}: 0.35. Analysis by LCMS (column: HALOC-18 (2.1×50 mm, 2.7 μm), mobile phase: A: 5 mM aq. CH₃CO₂NH₄, B: MeCN, time (min)/% B gradient: 0 min/3%, 0.5/3, 2/90, 2.5/90, 3.0/100, 3.5/50, flow rate: 0.5 mL/min, sample diluent: MeOH): MH⁺=489.5. t_{R}=2.84 min. ¹H NMR (400 MHz, DMSO-d6): 7.24 (s, 1H), 4.92 (d, J=16.8 Hz; 1H), 3.86 (d, J=16.8 Hz; 1H), 3.77 (s, 3H), 3.54 (br s, 4H), 3.44-3.38 (m, 4H), 2.41 (d, J=6.4 Hz; 2H), 2.12-2.02 (m, 1H), 1.89-1.85 (m, 1H), 1.67-1.55 (m, 4H), 1.29-1.23 (m, 4H), 0.98 (d, J=6.8 Hz; 6H),

### Step 3

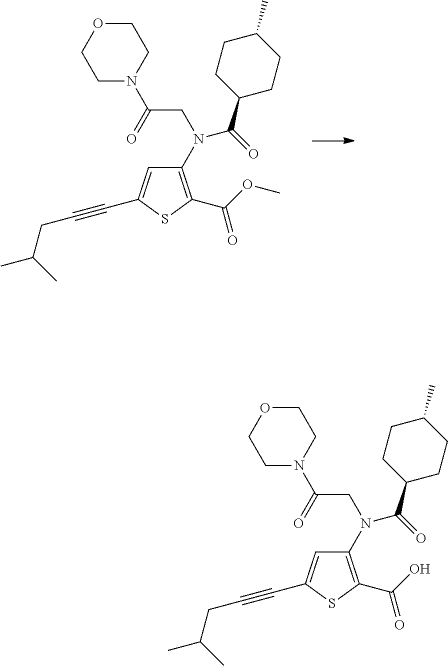 [SMILES: COC(=O)c1sc(C#CCC(C)C)cc1N(CC(=O)N1CCOCC1)C(=O)[C@H]1CC[C@H](C)CC1 ; CC(C)CC#Cc1cc(N(CC(=O)N2CCOCC2)C(=O)[C@H]2CC[C@H](C)CC2)c(C(=O)O)s1]

### 3-[(trans-4-methylcyclohexanecarbonyl)-(2-morpholino-2-oxo-ethyl)amino]-5-(4-methylpent-1-ynyl)thiophene-2-carboxylic acid

To a stirred RT solution of methyl 3-[(trans-4-methylcyclohexanecarbonyl)-(2-morpholino-2-oxo-ethyl)amino]-5-(4-methylpent-1-ynyl)thiophene-2-carboxylate (60 mg, 0.12 mmol) in 1:1 THF/water (4 mL) was added LiOH.H₂O (16 mg, 0.36 mmol) and the mixture stirred for 16 h. Reaction progress was monitored by TLC. Upon completion, the reaction was acidified (pH˜1) with 1M aq. HCl, extracted with EtOAc (75 mL). The organic layer was washed with water (2×10 mL), brine (10 mL), dried over Na₂SO₄, and concentrated to afford 130, 3-[(trans-4-methylcyclohexanecarbonyl)-(2-morpholino-2-oxo-ethyl)amino]-5-(4-methylpent-1-ynyl)thiophene-2-carboxylic acid (35 mg, 60%, yellow solid). TLC: 10% MeOH in CHCl₃, R_{f}: 0.33. Analysis by LCMS (column: X-Bridge Shield RP C-18 (4.6×75 mm, 3.5 μm), mobile phase: A: 0.01% aq. HCOOH, B: MeCN, time (min)/% B gradient: 0 min/5%, 3/95, 6.5/95, 7/100, 8/5, flow rate: 0.8 mL/min, sample diluent: MeCN): MH⁺=475.2, t_{R}=5.44 min. ¹H NMR (400 MHz, DMSO-d6): 13.5 (br s, exchanged with D₂O; 1H), 7.22 (s, 1H), 4.91 (d, J=16.8 Hz; 1H), 3.86 (d, J=16.8 Hz; 1H), 3.34-3.40 (m, 8H), 2.41 (d, J=6.4 Hz; 2H), 2.11-2.06 (m, 1H), 1.90-1.83 (m, 1H), 1.65-1.52 (m, 3H), 1.38-1.23 (m, 3H), 0.99 (s, 3H),

### Preparation of Compound 131

### Step 1

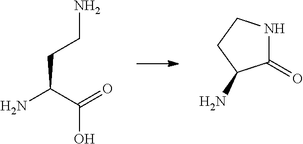 [SMILES: NCC[C@H](N)C(=O)O ; N[C@H]1CCNC1=O]

### (3S)-3-aminopyrrolidin-2-one

A suspension of (S)-2,4-diaminobutyric acid dihydrochloride (5.0 g, 27 mmol) and HMDS (65 mL) in MeCN (100 mL) was stirred at reflux for 48 h. Reaction progress was monitored by TLC. After completion the reaction mixture was cooled in ice, quenched with MeOH (25 mL), concentrated, and the residue extracted with hot CHCl₃ (3×150 mL). The combined CHCl₃ layer was concentrated to afford (3S)-3-aminopyrrolidin-2-one (2.2 g, 82%, brown solid). TLC: 30% MeOH—CHCl₃, R_{f}=0.5. MH⁺=100.9. ¹H NMR (DMSO-d6, 400 MHz): 7.62 (bs, exchanged with D₂O; 1H), 3.21 (t, J=8.3 Hz; 1H), 3.15-3.04 (m, 2H), 2.26-2.07 (m, 1H), 1.92 (bs, exchanged with D₂O; 1H), 1.70-1.57 (m, 1H).

### Step 2

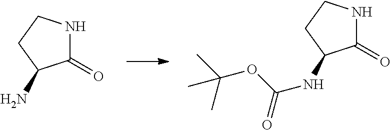

### Tert-butyl N-[(3S)-2-oxopyrrolidin-3-yl]carbamate

To a solution of (3S)-3-aminopyrrolidin-2-one (2.0 g, 20 mmol) and Et₃N (3.0 mL, 22 mmol) in CH₂Cl₂ (25 mL), boc-anhydride (4.36 g, 20.0 mmol) was added and the reaction stirred at RT for 16 h. Reaction progress was monitored by TLC. After completion the mixture was diluted with CH₂Cl₂ (50 mL), washed with water (2×20 mL), brine (20 mL), dried over Na₂SO₄, and concentrated. The residue was triturated with ether (2×20 mL), and the resulting solid dissolved in CH₂Cl₂ (50 mL); filtration and concentration afford tert-butyl N-[(3S)-2-oxopyrrolidin-3-yl]carbamate (2.8 g, 70%, brown solid). TLC: 10% MeOH—CHCl₃, R_{f}=0.35. ¹H NMR (CDCl₃, 400 MHz): 6.16 (bs, 1H), 5.10 (bs, 1H), 4.15 (m, 1H), 3.40-3.30 (m, 2H), 2.69-2.68 (m, 1H), 2.00-1.94 (m, 1H), 1.45 (s, 9H).

### Step 3

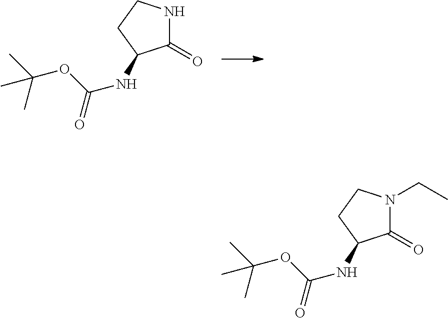 [SMILES: CC(C)(C)OC(=O)N[C@H]1CCNC1=O ; CCN1CC[C@H](NC(=O)OC(C)(C)C)C1=O]

### Tert-butyl N-[(3S)-1-ethyl-2-oxo-pyrrolidin-3-yl]carbamate

To a stirred RT suspension of sodium hydride (300 mg, 7.5 mmol) in THF (20 mL), a solution of tert-butyl N-[(3S)-2-oxopyrrolidin-3-yl]carbamate (1.0 g, 5.0 mmol) in DMF (6 mL) was added over 10 min, followed by ethyl iodide (0.406 mL, 5.0 mmol) in THF (10 mL). The reaction was then stirred at RT for 16 h. Reaction progress was monitored by TLC. Upon completion, the mixture was diluted with EtOAc (200 mL), washed with water (2×50 mL), brine (30 mL), dried over Na₂SO₄, and concentrated. The residue was purified (column chromatography, silica gel, 100-200 mesh, 2% MeOH in CHCl₃) to afford tert-butyl N-[(3S)-1-ethyl-2-oxo-pyrrolidin-3-yl]carbamate (550 mg, 48%, pale brown liquid). TLC: 10% MeOH in CHCl₃, R_{f}: 0.57. Analysis by LCMS (column: Sunfire C-18 (4.6×250 mm, 5 μm), mobile phase: A: 0.01 M aq. CH₃CO₂NH₄, B: MeCN, time (min)/% B gradient: 0 min/30%, 8/80, 15/80, 15.1/30, flow rate: 1.0 mL/min, sample diluent: MeCN): MH⁺=229.8. t_{R}=5.78 min. ¹H NMR (400 MHz, CDCl₃): 5.13 (br s, 1H), 4.14 (br s, 1H), 3.43-3.28 (m, 4H), 2.64-2.63 (m, 1H), 1.86-1.76 (m, 3H), 1.45 (s, 9H), 1.13 (t, J=7.2 Hz; 3H).

### Step 4

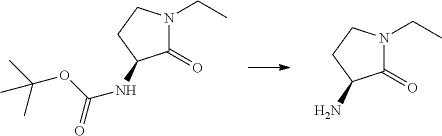

### (3S)-3-amino-1-ethyl-pyrrolidin-2-one

To a solution of tert-butyl N-[(3S)-1-ethyl-2-oxo-pyrrolidin-3-yl]carbamate (550 mg, 2.41 mmol) in dichloromethane (5 mL), 2M HCl in Et₂O (5 mL) was added at 0° C., then the mixture stirred at RT for 16 h. Reaction progress was monitored by TLC. Upon completion, the solution was concentrated to afford (3S)-3-amino-1-ethyl-pyrrolidin-2-one (400 mg, 71%, brown oil). Analysis by LCMS (column: Sunfire C-18 (4.6×250 mm, 5 μm), mobile phase: A: 0.05% aq. TFA, B: MeCN, time (min)/% B gradient: 0 min/20%, 10/80, 15/80, 15.1/20, flow rate: 1.0 mL/min, sample diluent: 1:1 A & B): MH⁺=129.2. t_{R}=2.56 min. [α]_{D}: −27.8 deg. (c=1.15% in MeOH). ¹H NMR (400 MHz, DMSO-d6): 8.64 (br s, exchanged with D₂O; 1H), 3.95-3.89 (m, 1H), 3.41-3.20 (m, 4H), 2.41-2.34 (m, 1H), 2.01-1.93 (m, 1H), 1.05 (t, J=7.2 Hz; 3H).

### Step 5

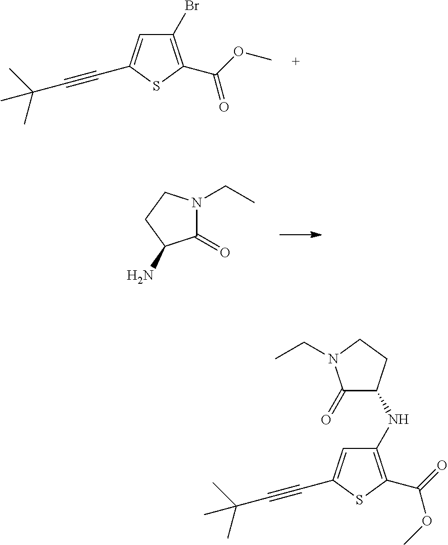

### Methyl 5-(3,3-dimethylbut-1-ynyl)-3-[[(3S)-1-ethyl-2-oxo-pyrrolidin-3-yl]amino]thiophene-2-carboxylate

A suspension of methyl-3-bromo-5-(3,3-dimethylbut-1-ynyl)thiophene-2-carboxylate (500 mg, 1.66 mmol), (3S)-3-amino-1-ethyl-pyrrolidin-2-one (500 mg, 1.66 mmol), and Cs₂CO₃ (1.62 g, 4.98 mmol) in toluene (10 mL) was deoxygenated by purging with a stream of argon for 60 min, after which Pd(OAc)₂ (37 mg, 0.17 mmol) and (±)BINAP (103 mg, 0.166 mmol) were added and purging was continued for another 30 min. The reaction was then stirred at 100° C. for 16 h. Reaction progress was monitored by TLC. After completion the mixture was cooled to RT, diluted with EtOAc (100 mL), and filtered through celite. The filtrate was washed with water (2×20 mL), brine (20 mL), dried over Na₂SO₄, and concentrated. The resulting residue was purified (column chromatography, silica gel, 100-200 mesh, 60% EtOAc in petroleum ether) to afford methyl 5-(3,3-dimethylbut-1-ynyl)-3-[[(3S)-1-ethyl-2-oxo-pyrrolidin-3-yl]amino]thiophene-2-carboxylate (300 mg, 51%, pale brown solid). TLC: 50% EtOAc in petroleum ether, R_{f}: 0.44. Analysis by LCMS (column: Xterra RP-18 (4.6×100 mm, 5 μm), mobile phase: A: 0.01 M aq. CH₃CO₂NH₄, B: MeCN, time (min)/% B gradient: 0 min/10%, 4/70, 6/95, 14/95, 15/10, flow rate: 0.8 mL/min, sample diluent: MeCN): MH⁺=349.1. t_{R}=7.19 min. [α]_{D}: 14.9 deg. (c=1.01% in MeOH), chiral purity 93.0% (288 nm, Chiralpak AD-H column (4.6×250 mm, 5 μm), mobile phase: 30% EtOH (0.1% TFA)/70% hexane (0.1% TFA), 1.0 mL/min, RT). ¹H NMR (400 MHz, DMSO-d6): 6.95 (s, 1H), 6.93 (d, J=6.8 Hz; exchanged with D₂O; 1H), 4.33-4.27 (m, 1H), 3.72 (s, 3H), 3.36-3.19 (m, 4H), 2.60-2.50 (m, 1H), 1.83-1.73 (m, 1H), 1.43 (s, 9H), 1.05 (t, J=7.2 Hz; 3H).

### Step 6

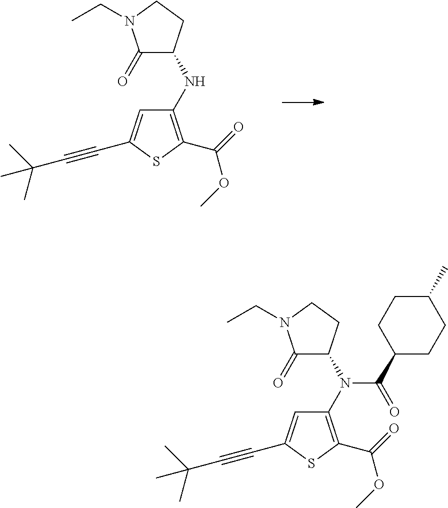 [SMILES: CCN1CC[C@H](Nc2cc(C#CC(C)(C)C)sc2C(=O)OC)C1=O ; CCN1CC[C@H](N(c2cc(C#CC(C)(C)C)sc2C(=O)OC)C(=O)[C@H]2CC[C@H](C)CC2)C1=O]

### Methyl 5-(3,3-dimethylbut-1-ynyl)-3-[[(3S)-1-ethyl-2-oxo-pyrrolidin-3-yl]-(trans-4-methylcyclohexanecarbonyl)amino]thiophene-2-carboxylate

To a stirred solution of methyl 5-(3,3-dimethylbut-1-ynyl)-3-[[(3S)-1-ethyl-2-oxo-pyrrolidin-3-yl]amino]thiophene-2-carboxylate (220 mg, 0.632 mmol), pyridine (2.0 mL, 10 vol), and DMAP (38 mg, 0.32 mmol) in dichloroethane (5 mL) at 0° C., was added dropwise a stock solution of 4-Methylcylohexanecarbonyl chloride (0.898 g, 6.32 mmol) in dichloroethane (5 mL). After addition the reaction mixture was stirred at 100° C. for 16 h. Reaction progress was monitored by TLC. Upon completion, the reaction mixture was diluted with EtOAc (50 mL), washed with 2N HCl (10 mL), 10% NaHCO₃ solution (3×20 mL), water (10 mL), brine (20 mL), dried over Na₂SO₄, and concentrated. The resulting residue was purified (column chromatography, 100-200 mesh silica gel, 40% EtOAc in petroleum ether) to afford methyl 5-(3,3-dimethylbut-1-ynyl)-3-[[(3S)-1-ethyl-2-oxo-pyrrolidin-3-yl]-(trans-4-methylcyclohexanecarbonyl)amino]thiophene-2-carboxylate (200 mg, 67%, off-white solid). TLC: 40% EtOAc in petroleum ether, R_{f}: 0.3. Analysis by LCMS (column: HALO C-18 (2.1×100 mm, 2.7 μm), mobile phase: A: 5 mM aq. CH₃CO₂NH₄, B: MeCN, time (min)/% B gradient: 0 min/3%, 0.5/3, 2/90, 2.5/90, 3.0/100, 3.5/50, flow rate: 0.5 mL/min, sample diluent: MeOH): MH⁺=473.5. t_{R}=2.89 min. [α]_{D}: −15.4 deg. (c=1.0% in MeOH), chiral purity 97.6% (300 nm, Chiralpak AD-H column (4.6×250 mm, 5 μm), mobile phase: 15% EtOH/85% hexane, 0.8 mL/min, RT). ¹H NMR (400 MHz, DMSO-d6): 7.18 (s, 0.39H), 7.01 (s, 0.55H), 5.47 (t, J=9.2 Hz; 0.6H), 4.17-4.13 (m, 0.44H), 3.76 (s, 3H), 3.25-3.05 (m, 3H), 3.03-2.67 (m, 1H), 2.32-2.19 (m, 1H), 2.11-1.95 (m, 2H), 1.59 (m, 6H), 1.28-1.27 (m, 10H), 1.23-0.99 (m, 2H), 0.97-0.92 (m).

### Step 7

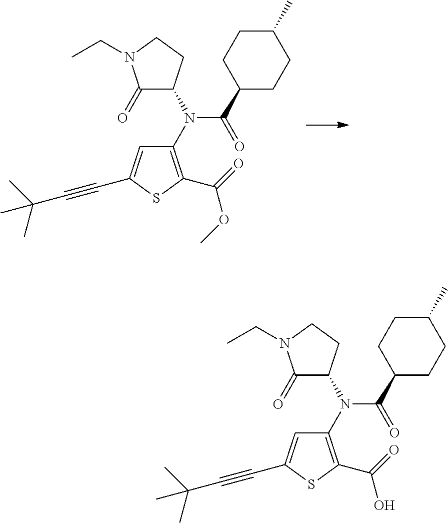

### 5-(3,3-dimethylbut-1-ynyl)-3-[[(3S)-1-ethyl-2-oxo-pyrrolidin-3-yl]-(trans-4-methylcyclohexanecarbonyl)amino]thiophene-2-carboxylic acid

To a stirred RT solution of methyl 5-(3,3-dimethylbut-1-ynyl)-3-[[(3S)-1-ethyl-2-oxo-pyrrolidin-3-yl]-(trans-4-methylcyclohexanecarbonyl)amino]thiophene-2-carboxylate (200 mg, 0.423 mmol) in a 1:1 THF/water (6 mL), was added LiOH.H₂O (53.3 mg, 1.59 mmol) and the reaction stirred for 3 h. Reaction progress was monitored by TLC. Upon completion, the pH was then adjusted to ˜1 using 1M aq. HCl, then the mixture extracted with EtOAc (50 mL), washed with water (3×20 mL), brine (20 mL), dried over Na₂SO₄, and concentrated to afford 131, 5-(3,3-dimethylbut-1-ynyl)-3-[[(3S)-1-ethyl-2-oxo-pyrrolidin-3-yl]-(trans-4-methylcyclohexanecarbonyl)amino]thiophene-2-carboxylic acid (100 mg, 51%, off-white solid). TLC: 10% MeOH in CHCl₃, R_{f}: 0.4. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 3.0/50, 3.0/100, 3.5/100, 4.0/100, flow rate: 0.5 mL/min, sample diluent: MeOH): MH⁺=457.5. t_{R}=1.41 min. [α]_{D}: −9.2 deg. (c=1.0% in MeOH), chiral purity 87.7% (300 nm, Chiralpak IC column (4.6×250 mm), mobile phase: 20% EtOH (0.1% DEA)/80% hexane (0.1% DEA), 0.8 mL/min, 40° C.). ¹H NMR (400 MHz, DMSO-d6): 13.60 (br s, exchanged with D₂O; 1H), 7.12 (s, 0.33H), 6.97 (s, 0.65H), 5.50 (t, J=9.6 Hz; 0.64H), 4.10-4.02 (m, 0.47H), 3.32-3.11 (m, 3H), 3.04-3.00 (s, 1H), 2.41-1.90 (m, 3H), 1.60-1.45 (m, 5H), 1.35-1.01 (m, 11H), 1.01-0.86 (m, 3H).

### Preparation of Compound 132

### Step 1

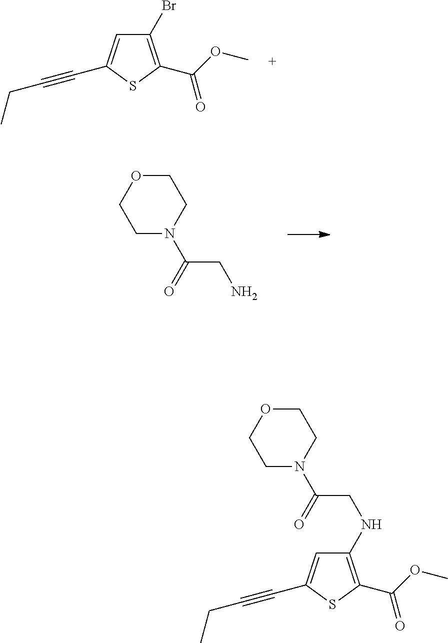 [SMILES: CCC#Cc1cc(Br)c(C(=O)OC)s1 ; NCC(=O)N1CCOCC1 ; CCC#Cc1cc(NCC(=O)N2CCOCC2)c(C(=O)OC)s1]

### Methyl 5-but-1-ynyl-3-[(2-morpholino-2-oxo-ethyl)amino]thiophene-2-carboxylate

A suspension of methyl 3-bromo-5-(but-1-ynyl)thiophene-2-carboxylate (prepared as precedented, 2.0 g, 7.3 mmol), 2-amino-1-morpholinoethanone.HCl (1.98 g, 11.0 mmol) and Cs₂CO₃ (7.16 g, 22.0 mmol) in toluene (30 mL) was deoxygenated by bubbling with argon for 50 min, then Pd(OAc)₂ (164 mg, 0.732 mmol) and (±)-BINAP (455 mg, 0.732 mmol) were added. The purging was continued for an additional 10 min and the mixture stirred at 100° C. for 16 h. Reaction progress was monitored by TLC. Upon completion, the reaction was cooled to RT, diluted with EtOAc (100 mL), and filtered through celite. The filtrate was washed with water (2×50 mL), brine (30 mL), dried over Na₂SO₄, and concentrated. The residue was purified (silica gel column chromatography, 100-200 mesh, 60% EtOAc in petroleum ether) to afford methyl 5-but-1-ynyl-3-[(2-morpholino-2-oxo-ethyl)amino]thiophene-2-carboxylate (1.0 g, 40%, yellow solid). TLC: 60% EtOAc in petroleum ether, R_{f}: 0.35. MH⁺=337.1. ¹H NMR (400 MHz, DMSO-d6): 7.24 (s, exchanged with D₂O; 1H), 6.99 (s, 1H), 4.14 (d, J=4.8 Hz; 2H), 3.72 (s, 3H), 3.60-3.56 (m, 4H), 3.48-3.44 (m, 4H), 2.50-2.44 (m, 2H), 1.15 (t, J=7.6 Hz; 3H).

### Step 2

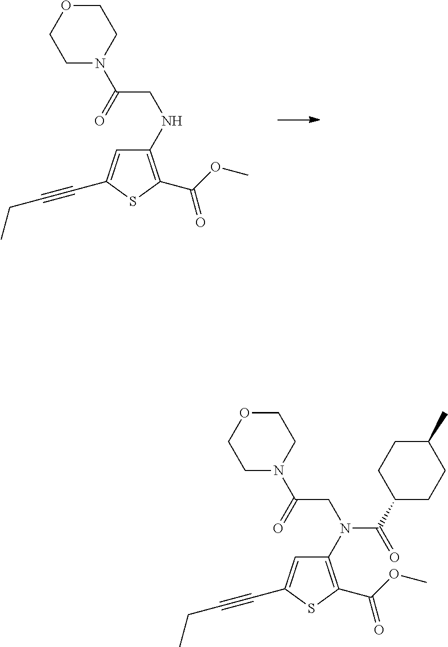 [SMILES: CCC#Cc1cc(NCC(=O)N2CCOCC2)c(C(=O)OC)s1 ; CCC#Cc1cc(N(CC(=O)N2CCOCC2)C(=O)[C@H]2CC[C@H](C)CC2)c(C(=O)OC)s1]

### Methyl 5-but-1-ynyl-3-[(trans-4-methylcyclohexanecarbonyl)-(2-morpholino-2-oxo-ethyl)amino]thiophene-2-carboxylate

To a 0° C. solution of methyl 5-but-1-ynyl-3-[(2-morpholino-2-oxo-ethyl)amino]thiophene-2-carboxylate (300 mg, 0.892 mmol), pyridine (3.0 mL), and DMAP (55 mg, 0.44 mmol) in DCE (5 mL) was added dropwise a solution of trans 4-methylcyclohexyl carbonyl chloride (1.26 g, 8.92 mmol) in DCE (10 mL), then the reaction stirred at 90° C. for 16 h. Reaction progress was monitored by TLC. Upon completion, the mixture was diluted with EtOAc (80 mL), washed with 2N aq. HCl (30 mL), 10% NaHCO₃ solution (2×20 mL), water (10 mL), brine (20 mL), dried over Na₂SO₄, and concentrated. The residue was purified (silica gel column chromatography, 100-200 mesh, 50% EtOAc in petroleum ether as eluent) to afford methyl 5-but-1-ynyl-3-[(trans-4-methylcyclohexanecarbonyl)-(2-morpholino-2-oxo-ethyl)amino]thiophene-2-carboxylate (350 mg, 85%, yellow solid). TLC: 60% EtOAc in petroleum ether, R_{f}: 0.3. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, 3/3 flow rate: 0.4 mL/min, sample diluent: MeOH): MH⁺=461.2. t_{R}=1.91 min. ¹H NMR (400 MHz, DMSO-d6): 7.27 (s, 1H), 4.92 (d, J=16.4 Hz; 1H), 3.85 (d, J=16.8 Hz; 1H), 3.77 (s, 3H), 3.54-3.58 (m, 4H), 3.44-3.38 (m, 4H), 2.53-2.47 (m, 2H), 2.10-2.04 (m, 1H), 1.67-1.55 (m, 3H), 1.50-1.47 (m, 1H), 1.41-1.21 (m, 3H), 1.16 (t, J=7.6 Hz; 3H), 0.95.

### Step 3

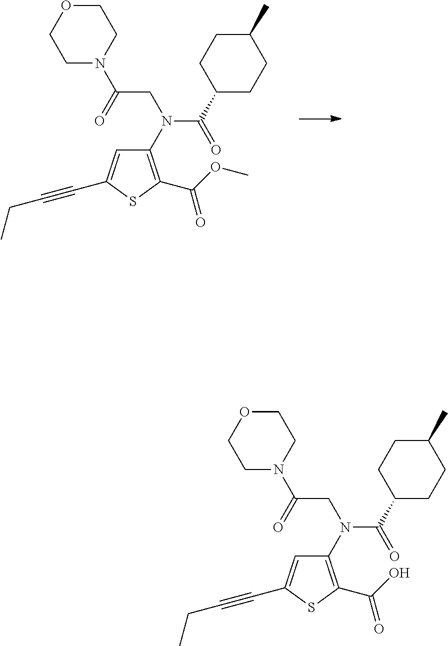 [SMILES: CCC#Cc1cc(N(CC(=O)N2CCOCC2)C(=O)[C@H]2CC[C@H](C)CC2)c(C(=O)OC)s1 ; CCC#Cc1cc(N(CC(=O)N2CCOCC2)C(=O)[C@H]2CC[C@H](C)CC2)c(C(=O)O)s1]

### 5-But-1-ynyl-3-[(trans-4-methylcyclohexanecarbonyl)-(2-morpholino-2-oxo-ethyl)amino]thiophene-2-carboxylic acid

To a stirred solution of methyl 5-but-1-ynyl-3-[(trans-4-methylcyclohexanecarbonyl)-(2-morpholino-2-oxo-ethyl)amino]thiophene-2-carboxylate (350 mg, 0.76 mmol) in 1:1 THF/water (6 mL), LiOH.H₂O (95 mg, 2.3 mmol) was added at RT and the reaction stirred for 16 h. Reaction progress was monitored by TLC. Upon completion, the mixture was acidified (pH˜1) with 1M aq. HCl, extracted with EtOAc (50 mL), and the resulting organic layer washed with water (20 mL), brine (20 mL), dried over Na₂SO₄, and concentrated to afford 132,5-But-1-ynyl-3-[(trans-4-methylcyclohexanecarbonyl)-(2-morpholino-2-oxo-ethyl)amino]thiophene-2-carboxylic acid (145 mg, 42%, yellow solid). TLC: 10% MeOH in CHCl₃, R_{f}: 0.5. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, 3/3, flow rate: 0.4 mL/min, sample diluent: MeOH): MH⁺=445.1. t_{R}=1.24 min. ¹H NMR (400 MHz, DMSO-d6): 13.5 (s, exchanged with D₂O; 1H), 7.24 (s, 1H), 4.92 (d, J=16.4 Hz; 1H), 3.85 (d, J=16.4 Hz; 1H), 3.54-3.53 (m, 4H), 3.39-3.31 (m, 4H), 2.52-2.46 (m, 2H), 2.08-2.05 (m, 1H), 1.65-1.51 (m, 4H), 1.38-1.23 (m, 3H), 1.16 (t, J=3.6 Hz; 3H), 0.7.

### Preparation of Compound 133

### Steps 1-2

### As in Compound 131

### Step 3

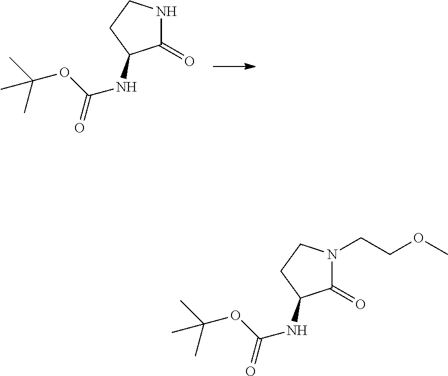 [SMILES: CC(C)(C)OC(=O)N[C@H]1CCNC1=O ; COCCN1CC[C@H](NC(=O)OC(C)(C)C)C1=O]

### Tert-butyl N-[(3S)-1-(2-methoxyethyl)-2-oxo-pyrrolidin-3-yl]carbamate

To a suspension of sodium hydride (600 mg, 15 mmol) in THF (30 mL), a solution of tert-butyl N-[(3S)-2-oxopyrrolidin-3-yl]carbamate (2.0 g, 10 mmol) in DMF (40 mL) was added at 0° C. over 10 min. A solution of (2-bromoethyl)methyl ether (0.94 mL, 10 mmol) in THF (10 mL) was then added and the reaction stirred at RT for 16. Reaction progress was monitored by TLC. Upon completion, the reaction mixture was diluted with EtOAc (200 mL), washed with water (2×50 mL), brine (30 mL), dried over Na₂SO₄, and concentrated. The residue was purified (silica gel column chromatography, 100-200 mesh, MeOH in CHCl₃ as eluent) to afford tert-butyl N-[(3S)-1-(2-methoxyethyl)-2-oxo-pyrrolidin-3-yl]carbamate (800 mg, 31%, yellow gum). TLC: 10% MeOH in CHCl₃, R_{f}: 0.42. MH⁺=259.2. ¹H NMR (400 MHz, DMSO-d6): 7.07 (d, J=8.8 Hz; 1H), 4.10-4.03 (m, 1H), 3.41 (s, 3H), 3.29-3.19 (m, 6H), 2.24-2.17 (m, 1H), 1.80-1.72 (m, 1H), 1.38 (s, 9H).

### Step 4

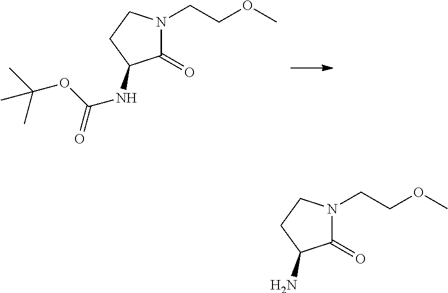 [SMILES: COCCN1CC[C@H](NC(=O)OC(C)(C)C)C1=O ; COCCN1CC[C@H](N)C1=O]

### (3S)-3-amino-1-(2-methoxyethyl)pyrrolidin-2-one

To a solution of tert-butyl N-[(3S)-1-(2-methoxyethyl)-2-oxo-pyrrolidin-3-yl]carbamate (800 mg, 3.10 mmol) in dichloromethane (10 mL), 2M HCl in Et₂O (10 mL) was added at 0° C. and the reaction stirred at RT for 16 h. Reaction progress was monitored by TLC. Upon completion, the reaction mixture was concentrated to afford (3S)-3-amino-1-(2-methoxyethyl)pyrrolidin-2-one (600 mg, 99%, brown gum). MH⁺=159.2. ¹H NMR (400 MHz, DMSO-d6): 8.49 (br s, 2H), 3.96-3.95 (m, 1H), 3.45 (s, 3H), 3.44-3.31 (m, 2H), 3.30 (br s, 3H), 2.40-2.33 (m, 1H), 1.94-1.89 (m, 1H).

### Step 5

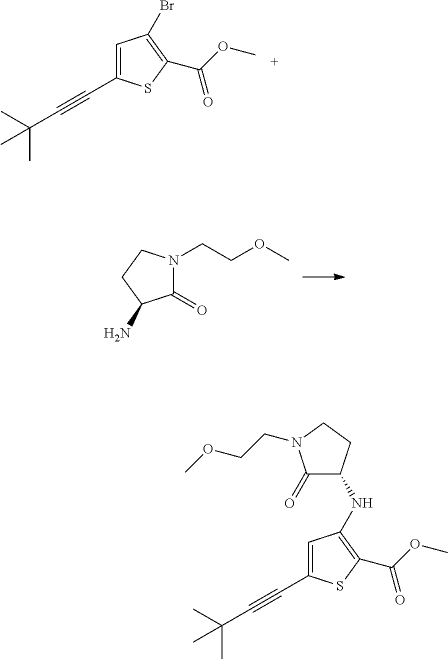

### Methyl 5-(3,3-dimethylbut-1-ynyl)-3-[[(3S)-1-(2-methoxyethyl)-2-oxo-pyrrolidin-3-yl]amino]thiophene-2-carboxylate

A suspension of methyl-3-bromo-5-(3, 3-dimethylbut-1-ynyl)thiophene-2-carboxylate (773 mg, 2.57 mmol), (3S)-3-amino-1-(2-methoxyethyl)pyrrolidin-2-one (750 mg, 3.86 mmol), and Cs₂CO₃ (2.51 g, 7.71 mmol) in toluene (15 mL) was deoxygenated by purging a stream of argon for 60 min, after which Pd(OAc)₂ (57.7 mg, 0.257 mmol) and (±)-BINAP (160 mg, 0.257 mmol) were added. Purging was continued for another 30 min, then the reaction was stirred at 100° C. for 16 h. Reaction progress was monitored by TLC. Upon completion, the reaction was cooled to RT, diluted with EtOAc (100 mL), and filtered through celite. The resulting filtrate was washed with water (2×50 mL), brine (20 mL), dried over Na₂SO₄, and concentrated. The residue was purified (silica gel column chromatography, 100-200 mesh, 60% EtOAc in petroleum ether) to afford methyl 5-(3,3-dimethylbut-1-ynyl)-3-[[(3S)-1-(2-methoxyethyl)-2-oxo-pyrrolidin-3-yl]amino]thiophene-2-carboxylate (650 mg, 67%, orange gum). TLC: 50% EtOAc in petroleum ether, R_{f}: 0.25. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=379.2. t_{R}=1.89 min. Chiral purity: 99.5% at max plot (Chiralpak AD-H column (4.6×250 mm, 5 μm), mobile phase: 20% EtOH/80% hexane, 0.8 mL/min, RT). ¹H NMR (400 MHz, DMSO-d6): 6.96-6.93 (m, 1H exchanged with D₂O; 2H), 4.35-4.28 (m, 1H), 3.72 (s, 3H), 3.47-3.44 (m, 2H), 3.41-3.31 (m, 4H), 3.26 (s, 3H), 2.58-2.56 (m, 1H), 1.81-1.76 (m, 1H), 1.27 (s, 9H).

### Step 6

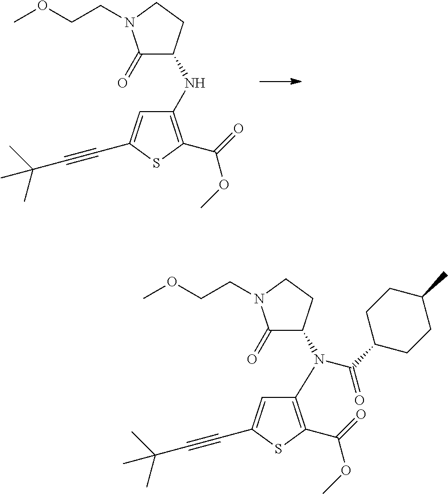 [SMILES: COCCN1CC[C@H](Nc2cc(C#CC(C)(C)C)sc2C(=O)OC)C1=O ; COCCN1CC[C@H](N(c2cc(C#CC(C)(C)C)sc2C(=O)OC)C(=O)[C@H]2CC[C@H](C)CC2)C1=O]

### Methyl 5-(3,3-dimethylbut-1-ynyl)-3-[[(3S)-1-(2-methoxyethyl)-2-oxo-pyrrolidin-3-yl]-(trans-4-methylcyclohexanecarbonyl)amino]thiophene-2-carboxylate

To a 0° C. stirred solution of methyl 5-(3,3-dimethylbut-1-ynyl)-3-[[(3S)-1-(2-methoxyethyl)-2-oxo-pyrrolidin-3-yl]amino]thiophene-2-carboxylate 3 (650 mg, 1.72 mmol), pyridine (6.5 mL, vol), and DMAP (105 mg, 0.858 mmol) in dichloroethane (30 mL) was added dropwise a solution of 4-methylcylohexanecarbonyl chloride (2.75 g, 17.2 mmol) in dichloroethane (10 mL) and the mixture stirred at 100° C. for 16 h. Reaction progress was monitored by TLC. Upon completion the reaction mixture was diluted with EtOAc (150 mL), washed with 2N HCl (40 mL), 10% NaHCO₃ solution (3×50 mL), water (30 mL), brine (20 mL), dried over Na₂SO₄, and concentrated. The resulting residue was purified (silica gel column chromatography, 100-200 mesh, 50% EtOAc in petroleum ether) to afford methyl 5-(3,3-dimethylbut-1-ynyl)-3-[[(3S)-1-(2-methoxyethyl)-2-oxo-pyrrolidin-3-yl]-(trans-4-methylcyclohexanecarbonyl)amino]thiophene-2-carboxylate (350 mg, 67%, pink solid). TLC: 60% EtOAc in petroleum ether, R_{f}: 0.28. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=503.5. t_{R}=2.18 min. Chiral purity: 99.7% (300 nm, Chiralpak AD-H column (4.6×250 mm, 5 μm), mobile phase: 20% EtOH (0.1% DEA)/80% hexane 0.1% DEA), 0.8 mL/min, 40° C.). ¹H NMR (400 MHz, DMSO-d6): 7.18 (s, 0.3H), 7.02 (s, 0.7H), 5.54-5.49 (m, 0.7H), 4.23-4.19 (m, 0.3H), 3.76 (s, 3H), 3.39-3.32 (m, 3H), 3.28-3.16 (m, 6H), 2.26-1.96 (m, 3H), 1.60-1.33 (m, 7H), 1.29 (s, 9H), 0.76 (d, J=6.4 Hz; 3H), 0.69-0.59 (m, 2H).

### Step 7

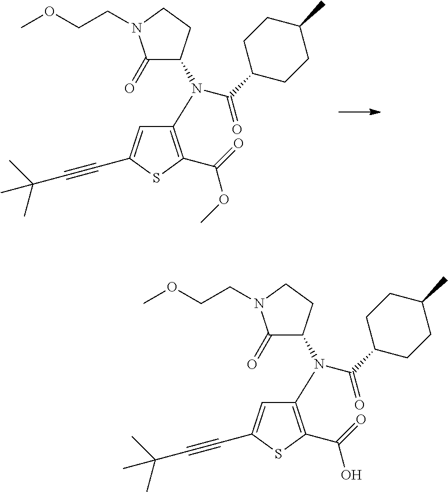

### 5-(3,3-Dimethylbut-1-ynyl)-3-[[(3S)-1-(2-methoxyethyl)-2-oxo-pyrrolidin-3-yl]-(trans-4-methylcyclohexanecarbonyl)amino]thiophene-2-carboxylic acid

To a stirred solution of methyl 5-(3,3-dimethylbut-1-ynyl)-3-[[(3S)-1-(2-methoxyethyl)-2-oxo-pyrrolidin-3-yl]-(trans-4-methylcyclohexanecarbonyl)amino]thiophene-2-carboxylate (300 mg, 0.597 mmol) in 1:1 THF/water (10 mL), LiOH.H₂O (53.3 mg, 0.895 mmol) was added at RT and the reaction stirred for 3 h. Reaction progress was monitored by TLC. Upon completion, the mixture was acidified to pH˜1 using 1M aq. HCl, extracted with EtOAc (50 mL), washed with water (3×20 mL), brine (20 mL), dried over Na₂SO₄, and concentrated to afford 133, 5-(3,3-Dimethylbut-1-ynyl)-3-[[(3S)-1-(2-methoxyethyl)-2-oxo-pyrrolidin-3-yl]-(trans-4-methylcyclohexanecarbonyl)amino]thiophene-2-carboxylic acid (120 mg, 41%, Off-white solid). TLC: 10% MeOH in CHCl₃, R_{f}: 0.3. Analysis by LCMS (column: HALO C-18 (2.1×100 mm, 2.7 μm), mobile phase: A: 5 mM aq. CH₃CO₂NH₄, B: MeCN, time (min)/% B gradient: 0 min/3%, 0.5/3, 2/90, 2.5/90, 3.0/100, 3.5/50, flow rate: 0.5 mL/min, sample diluent: MeOH): MH⁺=489.4. t_{R}=1.85 min. Chiral purity: 99.7% (292 nm, Chiralpak AD-H column (4.6×250 mm, 5 μm), mobile phase: 20% EtOH (0.1% DEA)/80% hexane 0.1% DEA), 0.8 mL/min, 40° C.). ¹H NMR (400 MHz, DMSO-d6): 13.5 (br s, exchanged with D₂O; 1H), 7.12 (s, 0.3H), 6.97 (s, 0.7H), 5.56-5.51 (m, 0.7H), 4.14-4.09 (m, 0.3H), 3.45-3.36 (m, 3H), 3.28-3.27 (m, 2H), 3.25-3.23 (m, 1H), 3.20-3.16 (m, 2H), 3.13-3.09 (m, 1H), 2.32-2.18 (m, 1H), 2.10-1.96.

### Preparation of Compound 139

### Steps 1-7

### As in Compound 133

### Step 8

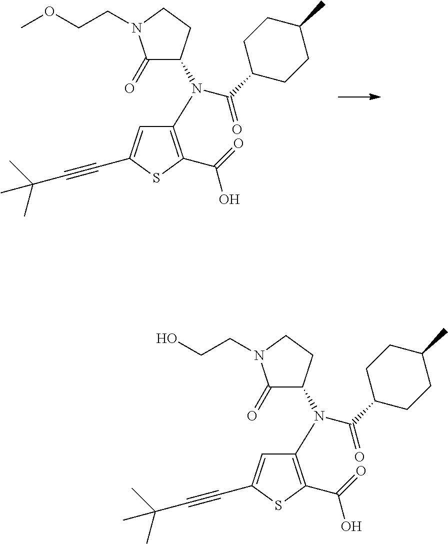

### 5-(3,3-Dimethylbut-1-ynyl)-3-[[(3S)-1-(2-hydroxyethyl)-2-oxo-pyrrolidin-3-yl]-(trans-4-methylcyclohexanecarbonyl)amino]thiophene-2-carboxylic acid

To a solution of 5-(3,3-Dimethylbut-1-ynyl)-3-[[(3S)-1-(2-methoxyethyl)-2-oxo-pyrrolidin-3-yl]-(trans-4-methylcyclohexanecarbonyl)amino]thiophene-2-carboxylic acid (80 mg, 0.16 mmol) in CH₂Cl₂ (2 mL), BBr₃ (0.050 mL, 0.49 mmol) was added at 0° C. and the mixture stirred at RT for 2 h. Reaction progress was monitored by TLC. Upon completion, the reaction was quenched with water (10 mL) and extracted with EtOAc (2×10 mL). The resulting organic layer was washed with water (10 mL), brine (10 mL), dried over Na₂SO₄, filtered, and concentrated. The residue was purified (prep. RP-HPLC with aq. HCOOH and acetonitrile as eluent) to afford 139, 5-(3,3-dimethylbut-1-ynyl)-3-[[(3S)-1-(2-hydroxyethyl)-2-oxo-pyrrolidin-3-yl]-(trans-4-methylcyclohexanecarbonyl)amino]thiophene-2-carboxylic acid (20 mg, 25%, pale green solid). TLC: 10% MeOH in CHCl₃, R_{f}: 0.2. Analysis by LCMS (column: BEH C-18 (2.1×50 mm, 1.7 μm), mobile phase: A: 0.1% aq. HCOOH, B: MeCN, time (min)/% B gradient: 0 min/3%, 0.1/3, 1.5/90, 1.8/90, 2.2/95, 3.2/95, 3.8/3, flow rate: 0.4 mL/min, sample diluent: MeOH): MH⁺=475.6. t_{R}=1.7 min. Chiral purity: 99.5% (292 nm, Chiralpak AD-H column (4.6×250 mm, 5 μm), mobile phase: 20% EtOH (0.1% DEA)/80% hexane 0.1% DEA), 0.8 mL/min, RT). ¹H NMR (400 MHz, DMSO-d6): 13.6 (br s, exchanged with D₂₀, 1H), 7.10 (s, 0.31H), 6.97 (s, 0.64H), 5.55 (t, J=10 Hz; 0.65H), 4.68-4.61 (m, exchanged with D₂₀, 1H), 4.05 (t, J=9.6 Hz; 0.30H), 3.45-3.35 (m, 4H), 3.13-3.03 (m, 4H), 2.19-1.95 (m, 3H), 1.57-1.33 (m, 6H).

### Preparation of Compound 141

### Steps 1-2

### As in Compound 131

### Step 3

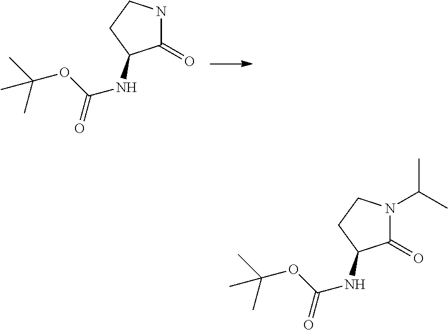 [SMILES: CC(C)(C)OC(=O)N[C@H]1CCNC1=O ; CC(C)N1CC[C@H](NC(=O)OC(C)(C)C)C1=O]

### Tert-butyl N-[(3S)-1-isopropyl-2-oxo-pyrrolidin-3-yl]carbamate

To a suspension of sodium hydride (300 mg, 7.5 mmol) in THF (20 mL), a solution of tert-butyl N-[(3S)-2-oxopyrrolidin-3-yl]carbamate (1.0 g, 5.0 mmol) in DMF (6 mL) was added at RT overl0 min, isopropyl iodide (0.75 mL, 7.5 mmol) in THF (10 mL) was then added and the reaction mixture stirred at RT for 6 h. Reaction progress was monitored by TLC. Upon completion, the mixture was diluted with EtOAc (200 mL), washed with water (2×50 mL), brine (30 mL), dried over Na₂SO₄, and concentrated. The residue was purified (silica gel column chromatography, 100-200 mesh, 2% MeOH in CHCl₃) to afford tert-butyl N-[(3S)-1-isopropyl-2-oxo-pyrrolidin-3-yl]carbamate. (200 mg, 16%, yellow liquid). TLC: 10% MeOH in CHCl₃, R_{f}: 0.74. ¹H-NMR (400 MHz, DMSO-d6): 7.04 (d, J=8.8 Hz; exchanged with D₂O; 1H), 4.11-1.05 (m, 2H), 3.25-3.20 (m, 1H), 3.20-3.11 (m, 1H), 2.23-2.18 (m, 1H), 1.38 (s, 9H), 1.07-1.03 (m, 6H).

### Step 4

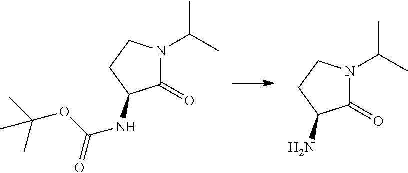

### (3S)-3-amino-1-isopropyl-pyrrolidin-2-one

To a solution of tert-butyl N-[(3S)-1-isopropyl-2-oxo-pyrrolidin-3-yl]carbamate. (490 mg, 2.15 mmol) in dichloromethane (10 mL), 2M HCl in Et₂O (6 mL) was added at 0° C. and the mixture stirred at RT for 16 h. Reaction progress was monitored by TLC. Upon completion, the solution was concentrated to afford (3S)-3-amino-1-isopropyl-pyrrolidin-2-one (350 mg, 96%, pale yellow liquid). ¹H NMR (400 MHz, DMSO-d6): 8.56 (br s, 1H), 4.16-4.10 (m, 1H), 3.95-3.89 (m, 1H), 3.40-3.36 (m, 1H), 3.27-3.23 (m, 1H), 2.39-2.34 (m, 1H), 1.92-1.88 (m, 1H), 1.12-1.07 (m, 6H).

### Step 5

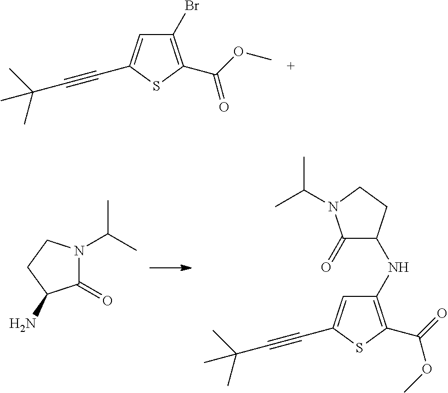

### Methyl 5-(3,3-dimethylbut-1-ynyl)-3-[(1-isopropyl-2-oxo-pyrrolidin-3-yl)amino]thiophene-2-carboxylate

A suspension of methyl-3-bromo-5-(3,3-dimethylbut-1-ynyl)thiophene-2-carboxylate (500 mg, 1.66 mmol), (3S)-3-amino-1-isopropyl-pyrrolidin-2-one (353 mg, 2.49 mmol), and Cs₂CO₃ (1.62 g, 4.98 mmol) in toluene (10 mL) was deoxygenated by purging a stream of argon for 60 min. Pd(OAc)₂ (37 mg, 0.17 mmol) and (±)BINAP (103 mg, 0.166 mmol) were then added, purging continued for another 30 min, then the reaction was stirred at 100° C. for 16 h. Reaction progress was monitored by TLC. After completion the reaction mixture was cooled to RT, diluted with EtOAc (100 mL), and filtered through celite. The filtrate was washed with water (2×20 mL), brine (20 mL), dried over Na₂SO₄, concentrated, and the resulting residue purified by silica gel column chromatography (100-200 mesh, 50% EtOAc in petroleum ether as eluent) to afford methyl 5-(3,3-dimethylbut-1-ynyl)-3-[(1-isopropyl-2-oxo-pyrrolidin-3-yl)amino]thiophene-2-carboxylate (260 mg, 41%, white solid). TLC: 50% EtOAc in petroleum ether, R_{f}: 0.42. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=363.4. t_{R}=2.01 min. Chiral HPLC (Chiralpak AD-H column (4.6×250 mm, 5 μm), mobile phase: 20% EtOH/80% hexane, 0.8 mL/min, RT): 39.6% and 60.4% at max plot. ¹H NMR (400 MHz, DMSO-d6): 6.93 (s, 1H), 6.92 (s, exchanged with D₂O; 1H), 4.32-4.26 (m, 1H), 4.17-4.10 (m, 1H), 3.72 (s, 3H), 3.36-3.29 (m, 1H), 3.21-3.14 (m, 1H), 2.60-2.54 (m, 1H), 1.79-1.71 (m, 1H), 1.28 (s, 9H), 1.12-1.07 (m, 6H).

### Step 6

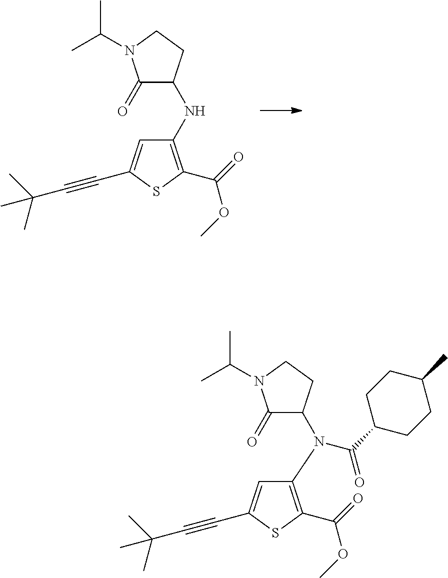 [SMILES: COC(=O)c1sc(C#CC(C)(C)C)cc1NC1CCN(C(C)C)C1=O ; COC(=O)c1sc(C#CC(C)(C)C)cc1N(C(=O)[C@H]1CC[C@H](C)CC1)C1CCN(C(C)C)C1=O]

### Methyl 5-(3,3-dimethylbut-1-ynyl)-3-[(1-isopropyl-2-oxo-pyrrolidin-3-yl)-(trans-4-methylcyclohexanecarbonyl)amino]thiophene-2-carboxylate

To a stirred solution of methyl 5-(3,3-dimethylbut-1-ynyl)-3-[(1-isopropyl-2-oxo-pyrrolidin-3-yl)amino]thiophene-2-carboxylate (260 mg, 0.720 mmol), pyridine (4.0 mL, 20 vol), and DMAP (43 mg, 0.36 mmol) in dichloroethane (10 mL) at 0° C., a solution of 4-methylcylohexanecarbonyl chloride (1.155 g, 7.202 mmol) in dichloroethane (5 mL) was added dropwise and the mixture stirred at 100° C. for 16 h. Reaction progress was monitored by TLC. Upon completion the reaction mixture was diluted with EtOAc (50 mL), washed with 2N HCl (10 mL), 10% NaHCO₃ solution (3×20 mL), water (10 mL), brine (20 mL), dried over Na₂SO₄, and concentrated. The residue was purified (silica gel column chromatography, 100-200 mesh, 60% EtOAc in petroleum ether as eluent) to afford methyl 5-(3,3-dimethylbut-1-ynyl)-3-[(1-isopropyl-2-oxo-pyrrolidin-3-yl)-(trans-4-methylcyclohexanecarbonyl)amino]thiophene-2-carboxylate (240 mg, 68%, off-white solid). TLC: 50% EtOAc in petroleum ether, R_{f}: 0.3. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, flow rate: 0.4 mL/min, sample diluent: MeOH): MH⁺=487.5. t_{R}=2.28 min. Chiral HPLC (Chiralpak AD-H column (4.6×250 mm, 5 μm), mobile phase: 5% EtOH (0.1% DEA)/95% hexane (0.1% DEA), 0.8 mL/min, RT): 41.2% and 58.8% at 300 nm. ¹H NMR (400 MHz, DMSO-d): 7.18 (s, 0.4H), 7.00 (s, 0.6H), 5.46 (t, J=9.2 Hz; 0.6H), 4.16-4.13 (m, 0.4H), 4.09-4.04 (m, 1H), 3.77 (s, 3H), 3.16-3.10 (m, 1H), 2.98-2.96 (m, 1H), 2.25-2.21 (m, 1H), 2.11-1.97 (m, 2H), 1.59-1.52 (m, 4H), 1.46-1.41 (m, 2H), 1.28 (s, 9H).

### Step 7

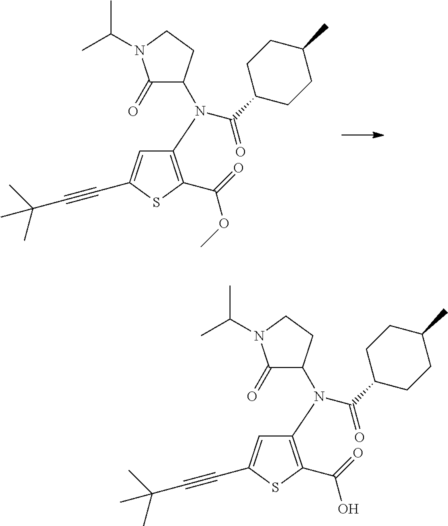

### 5-(3,3-Dimethylbut-1-ynyl)-3-[[(3R)-1-isopropyl-2-oxo-pyrrolidin-3-yl]-(trans-4-methylcyclohexanecarbonyl)amino]thiophene-2-carboxylic acid

To a stirred solution of methyl 5-(3,3-dimethylbut-1-ynyl)-3-[(1-isopropyl-2-oxo-pyrrolidin-3-yl)-(trans-4-methylcyclohexanecarbonyl)amino]thiophene-2-carboxylate (240 mg, 0.493 mmol) in 1:1 THF/water (6 mL), LiOH.H₂O (62.2 mg, 1.48 mmol) was added at RT and the reaction stirred for 3 h. Reaction progress was monitored by TLC. Upon completion, the reaction was acidified to pH˜1 using 1M aq. HCl, extracted with EtOAc (50 mL), washed with water (3×20 mL), brine (20 mL), dried over Na₂SO₄, and concentrated. The crude product was purified by chiral preparative HPLC with ethanol/hexane as eluent to give 141, 5-(3,3-dimethylbut-1-ynyl)-3-[[(3R)-1-isopropyl-2-oxo-pyrrolidin-3-yl]-(trans-4-methylcyclohexanecarbonyl)amino]thiophene-2-carboxylic acid (65 mg, 70%), TLC: 10% MeOH in CHCl₃, R_{f}: 0.4. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, 3/3, flow rate: 0.4 mL/min, sample diluent: MeOH): MH⁺=473.2. t_{R}=1.39 min. Chiral HPLC (Chiralpak IA column (4.6×250 mm, 5 μm), mobile phase: 5% EtOH (0.1% TFA)/5% i-PrOH (0.1% TFA)/90% hexane (0.1% TFA), 0.7 mL/min, RT): 99.9% at 299 nm. ¹H NMR (400 MHz, DMSO-d6): 13.6 (br s, exchanged with D₂O; 1H), 7.13 (s, 0.48H, 6.96 (s, 1H), 5.48 (t, J=9 Hz; 1H), 4.11-4.07 (m, 3H), 3.34-3.31 (m, 0.57H), 3.16-3.10 (m, 1.62H), 2.98-2.93 (m, 1H), 2.32-1.96 (m, 4H), 1.60-1.43 (m, 8H), 1.29 (s, 9H), 1.05-1.01 (m, 6H).

### Preparation of Compound 142

### Steps 1-6

### As Described Above for the Preparation of Compound 141

### Step 7

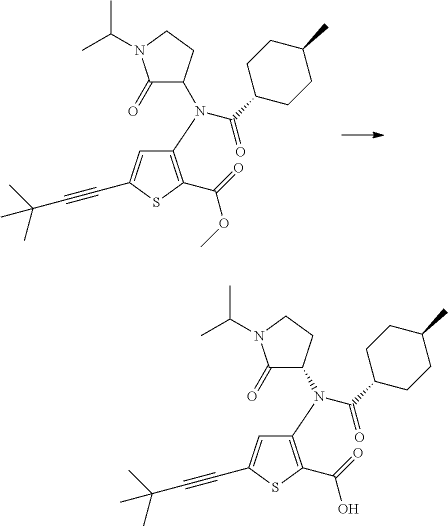

### 5-(3,3-dimethylbut-1-ynyl)-3-[[(3S)-1-isopropyl-2-oxo-pyrrolidin-3-yl]-(trans-4-methylcyclohexanecarbonyl)amino]thiophene-2-carboxylic acid

Chiral preparative HPLC of product mixture from Step 7, Compound 141 above, gave 142, 5-(3,3-dimethylbut-1-ynyl)-3-[[(3S)-1-isopropyl-2-oxo-pyrrolidin-3-yl]-(trans-4-methylcyclohexanecarbonyl)amino]thiophene-2-carboxylic acid (100 mg, 70%), TLC: 10% MeOH in CHCl₃, R_{f}: 0.4. Analysis by LCMS (column: BEH C-18 (2.1×50 mm, 1.7 μm), mobile phase: A: 5 mM aq. CH₃CO₂NH₄, B: MeCN, time (min)/% B gradient: 0 min/3%, 0.1/3, 1.5/90, 2.1/90, 2.3/100, 2.8/50, 3/3, flow rate: 0.4 mL/min, sample diluent: MeOH): MH⁺=473.2, t_{R=1.39} min. Chiral purity (Chiralpak IA column (4.6×250 mm, 5 μm), mobile phase: 5% EtOH (0.1% TFA)/5% i-PrOH (0.1% TFA)/90% hexane (0.1% TFA), 0.7 mL/min, RT): 99.9% at 299 nm. ¹H NMR (400 MHz, DMSO-d6): 13.6 (br s, exchanged with D₂O; 1H), 7.13 (s, 0.48H, 6.96 (s, 1H), 5.48 (t, J=9 Hz; 1H), 4.11-4.07 (m, 3.67H), 3.36-3.32 (m, 0.83H), 3.16-3.10 (m, 1.8H), 2.97-2.93 (m, 1H), 2.32-1.96 (m, 4H), 1.60-1.43 (m, 8H), 1.29 (s, 9H), 1.05-1.01 (m).

### Preparation of Compound 144

### Step 1

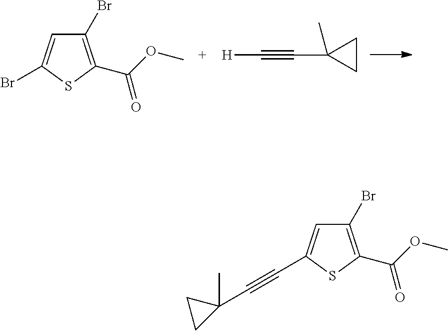

### Methyl 3-bromo-5-[2-(1-methylcyclopropyl)ethynyl]thiophene-2-carboxylate

A suspension of CuI (71 mg, 0.38 mmol) in 1,4-dioxane (40 mL) was deoxygenated by purging with argon gas for 30 min at RT, then Pd(PPh₃)₂Cl₂ (263 mg, 0.375 mmol) was added and purging continued an additional 15 min. Diisopropylamine (3.56 mL, 25.0 mmol) and methyl 3,5-dibromothiophene-2-carboxylate (3.75 g, 12.5 mmol) were added and the reaction stirred at RT for 15 min. The mixture was cooled in ice-water and 1-ethynyl-1-methylcyclopropane (ether solution, ˜1 g, 12.5 mmol) was added and the reaction stirred at RT for 1 h. Reaction progress was monitored by TLC. The mixture was then diluted with EtOAc (20 mL), filtered through celite, and the filter cake washed with EtOAc (2×20 mL). The combined filtrate was concentrated, the residue purified by preparative HPLC using MeCN and aq. HCOOH as eluent to afford methyl 3-bromo-5-[2-(1-methylcyclopropyl)ethynyl]thiophene-2-carboxylate (350 mg, pale yellow solid). TLC: 2% EtOAc in petroleum ether, R_{f}: 0.5. Analysis by LCMS (column: BEH C-18 (2.1×50 mm, 1.7 μm), mobile phase: A: 0.1% aq. HCOOH, B: MeCN, time (min)/% B gradient: 0 min/3%, 0.1/3, 1.5/90, 1.8/90, 2.2/95, 3.2/95, 3.8/3, flow rate: 0.4 mL/min, sample diluent: MeOH): MH⁺=301.2, t_{R=2.11} min. ¹H NMR (400 MHz, CDCl₃): 7.01 (s, 1H), 3.87 (s, 3H), 1.34 (s, 3H), 1.04-1.02 (m, 2H), 0.75-0.72 (m, 2H).

### Step 2

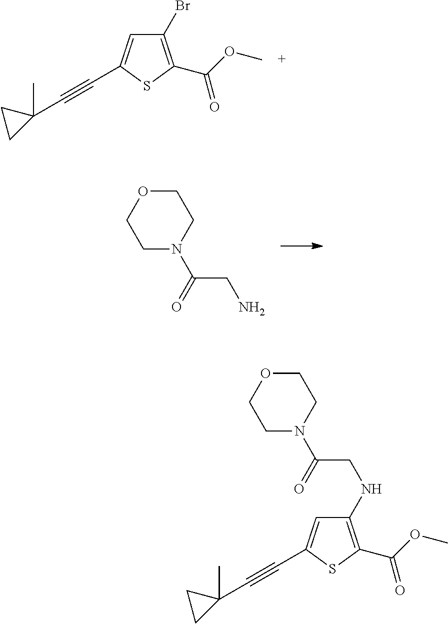 [SMILES: COC(=O)c1sc(C#CC2(C)CC2)cc1Br ; NCC(=O)N1CCOCC1 ; COC(=O)c1sc(C#CC2(C)CC2)cc1NCC(=O)N1CCOCC1]

### Methyl 5-[2-(1-methylcyclopropyl)ethynyl]-3-[(2-morpholino-2-oxo-ethyl)amino]thiophene-2-carboxylate

A suspension of methyl 3-bromo-5-[2-(1-methylcyclopropyl)ethynyl]thiophene-2-carboxylate (350 mg, 1.17 mmol), 2-amino-1-morpholino-ethanone.HCl (316 mg, 1.84 mmol), and Cs₂CO₃ (1.14 g, 3.68 mmol) in toluene (10 mL) was deoxygenated by bubbling with argon for 50 min, then Pd(OAc)₂ (26 mg, 0.12 mmol) and (±) BINAP (72 mg, 0.12 mmol) were added. Purging was continued for another 10 min and the mixture stirred at 100° C. for 16 h. Reaction progress was monitored by TLC. Upon completion, the reaction mixture was cooled to RT, diluted with EtOAc (50 mL), and filtered through celite. The filtrate was washed with water (2×20 mL), brine (30 mL), dried over Na₂SO₄, and concentrated. The residue was purified (silica gel column chromatography, 100-200 mesh, 50% EtOAc in petroleum ether as eluent) to afford methyl 5-[2-(1-methylcyclopropyl)ethynyl]-3-[(2-morpholino-2-oxo-ethyl)amino]thiophene-2-carboxylate (220 mg, 52%, yellow solid). TLC: 60% EtOAc in petroleum ether, R_{f}: 0.4. MH⁺=363.0. ¹H NMR (400 MHz, DMSO-d6): 7.23 (s, exchanged with D₂O; 1H), 6.96 (s, 1H), 4.12 (d, J=4.4 Hz; 2H), 3.71 (s, 3H), 3.60-3.56 (m, 4H), 3.48-3.45 (m, 4H), 1.30 (s, 3H), 0.98-0.95 (m, 2H), 0.80-0.79 (m, 2H).

### Step 3

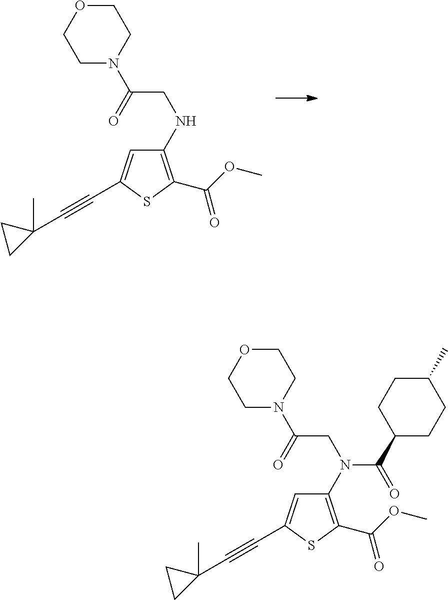 [SMILES: COC(=O)c1sc(C#CC2(C)CC2)cc1NCC(=O)N1CCOCC1 ; COC(=O)c1sc(C#CC2(C)CC2)cc1N(CC(=O)N1CCOCC1)C(=O)[C@H]1CC[C@H](C)CC1]

### Methyl 3-[(trans-4-methylcyclohexanecarbonyl)-(2-morpholino-2-oxo-ethyl)amino]-5-[2-(1-methylcyclopropyl)ethynyl]thiophene-2-carboxylate

To a solution of methyl 5-[2-(1-methylcyclopropyl)ethynyl]-3-[(2-morpholino-2-oxo-ethyl)amino]thiophene-2-carboxylate (220 mg, 0.607 mmol), Et₃N (1.27 mL, 9.10 mmol) in CH₂Cl₂ (5.0 mL) at 0° C., a solution of trans 4-methylcyclohexyl carbonyl chloride (864 mg, 6.08 mmol) in CH₂Cl₂ (10.0 mL) was added dropwise and the reaction stirred at RT for 16 h. Reaction progress was monitored by TLC. Upon completion, the rmixture was diluted with EtOAc (50 mL), washed with 2N aq. HCl (2×10 mL), 10% NaHCO₃ solution (20 mL), water (10 mL), brine (20 mL), dried over Na₂SO₄, and concentrated. The residue was purified (silica gel column chromatography, 100-200 mesh, 50% EtOAc in petroleum ether as eluent) to afford methyl 3-[(trans-4-methylcyclohexanecarbonyl)-(2-morpholino-2-oxo-ethyl)amino]-5-[2-(1-methylcyclopropyl)ethynyl]thiophene-2-carboxylate (260 mg, 88%, off-white solid). TLC: 60% EtOAc in petroleum ether, R_{f}: 0.3. Analysis by LCMS (column: BEH C-18 (2.1×50 mm, 1.7 μm), mobile phase: A: 5 mM CH₃CO₂NH₄, B: MeCN, time (min)/% B gradient: 0 min/3%, 1.5/45, 2.5/45, 3.2/95, 4.7/95, 5/3, flow rate: 0.4 mL/min, sample diluent: MeOH): MH⁺=487.1, t_{R} 3.72 min. ¹H NMR (400 MHz, DMSO-d6): 7.23 (s, 1H), 4.91 (d, J=16.8 Hz; 1H), 3.83 (d, J=16.4 Hz; 1H), 3.77 (s, 3H), 3.54-3.53 (m, 4H), 3.42-3.39 (m, 4H), 2.09-2.04 (m, 1H), 1.70-1.34 (m, 5H), 1.31 (s, 3H), 1.30-1.23 (m, 2H), 1.03-1.01 (m, 2H), 0.82-0.78 (m, 2H), 0.77 (d, J=6.4 Hz; 3H).

### Step 4

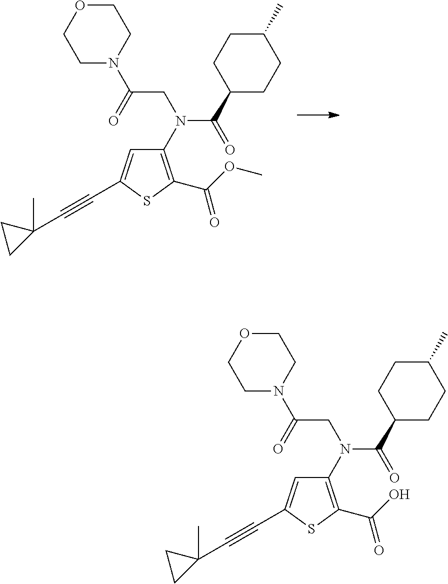 [SMILES: COC(=O)c1sc(C#CC2(C)CC2)cc1N(CC(=O)N1CCOCC1)C(=O)[C@H]1CC[C@H](C)CC1 ; CC1(C#Cc2cc(N(CC(=O)N3CCOCC3)C(=O)[C@H]3CC[C@H](C)CC3)c(C(=O)O)s2)CC1]

### 3-[(trans-4-methylcyclohexanecarbonyl)-(2-morpholino-2-oxo-ethyl)amino]-5-[2-(1-methylcyclopropyl)ethynyl]thiophene-2-carboxylic acid

To a stirred solution of methyl 3-[(trans-4-methylcyclohexanecarbonyl)-(2-morpholino-2-oxo-ethyl)amino]-5-[2-(1-methylcyclopropyl)ethynyl]thiophene-2-carboxylate (260 mg, 0.534 mmol) in 1:1 THF/water (6 mL), LiOH.H₂O (67.3 mg, 1.60 mmol) was added at RT and the reaction stirred for 16 h. Reaction progress was monitored by TLC. Upon completion, the mixture was acidified (pH˜1) with 1M aq. HCl and extracted with EtOAc (50 mL). The organic layer was washed with water (2×10 mL), brine (10 mL), dried over Na₂SO₄, and concentrated to afford 144, 3-[(trans-4-methylcyclohexanecarbonyl)-(2-morpholino-2-oxo-ethyl)amino]-5-[2-(1-methylcyclopropyl)ethynyl]thiophene-2-carboxylic acid (55 mg, 21%, off-white solid). TLC: 10% MeOH in CHCl₃, R_{f}: 0.4. Analysis by LCMS (column: BEH C-18 (2.1×50 mm, 1.7 μm), mobile phase: A: 5 mM CH₃CO₂NH₄, B: MeCN, time (min)/% B gradient: 0 min/3%, 1.5/45, 2.5/45, 3.2/95, 4.7/95, 5/3, flow rate: 0.4 mL/min, sample diluent: MeOH): MH⁺=471.5, t_{R} 2.06 min. ¹H NMR (400 MHz, DMSO-d6): 13.45 (s, exchanged with D₂O; 1H), 7.21 (s, 1H), 4.91 (d, J=16.4 Hz; 1H), 3.84 (d, J=16.4 Hz; 1H), 3.54-3.53 (m, 4H), 3.39-3.32 (m, 4H), 2.11-1.98 (m, 1H), 1.60-1.51 (m, 4H), 1.37-1.34 (m, 2H), 1.31 (s, 3H), 1.19-1.17 (m, 2H), 1.02-1.00 (m, 2H).

### Preparation of Compound 151

### Step 1

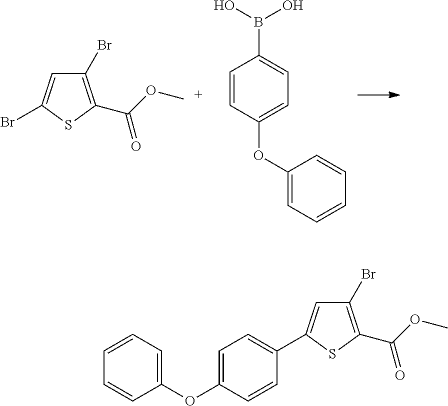

### Methyl 3-bromo-5-(4-phenoxyphenyl)thiophene-2-carboxylate

To a solution of methyl 3,5-dibromothiophene carboxylate (2.0 g, 6.7 mmol) and 4-phenoxyphenylboronic acid (1.43 g, 6.66 mmol) in toluene (60 mL), 3M aq. K₂CO₃ solution (7 mL) was added. The mixture was deoxygenated by bubbling with argon for 1 h, after which Pd(PPh₃)₄ (0.74 g, 0.66 mmol) was added and purging continued another min. The reaction was then stirred at 90° C. for 3 h. Reaction progress was monitored by TLC. Upon completion, the mixture was diluted with EtOAc (150 mL), washed with water (3×50 mL), brine (30 mL), dried over Na₂SO₄, filtered, and concentrated. The residue was purified (silica gel column chromatography, 100-200 mesh, 5-7% EtOAc-petroleum ether) to afford methyl 3-bromo-5-(4-phenoxyphenyl)thiophene-2-carboxylate (1.2 g, 46%, white solid). TLC: 5% EtOAc in petroleum ether, R_{f}: 0.4. Analysis by LCMS (column: BEH C-18 (2.1×50 mm, 1.7 μm), mobile phase: A: 0.1% aq. HCOOH, B: MeCN, time (min)/% B gradient: 0 min/3%, 0.1/3, 1.5/90, 1.8/90, 2.2/95, 3.2/95, 3.8/3, flow rate: 0.4 mL/min, sample diluent: MeOH): MH⁺=389.2, t_{R=2.27} min. ¹H NMR (400 MHz, CDCl₃): 7.55-7.53 (m, 2H), 7.40-7.36 (m, 2H), 7.21 (s, 1H), 7.18-7.16 (m, 1H), 7.07-7.01 (m, 4H), 3.91 (s, 3H).

### Step 2

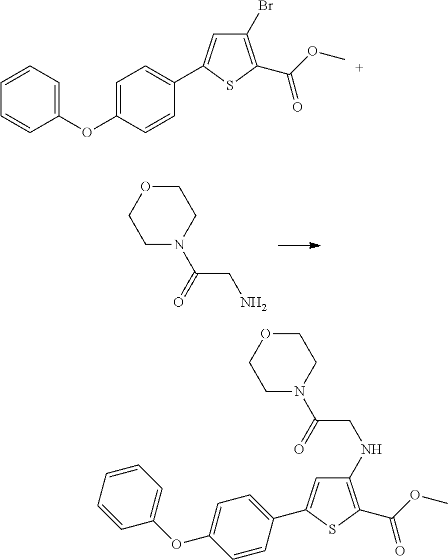

### Methyl 3-[(2-morpholino-2-oxo-ethyl)amino]-5-(4-phenoxyphenyl)thiophene-2-carboxylate

A suspension of methyl 3-bromo-5-(4-phenoxyphenyl)thiophene-2-carboxylate (1.2 g, 3.0 mmol), 2-amino-1-morpholinoethanone.HCl (720 mg, 4.0 mmol), and Cs₂CO₃ (3.01 g, 9.24 mmol) in toluene (20 mL) was deoxygenated by bubbling with argon for 60 min, then Pd(OAc)₂ (70 mg, 0.31 mmol) and (±) BINAP (389 mg, 0.660 mmol) were added. Purging was continued for 30 min then the reaction was stirred at 100° C. for 16 h. Reaction progress was monitored by TLC. Upon completion the mixture was cooled to RT, diluted with EtOAc (100 mL), and filtered through celite. The filtrate was washed with water (2×40 mL), brine (30 mL), dried over Na₂SO₄, and concentrated. The residue was purified (silica gel column chromatography, 100-200 mesh, 65% EtOAc in petroleum ether as eluent) to afford methyl 3-[(2-morpholino-2-oxo-ethyl)amino]-5-(4-phenoxyphenyl)thiophene-2-carboxylate (1.2 g, 86%, brown solid). TLC: 60% EtOAc in petroleum ether, R_{f}: 0.25. Analysis by LCMS (column: BEH C-18 (2.1×50 mm, 1.7 μm), mobile phase: A: 0.1% aq. HCOOH, B: MeCN, time (min)/% B gradient: 0 min/3%, 0.1/3, 1.5/90, 1.8/90, 2.2/95, 3.2/95, 3.8/3, flow rate: 0.4 mL/min, sample diluent: MeOH): MH⁺=453.4, t_{R}=1.95 min. ¹H NMR (400 MHz, CDCl₃): 7.58-7.55 (m, 2H), 7.43 (br s, exchanged with D₂O; 1H), 7.38-7.35 (m, 2H), 7.17-7.13 (m, 1H), 7.05-6.99 (m, 4H), 6.72 (s, 1H), 4.11 (d, J=5.2 Hz; 2H), 3.85 (s, 3H), 3.72-3.69 (m, 6H), 3.49-3.48 (m, 2H).

### Step 3

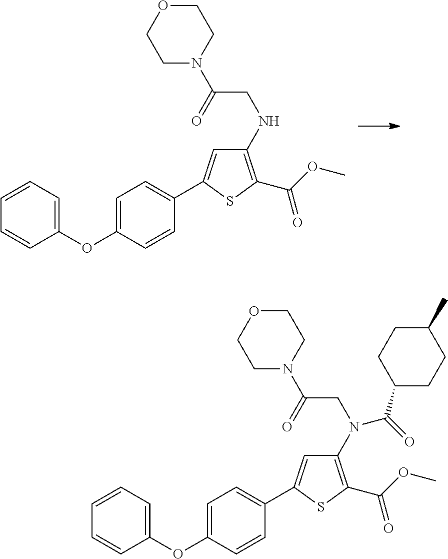 [SMILES: COC(=O)c1sc(-c2ccc(Oc3ccccc3)cc2)cc1NCC(=O)N1CCOCC1 ; COC(=O)c1sc(-c2ccc(Oc3ccccc3)cc2)cc1N(CC(=O)N1CCOCC1)C(=O)[C@H]1CC[C@H](C)CC1]

### Methyl 3-[(trans-4-methylcyclohexanecarbonyl)-(2-morpholino-2-oxo-ethyl)amino]-5-(4-phenoxyphenyl)thiophene-2-carboxylate

To a solution of methyl 3-[(2-morpholino-2-oxo-ethyl)amino]-5-(4-phenoxyphenyl)thiophene-2-carboxylate (300 mg, 0.66 mmol) and Et₃N (1.4 mL, 9.9 mmol) in CH₂Cl₂ (5 mL) at 0° C., trans 4-methylcyclohexyl carbonyl chloride (1.06 g, 6.60 mmol) in CH₂Cl₂ (5 mL) was added dropwise and the reaction stirred at RT for 16 h. Reaction progress was monitored by TLC. Upon completion, the mixture was diluted with EtOAc (100 mL), washed with 2N aq. HCl (2×20 mL), water (20 mL), 10% NaHCO₃ solution (2×20 mL), brine (20 mL), dried over Na₂SO₄, and concentrated. The residue was purified (silica gel column chromatography, 100-200 mesh, 50% EtOAc in petroleum ether as eluent) to afford methyl 3-[(trans-4-methylcyclohexanecarbonyl)-(2-morpholino-2-oxo-ethyl)amino]-5-(4-phenoxyphenyl)thiophene-2-carboxylate (300 mg, 79%, white solid). TLC: 50% EtOAc in petroleum ether, R_{f}: 0.35. Analysis by LCMS (column: Purospher RP-18 (2.0×100 mm, 2.1 μm), mobile phase: A: 5 mM CH₃CO₂NH₄, B: MeCN, time (min)/% B gradient: 0 min/3%, 1.5/45, 2.5/45, 3.2/95, 4.7/95, 5/3, flow rate: 0.6 mL/min, sample diluent: MeOH): MH⁺=577.1, t_{R}=4.12 min. ¹H NMR (400 MHz, CDCl₃): 7.61-7.59 (m, 2H), 7.49 (s, 1H), 7.40-7.36 (m, 2H), 7.18-7.15 (m, 1H), 7.06-7.02 (m, 4H), 5.30-5.21 (m, 2H), 3.87 (s, 3H), 3.70-3.45 (m, 100H), 2.25-2.19 (m, 1H), 1.87-1.84 (m, 1H), 1.67-1.60 (m, 3H), 1.33-1.26 (m, 2H), 0.79 (d, J=6.4 Hz).

### Step 4

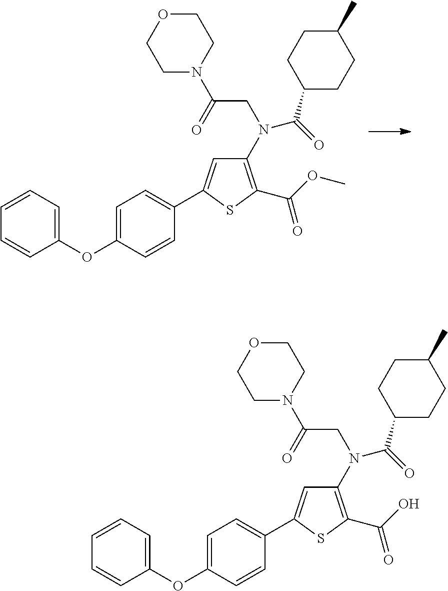 [SMILES: COC(=O)c1sc(-c2ccc(Oc3ccccc3)cc2)cc1N(CC(=O)N1CCOCC1)C(=O)[C@H]1CC[C@H](C)CC1 ; C[C@H]1CC[C@H](C(=O)N(CC(=O)N2CCOCC2)c2cc(-c3ccc(Oc4ccccc4)cc3)sc2C(=O)O)CC1]

### 3-[(trans-4-methylcyclohexanecarbonyl)-(2-morpholino-2-oxo-ethyl)amino]-5-(4-phenoxyphenyl)thiophene-2-carboxylic acid

To a stirred solution of methyl 3-[(trans-4-methylcyclohexanecarbonyl)-(2-morpholino-2-oxo-ethyl)amino]-5-(4-phenoxyphenyl)thiophene-2-carboxylate (300 mg, 0.52 mmol) in 1:1 THF/water (20 mL), LiOH.H₂O (65.5 mg, 1.56 mmol) was added at RT and the reaction stirred for 16 h. Reaction progress was monitored by TLC. Upon completion, the mixture was acidified (pH˜1) with 1M aq. HCl and extracted with EtOAc (60 mL). The organic layer was washed with water (3×20 mL), brine (20 mL), dried over Na₂SO₄, and concentrated to afford 151, 3-[(trans-4-methylcyclohexanecarbonyl)-(2-morpholino-2-oxo-ethyl)amino]-5-(4-phenoxyphenyl)thiophene-2-carboxylic acid (110 mg, 38%, white solid). TLC: 10% MeOH in CHCl₃, R_{f}: 0.32. Analysis by LCMS (column: BEH C-18 (2.1×50 mm, 1.7 μm), mobile phase: A: 5 mM CH₃CO₂NH₄, B: MeCN, time (min)/% B gradient: 0 min/3%, 1.5/45, 2.5/45, 3.2/95, 4.7/95, 5/3, flow rate: 0.6 mL/min, sample diluent: MeOH): MH⁺=563.0, t_{R}=2.43 min. ¹H NMR (400 MHz, DMSO-d6): 13.3 (br s, exchanged with D₂O; 1H), 7.75-7.73 (m, 2H), 7.50 (s, 1H), 7.45-7.41 (m, 2H), 7.22-7.18 (m, 1H), 7.09-7.07 (4H), 4.95 (d, J=16.4 Hz; 1H), 3.92 (d, J=15.6 Hz; 1H), 3.55-3.36 (m, 8H), 2.22-2.16 (m, 1H), 1.75-1.72 (m, 1H), 1.60.

### Preparation of Compound 152

### Steps 1-2

### As Described Above for the Preparation of Compound 151

### Step 3

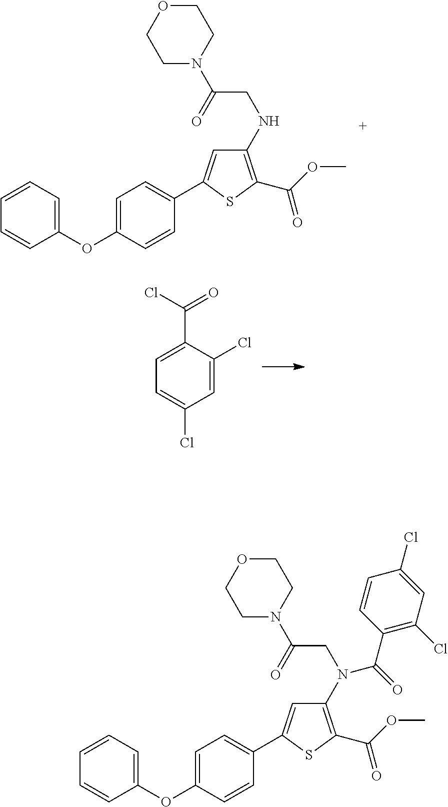

### Methyl 3-[(2,4-dichlorobenzoyl)-(2-morpholino-2-oxo-ethyl)amino]-5-(4-phenoxyphenyl)thiophene-2-carboxylate

To a solution of methyl 3-[(2-morpholino-2-oxo-ethyl)amino]-5-(4-phenoxyphenyl)thiophene-2-carboxylate (300 mg, 0.66 mmol) and Et₃N (1.4 mL, 9.9 mmol) in CH₂Cl₂ (5 mL) at 0° C., a solution of 2,4-dichlorobenzoyl chloride (0.92 mL, 6.6 mmol) in CH₂Cl₂ (5 mL) was added dropwise and the reaction stirred at RT for 16 h. Reaction progress was monitored by TLC. Upon completion, the mixture was diluted with EtOAc (100 mL), washed with 2N aq. HCl (2×20 mL), water (20 mL), 10% NaHCO₃ solution (2×20 mL), brine (20 mL), dried over Na₂SO₄, and concentrated. The residue was purified (silica gel column chromatography, 100-200 mesh, 55% EtOAc in petroleum ether as eluent) to afford methyl 3-[(2,4-dichlorobenzoyl)-(2-morpholino-2-oxo-ethyl)amino]-5-(4-phenoxyphenyl)thiophene-2-carboxylate (330 mg, 80%, white solid). TLC: 50% EtOAc in petroleum ether, R_{f}: 0.35. Analysis by LCMS (column: Purospher RP-18 (2.0×100 mm, 2.1 μm), mobile phase: A: mM CH₃CO₂NH₄, B: MeCN, time (min)/% B gradient: 0 min/3%, 1.5/45, 2.5/45, 3.2/95, 4.7/95, 5/3, flow rate: 0.6 mL/min, sample diluent: MeOH): MH⁺=626.8, t_{R}=3.97 min. ¹H NMR (400 MHz, CDCl₃): 7.41-7.35 (m, 5H), 7.27-7.26 (m, 1H), 7.22-7.14 (m, 2H), 7.10-7.08 (m, 2H), 6.98-6.95 (m, 2H), 5.39 (br, 1H), 4.09 (br, 1H), 3.90 (s, 3H), 3.70-3.58 (m, 8H).

### Step 4

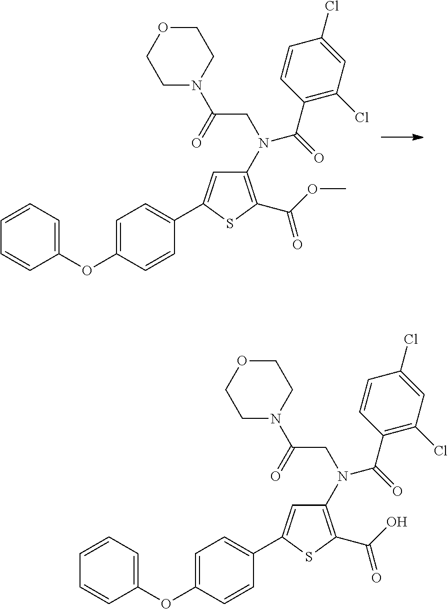 [SMILES: COC(=O)c1sc(-c2ccc(Oc3ccccc3)cc2)cc1N(CC(=O)N1CCOCC1)C(=O)c1ccc(Cl)cc1Cl ; O=C(O)c1sc(-c2ccc(Oc3ccccc3)cc2)cc1N(CC(=O)N1CCOCC1)C(=O)c1ccc(Cl)cc1Cl]

### 3-[(2,4-dichlorobenzoyl)-(2-morpholino-2-oxo-ethyl)amino]-5-(4-phenoxyphenyl)thiophene-2-carboxylic acid

To a stirred solution of methyl 3-[(2,4-dichlorobenzoyl)-(2-morpholino-2-oxo-ethyl)amino]-5-(4-phenoxyphenyl)thiophene-2-carboxylate (320 mg, 0.52 mmol) in 1:1 of THF/water (20 mL), LiOH.H₂O (65.4 mg, 1.55 mmol) was added at RT and the reaction stirred for 16 h. Reaction progress was monitored by TLC. Upon completion the mixture was acidified (pH˜1) with 1M aq. HCl and extracted with EtOAc (60 mL). The organic layer was washed with water (3×20 mL), brine (20 mL), dried over Na₂SO₄, and concentrated. The crude residue was triturated with Et₂O (2×5 mL) to afford 152, 3-[(2,4-dichlorobenzoyl)-(2-morpholino-2-oxo-ethyl)amino]-5-(4-phenoxyphenyl)thiophene-2-carboxylic acid (120 mg, 38%, white solid). TLC: 10% MeOH in CHCl₃, R_{f}: 0.3. Analysis by LCMS (column: BEH C-18 (2.1×100 mm, 1.7 μm), mobile phase: A: 5 mM aq. CH₃CO₂NH₄, B: MeCN, time (min)/% B gradient: 0 min/3%, 1.5/45, 2.5/45, 3.2/95, 4.7/95, 5/3, flow rate: 0.6 mL/min, sample diluent: MeOH): MH⁺=610.8. t_{R}=2.37 min. ¹H NMR (400 MHz, DMSO-d6): 13.5 (br s, exchanged with D₂O; 1H), 7.57-7.53 (m, 3H), 7.44-7.36 (m, 4H), 7.31-7.29 (m, 1H), 7.21-7.17 (m, 1H), 7.08-7.03 (m, 4H), 5.19-5.15 (m, 1H), 4.31-4.27 (m, 1H), 3.60-3.45 (m, 8H).

### Preparation of Compounds 93 and 94

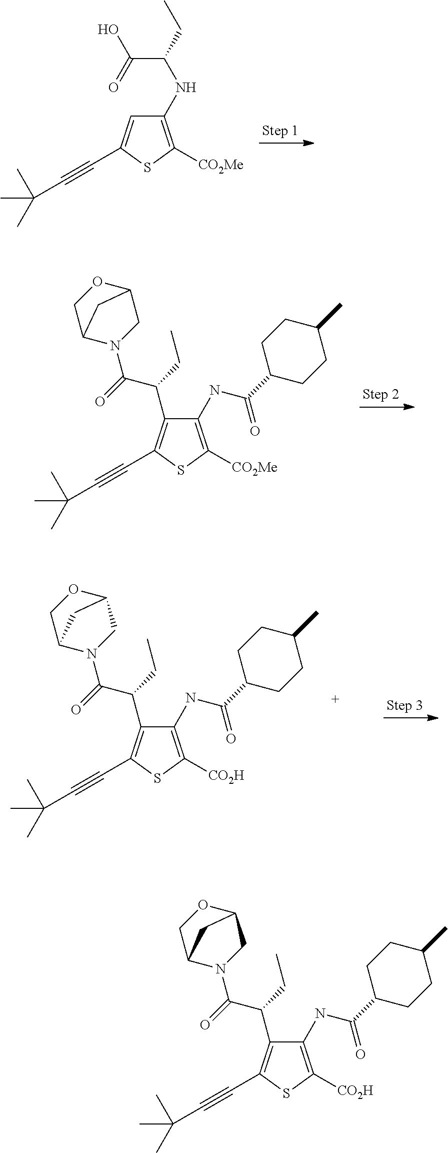

### Step 1

### Methyl 5-(3,3-dimethylbut-1-ynyl)-3-[(trans-4-methylcyclohexanecarbonyl)-[(1S)-1-[(3-oxa-6-azabicyclo[2.2.1]heptane-6-carbonyl]propyl]amino]thiophene-2-carboxylate

To a solution of (2S)-2-[[5-(3,3-dimethylbut-1-ynyl)-2-methoxycarbonyl-3-thienyl]-(trans-4-methylcyclohexanecarbonyl)amino]butanoic acid, prepared as described for compound 82, (200 mg, 0.45 mmol) in DMF (5 mL) was added diisopropylethylamine (311 μL, 1.79 mmol), HBTU (339 mg, 0.89 mmol) and 6-oxa-3-azabicyclo[2.2.1]heptane hydrochloride (121 mg, 0.89 mmol). The mixture was stirred for 2 h, then diluted with EtOAc. The solution was washed with H₂O and brine. The solvent was evaporated and the product purified by silica gel column chromatography, eluting with 0-50% EtOAc in hexane to afford the desired product, methyl 5-(3,3-dimethylbut-1-ynyl)-3-[(trans-4-methylcyclohexanecarbonyl)-[(1S)-1-[(3-oxa-6-azabicyclo[2.2.1]heptane-6-carbonyl]propyl]amino]thiophene-2-carboxylate.

### Step 2

The ester obtained above was dissolved in MeOH (3 mL) and a 1N solution of NaOH (1.34 mL) added. The solution was stirred overnight, then neutralized to pH=3 with 1N HCl. The product was extracted (EtOAc) and the solution concentrated. The two isomeric products were purified by silica gel column chromatography, eluting with 0-3% MeOH in CH₂Cl₂. The material (90 mg) was separated by supercritical fluid chromatography (Whelk-O eluted with 8% MeOH/TFA, CO₂) into the two isomers, compounds 93 and 94. Structural assignment was made by specific synthesis of compound 94. Compound 93: MS: m/z (obs.) 515.0 [M+H]⁺. Compound 94: MS: m/z (obs.) 515.0 [M+H]⁺; ¹H NMR (300 MHz, CDCl₃) δ 7.67-7.49 (d, 1H), 6.79-6.67 (d, 1H); 5.23-4.98 (m, 1H), 4.85-4.61 (m, 2H), 4.42 (ddd, 1H), 4.11 (dd, 1H), 3.88-3.74 (m, 2H); 3.49 (dd, 2H); 2.14-1.82 (m, 3H); 1.77-1.39 (m, 7H), 1.39-1.27 (m, 9H); 1.04-0.54 (m, 8H).

### Preparation of Compound 121

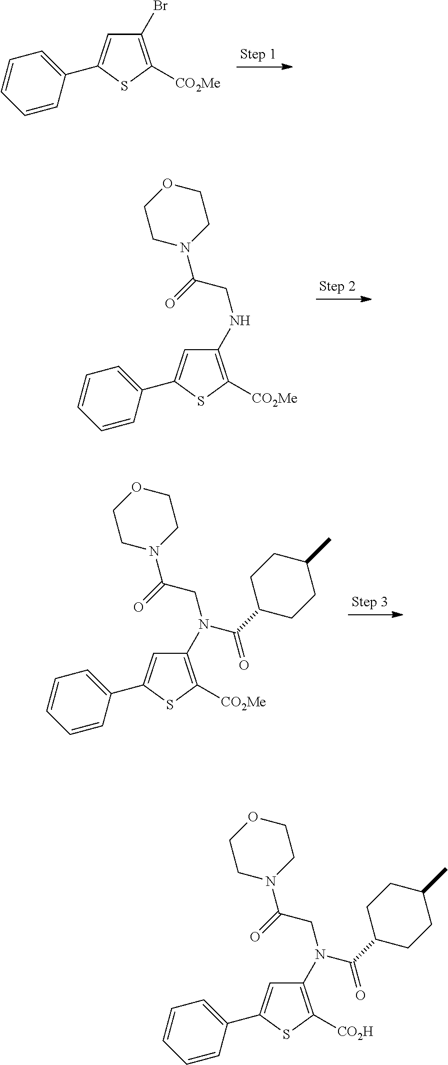 [SMILES: COC(=O)c1sc(-c2ccccc2)cc1Br ; COC(=O)c1sc(-c2ccccc2)cc1NCC(=O)N1CCOCC1 ; COC(=O)c1sc(-c2ccccc2)cc1N(CC(=O)N1CCOCC1)C(=O)[C@H]1CC[C@H](C)CC1 ; C[C@H]1CC[C@H](C(=O)N(CC(=O)N2CCOCC2)c2cc(-c3ccccc3)sc2C(=O)O)CC1]

### Step 1

### Methyl 3-((2-morpholino-2-oxoethyl)amino)-5-phenylthiophene-2-carboxylate

A suspension of methyl 3-bromo-5-phenylthiophene-2-carboxylate (375 mg, 1.27 mmol), 2-amino-1-morpholinoethanone hydrochloride (344 mg, 1.91 mmol), Cs₂CO₃ (1.24 g, 3.81 mmol) in toluene (10 mL) was deoxygenated by bubbling with argon for 1 h, and Pd(OAc)₂ (29 mg, 0.13 mmol), (±)-BINAP (79 mg, 0.13 mmol) were added. The purging was continued for another 30 min and the mixture stirred at 100° C. for 16 h. Reaction progress was monitored by TLC. On completion the reaction mixture was cooled to RT, diluted with EtOAc (100 mL) and filtered through celite. The filtrate was washed with water (2×35 mL), brine (30 mL), dried over Na₂SO₄, and concentrated. The residue was purified by column chromatography (100-200 mesh silica gel) using 30% EtOAc in pet ether as eluant to afford methyl 3-((2-morpholino-2-oxoethyl)amino)-5-phenylthiophene-2-carboxylate (200 mg, 43.6%, white solid). TLC system: 50% EtOAc in pet ether, Rf: 0.38. MS: m/z (obs.) 361.0 [M+H]⁺. 1H NMR (400 MHz, CDCl₃) δ 7.73-7.71 (m, 2H), 7.49-7.40 (m, 3H), 7.35-7.33 (m, 2H), 4.25 (d, 2H), 3.75 (s, 3H), 3.65-3.58 (m, 4H), 3.50-3.49 (m, 4H).

### Step 2

### Methyl 3-[(trans-4-methylcyclohexanecarbonyl)-N-(2-morpholino-2-oxoethyl)amino]-5-phenylthiophene-2-carboxylate

To a solution of methyl 3-((2-morpholino-2-oxoethyl)amino)-5-phenylthiophene-2-carboxylate (200 mg, 0.55 mmol), Et₃N (0.76 mL, 5.55 mmol) in CH₂Cl₂ (5 mL) at 0° C., a solution of trans 4-methylcyclohexylcarbonyl chloride (883 mg, 5.5 mmol) in CH₂Cl₂ (5 mL) was added drop wise and stirred at RT for 16 h. Reaction progress was monitored by TLC. On completion, the reaction mixture was diluted with EtOAc (80 mL), washed with 2N aq HCl (2×30 mL), water (30 mL), 10% NaHCO3 solution (2×30 mL), brine (30 mL), dried over Na₂SO₄ and concentrated. The residue was purified by column chromatography (100-200 mesh silica gel) using 50% EtOAc in pet ether as eluent to afford methyl 3-[(trans-4-methylcyclohexanecarbonyl)-N-(2-morpholino-2-oxoethyl)amino]-5-phenylthiophene-2-carboxylate (100 mg, 37.4%, white solid). TLC system: 50% EtOAc in pet ether. Rf: 0.35. MS: m/z (obs.) 485.2 [M+H]⁺. 1H NMR (400 MHz, CDCl₃) δ 7.76-7.74 (m, 2H), 7.61 (s, 1H), 7.51-7.45 (m, 3H), 4.97 (d, 1H), 3.91 (d, 1H), 3.81 (s, 3H), 3.55-3.39 (m, 8H), 2.18 (br s, 1H), 1.78-1.75 (m, 1H), 1.58-1.50 (m, 3H), 1.40-1.31 (m, 3H), 0.75 (d, 3H).

### Step 3

### 3-[(trans-4-Methylcyclohexanecarbonyl)-N-(2-morpholino-2-oxoethyl)amino]-5-phenylthiophene-2-carboxylic acid

To a solution of methyl 3-[(trans-4-methylcyclohexanecarbonyl)-N-(2-morpholino-2-oxoethyl)amino]-5-phenylthiophene-2-carboxylate (100 mg, 0.206 mmol) in 1:1 mixture of THF and water (4 mL), LiOH monohydrate (26 mg, 0.62 mmol) was added at RT and stirred for 16 h. The reaction progress was monitored by TLC. On completion, the reaction mixture was acidified (pH˜1) with 1M aq HCl, extracted with EtOAc (60 mL). The organic layer was washed with water (3×20 mL), brine (20 mL), dried over Na₂SO₄ and concentrated to afford 121, 3-[(trans-4-methylcyclohexanecarbonyl)-N-(2-morpholino-2-oxoethyl)amino]-5-phenylthiophene-2-carboxylic acid (70 mg, 72%, white solid). TLC system: 10% MeOH in CHCl₃. Rf: 0.31. MS: m/z (obs.) 469.0 [M+H]⁺. 1H NMR (400 MHz, CDCl₃) δ 13.42 (br s, exchanged with D₂O; 1H), 7.73-7.72 (m, 2H), 7.57-7.41 (m, 4H), 4.96 (d, 1H), 3.93 (d, 1H), 3.55-3.40 (m, 8H), 2.22-2.16 (m, 1H), 1.75-1.72 (m, 1H), 1.57 (br s, 2H), 1.42-1.23 (m, 4H), 0.75 (d, J=6 Hz).

### Preparation of Compound 153

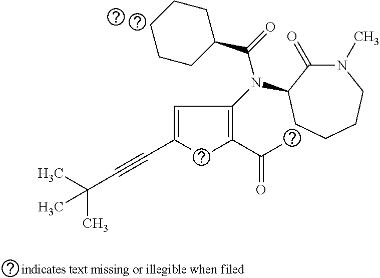

Compound 153 was prepared in a similar manner as that described for Compound 95. MS: m/z (obs.) 473.3 [M+H]⁺. 1H NMR (300 MHz, CDCl₃) δ 7.61 (s, 0.6H), 6.75 (s, 0.4H), 5.30 (br s, 0.6H), 5.08 (d, 0.4H), 4.00-3.61 (m, 1H), 3.39-3.13 (m, 1H), 3.09 (s, 1.2H), 3.05 (s, 1.8H), 2.23-1.39 (m, 14H), 1.34 (two singlets, 9H), 0.75 (m, 5H).

### Preparation of Compound 154

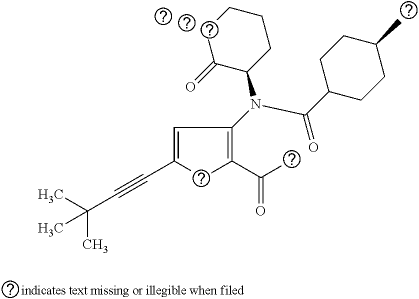

Compound 154 was isolated from a sample of Compound 117 by chiral chromatography on a Chiralpak-IC column eluted with 0.1% TFA in MeCN. MS: m/z (obs.) 459.4 [M+H]⁺. 1H NMR (300 MHz, CDCl₃) δ 6.84 (s, 1H), 3.80-3.48 (m, 2H), 3.36-3.15 (m, 1H), 3.05 (s, 3H), 2.65-2.35 (m, 1H), 2.35-1.93 (m, 4H), 1.93-1.75 (m, 2H), 1.74-1.45 (m, 4H), 1.35 (s, 9H), 0.84 (d, 3H), 0.77-0.63 (m, 1H).

### Preparation of Compound 10

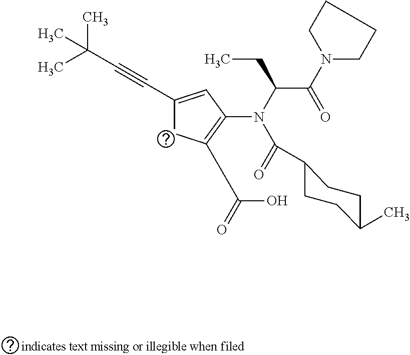 [SMILES: CC[C@@H](C(=O)N1CCCC1)N(C(=O)C1CCC(C)CC1)C1=C(C(=O)O)CC(C#CC(C)(C)C)=C1]

Prepared according to the procedure for compound 14. Analysis carried out by LCMS (LCMS method m1 17:60-98% MeOH; 5/7 min(grad/run) with formic acid modifier, 7 minutes, (C18)); RT=5.56 min, MH+=487.48 (strong).

### Preparation of Compound 11

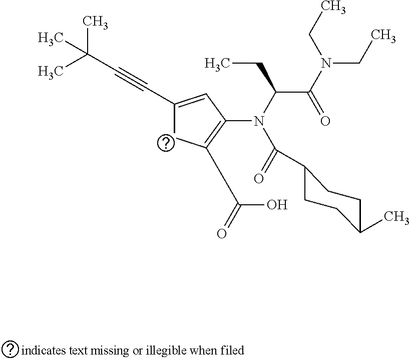 [SMILES: CC[C@@H](C(=O)N(CC)CC)N(C(=O)C1CCC(C)CC1)C1=C(C(=O)O)CC(C#CC(C)(C)C)=C1]

Prepared according to the procedure for compound 14. Analysis carried out by LCMS (LCMS method m117: 60-98% MeOH; 5/7 min (grad/run) with formic acid modifier, 7 minutes, (C18)); RT=5.5 min, MD+=487.5 (strong).

### Preparation of Compound 17

### Step 1

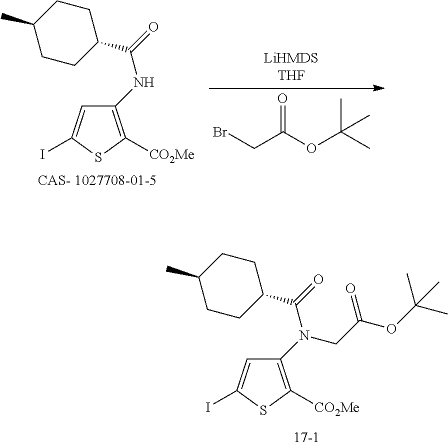

### Methyl 3-((1r,4r)-N-(2-(tert-butoxy)-2-oxoethyl)-4-methylcyclohexanecarboxamido)-5-iodothiophene-2-carboxylate

In a dry flask added amide (CAS—1027708-01-5) (1 g, 2.333 mmol) and THF (9.500 mL), and placed under nitrogen. Cooled with an ice bath. Added (bis(trimethylsilyl)amino)lithium (4.0 mL of 1 M, 2.916 mmol), and stirred at 0° C. for 25 minutes. Added tert-butyl 2-bromoacetate (850 mg, 4.1 mmol), and allowed reaction to warm to ambient temperature and then stirred at 50° C. for 24 hours. Aqueous work-up was carried out partitioning between brine and ethyl acetate to obtain 1.6 g. Purified by flash chromatography on silica gel. Eluted using an ethyl acetate and hexane mixture 0-20% over 100CV's then at 20% ethyl acetate, for 2CV's. Obtained pure fractions, combined and removed solvent to obtain 0.6 g of solid 17-1, methyl 3-((1r,4r)-N-(2-(tert-butoxy)-2-oxoethyl)-4-methylcyclohexanecarboxamido)-5-iodothiophene-2-carboxylate (49% of theory). Analysis carried out by LCMS (LCMS method m117:60-98% MeOH; 5/7 min(grad/run) with formic acid modifier, 7 minutes, (C18)); RT=5.42 min, MH+=522.16 (strong).

### Step 2

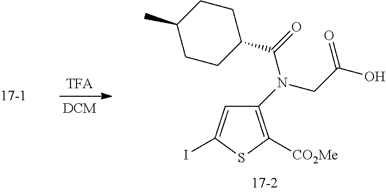 [SMILES: COC(=O)c1sc(I)cc1N(CC(=O)O)C(=O)[C@H]1CC[C@H](C)CC1]

### 2-((1r,4r)-N-(5-iodo-2-(methoxycarbonyl)thiophen-3-yl)-4-methylcyclohexanecarboxamido)acetic acid

Compound 17-1 (3.7 g, 6.741 mmol) was dissolved in DCM (35 mL), and cooled to 0° C. using an external ice-bath. To this solution was added TFA (15 mL, 194.7 mmol; 30% (TFA/DCM solution), allowed to come to RT slowly as ice bath melts/ON. After stirring overnight, test the reaction by HPLC—starting material is consumed. The solvent was removed under vacuum to give 2 g of a reddish brown glass 17-2 2-((1r,4r)-N-(5-iodo-2-(methoxycarbonyl)thiophen-3-yl)-4-methylcyclohexanecarboxamido)acetic acid (51%) of thory. Analysis was carried out by LCMS (LCMS method m117:60-98% MeOH; 5/7 min(grad/run) with formic acid modifier, 7 minutes, (C18)); RT=3.43 min, MH+=466.11 (strong).

### Step 3

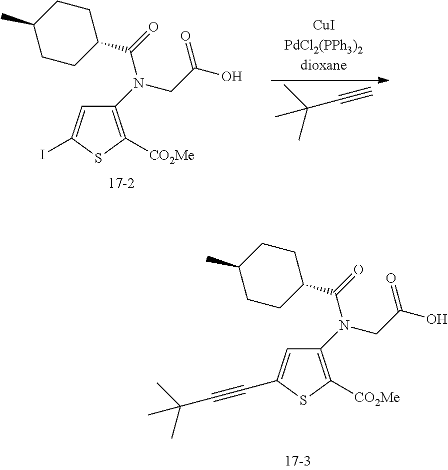

### 2-((1r,4r)-N-(5-(3,3-dimethylbut-1-yn-1-yl)-2-(methoxycarbonyl)thiophen-3-yl)-4-methylcyclohexanecarboxamido)acetic acid

In a dry 100 mL flask under nitrogen atmosphere, mixed 17-2 (2.5 g, 4.315 mmol), in dioxane (54 mL), and cooled with an external ice bath and blew nitrogen over the reaction. Added iodocopper (41.18 mg, 0.2162 mmol), followed by 3,3-dimethylbut-1-yne (800 μL, 6.700 mmol) and bubbled nitrogen for 5 min. Added dichloro-bis(triphenylphosphoranyl)palladium (164.9 mg, 0.2343 mmol), and then the bath was removed and the reaction was stirred at RT overnight. The reaction was homogeneous; and shown to be complete by tlc (20% MeOH/DCM). Purified by Adding EtOAC (50 mL), and filter through fluoracil, rinse with EtOAc (3×25 mL), then elute with 20% MeOH/EtOAc until product is not coming off. Combine fractions and remove solvent to give a black solid. Dissolve the solid in ethyl acetate, and washed with aqueous 1N HCl, back extract the aqueous with ethyl acetate, then added decolorizing carbon and sodium sulfate, filter and remove the solvent to give 1.6 g of colorless solid 17-3,2-((1r,4r)-N-(5-(3,3-dimethylbut-1-yn-1-yl)-2-(methoxycarbonyl)thiophen-3-yl)-4-methylcyclohexanecarboxamido)acetic acid. Analysis was carried out by LCMS (LCMS method m117:60-98% MeOH; 5/7 min(grad/run) with formic acid modifier, 7 minutes, (C18)); RT=4.63 min, MH+=420.36 (strong).

### Step 4

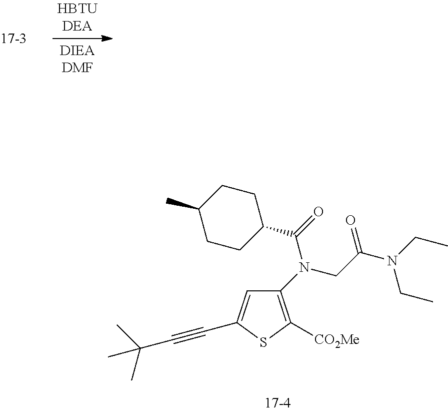 [SMILES: CCN(CC)C(=O)CN(c1cc(C#CC(C)(C)C)sc1C(=O)OC)C(=O)[C@H]1CC[C@H](C)CC1]

### Methyl 3-((1r,4r)-N-(2-(diethylamino)-2-oxoethyl)-4-methylcyclohexanecarboxamido)-5-(3,3-dimethylbut-1-yn-1-yl)thiophene-2-carboxylate

Compound 17-3 (290 mg, 0.6912 mmol) was placed in a vial along with HBTU (1.659 mL of 0.5 M, 0.8294 mmol) (0.5M solution in DMF prepared ahead of time and stored in a freezer). Added DIEA (134.0 mg, 180.6 μL, 1.037 mmol) followed by diethylamine (61.45 mg, 72.12 μL, 0.8640 mmol), and the reaction stirred at RT/ON. Checked completion of reaction by HPLC, and by LCMS. Aqueous workup with brine (60 mL), extracted with iso-propyl acetate (2×60 mL); dried over sodium sulfate, filter and strip to obtain crude product (400 mg). Purified by flash chromatography on silica gel column 12, and eluted with 0-50% ethyl acetate-Hexanes. Obtained 130 mgs as a solid product 17-4, methyl 3-((1r,4r)-N-(2-(diethylamino)-2-oxoethyl)-4-methylcyclohexanecarboxamido)-5-(3,3-dimethylbut-1-yn-1-yl)thiophene-2-carboxylate. Analysis was carried out by LCMS (LCMS method m217:60-98% Acetonitrile; 5/7 min(grad/run) with formic acid modifier, (C4)); RT=2.33 min, MH+=473.47 (strong).

### Step 5

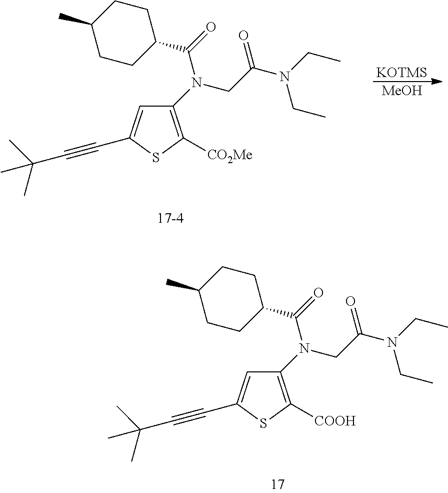

### Compound 17

Starting 17-4 (90 mg, 0.189 mmol) was mixed with 2N sodium hydroxide (5 mL, 10 mmol) and stirred overnight. The reaction using HPLC was judged complete by disappearance of starting material. Under vacuum, removed methanol. added 1N HCl (aq), 15 mL, and brine 15 mL. Extract 2×30 mL 1:1 diethyl ether-ethyl acetate (30 mL), dried over sodium sulfate, filtered and removed the solvent to give crude product. Purified using HPLC (C18, 40-95% CH3CN/H₂O; 0.1% TFA), the homogeneous fractions were combined and solvent removed under vacuum to give 66 mg solid 17. Analysis was carried out by LCMS (LCMS method m217:60-98% Acetonitrile; 5/7 min(grad/run) with formic acid modifier, (C4)); RT=1.87 min, MH+=461.44 (strong).

### Preparation of Compound 25

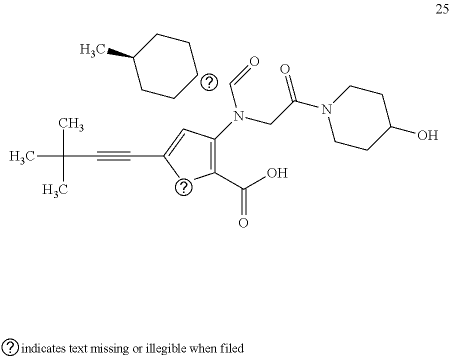

Prepared according to the procedure for compound 17. Analysis carried out by LCMS (LCMS method m208: 40-80% MeOH; 5/7 min(grad/run) with formic acid modifier, 7 minutes, (C18)); RT=3.53 min, MH+=489.1 (strong).

### Preparation of Compound 27

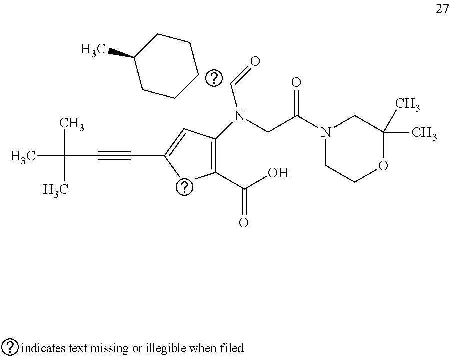

Prepared according to the procedure for compound 17. Analysis carried out by LCMS (LCMS method m208: 40-80% MeOH; 5/7 min(grad/run) with formic acid modifier, 7 minutes, (C18)); RT=4.76 min, MH+=503.10 (strong).

### Preparation of Compound 28

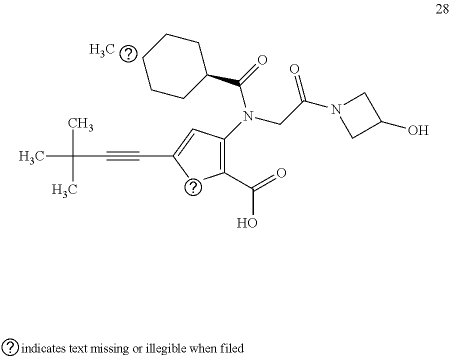

Prepared according to the procedure for compound 17. Analysis carried out by LCMS (LCMS method m208: 40-80% MeOH; 5/7 min(grad/run) with formic acid modifier, 7 minutes, (C18)); RT=3.21 min, MH+=461.06 (strong).

### Preparation of Compound 41

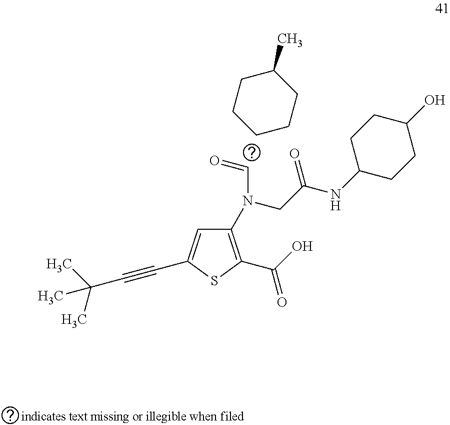

Prepared according to the procedure for compound 17. Analysis carried out by LCMS (LCMS method m208: 40-80% MeOH; 5/7 min(grad/run) with formic acid modifier, 7 minutes, (C18)); RT=4.12 min, MH+=503.10 (strong).

### Preparation of Compound 42

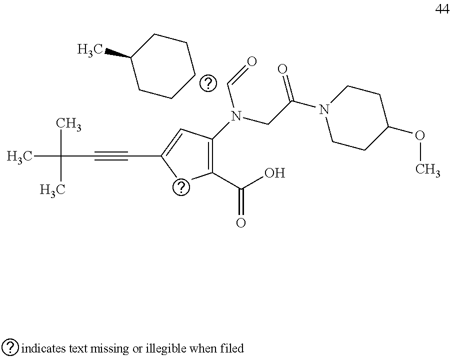

Prepared according to the procedure for compound 17. Analysis carried out by LCMS (LCMS method m217:60-98% Acetonitrile; 5/7 min(grad/run) with formic acid modifier, (C4)); RT=2.30 min, MH+=474.94 (strong).

### Preparation of Compound 43

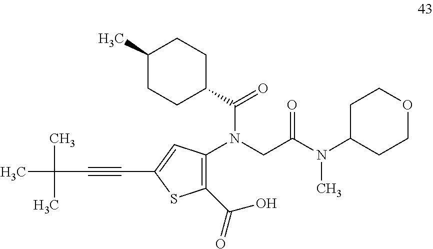 [SMILES: CN(C(=O)CN(c1cc(C#CC(C)(C)C)sc1C(=O)O)C(=O)[C@H]1CC[C@H](C)CC1)C1CCOCC1]

Prepared according to the procedure for compound 17. Analysis carried out by LCMS (LCMS method m217:60-98% Acetonitrile; 5/7 min(grad/run) with formic acid modifier, (C4)); RT=3.34 min, MH+=502.99 (strong).

### Preparation of Compound 44

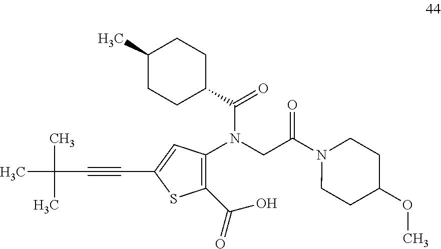 [SMILES: COC1CCN(C(=O)CN(c2cc(C#CC(C)(C)C)sc2C(=O)O)C(=O)[C@H]2CC[C@H](C)CC2)CC1]

Prepared according to the procedure for compound 17. Analysis carried out by LCMS (LCMS method m217:60-98% Acetonitrile; 5/7 min(grad/run) with formic acid modifier, (C4)); RT=3.5 min, MH+=502.99 (strong).

### Preparation of Compound 45

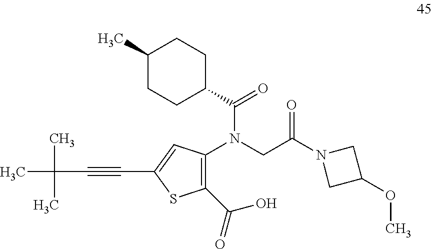 [SMILES: COC1CN(C(=O)CN(c2cc(C#CC(C)(C)C)sc2C(=O)O)C(=O)[C@H]2CC[C@H](C)CC2)C1]

Prepared according to the procedure for compound 17. Analysis carried out by LCMS (LCMS method m217:60-98% Acetonitrile; 5/7 min(grad/run) with formic acid modifier, (C4)); RT=3.8 min, MH+=474.96 (strong).

### Preparation of Compound 46

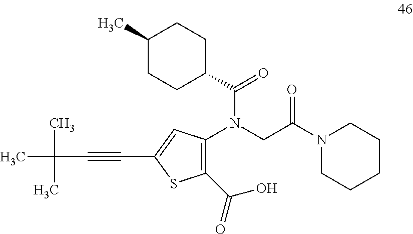 [SMILES: CC(C)(C)C#Cc1cc(N(CC(=O)N2CCCCC2)C(=O)[C@H]2CC[C@H](C)CC2)c(C(=O)O)s1]

Prepared according to the procedure for compound 17. Analysis carried out by LCMS (LCMS method m217:60-98% Acetonitrile; 5/7 min(grad/run) with formic acid modifier, (C4)); RT=3.8 min, MH+=472.62 (strong).

### Preparation of Compound 97

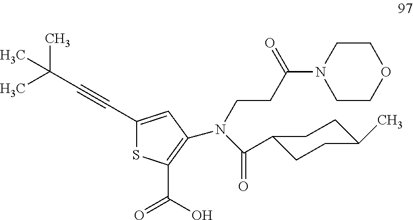 [SMILES: CC1CCC(C(=O)N(CCC(=O)N2CCOCC2)c2cc(C#CC(C)(C)C)sc2C(=O)O)CC1]

The compound was prepared according to the procedure for compound 14. Analysis carried out by LCMS (LCMS method m117:60-98% MeOH; 5/7 min(grad/run) with formic acid modifier, 7 minutes, (C18)); RT=5.30 min, MH+=489.47 (strong).

### Preparation of Compound 98

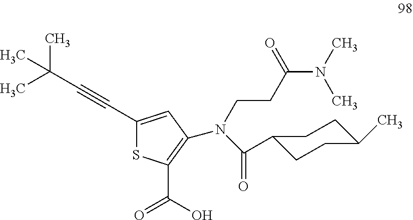 [SMILES: CC1CCC(C(=O)N(CCC(=O)N(C)C)c2cc(C#CC(C)(C)C)sc2C(=O)O)CC1]

The compound was prepared according to the procedure for compound 14. Analysis carried out by LCMS (LCMS method m117:60-98% MeOH; 5/7 min(grad/run) with formic acid modifier, 7 minutes, (C18)); RT=5.22 min, MH+=447.43 (strong).

### Preparation of Compound 96

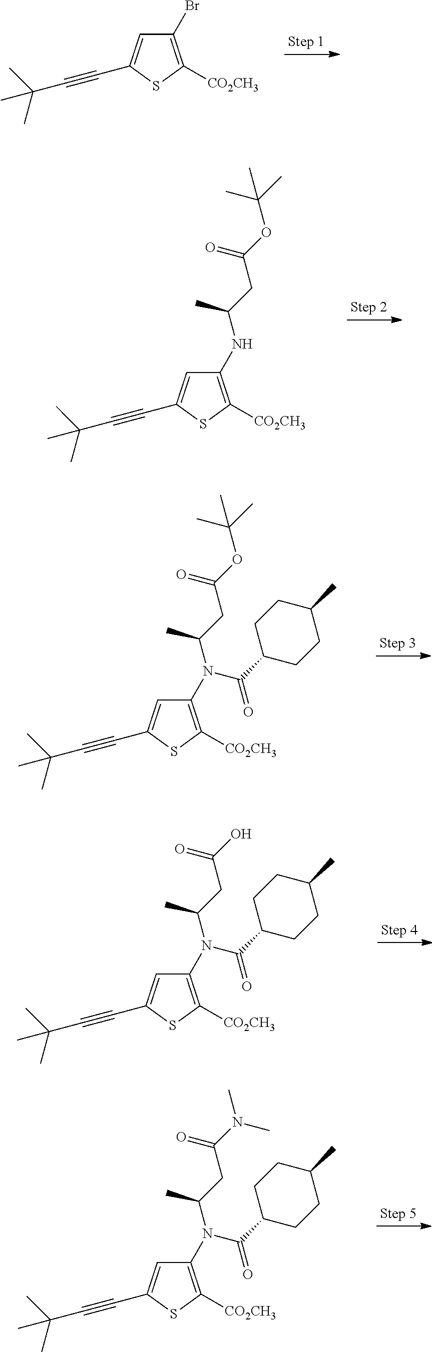

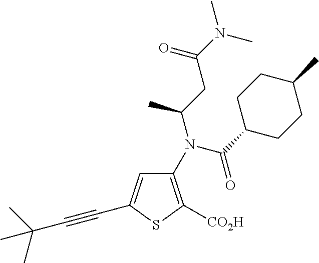 [SMILES: C[C@@H](CC(=O)N(C)C)N(c1cc(C#CC(C)(C)C)sc1C(=O)O)C(=O)[C@H]1CC[C@H](C)CC1]

### Step 1

### (S)-methyl 3-((4-(tert-butoxy)-4-oxobutan-2-yl)amino)-5-(3,3-dimethylbut-1-yn-1-yl)thiophene-2-carboxylate

A suspension of methyl 3-bromo-5-(3,3-dimethylbut-1-yn-1-yl)thiophene-2-carboxylate (4.73 g, 15.70 mmol), tert-butyl-(3S)-3-aminobutanoate (2.5 g, 15.70 mmol) and cesium carbonate (15.35 g, 47.10 mmol) in 1,4-dioxane (79 mL) was degassed with a nitrogen stream for 30 minutes. Pd₂(dba)₃ (719 mg, 0.79 mmol) and S-Phos (645 mg, 1.57 mmol) were added and the mixture was heated overnight at 90 deg C. in a sealed tube. The reaction mixture was diluted with DCM and filtered over Celite. The filtrate was concentrated and purified via flash chromatography (EtOAc/hexanes) to give (S)-methyl 3-((4-(tert-butoxy)-4-oxobutan-2-yl)amino)-5-(3,3-dimethylbut-1-yn-1-yl)thiophene-2-carboxylate (5.5 g, 92%). MS: m/z (obs): 380.3 [M+H]⁺.

### Step 2

### Methyl 3-((trans)-N—((S)-4-(tert-butoxy)-4-oxobutan-2-yl)-4-methylcyclohexanecarboxamido)-5-(3,3-dimethylbut-1-yn-1-yl)thiophene-2-carboxylate

To (S)-methyl 3-((4-(tert-butoxy)-4-oxobutan-2-yl)amino)-5-(3,3-dimethylbut-1-yn-1-yl)thiophene-2-carboxylate (5.5 g, 14.49 mmol) and trans-4-methylcyclohexanecarbonyl chloride (3.49 g, 21.74 mmol) in DCE (145 mL) was added pyridine (5.86 mL, 72.45 mmol). The reaction was stirred overnight at 90 deg C. in a sealed tube. An additional 2 g of trans-4-methylcyclohexanecarbonyl chloride were added, followed by 20 mg of DMAP. The reaction mixture was stirred again overnight at 90 deg C., diluted with DCM, then washed with 1 M HCl and brine. The organic layer was dried, concentrated and purified via flash chromatography (EtOAc/hexanes) to give methyl 3-((trans)-N—((S)-4-(tert-butoxy)-4-oxobutan-2-yl)-4-methylcyclohexanecarboxamido)-5-(3,3-dimethylbut-1-yn-1-yl)thiophene-2-carboxylate (4.5 g, 61.7%). MS: m/z (obs): 504.3 [M+H]⁺.

### Step 3

### (S)-3-((trans)-N-(5-(3,3-dimethylbut-1-yn-1-yl)-2-(methoxycarbonyl)thiophen-3-yl)-4-methylcyclohexanecarboxamido)butanoic acid

To methyl 3-((trans)-N—((S)-4-(tert-butoxy)-4-oxobutan-2-yl)-4-methylcyclohexanecarboxamido)-5-(3,3-dimethylbut-1-yn-1-yl)thiophene-2-carboxylate (4.5 g, 8.93 mmol) in DCM (18 mL) was added TFA (18 mL). The reaction mixture was stirred at room temperature for 15 minutes, concentrated under reduced pressure, co-evaporated with DCM (3×20 mL), and dried under vacuum overnight to give (S)-3-((trans)-N-(5-(3,3-dimethylbut-1-yn-1-yl)-2-(methoxycarbonyl)thiophen-3-yl)-4-methylcyclohexanecarboxamido)butanoic acid (4.7 g, quant.). MS: m/z (obs): 448.0 [M+H]⁺.

### Step 4

### Methyl 3-((trans)-N—((S)-4-(dimethylamino)-4-oxobutan-2-yl)-4-methylcyclohexanecarboxamido)-5-(3,3-dimethylbut-1-yn-1-yl)thiophene-2-carboxylate

To (S)-3-((trans)-N-(5-(3,3-dimethylbut-1-yn-1-yl)-2-(methoxycarbonyl)thiophen-3-yl)-4-methylcyclohexanecarboxamido)butanoic acid (400 mg, 0.89 mmol), EDC (257 mg, 1.34 mmol), HOBt (181 mg, 1.34 mmol) and triethylamine (249 μL, 1.79 mmol) in DCM (5 mL) was added dimethylamine (4.47 mL, 8.94 mmol, 2 M in THF). The reaction mixture was stirred overnight at room temperature then washed with water, 1 M HCl, and sodium bicarbonate. The organic layer was dried, concentrated and purified via flash chromatography (EtOAc/hexanes) to give methyl 3-((trans)-N—((S)-4-(dimethylamino)-4-oxobutan-2-yl)-4-methylcyclohexanecarboxamido)-5-(3,3-dimethylbut-1-yn-1-yl)thiophene-2-carboxylate (175 mg, 41.3%). MS: m/z (obs): 475.3 [M+H]⁺.

### Step 5

### 3-((trans)-N—((S)-4-(dimethylamino)-4-oxobutan-2-yl)-4-methylcyclohexanecarboxamido)-5-(3,3-dimethylbut-1-yn-1-yl)thiophene-2-carboxylic acid

To methyl 3-((trans)-N—((S)-4-(dimethylamino)-4-oxobutan-2-yl)-4-methylcyclohexanecarboxamido)-5-(3,3-dimethylbut-1-yn-1-yl)thiophene-2-carboxylate (170 mg, 0.36 mmol) in THF (3 mL) and water (1 mL) was added lithium hydroxide monohydrate (75 mg, 1.79 mmol). The reaction mixture was stirred overnight, acidified with 1 M HCl, and concentrated to remove organic solvent. The resulting precipitate was filtered, washed with water, and dried under vacuum to give 96, 3-((trans)-N—((S)-4-(dimethylamino)-4-oxobutan-2-yl)-4-methylcyclohexanecarboxamido)-5-(3,3-dimethylbut-1-yn-1-yl)thiophene-2-carboxylic acid (110 mg, 66.7%).

### Preparation of Compounds 101, 102, 103

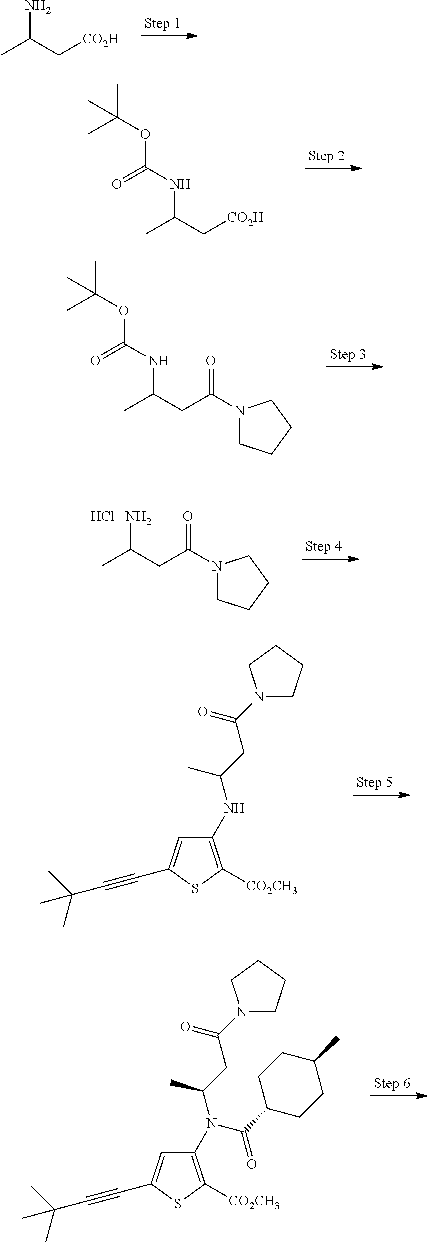

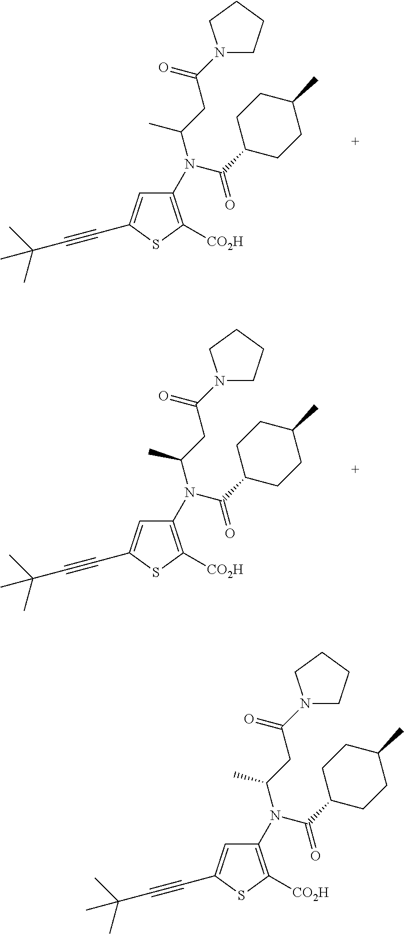

### Step 1

### 3-((Tert-butoxycarbonyl)amino)butanoic acid

To 3-aminobutanoic acid (14.4 g, 139.6 mmol) in a solution of 0.5 M aqueous sodium hydroxide and 1,4-dioxane (1:1 mixture, 460 mL) was added Boc-anhydride (30.47 g, 139.6 mmol). The reaction was stirred overnight at room temperature, concentrated to remove organic solvent, and extracted with EtOAc (2×250 mL). The combined organics were dried and concentrated to afford 3-((tert-butoxycarbonyl)amino)butanoic acid (25.3 g, 89.2%).

### Step 2

### Tert-butyl (4-oxo-4-(pyrrolidin-1-yl)butan-2-yl)carbamate

To a mixture of 3-((tert-butoxycarbonyl)amino)butanoic acid (1.65 g, 8.12 mmol), EDC (1.71 g, 8.93 mmol), and HOBt (1.21 g, 8.93 mmol) in DMF (40 mL) was added triethylamine (3.40 mL, 24.36 mmol) followed by pyrrolidine (746 L, 8.93 mmol). The reaction mixture was stirred at room temperature for several days, diluted with water (40 mL), and extracted with MTBE (3×80 mL). The combined organics were washed with 1 M HCl followed by saturated sodium bicarbonate solution, then dried and concentrated to give tert-butyl (4-oxo-4-(pyrrolidin-1-yl)butan-2-yl)carbamate (760 mg, 36.5%).

### Step 3

### 3-Amino-1-(pyrrolidin-1-yl)butan-1-one hydrochloride

Tert-butyl (4-oxo-4-(pyrrolidin-1-yl)butan-2-yl)carbamate (760 mg, 2.96 mmol) was treated with a solution of HCl in 1,4-dioxane (5 mL, 4.0 M) and stirred overnight at room temperature. The reaction mixture was concentrated to dryness to give 3-amino-1-(pyrrolidin-1-yl)butan-1-one (570 mg, quant.) as the hydrochloride salt.

### Step 4

### Methyl 5-(3,3-dimethylbut-1-yn-1-yl)-3-((4-oxo-4-(pyrrolidin-1-yl)butan-2-yl)amino)thiophene-2-carboxylate

A suspension of methyl 3-bromo-5-(3,3-dimethylbut-1-yn-1-yl)thiophene-2-carboxylate (782 mg, 2.60 mmol), 3-amino-1-(pyrrolidin-1-yl)butan-1-one hydrochloride (500 mg, 2.60 mmol) and cesium carbonate (2.54 g, 7.79 mmol) in 1,4-dioxane (13 mL) was degassed with a nitrogen stream for 30 minutes. Pd₂(dba)₃ (119 mg, 0.13 mmol) and S-Phos (107 mg, 0.26 mmol) were added and the reaction mixture was heated in a sealed tube at 90 deg C. for 48 hours. The crude reaction mixture was filtered over Celite, concentrated to dryness, and purified via flash chromatography (EtOAc/hexanes) to provide methyl 5-(3,3-dimethylbut-1-yn-1-yl)-3-((4-oxo-4-(pyrrolidin-1-yl)butan-2-yl)amino)thiophene-2-carboxylate (420 mg, 43.0%). MS: m/z (obs): 377.3 [M+H]⁺.

### Step 5

### Methyl 5-(3,3-dimethylbut-1-yn-1-yl)-3-((trans)-4-methyl-N-(4-oxo-4-(pyrrolidin-1-yl)butan-2-yl)cyclohexanecarboxamido)thiophene-2-carboxylate

To a solution of methyl 5-(3,3-dimethylbut-1-yn-1-yl)-3-((4-oxo-4-(pyrrolidin-1-yl)butan-2-yl)amino)thiophene-2-carboxylate (420 mg, 1.12 mmol) in DCE (7 mL) was added trans-4-methylcyclohexanecarbonyl chloride (179 mg, 1.12 mmol) followed by pyridine (90 L, 1.12 mmol). The reaction mixture was stirred overnight at 90 deg C., diluted with DCM, and washed with water. The organic layer was dried, concentrated, and purified via flash chromatography (EtOAc/hexanes followed by MeOH/DCM) to give methyl 5-(3,3-dimethylbut-1-yn-1-yl)-3-((trans)-4-methyl-N-(4-oxo-4-(pyrrolidin-1-yl)butan-2-yl)cyclohexanecarboxamido)thiophene-2-carboxylate (210 mg, 37.6%). MS: m/z (obs): 501.4 [M+H]⁺.

### Step 6

### Compounds 101, 102, 103

To a solution of methyl 5-(3,3-dimethylbut-1-yn-1-yl)-3-((trans)-4-methyl-N-(4-oxo-4-(pyrrolidin-1-yl)butan-2-yl)cyclohexanecarboxamido)thiophene-2-carboxylate (210 mg, 0.42 mmol) in THF (3 mL) and water (1 mL) was added lithium hydroxide monohydrate (176 mg, 4.19 mmol). The reaction mixture was stirred overnight at room temperature and acidified using 6 M HCl. The organic solvent was removed and the resulting precipitate was filtered, washed thoroughly with water, and dried to give racemic 5-(3,3-dimethylbut-1-yn-1-yl)-3-((trans)-4-methyl-N-(4-oxo-4-(pyrrolidin-1-yl)butan-2-yl)cyclohexanecarboxamido)thiophene-2-carboxylic acid 101 (121 mg, 59.3%). A portion of the racemic material (90 mg) was then separated by supercritical fluid chromatography (SFC) into the two enantiomers 102 and 103.

### Preparation of Compound 105

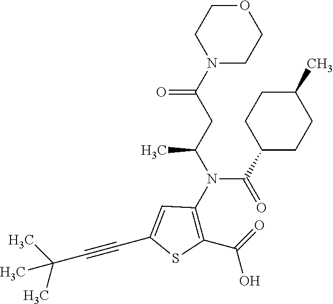 [SMILES: C[C@@H](CC(=O)N1CCOCC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-methyl-3-morpholino-3-oxo-propyl]amino]thiophene-2-carboxylic acid

Prepared according to the procedure of Compound 101.

### Preparation of Compound 106

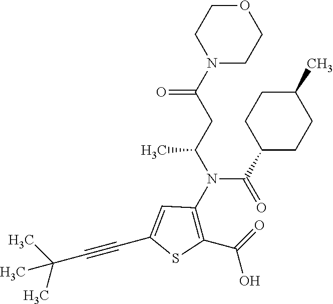 [SMILES: C[C@H](CC(=O)N1CCOCC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1R)-1-methyl-3-morpholino-3-oxo-propyl]amino]thiophene-2-carboxylic acid

Prepared according to the procedure of Compound 101.

### Preparation of Compound 110

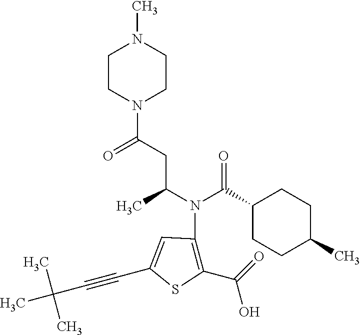 [SMILES: C[C@@H](CC(=O)N1CCN(C)CC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-methyl-3-(4-methylpiperazin-1-yl)-3-oxo-propyl]amino]thiophene-2-carboxylic acid

Prepared according to the procedure of Compound 96.

### Preparation of Compound 111

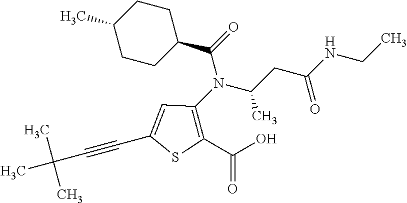 [SMILES: CCNC(=O)C[C@H](C)N(c1cc(C#CC(C)(C)C)sc1C(=O)O)C(=O)[C@H]1CC[C@H](C)CC1]

### 5-(3,3-Dimethylbut-1-ynyl)-3-[[(1S)-3-(ethylamino)-1-methyl-3-oxo-propyl]-(trans-4-methylcyclohexanecarbonyl)amino]thiophene-2-carboxylic acid

Prepared according to the procedure of Compound 96.

### Preparation of Compound 113

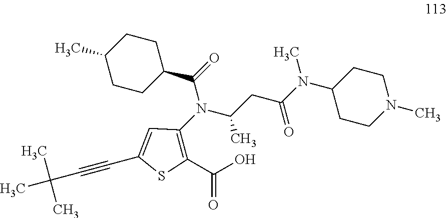 [SMILES: C[C@@H](CC(=O)N(C)C1CCN(C)CC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-methyl-3-[methyl-(1-methyl-4-piperidyl)amino]-3-oxo-propyl]amino]thiophene-2-carboxylic acid

Prepared according to the procedure of Compound 96.

### Preparation of Compound 114

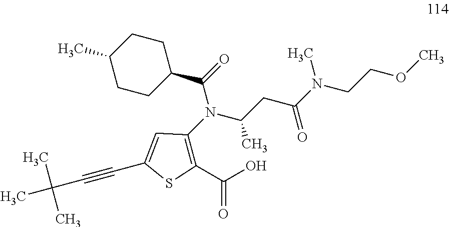 [SMILES: COCCN(C)C(=O)C[C@H](C)N(c1cc(C#CC(C)(C)C)sc1C(=O)O)C(=O)[C@H]1CC[C@H](C)CC1]

### 5-(3,3-Dimethylbut-1-ynyl)-3-[[(1S)-3-[2-methoxyethyl(methyl)amino]-1-methyl-3-oxo-propyl]-(trans-4-methylcyclohexanecarbonyl)amino]thiophene-2-carboxylic acid

Prepared according to the procedure of Compound 96.

### Preparation of Compound 115

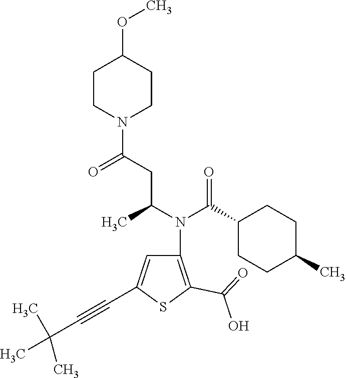 [SMILES: COC1CCN(C(=O)C[C@H](C)N(c2cc(C#CC(C)(C)C)sc2C(=O)O)C(=O)[C@H]2CC[C@H](C)CC2)CC1]

### 5-(3,3-Dimethylbut-1-ynyl)-3-[[(1S)-3-(4-methoxy-1-piperidyl)-1-methyl-3-oxo-propyl]-(trans-4-methylcyclohexanecarbonyl)amino]thiophene-2-carboxylic acid

Prepared according to the procedure of Compound 96.

### Preparation of Compound 116

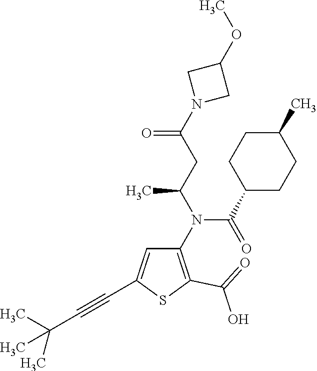 [SMILES: COC1CN(C(=O)C[C@H](C)N(c2cc(C#CC(C)(C)C)sc2C(=O)O)C(=O)[C@H]2CC[C@H](C)CC2)C1]

### 5-(3,3-Dimethylbut-1-ynyl)-3-[[(1S)-3-(3-methoxyazetidin-1-yl)-1-methyl-3-oxo-propyl]-(trans-4-methylcyclohexanecarbonyl)amino]thiophene-2-carboxylic acid

Prepared according to the procedure of Compound 96.

### Preparation of Compounds 21 & 22

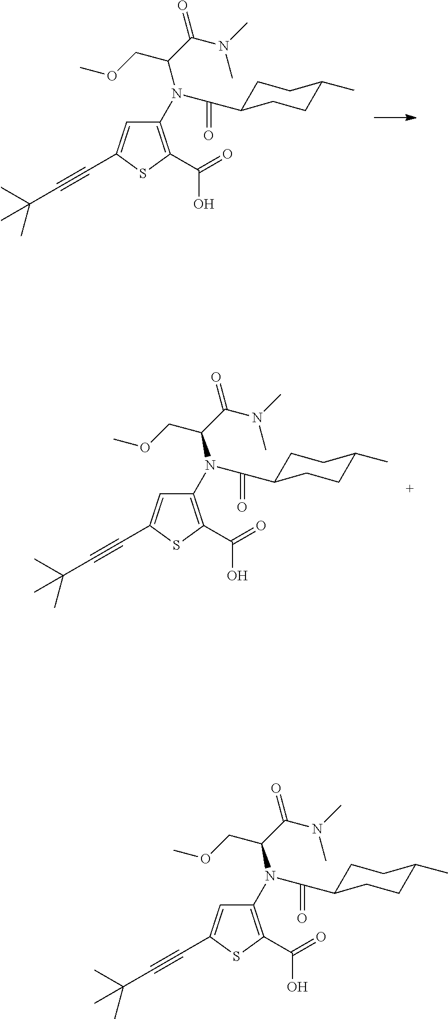

### 3-[[(1S)-2-(Dimethylamino)-1-(methoxymethyl)-2-oxo-ethyl]-(trans-4-methylcyclohexanecarbonyl)amino]-5-(3,3-dimethylbut-1-ynyl)thiophene-2-carboxylic acid and 3-[[(1R)-2-(dimethylamino)-1-(methoxymethyl)-2-oxo-ethyl]-(trans-4-methylcyclohexanecarbonyl)amino]-5-(3,3-dimethylbut-1-ynyl)thiophene-2-carboxylic acid

The two enantiomers were separated using SFC under following conditions: Column: Whelk-O (10×250); Mobile phase: 15% EtOH, 85% CO2; Flow rate: mL/min; Pressure: 100 bar.

### Preparation of Compound 24

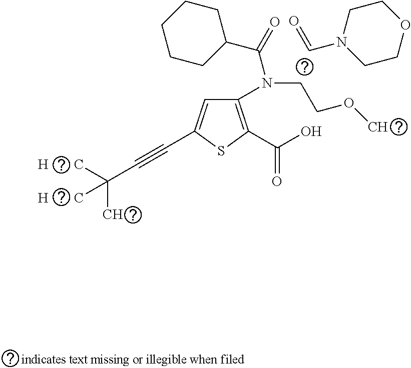

### 3-[Cyclohexanecarbonyl-[(1S)-1-(methoxymethyl)-2-morpholino-2-oxo-ethyl]amino]-5-(3,3-dimethylbut-1-ynyl)thiophene-2-carboxylic acid

Prepared according to the procedures described in compound 23. LCMS: 353.3 (MH+). 1H NMR (300 MHz, DMSO) δ 13.43 (s, 1H), 7.28 (s, 0.5H), 7.07 (s, 0.5H), 5.83 (t, J=7.2 Hz, 0.5H), 5.41 (t, J=6.7 Hz, 0.5H), 3.79-2.89 (m, 14H), 2.11-1.00 (m, 17H), 0.87-0.39 (m, 4H).

### Preparation of Compound 36

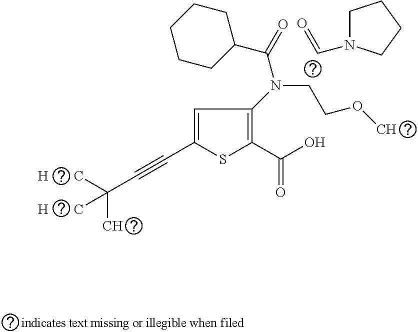

### 3-[Cyclohexanecarbonyl-[(1S)-1-(methoxymethyl)-2-oxo-2-pyrrolidin-1-yl-ethyl]amino]-5-(3,3-dimethylbut-1-ynyl)thiophene-2-carboxylic acid

Prepared according to the procedures described in compound 23. LCMS: 489.34 (MH+). ¹H NMR (300 MHz, DMSO) δ 13.35 (brs, 1H), 7.34 (s, 0.4H), 7.02 (s, 0.6H), 5.60 (t, 0.4H), 5.18 (t, 0.6H), 3.77-2.81 (m, 10H), 2.13-0.80 (m, 23H).

### Preparation of Compound 63

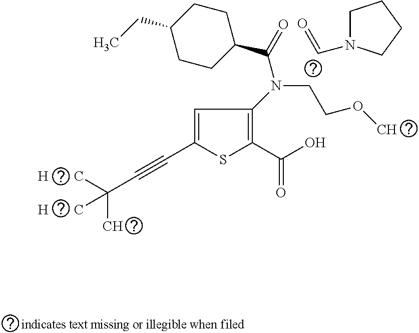

### 5-(3,3-Dimethylbut-1-ynyl)-3-[(4-ethylcyclohexanecarbonyl)-[(1S)-1-(methoxymethyl)-2-oxo-2-pyrrolidin-1-yl-ethyl]amino]thiophene-2-carb oxylic acid

Prepared according to the procedures described in compound 23. LCMS: 517.36 (MH+). 1H NMR (300 MHz, CDCl₃) δ 7.64 (s, 0.4H), 6.85 (s, 0.6H), 5.36 (dd, 0.4H), 5.02 (t, 0.6H), 4.00-3.15 (m, 10H), 2.13-0.98 (m, 24H), 0.96-0.43 (m, 4H).

### Preparation of Compound 68

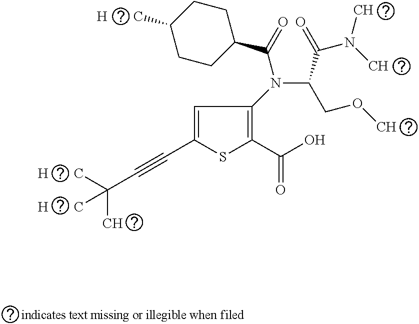

### 5-(3,3-Dimethylbut-1-ynyl)-3-[[(1S)-1-(dimethylcarbamoyl)butyl]-(trans-4-methylcyclohexanecarbonyl)amino]thiophene-2-carboxylic acid

Prepared according to the procedures described in compound 23. LCMS: 475.33 (MH+); ¹H NMR (300 MHz, CDCl₃) δ 7.60 (s, 0.4H), 6.74 (s, 0.6H), 5.32 (dd, 0.4H), 5.07 (dd, 0.6H), 3.39 (s, 3H), 3.10-2.94 (m, 4H), 2.14-1.22 (m, 29H).

### Preparation of Compound 99

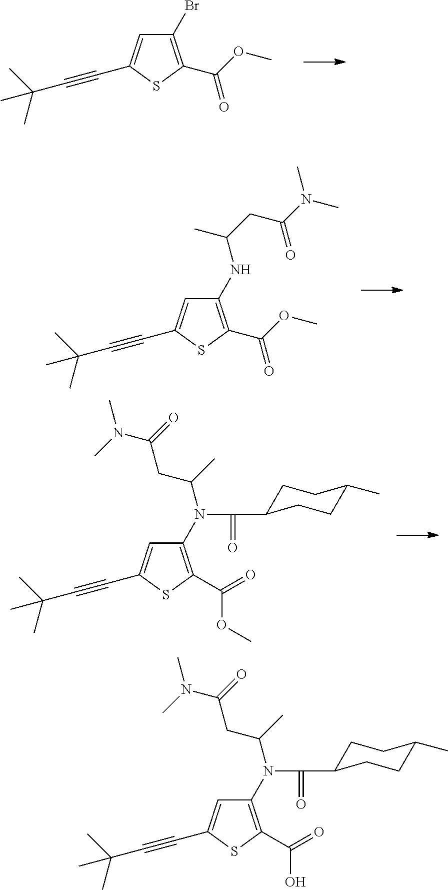

### 3-[[3-(Dimethylamino)-1-methyl-3-oxo-propyl]-(trans-4-methylcyclohexanecarbonyl)amino]-5-(3,3-dimethylbut-1-ynyl)thiophene-2-carboxylic acid

Prepared according to the procedures described in compound 23. LCMS: 459.59 (MH+). 1H NMR (300 MHz, DMSO) δ 13.5 (br, 1H), 7.60 (s, 0.6H), 7.35 (s, 0.4H), 5.01-4.95 (, 0.4H), 4.65-4.49 (m, 0.4H), 3.65 (br, 0.4H), 3.25 (br, 0.6H), 3.00 (s, 1.8H), 3.85 (s, 1.2H), 2.80 (s, 1.2H), 2.75 (s, 1.8H), 2.60-1.75 (m, 2H), 1.60-1.12 (m, 8H), 1.30 (s, 9H), 0.85 (d, 3H), 0.75 (d, 3H), 0.65-0.50 (m, 1H).

### Preparation of Compound 104

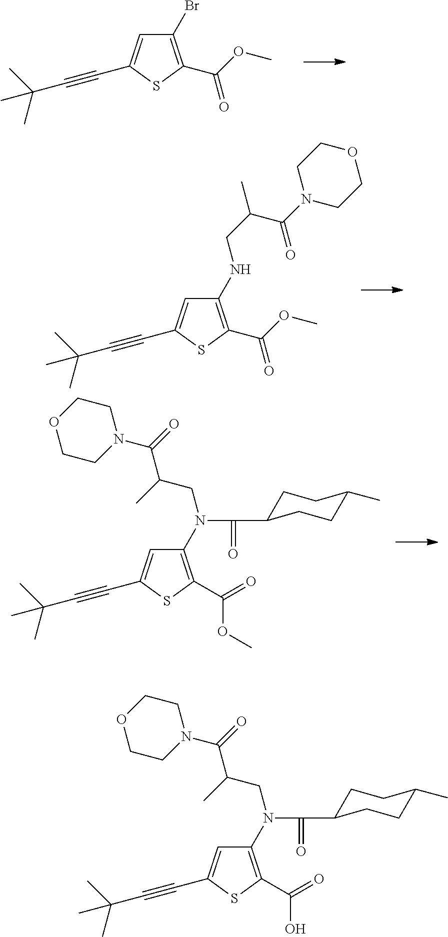

### 5-(3,3-Dimethylbut-1-ynyl)-3-[(trans-4-methylcyclohexanecarbonyl)-(2-methyl-3-morpholino-3-oxo-propyl)amino]thiophene-2-carboxylic acid

Prepared according to the procedures described in compound 23. LCMS: 503.35 (MH+). ¹H NMR (300 MHz, CDCl₃) d 6.94 (s, 0.5H), 6.80 (s, 0.5H), 4.07-3.95 (m, 1H), 3.75-3.33 (m, 100H), 2.15-2.01 (m, 1H), 1.73-1.40 (m, 7H), 1.32 (s, 9H), 1.13-1.02 (m, 3H), 0.80 (d, 3H), 0.80-0.60 (m, 2H).

### Preparation of Compounds 108 & 109

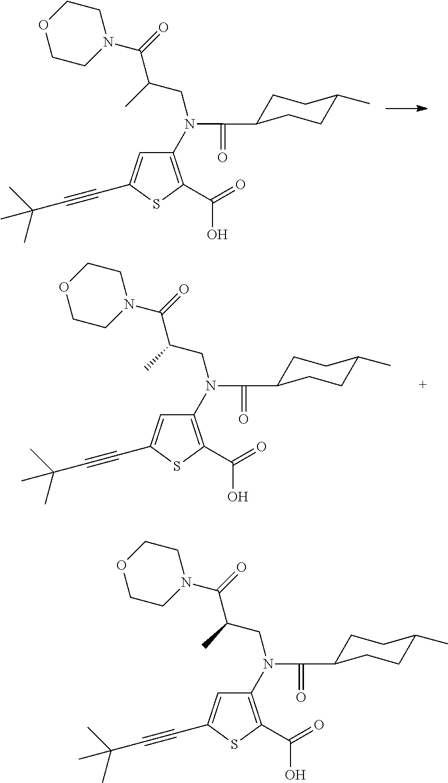

### 5-(3,3-Dimethylbut-1-ynyl)-3-[(trans-4-methylcyclohexanecarbonyl)-[(2S)-2-methyl-3-morpholino-3-oxo-propyl]amino]thiophene-2-carboxylic acid and 5-(3,3-dimethylbut-1-ynyl)-3-[(trans-4-methylcyclohexanecarbonyl)-[(2R)-2-methyl-3-morpholino-3-oxo-propyl]amino]thiophene-2-carboxylic acid

Separated using SFC under following conditions: Column: Whelk-O (10×250); Mobile phase: 15% EtOH, 85% CO2; Flow rate: 10 mL/min; Pressure: 100 bar.

### Preparation of Compound 107

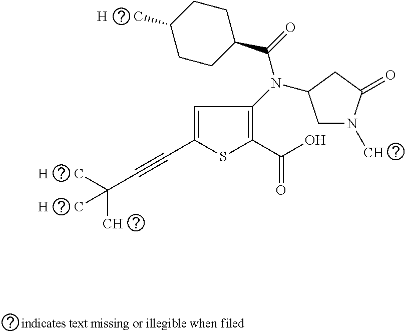

### 5-(3,3-Dimethylbut-1-ynyl)-3-[(trans-4-methylcyclohexanecarbonyl)-(1-methyl-5-oxo-pyrrolidin-3-yl)amino]thiophene-2-carboxylic acid

Prepared according to the procedures described in compound 23. LCMS: 445.25. 1H NMR (300 MHz, CDCl3) d 6.84 (s, 0.5H), 6.80 (s, 0.5H), 5.57-5.34 (m, 0.5H), 4.35-4.23 (m, 0.5H), 3.85-3.61 (m, 1H), 3.33-3.00 (m, 1H), 3.85-3.60 (m, 6H), 2.15-1.85 (m, 1H), 1.70-1.45 (m, 7H), 1.32 (s, 9H), 0.80-0.60 (m, 5H).

### Preparation of Compound 118

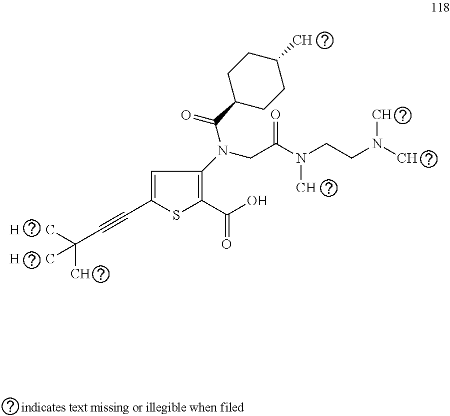

### 3-[[2-[2-Dimethylaminoethyl(methyl)amino]-2-oxo-ethyl]-(trans-4-methylcyclohexanecarbonyl)amino]-5-(3,3-dimethylbut-1-ynyl)thiophene-2-carboxylic acid

Prepared according to the procedures described in compound 6. LCMS: 490.23 (MH+). 1H NMR (300 MHz, CDCl3) d 6.98 (s, 1H), 4.92-4.15 (m, 5H), 3.65-2.73 (br, 100H), 2.35-2.21 (m, 1H), 1.73-1.50 (m, 5H), 1.32 (s, 9H), 1.33-1.22 (m, 2H), 0.85 (d, 3H), 0.80-0.60 (m, 2H).

### Preparation of Compound 119

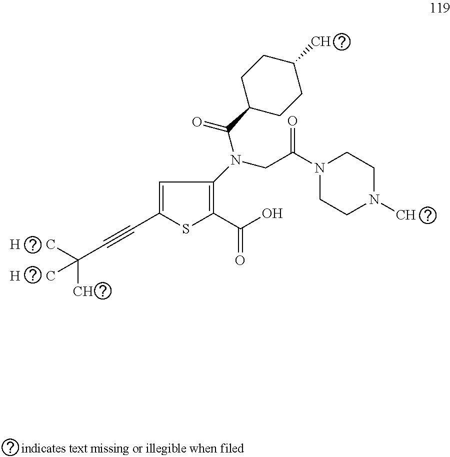

### 5-(3,3-dimethylbut-1-ynyl)-3-[(trans-4-methylcyclohexanecarbonyl)-[2-(4-methylpiperazin-1-yl)-2-oxo-ethyl]amino]thiophene-2-carboxylic acid

Prepared according to the procedures described in compound 6. LCMS: 488.25. H NMR (300 MHz, DMSO) d 7.20 (s, 1H), 3.55-2.71 (m, 8H), 3.45 (s, 3H), 2.75 (s, 2H), 2.15-2.05 (m, 1H), 1.63-1.50 (m, 5H), 1.32 (s, 9H), 1.33-1.22 (m, 2H), 0.75 (d, 3H), 0.70-0.60 (m, 2H).

### Preparation of Compound 120

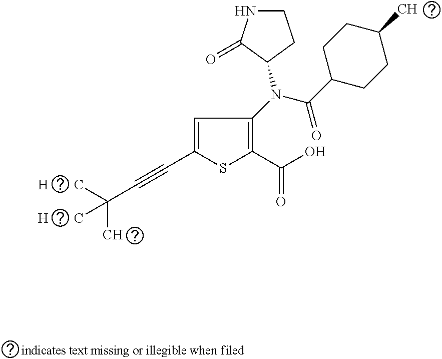

### 5-(3,3-dimethylbut-1-ynyl)-3-[(trans-4-methylcyclohexanecarbonyl)-[(3S)-2-oxopyrrolidin-3-yl]amino]thiophene-2-carboxylic acid

Prepared according to the procedures described in compound 23. LCMS: 431.24 (MH+). ¹H NMR (300 MHz, CDCl₃) δ 7.04 (s, 1H), 6.59 (d, J=5.5 Hz, 1H), 4.67 (dd, J=14.7, 9.8 Hz, 1H), 3.97-3.78 (m, 1H), 3.68 (t, J=8.7 Hz, 1H), 2.90-2.71 (m, 1H), 2.23-1.67 (m, 7H), 1.61-1.38 (m, 2H), 1.32 (s, 9H), 1.03-0.82 (m, 5H).

### Preparation of Compound 100

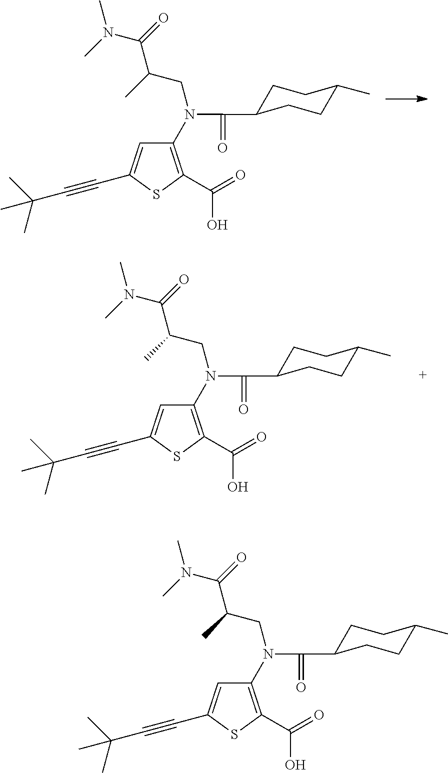

### 3-[[(1R)-3-(dimethylamino)-1-methyl-3-oxo-propyl]-(trans-4-methylcyclohexanecarbonyl)amino]-5-(3,3-dimethylbut-1-ynyl)thiophene-2-carboxylic acid

Isolated using SFC under following conditions: Column: Whelk-O (10×250); Mobile phase: 15% EtOH, 85% CO2; Flow rate: 10 mL/min; Pressure: 100 bar.

### Preparation of Compound 55

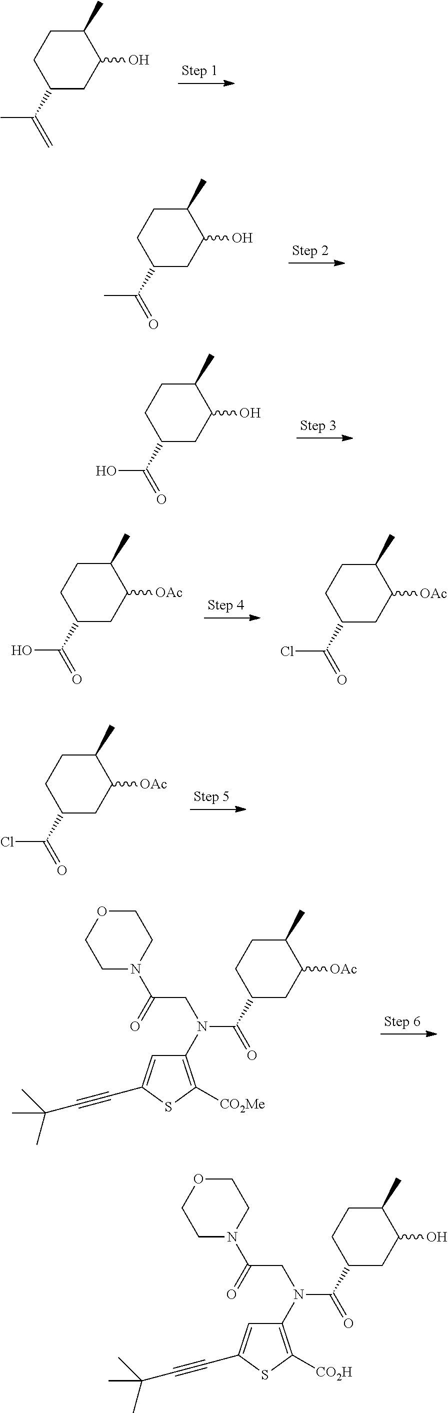

### Step 1

### 1-((1R,4R)-3-hydroxy-4-methylcyclohexyl)ethanone

Ozone was bubbled through a solution of (2R,5R)-5-isopropenyl-2-methyl-cyclohexanol (5 g, 32.4 mmol, commercial 25:75% epimeric mixture in favor of the (1R)-alcohol) in DCM (140 mL) and MeOH (3 mL) at −78° C. until the solution turned blue (30 min through a fritted diffuser). The excess of ozone was flushed off with oxygen (5 min). Dimethylsulfide (10 mL, 137 mmol) was added at the same temperature and the reaction mixture was slowly warmed to RT and stirred overnight. The reaction mixture was concentrated and the resulting oil purified by silica gel chromatography (120 g ISCO column, eluted with a gradient of 15% to 40% ether in hexane) to afford 1-[(1R,4R)-3-hydroxy-4-methyl-cyclohexyl]ethanone (4.23 g, 84%, colorless oil). The product was taken into the next step without further analysis.

### Step 2

### (1R,4R)-3-hydroxy-4-methylcyclohexanecarboxylic acid

To an ice-cold solution of NaOH (14.1 g, 352 mmol) in water (120 mL) and 1,4-dioxane (90 mL) was added bromine (14.3 g, 89.4 mmol). To the resultant yellow solution was added dropwise, a solution of 1-((1R,4R)-3-hydroxy-4-methylcyclohexyl)ethanone (4.23 g, 27.1 mmol) in dioxane (200 mL) and water (54 mL). The resulting solution was stirred at 10-15° C. for 3 h. The excess of NaOBr was decomposed by adding a solution of sodium sulfite (3.3 g in 30 mL of water), acidified with 10% HCl and extracted with DCM. The combined organic extracts was washed with brine, dried and concentrated to afford (1R,4R)-3-hydroxy-4-methylcyclohexanecarboxylic acid (4.17 g, 90%).

### Step 3

### (1R,4R)-3-acetoxy-4-methylcyclohexanecarboxylic acid

To a solution of (1R,4R)-3-hydroxy-4-methylcyclohexanecarboxylic acid (4.17 g, 26.4 mmol) in DCM (120 mL) was added pyridine (12.5 g, 158 mmol) followed by acetic anhydride (10.8 g, 105 mmol). The reaction mixture was stirred for 15 h at RT. The solvents were removed in vacuo and 3N aq. HCl (25 mL) was added. The reaction mixture was stirred for 30 min and then a sat. aq. NaHCO₃ (500 mL) was slowly added until mixture reached pH 9-10. This solution was then extracted with EtOAc (2×150 mL). The aqueous phase was acidified with 10% aq. HCl and extracted with EtOAc (3×100 mL). The combined organic layers were dried over Na₂SO₄, and concentrated to afford (1R,4R)-3-(ethanoyloxy)-4-methylcyclohexanecarboxylic acid (2.37 g, 45%).

### Step 4

### (2R,5R)-5-(chlorocarbonyl)-2-methylcyclohexyl acetate

To a solution of (1R,4R)-3-(ethanoyloxy)-4-methylcyclohexanecarboxylic acid (666 mg, 3.33 mmol) in DCM (13 mL) was added DMF (2 drops) and then oxalyl chloride (464 mg, 3.66 mmol) carefully with ice-bath cooling. The ice-bath was removed after 5 min. After a further 4 h, the reaction mixture was concentrated and the residue used directly in the next step.

### Step 5

### Methyl 5-(3,3-dimethylbut-1-ynyl)-3-((1R,4R)-3-(ethanoyloxy)-4-methyl-N-(2-morpholino-2-oxoethyl)cyclohexanecarboxamido)thiophene-2-carboxylate

A round-bottomed flask was charged with methyl 5-(3,3-dimethylbut-1-ynyl)-3-[(2-morpholino-2-oxo-ethyl)amino]thiophene-2-carboxylate (606 mg, 1.66 mmol) in DCE (24 mL), (2R,5R)-5-(chlorocarbonyl)-2-methylcyclohexyl ethanoate (727 mg, 3.33 mmol) followed by pyridine (1.97 g, 24.9 mmol) and DMAP (10 mg, 0.083 mmol). The solution was refluxed overnight. Water and brine were added and the mixture extracted twice with EtOAc. The combined organic layers were washed twice with 1N aq. HCl, then 1N aq. NaOH, dried over Na₂SO₄, filtered and concentrated. The residue was purified by column chromatography (100-200 mesh silica gel) using 50% EtOAc in hexane as eluent to afford methyl 5-(3,3-dimethylbut-1-ynyl)-3-((1R,4R)-3-(ethanoyloxy)-4-methyl-N-(2-morpholino-2-oxoethyl)cyclohexanecarboxamido)thiophene-2-carboxylate (347 mg, 38%). TLC system: 50% EtOAc in hexane. Rf: 0.27. MS: m/z (obs.) 547.31 [M+H]⁺.

### Step 6

### Compound 55

Methyl 5-(3,3-dimethylbut-1-ynyl)-3-((1R,4R)-3-(ethanoyloxy)-4-methyl-N-(2-morpholino-2-oxoethyl)cyclohexanecarboxamido)thiophene-2-carboxylate (347 mg, 0.635 mmol) was dissolved in a mixture of MeOH (3 mL) and 2N aq. NaOH (3 mL, 6.00 mmol) and stirred overnight at RT. Brine (6 mL), 1N aq. HCl (7 mL) and water (10 mL) were added. The mixture was extracted with EtOAc, dried over Na₂SO₄, filtered and concentrated. The residue was purified by Gilson prep HPLC [gradient: 40% solvent B to 70% solvent B. B=MeCN (containing 0.1% TFA); A=water (containing 0.1% TFA+1% MeCN)] to afford 55 (198 mg, 63%, white solid).

### Preparation of Compound 71

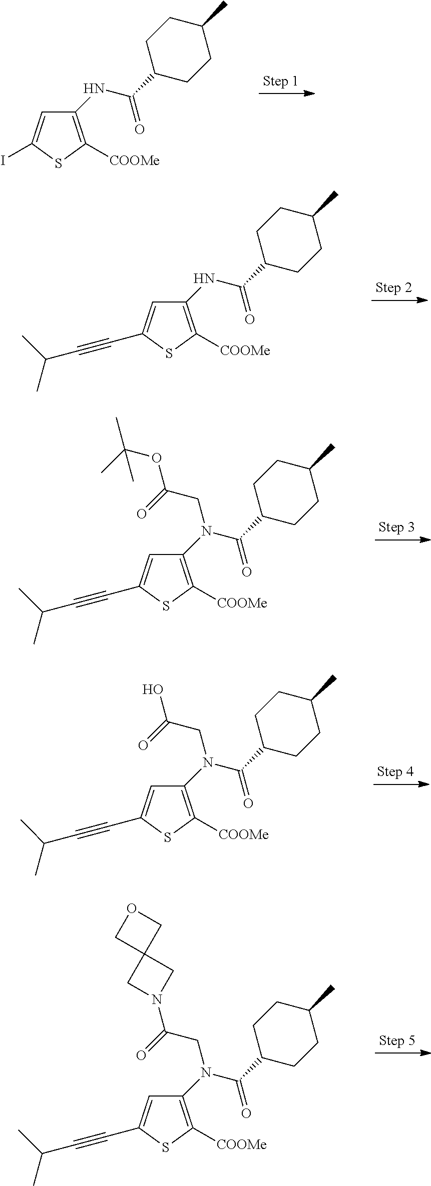

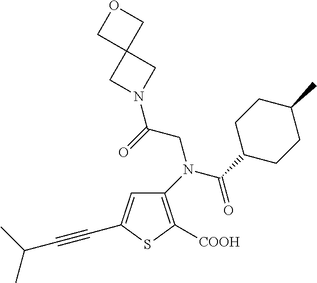

### Step 1

### Methyl 5-(3-methylbut-1-ynyl)-3-((1R,4R)-4-methylcyclohexanecarboxamido)thiophene-2-carboxylate

Charged a 100 mL, 2-necked round bottomed flask with methyl 5-iodo-3-[(trans-4-methylcyclohexanecarbonyl)amino]thiophene-2-carboxylate (2.85 g, 7.01 mmol), DMF (28 mL) and triethylamine (2.13 g, 2.93 mL, 21.0 mmol) under nitrogen. In rapid succession, added 3-methylbut-1-yne (4 mL), copper(I) iodide (200 mg, 1.05 mmol) and PdCl₂(PPh₃)₂ (98 mg, 0.14 mmol). After one day, EtOAc and water were added to the reaction mixture. The mixture was filtered through Celite and then the layers were separated. The aqueous layer was re-extracted with EtOAc and the combined organics were washed with brine, dried over Na₂SO₄ and concentrated. Purification by column chromatography (100-200 mesh silica gel) using 5% EtOAc in hexane as eluent to afford methyl 5-(3-methylbut-1-ynyl)-3-((1R,4R)-4-methylcyclohexanecarboxamido)thiophene-2-carboxylate (1.78 g, 73%, off-white solid). MS: m/z (obs.) 348 [M+H]⁺.

### Step 2

### Methyl 3-((1R,4R)—N-(2-tert-butoxy-2-oxoethyl)-4-methylcyclohexanecarboxamido)-5-(3-methylbut-1-ynyl)thiophene-2-carboxylate

A solution of methyl 5-(3-methylbut-1-ynyl)-3-((1R,4R)-4-methylcyclohexanecarboxamido)thiophene-2-carboxylate (1.35 g, 3.89 mmol) and THF (15 mL) was cooled in an ice-bath under nitrogen. LiHMDS (5.05 mL of 1 M solution in THF, 5.05 mmol) was added, stirring continued at 0° C. for 10 min and then the cold bath was removed. After 25 min, tert-butyl 2-bromoacetate (909 mg, 4.66 mmol) was added and the mixture heated at 60° C. overnight. Water and EtOAc were added. The layers were separated and the aqueous layer was re-extracted with EtOAc. The combined organics were washed with brine and then water, dried over Na₂SO₄ and concentrated. Purification by column chromatography (100-200 mesh silica gel) using 5% to 20% EA in pet ether to afford methyl 3-((1R,4R)—N-(2-tert-butoxy-2-oxoethyl)-4-methylcyclohexanecarboxamido)-5-(3-methylbut-1-ynyl)thiophene-2-carboxylate (1.13 g, 63%, colourless oil). MS: m/z (obs.) 462 [M+H]⁺.

### Step 3

### 2-((1R,4R)—N-(2-(methoxycarbonyl)-5-(3-methylbut-1-ynyl)thiophen-3-yl)-4-methylcyclohexanecarboxamido)ethanoic acid

To a solution of methyl 3-((1R,4R)—N-(2-tert-butoxy-2-oxoethyl)-4-methylcyclohexanecarboxamido)-5-(3-methylbut-1-ynyl)thiophene-2-carboxylate (1.13 g, 2.45 mmol) in DCM (15 mL) was added TFA (3 mL, 38.9 mmol) at RT. After 3 h, the reaction was concentrated in vacuo at <30° C. The residue was filtered through a pad of silica with EtOAc and then concentrated to give 2-((1R,4R)—N-(2-(methoxycarbonyl)-5-(3-methylbut-1-ynyl)thiophen-3-yl)-4-methylcyclohexanecarboxamido)ethanoic acid (812 mg, 82%, white solid). MS: m/z (obs.) 406 [M+H]⁺.

### Step 4

### Methyl 3-((1R,4R)-4-methyl-N-(2-oxo-2-(2-oxa-6-azaspiro[3.3]heptan-6-yl)ethyl)cyclohexanecarboxamido)-5-(3-methylbut-1-ynyl)thiophene-2-carboxylate

To a solution of 2-((1R,4R)—N-(2-(methoxycarbonyl)-5-(3-methylbut-1-ynyl)thiophen-3-yl)-4-methylcyclohexanecarboxamido)ethanoic acid (148 mg, 0.366 mmol) in DCM (6 mL) containing DIPEA (156 mg, 1.21 mmol) was added 3-(ethyliminomethyleneamino)-N,N-dimethyl-propan-1-amine (68 mg, 0.439 mmol) then 6-oxa-2-azaspiro[3.3]heptane (204 mg, 0.731 mmol) at RT under nitrogen. After stirring overnight at RT, the reaction was concentrated in vacuo. EtOAc and water were added and the aqueous layer was separated and re-extracted with EtOAc. The combined organics were dried over Na₂SO₄ and concentrated. Crude methyl 3-((1R,4R)-4-methyl-N-(2-oxo-2-(2-oxa-6-azaspiro[3.3]heptan-6-yl)ethyl)cyclohexanecarboxamido)-5-(3-methylbut-1-ynyl)thiophene-2-carboxylate was taken into the following step without further purification. MS: m/z (obs.) 487 [M+H]⁺.

### Step 5

### Compound 71

To a solution of methyl 3-((1R,4R)-4-methyl-N-(2-oxo-2-(2-oxa-6-azaspiro[3.3]heptan-6-yl)ethyl)cyclohexanecarboxamido)-5-(3-methylbut-1-ynyl)thiophene-2-carboxylate (86 mg, 0.177 mmol) in MeOH (4 mL) was added 2N aq. NaOH (3.5 mL, 7.00 mmol) at RT and the mixture stirred vigorously. After 30 min, brine (6 mL) and 2N aq. HCl (8 mL) were added and the mixture extracted twice with EtOAc. The combined organics were dried over Na₂SO₄ and concentrated. The residue was purified by Gilson preparatory HPLC with aq. TFA/acetonitrile mixtures to afford 71 (53 mg, 60%, white solid).

### Preparation of Compound 62

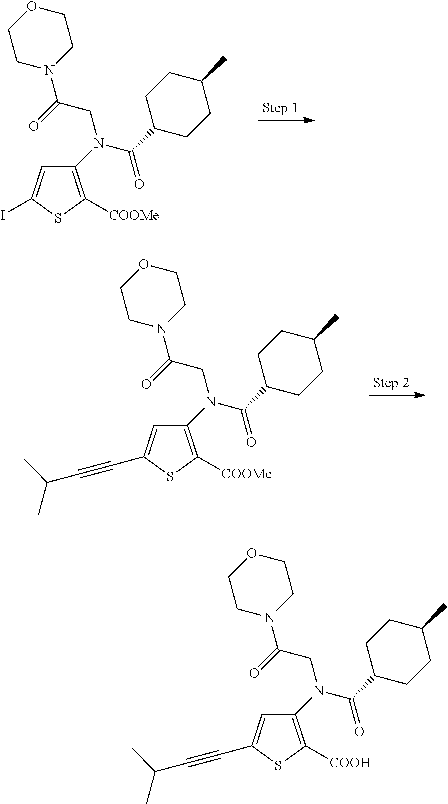

### Step 1

### Methyl 3-((1R,4R)-4-methyl-N-(2-morpholino-2-oxoethyl)cyclohexanecarboxamido)-5-(3-methylbut-1-ynyl)thiophene-2-carboxylate

Charged a 100 mL 3-necked flask with starting iodide (856 mg, 1.60 mmol), DMF (8.6 mL), triethylamine (486 mg, 670 μL, 4.81 mmol) and 3-methylbut-1-yne (142 mg, 2.08 mmol). The mixture was cooled in an ice-bath and after 10 min, in rapid succession were added iodocopper (45.8 mg, 0.240 mmol) then PdCl₂(PPh₃)₂ (22.5 mg, 0.032 mmol) and the cold bath removed. After 5 h, added a further 2.5 mL of 3-methylbut-1-yne. Stirring was continued for 1 day and then EtOAc and water were added to the mixture. Filtered through Celite and then separated. The aqueous layer was re-extracted with EtOAc and the combined organics were washed with brine, dried over Na₂SO₄ and concentrated. Purification by column chromatography (100-200 mesh silica gel) using EtOAc as eluent to afford methyl 3-((1R,4R)-4-methyl-N-(2-morpholino-2-oxoethyl)cyclohexanecarboxamido)-5-(3-methylbut-1-ynyl)thiophene-2-carboxylate (448 mg, 59%, white solid). MS: m/z (obs.) 475.45 [M+H]⁺.

### Step 2

### Compound 62

Methyl 3-((1R,4R)-4-methyl-N-(2-morpholino-2-oxoethyl)cyclohexanecarboxamido)-5-(3-methylbut-1-ynyl)thiophene-2-carboxylate (248 mg, 0.523 mmol) was dissolved in a mixture of MeOH (3 mL) and 2N aq. NaOH (3 mL, 6.00 mmol) and the mixture was stirred vigorously. After 90 min, brine (6 mL), 1N aq. HCl (7 mL), and water (10 mL) were added. The mixture was extracted with EtOAc and the organic layer was dried over Na₂SO₄, filtered and concentrated. The residue was purified by Gilson preparatory HPLC (Gradient: 40% solvent B to 70% solvent B. B=MeCN (containing 0.1% TFA); A=water (containing 0.1% TFA+1% MeCN) to afford 62 (78.7 mg, 32%, white solid).

### Preparation of Compound 67

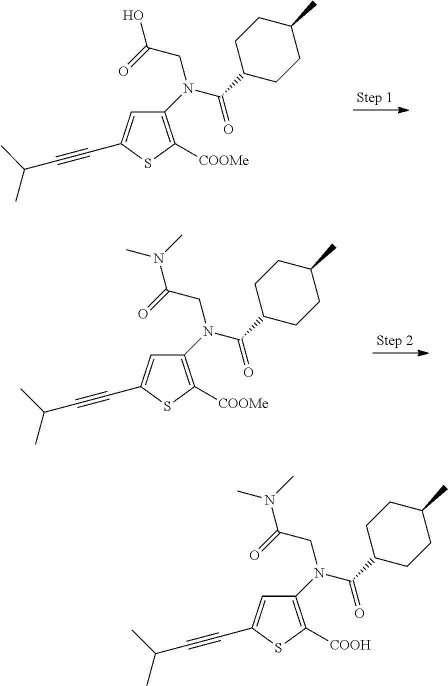

### Step 1

### Compound 67, methyl ester

To a solution of starting acid (148 mg, 0.366 mmol) in DCM (6 mL) containing DIPEA (61.4 mg, 0.475 mmol) was added 3-(ethyliminomethyleneamino)-N,N-dimethyl-propan-1-amine (EDC, 68.1 mg, 0.439 mmol) and after 10 min dimethylamine in water (123 mg, 138 μL of 40% w/w solution, 1.10 mmol) at RT under nitrogen. After 1 h, water and brine were added and the layers separated. The aqueous layer was re-extracted with DCM and the combined organics were dried over Na₂SO₄, filtered and concentrated. This residue was taken directly on in the next step without further purification. MS: m/z (obs.) 433.42 [M+H]⁺

### Step 2

### Compound 67

To a solution of starting ester (158 mg, 0.366 mmol) in MeOH (3.5 mL) was added 2N aq. NaOH (3.5 mL, 7.00 mmol) at RT with vigorous stirring. After 1 h, brine (6 mL) and then 2N aq. HCl (8 mL) were added and the mixture extracted twice with EtOAc. The combined organics were dried over Na₂SO₄, filtered and concentrated. The residue was purified by Gilson preparatory HPLC (Gradient: 10% solvent B to 90% solvent B. B=MeCN (containing 0.1% TFA); A=water (containing 0.1% TFA+1% MeCN) to afford 67 (9.4 mg, 6%, white solid).

### Preparation of Compound 69

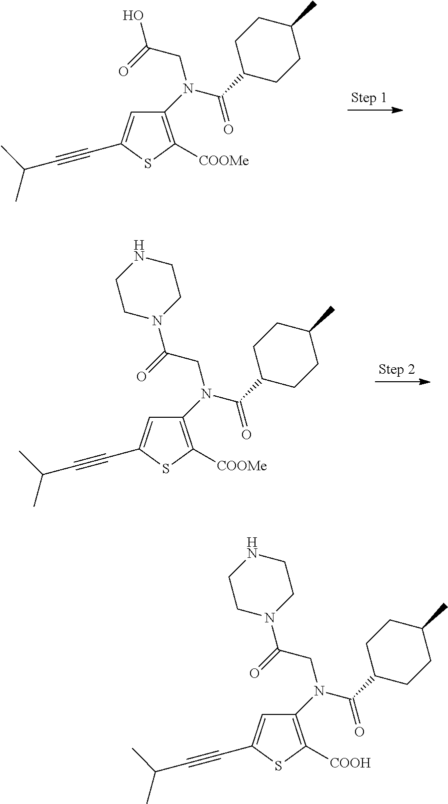

### Step 1

### Compound 69, methyl ester

To a solution of starting acid (148 mg, 0.366 mmol) in DCM (6 mL) containing DIPEA (61.4 mg, 0.475 mmol) was added 3-(ethyliminomethyleneamino)-N,N-dimethyl-propan-1-amine (EDC, 68.1 mg, 0.439 mmol) followed after 5 min by piperazine (63 mg, 0.731 mmol) at RT under nitrogen. After 6 h, the mixture was concentrated in vacuo. EtOAc and brine were added. The aqueous layer was separated and re-extracted with EtOAc. The combined organics were dried over Na₂SO₄, filtered and concentrated. This residue was taken directly on in the next step without further purification. MS: m/z (obs.) 474.03 [M+H]⁺.

### Step 2

### Compound 69

To a solution of Methyl 3-((1R,4R)-4-methyl-N-(2-oxo-2-(piperazin-1-yl)ethyl)cyclohexanecarboxamido)-5-(3-methylbut-1-ynyl)thiophene-2-carboxylate (173 mg, 0.366 mmol) in MeOH (3 mL) was added 2N aq. NaOH (3 mL, 6.00 mmol) at RT with vigorous stirring. After 30 min, the mixture was concentrated in vacuo at <33° C. The residue was purified by Gilson preparatory HPLC (Gradient: 10% solvent B to 90% solvent B. B=MeCN (containing 0.1% TFA); A=water (containing 0.1% TFA+1% MeCN) to afford 69 (16.3 mg, 10%, white solid).

### Preparation of Compound 70

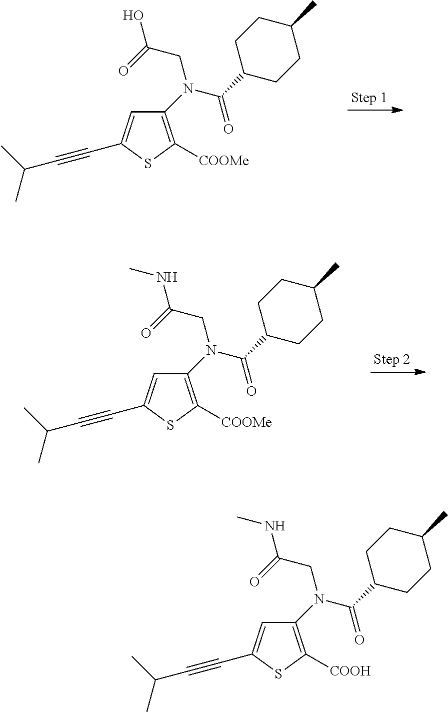

### Step 1

### Compound 70, methyl ester

To a solution of starting acid (182 mg, 0.449 mmol) in DCM (8 mL) containing DIPEA (174 mg, 235 μL, 1.35 mmol) was added DMAP (2.7 mg, 22.4 μmol), 3-(ethyliminomethyleneamino)-N,N-dimethyl-propan-1-amine (EDC, 139 mg, 0.898 mmol) then 2N methylamine in THF (898 μL, 1.80 mmol) at RT under nitrogen. After stirring overnight, water and brine were added and the mixture extracted with DCM. The organics were dried (Na₂SO₄), filtered and concentrated. The residue was filtered through a pad of silica with EtOAc and the concentrated filtrate was taken directly on in the next step. MS: m/z (obs.) 419.39 [M+H]⁺.

### Step 2

### Compound 70

To a solution of methyl 3-((1R,4R)-4-methyl-N-(2-(methylamino)-2-oxoethyl)cyclohexanecarboxamido)-5-(3-methylbut-1-ynyl)thiophene-2-carboxylate (84 mg, 0.201 mmol) in MeOH (4 mL) was added 2N aq. NaOH (3.5 mL, 7.00 mmol) and the mixture was stirred vigorously. After 30 min, brine (6 mL) and 2N aq. HCl (8 mL) were added and the mixture extracted twice with EtOAc. The combined organics were dried over Na₂SO₄, filtered and concentrated. The residue was purified by Gilson preparatory HPLC (Gradient: 15% solvent B to 90% solvent B. B=MeCN (containing 0.1% TFA); A=water (containing 0.1% TFA+1% MeCN) to afford 70 (58.5 mg, 71%, white solid)

### Preparation of Compound 82

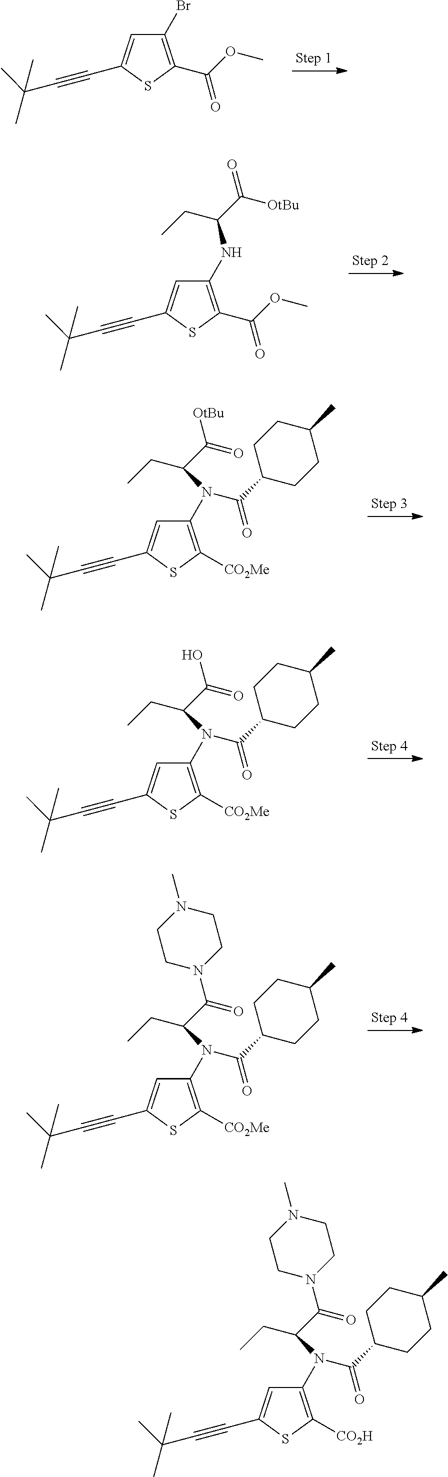

### Step 1

### (S)-Methyl 3-((1-(tert-butoxy)-1-oxobutan-2-yl)amino)-5-(3,3-dimethylbut-1-yn-1-yl)thiophene-2-carboxylate

A mixture of methyl 3-bromo-5-(3,3-dimethylbut-1-ynyl)thiophene-2-carboxylate (1 g, 3.3 mmol), rac-BINAP (413 mg, 0.66 mmol), tert-butyl (2S)-2-aminobutanoate hydrochloride (780 mg, 3.98 mmol), Pd₂(dba)₃ (304 mg, 0.33 mmol) and cesium carbonate (3.25 g, 10 mmol) was degassed and filled with N₂, and 1,4-dioxane (15 mL) was added. The mixture was bubbled N₂ for 30 min and stirred at 90° C. for 16 h. Reaction progress was monitored by LC-MS. On completion the reaction mixture was cooled to RT, diluted with EtOAc (20 mL) and filtered though a layer of celite. The filtrate was concentrated in vacuum. The residue was purified by silica gel column chromatography, eluting with 0-10% EtOAc in hexane to afford (S)-methyl 3-((1-(tert-butoxy)-1-oxobutan-2-yl)amino)-5-(3,3-dimethylbut-1-yn-1-yl)thiophene-2-carboxylate (1.1 g, 83%, colorless oil). MS: m/z (obs.) 378 [M+1]⁺.

### Step 2

### Methyl 3-[[(1S)-1-tert-butoxycarbonylpropyl]-(trans-4-methylcyclohexanecarbonyl)amino]-5-(3,3-dimethylbut-1-ynyl)thiophene-2-carboxylate

To a solution of methyl 3-[[(1S)-1-tert-butoxycarbonylpropyl]amino]-5-(3,3-dimethylbut-1-ynyl)thiophene-2-carboxylate (1.05 g, 2.77 mmol) in DCE (10 mL) was added pyridine (2.2 g, 2.2 mL, 27.7 mmol), DMAP (68 mg, 0.55 mmol) and trans-4-methylcyclohexanecarbonyl chloride (2.22 g, 13.8 mmol). The mixture was stirred for 16 h at 90° C. On completion the reaction mixture was cooled to RT, diluted with EtOAc (30 mL), washed with NaHCO₃ solution (2×20 mL), dried over Na₂SO₄ and concentrated in vacuum. The residue was purified by silica gel chromatography, eluting with 0-10% EtOAc in hexane to afford methyl 3-[[(1S)-1-tert-butoxycarbonylpropyl]-(trans-4-methylcyclohexanecarbonyl)amino]-5-(3,3-dimethylbut-1-ynyl)thiophene-2-carboxylate (1.0 g, 71.7%, white solid). MS: m/z (obs.) 526 [M+1]⁺.

### Step 3

### (S)-2-((1r,4S)—N-(5-(3,3-dimethylbut-1-yn-1-yl)-2-(methoxycarbonyl)thiophen-3-yl)-4-methylcyclohexanecarboxamido)butanoic acid

A solution of methyl 3-[[(1S)-1-tert-butoxycarbonylpropyl]-(trans-4-methylcyclohexanecarbonyl)amino]-5-(3,3-dimethylbut-1-ynyl)thiophene-2-carboxylate (900 mg, 1.8 mmol) in TFA (5 mL, 64.9 mmol) was stirred for 1.5 h. The reaction mixture was concentrated in vacuum. The residue was diluted with EtOAc (30 mL), washed with H₂O (2×15 mL) and brine (20 mL), dried over Na₂SO₄ and concentrated to afforded (S)-2-((1r,4S)—N-(5-(3,3-dimethylbut-1-yn-1-yl)-2-(methoxycarbonyl)thiophen-3-yl)-4-methylcyclohexanecarboxamido)butanoic acid (800 mg, 99%, white solid). MS: m/z (obs.) 448 [M+1]⁺.

### Step 4

### Compound 82, methyl ester

To a solution of (2S)-2-[[5-(3,3-dimethylbut-1-ynyl)-2-methoxycarbonyl-3-thienyl]-(trans-4-methylcyclohexanecarbonyl)amino]butanoic acid (90 mg, 0.2 mmol)) in DMF (1 mL) was added DIEA (0.14 mL, 0.8 mmol), HBTU (114 mg, 0.3 mmol) and 1-methyl piperzine (60 mg, 0.6 mmol). The reaction mixture was stirred overnight and then diluted with EtOAc (15 mL), washed with H₂O (10 mL), brine (10 mL), dried over Na₂SO₄ and concentrated in vacuum. The residue was purified by silica gel column chromatography eluting with 0-50% EtOAc in hexane to afford compound 82, methyl ester (62 mg, 58% yield). MS: m/z (obs.) 530 [M+1]⁺.

### Step 5

### Compound 82

To a solution of methyl ester in MeOH (1.5 mL) was added NaOH (0.75 mL, 1M, 0.75 mmol). The solution was stirred overnight and then neutralized with HCl to PH=3.0. The mixture was concentrated in vacuum. The residue was purified by column chromatography eluting with 0-10% MeOH in DCM to afford 82 (48 mg, yield 72%, white solid). MS: m/z (obs.) 516 [M+1]⁺; ¹H NMR (300 MHz, CDCl3) δ 6.82 (s, 1H), 5.32 (s, 1H), 2.89 (d, J=5.4 Hz, 4H), 2.54-2.32 (m, 16H), 1.30 (d, J=30.3 Hz, 11H), 1.03-0.56 (m, 9H).

### Preparation of Compound 65

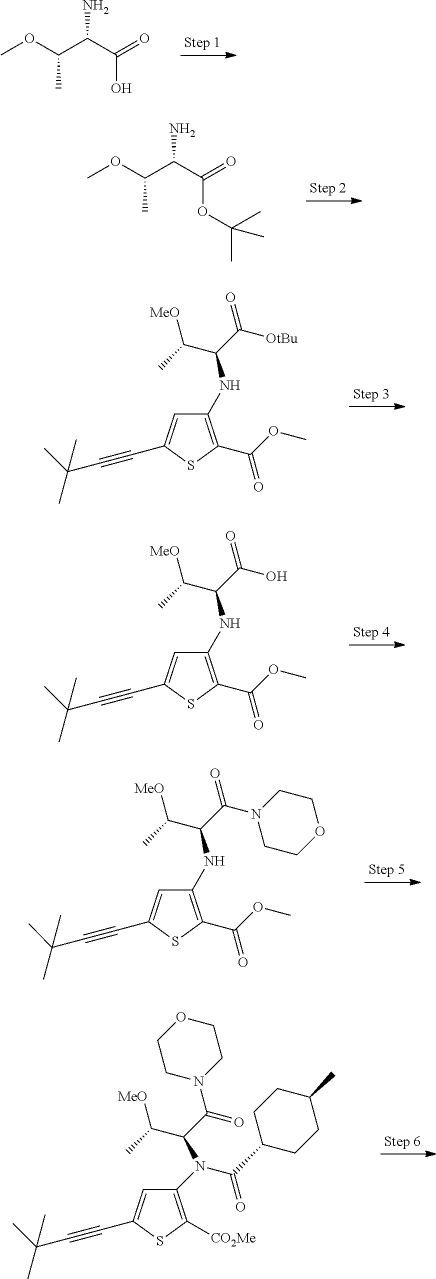

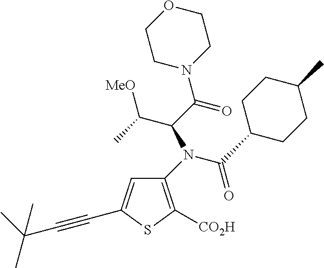 [SMILES: CO[C@@H](C)[C@@H](C(=O)N1CCOCC1)N(c1cc(C#CC(C)(C)C)sc1C(=O)O)C(=O)[C@H]1CC[C@H](C)CC1]

### Step 1

### (2S,3S)-tert-butyl 2-amino-3-methoxybutanoate

To a solution of (2S,3S)-2-amino-3-methoxy-butanoic acid (500 mg, 3.7 mmol) in tert-BuOAc (100 mL) was added perchloric acid (377 mg, 0.23 mL, 3.75 mmol) and stirred for 3 days at RT. The reaction mixture was poured into sat. NaHCO₃ solution (15 mL), extracted with EtOAc (2×20 mL), dried over Na₂SO₄ and concentrated to afford (2S,3S)-tert-butyl 2-amino-3-methoxybutanoate (530 mg, 72%, white solid). ¹H NMR (300 MHz, CDCl₃) δ 3.68 (dd, J=13.8, 5.5 Hz, 1H), 3.64-3.47 (m, 1H), 3.27 (d, J=11.1 Hz, 3H), 1.96 (d, J=7.0 Hz, 2H), 1.41 (s, 9H), 1.08 (t, J=7.6 Hz, 3H).

### Step 2

### Methyl 3-(((2S,3S)-1-(tert-butoxy)-3-methoxy-1-oxobutan-2-yl)amino)-5-(3,3-dimethylbut-1-yn-1-yl)thiophene-2-carboxylate

To a solution of methyl 3-bromo-5-(3,3-dimethylbut-1-ynyl)thiophene-2-carboxylate (649 mg, 2.2 mmol) in Dioxane (10 mL) was added tert-butyl (2S,3S)-2-amino-3-methoxy-butanoate (530 mg, 2.8 mmol), rac-BINAP (268 mg, 0.43 mmol), Pd2(dba)₃ (197 mg, 0.21 mmol) and dicesium carbonate (2.1 g, 6.5 mmol) under N2. The reaction mixture was bubbled N2 for 30 min and heated to 90° C. for 18 h. The reaction was cooled down to RT, diluted with EtOAc (20 mL) and filtered though a layer of celite. The filtrate was concentrated. The residue was purified by silica gel chromatography eluting with 0-10% EtOAc in hexane to afford methyl 3-(((2S,3S)-1-(tert-butoxy)-3-methoxy-1-oxobutan-2-yl)amino)-5-(3,3-dimethylbut-1-yn-1-yl)thiophene-2-carboxylate (590 mg, 1.4 mmol, 67%, yellow oil). MS: m/z (obs.) 409.88 [M+1]⁺

### Step 3

A solution of methyl 3-(((2S,3S)-1-(tert-butoxy)-3-methoxy-1-oxobutan-2-yl)amino)-5-(3,3-dimethylbut-1-yn-1-yl)thiophene-2-carboxylate (520 mg, 1.270 mmol) in TFA (2.0 mL, 25.96 mmol) was stirred for 2 h at RT. The mixture was concentrated, diluted with EtOAc (20 mL), washed with H₂O (2×15 mL) and brine (15 mL), dried over Na2SO4, and concentrated. The residue is carried to next step. MS: m/z (obs.) 353.89 [M+1]⁺

### Step 4

### Methyl 5-(3,3-dimethylbut-1-yn-1-yl)-3-(((2S,3S)-3-methoxy-1-morpholino-1-oxobutan-2-yl)amino)thiophene-2-carboxylate

To a solution of (2S,3S)-2-((5-(3,3-dimethylbut-1-yn-1-yl)-2-(methoxycarbonyl)thiophen-3-yl)amino)-3-methoxybutanoic acid (90 mg, 0.25 mmol) in DMF (1 mL) was added HBTU (145 mg, 0.38 mmol), DIEA (99 mg, 0.14 mL, 0.76 mmol) and morpholine (166 mg, 0.16 mL 0.76 mmol). The reaction mixture was stirred for 2 h at RT and diluted with EtOAc (15 mL), washed with H₂O (15 mL) and brine (15 mL), concentrated. The residue was purified by silica gel chromatography eluting with 0-50% EtOAc in hexane to afford methyl 5-(3,3-dimethylbut-1-yn-1-yl)-3-(((2S,3S)-3-methoxy-1-morpholino-1-oxobutan-2-yl)amino)thiophene-2-carboxylate (88 mg, 82%). MS: m/z (obs.) 422.9 [M+1]⁺

### Step 5

### Compound 65, methyl ester

To a solution of 4 methyl 5-(3,3-dimethylbut-1-yn-1-yl)-3-(((2S,3S)-3-methoxy-1-morpholino-1-oxobutan-2-yl)amino)thiophene-2-carboxylate (88 mg, 0.21 mmol) in DCE (2 mL) was added pyridine (165 mg, 0.17 mL, 2.1 mmol) and DMAP (3 mg, 0.02 mmol) and 4-methylcyclohexanecarbonyl chloride (167 mg, 1.0 mmol). The resulting reaction mixture was stirred for 15 h at 90° C. Reaction progress was monitored by LC-MS. On completion the reaction mixture was cooled to RT, diluted with EtOAc (20 mL), washed with H₂O (15 mL), brine (15 mL) dried over Na₂SO₄, and concentrated. The residue was purified by silica gel chromatography, eluting with 0-50% EtOAc in hexane to afford desired compound 65, methyl ester (50 mg, 72%). MS: m/z (obs.) 546.9 [M+1]⁺

### Step 6

### Compound 65

Starting ester (50 mg, 0.09 mmol) was dissolved in MeOH (3 mL) and NaOH (0.46 mL of 1.0 M, 0.46 mmol) was added. The reaction mixture was stirred overnight at RT, then neutralized to PH=3 with 1N HCl and the solution was concentrated. The residue was purified by silica gel chromatography, eluting with 0-4% MeOH in DCM to afford 65 (37 mg, 77%). MS: m/z (obs.) 532.9 [M+1]⁺; ¹H NMR (300 MHz, CDCl₃) δ 7.95 6.80 (d, J=3.1 Hz, 1H), 5.57-5.23 (m, 1H), 4.01-3.55 (m, 9H), 3.54-3.23 (m, 3H), 3.14 (m, 4H), 2.04 (m, 1H), 1.67 1.58 (m, 4H), 1.45-1.19 (m, 12H), 1.11-0.96 (m, 1H), 0.91-0.47 (m, 4H).

### Preparation of Compound 66

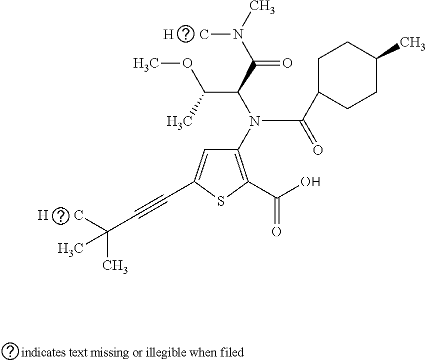

### 5-(3,3-Dimethylbut-1-ynyl)-3-[[(1S,2S)-1-(dimethylcarbamoyl)-2-methoxy-propyl]-(trans-4-methylcyclohexanecarbonyl)amino]thiophene-2-carboxylic acid

Compound 66 was made by the same method as Compound 65. MS: m/z (obs.) 490.9 [M+1]⁺; ¹H NMR (300 MHz, CDCl₃) δ 6.96-6.70 (m, 1H), 5.43-5.18 (m, 1H), 3.45-2.88 (m, 9H), 1.85-0.53 (m, 27H).

### Preparation of Compound 85

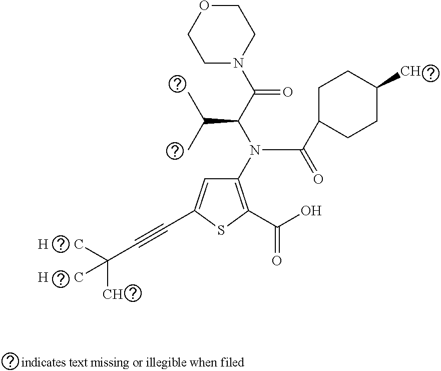

### 5-(3,3-Dimethylbut-1-ynyl)-3-[(trans-4-methylcyclohexanecarbonyl)-[(1S)-2-methyl-1-(morpholine-4-carbonyl)propyl]amino]thiophene-2-carboxylic acid

Compound 85 was prepared in the manner described for 65.

### Preparation of Compound 86

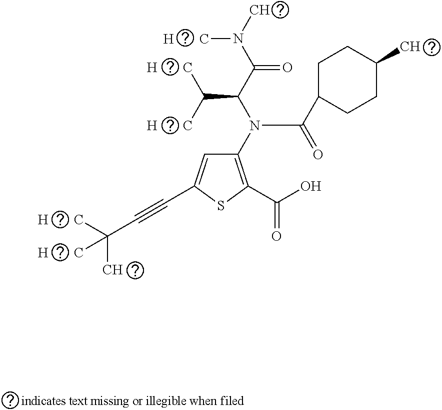

### 5-(3,3-Dimethylbut-1-ynyl)-3-[[(1S)-1-(dimethylcarbamoyl)-2-methyl-propyl]-(trans-4-methylcyclohexanecarbonyl)amino]thiophene-2-carboxylic acid

Compound 86 was prepared in the manner described for 65.

### Preparation of Compound 75

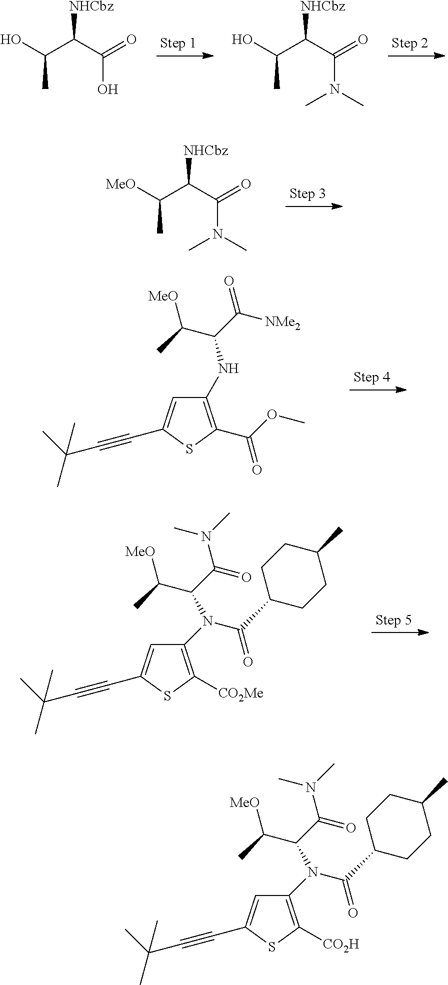

### Benzyl((2R,3R)-1-(dimethylamino)-3-hydroxy-1-oxobutan-2-yl)carbamate

A solution of (2R,3R)-2-(((benzyloxy)carbonyl)amino)-3-hydroxybutanoic acid (340 mg, 1.3 mmol) in DMF (2 mL) was added TBTU (647 mg, 2.0 mmol), DIEA (694 mg, 0.94 mL, 5.4 mmol), N-methylmethanamine hydrochloride (329 mg, 4.0 mmol). The solution was stirred for 16 h at RT, diluted with EtOAc (20 mL), washed with H₂O (15 mL) and brine (15 mL), dried over Na₂SO₄ and concentrated. The residue was purified by silica gel column chromatography, eluting with 0-100% EtOAc/Hex to afford benzyl((2R,3R)-1-(dimethylamino)-3-hydroxy-1-oxobutan-2-yl)carbamate (300 mg, 82%, white solid). MS: m/z (obs.) 280.96 [M+1]⁺; ¹H NMR (300 MHz, CDCl₃) δ 7.45-7.30 (m, 5H), 5.92 (d, J=8.3 Hz, 1H), 5.20-5.02 (m, 2H), 4.61 (ddd, J=21.5, 8.3, 4.2 Hz, 1H), 4.02-3.84 (m, 1H), 3.17 (s, 3H), 2.97 (s, 3H), 1.20 (d, J=6.0 Hz, 3H).

### Step 2

### ((2R,3R)-1-(dimethylamino)-3-methoxy-1-oxobutan-2-yl)carbamate

A solution of benzyl((2R,3R)-1-(dimethylamino)-3-hydroxy-1-oxobutan-2-yl)carbamate (300 mg, 1.1 mmol) in DCM (10 mL) was added Ag₂O (1.2 g, 5.4 mmol) and MeI (1.5 g, 0.7 mL, 11 mmol) at 0° C. under dark and stirred for 3 days at RT. The reaction mixture was filtered though a layer of celite and concentrated. The residue was purified by silica gel chromatography, eluting with 0-70% EtOAc in hexane. This afforded ((2R,3R)-1-(dimethylamino)-3-methoxy-1-oxobutan-2-yl)carbamate (120 mg, 38%). MS: m/z (obs.) 295.1 [M+1]⁺. To a solution of the resulting ((2R,3R)-1-(dimethylamino)-3-methoxy-1-oxobutan-2-yl)carbamate (120 mg, 0.41 mmol) in MeOH (10 mL) was added Pd(OH)₂ (28 mg, 0.2 mmol) and stirred for 1 h under H₂ balloon. The mixture was filtered though a layer of celite. The filtrate was concentrated and carried to the next step. MS: m/z (obs.) 161 [M+1]⁺.

### Step 3

### Methyl 3-(((2R,3R)-1-(dimethylamino)-3-methoxy-1-oxobutan-2-yl)amino)-5-(3,3-dimethylbut-1-yn-1-yl)thiophene-2-carboxylate

A mixture of (2R,3R)-2-amino-3-methoxy-N,N-dimethyl-butanamide (70 mg, 0.44 mmol), cesium carbonate (388 mg, 1.2 mmol), rac-BINAP (50 mg, 0.08 mmol), Pd₂(dba)₃ (36 mg, 0.04 mmol) and methyl 3-bromo-5-(3,3-dimethylbut-1-ynyl)thiophene-2-carboxylate (120 mg, 0.4 mmol) in Dioxane (2 mL) was bubbled N₂ for 30 min and then stirred for 16 h at 95° C. Reaction progress was monitored by LC-MS. On completion the reaction mixture was diluted with EtOAc (20 mL), filtered though a layer of celite. The filtrate was washed with H₂O (15 mL), brine (15 mL), dried over Na₂SO₄, and concentrated. The residue was purified by silica gel chromatography, eluting with 0-50% EtOAc in hexane to afford methyl 3-(((2R,3R)-1-(dimethylamino)-3-methoxy-1-oxobutan-2-yl)amino)-5-(3,3-dimethylbut-1-yn-1-yl)thiophene-2-carboxylate (100 mg, 66%). MS: m/z (obs.) 380.9 [M+1]⁺

### Step 4

### Compound 75, methyl ester

A solution of methyl 5-(3,3-dimethylbut-1-ynyl)-3-[[(1R,2R)-1-(dimethylcarbamoyl)-2-methoxy-propyl]amino]thiophene-2-carboxylate (100 mg, 0.26 mmol) in DCE (3.0 mL) was added Pyridine (208 mg, 0.2 mL, 2.6 mmol), DMAP (3 mg, 0.03 mmol), and trans 4-methylcyclohexanecarbonyl chloride (211 mg, 1.3 mmol) and heated to 100° C. for 24 h. Reaction progress was monitored by LC-MS. On completion the reaction mixture was cooled to RT, diluted with EtOAc (20 mL), washed with sat. NaHCO₃ (15 mL) and concentrated. The residue was purified by silica gel chromatography, eluting with 0-60% EtOAc in hexane. This afforded the desired compound 75, methyl ester (95 mg, 72%). MS: m/z (obs.) 505.2 [M+1]⁺.

### Step 5

### Compound 75

A solution of starting ester (90 mg, 0.18 mmol) in MeOH (2 mL) was added NaOH (0.9 mL of 1 M, 0.9 mmol) and stirred for overnight at RT. The mixture was acidified with HCl to PH=3, concentrated. The residue was purified by silica gel chromatography, eluting with 0-4% MeOH in DCM to afford 75 (49 mg, 53%). MS: m/z (obs.) 490.97 [M+1]⁺; ¹H NMR (300 MHz, CDCl₃) δ 6.80 (s, 1H), 5.27 (dd, J=17.1, 7.9 Hz, 2H), 3.86-2.84 (m, 10H), 1.86-0.54 (m, 25H).

### Preparation of Compound 74

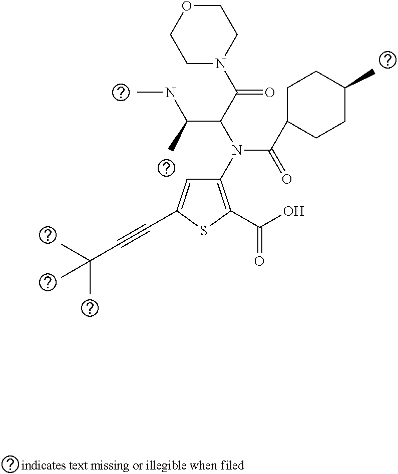

### 5-(3,3-dimethylbut-1-ynyl)-3-[[(1R,2R)-2-methoxy-1-(morpholine-4-carbonyl)propyl]-(trans-4-methylcyclohexanecarbonyl)amino]thiophene-2-carboxylic acid

Compound 74 was prepared in the manner described for compound 75. MS: m/z (obs.) 532.99 [M+1]⁺; ¹H NMR (300 MHz, CDCl₃) δ 6.80 (s, 1H), 5.44-5.23 (m, 1H), 4.00-3.29 (m, 10H), 3.19-2.97 (m, 3H), 1.89-0.42 (m, 25H).

### Preparation of Compound 72

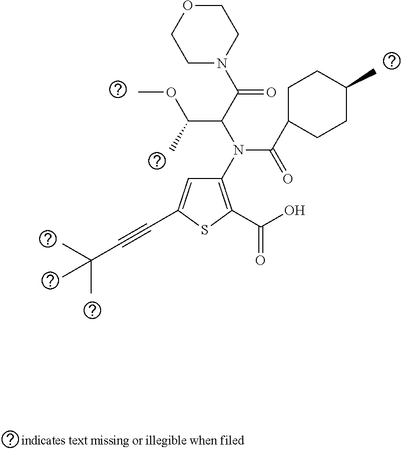

### 5-(3,3-dimethylbut-1-ynyl)-3-[[(1R,2S)-2-methoxy-1-(morpholine-4-carbonyl)propyl]-(trans-4-methylcyclohexanecarbonyl)amino]thiophene-2-carboxylic acid

Compound 72 was prepared in the manner described for compound 75. MS: m/z (obs.) 532.99 [M+1]¹; ¹H NMR (300 MHz, CDCl₃) δ 7.01-6.71 (m, 1H), 5.51-5.20 (m, 2H), 3.97-3.30 (m, 9H), 3.27-2.84 (m, 3H), 1.83-0.56 (m, 27H).

### Preparation of Compound 57

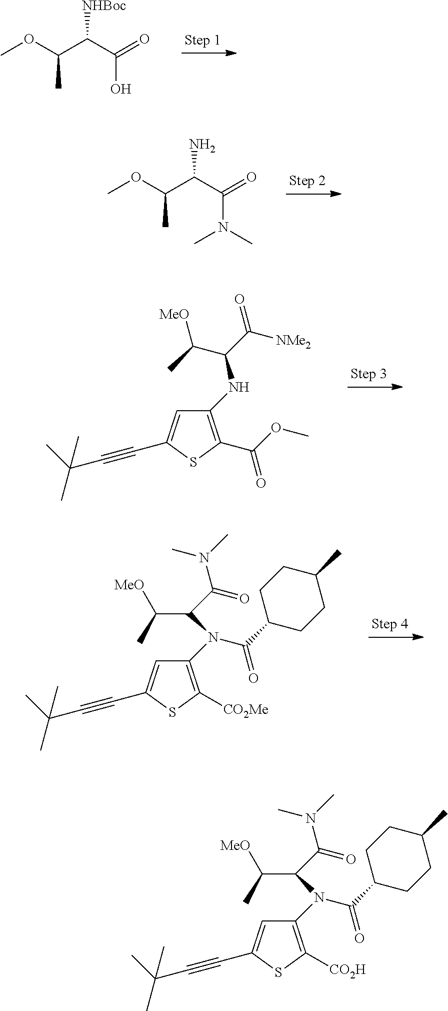

### Step 1

### Tert-butyl((2S,3R)-1-(dimethylamino)-3-methoxy-1-oxobutan-2-yl)carbamate

A solution of (2S,3R)-2-(tert-butoxycarbonylamino)-3-methoxy-butanoic acid (400 mg, 1.7 mmol) in DMF (3 mL) was added DIEA (665 mg, 0.9 mL, 5.1 mmol), HBTU (976 mg, 2.6 mmol) and N-methylmethanamine (580 mg, 0.62 mL of 40% w/w, 5.1 mmol) and stirred overnight at RT. The mixture was diluted with EtOAc (20 mL), washed with H₂O (15 mL), brine (15 mL), dried over Na₂SO₄ and concentrated. The residue was purified by silica gel chromatography, eluting with 0-60% EtOAc in hexane to afford tert-butyl((2S,3R)-1-(dimethylamino)-3-methoxy-1-oxobutan-2-yl)carbamate (350 mg, 1.3 mmol, 78%). To resulting tert-butyl((2S,3R)-1-(dimethylamino)-3-methoxy-1-oxobutan-2-yl)carbamate (350 mg, 1.3 mmol) was added HCl (5 mL of 4.0 M, 20 mmol) in dioxane and stirred for 2 h at RT. The solution was concentrated and used directly in next step.

### Step 2

### Methyl 3-(((2S,3R)-1-(dimethylamino)-3-methoxy-1-oxobutan-2-yl)amino)-5-(3,3-dimethylbut-1-yn-1-yl)thiophene-2-carboxylate

A solution of methyl 3-bromo-5-(3,3-dimethylbut-1-ynyl)thiophene-2-carboxylate (301 mg, 1 mmol) in Dioxane (5 mL) was added (2S,3R)-2-amino-3-methoxy-N,N-dimethyl-butanamide (Hydrochloric Acid (1)) (256 mg, 1.3 mmol), rac-BINAP (125 mg, 0.2 mmol), Pd₂(dba)₃ (92 mg, 0.1 mmol) and dicesium carbonate (978 mg, 3.0 mmol). The solution was bubbled N₂ for 30 min. and heated to 90° C. for 18 h. Reaction progress was monitored by LC-MS. On completion the reaction mixture was cooled to RT, diluted with EtOAc (30 mL), filtered though a layer of celite, concentrated. The residue was purified by silica gel chromatography, eluting with 0-50% EtOAc in hexane to afford methyl 3-(((2S,3R)-1-(dimethylamino)-3-methoxy-1-oxobutan-2-yl)amino)-5-(3,3-dimethylbut-1-yn-1-yl)thiophene-2-carboxylate (280 mg, 73.6%). MS: m/z (obs.) 380.96 [M+1]⁺

### Step 3

### Compound 57, methyl ester

A solution of methyl 5-(3,3-dimethylbut-1-ynyl)-3-[[(1S,2R)-1-(dimethylcarbamoyl)-2-methoxy-propyl]amino]thiophene-2-carboxylate (280 mg, 0.7359 mmol) in DCE (4.119 mL) was added pyridine (582.1 mg, 595.2 μL, 7.359 mmol), DMAP (17.98 mg, 0.1472 mmol) and trans 4-methylcyclohexanecarbonyl chloride (591.2 mg, 3.680 mmol). The mixture was stirred for 24 h at 100° C. Reaction progress was monitored by LC-MS. On completion, the reaction mixture was cooled down to RT, diluted with EtOAc (30 mL), washed with brine (2×20 mL), dried over Na₂SO₄ and concentrated. The residue was purified by silica gel chromatography, eluting with 0-50% EtOAc in hexane to afford compound 57, methyl ester (230 mg, 61.9%). MS: m/z (obs.) 504.96 [M+1]⁺.

### Step 4

### Compound 57

To a solution of starting ester (54 mg, 0.11 mmol) in MeOH (3 mL) was added NaOH (0.33 mL of 1.0 M, 0.33 mmol). The solution was stirred overnight. The reaction was monitored by LC-MS. On completion, the reaction mixture was acidified with 1 M aq HCl and concentrated. The residue was purified by silica gel chromatography eluting with 0-5% MeOH in DCM to afford 57 (117 mg, 71.5%, white solid). MS: m/z (obs.) 490.97. ¹H NMR (300 MHz, CDCl₃) δ 6.90 (s, 1H), 5.25 (dd, J=20.9, 9.5 Hz, 2H), 3.55-3.22 (m, 4H), 3.02 (dd, J=14.8, 10.4 Hz, 6H), 2.12-1.00 (m, 20H), 0.90-0.52 (m, 5H).

### Preparation of Compound 58

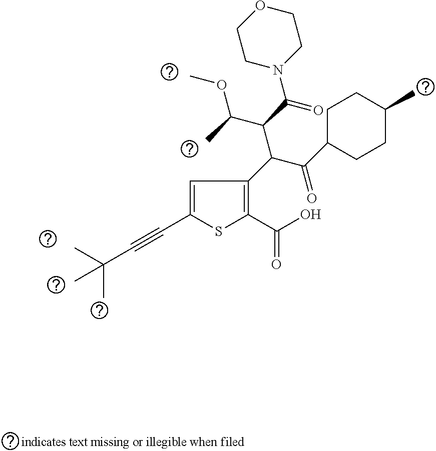

### 5-(3,3-Dimethylbut-1-ynyl)-3-[[(1S,2R)-2-methoxy-1-(morpholine-4-carbonyl)propyl]-(trans-4-methylcyclohexanecarbonyl)amino]thiophene-2-carboxylic acid

Compound 58 was prepared in the manner described for compound 57.

### Preparation of Compound 87

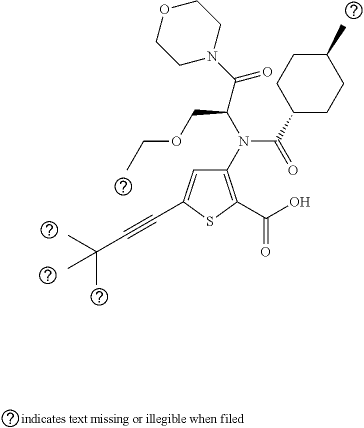 [SMILES: CCOC[C@@H](C(=O)N1CCOCC1)N(c1cc(C#CC(C)(C)C)sc1C(=O)O)C(=O)[C@H]1CC[C@H](C)CC1]

### 5-(3,3-Dimethylbut-1-ynyl)-3-[[(1S)-1-(ethoxymethyl)-2-morpholino-2-oxo-ethyl]-(trans-4-methylcyclohexanecarbonyl)amino]thiophene-2-carboxylic acid

Compound 87 was prepared in the manner described for compound 57.

### Preparation of Compound 88

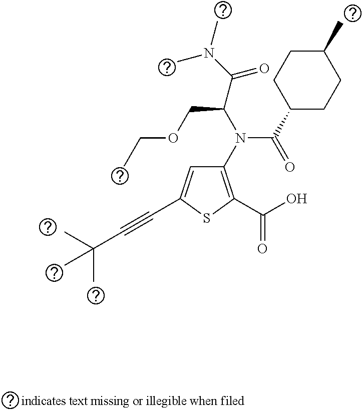 [SMILES: CCOC[C@@H](C(=O)N(C)C)N(c1cc(C#CC(C)(C)C)sc1C(=O)O)C(=O)[C@H]1CC[C@H](C)CC1]

### 3-[[(1S)-2-(Dimethylamino)-1-(ethoxymethyl)-2-oxo-ethyl]-(trans-4-methylcyclohexanecarbonyl)amino]-5-(3,3-dimethylbut-1-ynyl)thiophene-2-carboxylic acid

Compound 88 was prepared in the manner described for compound 57.

### Preparation of Compound 59

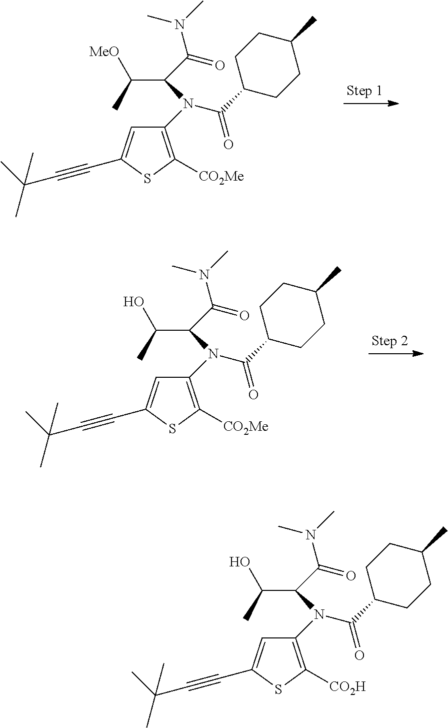

### Step 1

### Compound 59, methyl ester

To a solution of starting methyl ether (66 mg, 0.13 mmol) in DCM (2 mL) at −78° C. was added 1.0 M BBr₃ (0.26 mL, 0.26 mmol) in DCM. The reaction mixture was stirred for 1 h from −78° C. to 0° C. and then quenched with sat. NaHCO₃ solution (1 mL), diluted with EtOAc (20 mL), washed with brine (15 mL), dried over Na₂SO₄ and concentrated to afford desired alcohol (44 mg, 68.7%). MS: m/z (obs.) 490.97 [M+1]⁺.

### Step 2

### Compound 59

To a solution of methyl 3-((1r,4S)—N-((2S,3R)-1-(dimethylamino)-3-hydroxy-1-oxobutan-2-yl)-4-methylcyclohexanecarboxamido)-5-(3,3-dimethylbut-1-yn-1-yl)thiophene-2-carboxylate (54 mg, 0.11 mmol) in MeOH (3 mL) was added 1.0 M aqueous NaOH (0.33 mL, 0.33 mmol). The solution was stirred for overnight, acidified with 6 M aq HCl and concentrated. The residue was purified by silica gel chromatography, eluting with 0-5% MeOH in DCM to afford 59 (26 mg, 47%, white solid). MS: m/z (obs.) 476.97 [M+1]⁺; ¹H NMR (300 MHz, CDCl₃) δ 7.02-6.87 (m, 1H), 5.61 (dd, J=43.5, 7.6 Hz, 2H), 4.19-3.90 (m, 2H), 3.24 (s, 4H), 3.16 (s, 3H), 2.97 (d, J=18.4 Hz, 3H), 2.83 (s, 2H), 2.06 (d, J=9.8 Hz, 2H), 1.89-1.06 (m, 11H), 0.95-0.57 (m, 6H).

### Preparation of Compound 60

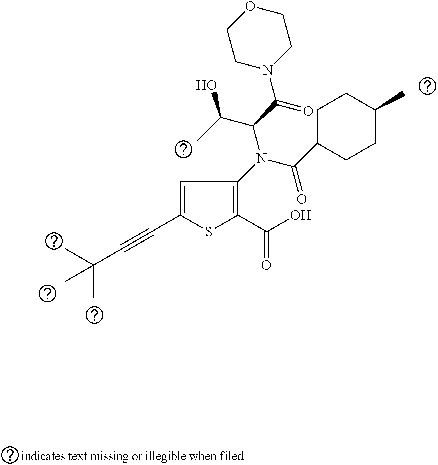

### 5-(3,3-Dimethylbut-1-ynyl)-3-[[(1S,2R)-2-hydroxy-1-(morpholine-4-carbonyl)propyl]-(trans-4-methylcyclohexanecarbonyl)amino]thiophene-2-carboxylic acid

Compound 60 was prepared in the manner described for compound 59.

### Preparation of Compound 33

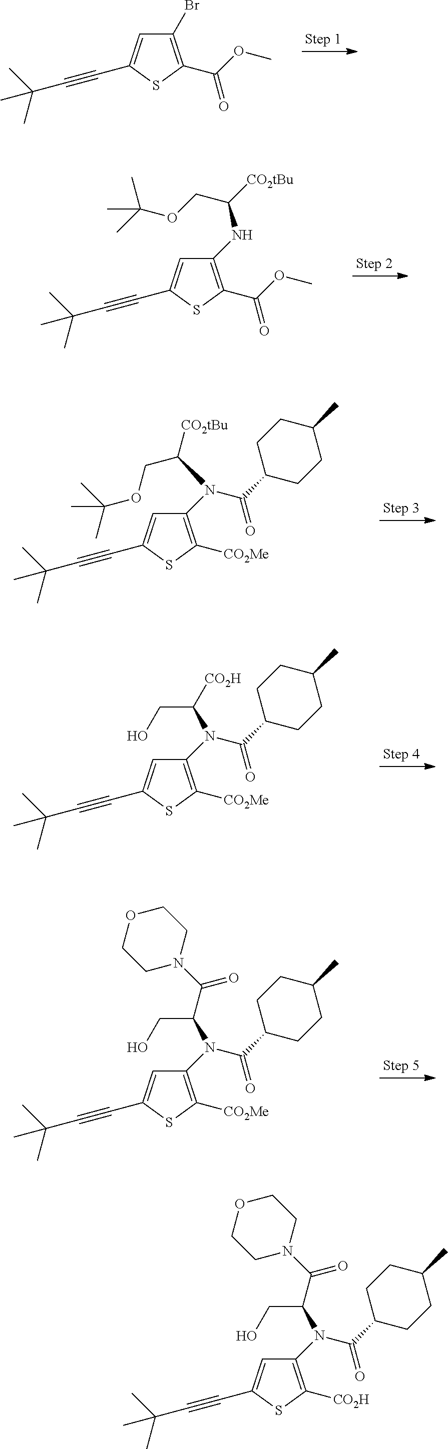

### Step 1

### (S)-methyl 3-((1,3-di-tert-butoxy-1-oxopropan-2-yl)amino)-5-(3,3-dimethylbut-1-yn-1-yl)thiophene-2-carboxylate

A mixture of methyl 3-bromo-5-(3,3-dimethylbut-1-ynyl)thiophene-2-carboxylate (1 g, 3.3 mmol), rac-BINAP (414 mg, 0.66 mmol), tert-butyl (2S)-2-amino-3-tert-butoxy-propanoate hydrochloride (1.0 g, 4.0 mmol), Pd₂(dba)₃ (304 mg, 0.33 mmol) and dicesium carbonate (3.25 g, 10 mmol) was degassed and filled with N₂. The reaction mixture was added 1,4-dioxane (15 mL), bubbled N₂ for 30 min and stirred for 24 h at 90° C. Reaction progress was monitored by LC-MS. On completion, the reaction mixture was diluted with EtOAc (50 mL), filtered though a layer of celite and concentrated. The residue was purified by silica gel chromatography, eluting with 0-10% EtOAc in hexane to afford (S)-methyl 3-((1,3-di-tert-butoxy-1-oxopropan-2-yl)amino)-5-(3,3-dimethylbut-1-yn-1-yl)thiophene-2-carboxylate (1.0 g, 68.8%, white solid). MS: m/z (obs.) 438.0 [M+1]⁺.

### Step 2

To a solution of methyl 3-[[(1S)-2-tert-butoxy-1-(tert-butoxymethyl)-2-oxo-ethyl]amino]-5-(3,3-dimethylbut-1-ynyl)thiophene-2-carboxylate (1.0 g, 2.3 mmol) in DCE (2 mL) was added pyridine (1.08 g, 1.1 mL, 13.71 mmol), DMAP (28 mg, 0.23 mmol) and trans-4-methylcyclohexanecarbonyl chloride (1.47 g, 9.1 mmol) and reflux for 24 h. Reaction progress was monitored by LC-MS. On completion, the reaction mixture was diluted with EtOAc (50 mL), washed with H₂O (30 mL), brine (30 mL), dried over Na₂SO₄ and concentrated. The residue was purified by silica gel chromatography eluting with 0-50% EtOAc in hexane to afford the desired amide (1.1 g, 85.7%). MS: m/z (obs.) 562 [M+1]⁺.

### Step 3

A solution of starting alkoxy ester (1.0 g, 1.78 mmol) in DCM (30 ML) was added TFA (609 mg, 0.41 mL, 5.3 mmol) and stirred overnight at RT. Reaction was monitored by LC-MS. On completion, the reaction mixture was concentrated. The residue was purified from column 0-100% EtOAc in hexane to afford the desired hydroxy acid (580 mg, 72%). MS: m/z (obs.) 450 [M+1]⁺.

### Step 4

### Compound 33, methyl ester

To a solution of the hydroxy acid prepared via step 3 (81 mg, 0.18 mmol) in DMF (2.0 mL) was added HBTU (104 mg, 0.28 mmol), DIEA (72 mg, 96 μL, 0.54 mmol) and morpholine (48 mg, 0.54 mmol) and stirred for 2 h at rt. The reaction progress was monitored by LC-MS. On completion, the reaction mixture was diluted with EtOAc (20 mL), washed with H₂O (15 mL), brine (15 mL), dried over Na₂SO₄ and concentrated. The residue was purified by silica gel chromatography eluting with 0-100% EtOAc in hexane to afford compound 33, methyl ester (52 mg, 53.6%). MS: m/z (obs.) 519.0 [M+1]⁺.

### Step 5

### Compound 33

To a solution of compound 33, methyl ester (50 mg, 0.1 mmol) in MeOH (5 mL) was added IM aq NaOH (0.5 mL, 0.5 mmol) and stirred overnight. On completion, the reaction mixture was acidified with 6M aq. HCl to PH=3 and concentrated. The residue was purified by silica gel chromatography, eluting with 0-5% MeOH in DCM to afford 33 (25 mg, 46%, white solid). MS: m/z (obs.) 505.0 [M+1]⁺.

### Preparation of Compound 34

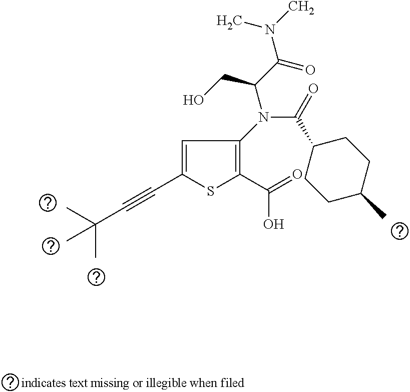 [SMILES: CN(C)C(=O)[C@H](CO)N(c1cc(C#CC(C)(C)C)sc1C(=O)O)C(=O)[C@H]1CC[C@H](C)CC1]

### 3-[[(1S)-2-(dimethylamino)-1-(hydroxymethyl)-2-oxo-ethyl]-(trans-4-methylcyclohexanecarbonyl)amino]-5-(3,3-dimethylbut-1-ynyl)thiophene-2-carboxylic acid

Compound 34 was prepared in the manner described for compound 33.

### Preparation of Compound 112

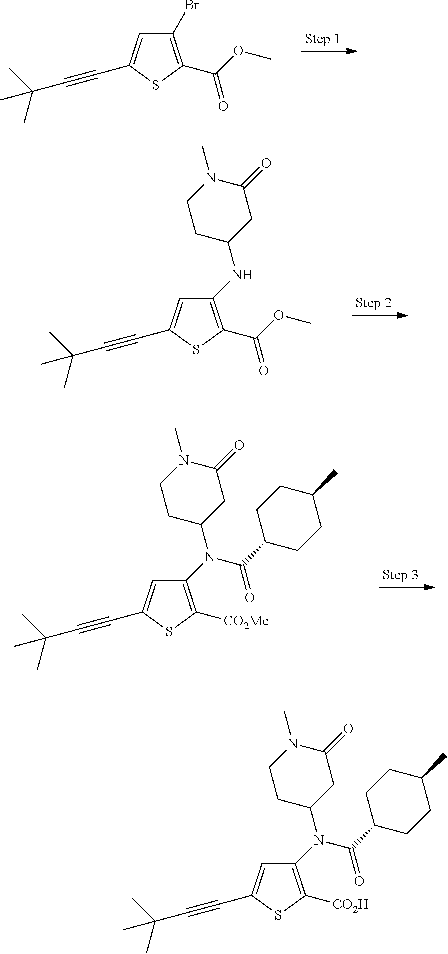

### Step 1

### Methyl 5-(3,3-dimethylbut-1-yn-1-yl)-3-((1-methyl-2-oxopiperidin-4-yl)amino)thiophene-2-carboxylate

To a solution of methyl 3-bromo-5-(3,3-dimethylbut-1-ynyl)thiophene-2-carboxylate (200 mg, 0.66 mmol), 4-amino-1-methylpiperidin-2-one (102 mg, 0.8 mmol), cesium carbonate (649 mg, 1.99 mmol), rac-BINAP (83 mg, 0.13 mmol) and Pd₂(dba)₃ (61 mg, 0.07 mmol) in Dioxane (3.5 mL) was bubbled N₂ for 30 min and stirred for 18 h at 95° C. Reaction was monitored by LC-MS. On completion, the reaction mixture was diluted with EtOAc (30 mL), filtered though a layer of celite and Concentrated. The residue was purified by silica gel chromatography, eluting with 0-80% EtOAc in hexane to afford methyl 5-(3,3-dimethylbut-1-yn-1-yl)-3-((1-methyl-2-oxopiperidin-4-yl)amino)thiophene-2-carboxylate (140 mg, 60.5%). MS: m/z (obs.) 349.0 [M+1]⁺.

### Step 2

### Compound 112, methyl ester

A mixture of methyl 5-(3,3-dimethylbut-1-yn-1-yl)-3-((1-methyl-2-oxopiperidin-4-yl)amino)thiophene-2-carboxylate (140 mg, 0.40 mmol), pyridine (318 mg, 325 μL, 4.0 mmol), DMAP (5 mg, 0.04 mmol) and trans-4-methylcyclohexanecarbonyl chloride (323 mg, 2.0 mmol) in DCE (2 mL) was reflux overnight. Reaction was monitored by LC-MS. On completion, the reaction mixture was diluted with EtOAc (30 mL), washed with sat. NaHCO₃ (15 mL), dried over Na₂SO₄ and concentrated. The residue was purified by silica gel chromatography, eluting with 0-5% MeOH in DCM to afford compound 112, methyl ester (100 mg, 56.3%). MS: m/z (obs.) 444.9 [M+1]⁺.

### Step 3

### Compound 112

To starting methyl ester (100 mg, 0.22 mmol) in MeOH (3 mL) was added 1M aq NaOH (2.0 mL, 2.0 mmol) and stirred overnight. Reaction progress was monitored by LC-MS. On completion, the reaction mixture was acidified with 1M aq HCl to PH=3 and concentrated. The residue was purified by silica gel chromatography, eluting with 0-10% MeOH in DCM. This afforded 112 (59 mg, 58.5%). MS: m/z (obs.) 458.9 [M+1]⁺; ¹H NMR (300 MHz, CDCl₃) δ 6.80 (d, J=18.4 Hz, 1H), 5.10 (s, 1H), 3.61-3.10 (m, 3H), 2.93-2.79 (m, 4H), 2.09-1.90 (m, 5H), 1.87-1.46 (m, 6H), 1.35-1.04 (m, 9H), 0.86-0.76 (m, 5H).

### Preparation of Compound 8

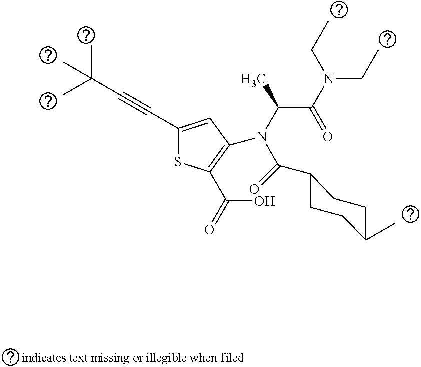 [SMILES: CCN(CC)C(=O)[C@H](C)N(C(=O)C1CCC(C)CC1)c1cc(C#CC(C)(C)C)sc1C(=O)O]

### 3-[[(1S)-2-(diethylamino)-1-methyl-2-oxo-ethyl]-(trans-4-methylcyclohexanecarbonyl)amino]-5-(3,3-dimethylbut-1-ynyl)thiophene-2-carboxylic acid

Compound 8 was prepared in the manner described for 82.

### Preparation of Compound 9

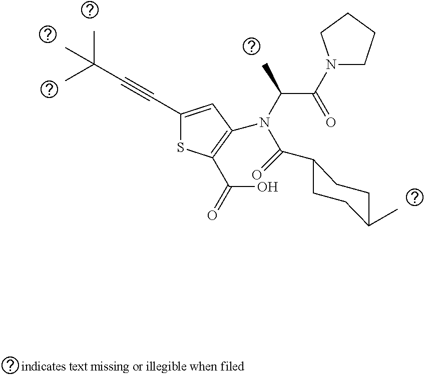 [SMILES: CC1CCC(C(=O)N(c2cc(C#CC(C)(C)C)sc2C(=O)O)[C@@H](C)C(=O)N2CCCC2)CC1]

### 5-(3,3-Dimethylbut-1-ynyl)-3-[(trans-4-methylcyclohexanecarbonyl)-[(1S)-1-methyl-2-oxo-2-pyrrolidin-1-yl-ethyl]amino]thiophene-2-carboxylic acid

Compound 9 was prepared in the manner described for 82.

### Preparation of Compound 18

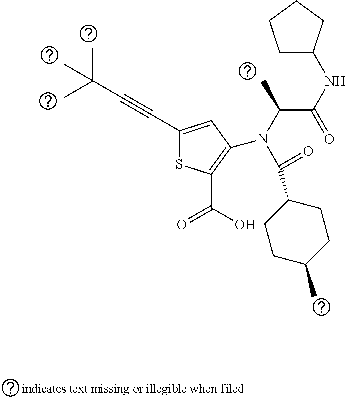 [SMILES: C[C@@H](C(=O)NC1CCCC1)N(c1cc(C#CC(C)(C)C)sc1C(=O)O)C(=O)[C@H]1CC[C@H](C)CC1]

### 3-[[(1S)-2-(Cyclopentylamino)-1-methyl-2-oxo-ethyl]-(trans-4-methylcyclohexanecarbonyl)amino]-5-(3,3-dimethylbut-1-ynyl)thiophene-2-carboxylic acid

Compound 18 was prepared in the manner described for 82.

### Preparation of Compound 19

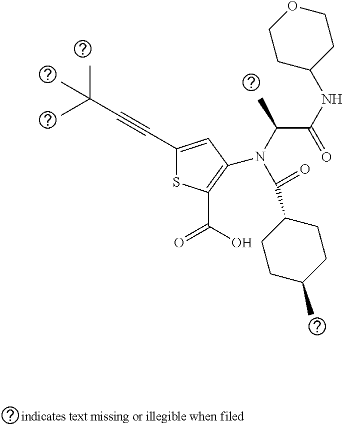 [SMILES: C[C@@H](C(=O)NC1CCOCC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-methyl-2-oxo-2-(tetrahydropyran-4-ylamino)ethyl]amino]thiophene-2-carboxylic acid

Compound 19 was prepared in the manner described for 82.

### Preparation of Compound 20

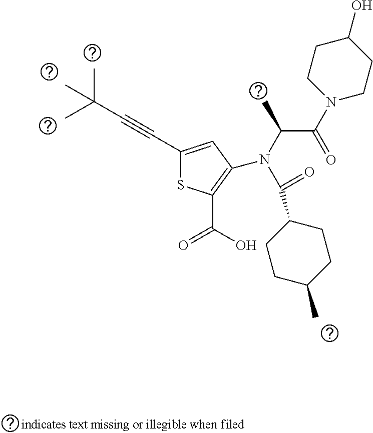 [SMILES: C[C@@H](C(=O)N1CCC(O)CC1)N(c1cc(C#CC(C)(C)C)sc1C(=O)O)C(=O)[C@H]1CC[C@H](C)CC1]

### 5-(3,3-Dimethylbut-1-ynyl)-3-[[(1S)-2-(4-hydroxy-1-piperidyl)-1-methyl-2-oxo-ethyl]-(trans-4-methylcyclohexanecarbonyl)amino]thiophene-2-carboxylic acid

Compound 20 was prepared in the manner described for 82.

### Preparation of Compound 29

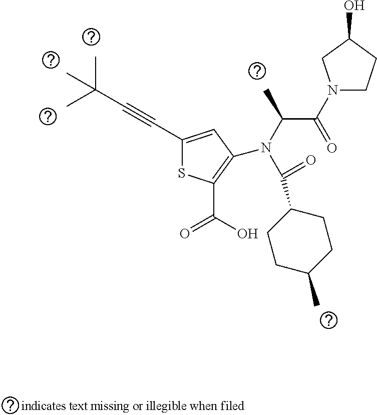 [SMILES: C[C@@H](C(=O)N1CC[C@H](O)C1)N(c1cc(C#CC(C)(C)C)sc1C(=O)O)C(=O)[C@H]1CC[C@H](C)CC1]

### 5-(3,3-Dimethylbut-1-ynyl)-3-[[(1S)-2-[(3S)-3-hydroxypyrrolidin-1-yl]-1-methyl-2-oxo-ethyl]-(trans-4-methylcyclohexanecarbonyl)amino]thiophene-2-carboxylic acid

Compound 29 was prepared in the manner described for 82.

### Preparation of Compound 30

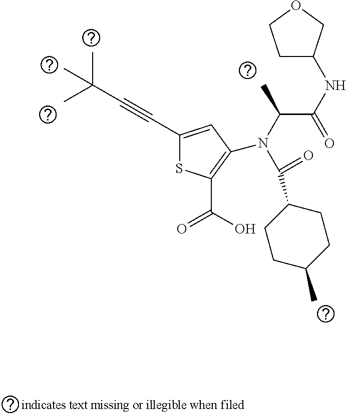 [SMILES: C[C@@H](C(=O)NC1CCOC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-methyl-2-oxo-2-(tetrahydrofuran-3-ylamino)ethyl]amino]thiophene-2-carboxylic acid

Compound 30 was prepared in the manner described for 82.

### Preparation of Compound 31

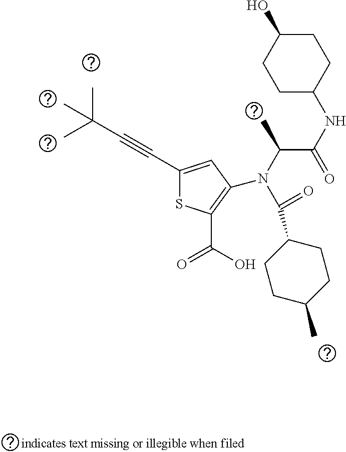

### 5-(3,3-Dimethylbut-1-ynyl)-3-[[(1S)-2-[(4-hydroxycyclohexyl)amino]-1-methyl-2-oxo-ethyl]-(trans-4-methylcyclohexanecarbonyl)amino]thiophene-2-carboxylic acid

Compound 31 was prepared in the manner described for 82.

### Preparation of Compound 32

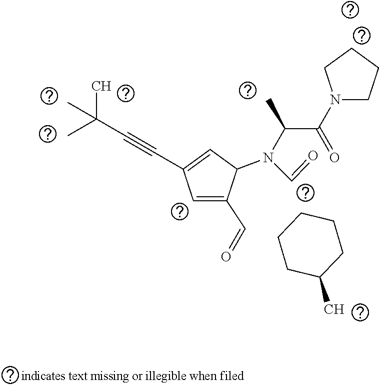

### 5-(3,3-Dimethylbut-1-ynyl)-3-[[(1S)-2-[(3R)-3-hydroxypyrrolidin-1-yl]-1-methyl-2-oxo-ethyl]-(trans-4-methylcyclohexanecarbonyl)amino]thiophene-2-carboxylic acid

Compound 32 was prepared in the manner described for 82.

### Preparation of Compound 37

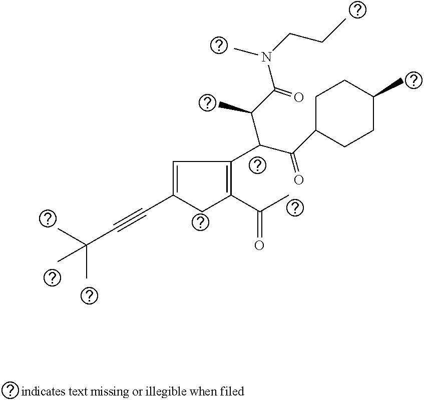

### 5-(3,3-Dimethylbut-1-ynyl)-3-[[(1S)-2-[2-hydroxyethyl(methyl)amino]-1-methyl-2-oxo-ethyl]-(trans-4-methylcyclohexanecarbonyl)amino]thiophene-2-carboxylic acid

Compound 37 was prepared in the manner described for 82.

### Preparation of Compound 38

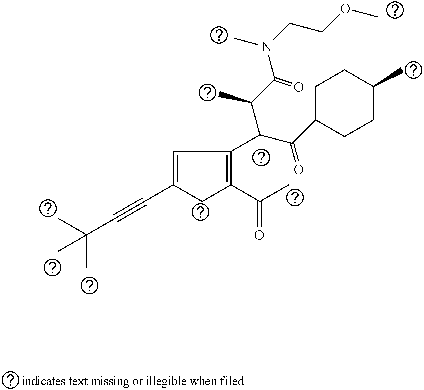

### 5-(3,3-Dimethylbut-1-ynyl)-3-[[(1S)-2-[2-methoxyethyl(methyl)amino]-1-methyl-2-oxo-ethyl]-(trans-4-methylcyclohexanecarbonyl)amino]thiophene-2-carboxylic acid

Compound 38 was prepared in the manner described for 82.

### Preparation of Compound 39

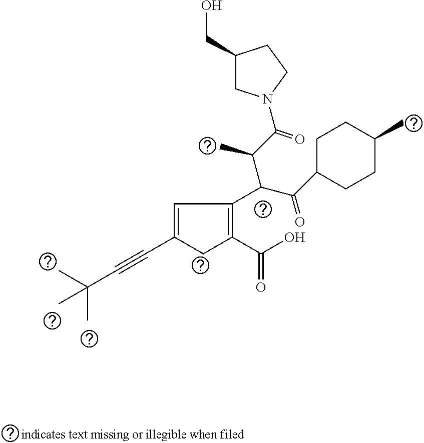

### 5-(3,3-Dimethylbut-1-ynyl)-3-[[(1S)-2-[(3S)-3-(hydroxymethyl)pyrrolidin-1-yl]-1-methyl-2-oxo-ethyl]-(trans-4-methylcyclohexanecarbonyl)amino]thiophene-2-carboxylic acid

Compound 39 was prepared in the manner described for 82.

### Preparation of Compound 40

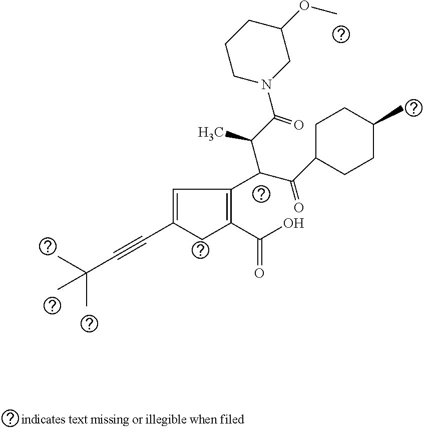

### 5-(3,3-Dimethylbut-1-ynyl)-3-[[(1S)-2-(3-methoxy-1-piperidyl)-1-methyl-2-oxo-ethyl]-(trans-4-methylcyclohexanecarbonyl)amino]thiophene-2-carboxylic acid

Compound 40 was prepared in the manner described for 82.

### Preparation of Compound 47

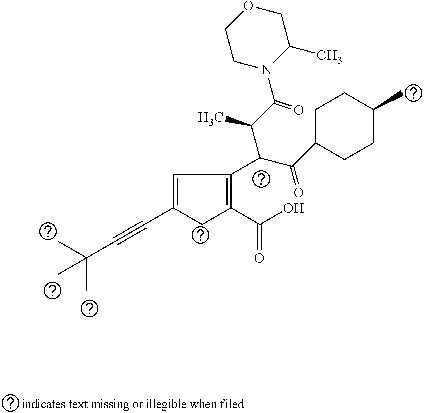

### 5-(3,3-dimethylbut-1-ynyl)-3-[(trans-4-methylcyclohexanecarbonyl)-[(1S)-1-methyl-2-(3-methylmorpholin-4-yl)-2-oxo-ethyl]amino]thiophene-2-carboxylic acid

Compound 47 was prepared in the manner described for 82.

### Preparation of Compound 48

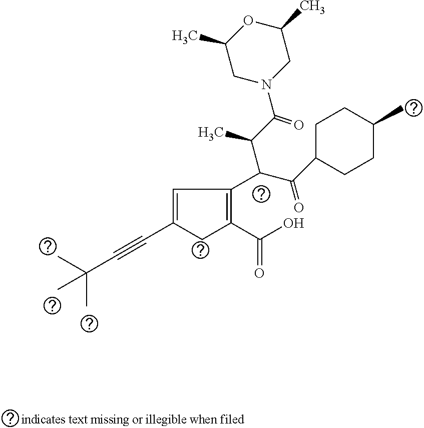

### 5-(3,3-Dimethylbut-1-ynyl)-3-[[(1S)-2-[(2R,6S)-2,6-dimethylmorpholin-4-yl]-1-methyl-2-oxo-ethyl]-(trans-4-methylcyclohexanecarbonyl)amino]thiophene-2-carboxylic acid

Compound 48 was prepared in the manner described for 82.

### Preparation of Compound 49

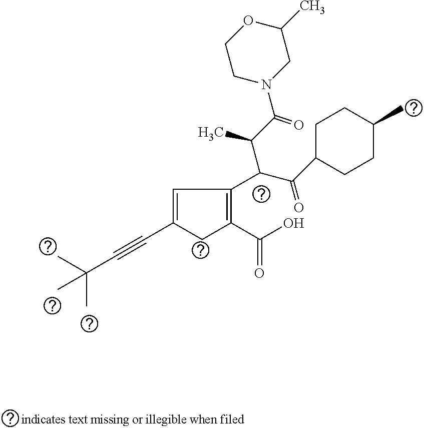

### 5-(3,3-Dimethylbut-1-ynyl)-3-[(trans-4-methylcyclohexanecarbonyl)-[(1S)-1-methyl-2-(2-methylmorpholin-4-yl)-2-oxo-ethyl]amino]thiophene-2-carboxylic acid

Compound 49 was prepared in the manner described for 82.

### Preparation of Compound 50

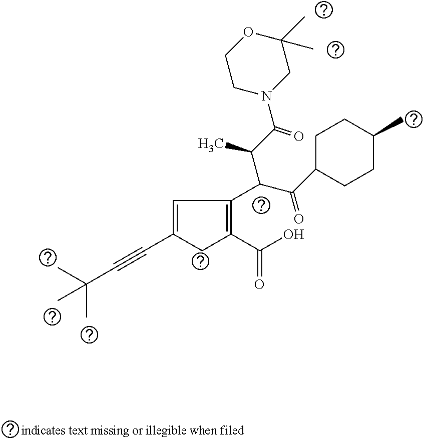

### 5-(3,3-Dimethylbut-1-ynyl)-3-[[(1S)-2-(2,2-dimethylmorpholin-4-yl)-1-methyl-2-oxo-ethyl]-(trans-4-methylcyclohexanecarbonyl)amino]thiophene-2-carboxylic acid

Compound 50 was prepared in the manner described for 82.

### Preparation of Compound 51

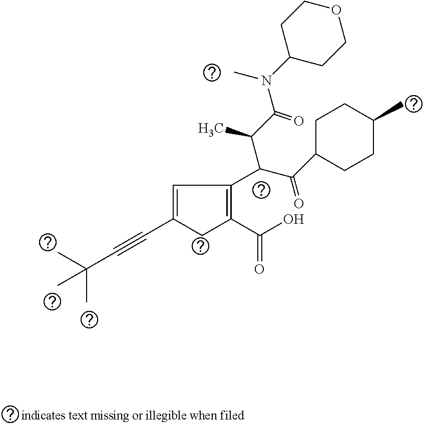

### 5-(3,3-Dimethylbut-1-ynyl)-3-[(trans-4-methylcyclohexanecarbonyl)-[(1S)-1-methyl-2-[methyl(tetrahydropyran-4-yl)amino]-2-oxo-ethyl]amino]thiophene-2-carboxylic acid

Compound 51 was prepared in the manner described for 82.

### Preparation of Compound 52

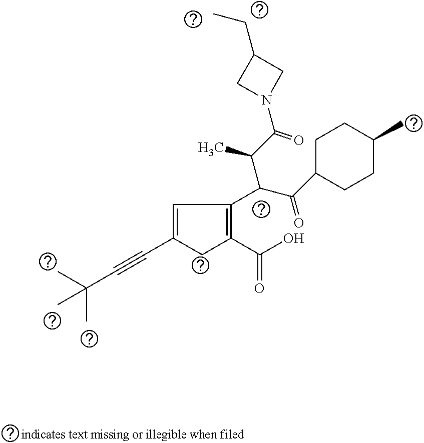

### 5-(3,3-Dimethylbut-1-ynyl)-3-[[(1S)-2-(3-methoxyazetidin-1-yl)-1-methyl-2-oxo-ethyl]-(trans-4-methylcyclohexanecarbonyl)amino]thiophene-2-carboxylic acid

Compound 52 was prepared in the manner described for 82.

### Preparation of Compound 53

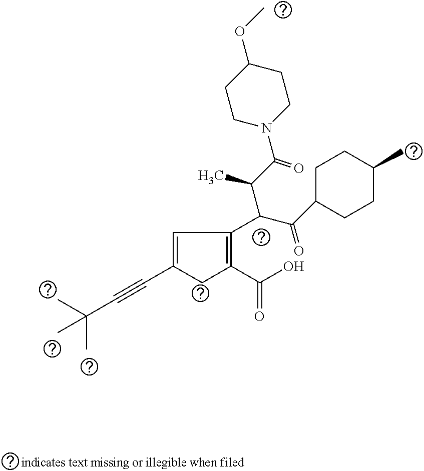

### 5-(3,3-Dimethylbut-1-ynyl)-3-[[(1S)-2-(4-methoxy-1-piperidyl)-1-methyl-2-oxo-ethyl]-(trans-4-methylcyclohexanecarbonyl)amino]thiophene-2-carboxylic acid

Compound 53 was prepared in the manner described for 82.

### Preparation of Compound 54

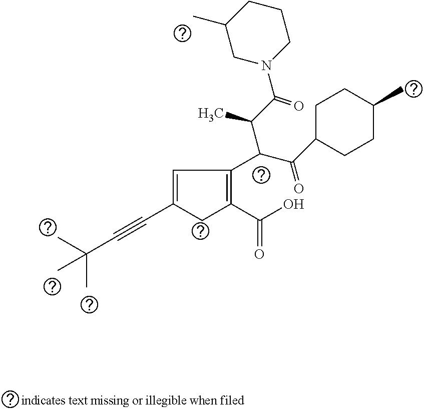

### 5-(3,3-Dimethylbut-1-ynyl)-3-[[(1S)-2-(3-hydroxy-1-piperidyl)-1-methyl-2-oxo-ethyl]-(trans-4-methylcyclohexanecarbonyl)amino]thiophene-2-carboxylic acid

Compound 54 was prepared in the manner described for 82.

### Preparation of Compound 66

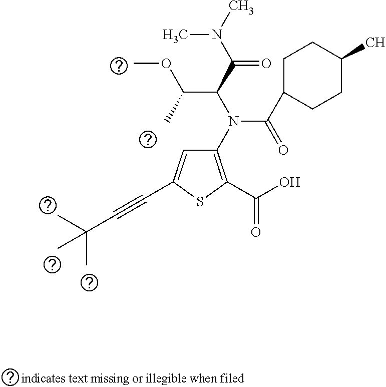

### 5-(3,3-Dimethylbut-1-ynyl)-3-[[(1S,2S)-1-(dimethylcarbamoyl)-2-methoxy-propyl]-(trans-4-methylcyclohexanecarbonyl)amino]thiophene-2-carboxylic acid

Compound 66 was prepared in the manner described for 65.

### Preparation of Compound 76

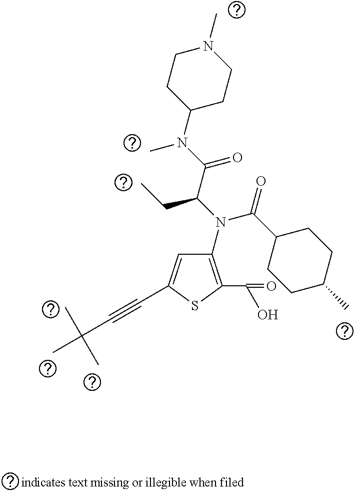

### 5-(3,3-Dimethylbut-1-ynyl)-3-[(trans-4-methylcyclohexanecarbonyl)-[(1S)-1-[methyl-(1-methyl-4-piperidyl)carbamoyl]propyl]amino]thiophene-2-carboxylic acid

Compound 76 was prepared in the manner described for 82.

### Preparation of Compound 77

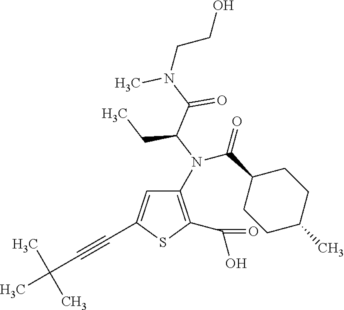 [SMILES: CC[C@@H](C(=O)N(C)CCO)N(c1cc(C#CC(C)(C)C)sc1C(=O)O)C(=O)[C@H]1CC[C@H](C)CC1]

### 5-(3,3-Dimethylbut-1-ynyl)-3-[[(1S)-1-[2-hydroxyethyl(methyl)carbamoyl]propyl]-(trans-4-methylcyclohexanecarbonyl)amino]thiophene-2-carboxylic acid

Compound 77 was prepared in the manner described for 82.

### Preparation of Compound 80

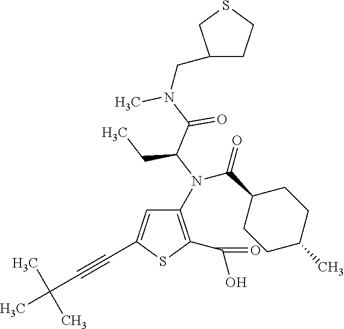 [SMILES: CC[C@@H](C(=O)N(C)CC1CCSC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-[methyl(tetrahydrofuran-3-ylmethyl)carbamoyl]propyl]amino]thiophene-2-carboxylic acid

Compound 80 was prepared in the manner described for 82.

### Preparation of Compound 81

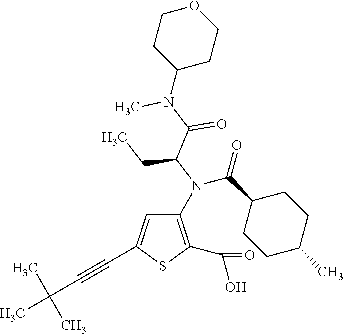 [SMILES: CC[C@@H](C(=O)N(C)C1CCOCC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-[methyl(tetrahydropyran-4-yl)carbamoyl]propyl]amino]thiophene-2-carboxylic acid

Compound 81 was prepared in the manner described for 82.

### Preparation of Compound 84

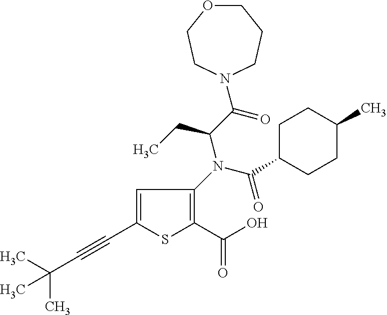 [SMILES: CC[C@@H](C(=O)N1CCCOCC1)N(c1cc(C#CC(C)(C)C)sc1C(=O)O)C(=O)[C@H]1CC[C@H](C)CC1]

### 5-(3,3-Dimethylbut-1-ynyl)-3-[(trans-4-methylcyclohexanecarbonyl)-[(1S)-1-(1,4-oxazepane-4-carbonyl)propyl]amino]thiophene-2-carboxylic acid

Compound 84 was prepared in the manner described for 82.

### Preparation of Compound 89

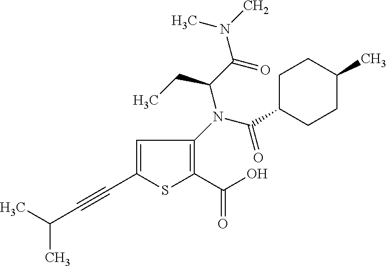 [SMILES: CC[C@@H](C(=O)N(C)C)N(c1cc(C#CC(C)C)sc1C(=O)O)C(=O)[C@H]1CC[C@H](C)CC1]

### 3-[[(1S)-1-(dimethylcarbamoyl)propyl]-(trans-4-methylcyclohexanecarbonyl)amino]-5-(3-methylbut-1-ynyl)thiophene-2-carboxylic acid

Compound 89 was prepared in the manner described for 82.

### Preparation of Compound 90

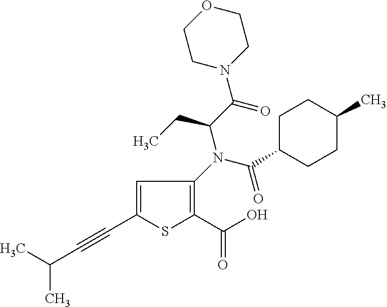 [SMILES: CC[C@@H](C(=O)N1CCOCC1)N(c1cc(C#CC(C)C)sc1C(=O)O)C(=O)[C@H]1CC[C@H](C)CC1]

### 5-(3-Methylbut-1-ynyl)-3-[(trans-4-methylcyclohexanecarbonyl)-[(1S)-1-(morpholine-4-carbonyl)propyl]amino]thiophene-2-carboxylic acid

Compound 90 was prepared in the manner described for 82.

### Preparation of Compound 73

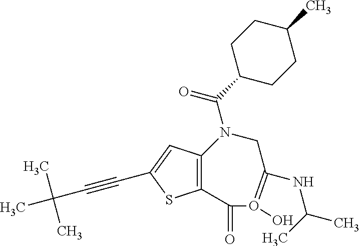

### 5-(3,3-dimethylbut-1-ynyl)-3-[[2-(isopropylamino)-2-oxo-ethyl]-(trans-4-methylcyclohexanecarbonyl)amino]thiophene-2-carboxylic acid

Compound 73 was prepared in the manner described for compound 5. MS: m/z (obs.) 447.4 [M+H]⁺.

### Preparation of Compound 156

### Step 1

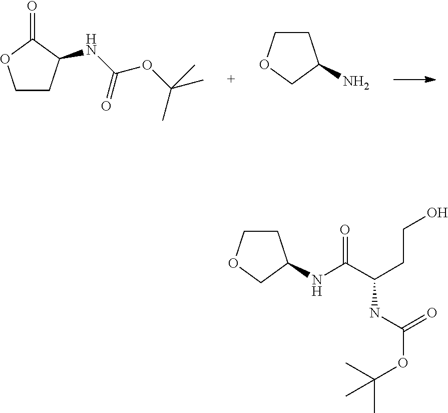 [SMILES: CC(C)(C)OC(=O)N[C@H]1CCOC1=O ; N[C@@H]1CCOC1 ; CC(C)(C)OC(=O)N[C@@H](CCO)C(=O)N[C@@H]1CCOC1]

### Tert-butyl N-[(1S)-3-hydroxy-1-[[(3R)-tetrahydrofuran-3-yl]carbamoyl]propyl]carbamate

To a stirred solution of (S)-tetrahydrofuran-2-amine (2.53 g, 28.8 mmol) in dry DCM (90 mL), was added trimethyl aluminum (55.3 mL, 57.5 mmol) at 0 deg. C. under argon and stirred for 15 min when the lactone in DCM was added and the reaction stirred at RT for 18 h. Reaction progress was monitored by TLC. On completion, the reaction mixture was poured into 10% citric acid and extracted with DCM (2×150 mL) and washed with sat'd. aq. NaHCO₃ solution. (100 mL), water (50 mL), brine (50 mL), dried over anhydrous Na₂SO₄, filtered, then concentrated. The crude compound was purified by column chromatography (100-200 mesh silica gel; 4% MeOH—CHCl₃ as eluent) to afford the desired product (4.2 g, 76%, off white solid). TLC (10% MeOH—CHCl₃) R_{f}=0.37. LC-MS conditions: column HALO C18 (2.1×100 mm, 2.7 μM), mobile phases A: 5 mM NH₄OAc, B: acetonitrile; t/% B: 0 min/3%, 1.5/45, 2.5/45, 3.2/95, 4.7/9, 5/03; flow rate: 0.6 mL/min, sample diluent: MeOH. MS: m/z (obs.): 289.5 [M+H]⁺. Retention time: 1.3 min. ¹H NMR (400 MHz, DMSO-d6): δ 7.94 (d, J=6.8 Hz, 1H, exchanged with D₂O), 6.77 (d, J=8 Hz, J^{1H}, exchanged with D₂O), 4.22 (d, J=3.2 Hz) 3.98-3.94 (d, J=12 Hz, 1H), 3.80-3.65 (m, 2H), 3.63-3.32 (m, 2H), 2.11-2.02 (m, 1H), 1.73-1.61 (m, 3H), 1.37 (s, 9H).

### Step 2

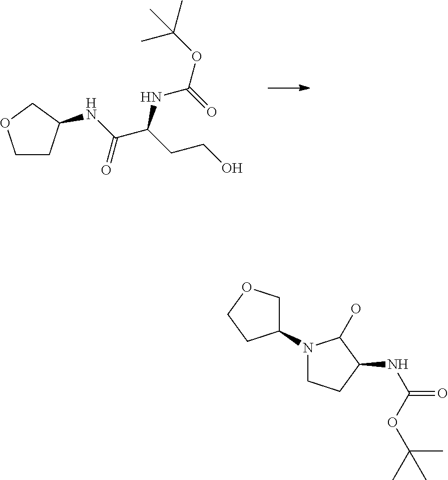 [SMILES: CC(C)(C)OC(=O)N[C@@H](CCO)C(=O)N[C@H]1CCOC1 ; CC(C)(C)OC(=O)N[C@H]1CCN([C@H]2CCOC2)C1O]

### Tert-butyl N-[(3S)-2-oxo-1-[(3S)-tetrahydrofuran-3-yl]pyrrolidin-3-yl]carbamate

To a stirred solution of ditertiary butylazodicarboxylate (959 mg, 4.12 mmol) in dry THF (9 mL) was added tri n-butyl phosphine (2.47 mL, 10.4 mmol) at RT and stirred for 15 min and then added hydroxy amide (600 mg, 2.08 mmol) was added at 0 deg. C. The reaction was stirred for 18 h, and reaction progress monitored by TLC. Upon completion, the reaction mixture was poured into saturated aq. NaHCO₃ solution and extracted with DCM (2×150 mL) and washed with sat. aq. NaHCO₃ (100 mL), water (50 mL), brine (50 mL), dried over Na₂SO₄, then filtered and concentrated. The crude compound was purified by column chromatography (100-200 mesh silica gel; 3.5% MeOH—CHCl₃ as eluent) to afford the desired product (520 mg, 92%, white solid). TLC (10% MeOH—CHCl₃) R_{f}=0.53. LC-ELSD conditions: column HALO C18 (2.1×100 mm, 2.6 μM), mobile phases A: 5 mM NH₄OAc, B: acetonitrile; t/% B: 0 min/3%, 0.5/3, 2/90, 2.5/90, 3.0/100, 3.5/3, 4/3; flow rate: 0.6 mL/min, sample diluent: MeOH, MS: m/z (obs.): 271.1 [M+H]⁺. Retention time: 1.6 min. ¹H NMR (400 MHz, DMSO-d6): δ 7.06 (d, J=9.2 Hz, 1H, exchanged with D2O), 4.59-4.57 (m, 1H), 4.08-4.04 (m, 1H), 3.87-3.81 (m, 1H), 3.64-3.60 (m, 2H), 3.29-3.19 (m, 1H), 2.23-2.16 (m, 1H), 2.09-2.00 (m, 1H), 1.90-1.82 (m, 1H), 1.78-1.69 (m, 1H), 1.61-1.54 (m, 3H), 1.44.

### Step 3

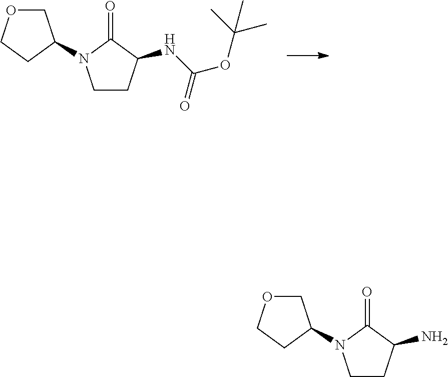 [SMILES: CC(C)(C)OC(=O)N[C@H]1CCN([C@H]2CCOC2)C1=O ; N[C@H]1CCN([C@H]2CCOC2)C1=O]

### (3S)-3-amino-1-[(3S)-tetrahydrofuran-3-yl]pyrrolidin-2-one

To a stirred solution of tert-butyl N-[(3S)-2-oxo-1-[(3S)-tetrahydrofuran-3-yl]pyrrolidin-3-yl]carbamate (495 mg, 1.83 mmol) in dry DCM (10 mL) was added 1M HCl in ether (10 mL) at 0 deg. C. The reaction was stirred at RT for 4 h and monitored by TLC. Concentration gave the desired amine (490 mg, 62%, white solid). ¹H NMR (400 MHz, DMSO-d6): δ 8.45 (s, Exchange with D20, 2H), 4.65-4.61 (m, 1H), 3.94-3.84 (m, 1H), 3.69-3.61 (m, 1H), 3.43-3.32 (m, 1H), 2.39-2.32 (m, 1H), 2.13-2.06 (m, 1H), 1.93-1.87 (m, 1H), 1.72-1.60 (m, 2H), 1.54-1.29 (m, 2H), 1.25-1.18 (m, 1H).

### Step 4

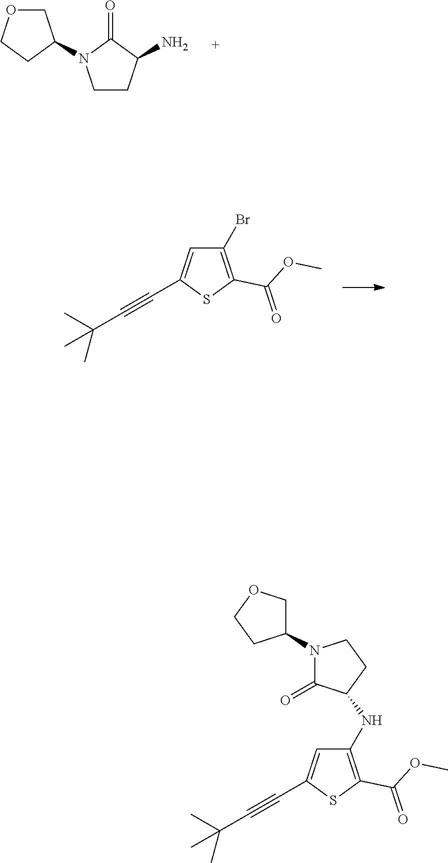 [SMILES: N[C@H]1CCN([C@H]2CCOC2)C1=O ; COC(=O)c1sc(C#CC(C)(C)C)cc1Br ; COC(=O)c1sc(C#CC(C)(C)C)cc1N[C@H]1CCN([C@H]2CCOC2)C1=O]

### Methyl 5-(3,3-dimethylbut-1-ynyl)-3-[[(3S)-2-oxo-1-[(3S)-tetrahydrofuran-3-yl]pyrrolidin-3-yl]amino]thiophene-2-carboxylate

A suspension of methyl-3-bromo-5-(3,3-dimethylbut-1-ynyl)thiophene-2-carboxylate (594 mg, 3.488 mmol), (3S)-3-amino-1-[(3S)-tetrahydrofuran-3-yl]pyrrolidin-2-one (700 mg, 2.32 mmol), Cs₂CO₃ (2.27 g, 6.98 mmol) in toluene (15 mL) was deoxygenated by purging a stream of argon for 60 min after which Pd(OAc)₂ (52.2 mg, 0.232 mmol) and (±)BINAP (144 mg, 0.232 mmol) were added and purging was continued for another 30 min and stirred at 100 deg. C. for 16 h. Reaction progress was monitored by TLC. After completion the reaction mixture was cooled to RT, diluted with EtOAc (100 mL) and filtered through celite. The filtrate was washed with water (2×50 mL), brine (20 mL), dried over Na₂SO₄, and concentrated. The resulting residue was purified by column chromatography (100-200 mesh silica gel, 60% EtOAc in pet ether as eluent) to afford the desired product (160 mg, 29%, yellow solid). TLC (50% EtOAc in pet ether) R_{f}=0.54. LC-MS conditions: column: HALO C18 (2.1×100 mm, 2.7 μM), mobile phases A: 5 mM NH₄OAc, B: acetonitrile; t/% B: 0 min/3%, 1.5/45, 2.5/45, 3.2/95, 4.7/95, 5/03; flow rate: 0.6 mL/min, sample diluent: MeOH, MS: m/z (obs.): 391.0 [M+H]⁺. Retention time: 3.44 min. ¹H NMR (400 MHz, DMSO-d6): δ 6.95 (s, 1H), 6.94 (d, J=6.8 Hz, exchanged with D₂O, 1H), 4.64-4.62 (m, 1H), 4.35-4.28 (m, 1H), 3.72 (s, 3H), 3.69-3.61 (m, 3H), 3.37-3.25 (m, 2H), 2.58-2.42 (m, 1H), 2.13-2.07 (m, 1H), 1.99-1.77 (m, 2H), 1.29 (s, 9H) 1.23-1.15 (m, 1H).

### Step 5

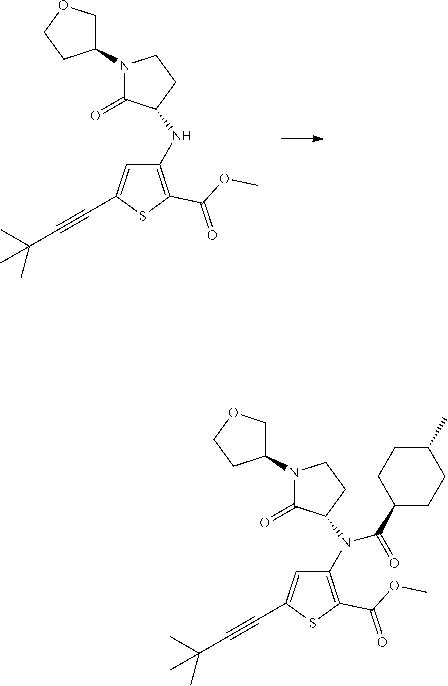 [SMILES: COC(=O)c1sc(C#CC(C)(C)C)cc1N[C@H]1CCN([C@H]2CCOC2)C1=O ; COC(=O)c1sc(C#CC(C)(C)C)cc1N(C(=O)[C@H]1CC[C@H](C)CC1)[C@H]1CCN([C@H]2CCOC2)C1=O]

### Methyl 5-(3,3-dimethylbut-1-ynyl)-3-[(trans-4-methylcyclohexanecarbonyl)-[(3S)-2-oxo-1-[(3S)-tetrahydrofuran-3-yl]pyrrolidin-3-yl]amino]thiophene-2-carboxylate

To a stirred solution of methyl 5-(3,3-dimethylbut-1-ynyl)-3-[[(3S)-2-oxo-1-[(3S)-tetrahydrofuran-3-yl]pyrrolidin-3-yl]amino]thiophene-2-carboxylate (165 mg, 0.423 mmol), pyridine (6 mL), DMAP (25.8 mg, 0.212 mmol) in dichloroethane (20 mL) at 0 deg. C., a stock solution of trans-4-methylcylohexanecarbonyl chloride (679 g, 4.23 mmol) in dichloroethane (5 mL) was added dropwise. After addition the reaction mixture was stirred at 100 deg. C. for 16 h. Reaction progress was monitored by TLC. After completion, the reaction mixture was diluted with EtOAc (150 mL), washed with aq. 2N HCl (40 mL), 10% NaHCO₃ solution (3×50 mL), water (30 mL), brine (20 mL), then dried over Na₂SO₄ and concentrated. The resulting residue was purified by column chromatography (100-200 mesh silica gel, 50% EtOAc in pet ether as eluent) to afford the desired product (118 mg, 54%, pale yellow solid). TLC (60% EtOAc in pet ether) R_{f}=0.51. LC-MS conditions: column HALO C18 (2.1×100 mm, 2.7 μM) mobile phases A: 5 mM NH₄OAc, B: acetonitrile; t/% B: 0 min/3%, 1.5/45, 2.5/45, 3.2/95, 4.7/95, 5/3, flow rate: 0.6 mL/min, sample diluent: MeOH. MS: m/z (obs.): 515.3 [M+H]. Retention time: 3.77 min.

### Step 6

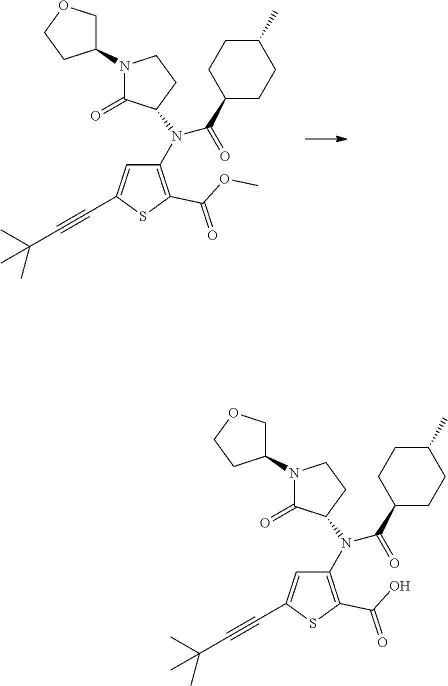

### 5-(3,3-Dimethylbut-1-ynyl)-3-[(trans-4-methylcyclohexanecaronyl)-[(3S)-2-oxo-1-[(3S)-tetrahydrofuran-3-yl]pyrrolidin-3-yl]amino]thiophene-2-carboxylic acid

To a stirred solution of methyl 5-(3,3-dimethylbut-1-ynyl)-3-[(trans-4-methylcyclohexanecarbonyl)-[(3S)-2-oxo-1-[(3S)-tetrahydrofuran-3-yl]pyrrolidin-3-yl]amino]thiophene-2-carboxylate (50 mg, 0.097 mmol) in a 1:1 mixture of THF and water (2 mL), LiOH*H₂O (12.3 mg, 0.291 mmol) was added at RT and stirred for 3 h. Reaction progress was monitored by TLC. The reaction mixture pH was adjusted to ˜1 using IM aq HCl, extracted with EtOAc (20 mL), washed with water (3×20 mL), brine (20 mL), dried over Na₂SO₄ and concentrated to afford 156 (35 mg, 66%, off-white solid). TLC (10% MeOH in CHCl₃) R_{f}=0.4. LC-MS conditions: column HALO C18 (2.1×100 mm, 2.7 μM), mobile phases A: 5 mM NH₄OAc, B: acetonitrile; t/% B: 0 min/3%, 1.5/45, 2.5/45, 3.2/95, 4.7/95, 5/3, flow rate 0.6 mL/min, sample diluent: MeOH. MS: m/z (obs.): 501.259 [M+H]⁺. Retention time: 1.87 min. ¹H NMR (400 MHz, DMSO-d6): δ 6.85 (s, 0.27H), 6.69 (s, 0.77H), 5.37-5.33 (m, 0.3H), 4.58-4.53 (m, 0.7H), 3.81-3.71 (m, 2H), 3.65-3.54 (m, 2H), 3.32-3.13 (m, 1H), 2.83-2.82 (m, 1H), 2.14-1.98 (m, 3H), 1.95-1.77 (m, 3H), 1.57-1.50 (m, 3H), 1.50-1.40 (m, 1H), 1.27 (s, 9H), 1.23-1.10.

### Preparation of Compound 157

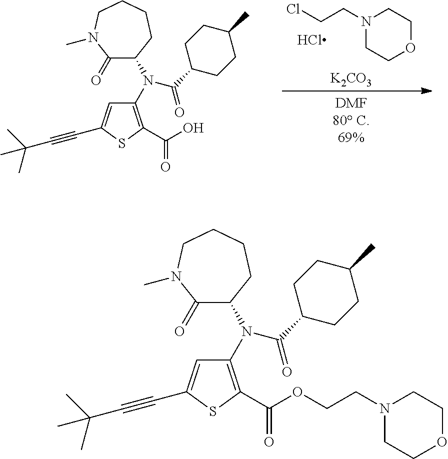 [SMILES: CN1CCCC[C@H](N(c2cc(C#CC(C)(C)C)sc2C(=O)O)C(=O)[C@H]2CC[C@H](C)CC2)C1=O ; "K2CO3" ; ClCCN1CCOCC1 ; CN1CCCC[C@H](N(c2cc(C#CC(C)(C)C)sc2C(=O)OCCN2CCOCC2)C(=O)[C@H]2CC[C@H](C)CC2)C1=O]

### Compound 157

A microwave vial was charged with starting acid (330 mg, 0.698 mmol), 4-(2-chloroethyl)morpholine (HCl salt) (195 mg, 1.05 mmol), potassium carbonate (290 mg, 2.10 mmol) and DMF (6 mL). The reaction was heated at 80° C. in microwave reactor for 30 min, after which time the reaction mixture was partitioned between EtOAc (100 mL) and water (100 mL). The organic layer was separated, washed with brine (70 mL), dried (magnesium sulfate), filtered and concentrated. The resulting residue was purified by MPLC using an Isco Combiflash (40 g column) 0-8% [2 M ammonia in MeOH] in DCM, linear gradient over 24 column volumes at 40 mL/min. Product obtained via evaporation of fractions is an oil. Oil was dissolved in MeCN (10 mL), treated with water (10 mL) then the resulting solution was frozen then lyophilized. Gave 157 (282 mg, 0.482 mmol, 69%) as a white solid. Analysis carried out by LCMS (10-90% aqueous MeCN, formic acid modifier, 5 min, C18) ESI-MS m/z calc. 585.32367. found 586.6 (M+1)+; Retention time: 2.35 minutes. 1H NMR (300.0 MHz, DMSO) δ 7.52 (s, 1H), 5.23 (d, J=11.5 Hz, 1H), 4.39-4.33 (m, 1H), 4.29-4.23 (m, 2H), 3.61 (d, J=3.9 Hz, 1H), 3.54 (t, J=4.5 Hz, 4H), 3.25 (dd, J=4.3, 15.0 Hz, 2H), 2.90 (s, 3H), 2.57 (t, J=5.2 Hz, 2H), 2.44-2.38 (m, 4H), 2.02-1.90 (m, 1H), 1.85-1.66 (m, H), 1.64-1.46 (m, 5H), 1.43-1.36 (m, 2H), 1.30 (s, 9H), 1.23-0.83 (m, 4H), 0.76 (d, J=6.5 Hz, 3H) and 0.70-0.48 (m, 2H) ppm.

### Preparation of Compound 158

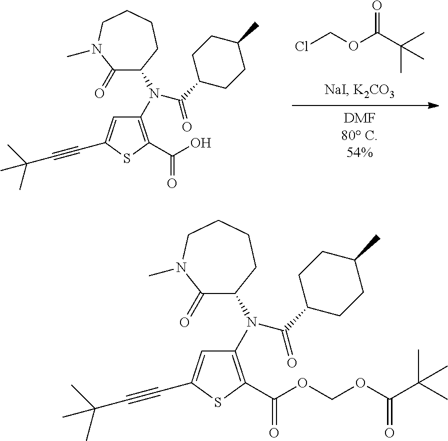

### Compound 158

A 20 mL microwave vial was charged with starting acid (369 mg, 0.7807 mmol), chloromethyl 2,2-dimethylpropanoate (141.1 mg, 135.0 μL, 0.9368 mmol), potassium carbonate (140.3 mg, 1.015 mmol), sodium iodide (95.66 mg, 0.6382 mmol) and DMF (5 mL). The reaction was heated at 80° C. in a microwave reactor for 30 min, after which time the reaction mixture was partitioned between EtOAc and water (80 mL each). The organic layer was separated, washed with brine (50 mL), dried (magnesium sulfate), filtered and concentrated. The resulting residue was purified by MPLC using an Isco Combiflash (40 g column) 0-50% EtOAc in heptane linear gradient over 24 column volumes at 30 mL/min. Product obtained via evaporation of fractions is an oil. Oil was dissolved in MeCN (10 mL), treated with water (10 mL) then the resulting solution was frozen then lyophilized. Gave 158 (250 mg, 0.421 mmol, 54%) as a white solid. Analysis carried out by LCMS (60-98% aqueous MeCN, formic acid modifier, 7 min, C4) ESI-MS m/z calc. 586.3077. found 587.62 (M+1)⁺; Retention time: 3.45 minutes. 1H NMR (300.0 MHz, DMSO) δ 7.55 (s, 1H), 5.91 (d, J=5.9 Hz, 1H), 5.79 (d, J=5.9 Hz, 1H), 5.21 (d, J=11.5 Hz, 1H), 3.59 (dd, J=11.2, 15.1 Hz, 1H), 3.24 (dd, 4.0, 15.0 Hz, 1H), 2.90 (s, 3H), 2.01-1.90 (m, 1H), 1.81-1.65 (m, 2H), 1.63-1.48 (m, 5H), 1.45-1.31 (m, 1H), 1.30 (s, 9H), 1.25-1.15 (m, 2H), 1.14 (s, 9H), 1.08-0.84 (m, 3H), 0.75 (d, J=6.4 Hz, 3H) and 0.70-0.45 (m, 2H) ppm.

### Preparation of Compound 159

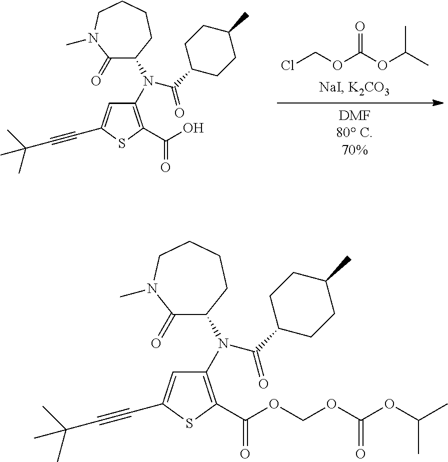

### Compound 159

A microwave vial was charged with starting acid (303 mg, 0.641 mmol), chloromethyl isopropyl carbonate (117.4 mg, 0.7693 mmol), potassium carbonate (115.2 mg, 0.8334 mmol), sodium iodide (46 mg, 0.31 mmol) and DMF (6 mL). Heated at 80° C. in microwave for 30 min, then the reaction mixture was partitioned between EtOAc and water (80 mL each). The organic layer was separated, washed with brine (50 mL), dried (magnesium sulfate) filtered and concentrated. The resulting residue was purified by MPLC using an Isco Combiflash (40 g column) 0-100% EtOAc in heptane linear gradient over 24 column volumes at 40 mL/min. Product obtained via evaporation of fractions is an oil. Oil was dissolved in MeCN (10 mL), treated with water (10 mL) then the resulting solution was frozen then lyophilized. Gave 159 (271 mg, 0.446 mmol, 70%) as a white solid. Analysis carried out by LCMS (60-98% aqueous MeCN, formic acid modifier, 7 min, C4) ESI-MS m/z calc. 588.2869. found 589.60 (M+1)+; Retention time: 3.06 minutes. 1H NMR (400.0 MHz, DMSO) δ 7.55 (s, 1H), 5.91 (d, J=6.2 Hz, 1H), 5.78 (d, J=6.2 Hz, 1H), 5.23 (d, J=11.8 Hz, 1H), 4.81 (qn, J=6.2 Hz, 1H), 3.61 (dd, J=11.4, 15.1 Hz, 1H), 3.29-3.22 (m, 1H), 2.90 (s, 3H), 1.94 (t, J=11.6 Hz, 1H), 1.79-1.33 (m, 10H), 1.30 (s, 9H), 1.24 (dd, J=1.5, 6.2 Hz, 6H), 1.20-0.90 (m, 3H), 0.76 (d, J=6.4 Hz, 3H) and 0.73-0.48 (m, 2H) ppm.

### Preparation of Compound 160

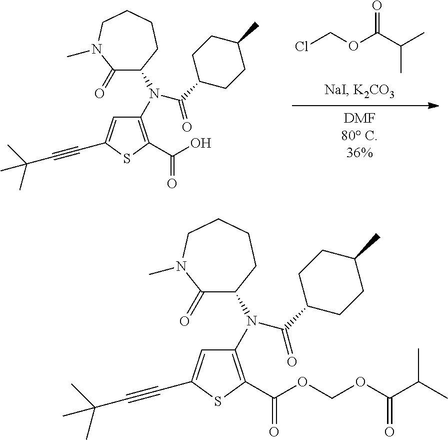

### Compound 160

A 20 mL microwave vial was charged with starting acid (375 mg, 0.793 mmol), chloromethyl 2-methylpropanoate (250 mg, 1.83 mmol), potassium carbonate (142.5 mg, 1.031 mmol), sodium iodide (97.2 mg, 26.5 μL, 0.649 mmol) and DMF (6 mL). Microwaved at 80° C. for 25 min, after which time the reaction mixture was partitioned between EtOAc and water (80 mL each). The organic layer was separated, washed with brine (50 mL), dried (magnesium sulfate), filtered and concentrated. The resulting residue was purified by MPLC using an Isco Combiflash (40 g column) 0-100% EtOAc in heptane linear gradient over 24 column volumes at 40 mL/min. Product obtained via evaporation of fractions is an oil. Oil was dissolved in MeCN (5 mL), treated with water (5 mL) then the resulting solution was frozen then lyophilized. Gave 160 (171 mg, 0.288 mmol, 36%) as a white solid. Analysis carried out by LCMS (60-98% aqueous MeCN, formic acid modifier, 7 min, C4) ESI-MS m/z calc. 572.292. found 573.61 (M+1)+; Retention time: 3.18 minutes. ¹H NMR (300.0 MHz, DMSO) δ 7.55 (s, 1H), 5.91 (d, J=6.0 Hz, 1H), 5.78 (d, J=6.0 Hz, 1H), 5.21 (d, J=11.5 Hz, 2H), 3.57 (d, J=11.1 Hz, 1H), 3.27 (d, J=2.9 Hz, 1H), 2.90 (s, 3H), 2.60-2.50 (m, 1H), 2.01-1.87 (m, 1H), 1.82-1.33 (m, 9H), 1.30 (s, 9H), 1.29-1.11 (m, 2H), 1.09 (dd, J=1.2, 7.0 Hz, 6H), 1.03-0.86 (m, 1H), 0.76 (d, J=6.4 Hz, 3H) and 0.71-0.47 (m, 2H) ppm.

### Preparation of Compound 161

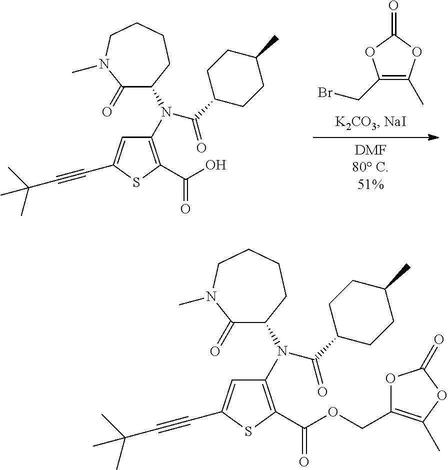

### Compound 161

A 20 mL microwave vial was charged with starting acid (320 mg, 0.677 mmol), 4-(bromomethyl)-5-methyl-1,3-dioxol-2-one (156.8 mg, 0.8124 mmol), potassium carbonate (121.6 mg, 0.8801 mmol), sodium iodide (48.6 mg, 13.2 μL, 0.324 mmol) and DMF (6 mL). Heated at 80° C. in microwave for 25 min, then the reaction mixture was partitioned between EtOAc and water (80 mL each). The organic layer was separated, washed with brine (50 mL), dried (magnesium sulfate), filtered and concentrated. The resulting residue was purified by MPLC using an Isco Combiflash (40 g column) 0-100% EtOAc in hexane linear gradient over 24 column volumes at 40 mL/min. Product obtained via evaporation of fractions was dissolved in MeCN (10 mL), treated with water (10 mL) then the resulting solution was frozen then lyophilized. Gave 161 (210 mg, 0.347 mmol, 51%) as a white solid. Analysis carried out by LCMS (60-98% aqueous MeCN, formic acid modifier, 7 min, C4) ESI-MS m/z calc. 584.2556. found 585.57 (M+1)+; Retention time: 2.61 minutes. ¹H NMR (300.0 MHz, DMSO) δ 7.52 (s, 1H), 5.28-5.08 (m, 3H), 3.61 (dd, J=11.3, 15.1, 1H), 3.29-3.22 (m, 1H), 2.90 (s, 3H), 2.17 (s, 3H), 2.00-1.31 (m, 9H), 1.30 (s, 9H), 1.28-0.85 (m, 5H), 0.75 (d, J=6.5 Hz, 3H) and 0.70-0.46 (m, 2H) ppm.

### Preparation of Compound 162

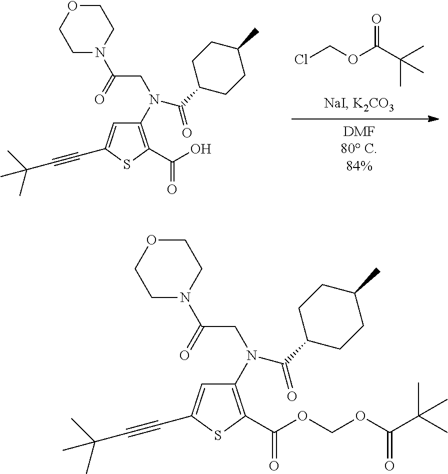

### Compound 162

A 20 mL microwave vial was charged with starting acid (300 mg, 0.6321 mmol), potassium carbonate (113.6 mg, 0.8217 mmol), sodium iodide (66.33 mg, 18.09 μL, 0.4425 mmol), DMF (10 mL) and chloromethyl 2,2-dimethylpropanoate (114.2 mg, 109.3 μL, 0.7585 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-60% EtOAc in hexanes linear gradient over 24 column volumes at 40 mL/min. Product isolated from column was dissolved in MeCN (10 mL), treated with water (8 mL) then the resulting mixture was frozen and lyophilized. Gave 162 (316 mg, 0.534 mmol, 84%) as a white solid. Analysis carried out by LCMS (60-98% aqueous MeCN, formic acid modifier, 7 min, C4) ESI-MS m/z calc. 588.2869. found 589.6 (M+1)+; Retention time: 3.04 minutes. ¹H NMR (300.0 MHz, DMSO) δ 7.27 (s, 1H), 5.89 (d, J=5.9 Hz, 1H), 5.82 (d, J=5.9 Hz, 1H), 4.92 (d, J=16.7 Hz, 1H), 3.85 (d, J=16.6 Hz, 1H), 3.55 (br s, 4H), 3.41-3.35 (m, 4H), 2.11-1.98 (m, 1H), 1.70-1.34 (m, 6H), 1.30 (s, 9H), 1.28-1.20 (m, 1H), 1.14 (s, 9H), 0.77 (d, J=6.5 Hz, 3H) and 0.74-0.57 (m, 2H) ppm.

### Preparation of Compound 163

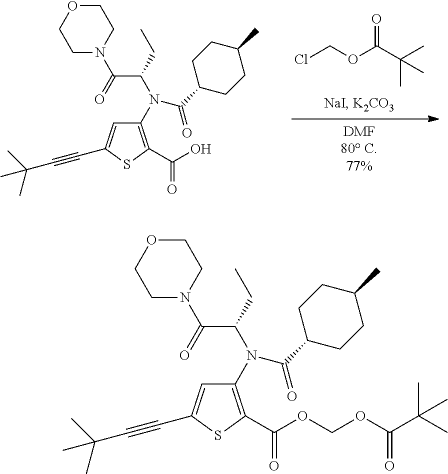

### Compound 163

A 20 mL microwave vial was charged with starting acid (300 mg, 0.597 mmol), potassium carbonate (107 mg, 0.776 mmol), sodium iodide (62.6 mg, 17.1 μL, 0.418 mmol), DMF (10 mL) and chloromethyl 2,2-dimethylpropanoate (108 mg, 103 μL, 0.716 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-60% EtOAc in hexanes linear gradient over 24 column volumes at 40 mL/min. Product isolated from column was dissolved in MeCN (10 mL), treated with water (8 mL) then the resulting mixture was frozen and lyophilized. Gave 163 (301 mg, 0.470 mmol, 79%) as a white solid. Analysis carried out by LCMS (60-98% aqueous MeCN, formic acid modifier, 7 min, C4) ESI-MS m/z calc. 616.31824. found 617.56 (M+1)+; Retention time: 3.49 minutes. (1H NMR shows rotamers) 1H NMR (300.0 MHz, DMSO) δ 7.34 (s, 0.6H), 7.27 (s, 0.4H), 5.91-5.75 (m, 2H), 5.56 (t, J=7.5 Hz, 0.4H), 5.19 (dd, J=6.4, 8.6 Hz, 0.6H), 3.77-3.39 (m, 7.6H), 3.21-3.10 (m, 0.4H), 2.00-1.82 (m, 1H), 1.65-1.35 (m, 6H), 1.34-1.16 (m, 12H), 1.13 (s, 9H) and 0.80-0.68 (m, 8H) ppm.

### Preparation of Compound 164

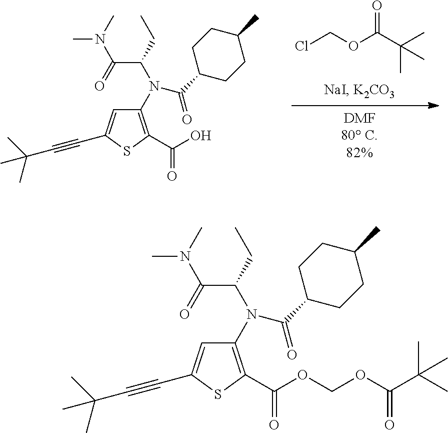

### Compound 164

A 20 mL microwave vial was charged with starting acid (300 mg, 0.651 mmol), potassium carbonate (117 mg, 0.847 mmol), sodium iodide (68.3 mg, 18.6 μL, 0.456 mmol), DMF (10 mL) and chloromethyl 2,2-dimethylpropanoate (118 mg, 113 μL, 0.782 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-60% EtOAc in hexanes linear gradient over 24 column volumes at 40 mL/min. Product isolated from column was dissolved in MeCN (10 mL), treated with water (8 mL) then the resulting mixture was frozen and lyophilized. Gave 165 (308 mg, 0.532 mmol, 82%) as a white solid. Analysis carried out by LCMS (60-98% aqueous MeCN, formic acid modifier, 7 min, C4) ESI-MS m/z calc. 574.3077. found 575.59 (M+1)+; Retention time: 3.53 minutes. (1H NMR shows rotamers) 1H NMR (300.0 MHz, DMSO) δ 7.31 (s, 0.6H), 7.24 (s, 0.4H), 5.90 (d, J=6.0 Hz, 0.6H), 5.81 (d, J=5.0 Hz, 1.4H), 5.58 (t, J=7.5 Hz, 0.4H), 5.16 (dd, J=6.2, 8.8 Hz, 0.6H), 3.10 (s, 1.8H), 3.05 (s, 1.2H), 2.85 (s, 1.8H), 2.65 (s, 1.2H), 1.99-1.84 (m, 1H), 1.66-1.33 (m, 6H), 1.33-1.15 (m, 12H), 1.14 (s, 9H) and 0.79-0.49 (m, 8H) ppm.

### Preparation of Compound 165

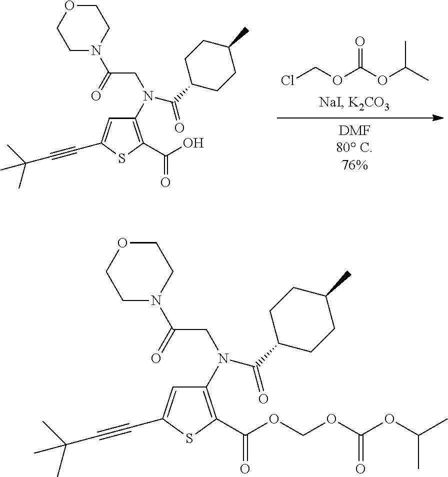

### Compound 165

A 20 mL microwave vial was charged with starting acid (300 mg, 0.632 mmol), potassium carbonate (114 mg, 0.822 mmol), sodium iodide (66.3 mg, 18.1 μL, 0.442 mmol), DMF (10 mL) and chloromethyl isopropyl carbonate (116 mg, 0.758 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-80% EtOAc in hexanes linear gradient over 24 column volumes at 40 mL/min. Product isolated from column was dissolved in MeCN (10 mL), treated with water (8 mL) then the resulting mixture was frozen and lyophilized. Gave 166 (291 mg, 0.479 mmol, 76%) as a white solid. Analysis carried out by LCMS (60-98% aqueous MeCN, formic acid modifier, 7 min, C4) ESI-MS m/z calc. 590.2662. found 591.59 (M+1)+; Retention time: 2.59 minutes. 1H NMR (300.0 MHz, DMSO) δ 7.27 (s, 1H), 5.84 (dd, J=6.2, 9.7 Hz, 2H), 4.91 (d, J=16.7 Hz, 1H), 4.80 (septet, J=6.2 Hz, 1H), 3.89 (d, J=16.7 Hz, 1H), 3.55 (br s, 4H), 3.45-3.35 (m, 4H), 2.13-1.98 (m, 1H), 1.79-1.33 (m, 7H), 1.30 (s, 9H), 1.24 (d, J=6.2 Hz, 6H), 0.77 (d, J=6.4 Hz, 3H) and 0.75-0.57 (m, 2H) ppm.

### Preparation of Compound 166

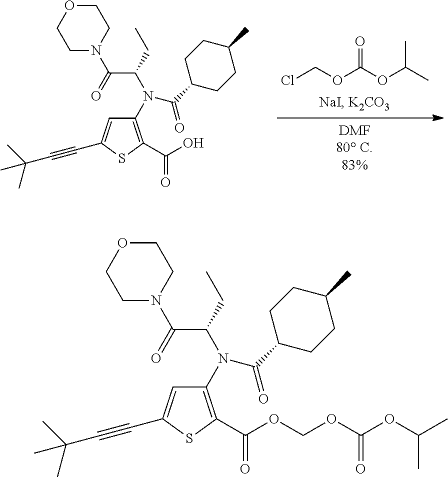

### Compound 166

A 20 mL microwave vial was charged with starting acid (71.49 mg, 0.5173 mmol), sodium iodide (41.75 mg, 11.39 μL, 0.2785 mmol), DMF (7 mL) and chloromethyl isopropyl carbonate (72.86 mg, 0.4775 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-80% EtOAc in hexanes linear gradient over 24 column volumes at 40 mL/min. Product isolated from column was dissolved in MeCN (10 mL), treated with water (8 mL) then the resulting mixture was frozen and lyophilized. Gave 166 (213 mg, 0.331 mmol, 83%) as a white solid. Analysis carried out by LCMS (60-98% aqueous MeCN, formic acid modifier, 7 min, C4) ESI-MS m/z calc. 618.2975. found 619.61 (M+1)+; Retention time: 3.02 minutes. (1H NMR shows rotamers) 1H NMR (300.0 MHz, DMSO) δ 7.36 (s, 0.6H), 7.27 (s, 0.4H), 5.89 (d, J=6.3 Hz, 0.6H), 5.81-5.74 (m, 1.4H), 5.56 (t, J=7.4 Hz, 0.4H), 5.19 (dd, J=6.5, 8.5 Hz, 0.6H), 4.88-4.73 (m, 2H), 3.73-3.35 (m, 7.6H), 3.23-3.12 (m, 0.4H), 1.97-1.84 (m, 1H), 1.66-1.34 (m, 6H), 1.32-1.07 (m, 17H) and 0.81-0.47 (m, 8H) ppm.

### Preparation of Compound 167

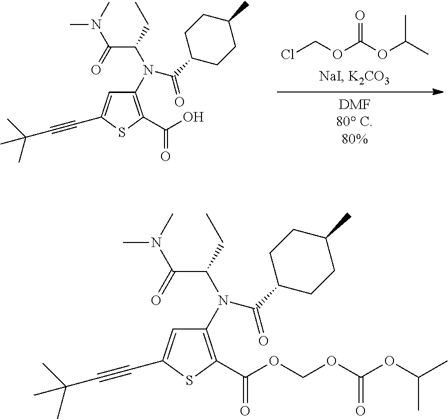

### Compound 167

A 20 mL microwave vial was charged with starting acid (300 mg, 0.651 mmol), potassium carbonate (117 mg, 0.847 mmol), sodium iodide (68.3 mg, 18.6 μL, 0.456 mmol), DMF (10 mL) and chloromethyl isopropyl carbonate (119 mg, 0.782 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-80% EtOAc in hexanes linear gradient over 24 column volumes at 40 mL/min. Product isolated from column was dissolved in MeCN (10 mL), treated with water (8 mL) then the resulting mixture was frozen and lyophilized. Gave 167 (306 mg, 0.519 mmol, 80%) as a white solid. Analysis carried out by LCMS (60-98% aqueous MeCN, formic acid modifier, 7 min, C4) ESI-MS m/z calc. 576.2869. found 577.57 (M+1)+; Retention time: 3.05 minutes. (¹H NMR shows rotamers) ¹H NMR (300.0 MHz, DMSO) δ 7.33 (s, 0.55H), 7.24 (s, 0.45H), 5.89 (d, J=6.3 Hz, 0.55H), 5.81 (dd, J=6.3, 8.0 Hz, 1H), 5.73 (d, J=6.3 Hz, 0.45H), 5.58 (t, J=7.5 Hz, 0.45H), 5.16 (d, J=2.4 Hz, 0.55H), 4.87-4.85 (m, 1H), 3.11 (s, 1.65H), 3.02 (s, 1.35H), 2.85 (s, 1.65H), 2.63 (s, 1.35H), 1.99-1.86 (m, 1H), 1.67-1.35 (m, 6H), 1.34-1.08 (m, 18H) and 0.82-0.51 (m, 8H) ppm.

### Preparation of Compound 168

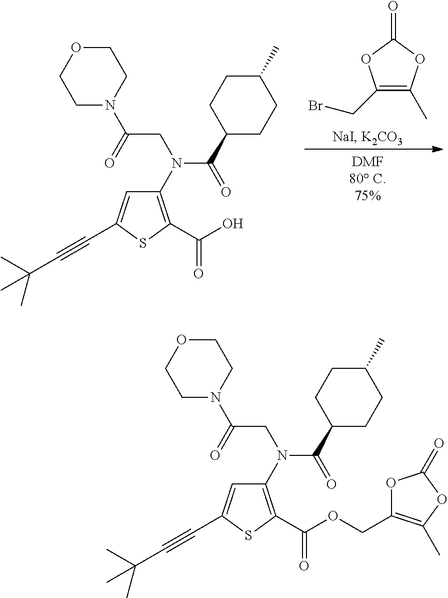

### Compound 168

A 20 mL microwave vial was charged with starting acid (300 mg, 0.632 mmol), potassium carbonate (114 mg, 0.822 mmol), sodium iodide (66.3 mg, 18.1 μL, 0.442 mmol), DMF (10 mL) and 4-(bromomethyl)-5-methyl-1,3-dioxol-2-one (146 mg, 0.758 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-100% EtOAc in hexanes linear gradient over 24 column volumes at 40 mL/min. Product isolated from column was dissolved in MeCN (10 mL), treated with water (8 mL) then the resulting mixture was frozen and lyophilized. Gave 168 (290 mg, 0.476 mmol, 75%) as a white solid. Analysis carried out by LCMS (60-98% aqueous MeCN, formic acid modifier, 7 min, C4) ESI-MS m/z calc. 586.23486. found 587.55 (M+1)+; Retention time: 2.25 minutes. ¹H NMR (300.0 MHz, DMSO) δ 7.24 (s, 1H), 5.18 (d, J=14.1 Hz, 1H), 5.12 (d, J=14.1 Hz, 1H), 4.89 (d, J=16.7 Hz, 1H), 3.92 (d, J=16.7 Hz, 1H), 3.61-3.48 (m, 4H), 3.47-3.33 (m, 4H), 2.17 (s, 3H), 2.13-2.00 (m, 1H), 1.73-1.33 (m, 5H), 1.30 (s, 9H), 1.30-1.15 (m, 2H), 0.77 (d, J=6.5 Hz, 3H) and 0.73-0.55 (m, 2H) ppm.

### Preparation of Compound 169

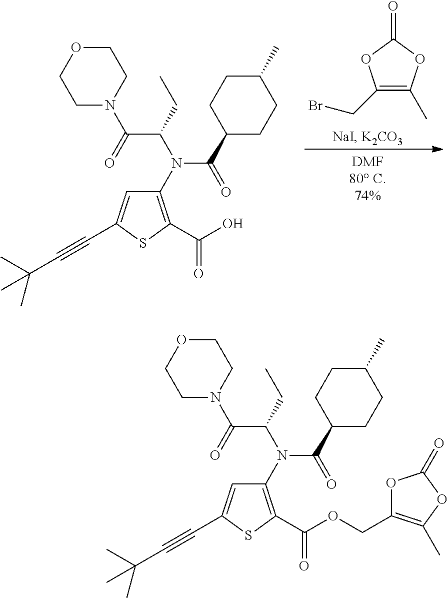

### Compound 169

A 20 mL microwave vial was charged with starting acid (300 mg, 0.597 mmol), potassium carbonate (107 mg, 0.776 mmol), sodium iodide (62.6 mg, 17.1 μL, 0.418 mmol), DMF (10 mL) and 4-(bromomethyl)-5-methyl-1,3-dioxol-2-one (138 mg, 0.716 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-100% EtOAc in hexanes linear gradient over 24 column volumes at 40 mL/min. Product isolated from column was dissolved in MeCN (10 mL), treated with water (8 mL) then the resulting mixture was frozen and lyophilized. Gave 169 (280 mg, 0.441 mmol, 74%) as a white solid. Analysis carried out by LCMS (60-98% aqueous MeCN, formic acid modifier, 7 min, C4) ESI-MS m/z calc. 614.2662. found 615.58 (M+1)+; Retention time: 2.61 minutes. 1H NMR shows rotamers. 1H NMR (300.0 MHz, DMSO) δ 7.31 (s, 0.6H), 7.24 (s, 0.4H), 5.55 (t, J=7.5 Hz, 0.4H), 5.25-5.05 (m, 2.6H), 3.78-3.40 (m, 7.6H), 3.09-2.99 (m, 0.4H), 2.17 (s, 3H), 1.98-1.85 (m, 1H), 1.65-1.33 (m, 6H), 1.32-1.05 (m, 12H) and 0.81-0.47 (m, 8H) ppm.

### Preparation of Compound 170

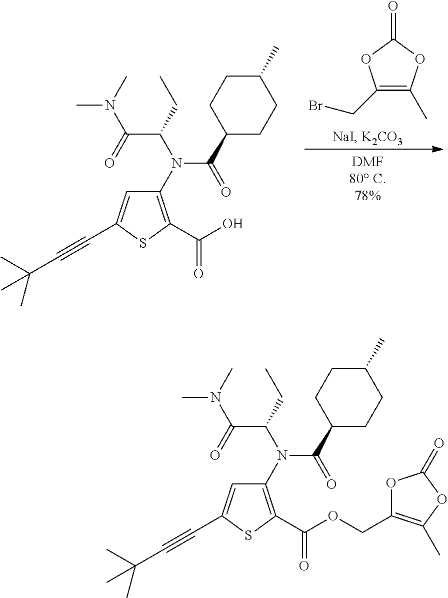

### Compound 170

A 20 mL microwave vial was charged with starting acid (300 mg, 0.651 mmol), potassium carbonate (117 mg, 0.847 mmol), sodium iodide (68.3 mg, 18.6 μL, 0.456 mmol), DMF (10 mL) and 4-(bromomethyl)-5-methyl-1,3-dioxol-2-on (151 mg, 0.782 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-100% EtOAc in hexanes linear gradient over 24 column volumes at 40 mL/min. Product isolated from column was dissolved in MeCN (10 mL), treated with water (8 mL) then the resulting mixture was frozen and lyophilized. Gave 170 (290 mg, 0.506 mmol, 78%) as a white solid. Analysis carried out by LCMS (60-98% aqueous MeCN, formic acid modifier, 7 min, C4) ESI-MS m/z calc. 572.2556. found 573.61 (M+1)+; Retention time: 2.66 minutes. 1H NMR shows rotamers. 1H NMR (300.0 MHz, DMSO) δ 7.28 (s, 0.6H), 7.21 (s, 0.4H), 5.55 (t, J=7.5 Hz, 0.4H), 5.24-5.12 (m, 2.2H), 5.02 (d, J=14.2 Hz, 0.4H), 3.10 (s, 1.8H), 3.05 (s, 1.2H), 2.85 (s, 1.8H), 2.64 (s, 1.2H), 2.17 (s, 3H), 1.96-1.85 (m, 1H), 1.66-1.33 (m, 6H), 1.32-1.05 (m, 12H) and 0.79-0.47 (m, 8H) ppm.

### Preparation of Compound 171

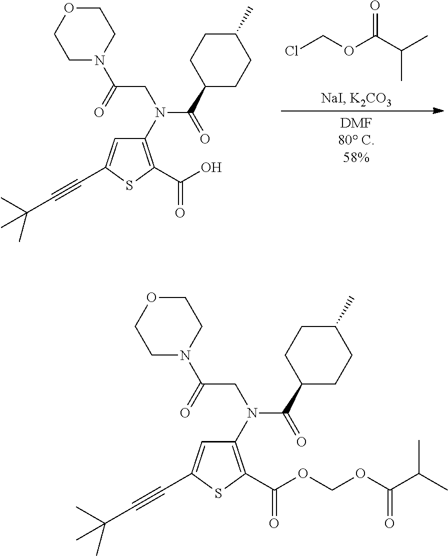

### Compound 171

A 20 mL microwave vial was charged with starting acid (300 mg, 0.632 mmol), potassium carbonate (114 mg, 0.822 mmol), sodium iodide (66.3 mg, 181 μL, 0.442 mmol), DMF (10 mL) and chloromethyl 2-methylpropanoate (104 mg, 0.758 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-80% EtOAc in hexanes linear gradient over 24 column volumes at 40 mL/min. Product isolated from column was dissolved in MeCN (10 mL), treated with water (8 mL) then the resulting mixture was frozen and lyophilized. Gave 171 (214 mg, 0.368 mmol, 58%) as a white solid. Analysis carried out by LCMS (60-98% aqueous MeCN, formic acid modifier, 7 min, C4) ESI-MS m/z calc. 574.2713. found 575.59 (M+1)+; Retention time: 2.9 minutes. 1H NMR (300.0 MHz, DMSO) δ 7.26 (s, 1H), 5.85 (dd, J=5.9, 16.9 Hz, 2H), 4.91 (d, J=16.7 Hz, 1H), 3.86 (d, J=16.7 Hz, 1H), 3.62-3.48 (m, 4H), 3.44-3.33 (m, 4H), 2.65-2.55 (m, 1H), 2.13-1.98 (m, 1H), 1.78-1.35 (m, 5H), 1.30 (s, 9H), 1.29-1.18 (m, 2H), 1.08 (d, J=7.0 Hz, 6H), 0.77 (d, J=6.5 Hz, 3H) and 0.74-0.58 (m, 2H) ppm.

### Preparation of Compound 172

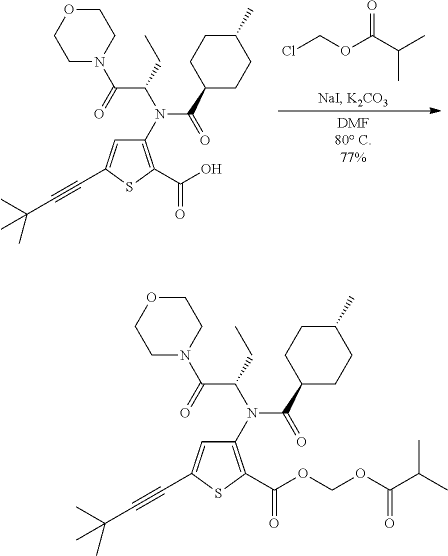

### Compound 172

A 20 mL microwave vial was charged with starting acid (300 mg, 0.597 mmol), potassium carbonate (107 mg, 0.776 mmol), sodium iodide (62.6 mg, 17.1 μL, 0.418 mmol), DMF (10 mL) and chloromethyl 2-methylpropanoate (97.8 mg, 0.716 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-80% EtOAc in hexanes linear gradient over 24 column volumes at 40 mL/min. Product isolated from column was dissolved in MeCN (10 mL), treated with water (8 mL) then the resulting mixture was frozen and lyophilized. Gave 172 (280 mg, 0.460 mmol, 77%) as a white solid. Analysis carried out by LCMS (60-98% aqueous MeCN, formic acid modifier, 7 min, C4) ESI-MS m/z calc. 602.30255. found 603.68 (M+1)+; Retention time: 3.36 minutes. 1H NMR shows rotamers. 1H NMR (300.0 MHz, DMSO) δ 7.34 (s, 0.6H), 7.27 (s, 0.4H), 5.90 (d, J=6.0 Hz, 0.6H), 5.85-5.76 (m, 1.4H), 5.56 (t, J=7.3 Hz, 0.4H), 5.18 (dd, J=6.4, 8.6 Hz, 0.6H), 3.75-3.35 (m, 7.6H), 3.20-3.09 (m, 0.4H), 2.62-2.53 (m, 1H), 1.98-1.82 (m, 1H), 1.65-1.34 (m, 6H), 1.32-1.13 (m, 12H), 1.10-1.06 (m, 6H) and 0.82-0.49 (m, 8H) ppm.

### Preparation of Compound 173

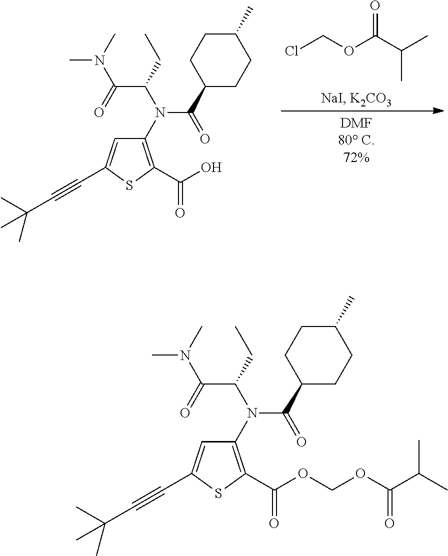

### Compound 173

A 20 mL microwave vial was charged with starting acid (300 mg, 0.651 mmol), potassium carbonate (117 mg, 0.847 mmol), sodium iodide (68.3 mg, 18.6 μL, 0.456 mmol), DMF (10 mL) and chloromethyl 2-methylpropanoate (107 mg, 0.782 mmol). Heated at 80° C. in microwave for 20 min, then the reaction mixture was partitioned between EtOAc and water (80 mL each). The organic layer was separated, washed with water then brine (80 mL each), dried (magnesium sulfate), filtered and concentrated. The resulting residue was purified by MPLC using an Isco Combiflash (40 g column) 0-80% EtOAc in hexanes linear gradient over 24 column volumes at 40 mL/min. Product isolated from column was dissolved in MeCN (10 mL), treated with water (8 mL) then the resulting mixture was frozen and lyophilized. Gave 173 (265 mg, 0.466 mmol, 72%) as a white solid. Analysis carried out by LCMS (60-98% aqueous MeCN, formic acid modifier, 7 min, C4) ESI-MS m/z calc. 560.292. found 561.64 (M+1)+; Retention time: 3.41 minutes. 1H NMR shows rotamers. 1H NMR (300.0 MHz, DMSO) δ 7.31 (s, 0.6H), 7.23 (s, 0.4H), 5.90 (d, J=6.0 Hz, 0.6H), 5.82-5.75 (m, 1.4H), 5.58 (t, J=7.5 Hz, 0.4H), 5.15 (dd, J=6.2, 8.8 Hz, 0.6H), 3.10 (s, 1.8H), 3.04 (s, 1.2H), 2.85 (s, 1.8H), 2.65 (s, 1.2H), 2.63-2.53 (m, 1H), 1.99-1.85 (m, 1H), 1.65-1.32 (m, 6H), 1.32-1.12 (m, 12H), 1.08 (d, J=7.0 Hz, 6H) and 0.79-0.47 (m, 8H) ppm.

### Preparation of Compound 174 (Prophetic)

### Step 1

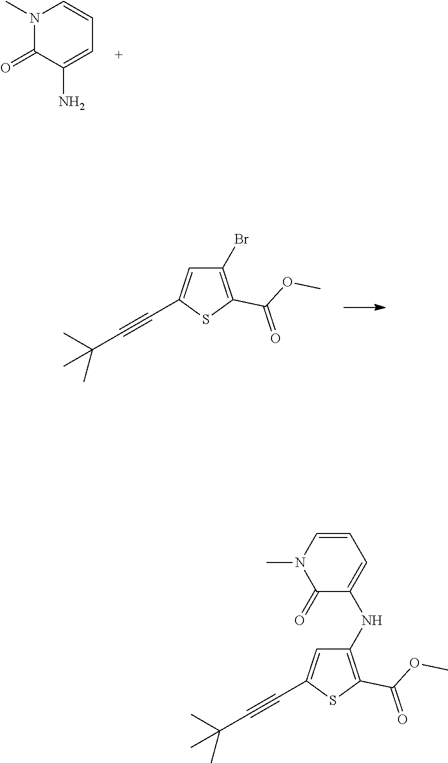

### Methyl 5-(3,3-dimethylbut-1-ynyl)-3-(1-methyl-2-oxo-1,2-dihydropyridin-3-ylamino)thiophene-2-carboxylate

A suspension of methyl-3-bromo-5-(3,3-dimethylbut-1-ynyl)thiophene-2-carboxylate (594 mg, 3.49 mmol), 3-amino-1-methylpyridin-2(1H)-one (288 mg, 2.32 mmol), Cs₂CO₃ (2.27 g, 6.98 mmol) in toluene (15 mL) is deoxygenated by purging a stream of argon for 60 min after which Pd(OAc)₂ (52.2 mg, 0.232 mmol) and (±)BINAP (144 mg, 0.232 mmol) is added and purging continued for another 30 min and stirred at 100 deg. C. for 16 h. Reaction progress is monitored by TLC. After completion the reaction mixture is cooled to RT, diluted with EtOAc (100 mL) and filtered through celite. The filtrate is washed with water (2×50 mL), brine (20 mL), dried over Na₂SO₄, and concentrated. The resulting residue is purified by column chromatography (100-200 mesh silica gel, 60% EtOAc in pet ether as eluent) to afford the desired product.

### Step 2

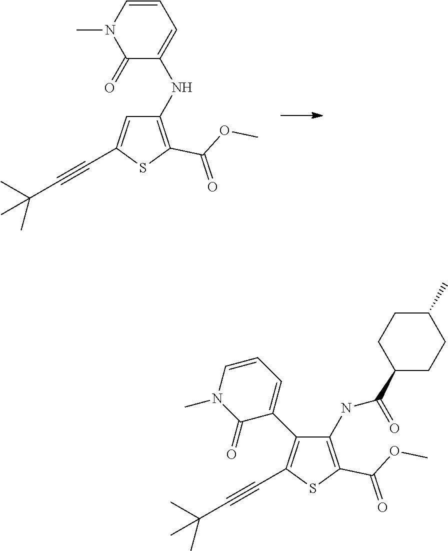 [SMILES: COC(=O)c1sc(C#CC(C)(C)C)cc1Nc1cccn(C)c1=O ; COC(=O)c1sc(C#CC(C)(C)C)c(-c2cccn(C)c2=O)c1NC(=O)[C@H]1CC[C@H](C)CC1]

### Compound 174, methyl ester

To a stirred solution of methyl 5-(3,3-dimethylbut-1-ynyl)-3-(1-methyl-2-oxo-1,2-dihydropyridin-3-ylamino)thiophene-2-carboxylate (146 mg, 0.423 mmol), pyridine (6 mL), DMAP (25.8 mg, 0.212 mmol) in dichloroethane (20 mL) at 0 deg. C., a stock solution of trans-4-methylcylohexanecarbonyl chloride (679 g, 4.23 mmol) in dichloroethane (5 mL) is added dropwise. After addition the reaction mixture is stirred at 100 deg. C. for 16 h. Reaction progress is monitored by TLC. After completion, the reaction mixture is diluted with EtOAc (150 mL), washed with aq. 2N HCl (40 mL), 10% NaHCO₃ solution (3×50 mL), water (30 mL), brine (20 mL), then dried over Na₂SO₄ and concentrated. The resulting residue is purified by column chromatography (100-200 mesh silica gel, 50% EtOAc in pet ether as eluent) to afford the desired product.

### Step 3

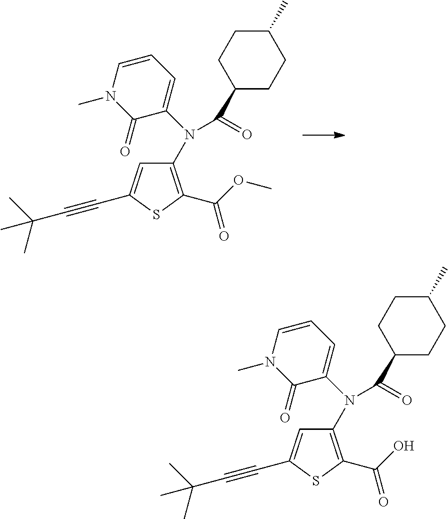

### Compound 174

To a stirred solution of methyl ester (50 mg, 0.11 mmol) in a 1:1 mixture of THF and water (2 mL), LiOH*H₂O (12.3 mg, 0.291 mmol) is added at RT and stirred for 3 h. Reaction progress is monitored by TLC. The reaction mixture pH is adjusted to −1 using IM aq HCl, and the mixture extracted with EtOAc (20 mL), washed with water (3×20 mL), brine (20 mL), dried over Na₂SO₄ and concentrated to afford 174.

### Preparation of Compound 175 (Prophetic)

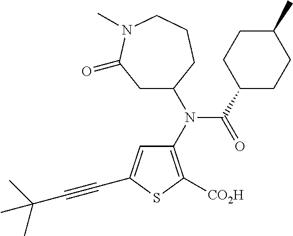 [SMILES: CN1CCCC(N(c2cc(C#CC(C)(C)C)sc2C(=O)O)C(=O)[C@H]2CC[C@H](C)CC2)CC1=O]

Compound 175 can be prepared according to the procedures described in compound 107, 112.

### Preparation of Compound 176 (Prophetic)

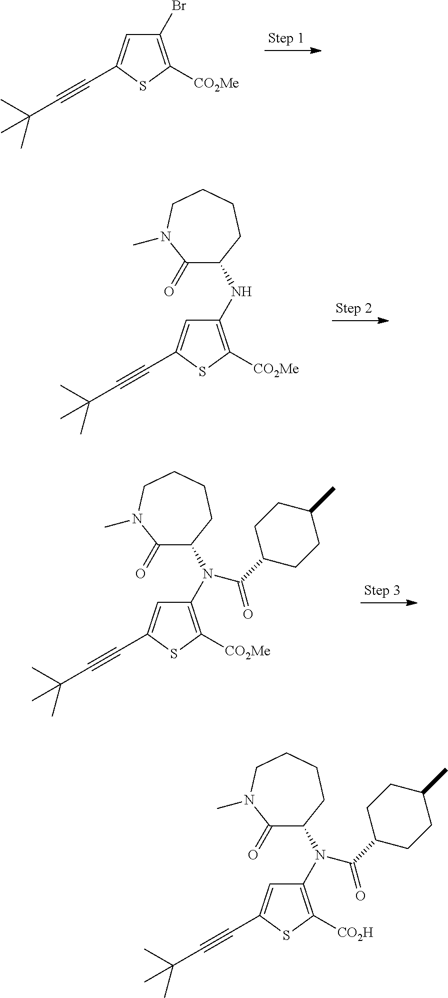

Compound 176 can be prepared according to the procedures described in compounds 95, 128, 144.

### Preparation of Compound 177 (Prophetic)

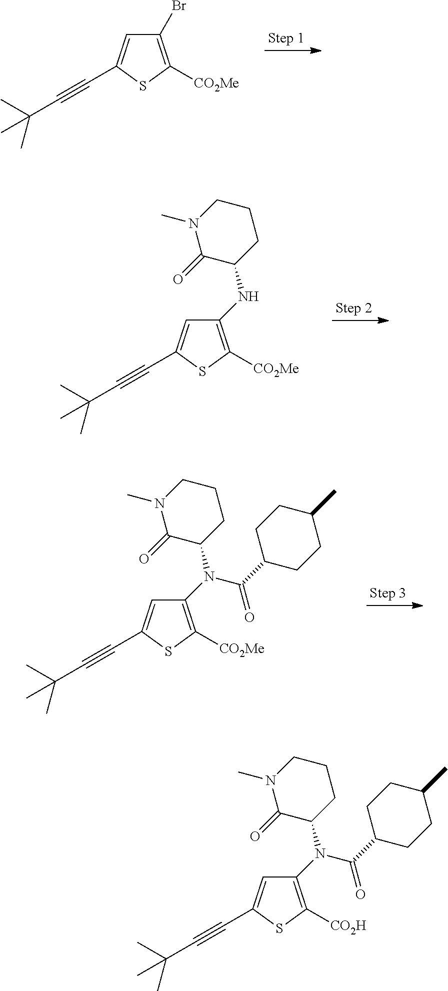

Compound 177 can be prepared according to the procedures described in compounds 117, 128, 144.

### Preparation of Compound 178 (Prophetic)

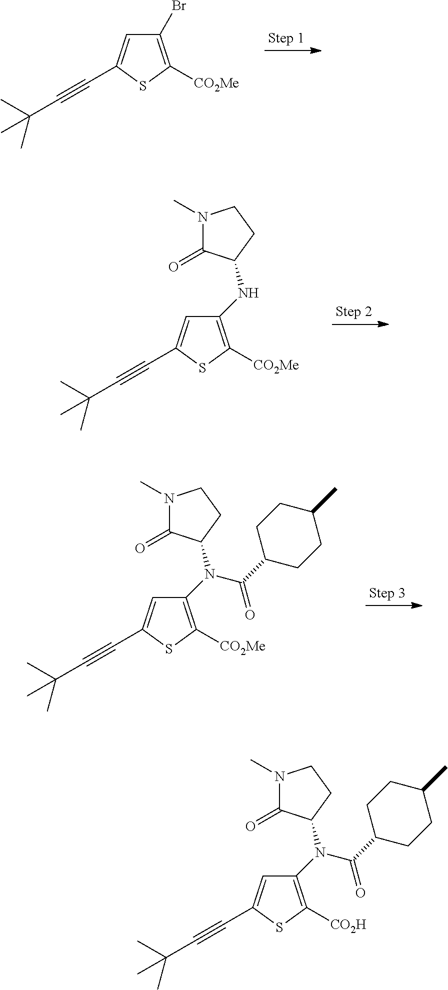

Compound 178 can be prepared according to the procedures described in compounds 83, 128, 144.

### Preparation of Compound 179 (Prophetic)

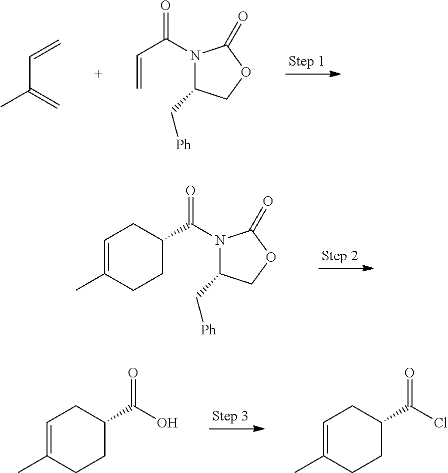

Steps 1, 2:

Steps 1 and 2 can be performed as described in J. Am. Chem. Soc. 1988, 110(4), 1238.

Step 3:

Step 3 can be performed as described for compound 6.

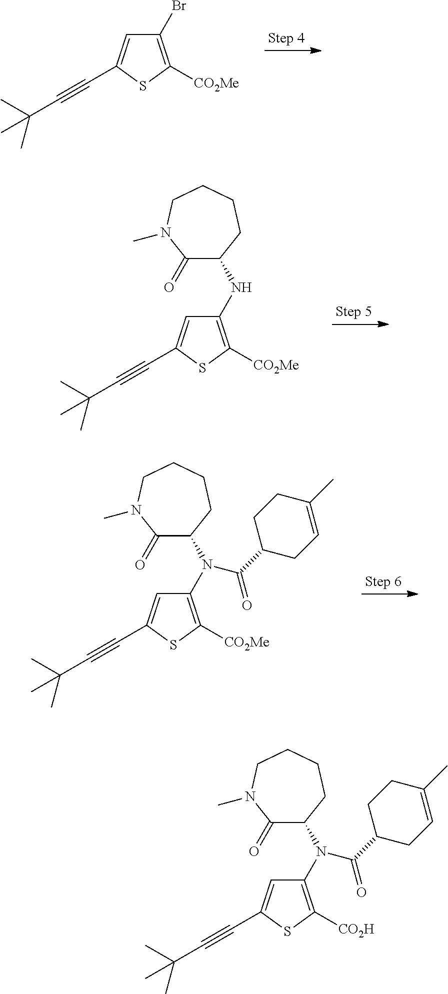

### Compound 179

Steps 4-6 can be performed according to the procedures described in compounds 95, 176.

### Preparation of Compound 180 (Prophetic)

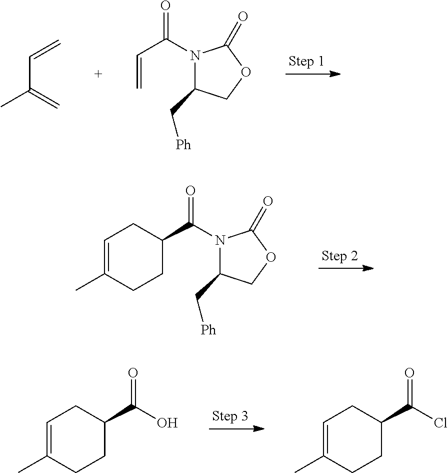

Steps 1, 2:

Steps 1 and 2 can be performed as described in J. Am. Chem. Soc. 1988, 110(4), 1238.

Step 3:

Step 3 can be performed as described for compound 6.

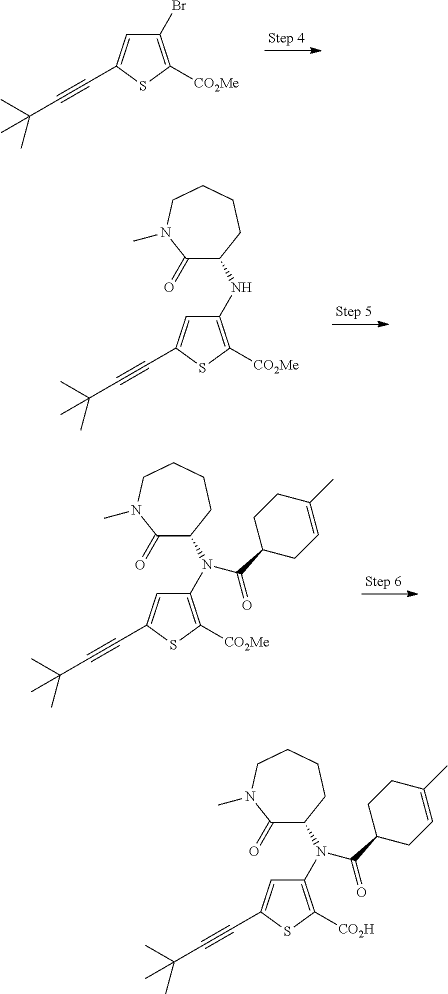

Compound 180

Steps 4-6 can be performed according to the procedures described in compounds 95, 176.

### Preparation of Compound 181 (Prophetic)

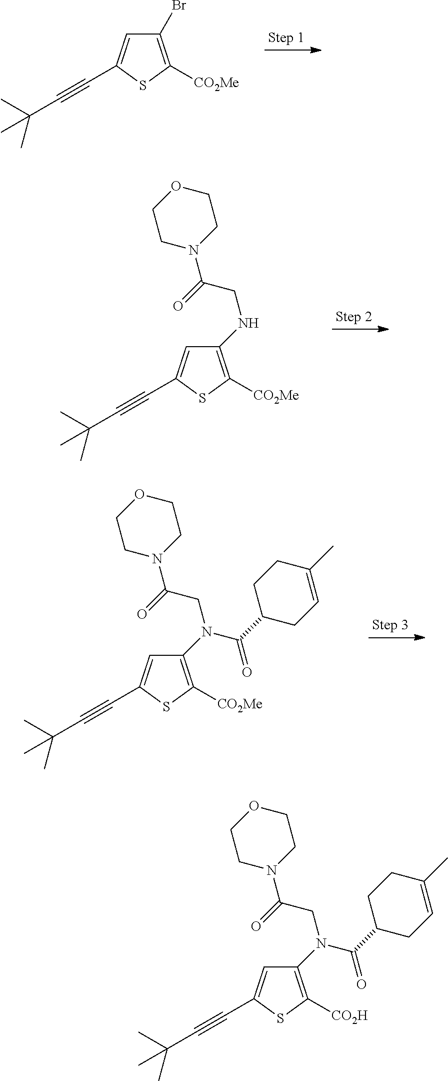

### Compound 181

Steps 1-3 can be performed according to the procedures described in compounds 14, 179.

### Preparation of Compound 182 (Prophetic)

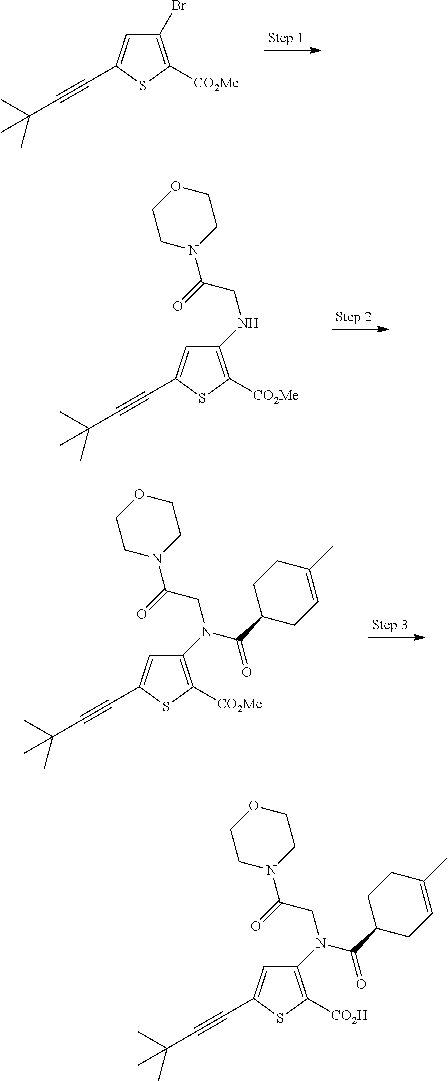

### Compound 182

Steps 1-3 can be performed according to the procedures described in compounds 14, 180.

### Preparation of Compound 183 (Prophetic)

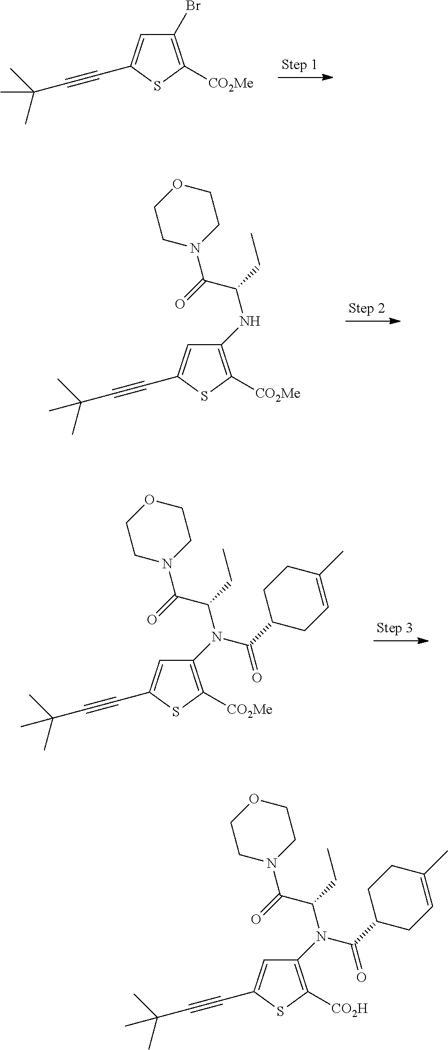

### Compound 183

Steps 1-3 can be performed according to the procedures described in compounds 12, 179.

### Preparation of Compound 184 (Prophetic)

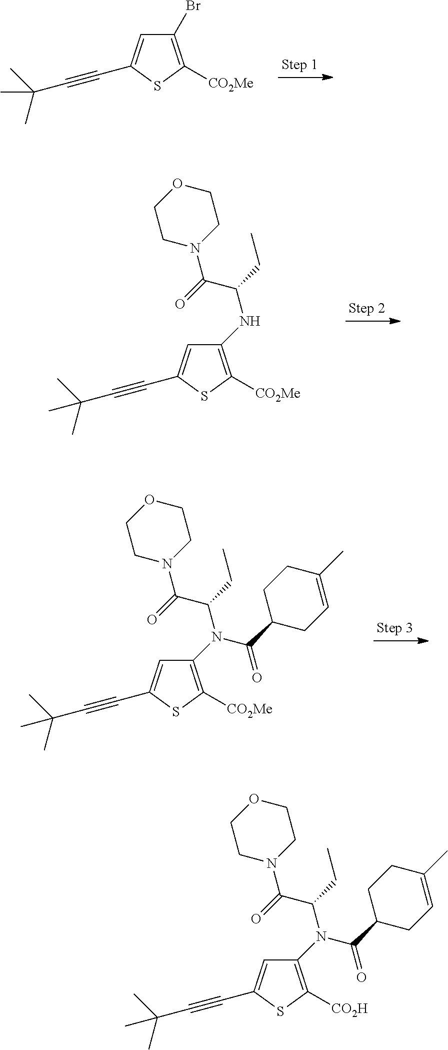

### Compound 186

Steps 1-3 can be performed according to the procedures described in compounds 12, 180.

### Preparation of Compound 185 (Prophetic)

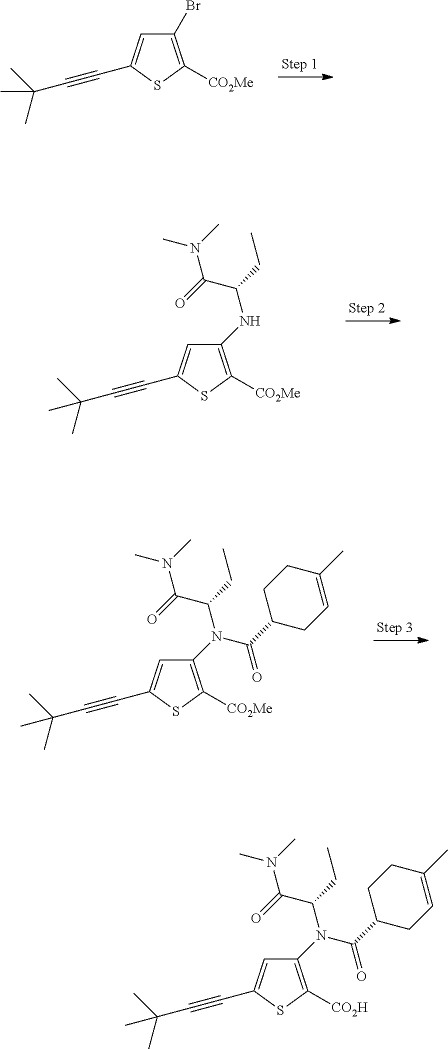

### Compound 185

Steps 1-3 can be performed according to the procedures described in compounds 13, 179.

### Preparation of Compound 186 (Prophetic)

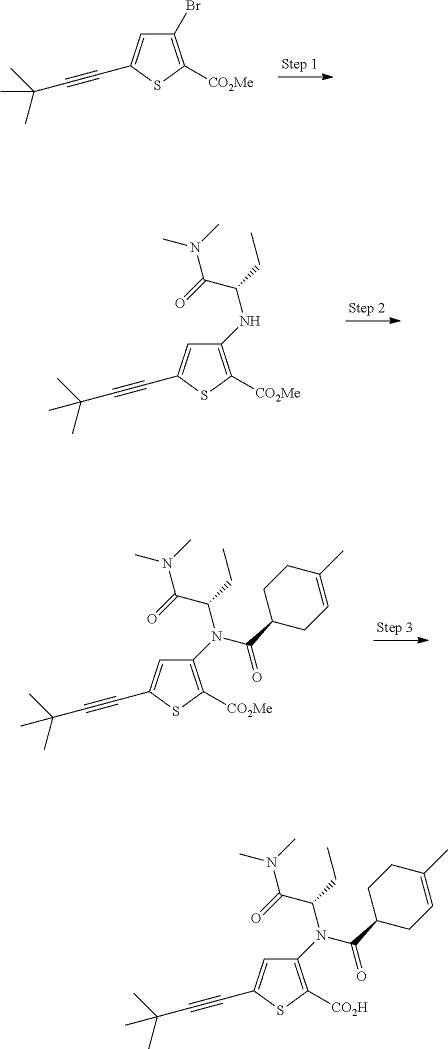

### Compound 186

Steps 1-3 can be performed according to the procedures described in compounds 13, 180.

### Preparation of Compound 187 (Prophetic)

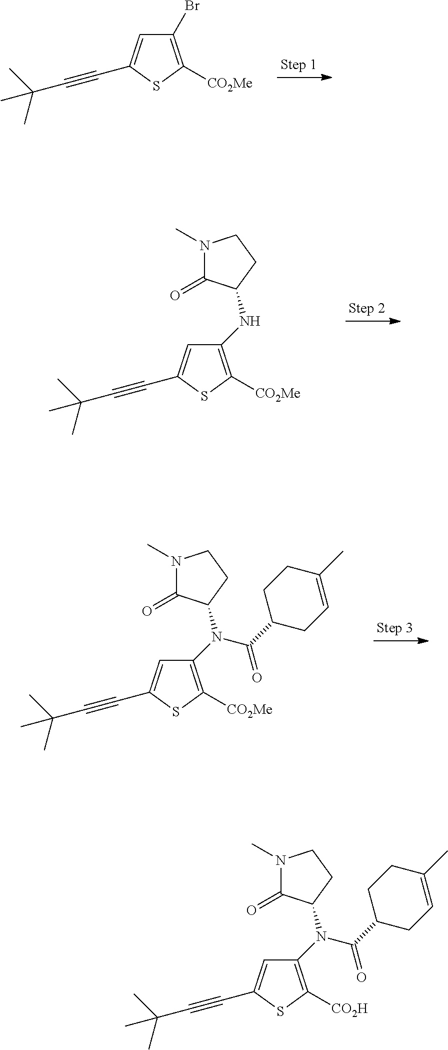

### Compound 187

Steps 1-3 can be performed according to the procedures described in compounds 83, 91, 92, 179.

### Preparation of Compound 188 (Prophetic)

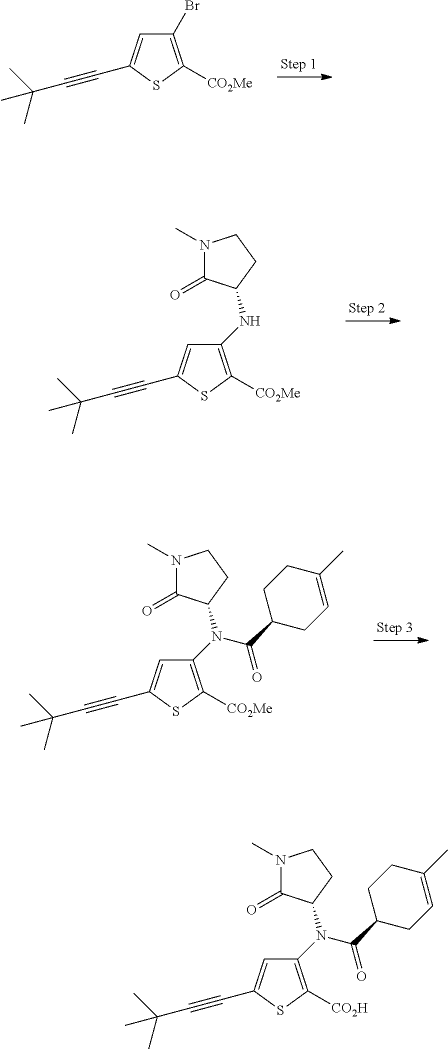

### Compound 188

Steps 1-3 can be performed according to the procedures described in compounds 83, 91, 92, 180.

### Preparation of Compound 189 (Prophetic)

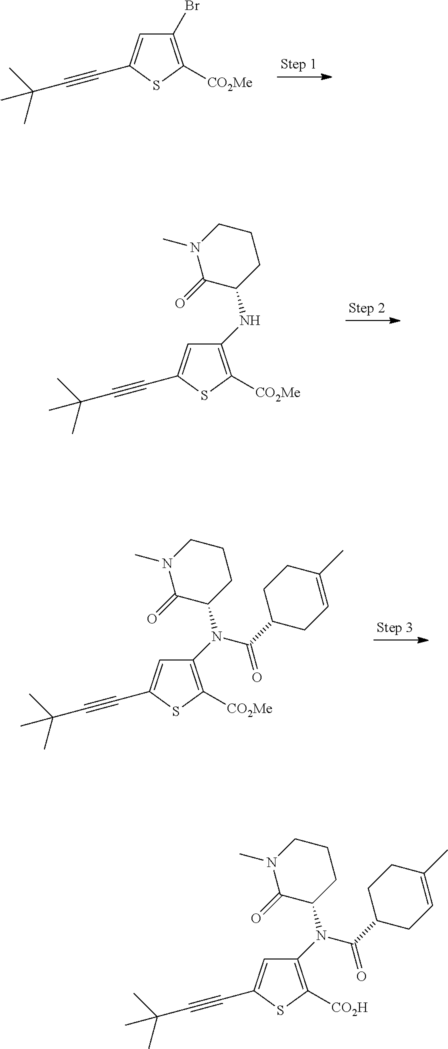

### Compound 189

Steps 1-3 can be performed according to the procedures described in compounds 117, 179.

### Preparation of Compound 190 (Prophetic)

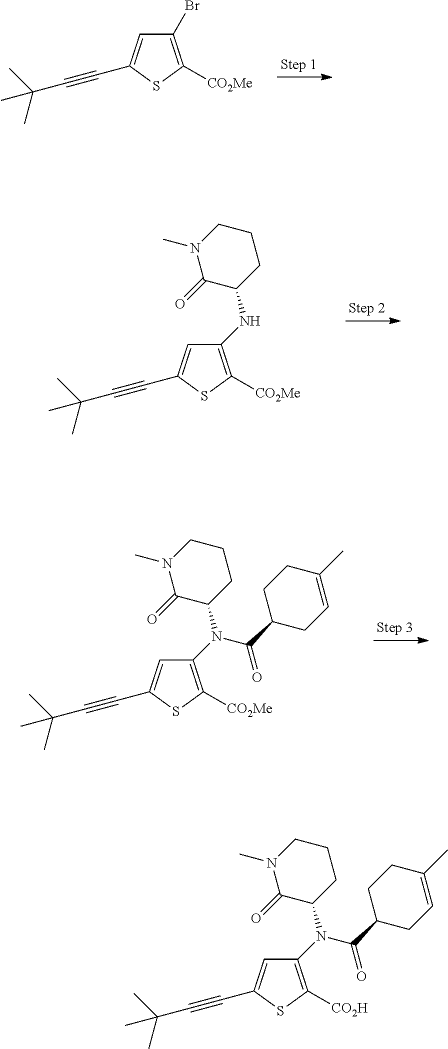

### Compound 190

Steps 1-3 can be performed according to the procedures described in compounds 117, 180.

### Preparation of Compound 191 (Prophetic)

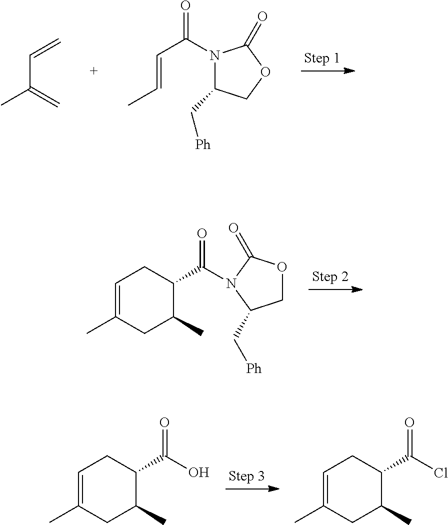

Steps 1, 2:

Steps 1 and 2 can be performed as in J. Am. Chem. Soc. 1988, 110(4), 1238.

Step 3:

Step 3 can be performed as described for compound 6, etc.

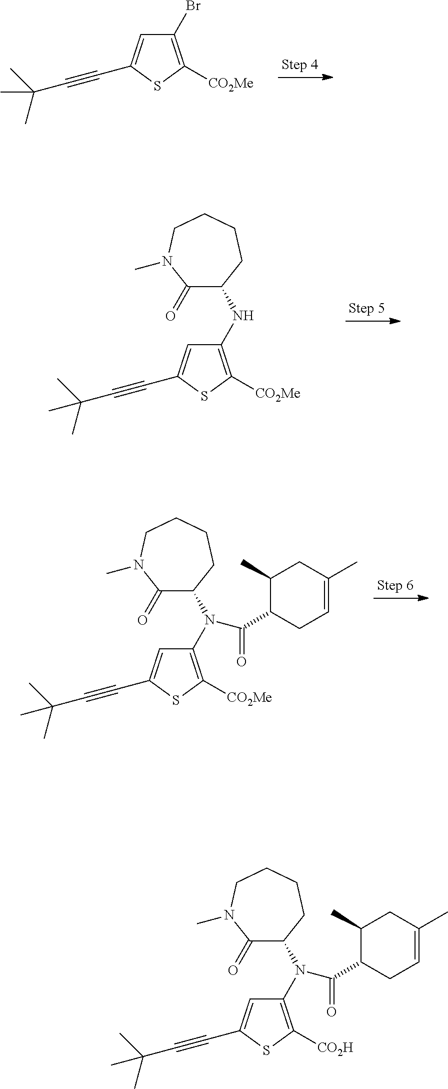

### Compound 191

Steps 4-6 can be performed according to the procedures described in compounds 95, 176.

### Preparation of Compound 192 (Prophetic)

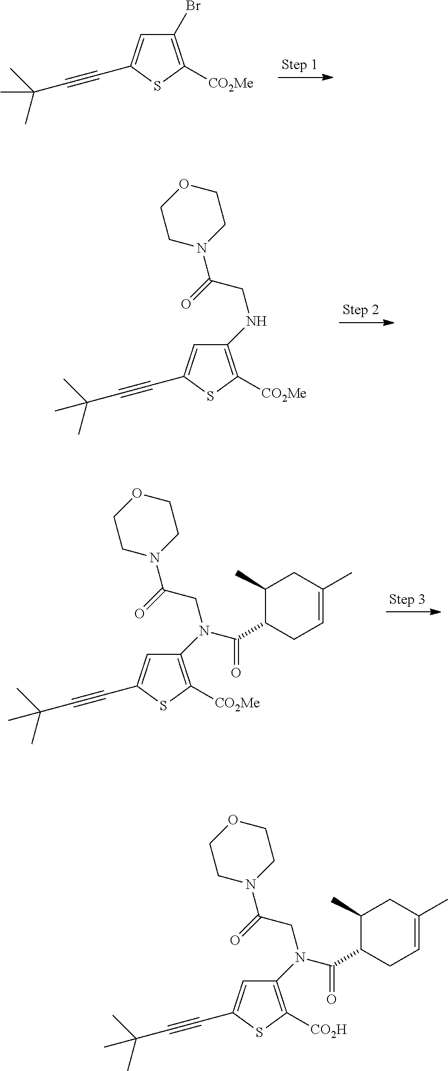

### Compound 192

Steps 1-3 can be performed according to the procedures described in compounds 14, 181, 191.

### Preparation of Compound 193 (Prophetic)

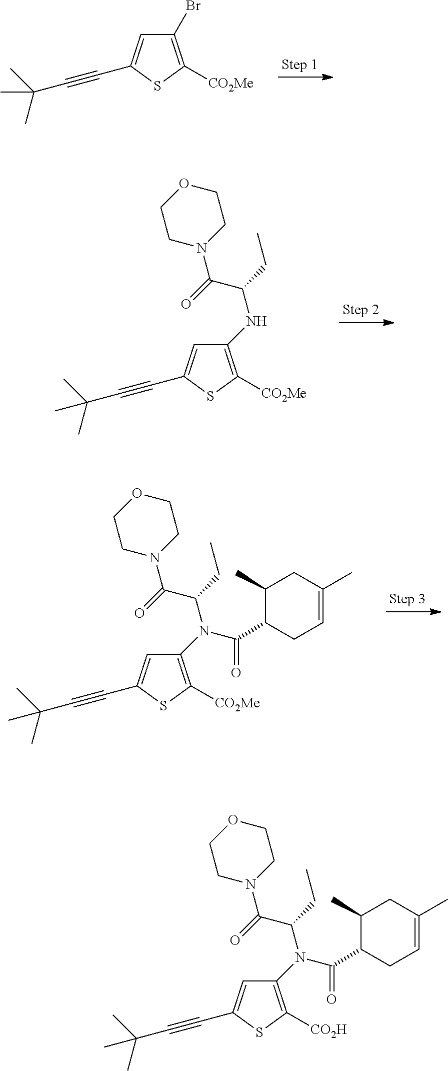

### Compound 193

Steps 1-3 can be performed according to the procedures described in compounds 12, 181, 191.

### Preparation of Compound 194 (Prophetic)

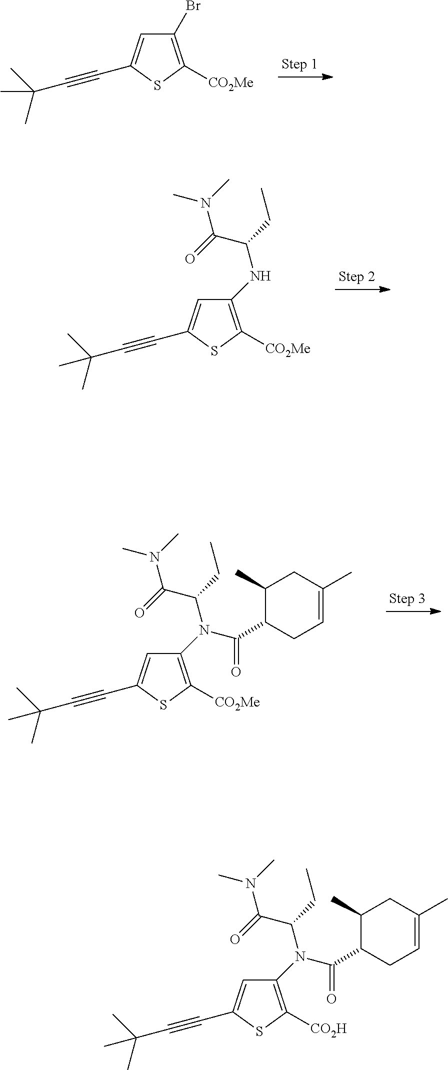

### Compound 194

Steps 1-3 can be performed according to the procedures described in compounds 13, 191.

### Preparation of Compound 195 (Prophetic)

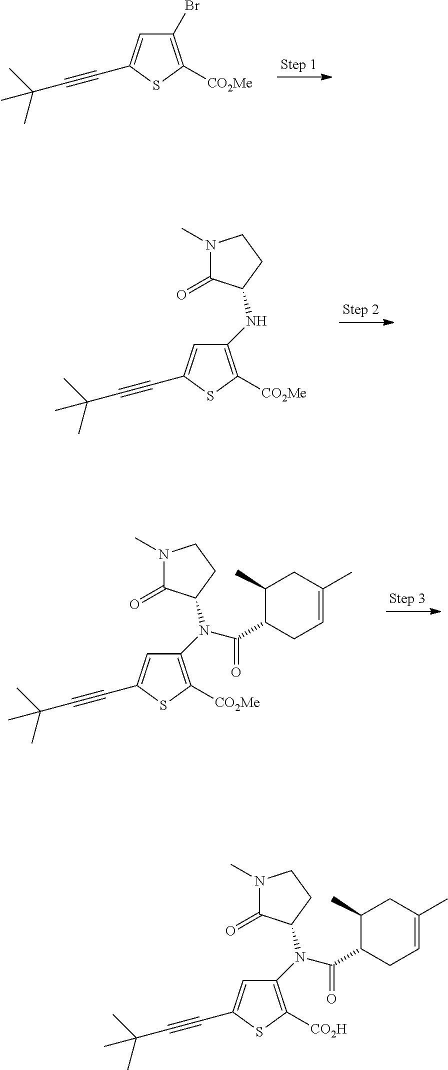

### Compound 195

Steps 1-3 can be performed according to the procedures described in compounds 83, 91, 92, 191.

### Preparation of Compound 196 (Prophetic)

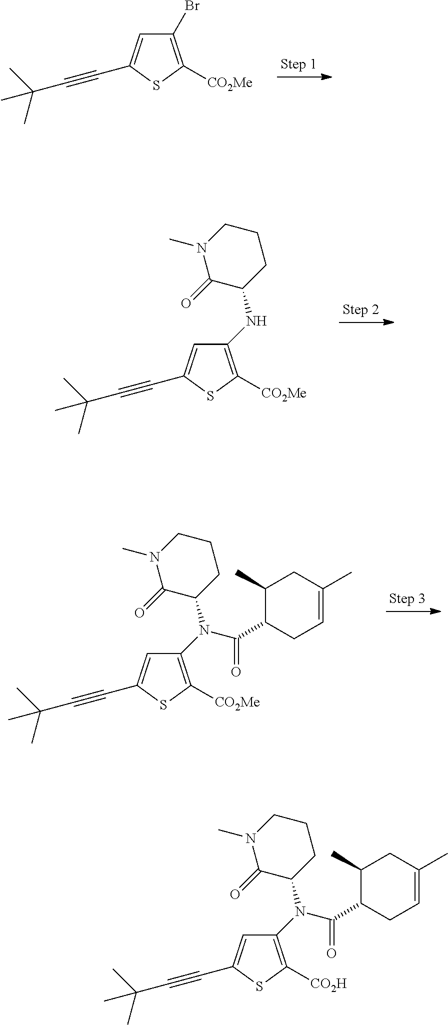

### Compound 196

Steps 1-3 can be performed according to the procedures described in compounds 117, 191.

### Example 2

### PK parameters of Certain Compounds of the Invention

For the determination of PK parameters of certain compounds of the invention, the compounds can be formulated as a solution in 0.5% MC/0.5% Tween 80/99% water and administered orally by gavage to rats at a dose of 3 mg/kg. Rats are weighed the day before the study. Rat plasma is sampled predose and at 15, 30 min, 1, 1.5, 2, 3, 4, 6, 8, 12 and 24 hrs post dose using Instech automatic blood sampling equipment. Blood is collected in tubes containing K2-EDTA and 90 uL plasma are extracted for analysis. Rats are fed ad lib and standard IACUC and SOP protocols are followed. Plasma samples and dose samples are analyzed using LC/MS/MS.

The PK parameters of Compound 160 were measured as described in the preceding paragraph, and the data showed its conversion (over 50%) to Compound 95.

### Example 3

### HCV Replicon Assay

### A. Principle

This procedure below describes the HCV replicon assay using a Huh7 hepatoma cell line harboring a highly cell culture-adapted replicon (genotype 1b) (hereafter named cell line ET). The ET cells contained the highly cell culture-adapted replicon I₃₈₉luc-ubi-neo/NS3-3′/5.1 construct that carried, in addition to the neomycin gene, an integrated copy to the firefly luciferase gene (Krieger, N; Lohmann, V; Bartenschlager, R. Enhancement of hepatitis C virus RNA replication by cell culture-adaptive mutations. J. Virol. 2001, 75, 4614-4624). A replicon cell line W11.8, containing the 1a genotype of HCV was also used. These two cell lines (genotype 1b and 1a) allowed measurement of RNA replication and translation by measuring luciferase activity (against genotype 1b) or by measuring the NS5A level using the ELISA assay (against genotype 1a). It was shown that the luciferase activity tightly followed the replicon RNA level in the ET cells. ET cell lines were maintained in cultures at a sub-confluent level (<85%). The culture media used for cell passaging consisted of DMEM (Gibco BRL Laboratories, Mississauga, ON, Canada) supplemented with 10% fetal bovine serum with 1% penicilin/streptomycin, 1% glutamine, 1% sodium pyruvate, 1% non-essential amino acids, and 180 g/ml of G418 final concentration.

### B. Measurement of Luciferase Activity (Luci-ET-1b)

For the treatment of the cells with the testing drug, the culture medium was removed from the 175 cm² T-flask by aspiration. Cell monolayer was rinsed with 10 mL of PBS 1× at room temperature. PBS was removed by aspiration. Cells were trypsinized using 1 mL of Trypsin/EDTA. Flask were incubated at 37° C. (incubator) for 7 minutes. Complete medium (9 mL) with no G418 and no phenol red was then added. Cell clumps were disrupted by pipetting up and down several times. The cell suspension was then transferred to a 50 mL Falcon polypropylene tube. Cells were then counted several times using the hemacytometer. Cells were diluted at 30 000 cells/mL with complete DMEM with no G418 and no phenol red, then transferred into a sterile reservoir. Using a multichannel pipet, approximately 3000 viable cells (100 μL) were plated per well in a white opaque 96-well microtiter plate. After an incubation period of 2-4 hours at 37° C. in a 5% CO₂ incubator, compounds were added at various concentrations.

Compounds under testing were resuspended in DMSO at a stock concentration of 100 mM. Then, they were diluted at twice the final concentration in the same medium (without G418) described earlier, in sterile 96-deep well plate and according to a particular template. One volume (100 μL) of each compound dilution was then added to each well that contains cells or in control wells with no cells. Final drug concentrations were usually between 200 μM and 0.0001 μM. Ten wells were used as positive control without drug. Cells were further incubated for 4 days at 37° C. in a 5% CO₂ incubator. A control compound was used as an internal standard at the same concentrations described above.

Following the incubation time of four days, the culture media was removed and quickly dried upside down on a stack of sterile absorbing papers. Cells were then lysed by the addition of 95 μL of the luciferase buffer A using a multichannel pipet, sealed using TopSeal™ adhesive sealing film and the reaction mixture was incubated at room temperature and protected from direct light for at least 10 minutes. Plates were read for luciferase counts using a luminometer (Wallac MicroBeta Trilux, Perkin Elmer™, MA, USA).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC₅₀ values are summarized in Tables 1-3:

A: IC₅₀ value (mean) ≦0.1 μM;

B: 0.1 μM ≦IC₅₀ value (mean) ≦1 μM;

C: 1 μM ≦IC₅₀ value (mean) ≦10 μM;

D: IC₅₀ value (mean) >10 μM.

### C. Elisa Assay (ELISA W 11.8-1a)

Replicon cell lines W11.8 containing a sub-genomic replicon of genotype 1a was used for the HCV Replicon Cell-Based detection using the ELISA. The RNA replication in presence of different drug concentrations was indirectly measured in these cell lines by the level of NS5A protein content upon drug treatment for four days. The NS5A is a non-structural protein of HCV and is used as marker of HCV replication in this assay.

For the treatment of the cells with the testing drug, Culture medium was removed from the 175 cm² T-flask by aspiration. Cell monolayer was rinsed with 10-20 mL of PBS 1× at room temperature. PBS was removed by aspiration. Cells were trypsinized using 3 mL of Trypsin (0.25%)/EDTA (0.1%) solution. Flasks were incubated at 37° C. (incubator) for 7 minutes. Complete medium (9 mL) without G418 is then added. Cell clumps were disrupted by pipetting up and down several times.

The cell suspension was then transferred to a 50 mL Falcon polypropylene tube. Cells were then counted several times using the haemocytometer. Cells were diluted at 50,000 cells/mL with complete DMEM without G418, then transferred into a sterile reservoir. Using a multichannel pipet, approximately 5,000 viable cells (100 μL) were plated per well in a white opaque 96-well microtiter plate. After an incubation period of 2-4 hours at 37° C. in a 5% CO₂ incubator, compounds were added at various concentrations.

Drugs were resuspended in DMSO at a stock concentration of 100 mM or 10 mM. In some cases (drugs with a potency below nmolar values), it was necessary to dilute compounds in DMSO at 1 mM or 100 M as a starting solution. Then, drugs were diluted at twice the final concentration in the same medium (without G418) described earlier, in sterile 96-deep well plate and according to a particular template (see Appendix). One volume (100 μL) of each drug dilution was then added to each well that contains cells.

Sixteen wells were used as control (0% inhibition) without drug. Eight wells were used as background control (100% inhibition) containing 2 μM (final concentration) of the reference compound. The reference compound at 2 μM was shown to inhibit the NS5A expression at ≈100% and is nontoxic to the cells. Values from 100% inhibited wells were averaged and used as the background value. Cells are further incubated for 4 days at 37° C. in a 5% CO₂ incubator.

For the measurement of NS5A protein content, following the incubation time of four days, the media was throwed into an appropriate waste container by inverting the plate. Any residual liquid was removed by tapping gently on absorbent paper several times. The plates were then washed once with 150 μL of PBS per well, and then incubated for 5 minutes at room temperature on a shaker (500 rpm). 150 μL per well of cold (−20° C.) fixative solution (50% methanol/50% acetone mix) was added into the plates, and the plates was incubated for 5 minutes at room temperature. The plates were then inverted, and any residual liquid was removed by tapping gently on absorbent paper several times. The plates were then washed twice with 150 μL of PBS per well, and incubated for 5 minutes at room temperature on a shaker (500 rpm) for each wash. 150 μL of blocking solution per well was added into the plates. The plates were then sealed using TopSeal™ adhesive sealing films and incubated for one hour at 37° C. or at 4° C. overnight to block non-specific sites.

The plates were inverted and the blocking solution was dumped into an appropriate waste container. Any residual liquid was removed by tapping gently on absorbent paper several times. The plates were then washed twice with 150 μL of PBS per well and once with 150 μL of PBSTS solution per well, and then incubated for 5 minutes at room temperature on a shaker (500 rpm) for each wash. Then, was add into the plates 50 μL per well of anti-human NS5A antibody (Ab1) diluted 1/1,000 in the blocking solution. The plates were then sealed using TopSeal™ adhesive sealing films and incubate at 4° C. overnight.

Next day, the plates were inverted to dump solution into an appropriate waste container. The plates then were gently tapped on absorbent paper several times to remove residual liquid. The plates were washed five times with 150 μL of PBS per well, and incubated for 5 minutes at room temperature on a shaker (500 rpm) for each wash. Then was add into the plates 50 μL per well of peroxidase-conjugated donkey anti-mouse antibody (Ab2) diluted 1/10,000 in the blocking solution. The plates were then sealed using TopSeal™ adhesive sealing films and incubate at room temperature for 3 hours on a shaker (500 rpm). Towards the end of the 3 hours incubation, the commercially available chemiluminescent substrate solution was prepared. A mixture of equal volumes of the luminol/enhancer and stable peroxide reagents was prepared and protected from light. The plates were then inverted to dump solution into an appropriate waste container. Any residual liquid was removed by tapping gently on absorbent paper several times. The plates were washed four times with 150 μL of PBSTS solution per well and once with 150 μL of PBS, and then incubated for 5 minutes at room temperature on a shaker (500 rpm) for each wash. 100 μL of substrate solution per well was then added into the plates. The plates were then sealed using TopSeal™ adhesive sealing films and incubate for 1 minute at room temperature on a shaker (500 rpm), and then incubated between 30 and 60 minutes at room temperature (protect from light) prior to reading the luminescence (relative light units) on the Analyst HT plate reader (LJL Default Luminescence Method).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC50 values are summarized in Tables 1-3:

A: IC₅₀ value (mean) ≦0.1 μM;

B: 0.1 μM <IC₅₀ value (mean) ≦1 μM;

C: 1 μM <IC₅₀ value (mean) ≦10 μM;

D: IC₅₀ value (mean) >10 μM.

### Example 4

### [³H]Thymidine Incorporation Assay

A total of 2,000 cells/well were seeded in 96-well cluster dishes in a volume of 100 [mu]l of DMEM (Wisent., St Bruno, QC) supplemented with 10% FBS (Wisent., St Bruno, QC) and 2 mM glutamine (Life Technologies, Inc.). Penicillin and streptomycin (Life Technologies, Inc.) are added to 500 U/mL and 50 g/mL final concentrations, respectively. After an incubation of at least 3 h at 37° C. in an atmosphere of 5% CO₂, compounds, prepared at twice the final concentration, are added to the cells. Eleven serial two to four-fold dilutions of drugs are tested in duplicate plates. After 72-h incubation, a volume of 20 μL of a 10 μCi/mL solution of [3H]methyl thymidine (Amersham Life Science, Inc., Arlington Heights, Ill.; 2 Ci/mmol) in culture medium is added and the plates are incubated for a further a 24 h at 37° C. Cells are then washed with phosphate-buffered saline (PBS), trypsinized for 2 min, and collected onto a fiberglass filter using a Tomtec cell harvester (Tomtec, Orange, Conn.). Filters are dried at 37° C. for 1 h and placed into a bag with 4.5 mL of liquid scintillation cocktail (Wallac Oy, Turku, Finland). The accumulation of [3H]methyl thymidine, representing viable replicating cells, is measured using a liquid scintillation counter (1450-Microbeta; Wallac Oy). Ref. SOP: 265-162-03. For this experiment, the cell lines used are; Huh-7 ET (cells derived from the Huh-7 cell line (hepatocellular carcinoma, human) and containing a HCV sub-genomic replicon), Molt-4 (peripheral blood, acute lymphoblastic leukemia, human), DU-145 (prostate carcinoma, metastasis to brain, human), Hep-G2 (hepatocellular carcinoma, human), and SH-SY5Y (neuroblastoma, human) cells.

The 50% cytotoxic concentrations (CC₅₀) for cell toxicity were determined from dose response curves using six to eight concentrations per compound in triplicate. Curves were fitted to data points using non-linear regression analysis, and IC₅₀ values were interpolated from the resulting curve using GraphPad Prism software, version 2.0 (GraphPad Software Inc., San Diego, Calif., USA).

CC₅₀ values of compounds of the invention are summaries in Tables 1-3:

A: CC₅₀ value (mean) ≧100 μM;

B: 10 μM≦CC₅₀ value (mean) <100 μM;

C: CC₅₀ value (mean) ≦10 μM.

TABLE 1
IC₅₀, CC₅₀ LCMS and NMR data of the compounds described in FIG. 1

| | HCV-Replicon- | HCV-Replicon- | | | | |
|---|---|---|---|---|---|---|
| Compound | (Luci-ET) | ELISA-1a | [³H]Thymidine | LCMS | LCMS | |
| Nos. | 1b_IC50 | IC50 | CC50 | [M + H]⁺ | RT | NMR |
| 1 | A | | B | 419.60 | | |
| 2 | A | | B | 447.2 | | ¹H NMR (400 |
| | | | | | | MHz, DMSO): δ |
| | | | | | | 7.05 (s, 1H), 5.22 |
| | | | | | | (q, 1H), 3.01 (s, |
| | | | | | | 3H), 2.81 (s, 3H), |
| | | | | | | 1.99 (t, 1H), 1.76- |
| | | | | | | 1.00 (m, 16H), |
| | | | | | | 0.90 (d, 3H), |
| | | | | | | 0.73 (d, 3H), |
| | | | | | | 0.69-0.35 (m, |
| | | | | | | 2H) |
| 3 | A | A | A | 447.43 | 5.24 | |
| 4 | A | B | B | 480.93 | | |
| 5 | A | A | A | 433.54 | 4.48 | |
| 6 | A | A | A | 459.46 | 1.62 | |
| 7 | A | | B | 447 | | |
| 8 | A | | A | 475.5 | 5.38 | 1H NMR (300 |
| | | | | | | MHz, CDCl3) d |
| | | | | | | 6.62 (s, 1H), 4.81 |
| | | | | | | (d, J = 6.6 Hz, |
| | | | | | | 1H), 3.65-2.95 |
| | | | | | | (m, 5H), 2.07- |
| | | | | | | 0.19 (m, 31H). |
| 9 | A | | A | 473.5 | 5.38 | H NMR (300 |
| | | | | | | MHz, CDCl3) d |
| | | | | | | 6.63 (s, 1H), 4.68 |
| | | | | | | (d, J = 7.2 Hz, |
| | | | | | | 1H), 3.93-3.22 |
| | | | | | | (m, 5H), 2.17- |
| | | | | | | 0.44 (m, 29H). |
| 10 | A | | A | 487.5 | 5.5 | |
| 11 | A | | A | 489.53 | 5.69 | |
| 12 | A | A | B | 503.54 | 5.6 | 1H NMR (300 |
| | | | | | | MHz, DMSO) d |
| | | | | | | 13.53 (s, 1H), |
| | | | | | | 7.27 (s, 0.6H), |
| | | | | | | 7.13 (s, 0.4H), |
| | | | | | | 5.56 (t, J = 7.4 |
| | | | | | | Hz, 0.6H), 5.20 |
| | | | | | | (t, J = 7.4 Hz, |
| | | | | | | 0.4H), 3.53 (qdd, |
| | | | | | | J = 27.8, 20.6, |
| | | | | | | 13.1 Hz, 8H), |
| | | | | | | 3.19-2.99 (m, |
| | | | | | | 0.4H), 1.98 (br, |
| | | | | | | 0.6H), 1.79- |
| | | | | | | 1.05 (m, 18H), |
| | | | | | | 0.92-0.38 (m, |
| | | | | | | 8H). |
| 13 | A | A | A | 461.51 | 5.28 | |
| 14 | A | A | A | 475.45 | 5.21 | 1H NMR (300 |
| | | | | | | MHz, CDCl3) d |
| | | | | | | 7.19 (s, 1H), 6.90 |
| | | | | | | (s, 1H), 4.68 (d, |
| | | | | | | J = 14.7 Hz, 1H), |
| | | | | | | 4.24-3.99 (m, |
| | | | | | | 2H), 3.59 (d, J = |
| | | | | | | 17.3 Hz, 9H), |
| | | | | | | 3.40 (d, J = 3.3 |
| | | | | | | Hz, 3H), 2.18 (d, |
| | | | | | | J = 11.3 Hz, 1H), |
| | | | | | | 2.00 (d, J = 14.4 |
| | | | | | | Hz, 0H), 1.72 (d, |
| | | | | | | J = 12.1 Hz, 1H), |
| | | | | | | 1.63-1.40 (m, |
| | | | | | | 6H), 1.37-1.11 |
| | | | | | | (m, 18H). |
| 15 | A | A | A | 489.4 | 5.36 | 1H NMR (300 |
| | | | | | | MHz, CDCl3) δ |
| | | | | | | 7.49 (s, 1H), 6.72 |
| | | | | | | (s, 2H), 5.39 (q, |
| | | | | | | J = 7.3 Hz, 1H), |
| | | | | | | 5.06 (q, J = 7.5 |
| | | | | | | Hz, 2H), 4.02- |
| | | | | | | 3.55 (m, 21H), |
| | | | | | | 2.06 (dd, J = |
| | | | | | | 13.5, 9.8 Hz, |
| | | | | | | 3H), 1.81 (d, J = |
| | | | | | | 13.1 Hz, 1H), |
| | | | | | | 1.74-1.41 (m, |
| | | | | | | 12H), 1.34 (d, |
| | | | | | | J = 3.2 Hz, 21H), |
| | | | | | | 1.19 (d, J = 7.4 |
| | | | | | | Hz, 4H), 1.05 (d, |
| | | | | | | J = 7.4 Hz, 3H), |
| | | | | | | 0.88-0.59 (m, |
| | | | | | | 12H). |
| 16 | A | A | A | 463.36 | 4.9 | 1H NMR (300 |
| | | | | | | MHz, DMSO) δ |
| | | | | | | 7.25 (s, 0.5H), |
| | | | | | | 7.00 (s, 0.5H), |
| | | | | | | 5.80 (t, 0.5H), |
| | | | | | | 5.35 (t, 0.5H), |
| | | | | | | 3.48 (d, 1H), |
| | | | | | | 3.42-2.75 (m, |
| | | | | | | 11H), 2.01-1.34 |
| | | | | | | (m, 6H), 1.20 (d, |
| | | | | | | 6H), 1.25-1.15 |
| | | | | | | (m, 2H), 0.75 (d, |
| | | | | | | 3H), 0.76-0.50 |
| | | | | | | (m, 2H). |
| 17 | A | A | A | 461.44 | 1.87 | |
| 18 | A | | B | 487 | 3.97 | 1H NMR (300 |
| | | | | | | MHz, CDCl3) δ |
| | | | | | | 7.08-6.74 (m, |
| | | | | | | 1H), 6.63 (d, J = |
| | | | | | | 53.1 Hz, 1H), |
| | | | | | | 4.89-4.56 (m, |
| | | | | | | 1H), 4.19 (m, |
| | | | | | | 1H), 3.83 (m, |
| | | | | | | 1H), 2.21-1.87 |
| | | | | | | (m, 2H), 1.86- |
| | | | | | | 1.54 (m, 8H), |
| | | | | | | 1.52-1.04 (m, |
| | | | | | | 14H), 1.05-0.56 |
| | | | | | | (m, 7H). |
| 19 | A | | A | 503 | 3.2 | 1H NMR (300 |
| | | | | | | MHz, CDCl3) δ |
| | | | | | | 6.90 (s, 1H), 5.39 |
| | | | | | | (s, 1H), 4.44 (m, |
| | | | | | | 1H), 4.12-3.77 |
| | | | | | | (m, 5H), 3.59- |
| | | | | | | 3.31 (m, 3H), |
| | | | | | | 2.83 (s, 3H), 2.14- |
| | | | | | | 1.74 (m, 4H), |
| | | | | | | 1.71-1.14 (m, |
| | | | | | | 15H), 1.09-0.56 |
| | | | | | | (m, 5H). |
| 20 | A | | A | 503 | 2.97 | 1H NMR (300 |
| | | | | | | MHz, CDCl3) δ |
| | | | | | | 6.61 (s, 1H), 5.08- |
| | | | | | | 4.90 (m, 1H), |
| | | | | | | 4.04 (s, 1H), 3.95- |
| | | | | | | 3.04 (m, 6H), |
| | | | | | | 2.22-1.31 (m, |
| | | | | | | 12H), 1.31-1.14 |
| | | | | | | (m, 10H), 1.10 (t, |
| | | | | | | J = 7.9 Hz, 2H), |
| | | | | | | 1.02-0.44 (m, |
| | | | | | | 6H). |
| 21 | A | A | A | 477.33 | 4.21 | 1H NMR (300 |
| | | | | | | MHz, CDCl3) δ |
| | | | | | | 7.65 (s, 0.4H), |
| | | | | | | 6.80 (s, 0.6H), |
| | | | | | | 5.60 (dd, 0.4H), |
| | | | | | | 5.20 (t, 0.6H), |
| | | | | | | 3.52-3.00 (m, |
| | | | | | | 11H), 2.11-1.90 |
| | | | | | | (m, 1H), 1.85- |
| | | | | | | 1.35 (m, 7H), |
| | | | | | | 1.30 (s, 9H), 0.80 |
| | | | | | | (d, 3H), 0.78- |
| | | | | | | 0.60 (m, 2H). |
| 22 | A | | A | 476.78 | 4.25 | 1H NMR (300 |
| | | | | | | MHz, CDCl3) δ |
| | | | | | | 7.62 (s, 0.4H), |
| | | | | | | 6.80 (s, 0.6H), |
| | | | | | | 5.61 (dd, 0.4H), |
| | | | | | | 5.23 (t, 0.6H), |
| | | | | | | 3.52-3.00 (m, |
| | | | | | | 11H), 2.11-1.90 |
| | | | | | | (m, 1H), 1.85- |
| | | | | | | 1.35 (m, 7H), |
| | | | | | | 1.30 (s, 9H), 0.80 |
| | | | | | | (d, 3H), 0.78- |
| | | | | | | 0.60 (m, 2H). |
| 23 | A | A | A | 519.35 | 3.78 | 1H NMR (300 |
| | | | | | | MHz, DMSO) δ |
| | | | | | | 13.43 (s, 1H), |
| | | | | | | 7.28 (s, 0.5H), |
| | | | | | | 7.07 (s, 0.5H), |
| | | | | | | 5.83 (t, J = 7.2 |
| | | | | | | Hz, 0.5H), 5.41 |
| | | | | | | (t, J = 6.7 Hz, |
| | | | | | | 0.5H), 3.79- |
| | | | | | | 2.89 (m, 14H), |
| | | | | | | 2.11-1.00 (m, |
| | | | | | | 17H), 0.87-0.39 |
| | | | | | | (m, 4H). |
| 24 | A | | A | 505.33 | 3.56 | 1H NMR (300 |
| | | | | | | MHz, DMSO) δ |
| | | | | | | 13.48 (brs, 1H), |
| | | | | | | 7.28 (s, 0.5H), |
| | | | | | | 7.01 (s, 0.5H), |
| | | | | | | 5.83 (t, 0.5H), |
| | | | | | | 5.46 (t, 0.5H), |
| | | | | | | 3.70-2.99 (m, |
| | | | | | | 13H), 2.11-0.76 |
| | | | | | | (m, 11H), 1.30 |
| | | | | | | (s, 9H). |
| 25 | A | | A | 489.1 | 3.53 | 1H NMR (300 |
| | | | | | | MHz, CDCl3) δ |
| | | | | | | 6.91 (d, J = 1.7 |
| | | | | | | Hz, 1H), 5.11 (d, |
| | | | | | | J = 16.4 Hz, 1H), |
| | | | | | | 4.00 (s, 2H), 3.91- |
| | | | | | | 3.80 (m, 1H), |
| | | | | | | 3.77-3.61 (m, |
| | | | | | | 1H), 3.59-3.45 |
| | | | | | | (m, 1H), 3.43- |
| | | | | | | 3.19 (m, 3H), |
| | | | | | | 2.32 (t, J = 11.6 |
| | | | | | | Hz, 1H), 2.08- |
| | | | | | | 1.81 (m, 3H), |
| | | | | | | 1.60 (dd, J = |
| | | | | | | 17.4, 13.8 Hz, |
| | | | | | | 6H), 1.34 (s, |
| | | | | | | 10H), 0.82 (d, |
| | | | | | | J = 6.5 Hz, 4H), |
| | | | | | | 0.75-0.63 (m, |
| | | | | | | 1H). |
| 26 | A | | B | 489.1 | 4.61 | 1H NMR (300 |
| | | | | | | MHz, CDCl3) δ |
| | | | | | | 6.92 (dd, J = 8.9, |
| | | | | | | 2.1 Hz, 2H), 5.13 |
| | | | | | | (dd, J = 16.4, 7.0 |
| | | | | | | Hz, 1H), 4.95 |
| | | | | | | (dd, J = 16.3, 3.7 |
| | | | | | | Hz, 1H), 4.37 |
| | | | | | | (dd, J = 20.2, |
| | | | | | | 13.5 Hz, 2H), |
| | | | | | | 4.07 (dd, J = |
| | | | | | | 16.5, 5.5 Hz, |
| | | | | | | 1H), 3.94 (d, J = |
| | | | | | | 16.1 Hz, 3H), |
| | | | | | | 3.58 (d, J = 11.9 |
| | | | | | | Hz, 6H), 3.43- |
| | | | | | | 3.22 (m, 3H), |
| | | | | | | 3.08-2.76 (m, |
| | | | | | | 3H), 2.61-2.42 |
| | | | | | | (m, 1H), 2.38- |
| | | | | | | 2.23 (m, 2H), |
| | | | | | | 1.84 (s, 2H), 1.60 |
| | | | | | | (s, 6H), 1.41 (d, |
| | | | | | | J = 10.9 Hz, 1H), |
| | | | | | | 1.34 (s, 13H), |
| | | | | | | 1.27-1.16 (m, |
| | | | | | | 5H), 0.82 (d, J = |
| | | | | | | 6.5 Hz, 5H), 0.71 |
| | | | | | | (dd, J = 18.5, 6.4 |
| | | | | | | Hz, 2H). |
| 27 | A | | A | 503.1 | 4.76 | 1H NMR (300 |
| | | | | | | MHz, CDCl3) δ |
| | | | | | | 6.91 (d, J = 4.4 |
| | | | | | | Hz, 1H), 5.04 |
| | | | | | | (dd, J = 20.6, |
| | | | | | | 16.2 Hz, 1H), |
| | | | | | | 3.95 (s, 1H), 3.51 |
| | | | | | | (dd, J = 152.6, |
| | | | | | | 9.6 Hz, 8H), 2.32 |
| | | | | | | (td, J = 11.9, 3.2 |
| | | | | | | Hz, 1H), 1.87 (d, |
| | | | | | | J = 13.3 Hz, 1H), |
| | | | | | | 1.60 (s, 4H), 1.41- |
| | | | | | | 1.18 (m, 16H), |
| | | | | | | 0.82 (d, J = 6.6 |
| | | | | | | Hz, 5H). |
| 28 | A | | A | 461.6 | 3.21 | 1H NMR (300 |
| | | | | | | MHz, CDCl3) δ |
| | | | | | | 6.96 (d, J = 9.7 |
| | | | | | | Hz, 1H), 4.80- |
| | | | | | | 4.65 (m, 1H), |
| | | | | | | 4.64-3.82 (m, |
| | | | | | | 8H), 2.26 (dd, |
| | | | | | | J = 11.6, 8.1 Hz, |
| | | | | | | 1H), 1.77 (s, 1H), |
| | | | | | | 1.63 (d, J = 11.6 |
| | | | | | | Hz, 4H), 1.34 (s, |
| | | | | | | 11H), 0.82 (d, |
| | | | | | | J = 6.5 Hz, 3H), |
| | | | | | | 0.72 (dd, J = |
| | | | | | | 17.1, 8.1 Hz, |
| | | | | | | 2H). |
| 29 | A | | A | 489.23 | 3.14 | 1H NMR (300 |
| | | | | | | MHz, CDCl3) δ |
| | | | | | | 6.83 (s, 1H), 3.97 |
| | | | | | | (m, 9H), 2.02 (m, |
| | | | | | | 4H), 1.84-1.41 |
| | | | | | | (m, 6H), 1.39- |
| | | | | | | 1.09 (m, 12H), |
| | | | | | | 0.78 (t, J = 13.0 |
| | | | | | | Hz, 5H). |
| 30 | A | | A | 488.99 | 3.35 | 1H NMR (300 |
| | | | | | | MHz, CDCl3) δ |
| | | | | | | 7.19 (s, 0.6H), |
| | | | | | | 6.83 (s, 0.4H), |
| | | | | | | 4.91-4.31 |
| | | | | | | (m, 3H), 4.26- |
| | | | | | | 3.59 (m, 4H), |
| | | | | | | 2.88 (m, 5H), |
| | | | | | | 2.43-1.98 (m, |
| | | | | | | 3H), 1.94-1.45 |
| | | | | | | (m, 5H), 1.45- |
| | | | | | | 0.98 (m, 11H), |
| | | | | | | 0.93-0.46 (m, |
| | | | | | | 4H). |
| 31 | A | | A | 517.7 | 2.99 | 1H NMR (300 |
| | | | | | | MHz, CDCl3) δ |
| | | | | | | 6.98-6.57 (m, |
| | | | | | | 1H), 4.59 (d, J = |
| | | | | | | 7.4 Hz, 1H), 4.29 |
| | | | | | | (d, J = 7.2 Hz, |
| | | | | | | 1H), 4.05 (q, J = |
| | | | | | | 7.2 Hz, 1H), 3.61 |
| | | | | | | (m, 3H), 2.82 (m, |
| | | | | | | 6H), 2.08-1.80 |
| | | | | | | (m, 4H), 1.77- |
| | | | | | | 0.98 (m, 15H), |
| | | | | | | 0.86-0.49 (m, |
| | | | | | | 4H). |
| 32 | A | | A | 489.62 | 3.01 | |
| 33 | A | | A | 505.64 | 2.64 | |
| 34 | A | | A | 463.62 | 3.16 | |
| 35 | A | A | B | 503.35 | 3.81 | 1H NMR (300 |
| | | | | | | MHz, CDCl3) δ |
| | | | | | | 7.65 (s, 0.4H), |
| | | | | | | 6.80 (s, 0.6H), |
| | | | | | | 5.60 (dd, 0.4H), |
| | | | | | | 5.20 (t, 0.6H), |
| | | | | | | 3.52-3.00 (m, |
| | | | | | | 11H), 2.11-1.90 |
| | | | | | | (m, 1H), 1.85- |
| | | | | | | 1.35 (m, 7H), |
| | | | | | | 1.30 (s, 9H), 0.80 |
| | | | | | | (d, 3H), 0.78- |
| | | | | | | 0.60 (m, 2H). |
| 36 | B | | A | 489.34 | 3.61 | 1H NMR (300 |
| | | | | | | MHz, CDCl3) δ |
| | | | | | | 7.62 (s, 0.4H), |
| | | | | | | 6.80 (s, 0.6H), |
| | | | | | | 5.61 (dd, 0.4H), |
| | | | | | | 5.23 (t, 0.6H), |
| | | | | | | 3.52-3.00 (m, |
| | | | | | | 11H), 2.11-1.90 |
| | | | | | | (m, 1H), 1.85- |
| | | | | | | 1.35 (m, 7H), |
| | | | | | | 1.30 (s, 9H), 0.80 |
| | | | | | | (d, 3H), 0.78- |
| | | | | | | 0.60 (m, 2H). |
| 37 | A | | A | 477.1 | 1.11 | 1H NMR (300 |
| | | | | | | MHz, CDCl3) δ |
| | | | | | | 6.63 (s, 1H), 5.33- |
| | | | | | | 4.99 (m, 1H), |
| | | | | | | 4.86 (m, 1H), |
| | | | | | | 4.21-3.43 (m, |
| | | | | | | 4H), 3.32-2.75 |
| | | | | | | (m, 4H), 2.18- |
| | | | | | | 0.29 (m, 25H). |
| 38 | A | | A | 491.1 | 1.55 | |
| 39 | A | | A | 503.1 | 1 | 1H NMR (300 |
| | | | | | | MHz, CDCl3) δ |
| | | | | | | 6.64 (s, 1H), 5.06- |
| | | | | | | 4.56 (m, 1H), |
| | | | | | | 3.99 (m, 1H), |
| | | | | | | 3.51 (m, 5H), |
| | | | | | | 2.87-2.27 (m, |
| | | | | | | 4H), 1.95 (m, |
| | | | | | | 3H), 1.78-0.78 |
| | | | | | | (m, 19H), 0.70 |
| | | | | | | (m, 4H). |
| 40 | A | | A | 517.1 | 1.77 | H NMR (300 |
| | | | | | | MHz, CDCl3) δ |
| | | | | | | 6.80-6.62 (m, |
| | | | | | | 1H), 5.13-4.92 |
| | | | | | | (m, 1H), 3.97- |
| | | | | | | 3.25 (m, 7H), |
| | | | | | | 2.91 (s, 3H), 2.16- |
| | | | | | | 1.40 (m, 11H), |
| | | | | | | 1.38-1.03 (m, |
| | | | | | | 16H), 0.99-0.49 |
| | | | | | | (m, 8H). |
| 41 | C | | A | 503.1 | 4.12 | 1H NMR (300 |
| | | | | | | MHz, CDCl3) δ |
| | | | | | | 7.49 (d, J = 8.0 |
| | | | | | | Hz, 1H), 6.91 (s, |
| | | | | | | 1H), 4.65 (d, J = |
| | | | | | | 16.5 Hz, 1H), |
| | | | | | | 3.82 (d, J = 16.5 |
| | | | | | | Hz, 1H), 3.65 (d, |
| | | | | | | J = 11.0 Hz, 2H), |
| | | | | | | 2.26 (t, J = 11.5 |
| | | | | | | Hz, 1H), 1.69 |
| | | | | | | (dd, J = 68.0, |
| | | | | | | 56.8 Hz, 9H), |
| | | | | | | 1.47-1.08 (m, |
| | | | | | | 15H), 0.81 (d, |
| | | | | | | J = 6.4 Hz, 3H), |
| | | | | | | 0.76-0.61 (m, |
| | | | | | | 2H). |
| 42 | A | | A | 474.94 | 3.34 | 1H NMR (300 |
| | | | | | | MHz, CDCl3) δ |
| | | | | | | 6.92 (dd, J = 8.9, |
| | | | | | | 3.5 Hz, 1H), 5.07- |
| | | | | | | 4.69 (m, 2H), |
| | | | | | | 4.55 (dd, J = |
| | | | | | | 25.5, 11.3 Hz, |
| | | | | | | 1H), 3.92 (t, J = |
| | | | | | | 19.9 Hz, 2H), |
| | | | | | | 3.58 (s, 4H), 2.30 |
| | | | | | | (dd, J = 11.9, 8.2 |
| | | | | | | Hz, 2H), 2.17- |
| | | | | | | 1.91 (m, 2H), |
| | | | | | | 1.84 (d, J = 12.8 |
| | | | | | | Hz, 1H), 1.73- |
| | | | | | | 1.45 (m, 4H), |
| | | | | | | 1.29 (d, J = 19.4 |
| | | | | | | Hz, 10H), 0.80 |
| | | | | | | (d, J = 6.5 Hz, |
| | | | | | | 5H). |
| 43 | A | | A | 502.99 | 3.34 | |
| 44 | A | | A | 502.98 | 3.5 | |
| 45 | | | | 474.96 | 3.8 | 1H NMR (300 |
| | | | | | | MHz, CDCl3) δ |
| | | | | | | 6.91 (d, J = 21.0 |
| | | | | | | Hz, 1H), 4.82- |
| | | | | | | 4.39 (m, 2H), |
| | | | | | | 4.40-3.65 (m, |
| | | | | | | 6H), 3.32 (s, 2H), |
| | | | | | | 2.65 (s, 0H), 2.25 |
| | | | | | | (d, J = 11.5 Hz, |
| | | | | | | 1H), 1.82 (s, 1H), |
| | | | | | | 1.57 (d, J = 13.3 |
| | | | | | | Hz, 3H), 1.45- |
| | | | | | | 1.13 (m, 10H), |
| | | | | | | 0.90-0.59 (m, |
| | | | | | | 5H). |
| 46 | A | | A | 472.64 | 3.8 | 1H NMR (300 |
| | | | | | | MHz, CDCl3) δ |
| | | | | | | 6.88 (s, 1H), 5.20 |
| | | | | | | (d, J = 16.3 Hz, |
| | | | | | | 1H), 3.86 (d, J = |
| | | | | | | 16.3 Hz, 1H), |
| | | | | | | 3.72-3.22 (m, |
| | | | | | | 4H), 2.34 (t, J = |
| | | | | | | 11.6 Hz, 1H), |
| | | | | | | 1.90 (d, J = 13.4 |
| | | | | | | Hz, 1H), 1.76- |
| | | | | | | 1.47 (m, 10H), |
| | | | | | | 1.30 (d, J = 19.2 |
| | | | | | | Hz, 11H), 0.82 |
| | | | | | | (d, J = 6.5 Hz, |
| | | | | | | 5H). |
| 47 | A | | A | 503.6 | 3.95 | |
| 48 | A | | B | 517.6 | 3.96 | 1H NMR (300 |
| | | | | | | MHz, CDCl3) δ |
| | | | | | | 6.70 (s 1H), 5.15- |
| | | | | | | 4.89 (m, 1H), |
| | | | | | | 4.54-4.23(m, |
| | | | | | | 2H), 4.05-3.44 |
| | | | | | | (m, 4H), 3.22- |
| | | | | | | 2.82 (m, 2H), |
| | | | | | | 2.67-2.22 (m, |
| | | | | | | 2H), 2.21-0.96 |
| | | | | | | (m, 22H), 0.88- |
| | | | | | | 0.59 (m, 6H). |
| 49 | A | | A | 503.6 | 3.36 | 1H NMR (300 |
| | | | | | | MHz, CDCl3) δ |
| | | | | | | 6.71 (s, 1H), 5.15- |
| | | | | | | 4.88 (m, 2H), |
| | | | | | | 4.58-4.25 (m, |
| | | | | | | 2H), 4.10-3.44 |
| | | | | | | (m, 5H), 2.12- |
| | | | | | | 1.95 (m, 2H), |
| | | | | | | 1.88-0.92 (m, |
| | | | | | | 21H), 0.79-0.54 |
| | | | | | | (m, 5H). |
| 50 | A | | A | 517.6 | 3.97 | 1H NMR (300 |
| | | | | | | MHz, CDCl3) δ |
| | | | | | | 6.70 (s, 1H), |
| | | | | | | 5.14-4.88 (m, 1H), |
| | | | | | | 4.18-3.95 (m, |
| | | | | | | 1H), 3.90-3.08 |
| | | | | | | (m, 6H), 2.21- |
| | | | | | | 0.88 (m, 28H), |
| | | | | | | 0.82-0.62 (m, |
| | | | | | | 6H). |
| 51 | A | | A | 517.6 | 3.3 | 1H NMR (300 |
| | | | | | | MHz, CDCl3) δ |
| | | | | | | 6.73 (s, 1H), |
| | | | | | | 4.88-4.51 (m, |
| | | | | | | 2H), 4.25- |
| | | | | | | 3.95(m, 3H), |
| | | | | | | 3.65-3.42 |
| | | | | | | (m, 3H), 3.17- |
| | | | | | | 3.02 (m, 2H), |
| | | | | | | 2.97-2.80 (m, |
| | | | | | | 3H), 2.21-0.86 |
| | | | | | | (m, 22H), 0.82- |
| | | | | | | .61 (m, 4H). |
| 52 | A | | A | 489.6 | 3.65 | 1H NMR (300 |
| | | | | | | MHz, CDCl3) δ |
| | | | | | | 6.70 (s, 1H), 4.98- |
| | | | | | | 3.83 (m, 8H), |
| | | | | | | 3.42-3.27 (m, |
| | | | | | | 3H), 2.84 (s, 1H), |
| | | | | | | 2.11-1.95 (m, |
| | | | | | | 2H), 1.87-1.40 |
| | | | | | | (m, 5H), 1.39- |
| | | | | | | 0.91 (m, 12H), |
| | | | | | | 0.86-0.67 (m, |
| | | | | | | 4H). |
| 53 | A | | A | 517.6 | 3.7 | |
| 54 | A | | A | 503.6 | 2.79 | |
| 55 | C | | A | 491.39 | 2.85 | ¹H (300 MHz, d6- |
| | | | | | | DMSO) δ 13.58 |
| | | | | | | (br s, 1H), 7.21 |
| | | | | | | (d, 1H), 4.92 (dd, |
| | | | | | | 1H), 3.85 (dd, |
| | | | | | | 1H), 3.55-3.35 |
| | | | | | | (m, 8H), 2.75- |
| | | | | | | 2.60 (m, 1H), |
| | | | | | | 2.30-2.20 (m, |
| | | | | | | 1H), 1.77-0.38 |
| | | | | | | (m, 19H); 23% of |
| | | | | | | the epimeric |
| | | | | | | alcohol is also |
| | | | | | | present in this |
| | | | | | | diastereomeric |
| | | | | | | mixture: δ 13.58 |
| | | | | | | (br s, 1H), 7.19 |
| | | | | | | (d, 1H), 4.92 (dd, |
| | | | | | | 1H), 3.82 (dd, |
| | | | | | | 1H), 3.55-3.35 |
| | | | | | | (m, 8H), 2.75- |
| | | | | | | 2.60 (m, 1H), |
| | | | | | | 2.30-2.20 (m, |
| | | | | | | 1H), 1.77-0.38 |
| | | | | | | (m, 19H). |
| 56 | A | A | B | 517.36 | 5.54 | 1H NMR (300 |
| | | | | | | MHz, DMSO) δ |
| | | | | | | 13.55 (br, 1H), |
| | | | | | | 7.44-6.97 (m, |
| | | | | | | 1H), 5.80-5.06 |
| | | | | | | (m, 1H), 4.36- |
| | | | | | | 3.08 (m, 8H), |
| | | | | | | 2.15-0.95 (m, |
| | | | | | | 20H), 0.89-0.45 |
| | | | | | | (m, 9H). |
| 57 | A | | A | 490.97 | 3.75 | |
| 58 | A | | A | 533.63 | 4.16 | |
| 59 | A | | A | 477.9 | 2.51 | |
| 60 | A | | A | 519.58 | 2.8 | |
| 61 | A | A | A | 533.36 | 1.47 | 1H NMR (300 |
| | | | | | | MHz, CDCl3) δ |
| | | | | | | 7.50 (s, 0.4H), |
| | | | | | | 6.76 (s, 0.6H), |
| | | | | | | 5.52 (dd, J = 9.4, |
| | | | | | | 5.7 Hz, 0.4H), |
| | | | | | | 5.27 (dd, J = |
| | | | | | | 10.7, 4.9 Hz, |
| | | | | | | 0.6H), 4.09- |
| | | | | | | 3.54 (m, 11H), |
| | | | | | | 3.42-3.18 (m, |
| | | | | | | 3H), 2.14-1.22 |
| | | | | | | (m, 20H), 0.91- |
| | | | | | | 0.59 (m, 4H). |
| 62 | A | | A | 460 | 1.92 | (DMSO, |
| | | | | | | 300 MHz) 0.55- |
| | | | | | | 0.72 (2H, m), |
| | | | | | | 0.80 (3H, d), |
| | | | | | | 1.20 (6H, d), |
| | | | | | | 1.20-1.45 (2H, |
| | | | | | | m), 1.50-1.70 |
| | | | | | | (4H, m), 2.15 |
| | | | | | | (1H, t), 2.82 (1H, |
| | | | | | | sept), 3.45 (4H, |
| | | | | | | app s), 3.55 (4H, |
| | | | | | | app s), 3.80 (1H, |
| | | | | | | d), 4.89 (1H, d), |
| | | | | | | 7.24 (1H, s), |
| | | | | | | 13.50 (1H, br s). |
| 63 | B | | B | 517.36 | 1.81 | 1H NMR (300 |
| | | | | | | MHz, CDCl3) δ |
| | | | | | | 7.64 (s, 0.4H), |
| | | | | | | 6.85 (s, 0.6H), |
| | | | | | | 5.36 (dd, 0.4H), |
| | | | | | | 5.02 (t, 0.6H), |
| | | | | | | 4.00-3.15 (m, |
| | | | | | | 10H), 2.13-0.98 |
| | | | | | | (m, 24H), 0.96- |
| | | | | | | 0.43 (m, 4H) |
| 64 | A | A | B | 517.36 | 1.9 | 1H NMR (300 |
| | | | | | | MHz, CDCl3) δ |
| | | | | | | 7.68 (s, 0.5H), |
| | | | | | | 6.65 (s, 0.5H), |
| | | | | | | 5.30-5.15 (m, |
| | | | | | | 1H), 3.80-3.35 |
| | | | | | | (m, 9H), 2.00- |
| | | | | | | 0.50 (m, 29H) |
| 65 | A | | A | 532.99 | 3.87 | 1H NMR (300 |
| | | | | | | MHz, CDCl3) δ |
| | | | | | | 7.95 (d, J = 3.5 |
| | | | | | | Hz, 0.1H) 6.80 (d, |
| | | | | | | J = 3.1 Hz, |
| | | | | | | 0.9H), 5.57- |
| | | | | | | 5.23 (m, 1H), |
| | | | | | | 4.01-3.55 (m, |
| | | | | | | 9H), 3.54-3.23 |
| | | | | | | (m, 3H), 3.14 (m, |
| | | | | | | 4H), 2.04 (m, 1H), |
| | | | | | | 1.67 1.58 (m, |
| | | | | | | 4H), 1.45-1.19 |
| | | | | | | (m, 12H), 1.11- |
| | | | | | | 0.96 (m, 1H), |
| | | | | | | 0.91-0.47 (m, |
| | | | | | | 4H). |
| 66 | A | | A | 490.97 | 3.9 | |
| 67 | A | | A | 419.44 | 3.47 | (400 MHz, |
| | | | | | | DMSO) 0.50- |
| | | | | | | 0.85 (3H, m), |
| | | | | | | 0.80 (3H, d), |
| | | | | | | 1.19 (6H, d), |
| | | | | | | 1.20-1.69 (6H, |
| | | | | | | m), 2.09 (1H, |
| | | | | | | quin), 2.80 (3H, |
| | | | | | | s), 2.87 (1H, |
| | | | | | | sept), 2.92 (3H, |
| | | | | | | s), 3.85 (1H, d), |
| | | | | | | 4.86 (1H, d), |
| | | | | | | 7.22 (1H, s), |
| | | | | | | 13.59 (1H, br s). |
| 68 | A | | B | 475.33 | 1.84 | (1H NMR (300 |
| | | | | | | MHz, CDCl3) δ |
| | | | | | | 7.60 (s, 0.4H), |
| | | | | | | 6.74 (s, 0.6H), |
| | | | | | | 5.32 (dd, 0.4H), |
| | | | | | | 5.07 (dd, 0.6H), |
| | | | | | | 3.39 (s, 3H), 3.10- |
| | | | | | | 2.94 (m, 4H), |
| | | | | | | 2.14-1.22 (m, |
| | | | | | | 29H). |
| 69 | A | | A | 460.24 | 2.54 | (DMSO, |
| | | | | | | 400 MHz) 0.55- |
| | | | | | | 0.90 (3H, m), |
| | | | | | | 0.81 (3H. d), |
| | | | | | | 1.20 (6H, d), |
| | | | | | | 1.20-1.39 (2H, |
| | | | | | | m), 1.50-1.68 |
| | | | | | | (4H, m), 2.14 |
| | | | | | | (1H, t), 2.90 (1H, |
| | | | | | | sept), 3.10 (4H, |
| | | | | | | br s), 3.59 (4H, |
| | | | | | | br s), 3.88 (1H, |
| | | | | | | d), 5.00 (1H, d), |
| | | | | | | 7.20 (1H, s), 8.90 |
| | | | | | | (1H, br s), 13.58 |
| | | | | | | (1H, br s). |
| 70 | A | | A | 405.55 | 3.27 | ¹H (300 MHz, d6- |
| | | | | | | DMSO) δ 13.80 |
| | | | | | | (br s, 1H), 7.88 |
| | | | | | | (br s, 1H), 7.30 |
| | | | | | | (s, 1H), 4.54 (d, |
| | | | | | | 1H), 3.73 (d, 1H), |
| | | | | | | 2.92 (sept, 1H), |
| | | | | | | 2.58 (d, 3H), |
| | | | | | | 2.08 (t, 1H), 1.67- |
| | | | | | | 1.51 (m, 4H), |
| | | | | | | 1.52-1.25 (m, |
| | | | | | | 4H), 1.25 (d, 6H), |
| | | | | | | 0.85 (d, 3H), |
| | | | | | | 0.91-0.50 (m, |
| | | | | | | 1H). |
| 71 | A | | A | 473.37 | 3.19 | ¹H (300 MHz, d6- |
| | | | | | | DMSO) δ 13.53 |
| | | | | | | (br s, 1H), 7.22 |
| | | | | | | (d, 1H), 4.68 (d, |
| | | | | | | 4H), 4.33 (t, 1H), |
| | | | | | | 4.30 (t, 1H), 4.06- |
| | | | | | | 4.00 (m, 2H), |
| | | | | | | 3.59-3.55 (m, |
| | | | | | | 2H), 2.82 (sept, |
| | | | | | | 1H), 2.08 (t, 1H), |
| | | | | | | 1.70-1.24 (m, |
| | | | | | | 7H), 1.24 (d, 6H), |
| | | | | | | 0.80 (d, 3H), |
| | | | | | | 0.90-0.56 (m, |
| | | | | | | 2H). |
| 72 | A | | A | 532.99 | 3.96 | |
| 73 | A | | A | 447.43 | 1.55 | |
| 74 | A | | A | 533 | 1.66 | |
| 75 | A | | B | 491 | 1.85 | |
| 76 | A | | B | 544.04 | 2.12 | |
| 77 | A | | A | 491 | 3.96 | |
| 78 | A | A | A | 515 | 1.45 | |
| 79 | A | A | A | 503.6 | 1.68 | |
| 80 | A | | A | 531.7 | 1.8 | |
| 81 | A | | B | 531.6 | 1.8 | |
| 82 | A | A | A | 516 | 2.12 | |
| 83 | A | A | A | 445 | 1.24 | 1H NMR (300 |
| | | | | | | MHz, CDCl3) δ |
| | | | | | | 6.94 (s, 0.5H), |
| | | | | | | 6.91 (s, 0.5H), |
| | | | | | | 5.15 (t, J = 9.5 |
| | | | | | | Hz, 0.5H), 4.34 |
| | | | | | | (t, J = 9.4 Hz, |
| | | | | | | 0.5H), 3.84- |
| | | | | | | 3.32 (m, 2H), |
| | | | | | | 2.94 (s, 1.5H), |
| | | | | | | 2.88 (s, 1.5H), |
| | | | | | | 2.60-2.26 (m, |
| | | | | | | 1H), 2.23-1.97 |
| | | | | | | (m, 1H), 1.94- |
| | | | | | | 1.16 (m, 17H), |
| | | | | | | 0.95-0.57 (m, |
| | | | | | | 5H). |
| 84 | A | | B | 517 | 3.85 | 1H NMR (300 |
| | | | | | | MHz, CDCl3) δ |
| | | | | | | 6.75 (s, 1H), 4.92 |
| | | | | | | (m, 1H), 3.31 (s, |
| | | | | | | 3H), 3.04 (d, J = |
| | | | | | | 16.6 Hz, 3H), |
| | | | | | | 2.83 (m, 2H), |
| | | | | | | 2.11-1.91 (m, |
| | | | | | | 3H), 1.84-1.37 |
| | | | | | | (m, 9H), 1.36- |
| | | | | | | 1.20 (m, 8H), |
| | | | | | | 1.05-0.54 (m, |
| | | | | | | 10H). |
| 85 | A | | B | 517 | 3.79 | |
| 86 | A | | B | 475 | 3.65 | |
| 87 | A | | A | 532.9 | 3.6 | |
| 88 | A | | A | 491 | 3.6 | 1H NMR (300 |
| | | | | | | MHz, CDCl3) δ |
| | | | | | | 6.91 (s, 1H), |
| | | | | | | 5.18-5.12 (m, |
| | | | | | | 1H), 3.54-3.35 |
| | | | | | | (m, 2H), 3.34- |
| | | | | | | 3.23 (m, 3H), |
| | | | | | | 3.13 (m, 4H), |
| | | | | | | 2.03-1.55 (m, |
| | | | | | | 21H), 1.12 (m, |
| | | | | | | 2H), 0.98-0.60 |
| | | | | | | (m, 4H). |
| 89 | A | | A | 447 | 3.8 | 1H NMR (300 |
| | | | | | | MHz, CDCl3) δ |
| | | | | | | 6.75 (s, 1H), 4.92 |
| | | | | | | (m, 1H), 3.31 (s, |
| | | | | | | 3H), 3.07 (s, 3H), |
| | | | | | | 3.01 (s, 1H), 2.83 |
| | | | | | | (m, 2H), 2.12- |
| | | | | | | 1.91 (m, 2H), |
| | | | | | | 1.85-1.37 (m, |
| | | | | | | 7H), 1.37-1.20 |
| | | | | | | (m, 6H), 1.09- |
| | | | | | | 0.48 (m, 8H). |
| 90 | A | | A | 489 | 3.7 | 1H NMR (300 |
| | | | | | | MHz, CDCl3) δ |
| | | | | | | 6.78 (s, 1H), 5.20 |
| | | | | | | (m, 2H), 4.02- |
| | | | | | | 3.28 (m, 2H), |
| | | | | | | 2.83 (m, 1H), |
| | | | | | | 2.53-2.27 (m, |
| | | | | | | 3H), 2.01 (m, |
| | | | | | | 2H), 1.91-1.19 |
| | | | | | | (m, 15H), 1.08- |
| | | | | | | 0.56 (m, 10H). |
| 91 | A | A | A | 445.19 | 4.79 | 1H NMR (300 |
| | | | | | | MHz, CDCl3) δ |
| | | | | | | 6.94 (s, 0.5H), |
| | | | | | | 6.91 (s, 0.5H), |
| | | | | | | 5.20 (t, 0.5H), |
| | | | | | | 4.34 (t, 0.5H), |
| | | | | | | 3.64-3.35 (m, |
| | | | | | | 2H), 2.90 (s, |
| | | | | | | 1.5H), 2.80 (s, |
| | | | | | | 1.5H), 2.50- |
| | | | | | | 2.26 (m, 1H), |
| | | | | | | 2.23-2.02 (m, |
| | | | | | | 1H), 1.84-1.26 |
| | | | | | | (m, 17H), 0.80- |
| | | | | | | 0.65 (m, 5H). |
| 92 | A | | A | 445.25 | 4.79 | 1H NMR (300 |
| | | | | | | MHz, CDCl3) δ |
| | | | | | | 6.92 (s, 0.5H), |
| | | | | | | 6.90 (s, 0.5H), |
| | | | | | | 5.21 (t, 0.5H), |
| | | | | | | 4.35 (t, 0.5H), |
| | | | | | | 3.66-3.37 (m, |
| | | | | | | 2H), 2.92 (s, |
| | | | | | | 1.5H), 2.82 (s, |
| | | | | | | 1.5H), 2.53- |
| | | | | | | 2.24 (m, 1H), |
| | | | | | | 2.26-2.02 (m, |
| | | | | | | 1H), 1.84-1.26 |
| | | | | | | (m, 17H), 0.80- |
| | | | | | | 0.65 (m, 5H). |
| 93 | A | | A | 515 | 2.6 | |
| 94 | A | A | A | 514.98 | 3.1 | 1H NMR (300 |
| | | | | | | MHz, CDCl3) d |
| | | | | | | 6.98 (s, 1H), |
| | | | | | | 4.92-4.15 (m, |
| | | | | | | 5H), 3.65-2.73 |
| | | | | | | (br, 10H), 2.35- |
| | | | | | | 2.21 (m, 1H), |
| | | | | | | 1.73-1.50 (m, |
| | | | | | | 5H), 1.32 (s, 9H), |
| | | | | | | 1.33-1.22 (m, |
| | | | | | | 2H), 0.85 (d, 3H), |
| | | | | | | 0.80-0.60 (m, |
| | | | | | | 2H). |
| 95 | A | A | B | 473.21 | 1.56 | 1H NMR (300 |
| | | | | | | MHz, CDCl3) d |
| | | | | | | 6.74 (s, 1H), 5.22 |
| | | | | | | (br s, 1H), 5.10 |
| | | | | | | (d, J = 10.4 Hz, |
| | | | | | | 1H), 3.99-3.61 |
| | | | | | | (m, 2H), 3.27 (d, |
| | | | | | | J = 15.3 Hz, 2H), |
| | | | | | | 3.07 (2 × s, 3H), |
| | | | | | | 2.20-1.31 (m, |
| | | | | | | 12H), 1.34 (2 × s, |
| | | | | | | 9H), 0.87-0.57 |
| | | | | | | (m, 5H). |
| 96 | A | | B | 461.4 | 5.31 | 1H NMR (300 |
| | | | | | | MHz, DMSO) d |
| | | | | | | 13.53 (s, 1H), |
| | | | | | | 7.54 (s, 0.65H), |
| | | | | | | 7.32 (s, 0.35H), |
| | | | | | | 4.98 (d, J = 6.8 |
| | | | | | | Hz, 0.65H), 4.60 |
| | | | | | | (s, 0.35H), 3.60 |
| | | | | | | (s, 0.65H), 3.34 |
| | | | | | | (s, 1.35H), 3.10- |
| | | | | | | 2.70 (m, 5H), |
| | | | | | | 2.63-2.34 (m, |
| | | | | | | 2H), 2.10 (dd, |
| | | | | | | J = 15.0, 9.0 Hz, |
| | | | | | | 0.35H), 1.93- |
| | | | | | | 1.71 (m, 1.65H), |
| | | | | | | 1.64-1.05 (m, |
| | | | | | | 14H), 0.94-0.44 |
| | | | | | | (m, 6H). |
| 97 | A | | A | 489.47 | 5.3 | |
| 98 | A | B | A | 447.43 | 5.22 | 1H NMR (300 |
| | | | | | | MHz, CDCl3) δ |
| | | | | | | 6.82 (s, 1H), 3.94- |
| | | | | | | 3.66 (m, 2H), |
| | | | | | | 3.04 (s, 3H), 2.82 |
| | | | | | | (d, J = 18.7 Hz, |
| | | | | | | 3H), 2.73-2.46 |
| | | | | | | (m, 2H), 2.00 (t, |
| | | | | | | J = 11.7 Hz, 1H), |
| | | | | | | 1.65-1.11 (m, |
| | | | | | | 15H), 0.73 (d, |
| | | | | | | J = 6.5 Hz, 5H). |
| 99 | A | | B | 459.59 | 2.02 | 1H NMR (300 |
| | | | | | | MHz, DMSO) δ |
| | | | | | | 13.5 (br, 1H), |
| | | | | | | 7.60 (s, 0.6H), |
| | | | | | | 7.35 (s, 0.4H), |
| | | | | | | 5.01-4.95 (m, |
| | | | | | | 0.6H), 4.65- |
| | | | | | | 4.49 (m, 0.4H), |
| | | | | | | 3.65 (br, 0.4H), |
| | | | | | | 3.25 (br, 0.6H), |
| | | | | | | 3.00 (s, 1.8H), |
| | | | | | | 3.85 (s, 1.2H), |
| | | | | | | 2.80 (s, 1.2H), |
| | | | | | | 2.75 (s, 1.8H), |
| | | | | | | 2.60-1.75 (m, |
| | | | | | | 2H), 1.60-1.12 |
| | | | | | | (m, 8H), 1.30 (s, |
| | | | | | | 9H), 0.85 (d, 3H), |
| | | | | | | 0.75 (d, 3H), |
| | | | | | | 0.65-0.50 (m, |
| | | | | | | 1H) |
| 100 | A | | A | 461.38 | 1.37 | 1H NMR (300 |
| | | | | | | MHz, CDCl3) δ |
| | | | | | | 7.30 (s, 0.4H), |
| | | | | | | 6.85 (s, 0.6H), |
| | | | | | | 5.03-4.85 (m, |
| | | | | | | 1H), 3.27-2.90 |
| | | | | | | (m, 6H), 2.75 (br, |
| | | | | | | 1H), 2.25-1.80 |
| | | | | | | (m, 3H), 1.75- |
| | | | | | | 1.50 (m, 4H), |
| | | | | | | 1.30-1.12 (m, |
| | | | | | | 6H), 1.35 (s, 9H), |
| | | | | | | 0.75 (d, 3H), |
| | | | | | | 0.65-0.50 (m, |
| | | | | | | 1H) |
| 101 | A | | B | 487 | 1.51 | 1H NMR (300 |
| | | | | | | MHz, DMSO) d |
| | | | | | | 7.44 (s, 0.6H), |
| | | | | | | 7.32 (s, 0.4H), |
| | | | | | | 4.96 (dd, J = |
| | | | | | | 14.1, 7.0 Hz, |
| | | | | | | 0.6H), 4.61- |
| | | | | | | 4.48 (m, 0.4H), |
| | | | | | | 3.70-3.12 (m, |
| | | | | | | 6H), 2.85 (dd, |
| | | | | | | J = 15.4, 5.7 Hz, |
| | | | | | | 0.4H), 2.48- |
| | | | | | | 2.32 (m, 1H), |
| | | | | | | 2.09 (dd, J = |
| | | | | | | 15.3, 8.7 Hz, |
| | | | | | | 0.6H), 1.79 (ddt, |
| | | | | | | J = 12.7, 9.7, 6.6 |
| | | | | | | Hz, 5H), 1.62- |
| | | | | | | 1.04 (m, 16H), |
| | | | | | | 0.90 (d, J = 6.8 |
| | | | | | | Hz, 2H), 0.76 (d, |
| | | | | | | J = 6.5 Hz, 2H), |
| | | | | | | 0.60 (dt, J = |
| | | | | | | 34.5, 11.4 Hz, |
| | | | | | | 2H). |
| 102 | A | | B | 487 | 1.51 | 1H NMR (300 |
| | | | | | | MHz, DMSO) d |
| | | | | | | 7.44 (s, 0.6H), |
| | | | | | | 7.32 (s, 0.4H), |
| | | | | | | 4.96 (dd, J = |
| | | | | | | 14.1, 7.0 Hz, |
| | | | | | | 0.6H), 4.61- |
| | | | | | | 4.48 (m, 0.4H), |
| | | | | | | 3.70-3.12 (m, |
| | | | | | | 6H), 2.85 (dd, |
| | | | | | | J = 15.4, 5.7 Hz, |
| | | | | | | 0.4H), 2.48- |
| | | | | | | 2.32 (m, 1H), |
| | | | | | | 2.09 (dd, J = |
| | | | | | | 15.3, 8.7 Hz, |
| | | | | | | 0.6H), 1.79 (ddt, |
| | | | | | | J = 12.7, 9.7, 6.6 |
| | | | | | | Hz, 5H), 1.62- |
| | | | | | | 1.04 (m, 16H), |
| | | | | | | 0.90 (d, J = 6.8 |
| | | | | | | Hz, 2H), 0.76 (d, |
| | | | | | | J = 6.5 Hz, 2H), |
| | | | | | | 0.60 (dt, J = |
| | | | | | | 34.5, 11.4 Hz, |
| | | | | | | 2H). |
| 103 | A | | A | 487 | 1.51 | 1H NMR (300 |
| | | | | | | MHz, DMSO) d |
| | | | | | | 7.44 (s, 0.6H), |
| | | | | | | 7.32 (s, 0.4H), |
| | | | | | | 4.96 (dd, J = |
| | | | | | | 14.1, 7.0 Hz, |
| | | | | | | 0.6H), 4.61- |
| | | | | | | 4.48 (m, 0.4H), |
| | | | | | | 3.70-3.12 (m, |
| | | | | | | 6H), 2.85 (dd, |
| | | | | | | J = 15.4, 5.7 Hz, |
| | | | | | | 0.4H), 2.48- |
| | | | | | | 2.32 (m, 1H), |
| | | | | | | 2.09 (dd, J = |
| | | | | | | 15.3, 8.7 Hz, |
| | | | | | | 0.6H), 1.79 (ddt, |
| | | | | | | J = 12.7, 9.7, 6.6 |
| | | | | | | Hz, 5H), 1.62- |
| | | | | | | 1.04 (m, 16H), |
| | | | | | | 0.90 (d, J = 6.8 |
| | | | | | | Hz, 2H), 0.76 (d, |
| | | | | | | J = 6.5 Hz, 2H), |
| | | | | | | 0.60 (dt, J = |
| | | | | | | 34.5, 11.4 Hz, |
| | | | | | | 2H). |
| 104 | A | | A | 503.35 | 3.63 | 1H NMR (300 |
| | | | | | | MHz, CDCl3) d |
| | | | | | | 6.94 (s, 0.5H), |
| | | | | | | 6.80 (s, 0.5H), |
| | | | | | | 4.07-3.95 (m, |
| | | | | | | 1H), 3.75-3.33 |
| | | | | | | (m, 10H), 2.15- |
| | | | | | | 2.01 (m, 1H), |
| | | | | | | 1.73-1.40 (m, |
| | | | | | | 7H), 1.32 (s, 9H), |
| | | | | | | 1.13-1.02 (m, |
| | | | | | | 3H), 0.80 (d, 3H), |
| | | | | | | 0.80-0.60 (m, |
| | | | | | | 2H). |
| 105 | A | | A | 503 | 1.36 | 1H NMR (300 |
| | | | | | | MHz, DMSO) d |
| | | | | | | 7.54 (s, 0.66H), |
| | | | | | | 7.29 (s, 0.33H), |
| | | | | | | 4.99 (dd, J = |
| | | | | | | 14.3, 7.1 Hz, |
| | | | | | | 0.66H), 4.63 (s, |
| | | | | | | 0.33H), 3.85- |
| | | | | | | 3.14 (m, 9H), |
| | | | | | | 2.75-2.10 (m, |
| | | | | | | 5H), 1.76 (s, 1H), |
| | | | | | | 1.64-1.05 (m, |
| | | | | | | 14H), 0.81 (dd, |
| | | | | | | J = 32.2, 6.6 Hz, |
| | | | | | | 6H). |
| 106 | A | | A | 503 | 1.36 | 1H NMR (300 |
| | | | | | | MHz, DMSO) d |
| | | | | | | 7.53 (s, 0.66H), |
| | | | | | | 7.28 (s, 0.33H), |
| | | | | | | 4.99 (dd, J = |
| | | | | | | 14.0, 6.9 Hz, |
| | | | | | | 0.66H), 4.63 (s, |
| | | | | | | 0.33H), 3.79- |
| | | | | | | 3.15 (m, 9H), |
| | | | | | | 2.85-2.10 (m, |
| | | | | | | 5H), 1.83 (t, J = |
| | | | | | | 11.7 Hz, 1H), |
| | | | | | | 1.62-1.02 (m, |
| | | | | | | 14H), 0.81 (dd, |
| | | | | | | J = 32.3, 6.6 Hz, |
| | | | | | | 6H). |
| 107 | A | | A | 445.25 | 3.51 | 1H NMR (300 |
| | | | | | | MHz, CDCl3) d |
| | | | | | | 6.84 (s, 0.5H), |
| | | | | | | 6.80 (s, 0.5H), |
| | | | | | | 5.57-5.34 (m, |
| | | | | | | 0.5H), 4.35- |
| | | | | | | 4.23 (m, 0.5H), |
| | | | | | | 3.85-3.61 (m, |
| | | | | | | 1H), 3.33-3.00 |
| | | | | | | (m, 1H), 3.85- |
| | | | | | | 3.60 (m, 6H), |
| | | | | | | 2.15-1.85 (m, |
| | | | | | | 1H), 1.70-1.45 |
| | | | | | | (m, 7H), 1.32 (s, |
| | | | | | | 9H), 0.80-0.60 |
| | | | | | | (m, 5H). |
| 108 | A | A | A | 503.35 | 1.35 | 1H NMR (300 |
| | | | | | | MHz, CDCl3) d |
| | | | | | | 6.94 (s, 0.5H), |
| | | | | | | 6.80 (s, 0.5H), |
| | | | | | | 4.07-3.95 (m, |
| | | | | | | 1H), 3.75-3.33 |
| | | | | | | (m, 10H), 2.15- |
| | | | | | | 2.01 (m, 1H), |
| | | | | | | 1.73-1.40 (m, |
| | | | | | | 7H), 1.32 (s, 9H), |
| | | | | | | 1.13-1.02 (m, |
| | | | | | | 3H), 0.80 (d, 3H), |
| | | | | | | 0.80-0.60 (m, |
| | | | | | | 2H). |
| 109 | A | | A | 503.35 | 1.35 | 1H NMR (300 |
| | | | | | | MHz, CDCl3) d |
| | | | | | | 6.94 (s, 0.5H), |
| | | | | | | 6.80 (s, 0.5H), |
| | | | | | | 4.07-3.95 (m, |
| | | | | | | 1H), 3.75-3.33 |
| | | | | | | (m, 10H), 2.15- |
| | | | | | | 2.01 (m, 1H), |
| | | | | | | 1.73-1.40 (m, |
| | | | | | | 7H), 1.32 (s, 9H), |
| | | | | | | 1.13-1.02 (m, |
| | | | | | | 3H), 0.80 (d, 3H), |
| | | | | | | 0.80-0.60 (m, |
| | | | | | | 2H). |
| 110 | A | | A | 516 | 0.58 | 1H NMR (300 |
| | | | | | | MHz, DMSO) d |
| | | | | | | 7.55 (s, 0.65H), |
| | | | | | | 7.30 (s, 0.35H), |
| | | | | | | 4.98 (dd, J = |
| | | | | | | 13.9, 6.8 Hz, |
| | | | | | | 0.65H), 4.64 (s, |
| | | | | | | 0.35H), 3.65- |
| | | | | | | 2.95 (m, 7H ), |
| | | | | | | 2.80-2.59 (m, |
| | | | | | | 3H), 2.50-2.20 |
| | | | | | | (m, 7H), 1.95- |
| | | | | | | 1.06 (m, 15H), |
| | | | | | | 0.81 (dd, J = |
| | | | | | | 30.9, 6.6 Hz, |
| | | | | | | 6H). |
| 111 | A | | A | 461 | 1.41 | 1H NMR (300 |
| | | | | | | MHz, DMSO) d |
| | | | | | | 7.31 (d, J = 23.9 |
| | | | | | | Hz, 1H), 5.01 (d, |
| | | | | | | J = 8.6 Hz, |
| | | | | | | 0.65H), 4.69 (s, |
| | | | | | | 0.35H), 3.60 (s, |
| | | | | | | 0.65H), 3.34 (s, |
| | | | | | | 1.35H), 3.02 (d, |
| | | | | | | J = 7.2 Hz, 2H), |
| | | | | | | 2.23 (ddd, J = |
| | | | | | | 40.9, 20.1, 12.4 |
| | | | | | | Hz, 1H), 1.96- |
| | | | | | | 0.44 (m, 27H). |
| 112 | A | | A | 459.5 | 2.8 | |
| 113 | A | | A | 544 | 0.58 | 1H NMR (300 |
| | | | | | | MHz, DMSO) d |
| | | | | | | 7.51 (s, 0.65H), |
| | | | | | | 7.34 (s, 0.35H), |
| | | | | | | 5.00 (d, J = 6.7 |
| | | | | | | Hz, 0.65H), 4.50 |
| | | | | | | (dd, J = 39.4, |
| | | | | | | 27.1 Hz, 1.35H), |
| | | | | | | 3.55-2.30 (m, |
| | | | | | | 16H), 2.23-1.03 |
| | | | | | | (m, 19H), 0.82 |
| | | | | | | (dd, J = 36.1, 6.6 |
| | | | | | | Hz, 6H). |
| 114 | A | | A | 505 | 1.47 | 1H NMR (300 |
| | | | | | | MHz, DMSO) d |
| | | | | | | 7.52-7.25 (m, |
| | | | | | | 1H), 5.06-4.81 |
| | | | | | | (m, 0.65H), 4.57 |
| | | | | | | (s, 0.35H), 3.59- |
| | | | | | | 3.12 (m, 8H), |
| | | | | | | 3.10-2.80 (m, |
| | | | | | | 2H), 2.78 (d, J = |
| | | | | | | 5.5 Hz, 1H), |
| | | | | | | 2.60-2.00 (m, 5H), |
| | | | | | | 1.97-0.97 (m, |
| | | | | | | 15H), 0.96-0.42 |
| | | | | | | (m, 6H). |
| 115 | A | | A | 531 | 1.54 | 1H NMR (300 |
| | | | | | | MHz, DMSO) d |
| | | | | | | 7.56-7.25 (m, |
| | | | | | | 1H), 4.98 (d, J = |
| | | | | | | 4.5 Hz, 0.65H), |
| | | | | | | 4.60 (s, 0.35H), |
| | | | | | | 3.80-3.60 (m, |
| | | | | | | 2H), 3.46-2.80 |
| | | | | | | (m, 7H), 2.68- |
| | | | | | | 2.22 (m, 5H), |
| | | | | | | 2.18-0.41 (m, |
| | | | | | | 25H). |
| 116 | A | | A | 503 | 1.32 | 1H NMR (300 |
| | | | | | | MHz, DMSO) δ |
| | | | | | | 7.40-7.21 (m, |
| | | | | | | 1H), 4.96-4.76 |
| | | | | | | (m, 0.66H), |
| | | | | | | 4.51-3.74 (m, |
| | | | | | | 4.33H), 3.58 (s, |
| | | | | | | 1H), 3.23-3.14 |
| | | | | | | (m, 3H), 2.78- |
| | | | | | | 2.25 (m, 1H), |
| | | | | | | 2.28 (d, J = 6.9 |
| | | | | | | Hz, 1H), 2.00- |
| | | | | | | 0.46 (m, 22H). |
| 117 | A | | A | 459.5 | 1.57 | 1H NMR (300 |
| | | | | | | MHz, CDCl3) δ |
| | | | | | | 6.83 (d, J = 9.7 |
| | | | | | | Hz, 1H), 5.32 (s, |
| | | | | | | 1H), 3.82-3.51 |
| | | | | | | (m, 2H), 3.28- |
| | | | | | | 3.22 (m, 1H), |
| | | | | | | 3.07 (m, 3H), |
| | | | | | | 2.65-2.37 (m, |
| | | | | | | 1H), 2.22-2.18 |
| | | | | | | (m, 3H), 1.89- |
| | | | | | | 1.38 (m, 9H), |
| | | | | | | 1.35 (s, 9H), 0.94- |
| | | | | | | 0.76 (m, 4H). |
| 118 | A | | | 490.23 | 2.16 | 1H NMR (300 |
| | | | | | | MHz, CDCl3) d |
| | | | | | | 6.98 (s, 1H), |
| | | | | | | 4.92-4.15 (m, |
| | | | | | | 5H), 3.65-2.73 |
| | | | | | | (br, 10H), 2.35- |
| | | | | | | 2.21 (m, 1H), |
| | | | | | | 1.73-1.50 (m, |
| | | | | | | 5H), 1.32 (s, 9H), |
| | | | | | | 1.33-1.22 (m, |
| | | | | | | 2H), 0.85 (d, 3H), |
| | | | | | | 0.80-0.60 (m, |
| | | | | | | 2H). |
| 119 | A | | | 488.25 | 2.17 | 1H NMR (300 |
| | | | | | | MHz, DMSO) d |
| | | | | | | 7.20 (s, 1H), 3.55- |
| | | | | | | 2.71 (m, 8H), |
| | | | | | | 3.45 (s, 3H), 2.75 |
| | | | | | | (s, 2H), 2.15- |
| | | | | | | 2.05 (m, 1H), |
| | | | | | | 1.63-1.50 (m, |
| | | | | | | 5H), 1.32 (s, 9H), |
| | | | | | | 1.33-1.22 (m, |
| | | | | | | 2H), 0.75 (d, 3H), |
| | | | | | | 0.70-0.60 (m, |
| | | | | | | 2H). |
| 120 | B | | | 431.24 | 1.15 | 1H NMR (300 |
| | | | | | | MHz, CDCl3) d |
| | | | | | | 7.04 (s, 1H), 6.59 |
| | | | | | | (d, J = 5.5 Hz, |
| | | | | | | 1H), 4.67 (dd, |
| | | | | | | J = 14.7, 9.8 Hz, |
| | | | | | | 1H), 3.97-3.78 |
| | | | | | | (m, 1H), 3.68 (t, |
| | | | | | | J = 8.7 Hz, 1H), |
| | | | | | | 2.90-2.71 (m, |
| | | | | | | 1H), 2.23-1.67 |
| | | | | | | (m, 7H), 1.61- |
| | | | | | | 1.38 (m, 2H), |
| | | | | | | 1.32 (s, 9H), |
| | | | | | | 1.03-0.82 (m, 5H). |

TABLE 2
IC₅₀, CC₅₀ LCMS and NMR data of the compounds described in FIG. 2

| | HCV- | | | | | |
|---|---|---|---|---|---|---|
| | Replicon | HCV- | | | | |
| | (Luci- | Replicon- | | | | |
| Compound | ET)- | ELISA-1a | [³H]Thymidine | LCMS | LCMS | |
| Nos. | 1b_IC50 | IC50 | CC50 | [M + H]⁺ | RT | NMR |
| 121 | C | | A | | 4.09 | (400 MHz, |
| | | | | | | DMSO-d6): |
| | | | | | | 13.42 (br s, |
| | | | | | | exchanged |
| | | | | | | with D2O; |
| | | | | | | 1H), 7.73- |
| | | | | | | 7.72 (m, 2H), |
| | | | | | | 7.57-7.41 (m, |
| | | | | | | 4H), 4.96 (d, |
| | | | | | | J = 16.4 Hz; |
| | | | | | | 1H), 3.93 (d, |
| | | | | | | J = 16.8 Hz; |
| | | | | | | 1H), 3.55- |
| | | | | | | 3.40 (m, 8H), |
| | | | | | | 2.22-2.16 (m, |
| | | | | | | 1H), 1.75-1.72 |
| | | | | | | (m, 1H), 1.57 |
| | | | | | | (br s, 2H), |
| | | | | | | 1.42-1.23 (m, |
| | | | | | | 4H), 0.75 (d, |
| | | | | | | J = 6 H |
| 122 | A | | A | 503.199 | 5.36 | (400 MHz, |
| | | | | | | DMSO-d6): |
| | | | | | | 13.65 (bs, |
| | | | | | | exchanged |
| | | | | | | with D2O; |
| | | | | | | 1H), 7.06 (bs, |
| | | | | | | 1H), 5.15 (bs, |
| | | | | | | 1H), 3.69- |
| | | | | | | 3.44 (m, 8H), |
| | | | | | | 2.08-2.04 (m, |
| | | | | | | 1H), 1.68- |
| | | | | | | 1.38 (m, 6H), |
| | | | | | | 1.35-0.87 (m, |
| | | | | | | 12H), 0.85- |
| | | | | | | 0.53 (m, 8H). |
| | | | | | | [1], (400 |
| | | | | | | MHz, DMSO- |
| | | | | | | d6): 13.63 (br |
| | | | | | | s, exchanged |
| | | | | | | with D2O; |
| | | | | | | 1H), 13.39 (br s, |
| | | | | | | exchanged |
| | | | | | | with D2O; |
| | | | | | | 1H), 7.26 (br |
| | | | | | | s, 1H), 7.14 |
| | | | | | | (br s, 1H), |
| | | | | | | 5.56 (m, 1H), |
| | | | | | | 5.20 (t, J = 6.8 |
| | | | | | | Hz; 1H), |
| | | | | | | 3.69-3.41 (m, |
| | | | | | | 8H), 3.1 (m, |
| | | | | | | 0.8H), 2.68 |
| | | | | | | (s, 1H), 1.99- |
| | | | | | | 1.96 (m, 2H), |
| | | | | | | 1.59-1.41 |
| | | | | | | (m, 6H),] |
| 123 | A | | A | 489 | 4.21 | (400 MHz, |
| | | | | | | DMSO-d6): |
| | | | | | | 13.47 (br s, |
| | | | | | | exchanged |
| | | | | | | with D2O; |
| | | | | | | 1H), 7.37 (d, |
| | | | | | | J = 8.4 Hz; |
| | | | | | | 2H), 7.26 (d, |
| | | | | | | J = 8.4 Hz; |
| | | | | | | 2H), 7.12 (s, |
| | | | | | | 1H), 5.12- |
| | | | | | | 5.08 (m, 1H), |
| | | | | | | 4.24-4.20 (m, |
| | | | | | | 1H), 3.59- |
| | | | | | | 3.46 (m, 8H), |
| | | | | | | 1.27 (s, 9H). |
| 124 | A | A | A | 523 | 2.73 | (400 MHz, |
| | | | | | | DMSO-d6): |
| | | | | | | 13.7 (br s, |
| | | | | | | exchanged |
| | | | | | | with D2O; |
| | | | | | | 1H), 7.62- |
| | | | | | | 7.61 (m, 1H), |
| | | | | | | 7.40-7.38 (m, |
| | | | | | | 1H), 7.23- |
| | | | | | | 7.20 (m, 1H), |
| | | | | | | 7.12 (s, 1H), |
| | | | | | | 5.15-5.11 (m, |
| | | | | | | 1H), 4.23- |
| | | | | | | 4.19 (m, 1H), |
| | | | | | | 3.58-3.44 |
| | | | | | | (m, 8H), 1.24 |
| | | | | | | (s, 9H). |
| 125 | B | | A | | 4.08 | (400 MHz, |
| | | | | | | DMSO-d6): |
| | | | | | | 13.4 (br s, |
| | | | | | | exchanged |
| | | | | | | with D2O; |
| | | | | | | 1H), 7.16- |
| | | | | | | 7.06 (m, 5H), |
| | | | | | | 5.11-5.06 m, |
| | | | | | | 1H), 4.21- |
| | | | | | | 4.17 (m, 1H), |
| | | | | | | 3.59-3.46 (m, |
| | | | | | | 8H), 2.25 (s, |
| | | | | | | 3H), 1.26 (s, |
| | | | | | | 9H). |
| 126 | B | | A | | 4.29 | (400 MHz, |
| | | | | | | DMSO-d6): |
| | | | | | | 13.4 (br s, |
| | | | | | | exchanged |
| | | | | | | with D2O; |
| | | | | | | 2H), 7.80- |
| | | | | | | 7.71 (m, 2H), |
| | | | | | | 7.50-7.43 (m, |
| | | | | | | 4H), 5.61 (br |
| | | | | | | s, 1H), 5.29- |
| | | | | | | 5.25 (m, 1H), |
| | | | | | | 3.72-3.40 (m, |
| | | | | | | 8H), 2.11- |
| | | | | | | 2.05 (m, |
| | | | | | | 1H), 1.85-1.82 |
| | | | | | | (m, 1H), |
| | | | | | | 1.71-1.40 (m, |
| | | | | | | 6H), 1.32- |
| | | | | | | 1.15 (m, 2H), |
| | | | | | | 0.91-0.81 (m, |
| | | | | | | 3H), 0.77-0.5 |
| | | | | | | [1] |
| 127 | A | | B | 491.199 | 4.85 | (400 MHz, |
| | | | | | | DMSO-d6): |
| | | | | | | 13.45 (br s, |
| | | | | | | exchanged |
| | | | | | | with D2O; |
| | | | | | | 1H), 7.21 (s, |
| | | | | | | 1H), 4.90 (d, |
| | | | | | | J = 16.8 Hz; |
| | | | | | | 1H), 3.84 (d, |
| | | | | | | J = 14.4 Hz; |
| | | | | | | 1H), 3.72- |
| | | | | | | 3.63 (m, 4H), |
| | | | | | | 2.64-2.59 (m, |
| | | | | | | 4H), 2.12- |
| | | | | | | 2.06 (m, 2H), |
| | | | | | | 1.69-1.53 (m, |
| | | | | | | 4H), 1.40- |
| | | | | | | 1.33 (m, |
| | | | | | | 11H), 0.77 (d, |
| | | | | | | J = 6.4 Hz; |
| | | | | | | 3H), 0.70- |
| | | | | | | 0.61 (m, 2 [1] |
| 128 | A | | A | | 1.34 | (400 MHz, |
| | | | | | | DMSO-d6): |
| | | | | | | 13.45 (br s, |
| | | | | | | exchanged |
| | | | | | | with D2O; |
| | | | | | | 1H), 4.91 (d, |
| | | | | | | J = 16.4 Hz; |
| | | | | | | 1H), 3.84 (d, |
| | | | | | | J = 16.4 Hz; |
| | | | | | | 1H), 3.54- |
| | | | | | | 3.53 (m, 4H), |
| | | | | | | 3.39-3.31 (m, |
| | | | | | | 4H), 2.10- |
| | | | | | | 2.05 (m, |
| | | | | | | 1H), 1.68-1.48 |
| | | | | | | (m, 5H), |
| | | | | | | 1.40-1.23 (m, |
| | | | | | | 4H), 0.97- |
| | | | | | | 0.92 (m, 2H), |
| | | | | | | 0.88-0.79 (m, |
| | | | | | | 2H), 0.77 (d, |
| | | | | | | J = 6.4 [1] |
| 129 | B | | A | 491.47 | 1.3 | (400 MHz, |
| | | | | | | DMSO-d6): |
| | | | | | | 13.62 (br s, |
| | | | | | | exchanged |
| | | | | | | with D2O; |
| | | | | | | 1H), 7.33 (s, |
| | | | | | | 1H), 4.92 (d, |
| | | | | | | J = 16.4 Hz; |
| | | | | | | 1H), 3.87 (d, |
| | | | | | | J = 16.8 Hz; |
| | | | | | | 1H), 3.54- |
| | | | | | | 3.40 (m, 8H), |
| | | | | | | 3.29 (s, 3H), |
| | | | | | | 2.09 (m, |
| | | | | | | 1H), 1.65-1.52 |
| | | | | | | (m, 4H), 1.48 |
| | | | | | | (s, 6H), 1.39- |
| | | | | | | 1.23 (m, 4H), |
| | | | | | | 0.77 (d, |
| | | | | | | J = 6.4 Hz; |
| | | | | | | 3H), 0.68- |
| | | | | | | 0.65 [1] |
| 130 | B | B | A | | 5.44 | (400 MHz, |
| | | | | | | DMSO-d6): |
| | | | | | | 13.5 (br s, |
| | | | | | | exchanged |
| | | | | | | with D2O; |
| | | | | | | 1H), 7.22 (s, |
| | | | | | | 1H), 4.91 (d, |
| | | | | | | J = 16.8 Hz; |
| | | | | | | 1H), 3.86 (d, |
| | | | | | | J = 16.8 Hz; |
| | | | | | | 1H), 3.34- |
| | | | | | | 3.40 (m, 8H), |
| | | | | | | 2.41 (d, |
| | | | | | | J = 6.4 Hz; |
| | | | | | | 2H), 2.11- |
| | | | | | | 2.06 (m, 1H), |
| | | | | | | 1.90-1.83 (m, |
| | | | | | | 1H), 1.65- |
| | | | | | | 1.52 (m, 3H), |
| | | | | | | 1.38-1.23 (m, |
| | | | | | | 3H), 0.99 (s, |
| | | | | | | 3H), [1] |
| 131 | A | A | A | | 1.41 | 400 MHz, |
| | | | | | | DMSO-d6: |
| | | | | | | 13.60 (br s, |
| | | | | | | exchanged |
| | | | | | | with D2O; |
| | | | | | | 1H), 7.12 (s, |
| | | | | | | 0.33H), 6.97 |
| | | | | | | (s, 0.65H), |
| | | | | | | 5.50 (t, |
| | | | | | | J = 9.6 Hz; |
| | | | | | | 0.64H), 4.10- |
| | | | | | | 4.02 (m, |
| | | | | | | 0.47H), 3.32- |
| | | | | | | 3.11 (m, 3H), |
| | | | | | | 3.04-3.00 (s, |
| | | | | | | 1H), 2.41- |
| | | | | | | 1.90 (m, 3H), |
| | | | | | | 1.60-1.45 (m, |
| | | | | | | 5H), 1.35- |
| | | | | | | 1.01 (m, |
| | | | | | | 11H), 1.01- |
| | | | | | | 0.86 (m, 3H), |
| | | | | | | 0. [1] |
| 132 | B | | A | | 1.24 | 400 MHz, |
| | | | | | | DMSO-d6: |
| | | | | | | 13.5 (s, |
| | | | | | | exchanged |
| | | | | | | with D2O; |
| | | | | | | 1H), 7.24 (s, |
| | | | | | | 1H), 4.92 (d, |
| | | | | | | J = 16.4 Hz; |
| | | | | | | 1H), 3.85 (d, |
| | | | | | | J = 16.4 Hz; |
| | | | | | | 1H), 3.54- |
| | | | | | | 3.53 (m, 4H), |
| | | | | | | 3.39-3.31 (m, |
| | | | | | | 4H), 2.52- |
| | | | | | | 2.46 (m, 2H), |
| | | | | | | 2.08-2.05 (m, |
| | | | | | | 1H), 1.65- |
| | | | | | | 1.51 (m, 4H), |
| | | | | | | 1.38-1.23 (m, |
| | | | | | | 3H), 1.16 (t, |
| | | | | | | J = 3.6 Hz; |
| | | | | | | 3H), 0.7 [1] |
| 133 | A | A | A | 489.449 | 1.85 | 400 MHz, |
| | | | | | | DMSO-d6: |
| | | | | | | 13.5 (br s, |
| | | | | | | exchanged |
| | | | | | | with D2O; |
| | | | | | | 1H), 7.12 (s, |
| | | | | | | 0.3H), 6.97 (s, |
| | | | | | | 0.7H), 5.56- |
| | | | | | | 5.51 (m, |
| | | | | | | 0.7H), 4.14- |
| | | | | | | 4.09 (m, |
| | | | | | | 0.3H), 3.45- |
| | | | | | | 3.36 (m, 3H), |
| | | | | | | 3.28-3.27 (m, |
| | | | | | | 2H), 3.25- |
| | | | | | | 3.23 (m, 1H), |
| | | | | | | 3.20-3.16 (m, |
| | | | | | | 2H), 3.13- |
| | | | | | | 3.09 (m, 1H), |
| | | | | | | 2.32-2.18 (m, |
| | | | | | | 1H), 2.10- |
| | | | | | | 1.96 [1], |
| | | | | | | 400 MHz, |
| | | | | | | DMSO-d6: |
| | | | | | | 13.6 (br s, |
| | | | | | | exchanged |
| | | | | | | with D2O; |
| | | | | | | 0.76H), 7.12 |
| | | | | | | (s, 0.3H), |
| | | | | | | 6.98 (s, 0.7H), |
| | | | | | | 5.56-5.52 (m, |
| | | | | | | 0.7H), 4.14- |
| | | | | | | 4.09 (m, |
| | | | | | | 0.3H), 3.43- |
| | | | | | | 3.20 (m, 9H), |
| | | | | | | 3.14-3.09 (m, |
| | | | | | | 1H), 2.32- |
| | | | | | | 1.96 (m, 3H), |
| | | | | | | 1.60-1.42 (m, |
| | | | | | | 5H), 1.29- |
| | | | | | | 1.12 (m, 11H), |
| | | | | | | 0.77 (d, |
| | | | | | | J = 6.4 Hz; |
| | | | | | | 3H), 0.71 |
| 134 | A | A | B | 487.74 | 3.94 | 1H NMR (300 |
| | | | | | | MHz, CDCl3) |
| | | | | | | 6.74 (s, 1H), |
| | | | | | | 5.07 (dd, J = |
| | | | | | | 12.0, 1.9 Hz, |
| | | | | | | 1H), 3.81 (dd, |
| | | | | | | J = 15.2, 10.7 |
| | | | | | | Hz, 1H), 3.71- |
| | | | | | | 3.20 (m, |
| | | | | | | 6H), 2.19- |
| | | | | | | 1.37 (m, 7H), |
| | | | | | | 1.34 (d, J = |
| | | | | | | 3.9 Hz, 9H), |
| | | | | | | 1.17 (q, J = |
| | | | | | | 7.2 Hz, 4H), |
| | | | | | | 0.85-0.62 |
| | | | | | | (m, 5H). |
| 135 | A | | A | 535.54 | 3.88 | |
| 136 | A | A | A | 475.65 | 3.7 | |
| 137 | A | A | A | 521.52 | 3.76 | 1H NMR (300 |
| | | | | | | MHz, CDCl3) |
| | | | | | | d 7.43 (d, J = |
| | | | | | | 8.3 Hz, 1H), |
| | | | | | | 7.35 (d, J = |
| | | | | | | 7.8 Hz, 1H), |
| | | | | | | 7.28-7.25 |
| | | | | | | (m, 1H), 7.22 |
| | | | | | | (s, 1H), 7.13 |
| | | | | | | (d, J = 1.7 |
| | | | | | | Hz, 1H), 6.97 |
| | | | | | | (s, 1H), 5.37 |
| | | | | | | (d, J = 11.3 |
| | | | | | | Hz, 1H), 3.98- |
| | | | | | | 3.68 (m, |
| | | | | | | 2H), 3.33 (t, J = |
| | | | | | | 12.6 Hz, |
| | | | | | | 1H), 3.13 (d, |
| | | | | | | J = 6.9 Hz, |
| | | | | | | 3H), 1.86 (s, |
| | | | | | | 6H), 1.33 (dd, |
| | | | | | | J = 7.1, 1.7 |
| | | | | | | Hz, 9H). [1] |
| 138 | A | A | A | 517.68 | 3.9 | 1H NMR (300 |
| | | | | | | MHz, CDCl3) |
| | | | | | | d 6.74 (s, |
| | | | | | | 1H), 5.09 (dd, |
| | | | | | | J = 12.0, 2.2 |
| | | | | | | Hz, 1H), 3.93- |
| | | | | | | 3.41 (m, |
| | | | | | | 6H), 3.35 (d, |
| | | | | | | J = 1.9 Hz, |
| | | | | | | 3H), 2.22- |
| | | | | | | 1.41 (m, |
| | | | | | | 14H), 1.35 (d, |
| | | | | | | J = 3.5 |
| | | | | | | Hz, 9H), 0.82 |
| | | | | | | (dd, J = 6.5, |
| | | | | | | 3.0 Hz, 3H), |
| | | | | | | 0.74 (s, 1H). |
| | | | | | | [1] |
| 139 | A | A | A | 475.55 | 1.7 | (DMSO-d6. |
| | | | | | | 400 MHz): |
| | | | | | | 13.6 (br s, |
| | | | | | | exchanged |
| | | | | | | with D2O, |
| | | | | | | 1H), 7.10 (s, |
| | | | | | | 0.31H), 6.97 |
| | | | | | | (s, 0.64H), |
| | | | | | | 5.55 (t, |
| | | | | | | J = 10 Hz; |
| | | | | | | 0.65H), 4.68- |
| | | | | | | 4.61 (m, |
| | | | | | | exchanged |
| | | | | | | with D2O, |
| | | | | | | 1H), 4.05 (t, |
| | | | | | | J = 9.6 Hz; |
| | | | | | | 0.30 H), 3.45- |
| | | | | | | 3.35 (m, 4H), |
| | | | | | | 3.13-3.03 (m, |
| | | | | | | 4H), 2.19- |
| | | | | | | 1.95 (m, 3H), |
| | | | | | | 1.57-1.33 (m, |
| | | | | | | 6H), [1] |
| 140 | A | A | A | 507.04 | 4.28 | performed in |
| | | | | | | DMSO-d6 |
| | | | | | | variable temp |
| | | | | | | experiment |
| | | | | | | attached |
| | | | | | | 1H NMR (300 |
| | | | | | | MHz, DMSO) |
| | | | | | | d 7.46 (d, J = |
| | | | | | | 8.2 Hz, 1H), |
| | | | | | | 7.39 (s, 1H), |
| | | | | | | 7.27 (d, 1H), |
| | | | | | | 7.15 (s, 1H), |
| | | | | | | 4.65 (s, 1H), |
| | | | | | | 3.28 (s, 2H), |
| | | | | | | 2.90 (s, 3H), |
| | | | | | | 2.35 (s, 1H), |
| | | | | | | 2.20-2.03 |
| | | | | | | (m, 1H), 1.88 |
| | | | | | | (s, 2H), 1.30 |
| | | | | | | (d, J = 3.5 |
| | | | | | | Hz, 9H). [1] |
| 141 | A | | A | 473.19 | 1.39 | 400 MHz, |
| | | | | | | DMSO-d6: |
| | | | | | | 13.6 (br s, |
| | | | | | | exchanged |
| | | | | | | with D2O; |
| | | | | | | 1H), 7.13 (s, |
| | | | | | | 0.48H, 6.96 |
| | | | | | | (s, 1H), 5.48 |
| | | | | | | (t, J = 9 Hz; |
| | | | | | | 1H), 4.11- |
| | | | | | | 4.07 (m, 3H), |
| | | | | | | 3.34-3.31 (m, |
| | | | | | | 0.57H), 3.16- |
| | | | | | | 3.10 (m, |
| | | | | | | 1.62H), 2.98- |
| | | | | | | 2.93 (m, 1H), |
| | | | | | | 2.32-1.96 (m, |
| | | | | | | 4H), 1.60- |
| | | | | | | 1.43 (m, 8H), |
| | | | | | | 1.29 (s, 9H), |
| | | | | | | 1.05-1.01 (m, |
| | | | | | | 6 [1] |
| 142 | A | A | B | 473.19 | 1.39 | 400 MHz, |
| | | | | | | DMSO-d6: |
| | | | | | | 13.6 (br s, |
| | | | | | | exchanged |
| | | | | | | with D2O; |
| | | | | | | 1H), 7.13 (s, |
| | | | | | | 0.48H, 6.96 |
| | | | | | | (s, 1H), 5.48 |
| | | | | | | (t, J = 9 Hz; |
| | | | | | | 1H), 4.11- |
| | | | | | | 4.07 (m, 3.67 |
| | | | | | | H), 3.36-3.32 |
| | | | | | | (m, 0.83H), |
| | | | | | | 3.16-3.10 (m, |
| | | | | | | 1.8H), 2.97- |
| | | | | | | 2.93 (m, 1H), |
| | | | | | | 2.32-1.96 (m, |
| | | | | | | 4H), 1.60- |
| | | | | | | 1.43 (m, 8H), |
| | | | | | | 1.29 (s, 9H), |
| | | | | | | 1.05-1.01 (m |
| | | | | | | [1] |
| 143 | A | A | B | 551.07 | 3.82 | 1H NMR (400 |
| | | | | | | MHz, CDCl3) |
| | | | | | | d 7.68 (s, |
| | | | | | | 0.5H), 7.29 |
| | | | | | | (m, 2H), 7.22- |
| | | | | | | 7.04 (m, |
| | | | | | | 1H), 6.96 (s, |
| | | | | | | 0.5H), 5.49 |
| | | | | | | (m, 1H), 4.26- |
| | | | | | | 3.42 (m, |
| | | | | | | 8H), 1.96- |
| | | | | | | 1.45 (m, 2H), |
| | | | | | | 1.31 (s, 9H), |
| | | | | | | 0.96 (m, 3H). |
| | | | | | | [1] |
| 144 | A | | A | | 2.06 | 400 MHz, |
| | | | | | | DMSO: 13.45 |
| | | | | | | (s, |
| | | | | | | exchanged |
| | | | | | | with D2O; |
| | | | | | | 1H), 7.21 (s, |
| | | | | | | 1H), 4.91 (d, |
| | | | | | | J = 16.4 Hz; |
| | | | | | | 1H), 3.84 (d, |
| | | | | | | J = 16.4 Hz; |
| | | | | | | 1H), 3.54- |
| | | | | | | 3.53 (m, 4H), |
| | | | | | | 3.39-3.32 (m, |
| | | | | | | 4H), 2.11- |
| | | | | | | 1.98 (m, 1H), |
| | | | | | | 1.60-1.51 (m, |
| | | | | | | 4H), 1.37- |
| | | | | | | 1.34 (m, 2H), |
| | | | | | | 1.31 (s, 3H), |
| | | | | | | 1.19-1.17 (m, |
| | | | | | | 2H), 1.02- |
| | | | | | | 1.00 (m, 2 [1] |
| 145 | A | | B | 530.42 | 2.28 | 1H NMR (300 |
| | | | | | | MHz, MeOD) |
| | | | | | | d 7.38 (s, |
| | | | | | | 1H), 5.21 (d, |
| | | | | | | J = 11.6 Hz, |
| | | | | | | 1H), 4.41- |
| | | | | | | 4.15 (m, 1H), |
| | | | | | | 3.86-3.63 |
| | | | | | | (m, 1H), 3.59- |
| | | | | | | 3.35 (m, |
| | | | | | | 3H), 3.00 (d, |
| | | | | | | J = 14.3 Hz, |
| | | | | | | 6H), 2.22- |
| | | | | | | 2.20 (m, 1H), |
| | | | | | | 1.74 (dt, J = |
| | | | | | | 28.5, 12.7 |
| | | | | | | Hz, 6H), 1.33 |
| | | | | | | (s, 9H), 1.17 |
| | | | | | | (s, 2H), 0.82 |
| | | | | | | (d, J = 6.5 |
| | | | | | | Hz, 3H), 0.77- |
| | | | | | | 0.48 (m, |
| | | | | | | 2H). |
| 146 | A | A | B | 501.43 | 2.12 | 1H NMR (300 |
| | | | | | | MHz, CDCl3) |
| | | | | | | d 6.65 (s, |
| | | | | | | 1H), 5.00- |
| | | | | | | 4.92 (m, 1H), |
| | | | | | | 4.84 (s, 2H), |
| | | | | | | 3.51-3.27 |
| | | | | | | (m, 2H), 2.28- |
| | | | | | | 2.11 (m, |
| | | | | | | 1H), 2.07- |
| | | | | | | 1.87 (m, 3H), |
| | | | | | | 1.86-169 |
| | | | | | | (m, 4H), 1.67- |
| | | | | | | 1.44 (m, |
| | | | | | | 4H), 1.49- |
| | | | | | | 1.31 (m, 3H), |
| | | | | | | 1.26 (d, J = |
| | | | | | | 3.0 Hz, 9H), |
| | | | | | | 1.06 (dt, J = |
| | | | | | | 13.7, 6.8 Hz, |
| | | | | | | 6H), 0.81 (s, |
| | | | | | | 1H), 0.75- |
| | | | | | | 0.69 (m, 3H), |
| | | | | | | 0.66 (s, 2H). |
| 147 | B | | A | 523.19 | 3.51 | 1H NMR (300 |
| | | | | | | MHz, CDCl3) |
| | | | | | | d 7.64-7.54 |
| | | | | | | (m, 1H), 7.58 |
| | | | | | | (d, J = 7.0 |
| | | | | | | Hz, 1H), 7.33 |
| | | | | | | (s, 1H), 7.27- |
| | | | | | | 7.20 (m, 1H), |
| | | | | | | 7.17 (dd, J = |
| | | | | | | 8.3, 1.9 Hz, |
| | | | | | | .5H), 7.04 (s, |
| | | | | | | .5H), 4.63 (d, |
| | | | | | | J = 11.7 Hz, |
| | | | | | | 1H), 4.38 (s, |
| | | | | | | 2H), 4.06 (s, |
| | | | | | | 1H), 3.90 (dd, |
| | | | | | | J = 15.2, 10.4 |
| | | | | | | Hz, 1H), 3.69 |
| | | | | | | (s, 1H), 3.31 |
| | | | | | | (ddd, J = |
| | | | | | | 33.6, 15.7, |
| | | | | | | 3.4 Hz, 1H), |
| | | | | | | 3.18-3.08 |
| | | | | | | (m, 3H), 1.34- |
| | | | | | | 1.24 (m, |
| | | | | | | 9H). |
| 148 | A | A | A | 473.15 | 1.61 | 1H NMR (400 |
| | | | | | | MHz, CDCl3) |
| | | | | | | d 6.76 (s, |
| | | | | | | 1H), 3.68- |
| | | | | | | 3.58 (m, 1H), |
| | | | | | | 3.51 (m, 1H), |
| | | | | | | 3.41 (m, 2H), |
| | | | | | | 3.18 (m, 1H), |
| | | | | | | 2.37 (dd, J = |
| | | | | | | 23.5, 12.2 |
| | | | | | | Hz, 1H), 2.08 |
| | | | | | | (m, 2H), 1.95 |
| | | | | | | (d, J = 13.6 |
| | | | | | | Hz, 1H), 1.73 |
| | | | | | | (m, 2H), 1.62- |
| | | | | | | 1.41 (m, |
| | | | | | | 4H), 1.26 (s, |
| | | | | | | 9H), 1.12 (m, |
| | | | | | | 5H), 0.73 (d, |
| | | | | | | J = 6.5 Hz, |
| | | | | | | 3H), 0.69- |
| | | | | | | 0.57 (m, 2H). |
| 149 | A | A | B | 487.17 | 1 | 1H NMR (400 |
| | | | | | | MHz, CDCl3) |
| | | | | | | d 6.63 (s, |
| | | | | | | 1H), 4.78- |
| | | | | | | 4.64 (m, 1H), |
| | | | | | | 3.66-3.47 |
| | | | | | | (m, 1H), 3.21- |
| | | | | | | 3.05 (m, |
| | | | | | | 2H), 2.16 (m, |
| | | | | | | 1H), 2.02- |
| | | | | | | 1.80 (m, 4H), |
| | | | | | | 1.61 (m, 1H), |
| | | | | | | 1.36 (m, 3H), |
| | | | | | | 1.16-1.08 |
| | | | | | | (s, 9H), 1.09- |
| | | | | | | 0.99 (m, 6H), |
| | | | | | | 0.96-0.90 |
| | | | | | | (m, 3H), 0.62 |
| | | | | | | (d, J = 6.5 |
| | | | | | | Hz, 3H), 0.54 |
| | | | | | | (m, 2H). |
| 150 | A | | A | 503.14 | 3.71 | 1H NMR (400 |
| | | | | | | MHz, CDCl3) |
| | | | | | | d 6.58 (m, |
| | | | | | | 1H), 3.53- |
| | | | | | | 3.39 (m, 2H), |
| | | | | | | 3.35 (m, 2H), |
| | | | | | | 3.26 (m, 3H), |
| | | | | | | 3.12 (s, 3H), |
| | | | | | | 2.23 (m, 1H), |
| | | | | | | 1.91 (m, 2H), |
| | | | | | | 1.76 (m, 1H), |
| | | | | | | 1.55 (m, 1H), |
| | | | | | | 1.46-1.24 |
| | | | | | | (m, 4H), 1.10 |
| | | | | | | (s, 6H), 0.98 |
| | | | | | | (s, 5H), 0.57 |
| | | | | | | (d, J = 6.4 |
| | | | | | | Hz, 3H), 0.51 |
| | | | | | | (m, 3H). |
| 151 | B | | B | 563.039 | 2.43 | (400 MHz, |
| | | | | | | DMSO-d6): |
| | | | | | | 13.3 (br s, |
| | | | | | | exchanged |
| | | | | | | with D2O; |
| | | | | | | 1H), 7.75- |
| | | | | | | 7.73 (m, 2H), |
| | | | | | | 7.50 (s, 1H), |
| | | | | | | 7.45-7.41 (m, |
| | | | | | | 2H), 7.22- |
| | | | | | | 7.18 (m, 1H), |
| | | | | | | 7.09-7.07 |
| | | | | | | (4H), 4.95 (d, |
| | | | | | | J = 16.4 Hz; |
| | | | | | | 1H), 3.92 (d, |
| | | | | | | J = 15.6 Hz; |
| | | | | | | 1H), 3.55- |
| | | | | | | 3.36 (m, 8H), |
| | | | | | | 2.22-2.16 (m, |
| | | | | | | 1H), 1.75- |
| | | | | | | 1.72 (m, 1H), |
| | | | | | | 1.60 |
| 152 | C | | B | 610.85 | 2.37 | (400 MHz, |
| | | | | | | DMSO-d6): |
| | | | | | | 13.5 (br s, |
| | | | | | | exchanged |
| | | | | | | with D2O; |
| | | | | | | 1H), 7.57- |
| | | | | | | 7.53 (m, 3H), |
| | | | | | | 7.44-7.36 (m, |
| | | | | | | 4H), 7.31- |
| | | | | | | 7.29 (m, 1H), |
| | | | | | | 7.21-7.17 (m, |
| | | | | | | 1H), 7.08- |
| | | | | | | 7.03 (m, 4H), |
| | | | | | | 5.19-5.15 (m, |
| | | | | | | 1H), 4.31- |
| | | | | | | 4.27 (m, 1H), |
| | | | | | | 3.60-3.45 (m, |
| | | | | | | 8H). |
| 153 | A | | A | 473.34 | 3.87 | 1H NMR (300 |
| | | | | | | MHz, CDCl3) |
| | | | | | | ? 7.61 (s, |
| | | | | | | 0.6H), 6.75 |
| | | | | | | (s, 0.4H), |
| | | | | | | 5.30 (brs, |
| | | | | | | 0.6H), 5.08 |
| | | | | | | (d, J = 10.6 |
| | | | | | | Hz, 0.4H), |
| | | | | | | 4.00-3.61 |
| | | | | | | (m, 1H), 3.39- |
| | | | | | | 3.13 (m, |
| | | | | | | 1H), 3.09 (s, |
| | | | | | | 1.2H), 3.05 |
| | | | | | | (s, 1.8H), |
| | | | | | | 2.23-1.39 |
| | | | | | | (m, 14H), |
| | | | | | | 1.34 (two |
| | | | | | | singlets, 9H), |
| | | | | | | 0.75 (m, 5H). |
| | | | | | | 1H NMR (300 |
| | | | | | | MHz, MeOD) |
| | | | | | | ? 8.03-7.92 |
| | | | | | | (m, 1H), 7.45 |
| | | | | | | (s, 1H), 5.28 |
| | | | | | | (d, 1H), 3.89- |
| | | | | | | 3.67 (m, |
| | | | | | | 1H), 3.00 (d, |
| | | | | | | J = 5.4 Hz, |
| | | | | | | 3H), 2.20- |
| | | | | | | 2.02 (m, 1H), |
| | | | | | | 1.99-1.37 |
| | | | | | | (m, 10H), |
| | | | | | | 1.34 (s, 9H), |
| | | | | | | 1.18-0.98 |
| | | | | | | (m, 1H), 0.82 |
| | | | | | | (d, 3H), 0.77- |
| | | | | | | 0.56 (m, |
| | | | | | | 2H). |
| 154 | A | | | 459.24 | 1.46 | 1H NMR (300 |
| | | | | | | MHz, CDCl3) |
| | | | | | | ? 6.84 (s, |
| | | | | | | 1H), 3.80- |
| | | | | | | 3.48 (m, 2H), |
| | | | | | | 3.36-3.15 |
| | | | | | | (m, 1H), 3.05 |
| | | | | | | (s, 3H), 2.65- |
| | | | | | | 2.35 (m, 1H), |
| | | | | | | 2.35-1.93 |
| | | | | | | (m, 4H), 1.93- |
| | | | | | | 1.75 (m, |
| | | | | | | 2H), 1.74- |
| | | | | | | 1.45 (m, 4H), |
| | | | | | | 1.35 (s, 9H), |
| | | | | | | 0.84 (d, 3H), |
| | | | | | | 0.77-0.63 |
| | | | | | | (m, 1H). |

TABLE 3
IC₅₀, CC₅₀ LCMS and NMR data of the compounds described in FIG. 3

| | HCV- | | | | | |
|---|---|---|---|---|---|---|
| | Replicon | HCV- | | | | |
| | (Luci- | Replicon- | | | | |
| Compound | ET)- | ELISA-1a | [³H]Thymidine | LCMS | LCMS | |
| Nos. | 1b_IC50 | IC50 | CC50 | [M + H]⁺ | RT | NMR |
| 156 | A | 0.004 | | 501.259 | 1.87 | 400MHZ, DMSO-d6: |
| | | | | | | 6.85 (s, 0.27H), 6.69 (s, |
| | | | | | | 0.77H), 5.37-5.33 (m, |
| | | | | | | 0.3H), 4.58-4.53(m, 0.7H), |
| | | | | | | 3.81-3.71 (m, 2H), 3.65- |
| | | | | | | 3.54 (m, 2H), 3.32-3.13 (m, |
| | | | | | | 1H), 2.83-2.82(m, 1H), 2.14- |
| | | | | | | 1.98 (m, 3H), 1.95- |
| | | | | | | 1.77 (m, 3H), 1.57- |
| | | | | | | 1.50 (m, 3H), 1.50- |
| | | | | | | 1.40 (m, 1H), 1.27 (s, 9H), |
| | | | | | | 1.23-1.10 |
| 157 | | | | 586.6 | 2.35 | H NMR (300.0 MHz, |
| | | | | | | DMSO) d 7.52 (s, 1H), 5.23 |
| | | | | | | (d, J = 11.5 Hz, 1H), 4.39- |
| | | | | | | 4.33 (m, 1H), 4.29-4.23 |
| | | | | | | (m, 2H), 3.61 (d, J = 3.9 Hz, |
| | | | | | | 1H), 3.54 (t, J = 4.5 Hz, |
| | | | | | | 4H), 3.25 (dd, J = 4.3, 15.0 |
| | | | | | | Hz, 2H), 2.90 (s, 3H), 2.57 |
| | | | | | | (t, J = 5.2 Hz, 2H), 2.44- |
| | | | | | | 2.38 (m, 4H), 2.02-1.90 |
| | | | | | | (m, 1H), 1.85-1.66 (m, H), |
| | | | | | | 1.64 - 1.46 (m, 5H), 1.43- |
| | | | | | | 1.36 (m, 2H), 1.30 (s, 9H), |
| | | | | | | 1.23-0.83 (m, 4H), 0.76 (d, |
| | | | | | | J = 6.5 Hz, 3H) and 0.70- |
| | | | | | | 0.48 (m, 2H) ppm |
| 158 | | | | 587.62 | 3.45 | H NMR (300.0 MHz, |
| | | | | | | DMSO) d 7.55 (s, 1H), 5.91 |
| | | | | | | (d, J = 5.9 Hz, 1H), 5.79 (d, |
| | | | | | | J = 5.9 Hz, 1H), 5.21 (d, J = |
| | | | | | | 11.5 Hz, 1H), 3.59 (dd, J = |
| | | | | | | 11.2, 15.1 Hz, 1H), 3.24 |
| | | | | | | (dd, 4.0, 15.0 Hz, 1H), 2.90 |
| | | | | | | (s, 3H), 2.01-1.90 (m, 1H), |
| | | | | | | 1.81-1.65 (m, 2H), 1.63- |
| | | | | | | 1.48 (m, 5H), 1.45-1.31 |
| | | | | | | (m, 1H), 1.30 (s, 9H), 1.25- |
| | | | | | | 1.15 (m, 2H), 1.14 (s, 9H), |
| | | | | | | 1.08-0.84 (m, 3H), 0.75 (d, |
| | | | | | | J = 6.4 Hz, 3H) and 0.70- |
| | | | | | | 0.45 (m, 2H) ppm |
| 159 | | | | 589.6 | 3.06 | H NMR (400.0 MHz, |
| | | | | | | DMSO) d 7.55 (s, 1H), 5.91 |
| | | | | | | (d, J = 6.2 Hz, 1H), 5.78 (d, |
| | | | | | | J = 6.2 Hz, 1H), 5.23 (d, J = |
| | | | | | | 11.8 Hz, 1H), 4.81 (qn, J = |
| | | | | | | 6.2 Hz, 1H), 3.61 (dd, J = |
| | | | | | | 11.4, 15.1 Hz, 1H), 3.29- |
| | | | | | | 3.22 (m, 1H), 2.90 (s, 3H), |
| | | | | | | 1.94 (t, J = 11.6 Hz, 1H), |
| | | | | | | 1.79-1.33 (m, 10H), 1.30 |
| | | | | | | (s, 9H), 1.24 (dd, J = 1.5, |
| | | | | | | 6.2 Hz, 6H), 1.20-0.90 (m, |
| | | | | | | 3H), 0.76 (d, J = 6.4 Hz, |
| | | | | | | 3H) and 0.73-0.48 (m, 2H) |
| | | | | | | ppm |
| 160 | | | | 573.61 | 3.18 | H NMR (300.0 MHz, |
| | | | | | | DMSO) d 7.55 (s, 1H), 5.91 |
| | | | | | | (d, J = 6.0 Hz, 1H), 5.78 (d, |
| | | | | | | J = 6.0 Hz, 1H), 5.21 (d, J = |
| | | | | | | 11.5 Hz, 2H), 3.57 (d, J = |
| | | | | | | 11.1 Hz, 1H), 3.27 (d, J = |
| | | | | | | 2.9 Hz, 1H), 2.90 (s, 3H), |
| | | | | | | 2.60-2.50 (m, 1H), 2.01- |
| | | | | | | 1.87 (m, 1H), 1.82-1.33 |
| | | | | | | (m, 9H), 1.30 (s, 9H), 1.29- |
| | | | | | | 1.11 (m, 2H), 1.09 (dd, J = |
| | | | | | | 1.2, 7.0 Hz, 6H), 1.03-0.86 |
| | | | | | | (m, 1H), 0.76 (d, J = 6.4 Hz, |
| | | | | | | 3H) and 0.71-0.47 (m, 2H) |
| | | | | | | ppm |
| 161 | | | | 585.57 | 2.61 | H NMR (300.0 MHz, |
| | | | | | | DMSO) d 7.52 (s, 1H), 5.28- |
| | | | | | | 5.08 (m, 3H), 3.61 (dd, J = |
| | | | | | | 11.3, 15.1, 1H), 3.29-3.22 |
| | | | | | | (m, 1H), 2.90 (s, 3H), 2.17 |
| | | | | | | (s, 3H), 2.00-1.31 (m, 9H), |
| | | | | | | 1.30 (s, 9H), 1.28-0.85 (m, |
| | | | | | | 5H), 0.75 (d, J = 6.5 Hz, |
| | | | | | | 3H) and 0.70-0.46 (m, 2H) |
| | | | | | | ppm |
| 162 | | | | 589.6 | 3.04 | H NMR (300.0 MHz, |
| | | | | | | DMSO) d 7.27 (s, 1H), 5.89 |
| | | | | | | (d, J = 5.9 Hz, 1H), 5.82 (d, |
| | | | | | | J = 5.9 Hz, 1H), 4.92 (d, J = |
| | | | | | | 16.7 Hz, 1H), 3.85 (d, J = |
| | | | | | | 16.6 Hz, 1H), 3.55 (br s, |
| | | | | | | 4H), 3.41-3.35 (m, 4H), |
| | | | | | | 2.11-1.98 (m, 1H), 1.70- |
| | | | | | | 1.34 (m, 6H), 1.30 (s, 9H), |
| | | | | | | 1.28-1.20 (m, 1H), 1.14 (s, |
| | | | | | | 9H), 0.77 (d, J = 6.5 Hz, |
| | | | | | | 3H) and 0.74-0.57 (m, 2H) |
| | | | | | | ppm |
| 163 | | | | 617.56 | 3.49 | (1H NMR shows rotamers) |
| | | | | | | 1H NMR (300.0 MHz, |
| | | | | | | DMSO) d 7.34 (s, 0.6H), |
| | | | | | | 7.27 (s, 0.4H), 5.91-5.75 |
| | | | | | | (m, 2H), 5.56 (t, J = 7.5 Hz, |
| | | | | | | 0.4H), 5.19 (dd, J = 6.4, 8.6 |
| | | | | | | Hz, 0.6H), 3.77-3.39 (m, |
| | | | | | | 7.6H), 3.21-3.10 (m, |
| | | | | | | 0.4H), 2.00-1.82 (m, 1H), |
| | | | | | | 1.65-1.35 (m, 6H), 1.34- |
| | | | | | | 1.16 (m, 12H), 1.13 (s, 9H) |
| | | | | | | and 0.80-0.68 (m, 8H) |
| | | | | | | ppm |
| 164 | | | | 575.59 | 3.53 | (1H NMR shows rotamers) |
| | | | | | | 1H NMR (300.0 MHz, |
| | | | | | | DMSO) d 7.31 (s, 0.6H), |
| | | | | | | 7.24 (s, 0.4H), 5.90 (d, J = |
| | | | | | | 6.0 Hz, 0.6H), 5.81 (d, J = |
| | | | | | | 5.0 Hz, 1.4H), 5.58 (t, J = |
| | | | | | | 7.5 Hz, 0.4H), 5.16 (dd, J = |
| | | | | | | 6.2, 8.8 Hz, 0.6H), 3.10 (s, |
| | | | | | | 1.8H), 3.05 (s, 1.2H), 2.85 |
| | | | | | | (s, 1.8H), 2.65 (s, 1.2H), |
| | | | | | | 1.99-1.84 (m, 1H), 1.66- |
| | | | | | | 1.33 (m, 6H), 1.33-1.15 |
| | | | | | | (m, 12H), 1.14 (s, 9H) and |
| | | | | | | 0.79-0.49 (m, 8H) ppm |
| 165 | | | | 591.59 | 2.59 | 1H NMR (300.0 MHz, |
| | | | | | | DMSO) d 7.27 (s, 1H), 5.84 |
| | | | | | | (dd, J = 6.2, 9.7 Hz, 2H), |
| | | | | | | 4.91 (d, J = 16.7 Hz, 1H), |
| | | | | | | 4.80 (septet, J = 6.2 Hz, |
| | | | | | | 1H), 3.89 (d, J = 16.7 Hz, |
| | | | | | | 1H), 3.55 (br s, 4H), 3.45- |
| | | | | | | 3.35 (m, 4H), 2.13-1.98 |
| | | | | | | (m, 1H), 1.79-1.33 (m, |
| | | | | | | 7H), 1.30 (s, 9H), 1.24 (d, J = |
| | | | | | | 6.2 Hz, 6H), 0.77 (d, J = |
| | | | | | | 6.4 Hz, 3H) and 0.75-0.57 |
| | | | | | | (m, 2H) ppm |
| 166 | | | | 619.61 | 3.02 | (1H NMR shows rotamers) |
| | | | | | | 1H NMR (300.0 MHz, |
| | | | | | | DMSO) d 7.36 (s, 0.6H), |
| | | | | | | 7.27 (s, 0.4H), 5.89 (d, J = |
| | | | | | | 6.3 Hz, 0.6H), 5.81-5.74 |
| | | | | | | (m, 1.4H), 5.56 (t, J = 7.4 |
| | | | | | | Hz, 0 4H), 5.19 (dd, J = 6.5, |
| | | | | | | 8.5 Hz, 0.6H), 4.88-4.73 |
| | | | | | | (m, 2H), 3.73-3.35 (m, |
| | | | | | | 7.6H), 3.23-3.12 (m, |
| | | | | | | 0.4H), 1.97-1.84 (m, 1H), |
| | | | | | | 1.66-1.34 (m, 6H), 1.32- |
| | | | | | | 1.07 (m, 17H) and 0.81- |
| | | | | | | 0.47 (m, 8H) ppm |
| 167 | | | | 577.57 | 3.05 | (1H NMR shows rotamers) |
| | | | | | | 1H NMR (300.0 MHz, |
| | | | | | | DMSO) d 7.33 (s, 0.55H), |
| | | | | | | 7.24 (s, 0.45H), 5.89 (d, J = |
| | | | | | | 6.3 Hz, 0.55H), 5.81 (dd, J = |
| | | | | | | 6.3, 8.0 Hz, 1H), 5.73 (d, |
| | | | | | | J = 6.3 Hz, 0.45H), 5.58 (t, |
| | | | | | | J = 7.5 Hz, 0.45H), 5.16 (d, |
| | | | | | | J = 2.4 Hz, 0.55H), 4.87- |
| | | | | | | 4.85 (m, 1H), 3.11 (s, |
| | | | | | | 1.65H), 3.02 (s, 1.35H), |
| | | | | | | 2.85 (s, 1.65H), 2.63 (s, |
| | | | | | | 1.35H), 1.99-1.86 (m, 1H), |
| | | | | | | 1.67-1.35 (m, 6H), 1.34- |
| | | | | | | 1.08 (m, 18H) and 0.82- |
| | | | | | | 0.51 (m, 8H) ppm |
| 168 | | | | 587.55 | 2.25 | 1H NMR (300.0 MHz, |
| | | | | | | DMSO) d 7.24 (s, 1H), 5.18 |
| | | | | | | (d, J = 14.1 Hz, 1H), 5.12 |
| | | | | | | (d, J = 14.1 Hz, 1H), 4.89 |
| | | | | | | (d, J = 16.7 Hz, 1H), 3.92 |
| | | | | | | (d, J = 16.7 Hz, H), 3.61- |
| | | | | | | 3.48 (m, 4H), 3.47-3.33 |
| | | | | | | (m, 4H), 2.17 (s, 3H), 2.13- |
| | | | | | | 2.00 (m, 1H), 1.73-1.33 |
| | | | | | | (m, 5H), 1.30 (s, 9H), 1.30- |
| | | | | | | 1.15 (m, 2H), 0.77 (d, J = |
| | | | | | | 6.5 Hz, 3H) and 0.73-0.55 |
| | | | | | | (m, 2H) ppm |
| 169 | | | | 615.58 | 2.61 | 1H NMR (300.0 MHz, |
| | | | | | | DMSO) d 7.31 (s, 0.6H), |
| | | | | | | 7.24 (s, 0.4H), 5.55 (t, J = |
| | | | | | | 7.5 Hz, 0.4H), 5.25-5.05 |
| | | | | | | (m, 2.6H), 3.78-3.40 (m, |
| | | | | | | 7.6H), 3.09-2.99 (m, |
| | | | | | | 0.4H), 2.17 (s, 3H), 1.98- |
| | | | | | | 1.85 (m, 1H), 1.65-1.33 |
| | | | | | | (m, 6H), 1.32-1.05 (m, |
| | | | | | | 12H) and 0.81-0.47 (m, |
| | | | | | | 8H) ppm |
| 170 | | | | 573.61 | 2.66 | 1H NMR (300.0 MHz, |
| | | | | | | DMSO) d 7.28 (s, 0.6H), |
| | | | | | | 7.21 (s, 0.4H), 5.55 (t, J = |
| | | | | | | 7.5 Hz, 0.4H), 5.24-5.12 |
| | | | | | | (m, 2.2H), 5.02 (d, J = 14.2 |
| | | | | | | Hz, 0.4H), 3.10 (s, 1.8H), |
| | | | | | | 3.05 (s, 1.2H), 2.85 (s, |
| | | | | | | 1.8H), 2.64 (s, 1.2H), 2.17 |
| | | | | | | (s, 3H), 1.96-1.85 (m, 1H), |
| | | | | | | 1.66-1.33 (m, 6H), 1.32- |
| | | | | | | 1.05 (m, 12H) and 0.79- |
| | | | | | | 0.47 (m, 8H) ppm |
| 171 | | | | 575.59 | 2.9 | 1H NMR (300.0 MHz, |
| | | | | | | DMSO) d 7.26 (s, 1H), 5.85 |
| | | | | | | (dd, J = 5.9, 16.9 Hz, 2H), |
| | | | | | | 4.91 (d, J = 16.7 Hz, 1H), |
| | | | | | | 3.86 (d, J = 16.7 Hz, 1H), |
| | | | | | | 3.62-3.48 (m, 4H), 3.44- |
| | | | | | | 3.33 (m, 4H), 2.65-2.55 |
| | | | | | | (m, 1H), 2.13-1.98 (m, |
| | | | | | | 1H), 1.78-1.35 (m, 5H), |
| | | | | | | 1.30 (s, 9H), 1.29-1.18 (m, |
| | | | | | | 2H), 1.08 (d, J = 7.0 Hz, |
| | | | | | | 6H), 0.77 (d, J = 6.5 Hz, |
| | | | | | | 3H) and 0.74-0.58 (m, 2H) |
| | | | | | | ppm |
| 172 | | | | 603.68 | 3.36 | 1H NMR (300.0 MHz, |
| | | | | | | DMSO) d 7.34 (s, 0.6H), |
| | | | | | | 7.27 (s, 0.4H), 5.90 (d, J = |
| | | | | | | 6.0 Hz, 0.6H), 5.85-5.76 |
| | | | | | | (m, 1.4H), 5.56 (t, J = 7.3 |
| | | | | | | Hz, 0 4H), 5.18 (dd, J = 6.4, |
| | | | | | | 8.6 Hz, 0.6H), 3.75-3.35 |
| | | | | | | (m, 7.6H), 3.20-3.09 (m, |
| | | | | | | 0.4H), 2.62-2.53 (m, 1H), |
| | | | | | | 1.98-1.82 (m, 1H), 1.65- |
| | | | | | | 1.34 (m, 6H), 1.32-1.13 |
| | | | | | | (m, 12H), 1.10-1.06 (m, |
| | | | | | | 6H) and 0.82-0.49 (m, 8H) |
| | | | | | | ppm |
| 173 | | | | 561.64 | 3.41 | 1H NMR (300.0 MHz, |
| | | | | | | DMSO) d 7.31 (s, 0.6H), |
| | | | | | | 7.23 (s, 0.4H), 5.90 (d, J = |
| | | | | | | 6.0 Hz, 0.6H), 5.82-5.75 |
| | | | | | | (m, 1.4H), 5.58 (t, J = 7.5 |
| | | | | | | Hz, 0.4H), 5.15 (dd, J = 6.2, |
| | | | | | | 8.8 Hz, 0.6H), 3.10 (s, |
| | | | | | | 1.8H), 3.04 (s, 1.2H), 2.85 |
| | | | | | | (s, 1.8H), 2.65 (s, 1.2H), |
| | | | | | | 2.63-2.53 (m, 1H), 1.99- |
| | | | | | | 1.85 (m, 1H), 1.65-1.32 |
| | | | | | | (m, 6H), 1.32-1.12 (m, |
| | | | | | | 12H), 1.08 (d, J = 7.0 Hz, |
| | | | | | | 6H) and 0.79-0.47 (m, 8H) |
| | | | | | | ppm |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound represented by Structural Formula (I): 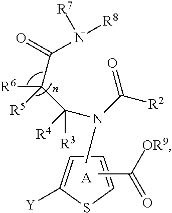
or a pharmaceutically acceptable salt thereof, wherein:
Ring A is optionally further substituted with one or more substituents selected from the group consisting of -D, halogen, —CN, C₁₋₆alkyl, and C₁₋₆haloalkyl;
Y is C₃₋₈ carbocycle, 5-8 membered heterocycle, —(C₂ aliphatic group)-R¹, C₆₋₁₀ aryl, or 5-10 membered heteroaryl, wherein each of said carbocycle, heterocycle, aryl and heteroaryl is optionally and independently substituted with one or more instances of J^{Y} independently selected from the group consisting of halogen, —CN, nitro, azido, R^{a}, —SO₂R^{a}, —OR^{a}, —COR^{a}, —NRR^{a}, —C(O)OR^{a}, —OC(O)R^{a}, —NRC(O)R^{a}, —C(O)NRR^{a}, —NRC(O)NRR^{a}, —NRC(O)OR^{a}, —OCONRR^{a}, —SO₂NRR^{a}, —NRSO₂R^{a}, —NRSO₂NRR^{a}, and —NRC(═NR)NRR^{a}, and wherein said C₂ aliphatic group is optionally substituted with one or more substituents selected from the group consisting of halogen, —CN, C₁₋₂alkyl, C₁₋₂ haloalkyl, hydroxy, and methoxy;
R¹ is i) —H; ii) a C₁₋₆ alkyl group optionally substituted with one or more instances of J^{1A}; iii) a C₃₋₁₀ carbocycle or 4-10 membered heterocycle, each of which is optionally and independently substituted with one or more instances of J^{1B}; or iv) a C₆₋₁₀ aryl or 5-10 membered heteroaryl group, each of which is optionally and independently substituted with one or more instances of J^{1C};
R² is i) a C₁₋₆ aliphatic group optionally substituted with one or more instances of J^{2A}; ii) a C₃₋₁₀ carbocycle or 4-10 membered heterocycle, each of which is optionally and independently substituted with one or more instances of J^{2B}; or iii) a C₆₋₁₀ aryl or 5-10 membered heteroaryl group, each of which is optionally and independently substituted with one or more instances of J^{2C};
each of R³, R⁴, R⁵ and R⁶ independently is —H, -D, or a C₁₋₆ aliphatic group optionally substituted with one or more instances of J^{D};
each of R⁷ and R⁸ independently is i) —H; ii) a C₁₋₆ aliphatic group optionally substituted with one or more instances of J^{7A}; iii) a C₃₋₁₀ carbocycle or 4-10 membered heterocycle, each of which is optionally and independently substituted with one or more instances of J^{7B}; or iv) a C₆₋₁₀ aryl or 5-10 membered heteroaryl group, each of which is optionally and independently substituted with one or more instances of J^{7C}; or
R⁷ and R⁸, together with the nitrogen atom to which they are attached, form a 4-10 membered heterocyclic ring optionally substituted with one or more instances of J^{E}; or
optionally, when Y is —(C₂ aliphatic group)-R¹, R³ and R⁷, together with the atoms to which they are attached, form a 4-10 membered heterocyclic ring optionally substituted with one or more instances of J^{E}; and
R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C};
each of J^{1A}, J^{2A}, J^{7A}, and J^{9A} independently is oxo or Q; or two J^{1A}, two J^{2A}, two J^{7A}, and two J^{9A}, respectively, together with the atom(s) to which they are attached, optionally and independently form a 3-8-membered non-aromatic ring that is optionally substituted with one or more instances of J^{E};
each of J^{1B}, J^{2B}, J^{7B}, and J^{9B} and independently is oxo, Q, or a C₁₋₆ aliphatic group optionally substituted with one or more instances of Q; or two J^{1B}, two J^{2B}, two J^{3B}, two J^{7B}, and two J^{9B}, respectively, together with the atom(s) to which they are attached, optionally and independently form a 3-8-membered non-aromatic ring that is optionally substituted with one or more instances of J^{E};
each of J^{1C}, J^{2C}, J^{7C} and J^{9C} independently is Q or a C₁₋₆ aliphatic group optionally substituted with one or more instances of Q; or two J^{1C}, two J^{2C}, two J^{7C}, and two J^{9C}, respectively, together with the atom(s) to which they are attached, optionally and independently form a 3-8-membered non-aromatic ring that is optionally substituted with one or more instances of J^{E};
each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F};
each R^{a} independently is: i) —H; ii) a C₁₋₆ aliphatic group optionally substituted with one or more substituents independently selected from the group consisting of halogen, oxo, —CN, —OR, —NRR′, —OCOR, —COR″, —CO₂R, —CONRR′, —NRC(O)R, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or
R^{a}, together with R and the nitrogen atom to which it is attached, optionally forms a 4-8 membered heterocycle optionally substituted with one or more instances of J^{E}; and
each R is independently —H or a C₁₋₆ aliphatic group optionally substituted with one or more instances of J^{D};
each R′ is independently —H or a C₁₋₆ aliphatic group optionally substituted with one or more instances of J^{D}; or R′, together with R and the nitrogen atom to which it is attached, optionally forms a 4-8 membered heterocycle optionally substituted with one or more instances of J^{E};
each R″ is a C₁₋₆ aliphatic group optionally substituted with one or more instances of J^{D};
each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇ cyclo(haloalkyl), and phenyl;
each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D};
each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}; and
n is 0 or 1.

2. (canceled)

3. The compound of claim 1, represented by Structural Formula (II): 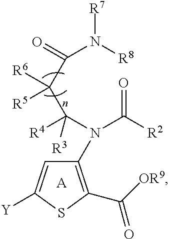
or a pharmaceutically acceptable salt thereof, wherein:
Y is —(C₂ aliphatic group)-R¹ or phenyl optionally substituted one or more of J^{Y}, and wherein said C₂ aliphatic group is optionally substituted;
each J^{Y} is independently selected from the group consisting of halogen, —CN, nitro, R^{a}, —OR^{a}, —COR^{a}, and —NRR^{a};
R¹ is an optionally substituted C₁₋₆ alkyl or C₃₋₈ cycloalkyl, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl;
R² is optionally substituted C₅-C₈ cycloalkyl or optionally substituted phenyl;
each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —OC(O)OR^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl;
R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 5-8 membered heterocyclic ring;
each of J^{1A}, J^{2A}, J^{7A}, and J^{9A} independently is halogen, oxo, —CN, —OR^{a}, —NRR^{a}, —OCOR^{a}, —OC(O)OR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), 5-6 membered optionally substituted heterocyclyl, or optionally substituted phenyl;
each of J^{1B}, J^{2B}, J^{7B}, and J^{9B} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl; and
each of J^{1C}, J^{2C}, J^{7C}, and J^{9C} independently is halogen, oxo, —CN, —OR^{a}, —NRR^{a}, —OC(O)R^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl; and
R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OC(O)(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

4-7. (canceled)

8. The compound of claim 3, wherein:
R¹ is C₁₋₆ alkyl optionally substituted with one or more substituents selected from the group consisting of halogen, —CN, —OH, and —O(C₁-C₆ alkyl);
R² is C₅-C₈ cycloalkyl optionally substituted with one or more substituents selected from the group consisting of halogen; oxo; —CN; —OH; —NH₂; —NH(C₁-C₆ alkyl); —N(C₁-C₆ alkyl)₂: —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl), —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.
each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₇NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl;
each of J^{1A}, J^{2A}, J^{7A}, and J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl;
each of J^{1B}, J^{2B}, J^{7B}, and J^{9B} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl; and
each of J^{1C}, J^{2C}, J^{7C}, and J^{9C} independently is halogen, oxo, —CN, —OR^{a}, —NRR^{a}, —OC(O)R^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

9-17. (canceled)

18. The compound of claim 8, wherein R² is cyclohexyl optionally substituted with one or more instances of J^{2B} independently selected from the group consisting of halogen, —CN, —OH, —NH₂, —NH(C₁₋₆ alkyl), —N(C₁₋₆ alkyl)₂, —O(C₁₋₆ alkyl), and C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆haloalkyl).

19-30. (canceled)

31. The compound of claim 18, wherein Y is —CH₂—CH₂—R¹, —CH═CH—R¹, or —C≡CR¹, or optionally substituted phenyl.

32-39. (canceled)

40. The compound of claim 31, represented by Structural Formula (V): 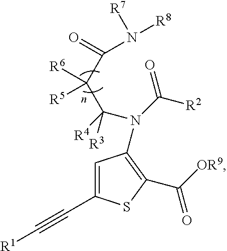
or a pharmaceutically acceptable salt thereof, wherein:
each of R³, R⁴, R⁵ and R⁶ independently is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; and
each of R⁷ and R⁸ independently is —H; C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; or a C₃₋₈ carbocyclic or 4-8 membered heterocyclic group each of which optionally and independently substituted with one or more substituents selected from the group consisting of halogen, oxo, —CN, —OH, —NH, —NH(C₁-C₆ alkyl), —N(C₁-C₆ alkyl), —OCO(C₁-C₆ alkyl), —CO(C₁-C₆ alkyl), —CO₇H, —CO(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂—NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; or
R⁷ and R⁸, together with the atom to which they are attached, optionally form a 4-10 membered heterocyclic ring optionally substituted with one or more substituents selected from the group consisting of halogen, oxo, —CN, —OH, —NH₂, —NH(C₁-C₆ alkyl), —N(C₁-C₆ alkyl)₂-OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; or
R³ and R⁷, together with the atoms to which they are attached, optionally form a 4-10 membered heterocyclic ring optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —C₂(C₁-C₆ alkyl), —O(C₁-C₆ alkyl), —O(C₁-C₆ haloalkyl), and C₁-C₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

41-60. (canceled)

61. The compound of claim 40, wherein:
(a) each of R³, R⁴, R⁵ and R⁶ independently is —H or optionally substituted C₁₋₆alkyl; and
each of R⁷ and R⁸ independently is —H or an optionally substituted C₁₋₆ alkyl, optionally substituted C₃₋₈ carbocyclic, or optionally substituted 4-8 membered heterocyclic group; or R⁷ and R⁸, together with the atom to which they are attached, optionally form an optionally substituted, 4-10 membered heterocyclic ring; or
(b) each of R³, R⁴, R⁵ and R⁶ independently is —H or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl); and
R⁷, and R⁸ independently is —H or C₁₋₆ alkyl optionally substituted with one or more substituents selected from the group consisting of halogen, —CN, —OH, —NH, —NH(C₁₋₆ alkyl), —N(C₁₋₆ alkyl)₂, —O(C₁₋₆ alkyl), and —O(C₁₋₆ haloalkyl); or
R⁷ and R⁸, together with the atom to which they are attached, optionally form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl); or
(c) each of R⁴, R⁵, R⁶, R⁸ independently is —H or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl); and
R³ and R⁷, together with the atoms to which they are attached, form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl).

62. (canceled)

63. The compound of claim 40, wherein R⁷ and R⁸, together with the atom to which they are attached, form an optionally substituted heterocyclic ring, wherein the heterocyclic ring formed with R⁷ and R⁸ is a bridged or spiro ring.

64-66. (canceled)

67. The compound of claim 63, wherein the heterocyclic ring formed with R³ and R⁷ is selected from: 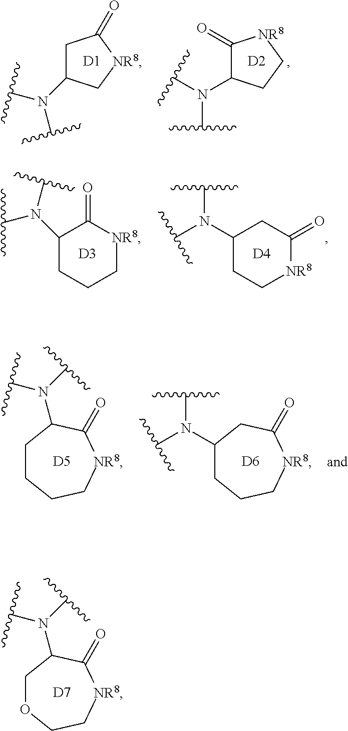
wherein each of rings D1-D7 is independently and optionally further substituted.

68. The compound of claim 40, wherein:
R¹ is C₁₋₆alkyl optionally substituted with one or more substituents selected from the group consisting of halogen, —CN, —OH, and —O(C₁-C₆ alkyl);
R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group;
each of R³, R⁴, R⁵ and R⁶ independently is —H or an optionally substituted C₁₋₆alkyl group; and
each of R⁷ and R⁸ independently is —H, an optionally substituted C₁₋₆ aliphatic, optionally substituted C₃₋₈ carbocyclic; or
optionally R³ and R⁷, together with the atoms to which they are attached, form an optionally substituted, 4-10 membered heterocyclic ring; or
optionally R⁷ and R⁸, together with the nitrogen atom to which they are attached, form an optionally substituted, 4-10 membered heterocyclic ring.

69. The compound of claim 61, represented by Structural Formula (VI): 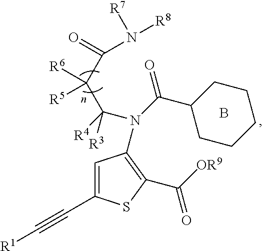
or a pharmaceutically acceptable salt thereof, wherein:
ring B is optionally substituted with one or more instances of J^{2B};
R⁹ is —H or C₁₋₆ alkyl optionally substituted with —OC(O)(C₁-C₆ alkyl);
each of R³, R⁴, R⁵ and R⁶ independently is —H or C₁₋₆ alkyl optionally substituted with one or more substituents selected from the group consisting of halogen, —CN, —OH, —NH₂, —NH(C₁₋₆ alkyl), —N(C₁₋₆ alkyl), —O(C₁₋₆ alkyl), and —O(C₁₋₆ haloalkyl);
J^{2B} is C₁₋₆ alkyl or —O(C₁₋₆ alkyl);
R¹ is C₁₋₆ alkyl.

70-73. (canceled)

74. The compound of claim 73, wherein R⁹ is H or C₁₋₆ alkyl; and R¹ is t-butyl or isopropyl.

75-79. (canceled)

80. The compound of claim 74, wherein each of R³, R⁴, R⁵ and R⁶ independently is —H, —CH₃, —CH₂CH₃, —CH₂CH₂CH₃, —CH₂—OCH₃, —CH₂CH₂—OCH₃, —CH₂CH₂—OCH₂CH₃, or —CH₂CH₂—OCH₂CH₃.

81-82. (canceled)

83. The compound of claim 3, wherein the compound is represented by Structural Formula (X): 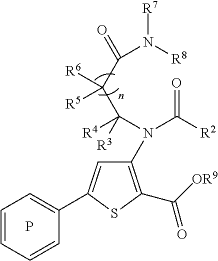
or a pharmaceutically acceptable salt thereof, wherein Ring P is independently and optionally further substituted, wherein:
R² is optionally substituted C₅-C₈ cycloalkyl or optionally substituted phenyl, each of which optionally and independently is substituted with one or more substituents selected from the group consisting of halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H: —CO₂(C₆-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl;
each of R³, R⁴, R⁵ and R⁶ independently is —H or C₁₋₆ alkyl optionally substituted with one or more substituents selected from the group consisting of halogen, —CN, —OH, —NH₇, —NH(C₁₋₆ alkyl), —N(C₁₋₆ alkyl), —O(C₁₋₆ alkyl), and —O(C₁₋₆haloalkyl); and
R⁷, and R⁸ independently is —H or C₁₋₆ alkyl optionally substituted with one or more substituents selected from the group consisting of halogen, —CN, —OH, —NH, —NH(C₁— alkyl), —N(C₁₋₆ alkyl)₂, —O(C₁₋₆ alkyl), and —O(C₁₋₆ haloalkyl); or
R⁷ and R⁸, together with the atom to which they are attached, optionally form a 4-10 membered heterocyclic ring optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl).

84-89. (canceled)

90. The compound of claim 83, represented by Structural Formula (XIII): 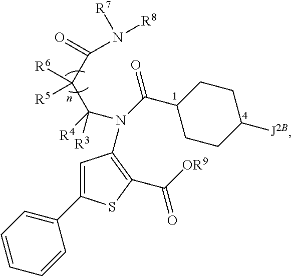
or a pharmaceutically acceptable salt thereof, wherein:
R⁹ is —H or C₁₋₆ alkyl optionally substituted with —OC(O)(C₁-C₆ alkyl);
each of R³, R⁴, R⁵ and R⁶ independently is —H or C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl);
J^{2B} is C₁₋₆ alkyl.

91-95. (canceled)

96. The compound of claim 90, wherein each of R³, R⁴, R⁵ and R⁶ independently is —H, —CH₃, —CH₂CH₃, —CH₂CH₂CH₃, —CH₂—OCH₃, —CH₂CH₂—OCH₃, —CH₂CH₂—OCH₂CH₃, or —CH₂CH₂—OCH₂CH₃.

97-103. (canceled)

104. The compound of claim 1, wherein n is 0.

105. The compound of claim 1, represented by Structural Formula (XX) 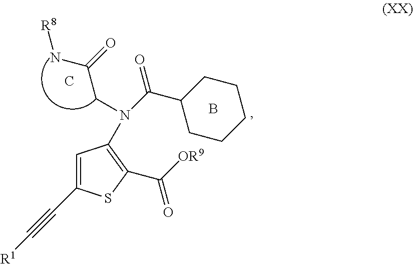
or a pharmaceutically acceptable salt thereof, wherein:
R¹ is C₁₋₆alkyl optionally substituted with one or more substituents selected from the group consisting of halogen, —CN, —OH, and —O(C₁-C₆ alkyl);
R⁸ is —H or C₁₋₄ alkyl optionally substituted with one or substituents selected from the group consisting of halogen, hydroxyl, —O(C₁₋₄alkyl), —NH₂, —NH(C₁₋₄ alkyl), and —N(C₁₋₄ alkyl)₂;
R⁹ is —H or C₁₋₆alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —OC(O)O(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇cycloalkyl, C₃₋₇cyclo(haloalkyl), phenyl, and 5-6-membered heterocycle optionally substituted with one or more substituents selected from the group consisting of oxo and C₁-C₆ alkyl;
ring B is optionally substituted with one or more instances of J^{2B}; and
ring C is a 5-7 membered heterocycle optionally substituted with one or substituents selected from the group consisting of halogen, hydroxyl, —O(C₁₋₄alkyl), —NH2, —NH(C₁₋₄ alkyl), and —N(C₁₋₄ alkyl)₂.

106. (canceled)

107. The compound of claim 106, wherein:
R¹ is t-butyl or isopropyl;
R⁸ is —H or C₁₋₄ alkyl optionally substituted with one or substituents selected from the group consisting of halogen, hydroxyl, —O(CH₃), —O(C₂H₅), —NH₂, —NH(CH₃), and —N(CH₃)₂;
R⁹ is —H or C₁₋₆ alkyl optionally substituted with one or more substituents selected from the group consisting of halogen, oxo, —CN, —OH, —NH, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; and
J^{2B} is halogen, —CN, —OH, —NH₂, —NH(C₁₋₆ alkyl), —N(C₁₋₆ alkyl)₂, —O(C₁₋₆alkyl), or C₁₋₆alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆alkyl), and —O(C₁₋₆haloalkyl).

108. The compound of claim 107, wherein R⁸ is —H or C₁₋₄alkyl.

109. The compound of claim 108, wherein R⁹ is —H.

110. The compound of claim 109, wherein J^{2B} is —CH₃ or —O(CH₃).

111. The compound of claim 110, wherein the compound is represented by Structural formula (XXI): 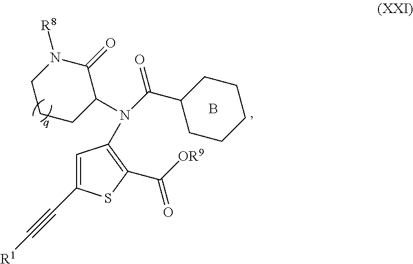
or a pharmaceutically acceptable salt thereof, wherein q is 0, 1, or 2.

112. (canceled)

113. A compound selected from any one of the structural formulae depicted in FIG. 1, FIG. 2 or FIG. 3, or a pharmaceutically acceptable salt thereof.

114. (canceled)

115. (canceled)

116. A pharmaceutical composition, comprising a compound of claim 1 and a pharmaceutically acceptable carrier or excipient.

117. A method of inhibiting or reducing the activity of HCV polymerase in a biological in vitro sample, comprising administering to the sample an effective amount of a compound of claim 1.

118. A method of treating a HCV infection in a subject, comprising administering to the subject a therapeutically effective amount of a compound of claim 1.

119. A method of inhibiting or reducing the activity of HCV polymerase in a subject, comprising administering to the subject a therapeutically effective amount of a compound of claim 1.

120-131. (canceled)

132. A method of preparing a compound represented by Structural Formula (I) or a pharmaceutically acceptable salt thereof, wherein the variables of Structural Formula (I) are each and independently as described in claim 1, and wherein the method comprises the step of reacting compound (1g) with compound (1h) to form a compound of Structural Formula (I): 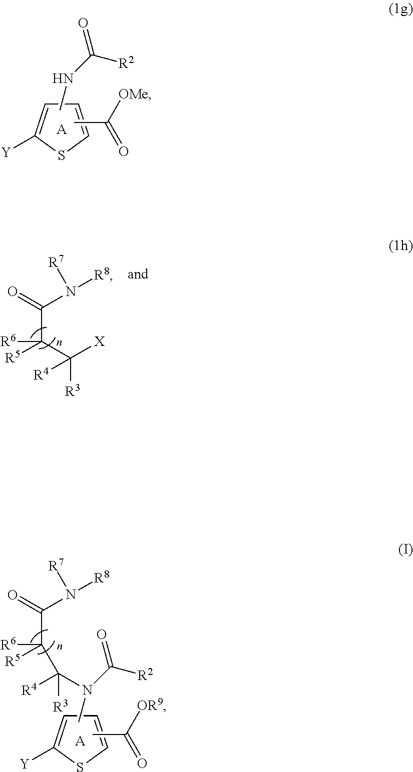
wherein the variables of compounds (1g) and (1h) are each and independently as described for Structural Formula (I), and X is a suitable leaving group; and
optionally further comprises the step of hydrolyzing the —C(O)OR⁹ of the compound of Structural Formula (I) under a suitable hydrolysis condition to form —COOH.

133. (canceled)

134. (canceled)

135. The method of claim 132, wherein the compound prepared by the method is represented by Structural Formula (IV) or a pharmaceutically acceptable salt thereof: 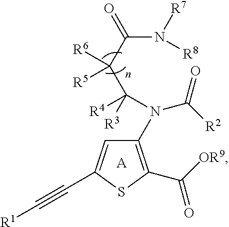
wherein the variables of Structural Formula (IV) are each and independently as described in claim 132 for Structural Formula (I).

136. A method of preparing a compound represented by Structural Formula (I) or a pharmaceutically acceptable salt thereof, wherein the variables of Structural Formula (I) are each and independently as described in claim 1, and wherein the method comprises the step of reacting compound (1p) with compound (1f): X—C(O)R² to form a compound of Structural Formula (I): 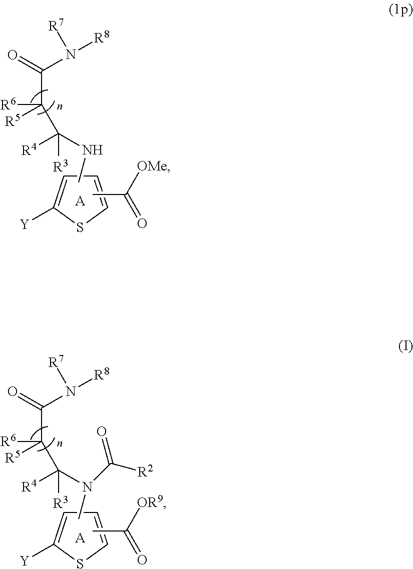
wherein the variables of compound (1p) are each and independently as described for Structural Formula (I), and X is a suitable leaving group; and
optionally further comprises the step of hydrolyzing the —C(O)OR⁹ of the compound of Structural Formula (I) under a suitable hydrolysis condition to form —COOH.

137. (canceled)

138. (canceled)

139. The method of claim 136, wherein the compound prepared by the method is represented by Structural Formula (IV) or a pharmaceutically acceptable salt thereof: 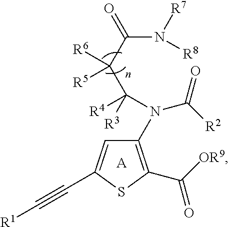
wherein the variables of Structural Formula (II) are each and independently as described in claim 136 for Structural Formula (I).
